(19) United States
(12) Patent Application Publication
(10) Pub. No.: US 2024/0389455 A1
(43) Pub. Date: Nov. 21, 2024

(54) HETEROCYCLIC COMPOUND, ORGANIC LIGHT-EMITTING DEVICE COMPRISING SAME, AND COMPOSITION FOR ORGANIC MATERIAL LAYER OF ORGANIC LIGHT-EMITTING DEVICE

(71) Applicant: LT MATERIALS CO., LTD., Yongin-si, Gyeonggi-do (KR)

(72) Inventors: Young-Beom SEO, Yongin-si (KR); Geon-Yu PARK, Yongin-si (KR); Young-Seok NO, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/289,357

(22) PCT Filed: Jan. 18, 2022
(86) PCT No.: PCT/KR2022/000885

§ 371 Date: Nov. 2, 2023

(30) Foreign Application Priority Data
May 11, 2021 (KR) 10-2021-0060919

(51) Int. Cl. H10K 85/60; C07D 405/14; C07D 487/04; C07D 491/048; C07D 495/04; C09K 11/06; H10K 50/12

(52) U.S. Cl. CPC H10K 85/6572; C07D 405/14; C07D 487/04; C07D 491/048; C07D 495/04; C09K 11/06; H10K 85/615; H10K 85/626; H10K 85/654; H10K 85/657; H10K 85/6574; C09K 2211/1018; H10K 50/12

(57) ABSTRACT

The present application provides a heterocyclic compound, an organic light emitting device in which the heterocyclic compound is contained in an organic material layer and a composition for an organic material layer of the organic light emitting device.

### TECHNICAL FIELD

This application claims priority to and the benefit of Korean Patent Application No. 10-2021-0060919 filed in the Korean Intellectual Property Office on May 11, 2021, the entire contents of which are incorporated herein by reference.

The present specification relates to a heterocyclic compound, an organic light emitting device comprising the same and a composition for an organic material layer of the organic light emitting device.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multi layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENT

### Patent Document

U.S. Pat. No. 4,356,429

### DISCLOSURE

### Technical Problem

The present invention has been made in an effort to provide a heterocyclic compound, an organic light emitting device comprising the same and a composition for an organic material layer of the organic light emitting device.

### Technical Solution

An exemplary embodiment of the present application provides a heterocyclic compound represented by the following Formula 1.

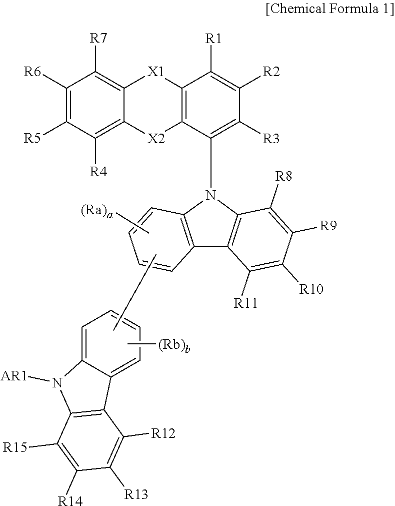

In Chemical Formula 1,
one of X1 and X2 is a direct bond, and the other is O,
   R1 to R15, Ra and Rb are the same as or different from each other, and are each independently hydrogen; deuterium; a substituted or unsubstituted alkyl group having 1 to 60 carbon atoms; a substituted or unsubstituted aryl group having 6 to 60 carbon atoms; a substituted or unsubstituted heteroaryl group having 2 to 60 carbon atoms; or —NRcRd, and Rc and Rd are the same as or different from each other, and are each independently a substituted or unsubstituted aryl group having 6 to 60 carbon atoms; or a substituted or unsubstituted heteroaryl group having 2 to 60 carbon atoms,
   at least one of R1 to R3 is a substituted or unsubstituted aryl group having 6 to 60 carbon atoms, AR1 is a substituted or unsubstituted aryl group having 6 to 60 carbon atoms,
   when X2 is O, R1 is hydrogen; or deuterium, and
   a and b are an integer from 0 to 3, and when a is 2 or higher, Ra's in the parenthesis are the same as or different from each other, and when b is 2 or higher, Rb's in the parenthesis are the same as or different from each other.

Further, another exemplary embodiment of the present application provides an organic light emitting device comprising a first electrode, a second electrode and an organic material layer having one or more layers provided between the first electrode and the second electrode, in which one or more layers of the organic material layer comprise the heterocyclic compound represented by Chemical Formula 1.

In addition, still another exemplary embodiment of the present application provides a composition for an organic material layer of the organic light emitting device comprising the heterocyclic compound represented by Chemical Formula 1.

### Advantageous Effects

A heterocyclic compound according to an exemplary embodiment of the present application can be used as a material for an organic material layer of an organic light emitting device. The heterocyclic compound can be used as a material for a hole injection layer, a hole transport layer, a light emitting layer, an electron transport layer, an electron injection layer, a charge generation layer, and the like in an organic light emitting device. In particular, the heterocyclic compound represented by Chemical Formula 1 can be used as a material for the light emitting layer of the organic light emitting device. In addition, when the heterocyclic compound represented by Chemical Formula 1 is used for an organic light emitting device, the driving voltage of the device can be lowered, the light efficiency of the device can be improved, and the service life characteristics of the device can be improved due to the thermal stability of the compound.

### DESCRIPTION OF DRAWINGS

FIGS. 1 to 3 each are views schematically illustrating a stacking structure of an organic light emitting device according to an exemplary embodiment of the present application.

### MODE FOR INVENTION

Hereinafter, the present application will be described in detail.

An exemplary embodiment of the present application provides a heterocyclic compound represented by the following Formula 1.

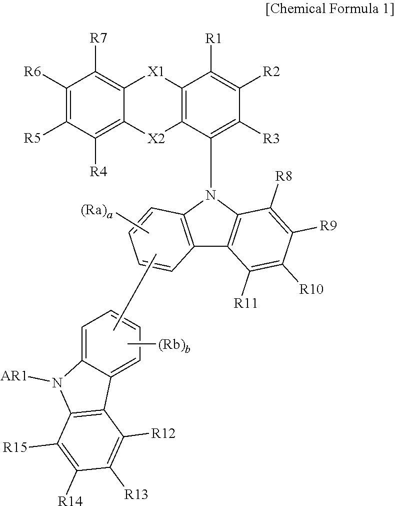

In Chemical Formula 1,
one of X1 and X2 is a direct bond, and the other is O,
   R1 to R15, Ra and Rb are the same as or different from each other, and are each independently hydrogen; deuterium; a substituted or unsubstituted alkyl group having 1 to 60 carbon atoms; a substituted or unsubstituted aryl group having 6 to 60 carbon atoms; a substituted or unsubstituted heteroaryl group having 2 to 60 carbon atoms; or —NRcRd, and Rc and Rd are the same as or different from each other, and are each independently a substituted or unsubstituted aryl group having 6 to 60 carbon atoms; or a substituted or unsubstituted heteroaryl group having 2 to 60 carbon atoms,
   at least one of R1 to R3 is a substituted or unsubstituted aryl group having 6 to 60 carbon atoms, AR1 is a substituted or unsubstituted aryl group having 6 to 60 carbon atoms,
   when X2 is O, R1 is hydrogen; or deuterium, and
   a and b are an integer from 0 to 3, and when a is 2 or higher, Ra's in the parenthesis are the same as or different from each other, and when b is 2 or higher, Rb's in the parenthesis are the same as or different from each other.

The heterocyclic compound represented by Chemical Formula 1 has a bulkier structure by introducing an additional heteroaryl group into a basic biscarbazole structure and allowing at least one of R1 to R3 of the introduced heteroaryl group to have an aryl substituent. Therefore, the region of a highest occupied molecular orbital (HOMO) level is expanded more widely, and when the HOMO level is increased, there is an effect that the overall charge balance in a device is stabilized by adjusting the hole mobility.

Further, when the HOMO level is increased, there is an advantage in that it is possible to enhance the stability of the molecular structure and to be utilized as an auxiliary means which adjusts the energy band gap because at least one of R1 to R3 has a wide n-conjugation region in an expanded form by having an aryl group.

In addition, since a heteroaryl group such as dibenzofuran has a more rigid structure while having a wide n-conjugation region in a form similar to an aryl group, it is possible to see an effect capable of maintaining a high T1 energy level and simultaneously adjusting the energy band gap. Furthermore, the heteroaryl group has a high structural stability, and thus helps to have longer service life characteristics. T1 means a triplet.

Due to these characteristics, when the heterocyclic compound represented by Chemical Formula 1 is used as a material for an organic material layer of an organic light emitting device, there is an advantage in that the driving voltage of the device is lowered, the optical efficiency is improved, and the service life characteristics of the device may be improved.

In the present specification, the term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more substituents are substituted, the two or more substituents may be the same as or different from each other.

In the present specification, “substituted or unsubstituted” means being unsubstituted or substituted with one or more substituents selected from the group consisting of deuterium; a halogen group; a cyano group; a straight-chained or branched alkyl having 1 to 60 carbon atoms; a straight-chained or branched alkenyl having 2 to 60 carbon atoms; a straight-chained or branched alkynyl having 2 to 60 carbon atoms; a monocyclic or polycyclic cycloalkyl having 3 to 60 carbon atoms; a monocyclic or polycyclic heterocycloalkyl having 2 to 60 carbon atoms; a monocyclic or polycyclic aryl having 6 to 60 carbon atoms; a monocyclic or polycyclic heteroaryl having 2 to 60 carbon atoms; —SiRR′R″; —P(═O)RR′; an alkylamine having 1 to 20 carbon atoms; a monocyclic or polycyclic arylamine having 6 to 60 carbon atoms; and a monocyclic or polycyclic heteroarylamine having 2 to 60 carbon atoms, or being unsubstituted or substituted with a substituent to which two or more substituents selected from the above exemplified substituents are linked, and means that R, R′ and R″ are the same as or different from each other, and are each independently hydrogen; deuterium; a halogen; a substituted or unsubstituted alkyl having 1 to 60 carbon atoms; a substituted or unsubstituted aryl having 6 to 60 carbon atoms; or a substituted or unsubstituted heteroaryl having 2 to 60 carbon atoms.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen; or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or 2H.

In an exemplary embodiment of the present application, the isotope mean an atom with the same atomic number (Z), but different mass numbers (A), and the isotope may be interpreted as an element which has the same number of protons, but different number of neutrons.

In an exemplary embodiment of the present application, when the total number of substituents of a basic compound is defined as T1 and the number of specific substituents among the substituents is defined as T2, the meaning of the content T % of the specific substituent may be defined as T2/T1×100=T %.

That is, in an example, the deuterium content of 20% in a phenyl group represented by

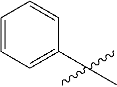 [SMILES: *c1ccccc1]

means that the total number of substituents that the phenyl group can have is 5 (T1 in the formula), and may be represented by 20% when the number of deuteriums among the substituents is 1 (T2 in the formula). That is, a deuterium content of 20% in the phenyl group may be represented by the following structural formula.

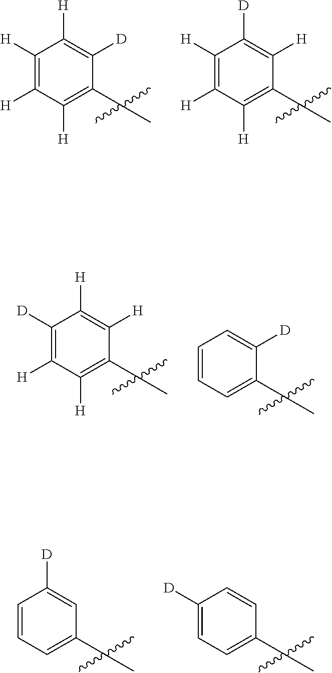 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not comprise a deuterium atom, that is, has five hydrogen atoms.

In the present specification, the halogen may be fluorine, chlorine, bromine or iodine.

In the present specification, the alkyl group comprises a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof comprise a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, the alkenyl group comprises a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof comprise a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl) vinyl-1-yl group, a 2,2-bis(diphenyl-1-yl) vinyl-1-yl group, a stilbenyl group, a styrenyl group, and the like, but are not limited thereto.

In the present specification, the alkynyl group comprises a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, an alkoxy group may be straight-chained, branched, or cyclic. The number of carbon atoms of the alkoxy group is not particularly limited, but is preferably 1 to 20. Specific examples thereof comprise methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, the cycloalkyl group comprises a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof comprise a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group comprises O, S, Se, N, or Si as a heteroatom, comprises a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, the aryl group comprises a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group comprises a spiro group. The number of carbon atoms of the aryl group may be 6 to 60, specifically 6 to 40, and more specifically 6 to 25. Specific examples of the aryl group comprise a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, a silyl group comprises Si and is a substituent to which the Si atom is directly linked as a radical, and is represented by —SiR101R102R103, and R101 to R103 are the same as or different from each other, and may be each independently a substituent composed of at least one of hydrogen; deuterium; a halogen group; an alkyl group; an alkenyl group; an alkoxy group; a cycloalkyl group; an aryl group; and a heterocyclic group. Specific examples of the silyl group comprise a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a phosphine oxide group is represented by —P(═O)R104R105, and R104 and R105 are the same as or different from each other, and may be each independently a substituent composed of at least one of hydrogen; deuterium; a halogen group; an alkyl group; an alkenyl group; an alkoxy group; a cycloalkyl group; an aryl group; and a heterocyclic group. Specific examples of the phosphine oxide group comprise a diphenylphosphine oxide group, dinaphthylphosphine oxide, and the like, but are not limited thereto.

In the present specification, the fluorenyl group may be substituted, and adjacent substituents may be bonded to each other to form a ring.

In the present specification, the spiro group is a group comprising a spiro structure, and may have 15 to 60 carbon atoms. For example, the spiro group may comprise a structure in which a 2,3-dihydro-1H-indene group or a cyclohexane group is spiro-bonded to a fluorenyl group. Specifically, the spiro group may comprise any one of the groups of the following structural formulae.

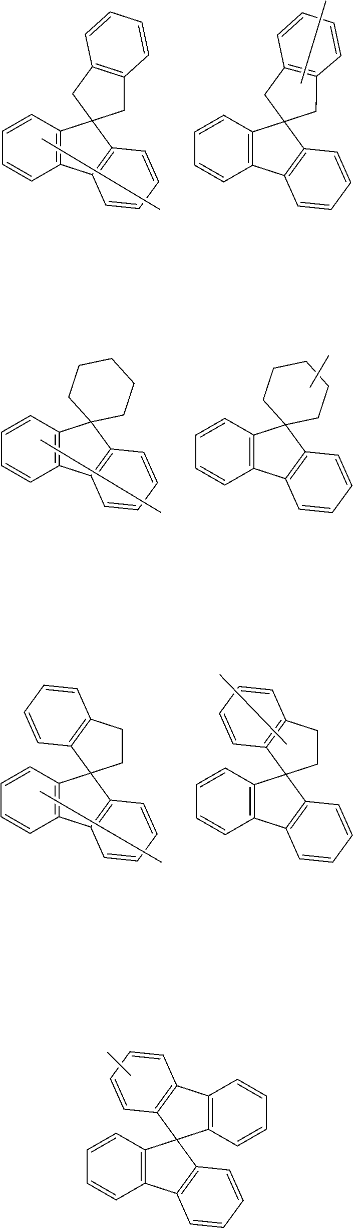

In the present specification, the heteroaryl group comprises S, O, Se, N, or Si as a heteroatom, comprises a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group comprise a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a] carbazolyl group, an indolo[2,3-b] carbazolyl group, an indolinyl group, a 10,11-dihydro-dibenzo[b, f] azepin group, a 9,10-dihydroacridinyl group, a phenanthrazinyl group, a phenothiazinyl group, a phthalazinyl group, a naphthylidinyl group, a phenanthrolinyl group, a benzo[c] [1,2,5] thiadiazolyl group, a 5,10-dihydrodibenzo[b, e] [1, 4] azasilinyl, a pyrazolo[1,5-c] quinazolinyl group, a pyrido[1,2-b] indazolyl group, a pyrido[1,2-a] imidazo[1,2-e] indolinyl group, a 5,11-dihydroindeno[1,2-b] carbazolyl group, and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group comprise a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, an arylene group means that there are two bonding positions in an aryl group, that is, a divalent group. The above-described description on the aryl group may be applied to the arylene group, except that the arylene groups are each a divalent group. Further, a heteroarylene group means that there are two bonding positions in a heteroaryl group, that is, a divalent group. The above-described description on the heteroaryl group may be applied to the heteroarylene group, except for a divalent heteroarylene group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted with the same carbon in an aliphatic ring may be interpreted as groups which are “adjacent” to each other.

A heterocyclic compound according to an exemplary embodiment of the present application is represented by Chemical Formula 1. More specifically, the heterocyclic compound represented by Chemical Formula 1 may be used as a material for an organic material layer of an organic light emitting device by the structural characteristics of the core structure and the substituent as described above.

In an exemplary embodiment of the present application, one of X1 and X2 of Chemical Formula 1 is a direct bond, and the other is O.

In an exemplary embodiment of the present application, X1 is a direct bond, and X2 is O.

In an exemplary embodiment of the present application, X2 is a direct bond, and X1 is O.

In an exemplary embodiment of the present application, R1 to R15, Ra and Rb of Chemical Formula 1 are the same as or different from each other, and are each independently hydrogen; deuterium; a substituted or unsubstituted alkyl group having 1 to 60 carbon atoms; a substituted or unsubstituted aryl group having 6 to 60 carbon atoms; a substituted or unsubstituted heteroaryl group having 2 to 60 carbon atoms; or —NRcRd, and Rc and Rd are the same as or different from each other, and are each independently a substituted or unsubstituted aryl group having 6 to 60 carbon atoms; or a substituted or unsubstituted heteroaryl group having 2 to 60 carbon atoms, and at least one of R1 to R3 may be a substituted or unsubstituted aryl group having 6 to 60 carbon atoms.

In an exemplary embodiment of the present application, R1 to R15, Ra and Rb are the same as or different from each other, and are each independently hydrogen; deuterium; a substituted or unsubstituted alkyl group having 1 to 40 carbon atoms; a substituted or unsubstituted aryl group having 6 to 40 carbon atoms; a substituted or unsubstituted heteroaryl group having 2 to 40 carbon atoms; or —NRCRd, and Rc and Rd are the same as or different from each other, and are each independently a substituted or unsubstituted aryl group having 6 to 40 carbon atoms; or a substituted or unsubstituted heteroaryl group having 2 to 40 carbon atoms, and at least one of R1 to R3 may be a substituted or unsubstituted aryl group having 6 to 40 carbon atoms.

In an exemplary embodiment of the present application, R1 to R15, Ra and Rb are the same as or different from each other, and are each independently hydrogen; deuterium; a substituted or unsubstituted alkyl group having 1 to 20 carbon atoms; a substituted or unsubstituted aryl group having 6 to 20 carbon atoms; a substituted or unsubstituted heteroaryl group having 2 to 20 carbon atoms; or —NRcRd, and Rc and Rd are the same as or different from each other, and are each independently a substituted or unsubstituted aryl group having 6 to 20 carbon atoms; or a substituted or unsubstituted heteroaryl group having 2 to 20 carbon atoms, and at least one of R1 to R3 may be a substituted or unsubstituted aryl group having 6 to 20 carbon atoms.

In an exemplary embodiment of the present application, R1 to R15, Ra and Rb are the same as or different from each other, and are each independently hydrogen; deuterium; or a substituted or unsubstituted aryl group having 6 to 60 carbon atoms, and at least one of R1 to R3 may be a substituted or unsubstituted aryl group having 6 to 60 carbon atoms.

In an exemplary embodiment of the present application, R1 to R15, Ra and Rb are the same as or different from each other, and are each independently hydrogen; deuterium; or a substituted or unsubstituted aryl group having 6 to 40 carbon atoms, and at least one of R1 to R3 may be a substituted or unsubstituted aryl group having 6 to 40 carbon atoms.

In an exemplary embodiment of the present application, R1 to R15, Ra and Rb are the same as or different from each other, and are each independently hydrogen; deuterium; or a substituted or unsubstituted aryl group having 6 to 20 carbon atoms, and at least one of R1 to R3 may be a substituted or unsubstituted aryl group having 6 to 20 carbon atoms.

In an exemplary embodiment of the present application, when R1 to R15, Ra, Rb and AR1 have a substituent substituted with deuterium, the contents of substituted deuterium of R1 to R15, Ra, Rb and AR1 may be each independently 0% to 100%.

In an exemplary embodiment of the present application, when R1 to R15, Ra, Rb and AR1 have a substituent substituted with deuterium, the content of substituted deuterium of R1 to R15, Ra, Rb and AR1 may be each independently 0% or 100%.

In an exemplary embodiment of the present application, R1 to R3 are the same as or different from each other, and are each independently hydrogen; deuterium; or a substituted or unsubstituted aryl group having 6 to 60 carbon atoms, R4 to R15, Ra and Rb are the same as or different from each other, and are each independently hydrogen; or deuterium, and at least one of R1 to R3 may be a substituted or unsubstituted aryl group having 6 to 60 carbon atoms.

In an exemplary embodiment of the present application, R1 to R3 are the same as or different from each other, and are each independently hydrogen; deuterium; or a substituted or unsubstituted aryl group having 6 to 40 carbon atoms, R4 to R15, Ra and Rb are the same as or different from each other, and are each independently hydrogen; or deuterium, and at least one of R1 to R3 may be a substituted or unsubstituted aryl group having 6 to 40 carbon atoms.

In an exemplary embodiment of the present application, R1 to R3 are the same as or different from each other, and are each independently hydrogen; deuterium; or a substituted or unsubstituted aryl group having 6 to 20 carbon atoms, R4 to R15, Ra and Rb are the same as or different from each other, and are each independently hydrogen; or deuterium, and at least one of R1 to R3 may be a substituted or unsubstituted aryl group having 6 to 20 carbon atoms.

In an exemplary embodiment of the present application, R1 to R3 are the same as or different from each other, and are each independently hydrogen; deuterium; a substituted or unsubstituted phenyl group; a substituted or unsubstituted biphenyl group; or a substituted or unsubstituted naphthyl group, R4 to R15, Ra and Rb are the same as or different from each other, and are each independently hydrogen; or deuterium, and at least one of R1 to R3 may be a substituted or unsubstituted phenyl group; a substituted or unsubstituted biphenyl group; or a substituted or unsubstituted naphthyl group.

In an exemplary embodiment of the present application, R1 to R3 are the same as or different from each other, and are each independently hydrogen; deuterium; a phenyl group which is unsubstituted or substituted with one or more deuteriums; a biphenyl group which is unsubstituted or substituted with one or more deuteriums; or a naphthyl group which is unsubstituted or substituted with one or more deuteriums, R4 to R15, Ra and Rb are the same as or different from each other, and are each independently hydrogen; or deuterium, and at least one of R1 to R3 may be a phenyl group which is unsubstituted or substituted with one or more deuteriums; a biphenyl group which is unsubstituted or substituted with one or more deuteriums; or a naphthyl group which is unsubstituted or substituted with one or more deuteriums.

In an exemplary embodiment of the present application, when R1 to R3 are a phenyl group which is unsubstituted or substituted with one or more deuteriums; a biphenyl group which is unsubstituted or substituted with one or more deuteriums; or a naphthyl group which is unsubstituted or substituted with one or more deuteriums, the contents of substituted deuterium of R1 to R3 may be each independently 0% to 100%.

In an exemplary embodiment of the present application, when R1 to R3 are a phenyl group which is unsubstituted or substituted with one or more deuteriums; a biphenyl group which is unsubstituted or substituted with one or more deuteriums; or a naphthyl group which is unsubstituted or substituted with one or more deuteriums, the content of substituted deuterium of R1 to R3 may be each independently 0% or 100%.

In an exemplary embodiment of the present application, R4 to R15, Ra and Rb are the same as or different from each other, and may be each independently hydrogen; deuterium; or a substituted or unsubstituted aryl group having 6 to 40 carbon atoms.

In an exemplary embodiment of the present application, R4 to R15, Ra and Rb are the same as or different from each other, and may be each independently hydrogen; deuterium; or a substituted or unsubstituted aryl group having 6 to 20 carbon atoms.

In an exemplary embodiment of the present application, R4 to R15, Ra and Rb are the same as or different from each other, and are each independently hydrogen; or deuterium.

In an exemplary embodiment of the present application, all of R4 to R15, Ra and Rb are hydrogen.

In an exemplary embodiment of the present application, all of R4 to R15, Ra and Rb are deuterium.

In an exemplary embodiment of the present application, when X1 of Chemical Formula 1 is O, R2 and R3 are hydrogen; or deuterium, and R1 may be a substituted or unsubstituted aryl group having 6 to 60 carbon atoms.

In an exemplary embodiment of the present application, when X1 is O, R1 and R3 are hydrogen; or deuterium, and R2 may be a substituted or unsubstituted aryl group having 6 to 60 carbon atoms.

In an exemplary embodiment of the present application, when X1 is O, R1 and R2 are hydrogen; or deuterium, and R3 may be a substituted or unsubstituted aryl group having 6 to 60 carbon atoms.

In an exemplary embodiment of the present application, when X2 of Chemical Formula 1 is O, R1 is hydrogen; or deuterium, and at least one of R2 and R3 may be a substituted or unsubstituted aryl group having 6 to 60 carbon atoms.

When R1 of the compound represented by Chemical Formula 1 of the present application is hydrogen; or deuterium, the structural stability of the compound becomes excellent, so that a device in which the compound is used has an effect in which the service life of the device is better.

In an exemplary embodiment of the present application, when X2 of Chemical Formula 1 is O, R1 and R2 are hydrogen; or deuterium, and R3 may be a substituted or unsubstituted aryl group having 6 to 60 carbon atoms.

In an exemplary embodiment of the present application, when X2 is O, R1 and R3 are hydrogen; or deuterium, and R2 may be a substituted or unsubstituted aryl group having 6 to 60 carbon atoms.

In an exemplary embodiment of the present application, when X2 is O, R1 is hydrogen; or deuterium, and at least one of R2 and R3 may be a substituted or unsubstituted aryl group having 6 to 60 carbon atoms.

In an exemplary embodiment of the present application, AR1 may be a substituted or unsubstituted aryl group having 6 to 60 carbon atoms.

In an exemplary embodiment of the present application, AR1 may be a substituted or unsubstituted aryl group having 6 to 40 carbon atoms.

In an exemplary embodiment of the present application, AR1 may be a substituted or unsubstituted aryl group having 6 to 20 carbon atoms.

In an exemplary embodiment of the present application, AR1 may be a substituted or unsubstituted phenyl group; or a substituted or unsubstituted biphenyl group.

In an exemplary embodiment of the present application, AR1 may be a phenyl group which is unsubstituted or substituted with one or more deuteriums; or a biphenyl group which is unsubstituted or substituted with one or more deuteriums.

In an exemplary embodiment of the present application, when AR1 is a phenyl group which is unsubstituted or substituted with one or more deuteriums; or a biphenyl group which is unsubstituted or substituted with one or more deuteriums, the content of substituted deuterium of AR1 may be 0% to 100%.

In an exemplary embodiment of the present application, when AR1 is a phenyl group which is unsubstituted or substituted with one or more deuteriums; or a biphenyl group which is unsubstituted or substituted with one or more deuteriums, the content of substituted deuterium of AR1 may be 0% or 100%.

In an exemplary embodiment of the present application, a and b of Chemical Formula 1 are an integer from 0 to 3, when a is 2 or higher, Ra's in the parenthesis are the same as or different from each other, and when b is 2 or higher, Rb's in the parenthesis are the same as or different from each other.

In an exemplary embodiment of the present application, a of Chemical Formula 1 is an integer from 0 to 3, and when a is 2 or higher, Ra's in the parenthesis may be the same as or different from each other.

In another exemplary embodiment, a is O.

In still another exemplary embodiment, a is 1.

In yet another exemplary embodiment, a is 2.

In yet another exemplary embodiment, a is 3.

In an exemplary embodiment of the present application, when a is 2 or higher, Ra's in the parenthesis may be the same as or different from each other.

In an exemplary embodiment of the present application, b of Chemical Formula 1 is an integer from 0 to 3, and when a is 2 or higher, Rb's in the parenthesis may be the same as or different from each other.

In another exemplary embodiment, b is 0.

In still another exemplary embodiment, b is 1.

In yet another exemplary embodiment, b is 2.

In yet another exemplary embodiment, b is 3.

In an exemplary embodiment of the present application, when b is 2 or higher, Rb's in the parenthesis may be the same as or different from each other.

In an exemplary embodiment of the present application, Chemical Formula 1 may be represented by the following Chemical Formula 1-1 or 1-2.

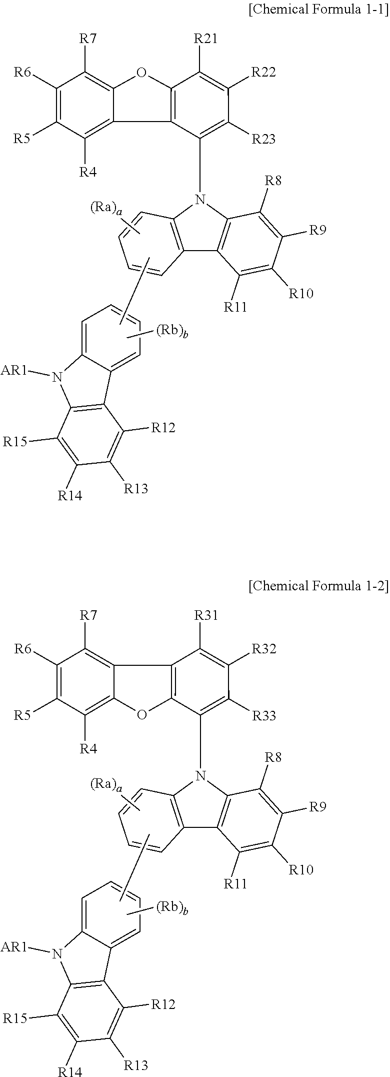

In Formulae 1-1 and 1-2,
R31 is hydrogen; or deuterium,
R21 to R23, R32 and R33 are the same as or different from each other, and are each independently hydrogen; deuterium; a substituted or unsubstituted alkyl group having 1 to 60 carbon atoms; a substituted or unsubstituted aryl group having 6 to 60 carbon atoms; a substituted or unsubstituted heteroaryl group having 2 to 60 carbon atoms; or —NRcRd, and Rc and Rd are the same as or different from each other, and are each independently a substituted or unsubstituted aryl group having 6 to 60 carbon atoms; or a substituted or unsubstituted heteroaryl group having 2 to 60 carbon atoms,
at least one of R21 to R23 is a substituted or unsubstituted aryl group having 6 to 60 carbon atoms,
at least one of R32 and R33 is a substituted or unsubstituted aryl group having 6 to 60 carbon atoms, and
the definitions of R4 to R15, Ra, Rb, AR1, a and b are the same as those in Chemical Formula 1.

In an exemplary embodiment of the present application, R21 to R23 of Chemical Formula 1-1 are the same as or different from each other, and are each independently hydrogen; deuterium; or a substituted or unsubstituted aryl group having 6 to 60 carbon atoms, and at least one of R21 to R23 may be a substituted or unsubstituted aryl group having 6 to 60 carbon atoms.

In an exemplary embodiment of the present application, R21 to R23 are the same as or different from each other, and are each independently hydrogen; deuterium; or a substituted or unsubstituted aryl group having 6 to 40 carbon atoms, and at least one of R21 to R23 may be a substituted or unsubstituted aryl group having 6 to 40 carbon atoms.

In an exemplary embodiment of the present application, R21 to R23 are the same as or different from each other, and are each independently hydrogen; deuterium; or a substituted or unsubstituted aryl group having 6 to 20 carbon atoms, and at least one of R21 to R23 may be a substituted or unsubstituted aryl group having 6 to 20 carbon atoms.

In an exemplary embodiment of the present application, R21 to R23 are the same as or different from each other, and are each independently hydrogen; deuterium; a substituted or unsubstituted phenyl group; a substituted or unsubstituted biphenyl group; or a substituted or unsubstituted naphthyl group, and at least one of R21 to R23 may be a substituted or unsubstituted phenyl group; a substituted or unsubstituted biphenyl group; or a substituted or unsubstituted naphthyl group.

In an exemplary embodiment of the present application, R21 to R23 are the same as or different from each other, and are each independently hydrogen; deuterium; a phenyl group which is unsubstituted or substituted with one or more deuteriums; a biphenyl group which is unsubstituted or substituted with one or more deuteriums; or a naphthyl group which is unsubstituted or substituted with one or more deuteriums, and at least one of R21 to R23 may be a phenyl group which is unsubstituted or substituted with one or more deuteriums; a biphenyl group which is unsubstituted or substituted with one or more deuteriums; or a naphthyl group which is unsubstituted or substituted with one or more deuteriums.

In an exemplary embodiment of the present application, R22 and R23 of Chemical Formula 1-1 are hydrogen; or deuterium, and R21 may be a substituted or unsubstituted aryl group having 6 to 60 carbon atoms.

In an exemplary embodiment of the present application, R21 and R23 of Chemical Formula 1-1 are hydrogen; or deuterium, and R22 may be a substituted or unsubstituted aryl group having 6 to 60 carbon atoms.

In an exemplary embodiment of the present application, R21 and R22 of Chemical Formula 1-1 are hydrogen; or deuterium, and R23 may be a substituted or unsubstituted aryl group having 6 to 60 carbon atoms.

In an exemplary embodiment of the present application, R31 of Chemical Formula 1-2 is hydrogen; or deuterium. In an exemplary embodiment of the present application, R31 is hydrogen.

In an exemplary embodiment of the present application, R31 is deuterium.

In an exemplary embodiment of the present application, R32 and R33 of Chemical Formula 1-2 are the same as or different from each other, and are each independently hydrogen; deuterium; or a substituted or unsubstituted aryl group having 6 to 60 carbon atoms, and at least one of R32 and R33 may be a substituted or unsubstituted aryl group having 6 to 60 carbon atoms.

In an exemplary embodiment of the present application, R32 and R33 are the same as or different from each other, and are each independently hydrogen; deuterium; or a substituted or unsubstituted aryl group having 6 to 40 carbon atoms, and at least one of R32 and R33 may be a substituted or unsubstituted aryl group having 6 to 40 carbon atoms.

In an exemplary embodiment of the present application, R32 and R33 are the same as or different from each other, and are each independently hydrogen; deuterium; or a substituted or unsubstituted aryl group having 6 to 20 carbon atoms, and at least one of R32 and R33 may be a substituted or unsubstituted aryl group having 6 to 20 carbon atoms.

In an exemplary embodiment of the present application, R32 and R33 are the same as or different from each other, and are each independently hydrogen; deuterium; a substituted or unsubstituted phenyl group; a substituted or unsubstituted biphenyl group; or a substituted or unsubstituted naphthyl group, and at least one of R32 and R33 may be a substituted or unsubstituted phenyl group; a substituted or unsubstituted biphenyl group; or a substituted or unsubstituted naphthyl group.

In an exemplary embodiment of the present application, R32 and R33 are the same as or different from each other, and are each independently hydrogen; deuterium; a phenyl group which is unsubstituted or substituted with one or more deuteriums; a biphenyl group which is unsubstituted or substituted with one or more deuteriums; or a naphthyl group which is unsubstituted or substituted with one or more deuteriums, and at least one of R32 and R33 may be a phenyl group which is unsubstituted or substituted with one or more deuteriums; a biphenyl group which is unsubstituted or substituted with one or more deuteriums; or a naphthyl group which is unsubstituted or substituted with one or more deuteriums.

In an exemplary embodiment of the present application, R32 is hydrogen; or deuterium, and R33 may be a substituted or unsubstituted aryl group having 6 to 60 carbon atoms.

In an exemplary embodiment of the present application, R33 is hydrogen; or deuterium, and R32 may be a substituted or unsubstituted aryl group having 6 to 60 carbon atoms.

In an exemplary embodiment of the present application, Chemical Formula 1-1 may be represented by any one of the following Chemical Formulae 1-1-1 to Jan. 1, 2016.

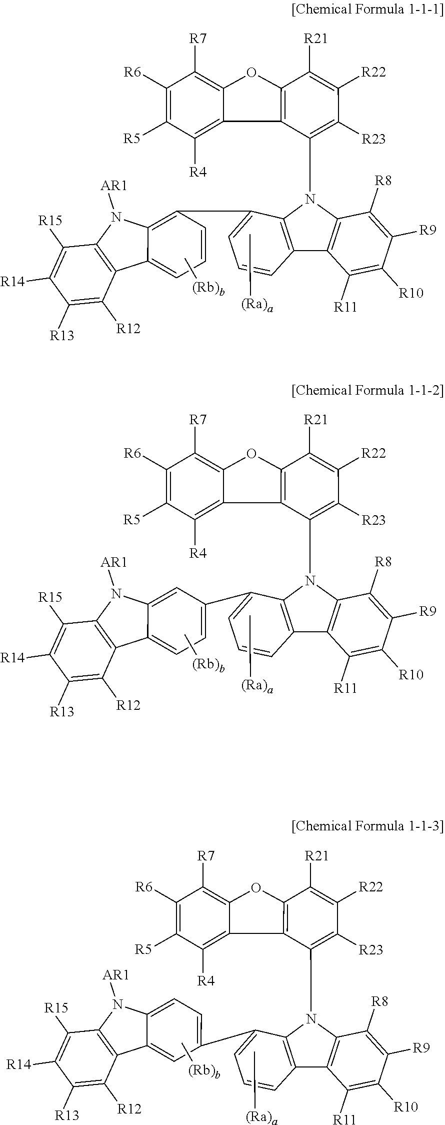

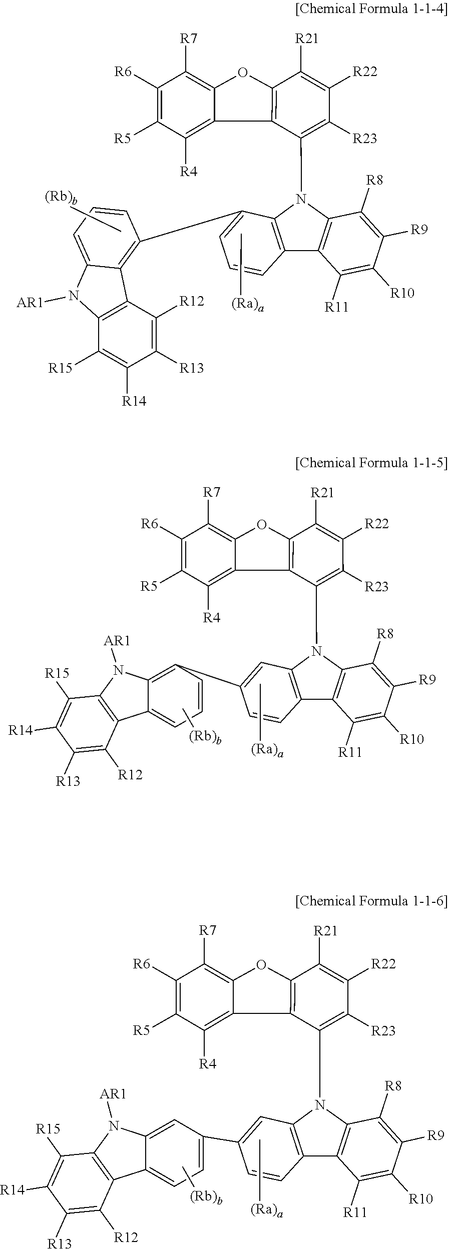

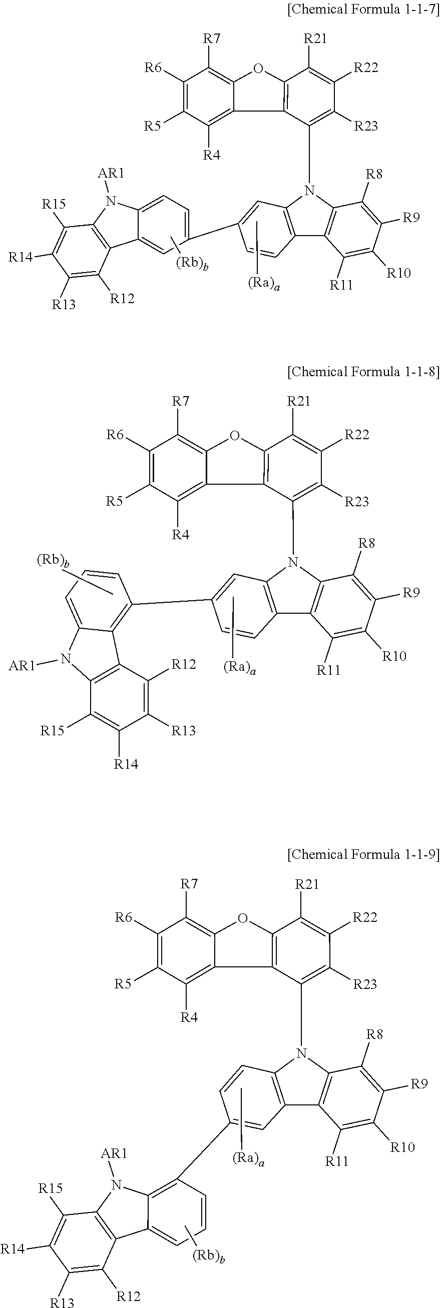

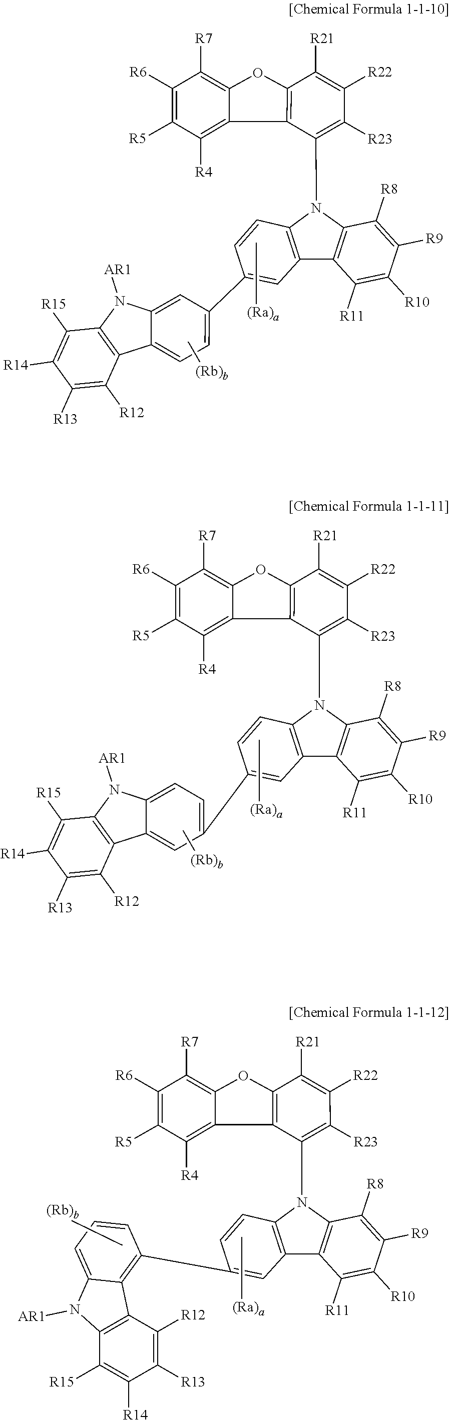

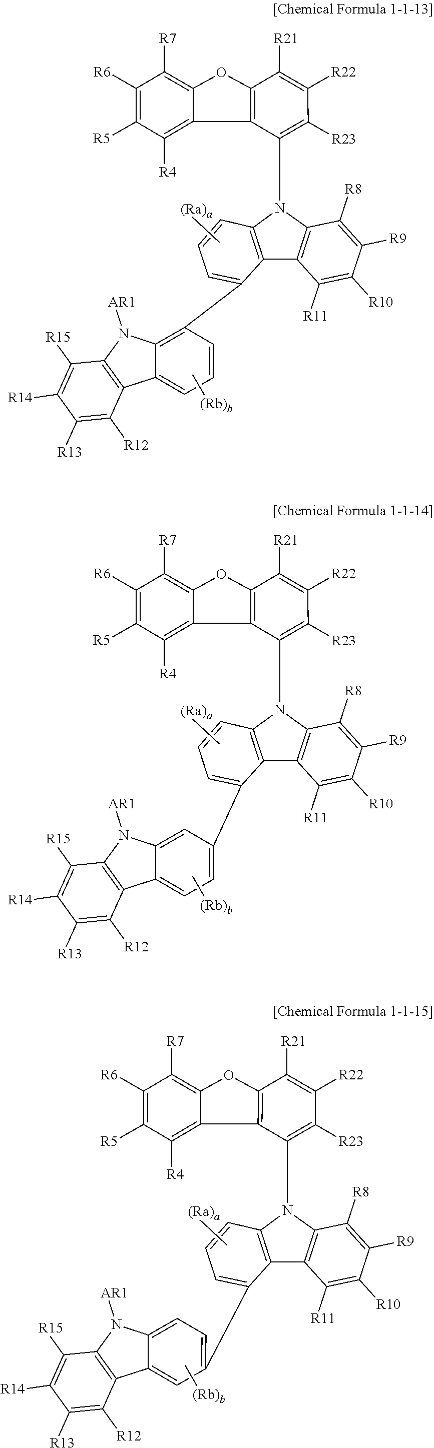

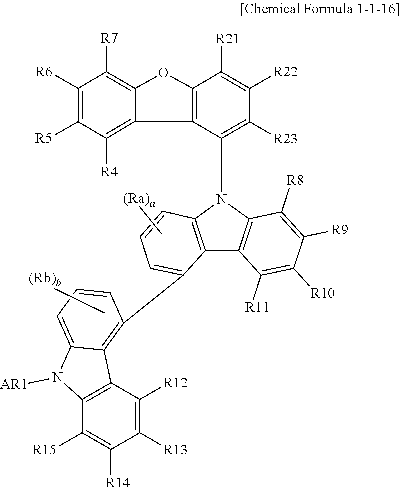

In Chemical Formulae 1-1-1 to Jan. 1, 2016, the definitions of R4 to R15, Ra, Rb, AR1, a, and b and the definitions of R21 to R23 are the same as those in Chemical Formula 1-1.

When the heterocyclic compounds represented by Chemical Formulae 1-1-1 to Jan. 1, 2016 are used as a material for an organic material layer of an organic light emitting device, there is an advantage in that the driving voltage of the device is lowered, the optical efficiency is improved, and the service life characteristics of the device may be improved. Among them, particularly, in the case of a 3,3-biscarbazole bond such as the heterocyclic compound represented by Chemical Formula Jan. 1, 2011, the device may have a better efficiency and a service life due to the appropriate distribution of HOMO and LUMO.

In an exemplary embodiment of the present application, Chemical Formula 1-2 may be represented by any one of the following Chemical Formulae 1-2-1 to Jan. 2, 2016.

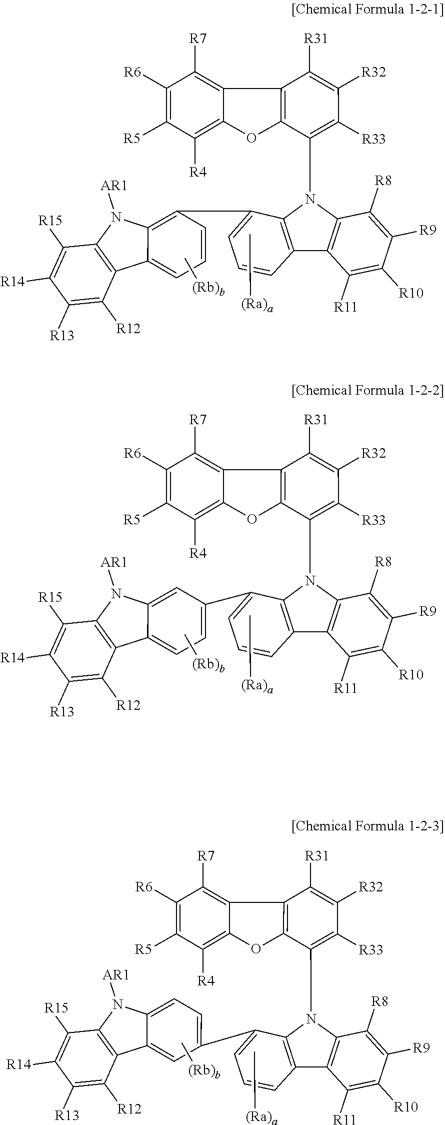

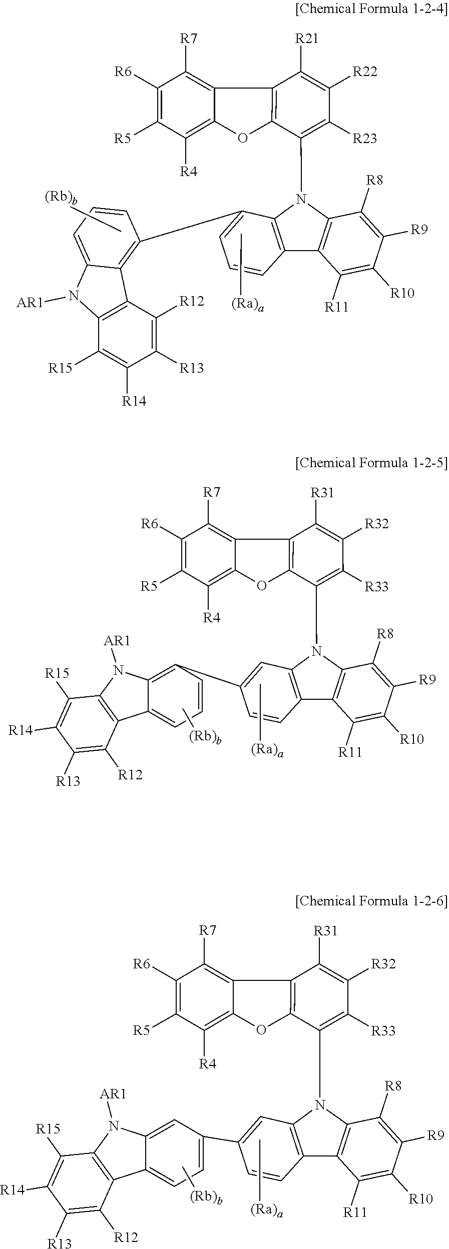

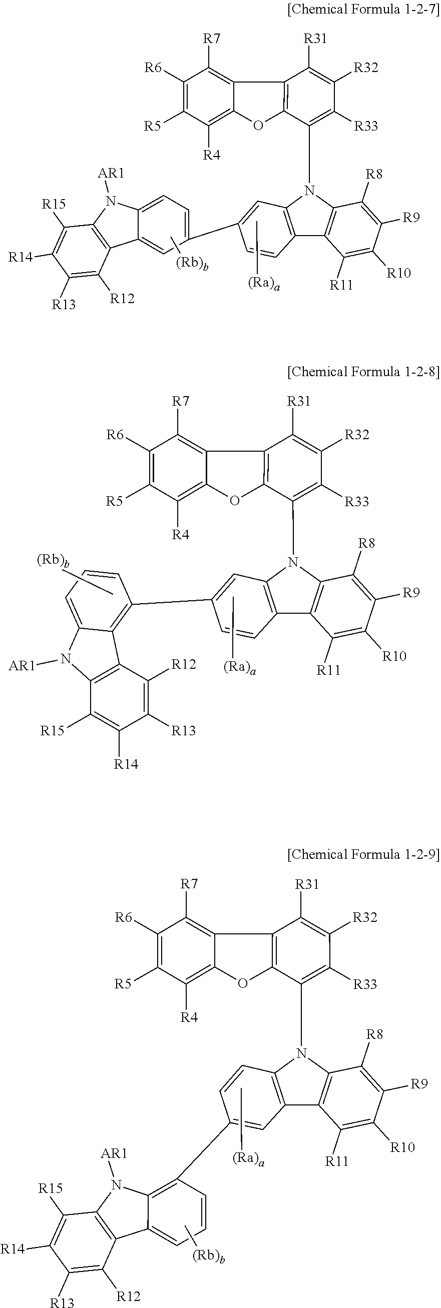

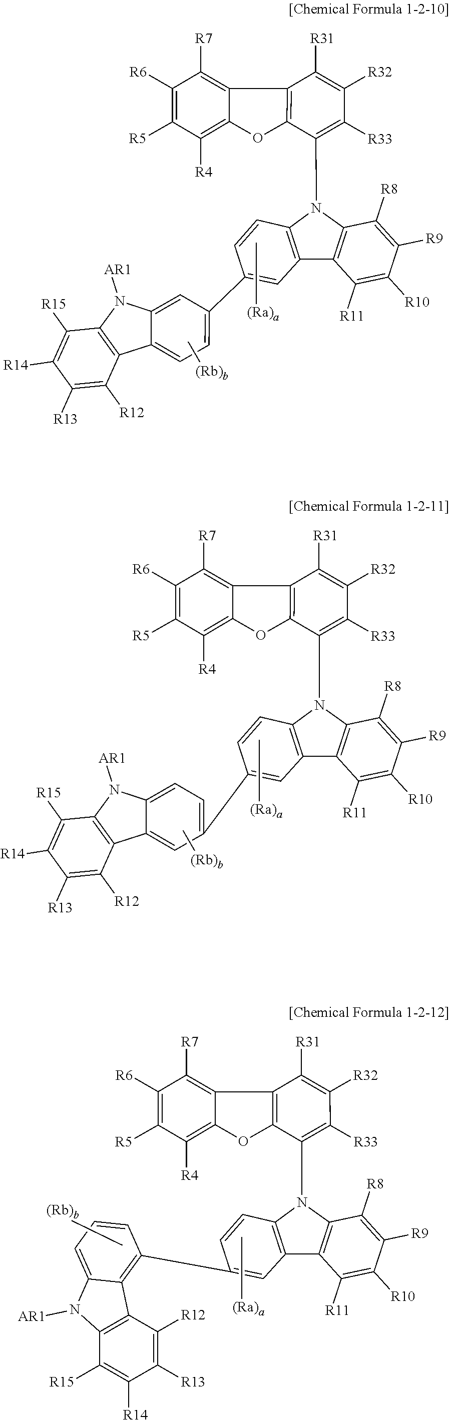

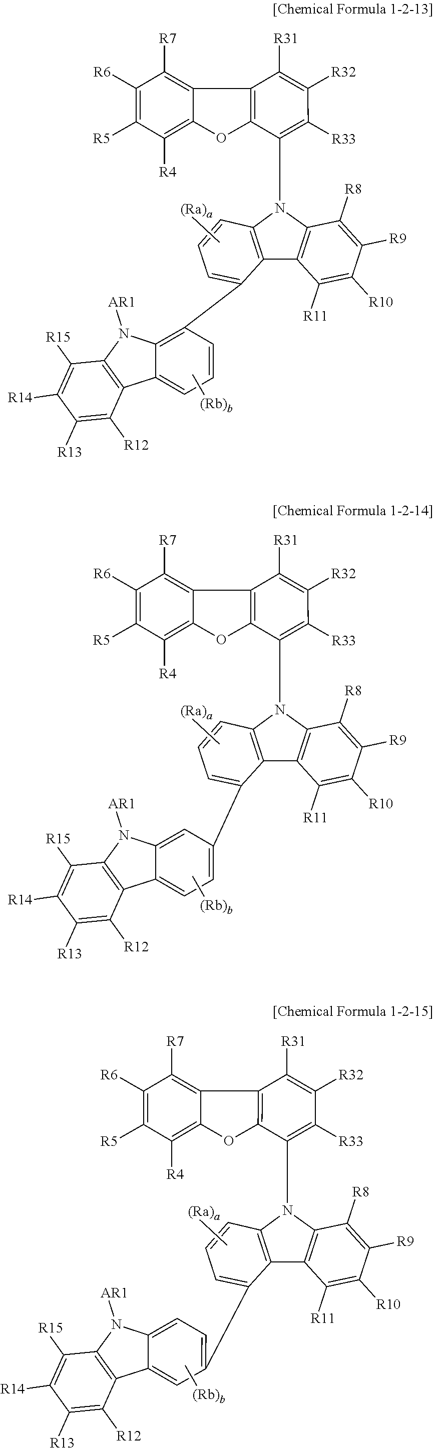

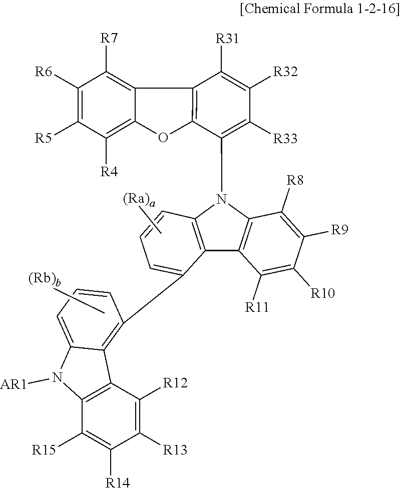

In Chemical Formulae 1-2-1 to Jan. 2, 2016, the definitions of R4 to R15, Ra, Rb, AR1, a, b and R31 to R33 are the same as those in Chemical Formula 1-2.

When the heterocyclic compounds represented by Chemical Formula 1-2-1 to Jan. 2, 2016 are used as a material for an organic material layer of an organic light emitting device, there is an advantage in that the driving voltage of the device is lowered, the optical efficiency is improved, and the service life characteristics of the device may be improved. Among them, particularly, in the case of a 3,3-biscarbazole bond such as the heterocyclic compound represented by Chemical Formula Jan. 2, 2011, the device may have a better efficiency and a service life due to the appropriate distribution of HOMO and LUMO.

According to an exemplary embodiment of the present application, Chemical Formula 1 may be represented by any one of the following compounds, but is not limited thereto.

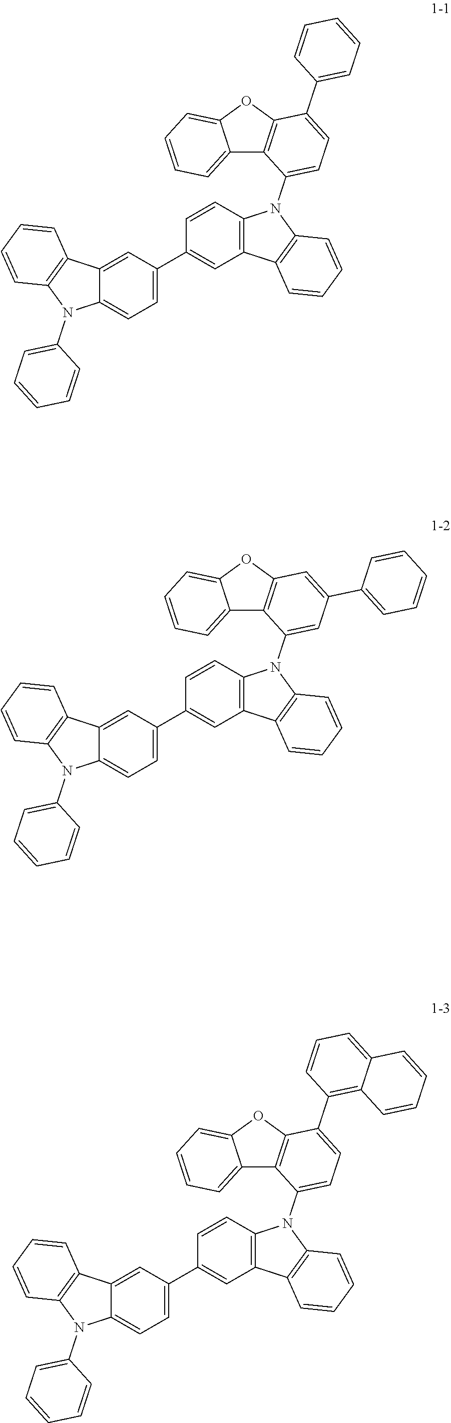 [SMILES: c1ccc(-c2ccc(-n3c4ccccc4c4cc(-c5ccc6c(c5)c5ccccc5n6-c5ccccc5)ccc43)c3c2oc2ccccc23)cc1 ; c1ccc(-c2cc(-n3c4ccccc4c4cc(-c5ccc6c(c5)c5ccccc5n6-c5ccccc5)ccc43)c3c(c2)oc2ccccc23)cc1 ; c1ccc(-n2c3ccccc3c3cc(-c4ccc5c(c4)c4ccccc4n5-c4ccc(-c5cccc6ccccc56)c5oc6ccccc6c45)ccc32)cc1]

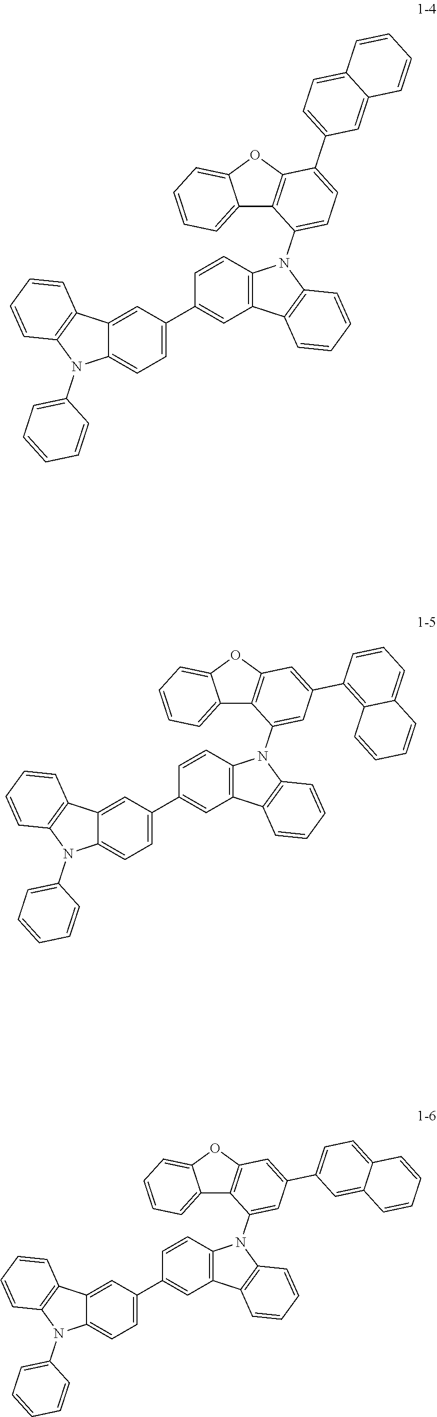 [SMILES: c1ccc(-n2c3ccccc3c3cc(-c4ccc5c(c4)c4ccccc4n5-c4ccc(-c5ccc6ccccc6c5)c5oc6ccccc6c45)ccc32)cc1 ; c1ccc(-n2c3ccccc3c3cc(-c4ccc5c(c4)c4ccccc4n5-c4cc(-c5cccc6ccccc56)cc5oc6ccccc6c45)ccc32)cc1 ; c1ccc(-n2c3ccccc3c3cc(-c4ccc5c(c4)c4ccccc4n5-c4cc(-c5ccc6ccccc6c5)cc5oc6ccccc6c45)ccc32)cc1]

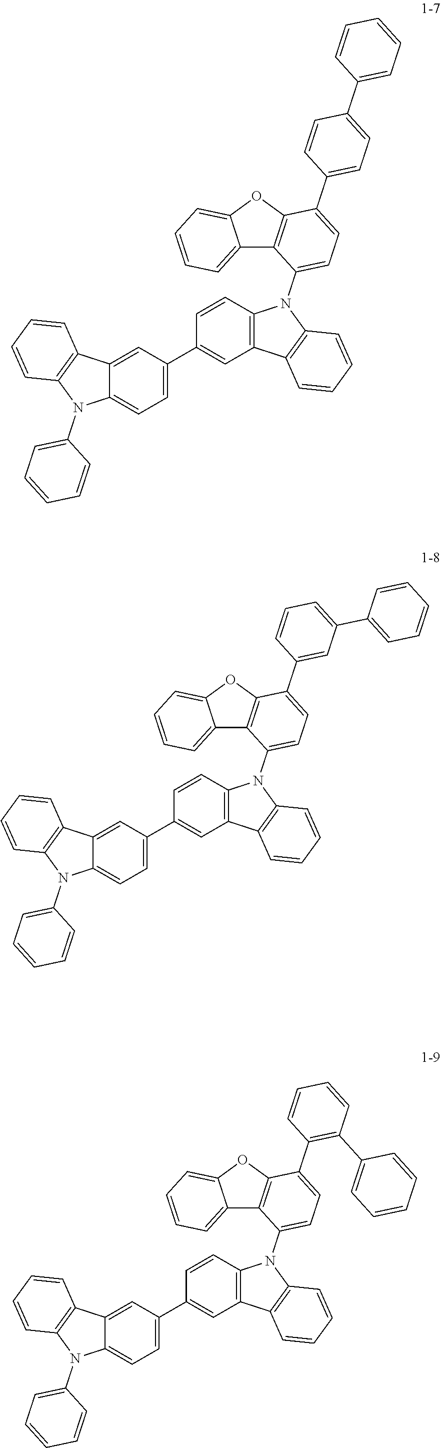 [SMILES: c1ccc(-c2ccc(-c3ccc(-n4c5ccccc5c5cc(-c6ccc7c(c6)c6ccccc6n7-c6ccccc6)ccc54)c4c3oc3ccccc34)cc2)cc1 ; c1ccc(-c2cccc(-c3ccc(-n4c5ccccc5c5cc(-c6ccc7c(c6)c6ccccc6n7-c6ccccc6)ccc54)c4c3oc3ccccc34)c2)cc1 ; c1ccc(-c2ccccc2-c2ccc(-n3c4ccccc4c4cc(-c5ccc6c(c5)c5ccccc5n6-c5ccccc5)ccc43)c3c2oc2ccccc23)cc1]

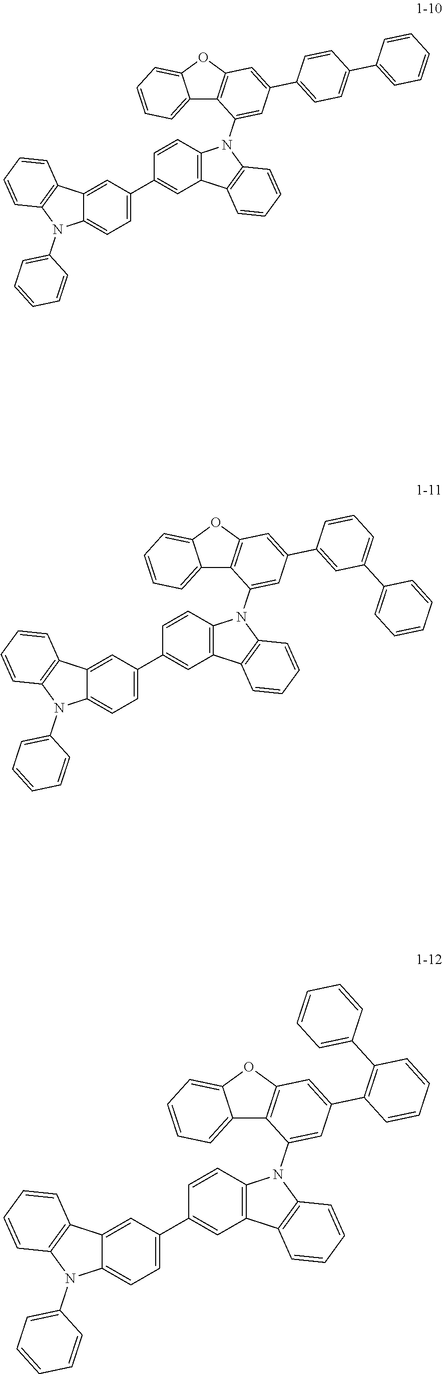 [SMILES: c1ccc(-c2ccc(-c3cc(-n4c5ccccc5c5cc(-c6ccc7c(c6)c6ccccc6n7-c6ccccc6)ccc54)c4c(c3)oc3ccccc34)cc2)cc1 ; c1ccc(-c2cccc(-c3cc(-n4c5ccccc5c5cc(-c6ccc7c(c6)c6ccccc6n7-c6ccccc6)ccc54)c4c(c3)oc3ccccc34)c2)cc1 ; c1ccc(-c2ccccc2-c2cc(-n3c4ccccc4c4cc(-c5ccc6c(c5)c5ccccc5n6-c5ccccc5)ccc43)c3c(c2)oc2ccccc23)cc1]

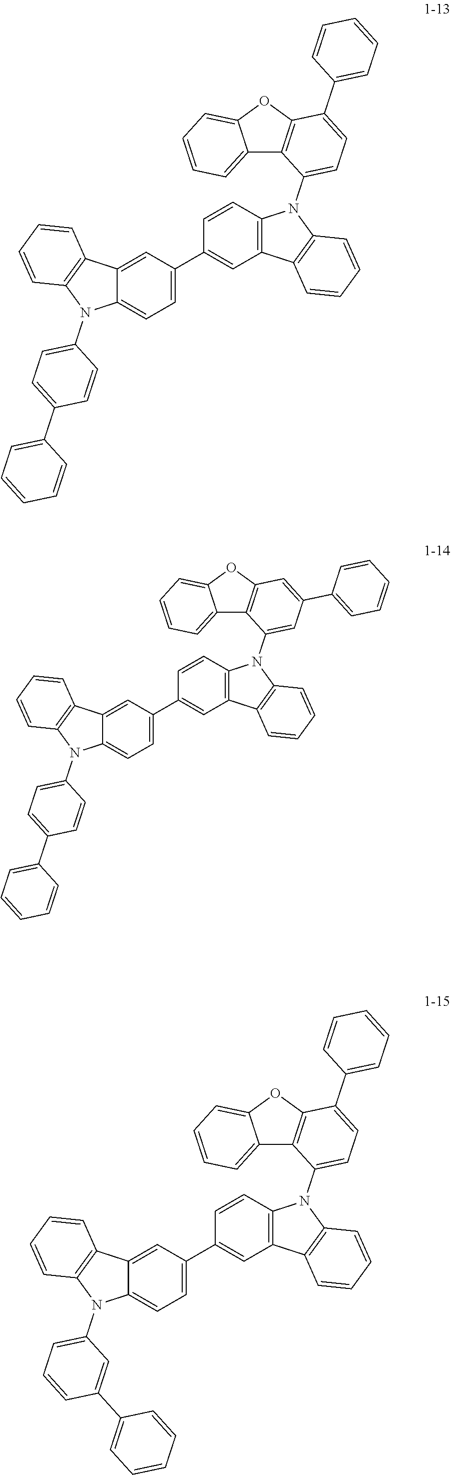 [SMILES: c1ccc(-c2ccc(-n3c4ccccc4c4cc(-c5ccc6c(c5)c5ccccc5n6-c5ccc(-c6ccccc6)c6oc7ccccc7c56)ccc43)cc2)cc1 ; c1ccc(-c2ccc(-n3c4ccccc4c4cc(-c5ccc6c(c5)c5ccccc5n6-c5cc(-c6ccccc6)cc6oc7ccccc7c56)ccc43)cc2)cc1 ; c1ccc(-c2cccc(-n3c4ccccc4c4cc(-c5ccc6c(c5)c5ccccc5n6-c5ccc(-c6ccccc6)c6oc7ccccc7c56)ccc43)c2)cc1]

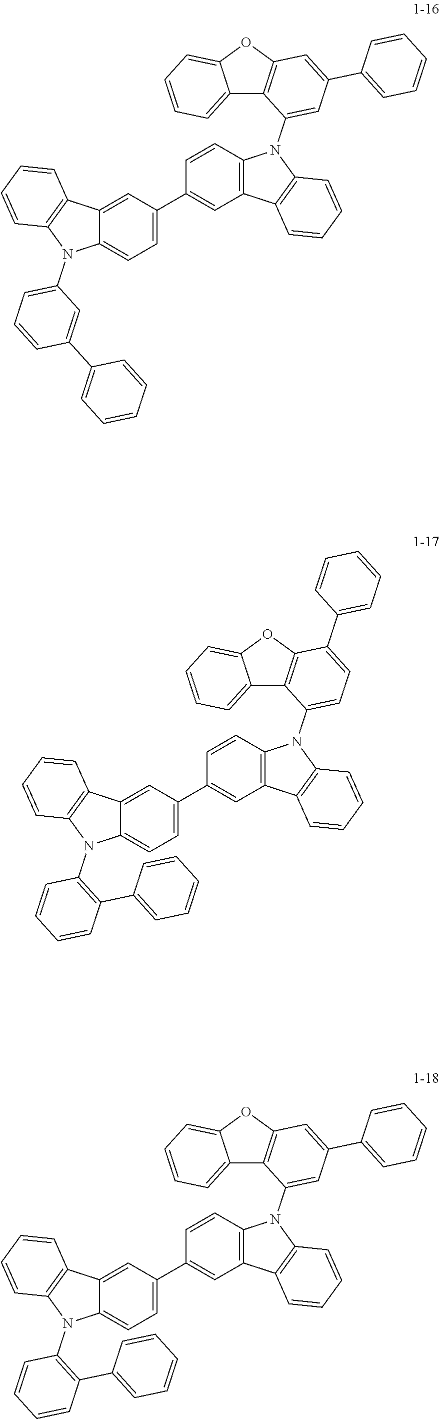 [SMILES: c1ccc(-c2cccc(-n3c4ccccc4c4cc(-c5ccc6c(c5)c5ccccc5n6-c5cc(-c6ccccc6)cc6oc7ccccc7c56)ccc43)c2)cc1 ; c1ccc(-c2ccccc2-n2c3ccccc3c3cc(-c4ccc5c(c4)c4ccccc4n5-c4ccc(-c5ccccc5)c5oc6ccccc6c45)ccc32)cc1 ; c1ccc(-c2cc(-n3c4ccccc4c4cc(-c5ccc6c(c5)c5ccccc5n6-c5ccccc5-c5ccccc5)ccc43)c3c(c2)oc2ccccc23)cc1]

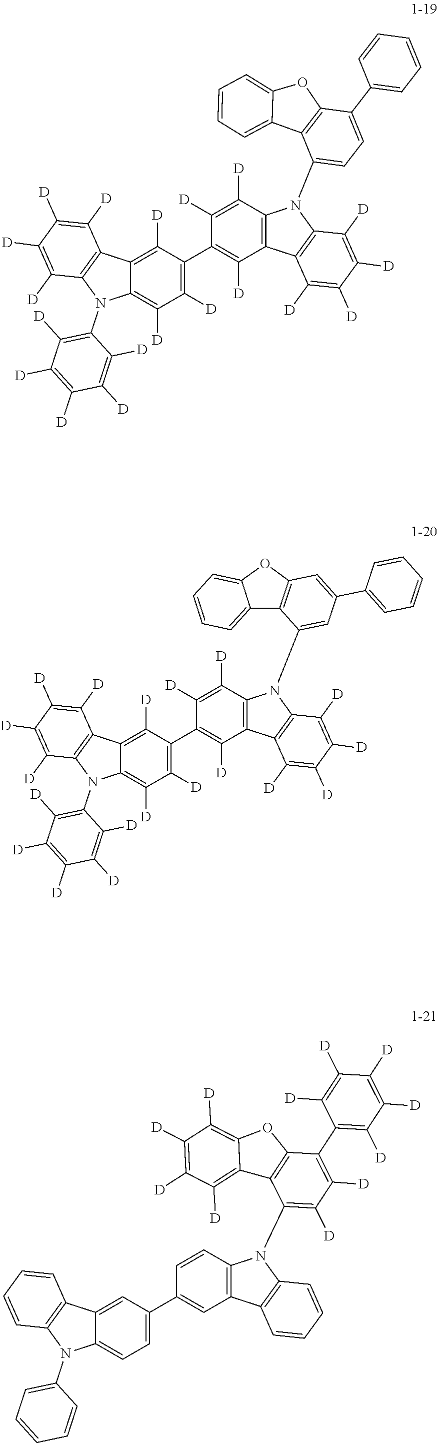

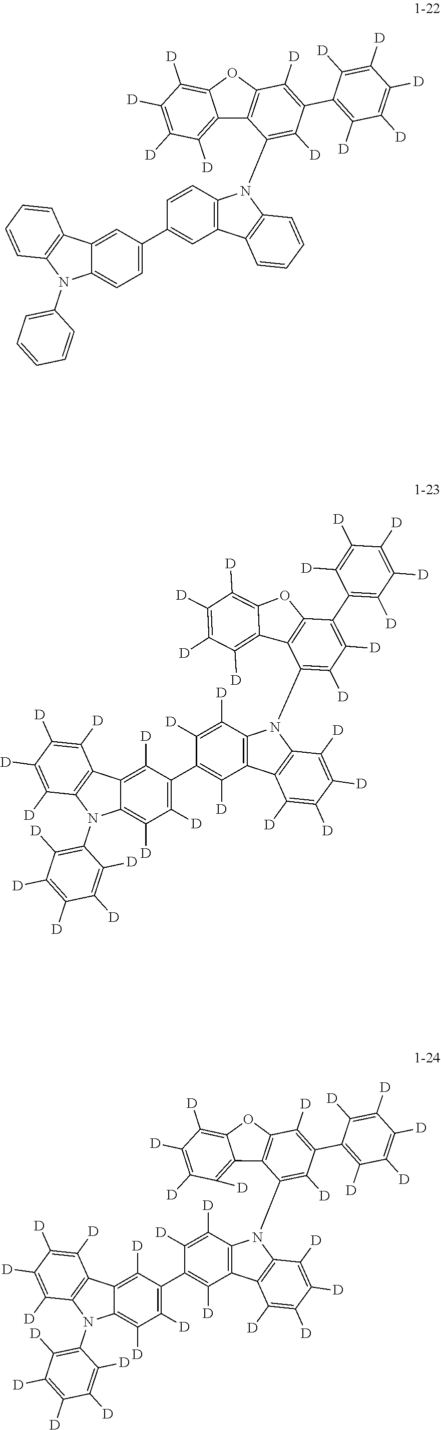

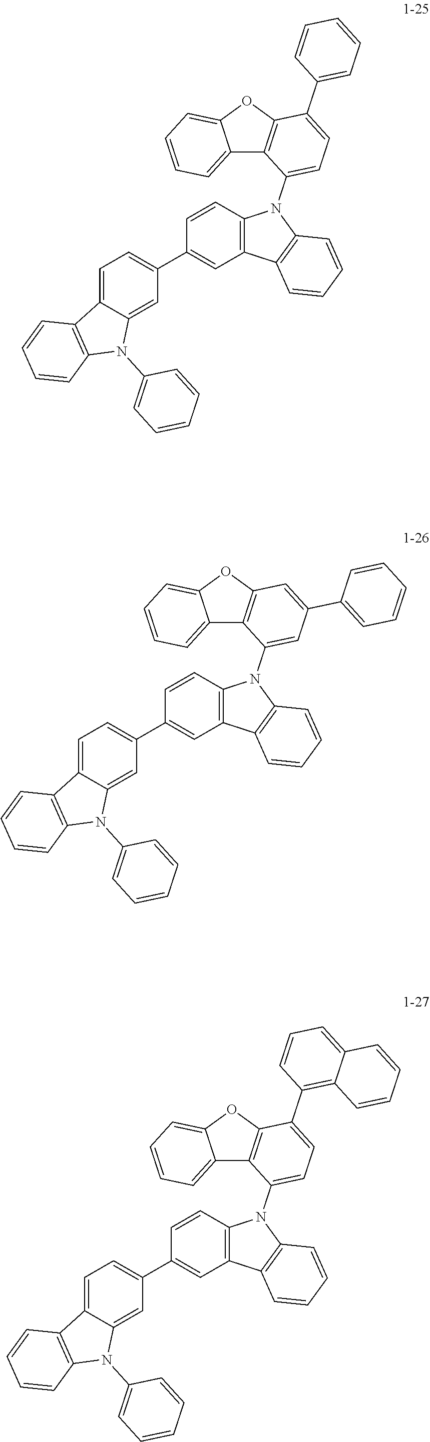 [SMILES: c1ccc(-c2ccc(-n3c4ccccc4c4cc(-c5ccc6c7ccccc7n(-c7ccccc7)c6c5)ccc43)c3c2oc2ccccc23)cc1 ; c1ccc(-c2cc(-n3c4ccccc4c4cc(-c5ccc6c7ccccc7n(-c7ccccc7)c6c5)ccc43)c3c(c2)oc2ccccc23)cc1 ; c1ccc(-n2c3ccccc3c3ccc(-c4ccc5c(c4)c4ccccc4n5-c4ccc(-c5cccc6ccccc56)c5oc6ccccc6c45)cc32)cc1]

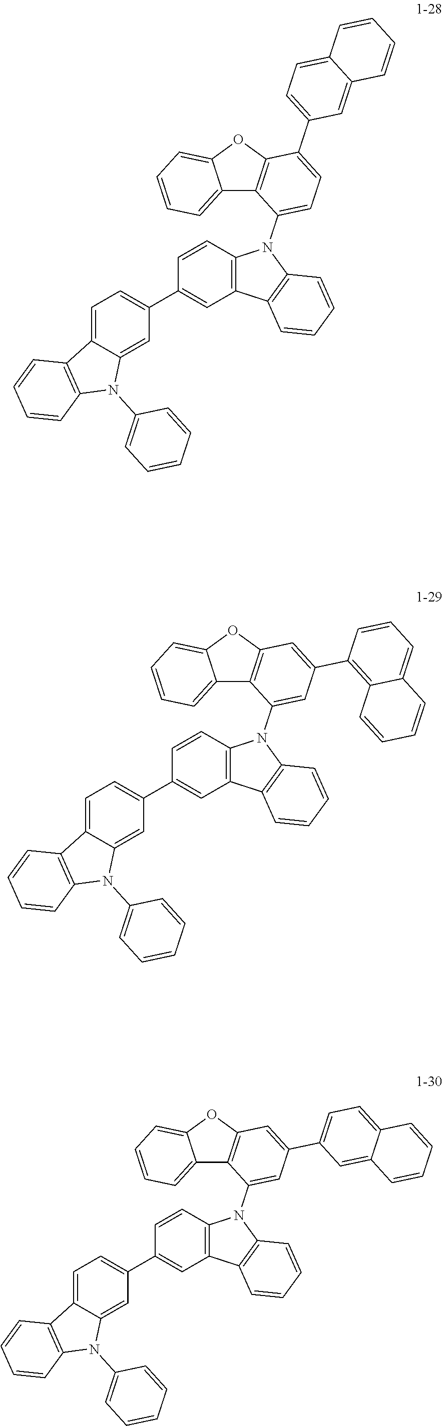 [SMILES: c1ccc(-n2c3ccccc3c3ccc(-c4ccc5c(c4)c4ccccc4n5-c4ccc(-c5ccc6ccccc6c5)c5oc6ccccc6c45)cc32)cc1 ; c1ccc(-n2c3ccccc3c3ccc(-c4ccc5c(c4)c4ccccc4n5-c4cc(-c5cccc6ccccc56)cc5oc6ccccc6c45)cc32)cc1 ; c1ccc(-n2c3ccccc3c3ccc(-c4ccc5c(c4)c4ccccc4n5-c4cc(-c5ccc6ccccc6c5)cc5oc6ccccc6c45)cc32)cc1]

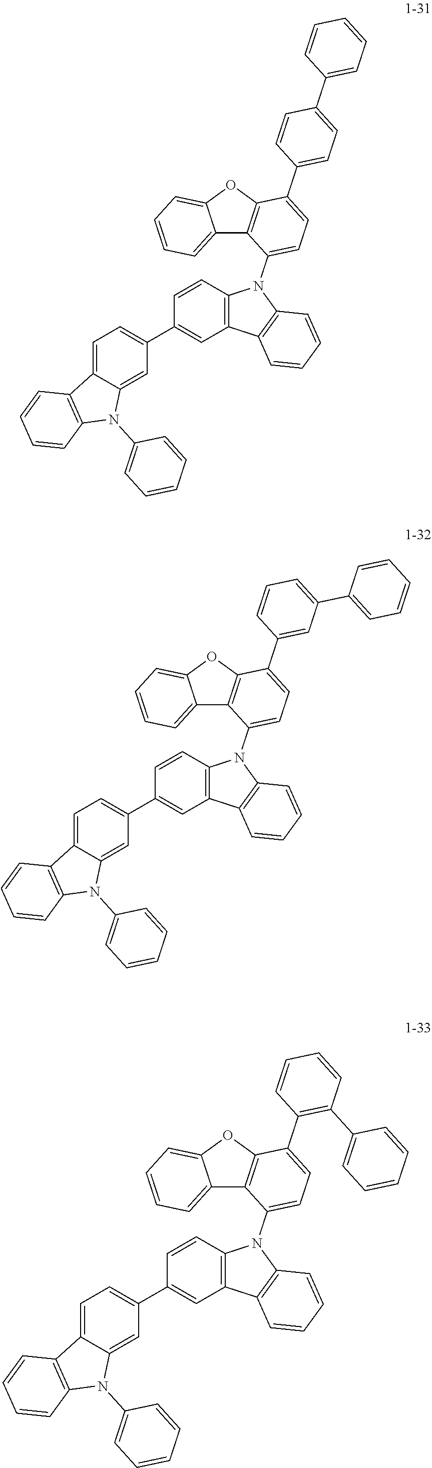 [SMILES: c1ccc(-c2ccc(-c3ccc(-n4c5ccccc5c5cc(-c6ccc7c8ccccc8n(-c8ccccc8)c7c6)ccc54)c4c3oc3ccccc34)cc2)cc1 ; c1ccc(-c2cccc(-c3ccc(-n4c5ccccc5c5cc(-c6ccc7c8ccccc8n(-c8ccccc8)c7c6)ccc54)c4c3oc3ccccc34)c2)cc1 ; c1ccc(-c2ccccc2-c2ccc(-n3c4ccccc4c4cc(-c5ccc6c7ccccc7n(-c7ccccc7)c6c5)ccc43)c3c2oc2ccccc23)cc1]

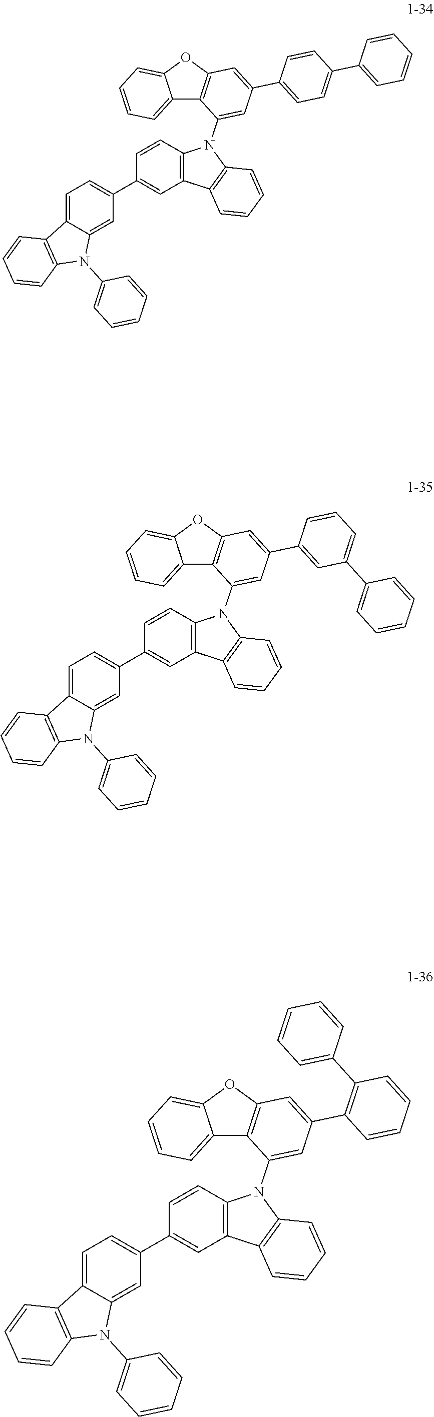 [SMILES: c1ccc(-c2ccc(-c3cc(-n4c5ccccc5c5cc(-c6ccc7c8ccccc8n(-c8ccccc8)c7c6)ccc54)c4c(c3)oc3ccccc34)cc2)cc1 ; c1ccc(-c2cccc(-c3cc(-n4c5ccccc5c5cc(-c6ccc7c8ccccc8n(-c8ccccc8)c7c6)ccc54)c4c(c3)oc3ccccc34)c2)cc1 ; c1ccc(-c2ccccc2-c2cc(-n3c4ccccc4c4cc(-c5ccc6c7ccccc7n(-c7ccccc7)c6c5)ccc43)c3c(c2)oc2ccccc23)cc1]

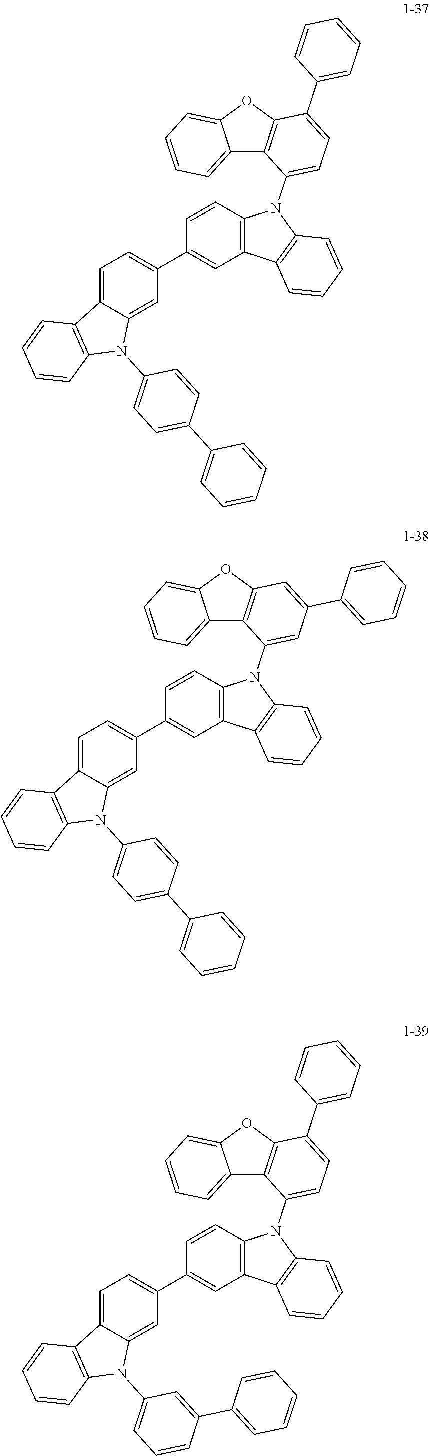 [SMILES: c1ccc(-c2ccc(-n3c4ccccc4c4ccc(-c5ccc6c(c5)c5ccccc5n6-c5ccc(-c6ccccc6)c6oc7ccccc7c56)cc43)cc2)cc1 ; c1ccc(-c2ccc(-n3c4ccccc4c4ccc(-c5ccc6c(c5)c5ccccc5n6-c5cc(-c6ccccc6)cc6oc7ccccc7c56)cc43)cc2)cc1 ; c1ccc(-c2cccc(-n3c4ccccc4c4ccc(-c5ccc6c(c5)c5ccccc5n6-c5ccc(-c6ccccc6)c6oc7ccccc7c56)cc43)c2)cc1]

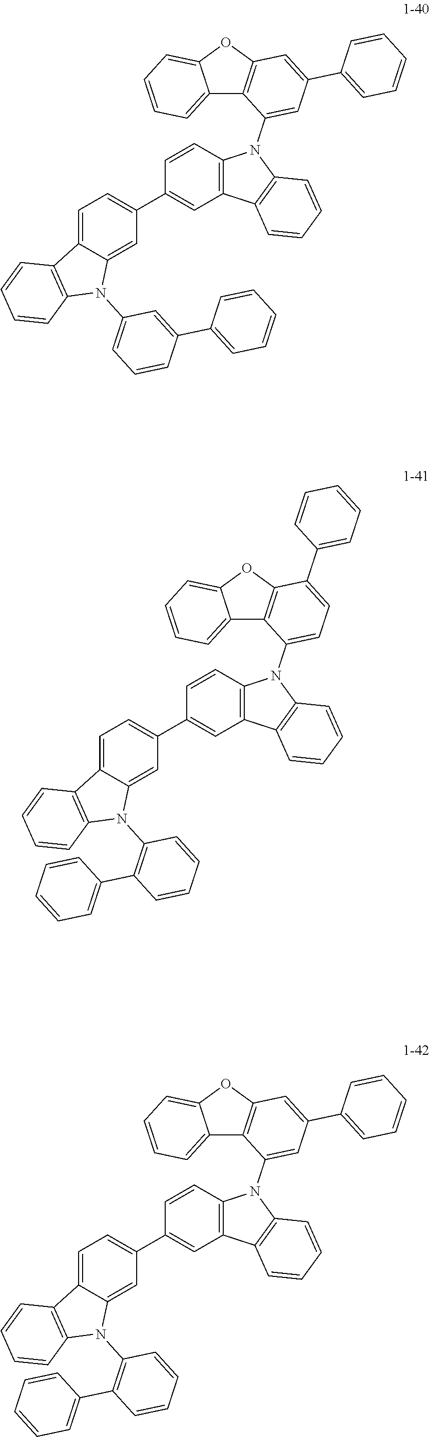 [SMILES: c1ccc(-c2cccc(-n3c4ccccc4c4ccc(-c5ccc6c(c5)c5ccccc5n6-c5cc(-c6ccccc6)cc6oc7ccccc7c56)cc43)c2)cc1 ; c1ccc(-c2ccccc2-n2c3ccccc3c3ccc(-c4ccc5c(c4)c4ccccc4n5-c4ccc(-c5ccccc5)c5oc6ccccc6c45)cc32)cc1 ; c1ccc(-c2cc(-n3c4ccccc4c4cc(-c5ccc6c7ccccc7n(-c7ccccc7-c7ccccc7)c6c5)ccc43)c3c(c2)oc2ccccc23)cc1]

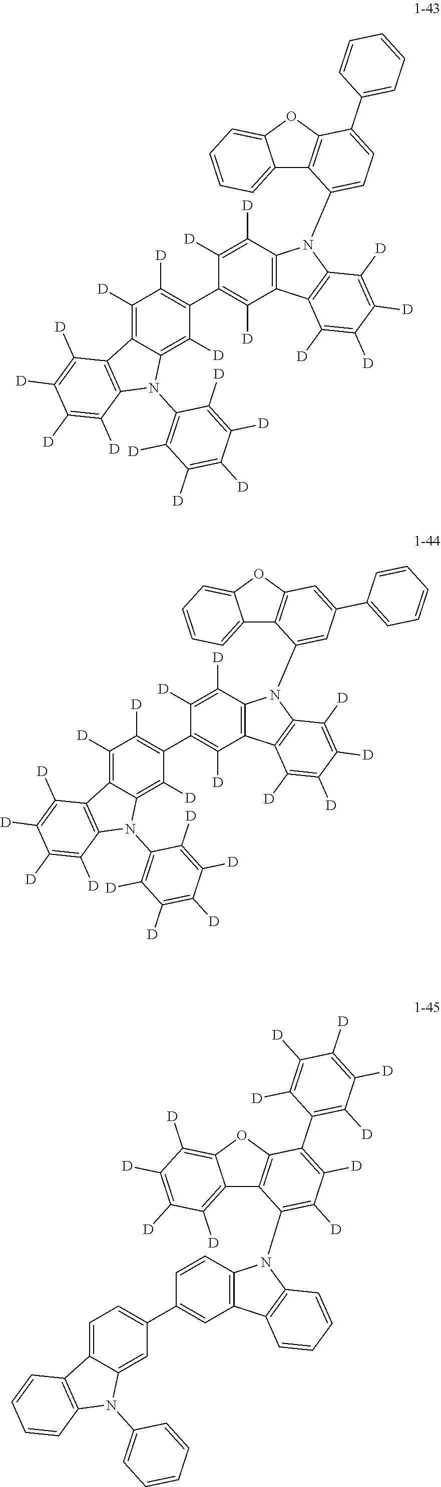

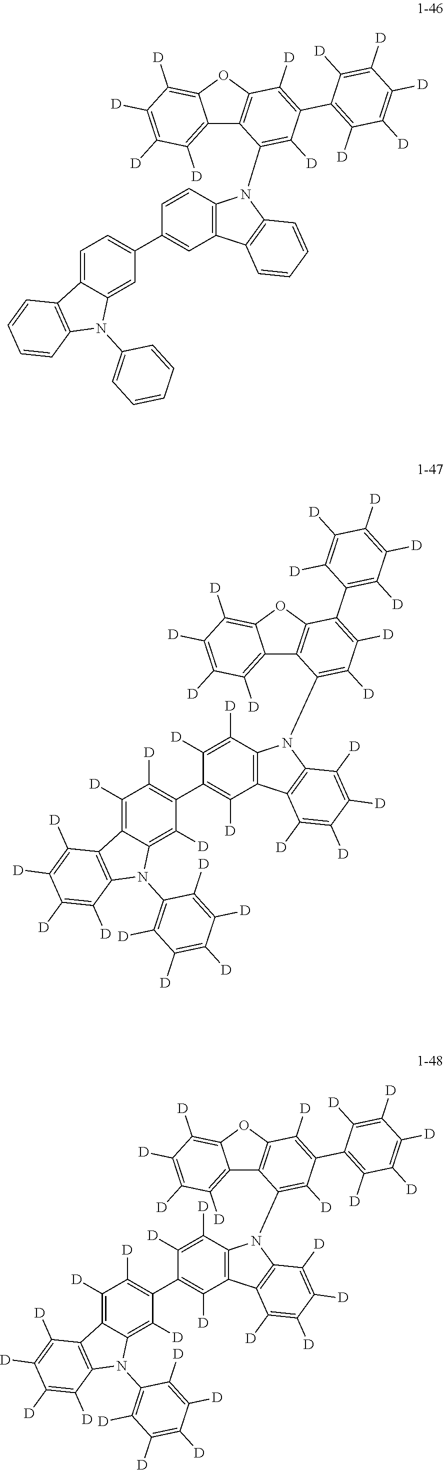

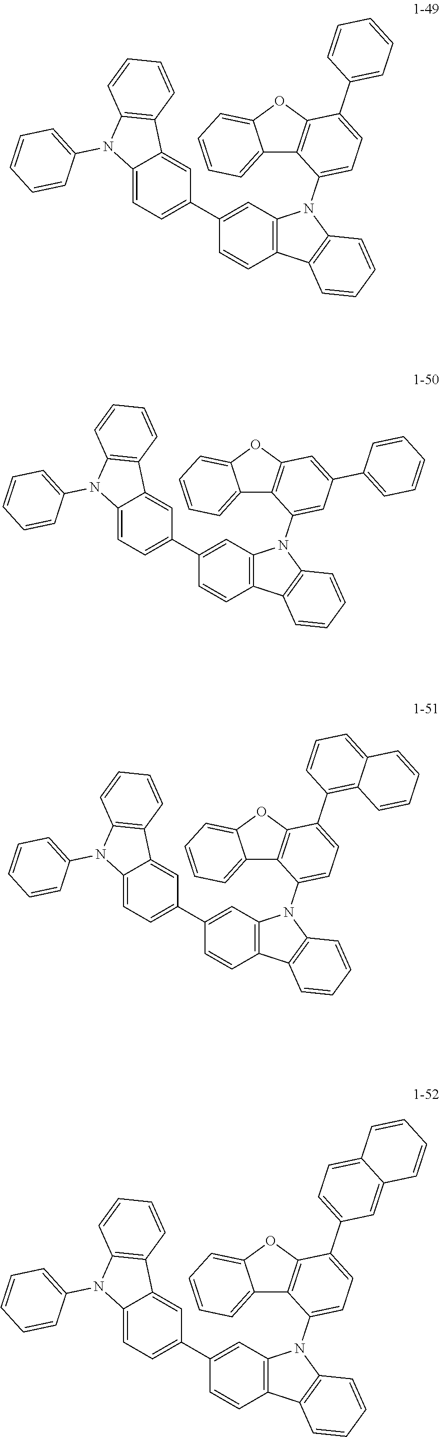 [SMILES: c1ccc(-c2ccc(-n3c4ccccc4c4ccc(-c5ccc6c(c5)c5ccccc5n6-c5ccccc5)cc43)c3c2oc2ccccc23)cc1 ; c1ccc(-c2cc(-n3c4ccccc4c4ccc(-c5ccc6c(c5)c5ccccc5n6-c5ccccc5)cc43)c3c(c2)oc2ccccc23)cc1 ; c1ccc(-n2c3ccccc3c3cc(-c4ccc5c6ccccc6n(-c6ccc(-c7cccc8ccccc78)c7oc8ccccc8c67)c5c4)ccc32)cc1 ; c1ccc(-n2c3ccccc3c3cc(-c4ccc5c6ccccc6n(-c6ccc(-c7ccc8ccccc8c7)c7oc8ccccc8c67)c5c4)ccc32)cc1]

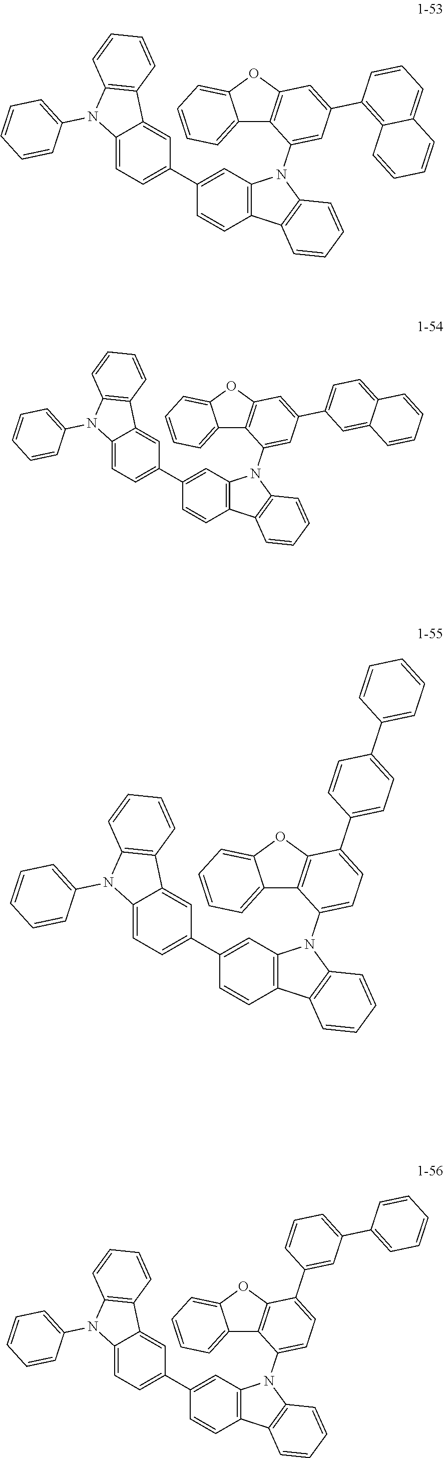 [SMILES: c1ccc(-n2c3ccccc3c3cc(-c4ccc5c6ccccc6n(-c6cc(-c7cccc8ccccc78)cc7oc8ccccc8c67)c5c4)ccc32)cc1 ; c1ccc(-n2c3ccccc3c3cc(-c4ccc5c6ccccc6n(-c6cc(-c7ccc8ccccc8c7)cc7oc8ccccc8c67)c5c4)ccc32)cc1 ; c1ccc(-c2ccc(-c3ccc(-n4c5ccccc5c5ccc(-c6ccc7c(c6)c6ccccc6n7-c6ccccc6)cc54)c4c3oc3ccccc34)cc2)cc1 ; c1ccc(-c2cccc(-c3ccc(-n4c5ccccc5c5ccc(-c6ccc7c(c6)c6ccccc6n7-c6ccccc6)cc54)c4c3oc3ccccc34)c2)cc1]

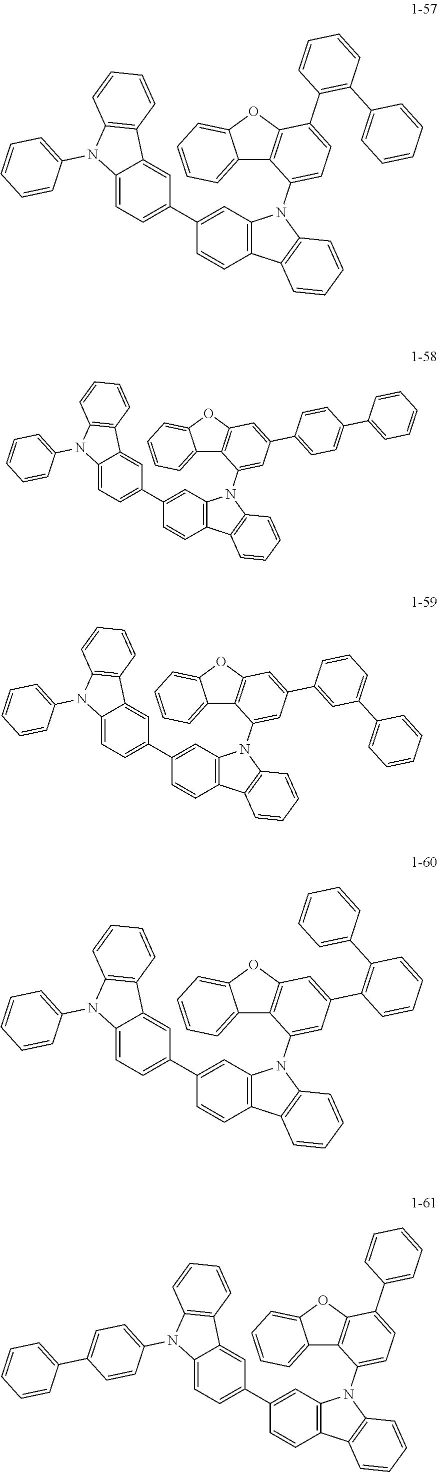 [SMILES: c1ccc(-c2ccccc2-c2ccc(-n3c4ccccc4c4ccc(-c5ccc6c(c5)c5ccccc5n6-c5ccccc5)cc43)c3c2oc2ccccc23)cc1 ; c1ccc(-c2ccc(-c3cc(-n4c5ccccc5c5ccc(-c6ccc7c(c6)c6ccccc6n7-c6ccccc6)cc54)c4c(c3)oc3ccccc34)cc2)cc1 ; c1ccc(-c2cccc(-c3cc(-n4c5ccccc5c5ccc(-c6ccc7c(c6)c6ccccc6n7-c6ccccc6)cc54)c4c(c3)oc3ccccc34)c2)cc1 ; c1ccc(-c2ccccc2-c2cc(-n3c4ccccc4c4ccc(-c5ccc6c(c5)c5ccccc5n6-c5ccccc5)cc43)c3c(c2)oc2ccccc23)cc1 ; c1ccc(-c2ccc(-n3c4ccccc4c4cc(-c5ccc6c7ccccc7n(-c7ccc(-c8ccccc8)c8oc9ccccc9c78)c6c5)ccc43)cc2)cc1]

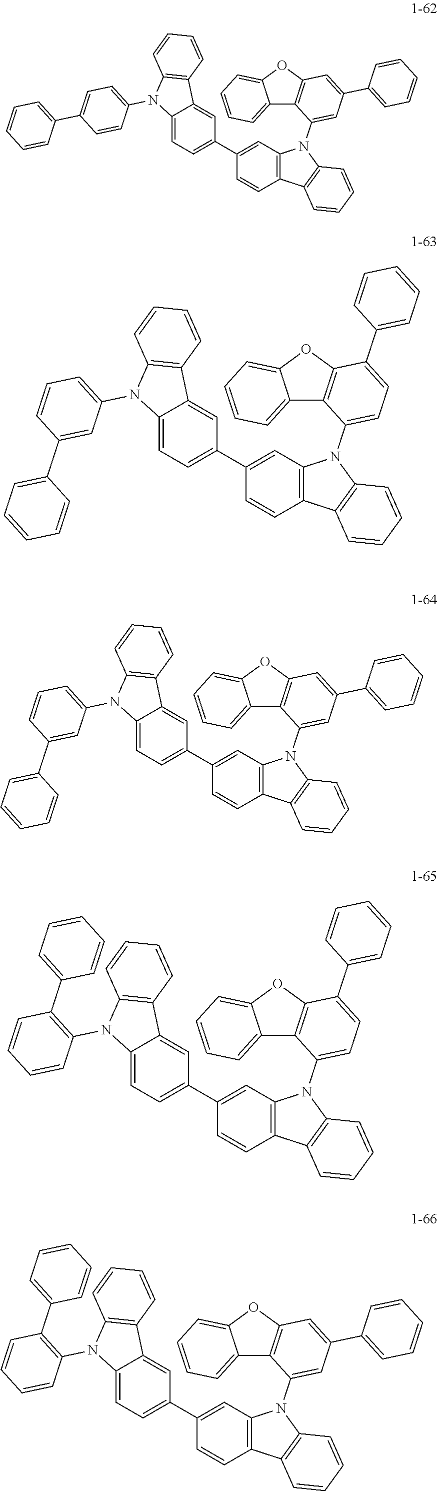 [SMILES: c1ccc(-c2ccc(-n3c4ccccc4c4cc(-c5ccc6c7ccccc7n(-c7cc(-c8ccccc8)cc8oc9ccccc9c78)c6c5)ccc43)cc2)cc1 ; c1ccc(-c2cccc(-n3c4ccccc4c4cc(-c5ccc6c7ccccc7n(-c7ccc(-c8ccccc8)c8oc9ccccc9c78)c6c5)ccc43)c2)cc1 ; c1ccc(-c2cccc(-n3c4ccccc4c4cc(-c5ccc6c7ccccc7n(-c7cc(-c8ccccc8)cc8oc9ccccc9c78)c6c5)ccc43)c2)cc1 ; c1ccc(-c2ccccc2-n2c3ccccc3c3cc(-c4ccc5c6ccccc6n(-c6ccc(-c7ccccc7)c7oc8ccccc8c67)c5c4)ccc32)cc1 ; c1ccc(-c2cc(-n3c4ccccc4c4ccc(-c5ccc6c(c5)c5ccccc5n6-c5ccccc5-c5ccccc5)cc43)c3c(c2)oc2ccccc23)cc1]

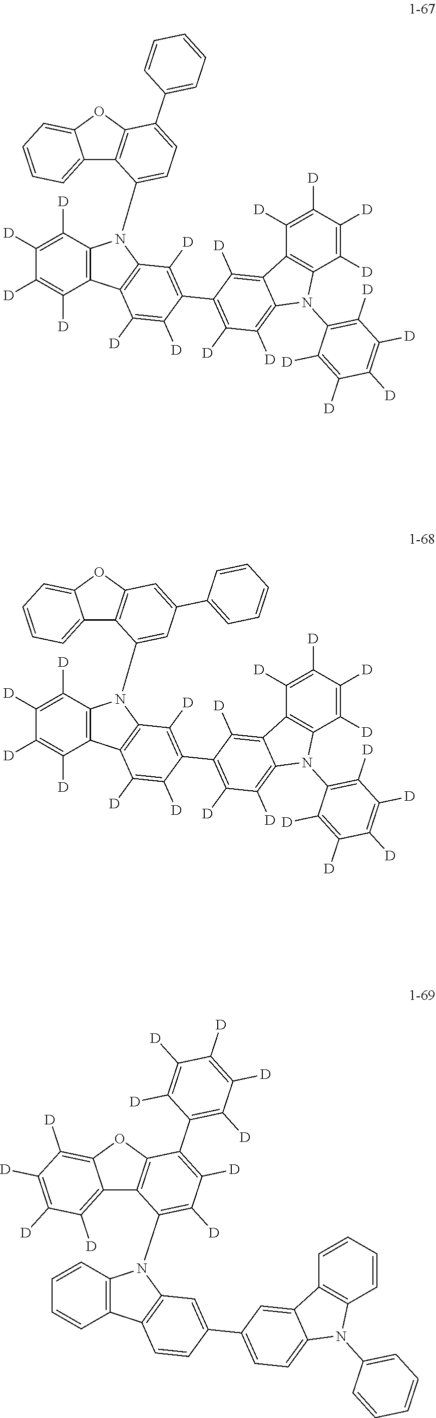

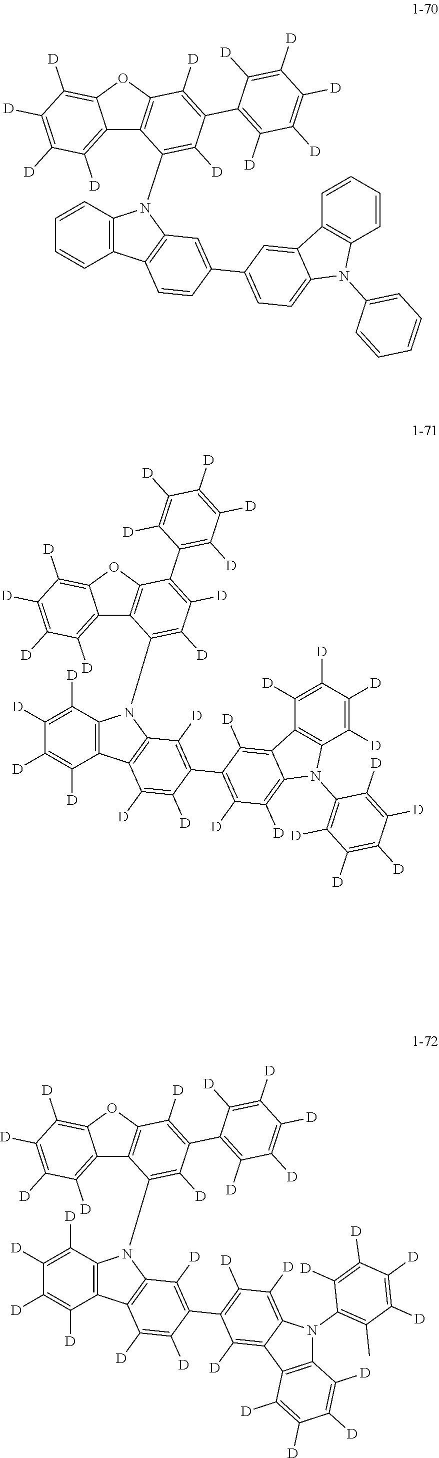

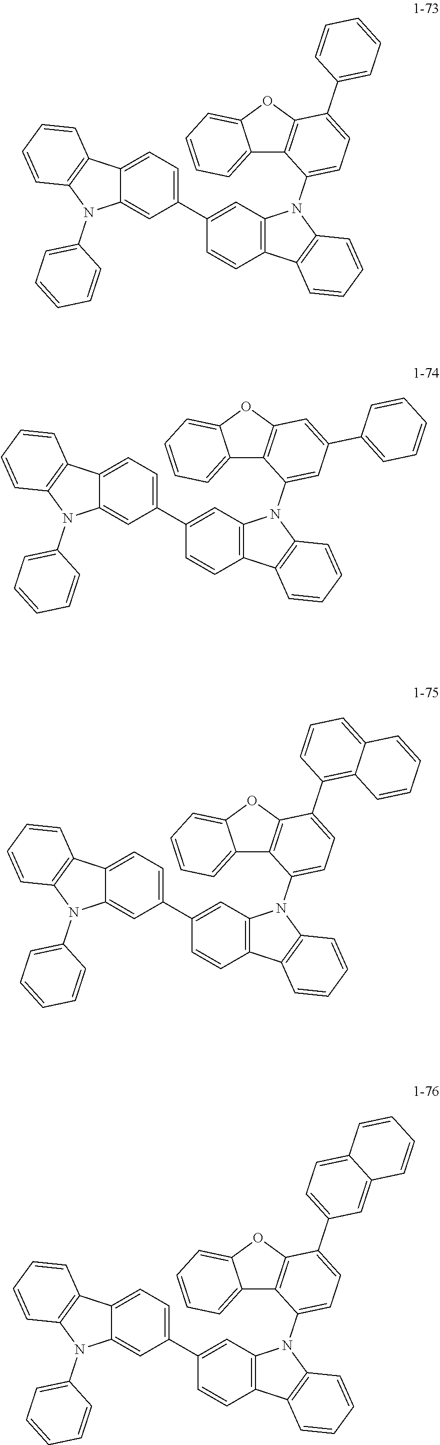 [SMILES: c1ccc(-c2ccc(-n3c4ccccc4c4ccc(-c5ccc6c7ccccc7n(-c7ccccc7)c6c5)cc43)c3c2oc2ccccc23)cc1 ; c1ccc(-c2cc(-n3c4ccccc4c4ccc(-c5ccc6c7ccccc7n(-c7ccccc7)c6c5)cc43)c3c(c2)oc2ccccc23)cc1 ; c1ccc(-n2c3ccccc3c3ccc(-c4ccc5c6ccccc6n(-c6ccc(-c7cccc8ccccc78)c7oc8ccccc8c67)c5c4)cc32)cc1 ; c1ccc(-n2c3ccccc3c3ccc(-c4ccc5c6ccccc6n(-c6ccc(-c7ccc8ccccc8c7)c7oc8ccccc8c67)c5c4)cc32)cc1]

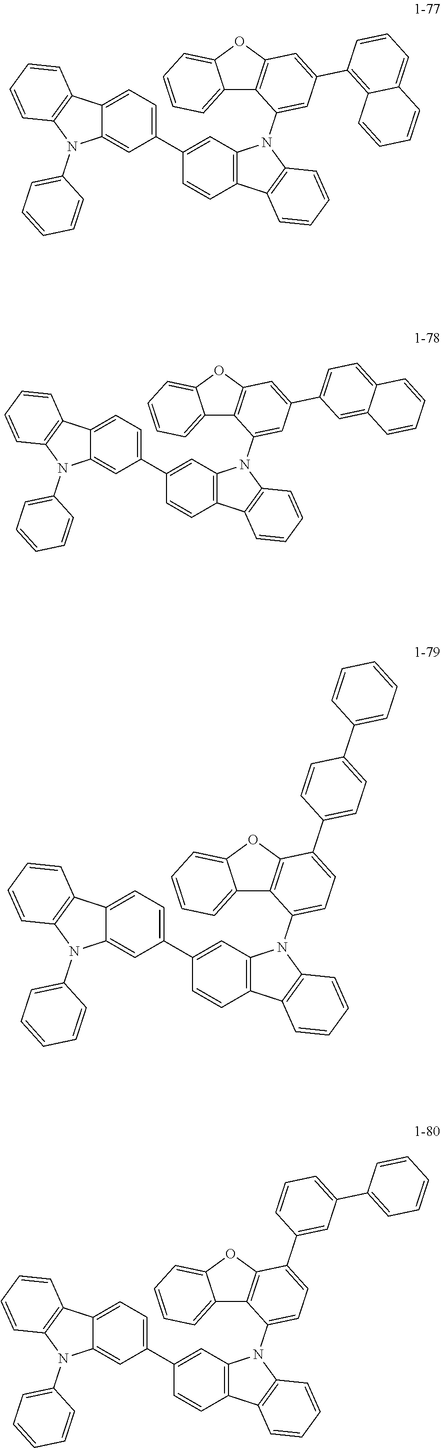 [SMILES: c1ccc(-n2c3ccccc3c3ccc(-c4ccc5c6ccccc6n(-c6cc(-c7cccc8ccccc78)cc7oc8ccccc8c67)c5c4)cc32)cc1 ; c1ccc(-n2c3ccccc3c3ccc(-c4ccc5c6ccccc6n(-c6cc(-c7ccc8ccccc8c7)cc7oc8ccccc8c67)c5c4)cc32)cc1 ; c1ccc(-c2ccc(-c3ccc(-n4c5ccccc5c5ccc(-c6ccc7c8ccccc8n(-c8ccccc8)c7c6)cc54)c4c3oc3ccccc34)cc2)cc1 ; c1ccc(-c2cccc(-c3ccc(-n4c5ccccc5c5ccc(-c6ccc7c8ccccc8n(-c8ccccc8)c7c6)cc54)c4c3oc3ccccc34)c2)cc1]

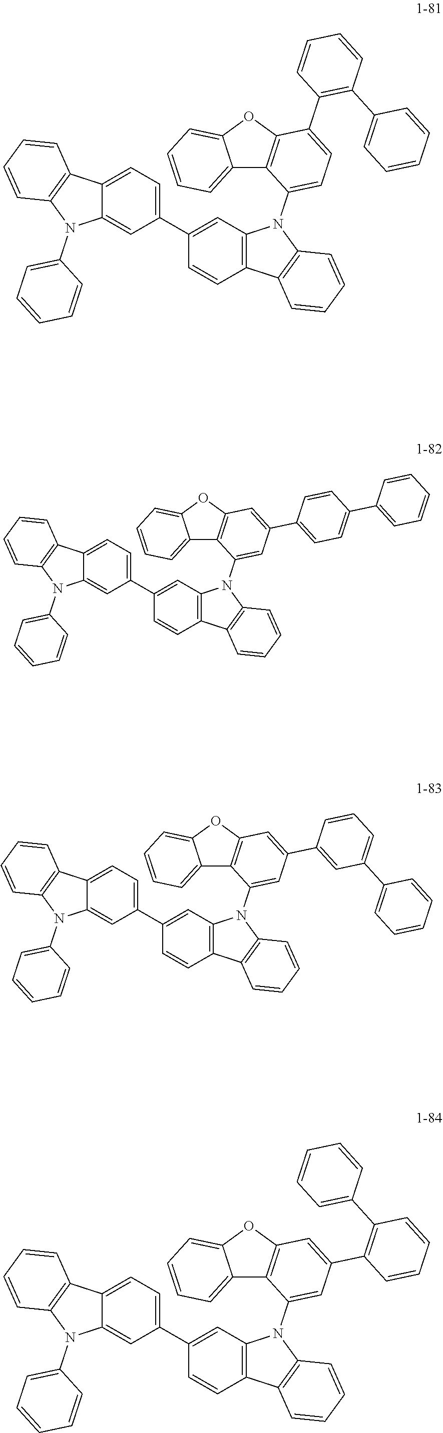 [SMILES: c1ccc(-c2ccccc2-c2ccc(-n3c4ccccc4c4ccc(-c5ccc6c7ccccc7n(-c7ccccc7)c6c5)cc43)c3c2oc2ccccc23)cc1 ; c1ccc(-c2ccc(-c3cc(-n4c5ccccc5c5ccc(-c6ccc7c8ccccc8n(-c8ccccc8)c7c6)cc54)c4c(c3)oc3ccccc34)cc2)cc1 ; c1ccc(-c2cccc(-c3cc(-n4c5ccccc5c5ccc(-c6ccc7c8ccccc8n(-c8ccccc8)c7c6)cc54)c4c(c3)oc3ccccc34)c2)cc1 ; c1ccc(-c2ccccc2-c2cc(-n3c4ccccc4c4ccc(-c5ccc6c7ccccc7n(-c7ccccc7)c6c5)cc43)c3c(c2)oc2ccccc23)cc1]

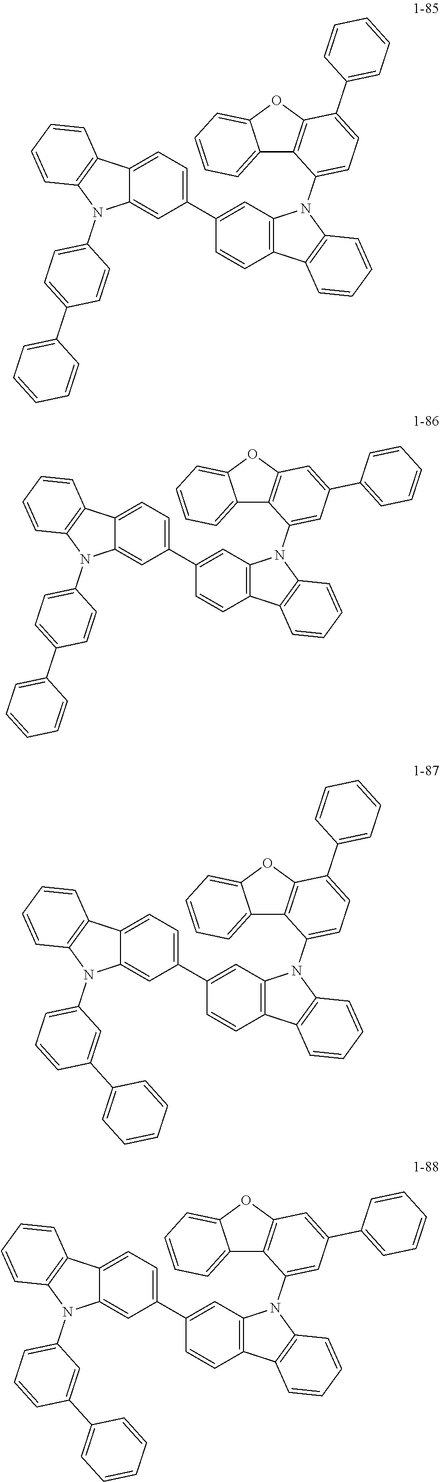 [SMILES: c1ccc(-c2ccc(-n3c4ccccc4c4ccc(-c5ccc6c7ccccc7n(-c7ccc(-c8ccccc8)c8oc9ccccc9c78)c6c5)cc43)cc2)cc1 ; c1ccc(-c2ccc(-n3c4ccccc4c4ccc(-c5ccc6c7ccccc7n(-c7cc(-c8ccccc8)cc8oc9ccccc9c78)c6c5)cc43)cc2)cc1 ; c1ccc(-c2cccc(-n3c4ccccc4c4ccc(-c5ccc6c7ccccc7n(-c7ccc(-c8ccccc8)c8oc9ccccc9c78)c6c5)cc43)c2)cc1 ; c1ccc(-c2cccc(-n3c4ccccc4c4ccc(-c5ccc6c7ccccc7n(-c7cc(-c8ccccc8)cc8oc9ccccc9c78)c6c5)cc43)c2)cc1]

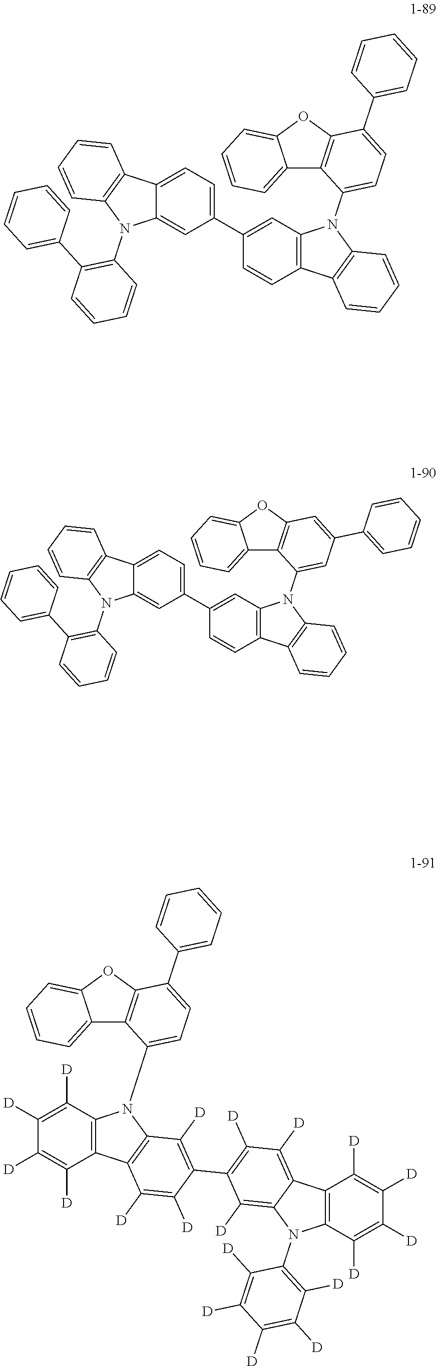 [SMILES: c1ccc(-c2ccccc2-n2c3ccccc3c3ccc(-c4ccc5c6ccccc6n(-c6ccc(-c7ccccc7)c7oc8ccccc8c67)c5c4)cc32)cc1 ; c1ccc(-c2cc(-n3c4ccccc4c4ccc(-c5ccc6c7ccccc7n(-c7ccccc7-c7ccccc7)c6c5)cc43)c3c(c2)oc2ccccc23)cc1 ; [2H]c1c([2H])c([2H])c(-n2c3c([2H])c([2H])c([2H])c([2H])c3c3c([2H])c([2H])c(-c4c([2H])c([2H])c5c6c([2H])c([2H])c([2H])c([2H])c6n(-c6ccc(-c7ccccc7)c7oc8ccccc8c67)c5c4[2H])c([2H])c32)c([2H])c1[2H]]

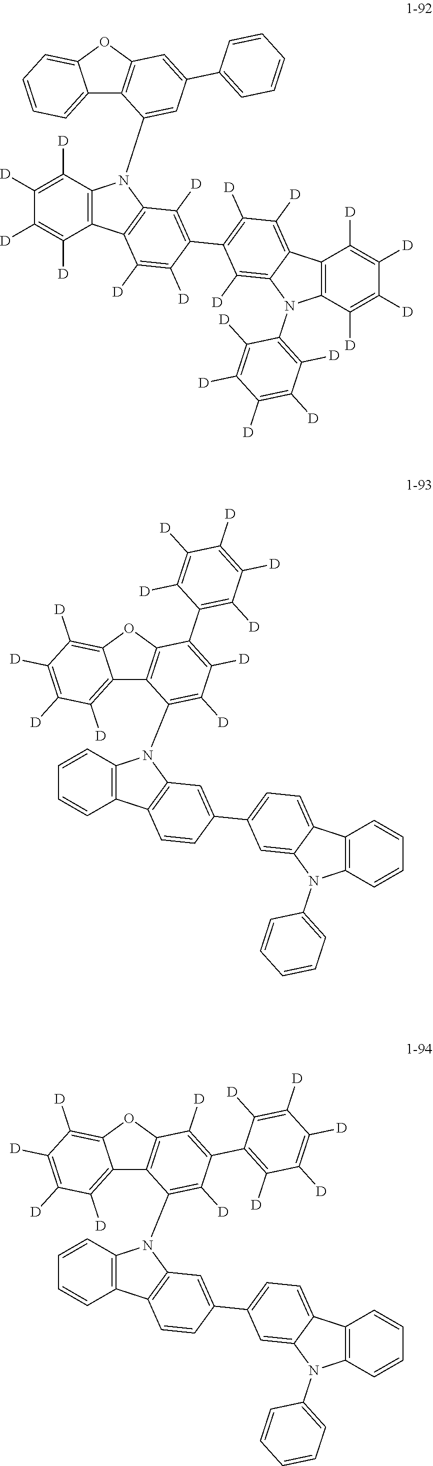 [SMILES: [2H]c1c([2H])c([2H])c(-n2c3c([2H])c([2H])c([2H])c([2H])c3c3c([2H])c([2H])c(-c4c([2H])c([2H])c5c6c([2H])c([2H])c([2H])c([2H])c6n(-c6cc(-c7ccccc7)cc7oc8ccccc8c67)c5c4[2H])c([2H])c32)c([2H])c1[2H] ; [2H]c1c([2H])c([2H])c(-c2c([2H])c([2H])c(-n3c4ccccc4c4ccc(-c5ccc6c7ccccc7n(-c7ccccc7)c6c5)cc43)c3c2oc2c([2H])c([2H])c([2H])c([2H])c23)c([2H])c1[2H] ; [2H]c1c([2H])c([2H])c(-c2c([2H])c(-n3c4ccccc4c4ccc(-c5ccc6c7ccccc7n(-c7ccccc7)c6c5)cc43)c3c(oc4c([2H])c([2H])c([2H])c([2H])c43)c2[2H])c([2H])c1[2H]]

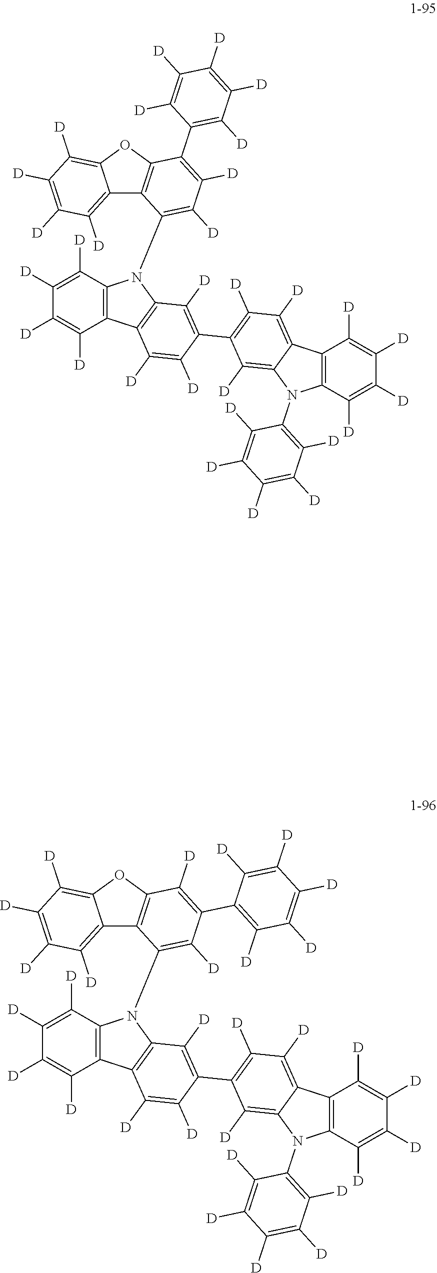

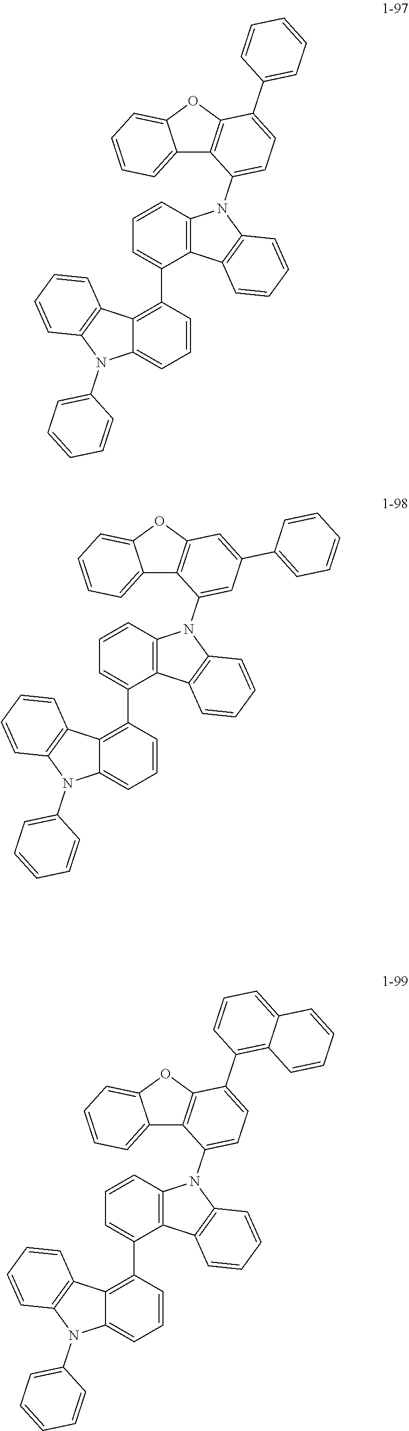 [SMILES: c1ccc(-c2ccc(-n3c4ccccc4c4c(-c5cccc6c5c5ccccc5n6-c5ccccc5)cccc43)c3c2oc2ccccc23)cc1 ; c1ccc(-c2cc(-n3c4ccccc4c4c(-c5cccc6c5c5ccccc5n6-c5ccccc5)cccc43)c3c(c2)oc2ccccc23)cc1 ; c1ccc(-n2c3ccccc3c3c(-c4cccc5c4c4ccccc4n5-c4ccc(-c5cccc6ccccc56)c5oc6ccccc6c45)cccc32)cc1]

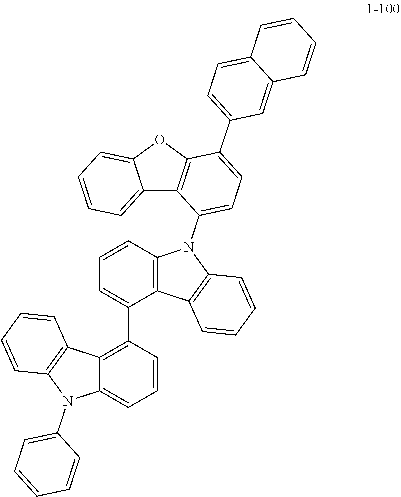 [SMILES: c1ccc(-n2c3ccccc3c3c(-c4cccc5c4c4ccccc4n5-c4ccc(-c5ccc6ccccc6c5)c5oc6ccccc6c45)cccc32)cc1]

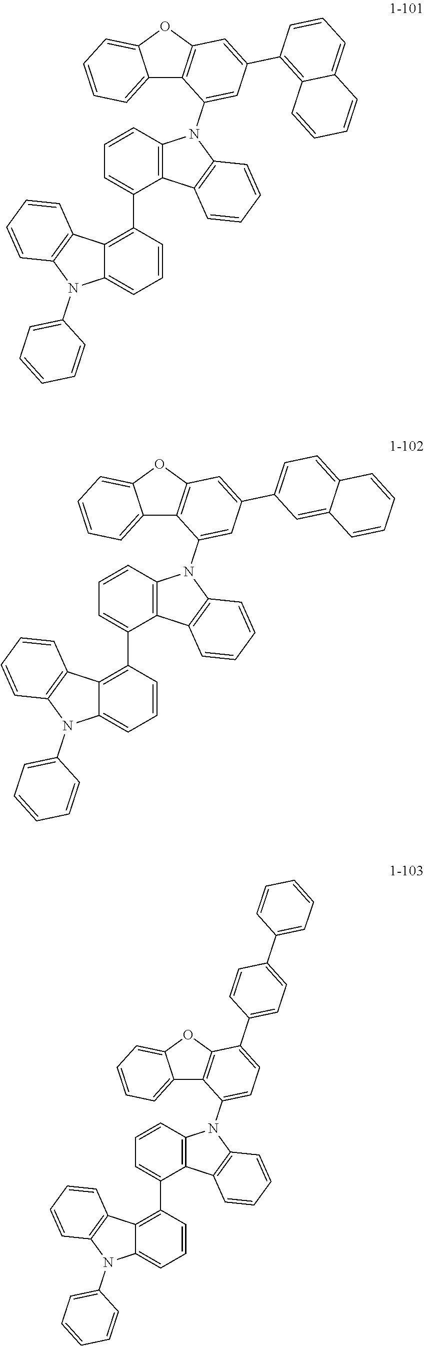 [SMILES: c1ccc(-n2c3ccccc3c3c(-c4cccc5c4c4ccccc4n5-c4cc(-c5cccc6ccccc56)cc5oc6ccccc6c45)cccc32)cc1 ; c1ccc(-n2c3ccccc3c3c(-c4cccc5c4c4ccccc4n5-c4cc(-c5ccc6ccccc6c5)cc5oc6ccccc6c45)cccc32)cc1 ; c1ccc(-c2ccc(-c3ccc(-n4c5ccccc5c5c(-c6cccc7c6c6ccccc6n7-c6ccccc6)cccc54)c4c3oc3ccccc34)cc2)cc1]

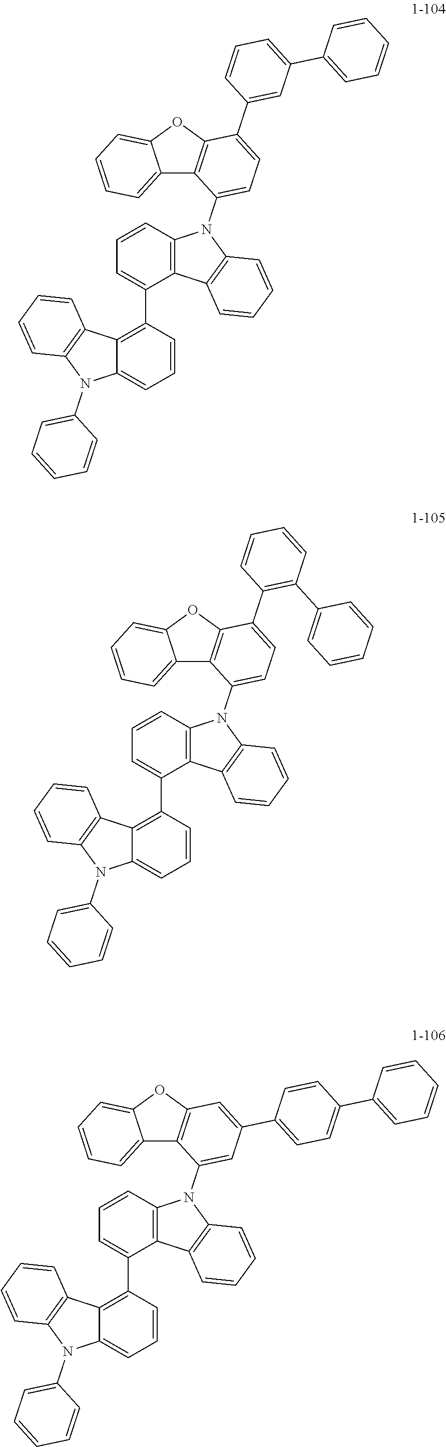 [SMILES: c1ccc(-c2cccc(-c3ccc(-n4c5ccccc5c5c(-c6cccc7c6c6ccccc6n7-c6ccccc6)cccc54)c4c3oc3ccccc34)c2)cc1 ; c1ccc(-c2ccccc2-c2ccc(-n3c4ccccc4c4c(-c5cccc6c5c5ccccc5n6-c5ccccc5)cccc43)c3c2oc2ccccc23)cc1 ; c1ccc(-c2ccc(-c3cc(-n4c5ccccc5c5c(-c6cccc7c6c6ccccc6n7-c6ccccc6)cccc54)c4c(c3)oc3ccccc34)cc2)cc1]

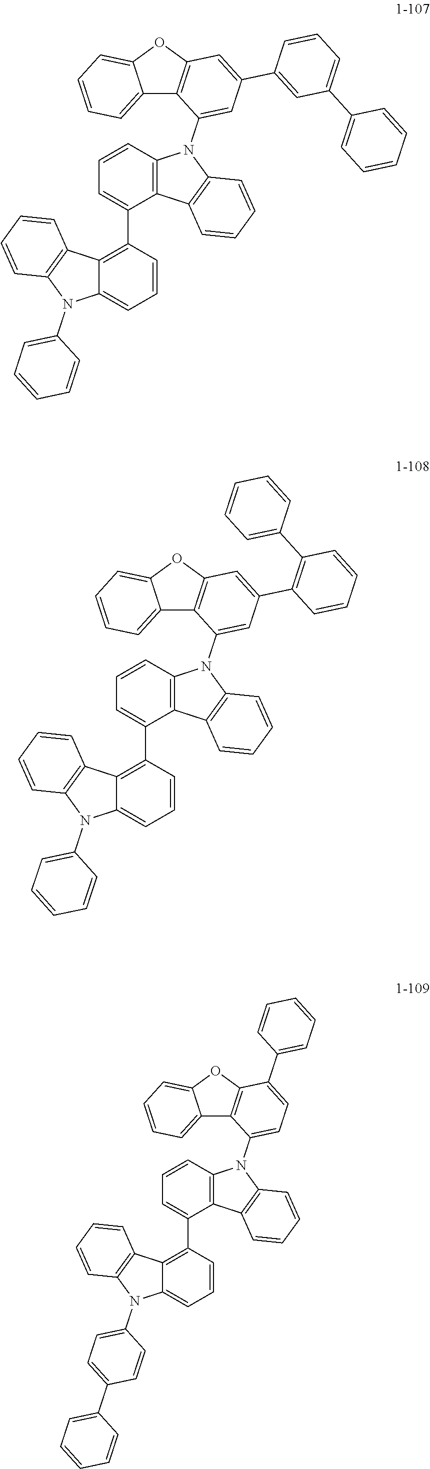 [SMILES: c1ccc(-c2cccc(-c3cc(-n4c5ccccc5c5c(-c6cccc7c6c6ccccc6n7-c6ccccc6)cccc54)c4c(c3)oc3ccccc34)c2)cc1 ; c1ccc(-c2ccccc2-c2cc(-n3c4ccccc4c4c(-c5cccc6c5c5ccccc5n6-c5ccccc5)cccc43)c3c(c2)oc2ccccc23)cc1 ; c1ccc(-c2ccc(-n3c4ccccc4c4c(-c5cccc6c5c5ccccc5n6-c5ccc(-c6ccccc6)c6oc7ccccc7c56)cccc43)cc2)cc1]

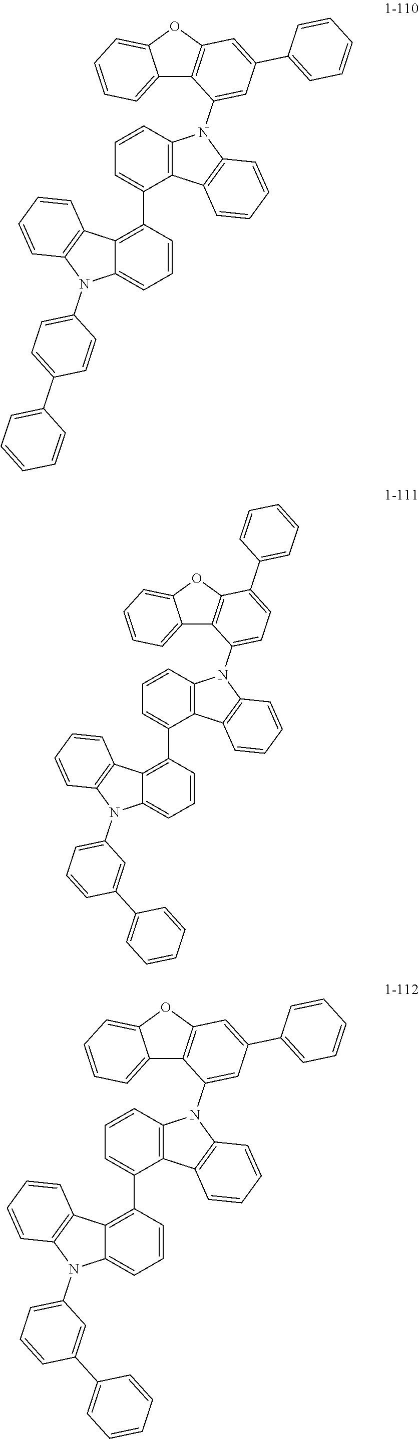 [SMILES: c1ccc(-c2ccc(-n3c4ccccc4c4c(-c5cccc6c5c5ccccc5n6-c5cc(-c6ccccc6)cc6oc7ccccc7c56)cccc43)cc2)cc1 ; c1ccc(-c2cccc(-n3c4ccccc4c4c(-c5cccc6c5c5ccccc5n6-c5ccc(-c6ccccc6)c6oc7ccccc7c56)cccc43)c2)cc1 ; c1ccc(-c2cccc(-n3c4ccccc4c4c(-c5cccc6c5c5ccccc5n6-c5cc(-c6ccccc6)cc6oc7ccccc7c56)cccc43)c2)cc1]

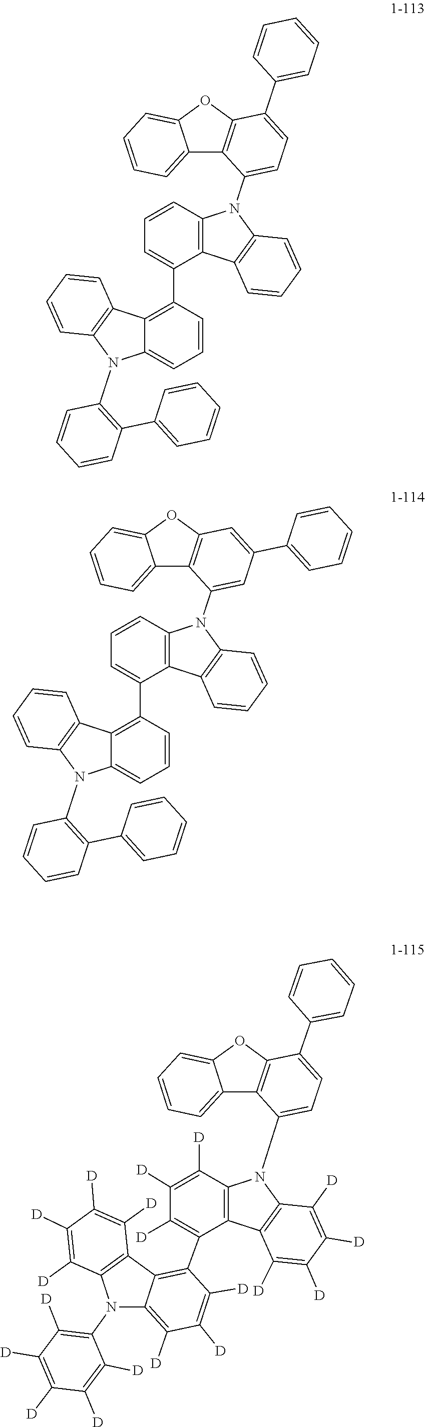 [SMILES: c1ccc(-c2ccccc2-n2c3ccccc3c3c(-c4cccc5c4c4ccccc4n5-c4ccc(-c5ccccc5)c5oc6ccccc6c45)cccc32)cc1 ; c1ccc(-c2cc(-n3c4ccccc4c4c(-c5cccc6c5c5ccccc5n6-c5ccccc5-c5ccccc5)cccc43)c3c(c2)oc2ccccc23)cc1 ; [2H]c1c([2H])c([2H])c(-n2c3c([2H])c([2H])c([2H])c([2H])c3c3c(-c4c([2H])c([2H])c([2H])c5c4c4c([2H])c([2H])c([2H])c([2H])c4n5-c4ccc(-c5ccccc5)c5oc6ccccc6c45)c([2H])c([2H])c([2H])c32)c([2H])c1[2H]]

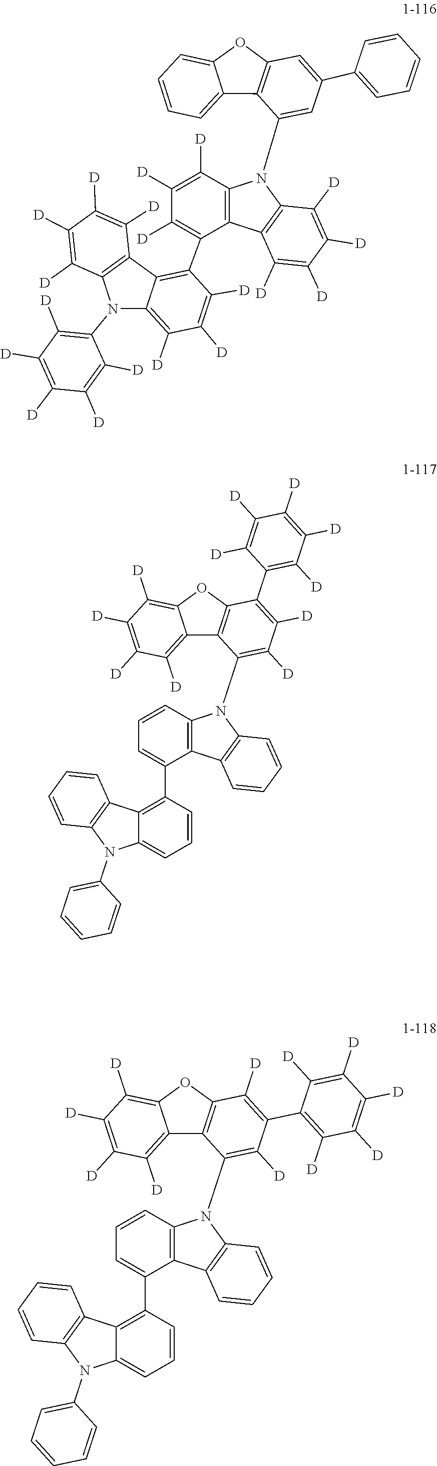 [SMILES: [2H]c1c([2H])c([2H])c(-n2c3c([2H])c([2H])c([2H])c([2H])c3c3c(-c4c([2H])c([2H])c([2H])c5c4c4c([2H])c([2H])c([2H])c([2H])c4n5-c4cc(-c5ccccc5)cc5oc6ccccc6c45)c([2H])c([2H])c([2H])c32)c([2H])c1[2H] ; [2H]c1c([2H])c([2H])c(-c2c([2H])c([2H])c(-n3c4ccccc4c4c(-c5cccc6c5c5ccccc5n6-c5ccccc5)cccc43)c3c2oc2c([2H])c([2H])c([2H])c([2H])c23)c([2H])c1[2H] ; [2H]c1c([2H])c([2H])c(-c2c([2H])c(-n3c4ccccc4c4c(-c5cccc6c5c5ccccc5n6-c5ccccc5)cccc43)c3c(oc4c([2H])c([2H])c([2H])c([2H])c43)c2[2H])c([2H])c1[2H]]

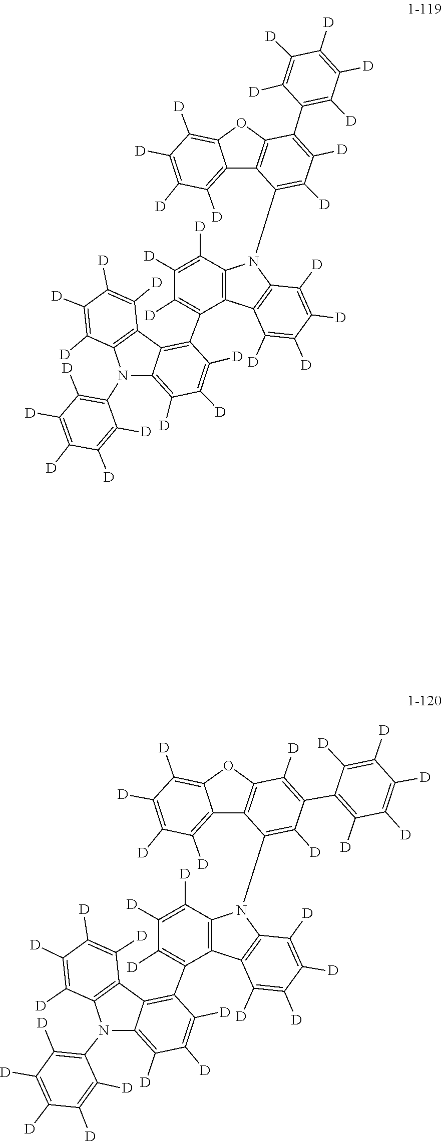

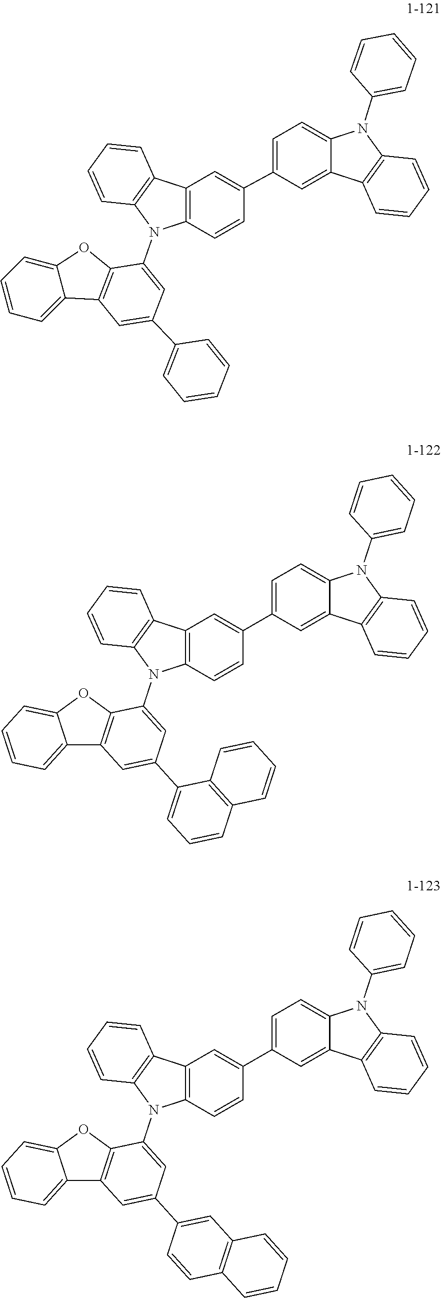 [SMILES: c1ccc(-c2cc(-n3c4ccccc4c4cc(-c5ccc6c(c5)c5ccccc5n6-c5ccccc5)ccc43)c3oc4ccccc4c3c2)cc1 ; c1ccc(-n2c3ccccc3c3cc(-c4ccc5c(c4)c4ccccc4n5-c4cc(-c5cccc6ccccc56)cc5c4oc4ccccc45)ccc32)cc1 ; c1ccc(-n2c3ccccc3c3cc(-c4ccc5c(c4)c4ccccc4n5-c4cc(-c5ccc6ccccc6c5)cc5c4oc4ccccc45)ccc32)cc1]

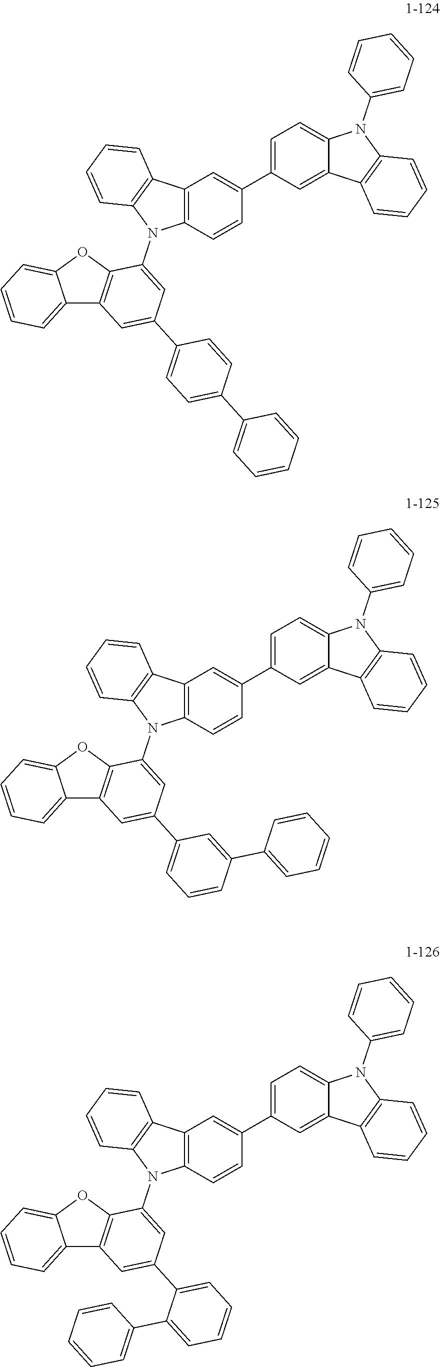 [SMILES: c1ccc(-c2ccc(-c3cc(-n4c5ccccc5c5cc(-c6ccc7c(c6)c6ccccc6n7-c6ccccc6)ccc54)c4oc5ccccc5c4c3)cc2)cc1 ; c1ccc(-c2cccc(-c3cc(-n4c5ccccc5c5cc(-c6ccc7c(c6)c6ccccc6n7-c6ccccc6)ccc54)c4oc5ccccc5c4c3)c2)cc1 ; c1ccc(-c2ccccc2-c2cc(-n3c4ccccc4c4cc(-c5ccc6c(c5)c5ccccc5n6-c5ccccc5)ccc43)c3oc4ccccc4c3c2)cc1]

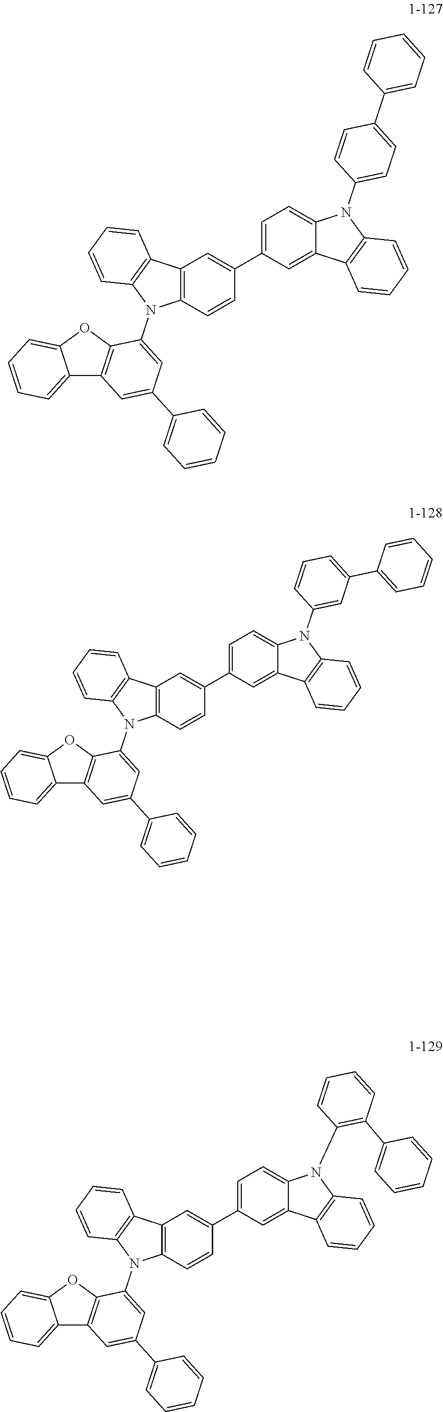 [SMILES: c1ccc(-c2ccc(-n3c4ccccc4c4cc(-c5ccc6c(c5)c5ccccc5n6-c5cc(-c6ccccc6)cc6c5oc5ccccc56)ccc43)cc2)cc1 ; c1ccc(-c2cccc(-n3c4ccccc4c4cc(-c5ccc6c(c5)c5ccccc5n6-c5cc(-c6ccccc6)cc6c5oc5ccccc56)ccc43)c2)cc1 ; c1ccc(-c2cc(-n3c4ccccc4c4cc(-c5ccc6c(c5)c5ccccc5n6-c5ccccc5-c5ccccc5)ccc43)c3oc4ccccc4c3c2)cc1]

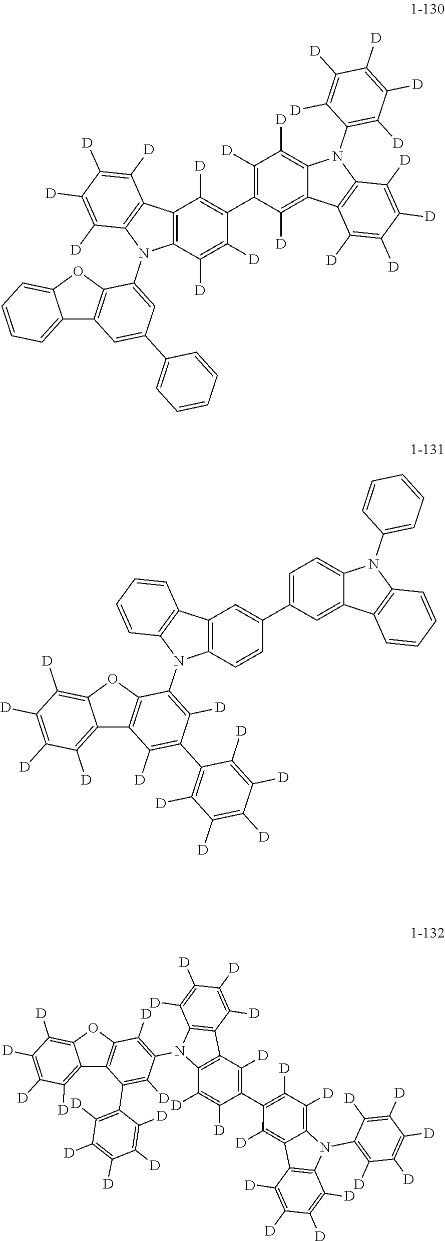

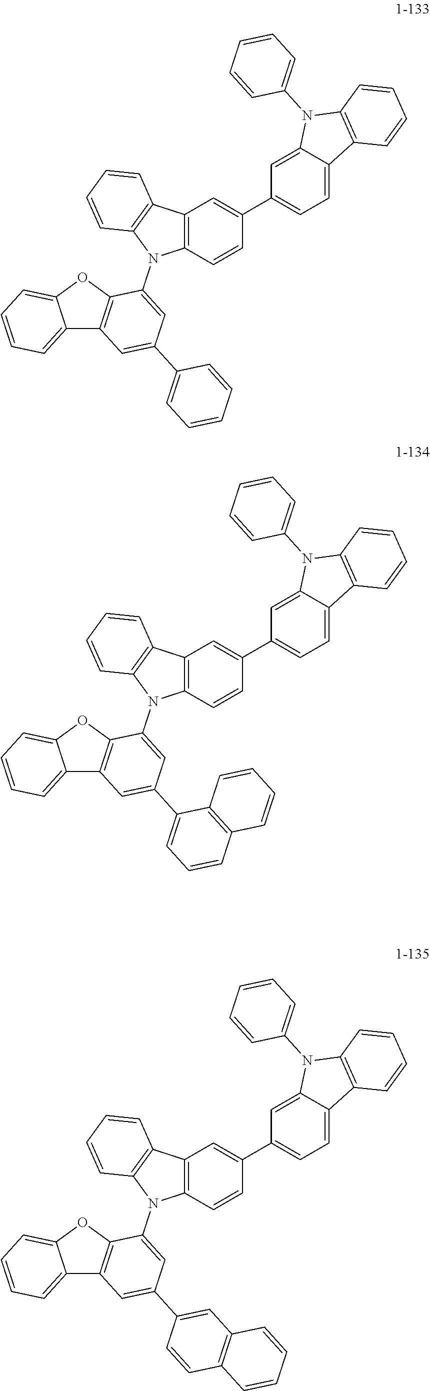 [SMILES: c1ccc(-c2cc(-n3c4ccccc4c4cc(-c5ccc6c7ccccc7n(-c7ccccc7)c6c5)ccc43)c3oc4ccccc4c3c2)cc1 ; c1ccc(-n2c3ccccc3c3ccc(-c4ccc5c(c4)c4ccccc4n5-c4cc(-c5cccc6ccccc56)cc5c4oc4ccccc45)cc32)cc1 ; c1ccc(-n2c3ccccc3c3ccc(-c4ccc5c(c4)c4ccccc4n5-c4cc(-c5ccc6ccccc6c5)cc5c4oc4ccccc45)cc32)cc1]

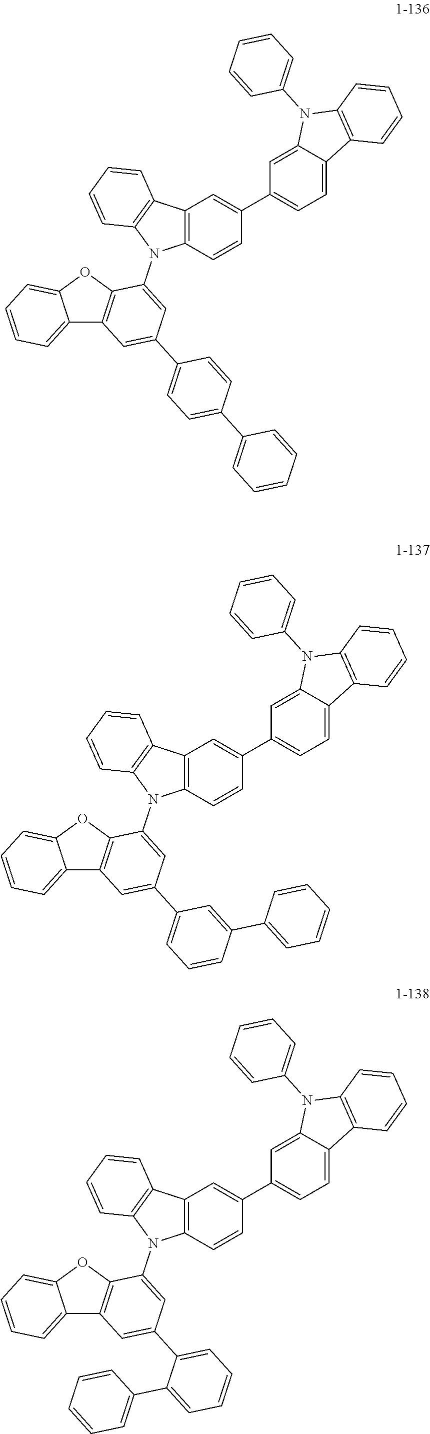 [SMILES: c1ccc(-c2ccc(-c3cc(-n4c5ccccc5c5cc(-c6ccc7c8ccccc8n(-c8ccccc8)c7c6)ccc54)c4oc5ccccc5c4c3)cc2)cc1 ; c1ccc(-c2cccc(-c3cc(-n4c5ccccc5c5cc(-c6ccc7c8ccccc8n(-c8ccccc8)c7c6)ccc54)c4oc5ccccc5c4c3)c2)cc1 ; c1ccc(-c2ccccc2-c2cc(-n3c4ccccc4c4cc(-c5ccc6c7ccccc7n(-c7ccccc7)c6c5)ccc43)c3oc4ccccc4c3c2)cc1]

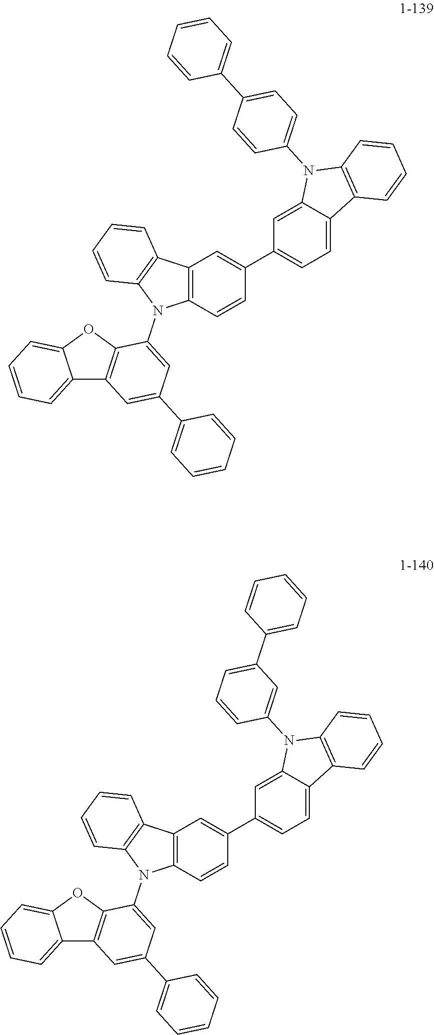 [SMILES: c1ccc(-c2ccc(-n3c4ccccc4c4ccc(-c5ccc6c(c5)c5ccccc5n6-c5cc(-c6ccccc6)cc6c5oc5ccccc56)cc43)cc2)cc1 ; c1ccc(-c2cccc(-n3c4ccccc4c4ccc(-c5ccc6c(c5)c5ccccc5n6-c5cc(-c6ccccc6)cc6c5oc5ccccc56)cc43)c2)cc1]

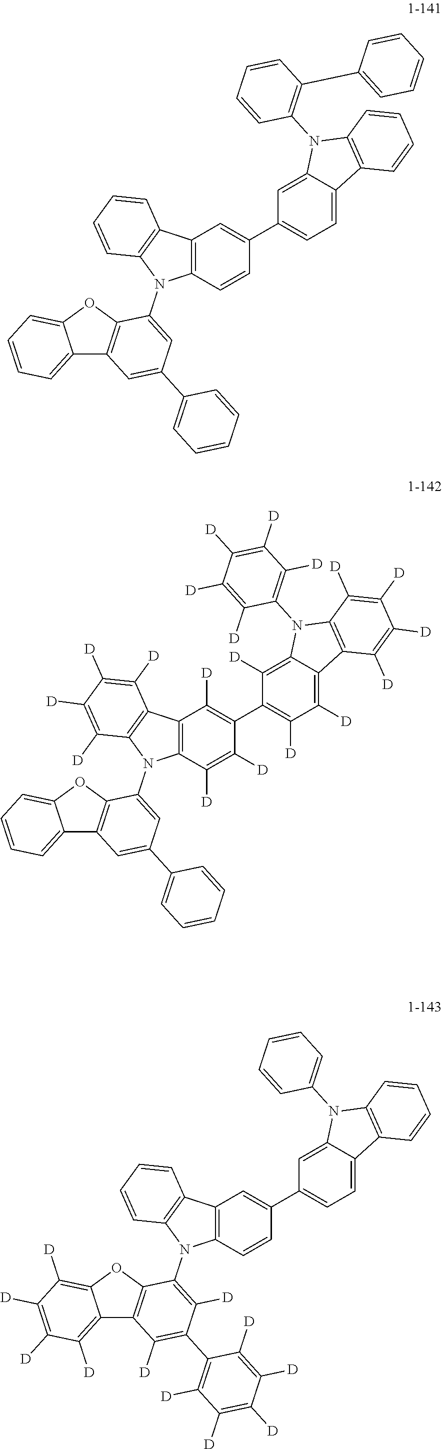 [SMILES: c1ccc(-c2cc(-n3c4ccccc4c4cc(-c5ccc6c7ccccc7n(-c7ccccc7-c7ccccc7)c6c5)ccc43)c3oc4ccccc4c3c2)cc1 ; [2H]c1c([2H])c([2H])c(-n2c3c([2H])c([2H])c([2H])c([2H])c3c3c([2H])c([2H])c(-c4c([2H])c([2H])c5c(c4[2H])c4c([2H])c([2H])c([2H])c([2H])c4n5-c4cc(-c5ccccc5)cc5c4oc4ccccc45)c([2H])c32)c([2H])c1[2H] ; [2H]c1c([2H])c([2H])c(-c2c([2H])c(-n3c4ccccc4c4cc(-c5ccc6c7ccccc7n(-c7ccccc7)c6c5)ccc43)c3oc4c([2H])c([2H])c([2H])c([2H])c4c3c2[2H])c([2H])c1[2H]]

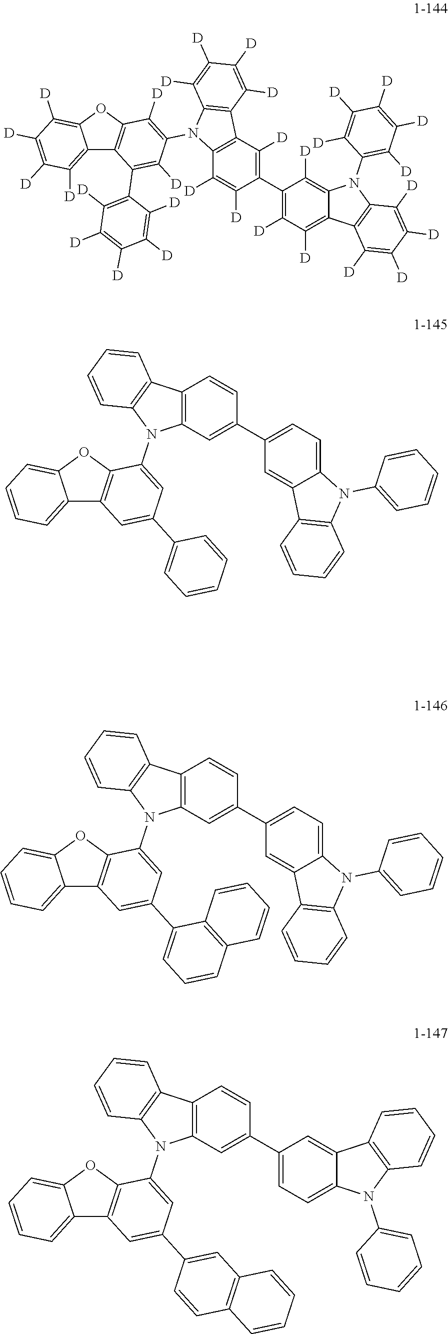

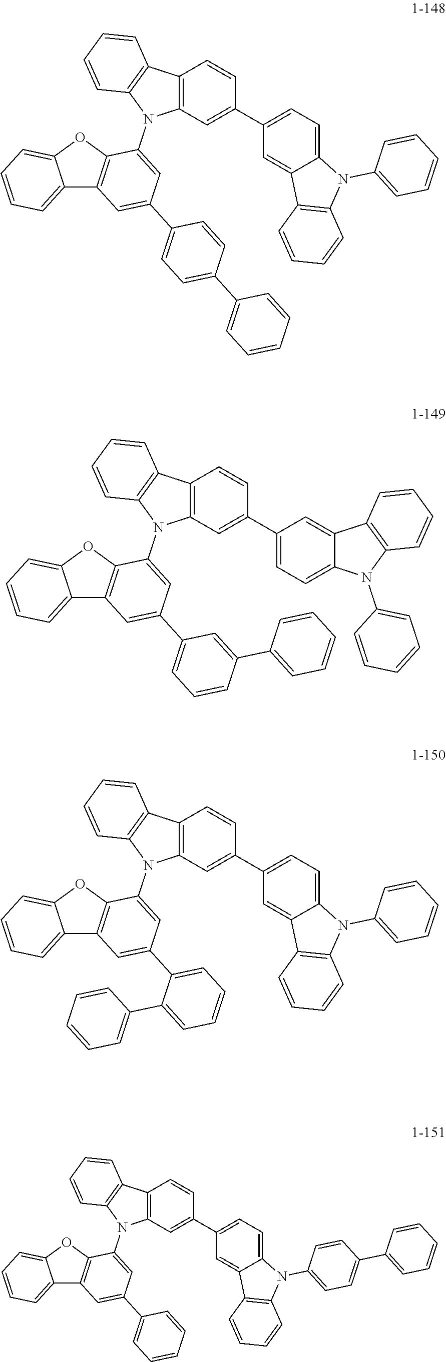 [SMILES: c1ccc(-c2ccc(-c3cc(-n4c5ccccc5c5ccc(-c6ccc7c(c6)c6ccccc6n7-c6ccccc6)cc54)c4oc5ccccc5c4c3)cc2)cc1 ; c1ccc(-c2cccc(-c3cc(-n4c5ccccc5c5ccc(-c6ccc7c(c6)c6ccccc6n7-c6ccccc6)cc54)c4oc5ccccc5c4c3)c2)cc1 ; c1ccc(-c2ccccc2-c2cc(-n3c4ccccc4c4ccc(-c5ccc6c(c5)c5ccccc5n6-c5ccccc5)cc43)c3oc4ccccc4c3c2)cc1 ; c1ccc(-c2ccc(-n3c4ccccc4c4cc(-c5ccc6c7ccccc7n(-c7cc(-c8ccccc8)cc8c7oc7ccccc78)c6c5)ccc43)cc2)cc1]

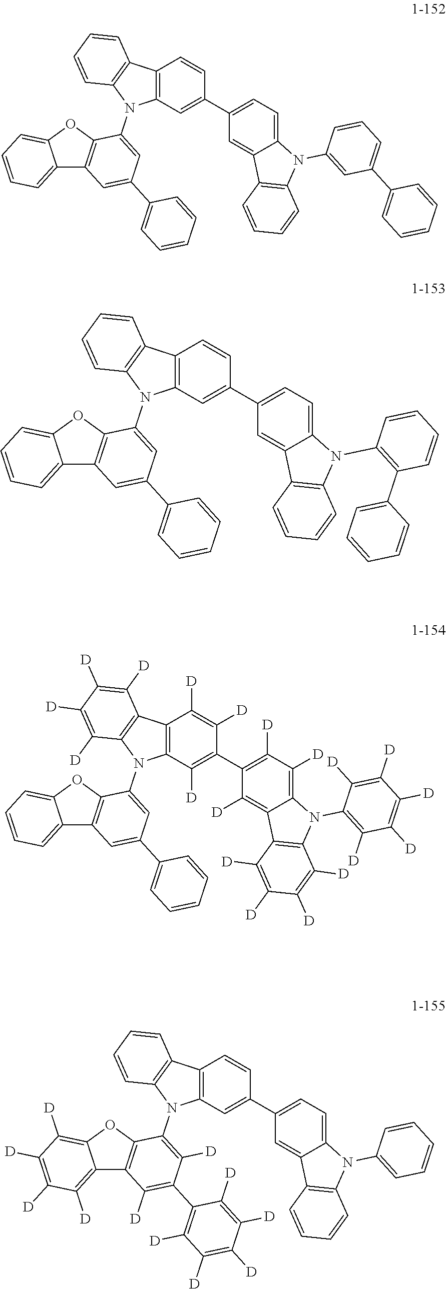

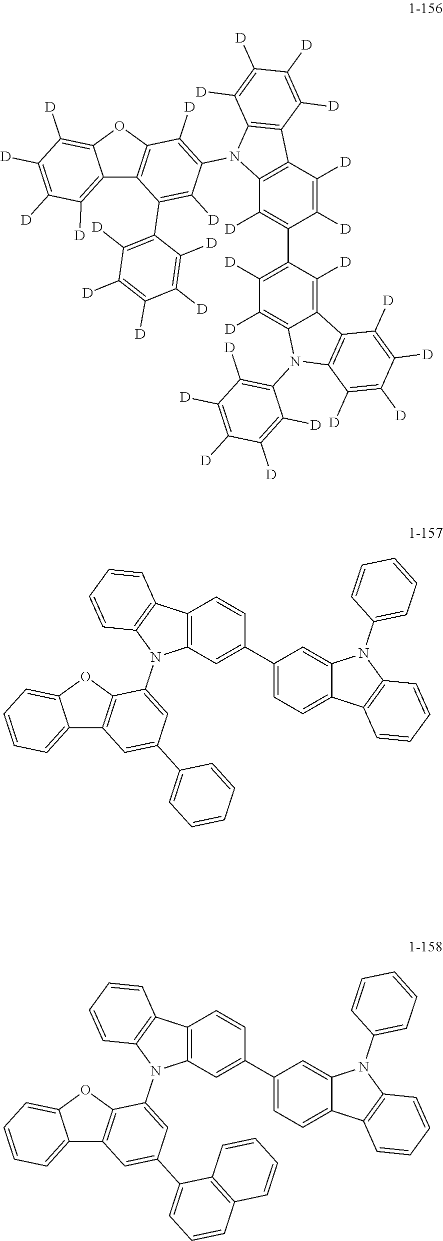 [SMILES: [2H]c1c([2H])c([2H])c(-c2c([2H])c(-n3c4c([2H])c([2H])c([2H])c([2H])c4c4c([2H])c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c43)c([2H])c3oc4c([2H])c([2H])c([2H])c([2H])c4c23)c([2H])c1[2H] ; c1ccc(-c2cc(-n3c4ccccc4c4ccc(-c5ccc6c7ccccc7n(-c7ccccc7)c6c5)cc43)c3oc4ccccc4c3c2)cc1 ; c1ccc(-n2c3ccccc3c3ccc(-c4ccc5c6ccccc6n(-c6cc(-c7cccc8ccccc78)cc7c6oc6ccccc67)c5c4)cc32)cc1]

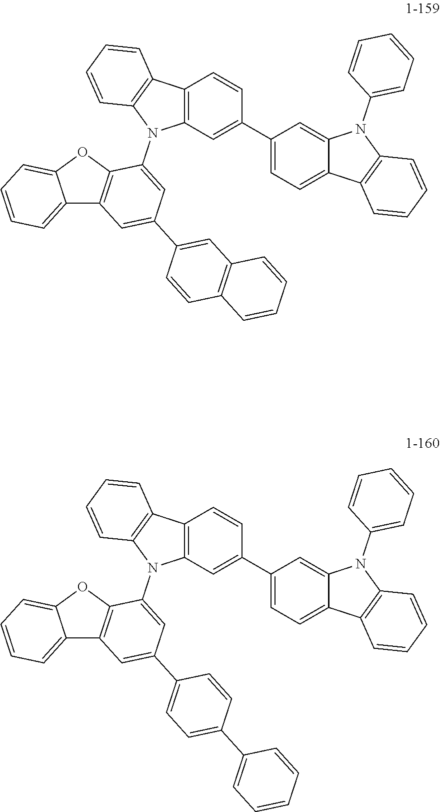 [SMILES: c1ccc(-n2c3ccccc3c3ccc(-c4ccc5c6ccccc6n(-c6cc(-c7ccc8ccccc8c7)cc7c6oc6ccccc67)c5c4)cc32)cc1 ; c1ccc(-c2ccc(-c3cc(-n4c5ccccc5c5ccc(-c6ccc7c8ccccc8n(-c8ccccc8)c7c6)cc54)c4oc5ccccc5c4c3)cc2)cc1]

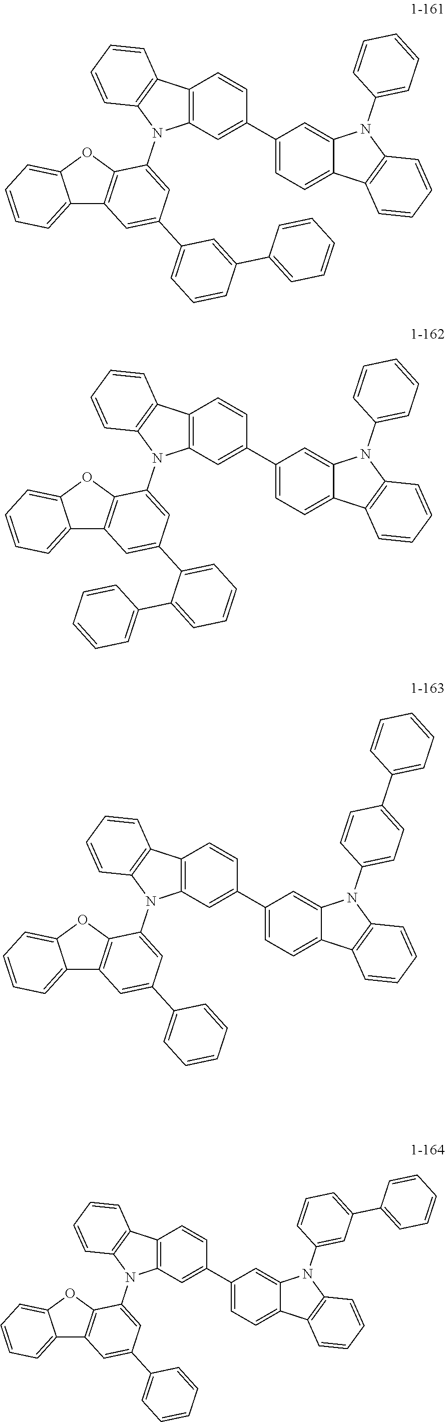 [SMILES: c1ccc(-c2cccc(-c3cc(-n4c5ccccc5c5ccc(-c6ccc7c8ccccc8n(-c8ccccc8)c7c6)cc54)c4oc5ccccc5c4c3)c2)cc1 ; c1ccc(-c2ccccc2-c2cc(-n3c4ccccc4c4ccc(-c5ccc6c7ccccc7n(-c7ccccc7)c6c5)cc43)c3oc4ccccc4c3c2)cc1 ; c1ccc(-c2ccc(-n3c4ccccc4c4ccc(-c5ccc6c7ccccc7n(-c7cc(-c8ccccc8)cc8c7oc7ccccc78)c6c5)cc43)cc2)cc1 ; c1ccc(-c2cccc(-n3c4ccccc4c4ccc(-c5ccc6c7ccccc7n(-c7cc(-c8ccccc8)cc8c7oc7ccccc78)c6c5)cc43)c2)cc1]

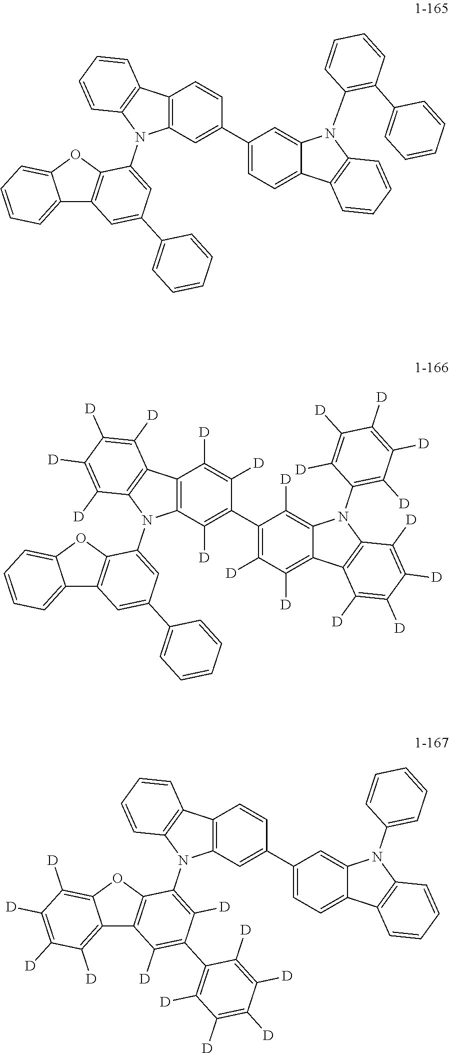 [SMILES: c1ccc(-c2cc(-n3c4ccccc4c4ccc(-c5ccc6c7ccccc7n(-c7ccccc7-c7ccccc7)c6c5)cc43)c3oc4ccccc4c3c2)cc1 ; [2H]c1c([2H])c([2H])c(-n2c3c([2H])c([2H])c([2H])c([2H])c3c3c([2H])c([2H])c(-c4c([2H])c([2H])c5c6c([2H])c([2H])c([2H])c([2H])c6n(-c6cc(-c7ccccc7)cc7c6oc6ccccc67)c5c4[2H])c([2H])c32)c([2H])c1[2H] ; [2H]c1c([2H])c([2H])c(-c2c([2H])c(-n3c4ccccc4c4ccc(-c5ccc6c7ccccc7n(-c7ccccc7)c6c5)cc43)c3oc4c([2H])c([2H])c([2H])c([2H])c4c3c2[2H])c([2H])c1[2H]]

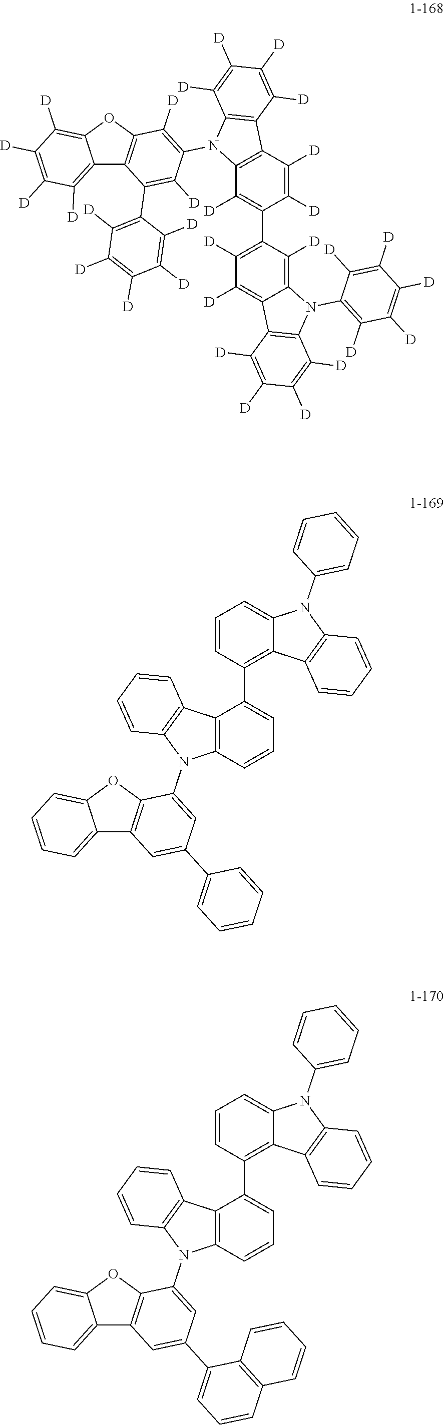 [SMILES: [2H]c1c([2H])c([2H])c(-c2c([2H])c(-n3c4c([2H])c([2H])c([2H])c([2H])c4c4c([2H])c([2H])c(-c5c([2H])c([2H])c6c7c([2H])c([2H])c([2H])c([2H])c7n(-c7c([2H])c([2H])c([2H])c([2H])c7[2H])c6c5[2H])c([2H])c43)c([2H])c3oc4c([2H])c([2H])c([2H])c([2H])c4c23)c([2H])c1[2H] ; c1ccc(-c2cc(-n3c4ccccc4c4c(-c5cccc6c5c5ccccc5n6-c5ccccc5)cccc43)c3oc4ccccc4c3c2)cc1 ; c1ccc(-n2c3ccccc3c3c(-c4cccc5c4c4ccccc4n5-c4cc(-c5cccc6ccccc56)cc5c4oc4ccccc45)cccc32)cc1]

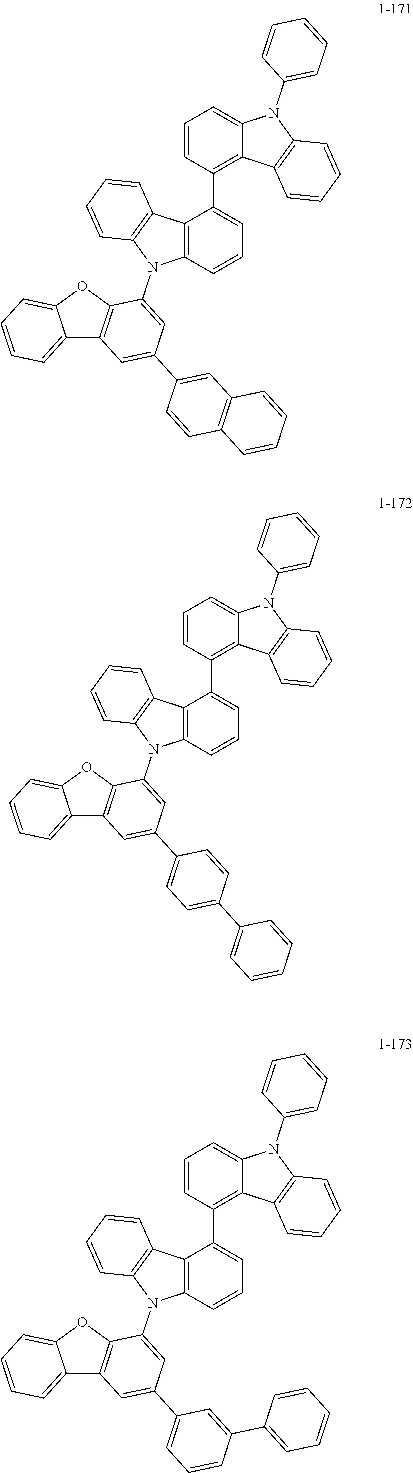 [SMILES: c1ccc(-n2c3ccccc3c3c(-c4cccc5c4c4ccccc4n5-c4cc(-c5ccc6ccccc6c5)cc5c4oc4ccccc45)cccc32)cc1 ; c1ccc(-c2ccc(-c3cc(-n4c5ccccc5c5c(-c6cccc7c6c6ccccc6n7-c6ccccc6)cccc54)c4oc5ccccc5c4c3)cc2)cc1 ; c1ccc(-c2cccc(-c3cc(-n4c5ccccc5c5c(-c6cccc7c6c6ccccc6n7-c6ccccc6)cccc54)c4oc5ccccc5c4c3)c2)cc1]

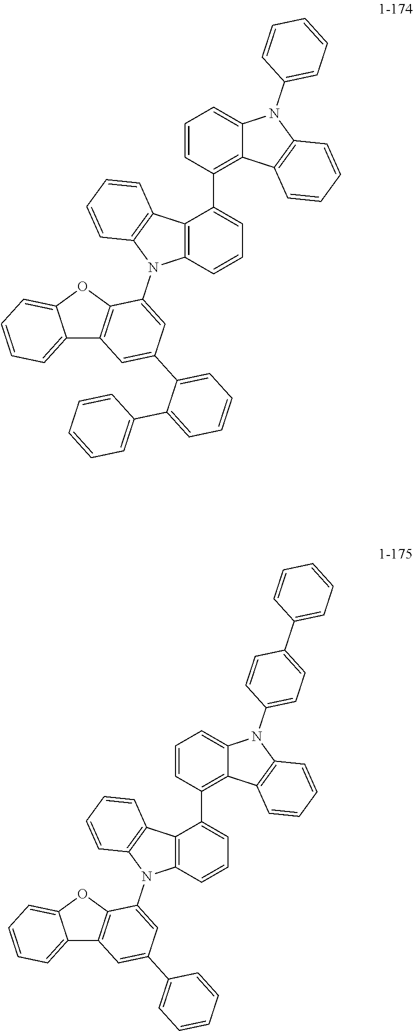 [SMILES: c1ccc(-c2ccccc2-c2cc(-n3c4ccccc4c4c(-c5cccc6c5c5ccccc5n6-c5ccccc5)cccc43)c3oc4ccccc4c3c2)cc1 ; c1ccc(-c2ccc(-n3c4ccccc4c4c(-c5cccc6c5c5ccccc5n6-c5cc(-c6ccccc6)cc6c5oc5ccccc56)cccc43)cc2)cc1]

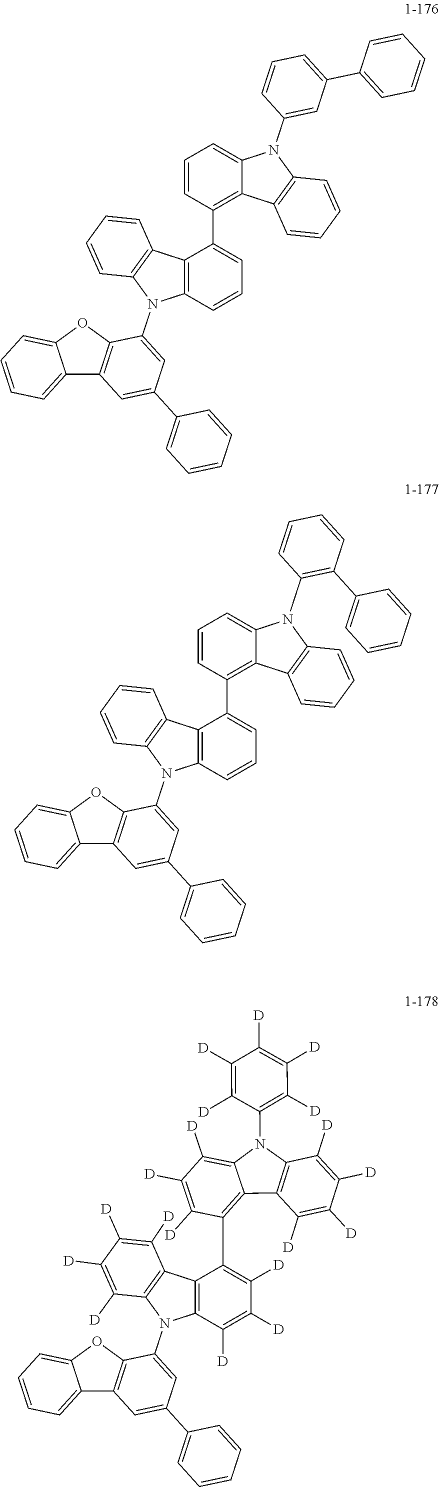 [SMILES: c1ccc(-c2cccc(-n3c4ccccc4c4c(-c5cccc6c5c5ccccc5n6-c5cc(-c6ccccc6)cc6c5oc5ccccc56)cccc43)c2)cc1 ; c1ccc(-c2cc(-n3c4ccccc4c4c(-c5cccc6c5c5ccccc5n6-c5ccccc5-c5ccccc5)cccc43)c3oc4ccccc4c3c2)cc1 ; [2H]c1c([2H])c([2H])c(-n2c3c([2H])c([2H])c([2H])c([2H])c3c3c(-c4c([2H])c([2H])c([2H])c5c4c4c([2H])c([2H])c([2H])c([2H])c4n5-c4cc(-c5ccccc5)cc5c4oc4ccccc45)c([2H])c([2H])c([2H])c32)c([2H])c1[2H]]

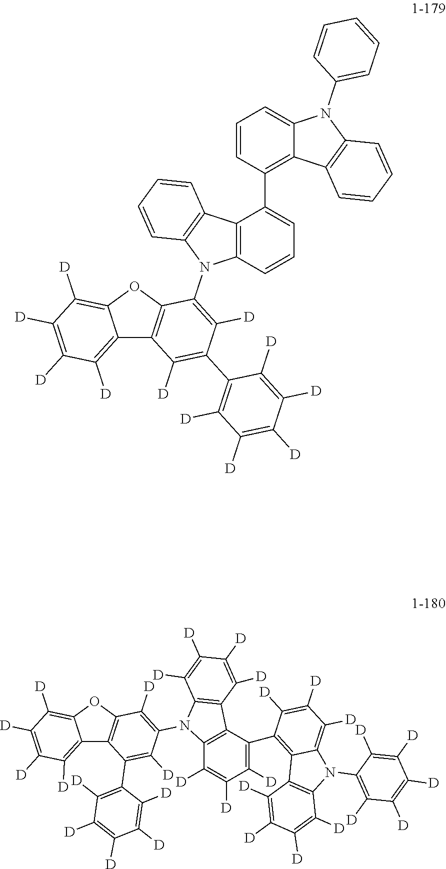 [SMILES: [2H]c1c([2H])c([2H])c(-c2c([2H])c(-n3c4ccccc4c4c(-c5cccc6c5c5ccccc5n6-c5ccccc5)cccc43)c3oc4c([2H])c([2H])c([2H])c([2H])c4c3c2[2H])c([2H])c1[2H] ; [2H]c1c([2H])c([2H])c(-c2c([2H])c(-n3c4c([2H])c([2H])c([2H])c([2H])c4c4c(-c5c([2H])c([2H])c([2H])c6c5c5c([2H])c([2H])c([2H])c([2H])c5n6-c5c([2H])c([2H])c([2H])c([2H])c5[2H])c([2H])c([2H])c([2H])c43)c([2H])c3oc4c([2H])c([2H])c([2H])c([2H])c4c23)c([2H])c1[2H]]

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a material for transporting holes, a light emitting layer material, an electron transport layer material, and a charge generation layer material, which are used for preparing an organic light emitting device, into the core structure.

In addition, it is possible to finely adjust an energy band gap by introducing various substituents into the structure of Chemical Formula 1, and meanwhile, it is possible to improve characteristics at the interface between organic materials and diversify the use of the material.

Meanwhile, the heterocyclic compound has a high glass transition temperature (Tg) and thus has excellent thermal stability. The increase in thermal stability becomes an important factor for providing a device with driving stability.

The heterocyclic compound according to an exemplary embodiment of the present application may be prepared by a multi-step chemical reaction. Some intermediate compounds are first prepared, and the compound of Chemical Formula 1 may be prepared from the intermediate compounds. More specifically, the heterocyclic compound according to an exemplary embodiment of the present application may be prepared based on the Preparation Examples to be described below.

Another exemplary embodiment of the present application provides an organic light emitting device comprising the heterocyclic compound represented by Chemical Formula 1. The ‘organic light emitting device” may be expressed by terms such as “organic light emitting diode”, “organic light emitting diodes (OLEDs)”, “OLED device”, and “organic electroluminescence device”.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

Specifically, the organic light emitting device according to an exemplary embodiment of the present application comprises a first electrode, a second electrode provided to face the first electrode and one or more organic material layers provided between the first electrode and the second electrode, in which one or more layers of the organic material layer comprise the heterocyclic compound represented by Chemical Formula 1. When the heterocyclic compound represented by Chemical Formula 1 is comprised in the organic material layer, the light emitting efficiency and service life of the organic light emitting device are excellent.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a green organic light emitting device, and the heterocyclic compound according to Chemical Formula 1 may be used as a material for the green organic light emitting device.

In an exemplary embodiment of the present application, the organic light emitting device may be a red organic light emitting device, and the heterocyclic compound according to Chemical Formula 1 may be used as a material for the red organic light emitting device.

In an exemplary embodiment of the present application, the organic light emitting device may be a blue organic light emitting device, and the heterocyclic compound according to Chemical Formula 1 may be used as a material for the blue organic light emitting device.

Further, the organic material layer comprises a hole transport layer, and the hole transport layer comprises the heterocyclic compound represented by Chemical Formula 1. When the heterocyclic compound represented by Chemical Formula 1 is comprised in the hole transport layer among the organic material layers, the light emitting efficiency and service life of the organic light emitting device are better.

In addition, the organic material layer comprises a light emitting layer, and the light emitting layer comprises the heterocyclic compound represented by Chemical Formula 1. When the heterocyclic compound represented by Chemical Formula 1 is comprised in the light emitting layer among the organic material layers, the light emitting efficiency and service life of the organic light emitting device are better.

An exemplary embodiment of the present application provides an organic light emitting device in which one or more layers of the organic material layer comprise both the heterocyclic compound represented by Chemical Formula 1 and a heterocyclic compound represented by the following Chemical Formula 2.

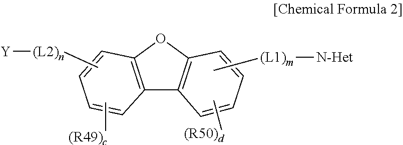

In Chemical Formula 2,
N-Het is a monocyclic or polycyclic heterocyclic group which is substituted or unsubstituted and comprises one or more N's,
L1 and L2 are the same as or different from each other, and are each independently a direct bond; a substituted or unsubstituted arylene group having 6 to 60 carbon atoms; or a substituted or unsubstituted heteroarylene group having 2 to 60 carbon atoms, m and n are each an integer from 0 to 3, when m is 2 or higher, L1's are the same as or different from each other, and when n is 2 or higher, L2's are the same as or different from each other,
R49 and R50 are the same as or different from each other, and are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or a substituted or unsubstituted hetero ring, c and d are each an integer from 0 to 3, when c is 2 or higher, R49's are the same as or different from each other, and when d is 2 or higher, R50's are the same as or different from each other, and
Y is a hole transporting group; or a substituted or unsubstituted aryl group having 6 to 60 carbon atoms.

In an exemplary embodiment of the present application, the N-Het, L1, L2, R49 and R50 are substituents substituted with one or more deuteriums, and the content of the substituted deuterium may be 0% to 100%.

In an exemplary embodiment of the present application, the N-Het is a monocyclic or polycyclic heterocyclic group comprising one or more N's substituted with one or more deuteriums, and the content of the substituted deuterium may be 0% to 100%.

In an exemplary embodiment of the present application, L1 and L2 are the same as or different from each other, and are each independently an arylene group having 6 to 60 carbon atoms, which is substituted with one or more deuteriums; or a heteroarylene group having 2 to 60 carbon atoms, which is substituted with one or more deuteriums, and the content of the substituted deuterium may be 0% to 100%.

In an exemplary embodiment of the present application, R49 and R50 are the same as or different from each other, and are each independently selected from the group consisting of an alkyl group having 1 to 60 carbon atoms, which is substituted with one or more deuteriums; an alkenyl group having 2 to 60 carbon atoms, which is substituted with one or more deuteriums; an alkynyl group having 2 to 60 carbon atoms, which is substituted with one or more deuteriums; an alkoxy group having 1 to 20 carbon atoms, which is substituted with one or more deuteriums; a cycloalkyl group having 3 to 60 carbon atoms, which is substituted with one or more deuteriums; a heterocycloalkyl group having 2 to 60 carbon atoms, which is substituted with one or more deuteriums; an aryl group having 6 to 60 carbon atoms, which is substituted with one or more deuteriums; a heteroaryl group having 2 to 60 carbon atoms, which is substituted with one or more deuteriums; a phosphine oxide group which is substituted with one or more deuteriums; and an amine group which is substituted with one or more deuteriums, two or more adjacent groups are bonded to each other to form a substituted or unsubstituted aliphatic or aromatic hydrocarbon ring or a substituted or unsubstituted hetero ring, and the content of the substituted deuterium may be 0% to 100%.

An exemplary embodiment of the present application provides an organic light emitting device comprising a first electrode, a second electrode and an organic material layer having one or more layers provided between the first electrode and the second electrode, in which one or more layers of the organic material layer comprise both the heterocyclic compound represented by Chemical Formula 1 and the heterocyclic compound represented by Chemical Formula 2.

In an exemplary embodiment of the present application, provided is an organic light emitting device, in which at least one of the compound represented by Chemical Formula 1 and the compound represented by Chemical Formula 2 has a deuterium content of more than 0% and 100% or less.

An exemplary embodiment of the present application provides an organic light emitting device in which one or more layers of the organic material layer comprise both the heterocyclic compound represented by Chemical Formula 1 and a heterocyclic compound represented by the following Chemical Formula 2, the compound represented by Chemical Formula 1 is a compound which comprises deuterium or a substituent substituted with deuterium, and the compound represented by Chemical Formula 2 may be a compound which does not comprise deuterium or a substituent substituted with deuterium. That is, the compound represented by Chemical Formula 1 is a compound which comprises deuterium or a substituent substituted with deuterium, and the compound represented by Chemical Formula 2 may have a deuterium content of 0%.

An exemplary embodiment of the present application provides an organic light emitting device in which one or more layers of the organic material layer comprise both the heterocyclic compound represented by Chemical Formula 1 and a heterocyclic compound represented by the following Chemical Formula 2, the compound represented by Chemical Formula 1 is a compound which does not comprise deuterium or a substituent substituted with deuterium, and the compound represented by Chemical Formula 2 may be a compound which comprises deuterium or a substituent substituted with deuterium, the compound represented by Chemical Formula 1 may be a compound having a deuterium content of 0%, and the compound represented by Chemical Formula 2 may be a compound which comprises deuterium or a substituent substituted with deuterium.

An exemplary embodiment of the present application provides an organic light emitting device in which one or more layers of the organic material layer comprise both the heterocyclic compound represented by Chemical Formula 1 and a heterocyclic compound represented by the following Chemical Formula 2, and both the compound represented by Chemical Formula 1 and the compound represented by Chemical Formula 2 may be a compound which comprises deuterium or a substituent substituted with deuterium.

In general, compounds bonded with hydrogen and compounds substituted with deuterium exhibit a difference in thermodynamic behavior. The reason for this is that the mass of a deuterium atom is 2-fold higher than that of hydrogen, but due to the difference in the mass of atoms, deuterium is characterized by having even lower vibration energy. In addition, the bond length of carbon and deuterium is shorter than that of hydrogen, and a dissociation energy used to break the bond is also stronger than that of hydrogen. This is because the van der Waals radius of deuterium is smaller than that of hydrogen, and thus the extension amplitude of a bond between carbon and deuterium becomes even narrower.

The deuterium-substituted compound is characterized in that the energy in the ground state is further lower than that of the hydrogen-substituted compound, and the shorter the bond length between carbon and deuterium is, the smaller the molecular hardcore volume is. Accordingly, the electrical polarizability may be reduced, the intermolecular interaction can be weakened, and the volume of the device thin film may be increased by weakening the intermolecular interaction. These characteristics induce an effect of lowering the crystallinity by creating the amorphous state of a thin film.

In conclusion, deuterium substitution may be effective in improving the heat resistance of an organic light emitting device, and accordingly, the service life and driving characteristics may be improved.

In the present specification, a hole transporting group means a functional group having a greater hole transporting property than an electron transporting property, and may be referred to as a P-type functional group.

In an exemplary embodiment of the present application, Chemical Formula 2 may be represented by one of the following Chemical Formulae 2-1 to 2-3.

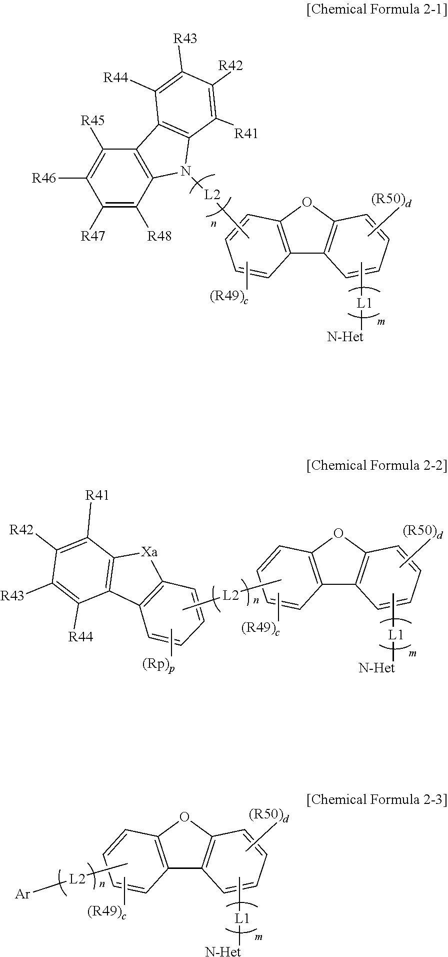

In Chemical Formulae 2-1 to 2-3,
the definitions of L1, L2, N-Het, R49, R50, m and n are the same as the definitions in Chemical Formula 2,
Xa is S, O, CReRf or NRg,
R41 to R48, Re, Rf, Rg and Rp are the same as or different from each other, and are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or a substituted or unsubstituted hetero ring, p is an integer from 0 to 3, when p is 2 or higher, Rp's are the same as or different from each other, and
Ar is a substituted or unsubstituted aryl group; or a substituted or unsubstituted silyl group.

In an exemplary embodiment of the present application, Xa is O or S.

In another exemplary embodiment, Xa is NRg, and Rg is an aryl group.

In still another exemplary embodiment, Xa is NRg, and Rg is a phenyl group.

In yet another exemplary embodiment, Xa is CReRf, and Re and Rf are an alkyl group.

In yet another exemplary embodiment, Xa is CReRf, and Re and Rf are a methyl group.

In an exemplary embodiment of the present application, Chemical Formula 2-1 may be represented by one of the following Chemical Formulae 2-1-1 to 2-1-4.

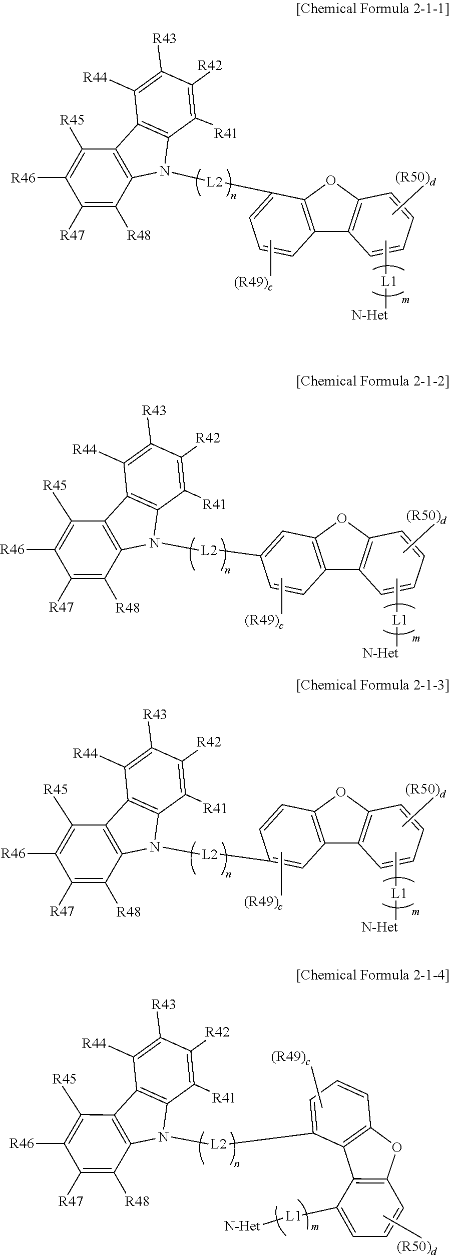

In Chemical Formulae 2-1-1 to 2-1-4, the definitions of R41 to R50, N-Het, L1, L2, c, d, m and n are the same as those in Chemical Formula 2-1.

In an exemplary embodiment of the present application, Chemical Formula 2-1 may be represented by one of the following Chemical Formulae 2-1-5 to 2-1-8.

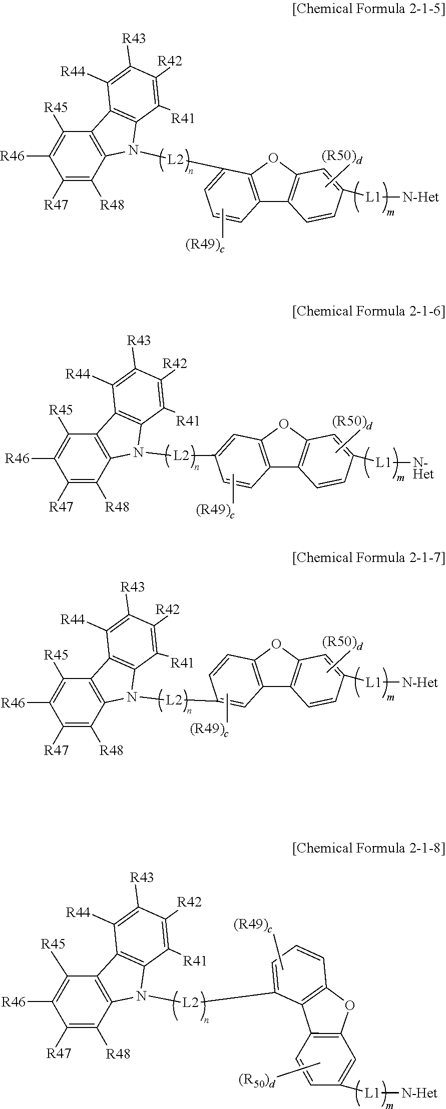

In Chemical Formulae 2-1-5 to 2-1-8, the definitions of R41 to R50, N-Het, L1, L2, c, d, m and n are the same as those in Chemical Formula 2-1.

In an exemplary embodiment of the present application, Chemical Formula 2-2 may be represented by one of the following Chemical Formulae 2-2-1 to 2-2-4.

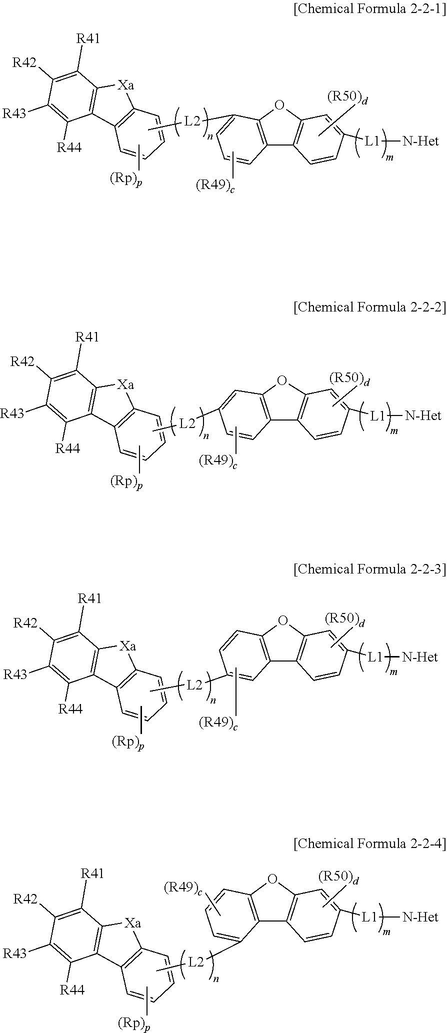

In Chemical Formulae 2-2-1 to 2-1-4, the definitions of R41 to R44, R49, R50, Rp, N-Het, L1, L2, c, d, m, n and p are the same as those in Chemical Formula 2-2.

In an exemplary embodiment of the present application, Chemical Formula 2-3 may be represented by the following 2-3-1 or 2-3-2.

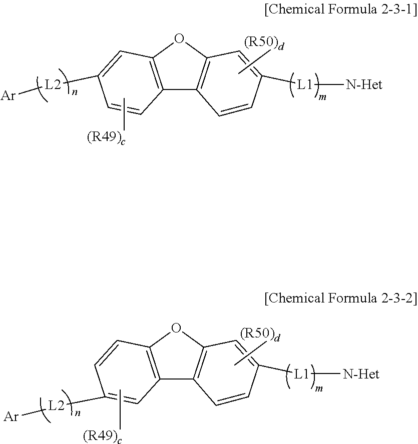

In Chemical Formulae 2-3-1 and 2-3-2, the definitions of Ar, R49, R50, Rp, N-Het, L1, L2, c, d, m and n are the same as those in Chemical Formula 2-3.

In an exemplary embodiment of the present application, the N-Het may be a heteroaryl group which is substituted or unsubstituted and a monocyclic heteroaryl group comprising one or more N's.

In an exemplary embodiment of the present application, the N-Het may be a bicyclic or more polycyclic heteroaryl group which is substituted or unsubstituted and comprises one or more N's.

In an exemplary embodiment of the present application, the N-Het may be a monocyclic or polycyclic heteroaryl group which is substituted or unsubstituted and comprises two or more N's.

In an exemplary embodiment of the present application, the N-Het may be a bicyclic or more polycyclic heteroaryl group comprising two or more N's.

In an exemplary embodiment of the present application, the N-Het of Chemical Formula 2 forms a carbon-carbon bond with L1.

In an exemplary embodiment of the present application, the N-Het is represented by one of the following Chemical Formulae 4 to 6.

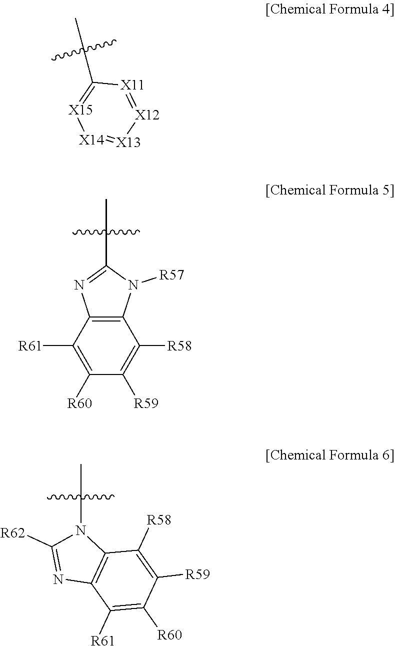

In Chemical Formulae 4 to 6,
X11 is CR51 or N, X12 is CR52 or N, X13 is CR53 or N,
X14 is CR54 or N, X15 is CR55 or N, and at least one of X11 to X15 is N,
R51 to R55 and R57 to R62 are the same as or different from each other, and are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or hetero ring, and

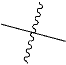

is a site which is linked to L or L1.

In an exemplary embodiment of the present application, R58 to R61 are the same as or different from each other, and are each independently hydrogen; deuterium; an aryl group; or a heteroaryl group.

In another exemplary embodiment, R58 to R61 are the same as or different from each other, and are each independently hydrogen; or deuterium.

In still another exemplary embodiment, R58 to R61 are hydrogen.

In an exemplary embodiment of the present application, R57 and R62 are the same as or different from each other, and are each independently hydrogen; deuterium; an aryl group; or a heteroaryl group.

In another exemplary embodiment, R57 and R62 are the same as or different from each other, and are each independently an aryl group; or a heteroaryl group.

In still another exemplary embodiment, R57 and R62 are the same as or different from each other, and are each independently an aryl group.

In yet another exemplary embodiment, R57 and R62 are a phenyl group.

In an exemplary embodiment of the present application, R51 to R55 are the same as or different from each other, and are each independently hydrogen; deuterium; an aryl group which is unsubstituted or substituted with an alkyl group; or a substituted or unsubstituted heteroaryl group.

In another exemplary embodiment, R51 to R55 are the same as or different from each other, and are each independently hydrogen; deuterium; an aryl group which is unsubstituted or substituted with an alkyl group; or a heteroaryl group.

In still another exemplary embodiment, R51 to R55 are the same as or different from each other, and are each independently hydrogen; an aryl group which is unsubstituted or substituted with a methyl group; or a heteroaryl group.

In yet another exemplary embodiment, R51 to R55 are the same as or different from each other, and are each independently hydrogen; a phenyl group; a biphenylyl group; a naphthyl group; a dimethylfluorenyl group; a dibenzofuran group; or a dibenzothiophene group.

In yet another exemplary embodiment, R52 and R54 are the same as or different from each other, and are each independently an aryl group which is unsubstituted or substituted with an alkyl group; or a heteroaryl group.

In yet another exemplary embodiment, R52 and R54 are the same as or different from each other, and are each independently a phenyl group; a biphenylyl group; a naphthyl group; a dimethylfluorenyl group; a dibenzofuran group; or a dibenzothiophene group.

In an exemplary embodiment of the present application, Chemical Formula 4 may be represented by one of the following Chemical Formulae 7 to 10. Here,

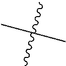

is a site which is linked to L or L1.

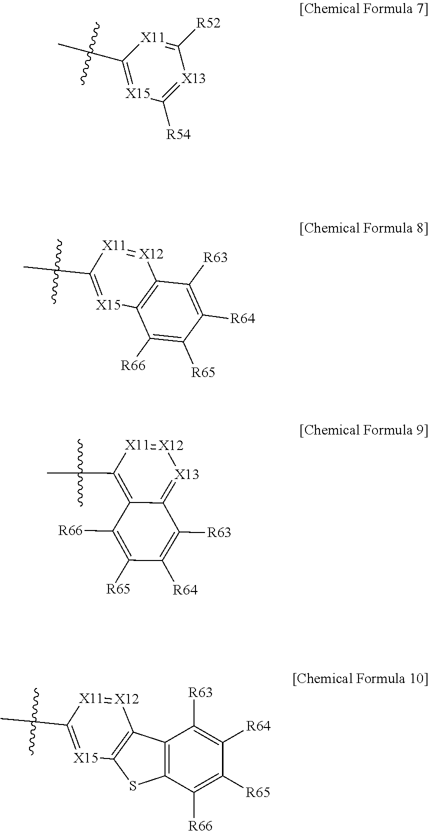

In Chemical Formula 7, one or more of X11, X13 and X15 are N, and the others are the same as those defined in Chemical Formula 4,
in Chemical Formula 8, one or more of X11, X12 and X15 are N, and the others are the same as those defined in Chemical Formula 4,
in Chemical Formula 9, one or more of X11 to X13 are N, and the others are the same as those defined in Chemical Formula 4,
in Chemical Formula 10, one or more of X11, X12 and X15 are N, and the others are the same as those defined in Chemical Formula 4, and
R52, R54 and R63 to R66 are the same as or different from each other, and are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or hetero ring.

In an exemplary embodiment of the present application, R63 to R66 are the same as or different from each other, and are each independently hydrogen; deuterium; an aryl group; or a heteroaryl group.

In another exemplary embodiment, R63 to R66 are the same as or different from each other, and are each independently hydrogen; deuterium; or an aryl group.

In still another exemplary embodiment, R63 to R66 are the same as or different from each other, and are each independently hydrogen; or an aryl group.

In yet another exemplary embodiment, R63 to R66 are the same as or different from each other, and are each independently hydrogen; a phenyl group; or a biphenylyl group.

In an exemplary embodiment of the present application, Chemical Formula 4 may be selected from the structural formulae of the following Group A.

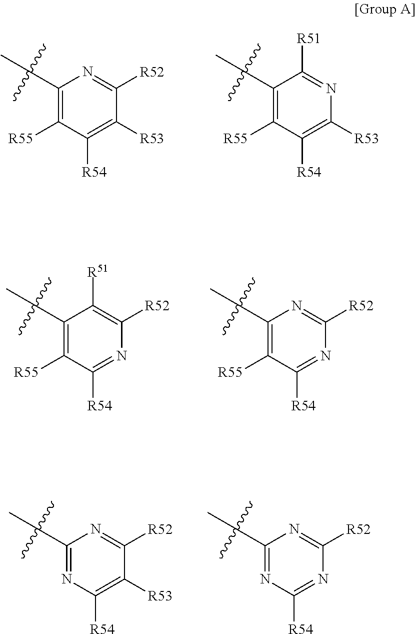

The definitions of R51 to R55 of the structural formulae of Group A are the same as those in Chemical

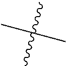

Formula 4, Here, is a site which is linked to L or L1.

In an exemplary embodiment of the present application, Chemical Formula 8 may be represented by the following Chemical Formula 11.

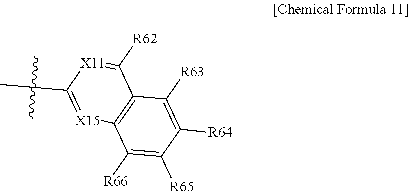

The definitions of X11, X15, and R62 to R66 of Chemical Formula 11 are the same as those defined in Formula 8, Here,

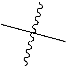

is a site which is linked to L or L1.

In an exemplary embodiment of the present application, Chemical Formula 9 may be represented by the following Chemical Formula 12.

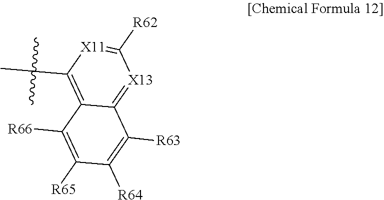

The definitions of X11, X13, and R62 to R66 of Chemical Formula 12 are the same as those defined in Formula 9. Here,

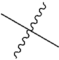

is a site which is linked to L or L1.

In an exemplary embodiment of the present application, Chemical Formula 8 may be represented by the following Chemical Formula 13.

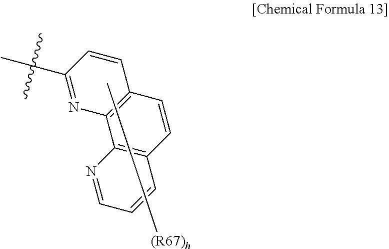

In Chemical Formula 13,
R67's are the same as or different from each other, and are selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or hetero ring, h is an integer from 0 to 7, when h is 2 or higher, R67's are the same as or different from each other. Here,

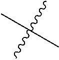

is a site which is linked to L or L1.

In an exemplary embodiment of the present specification, R67 is hydrogen; deuterium; an aryl group; or a heteroaryl group.

In another exemplary embodiment, R67 is hydrogen; deuterium; or an aryl group.

In still another exemplary embodiment, R67 is hydrogen; or an aryl group.

In yet another exemplary embodiment, R67 is hydrogen; or a phenyl group.

In an exemplary embodiment of the present application, Chemical Formula 10 may be represented by the following Chemical Formula 14.

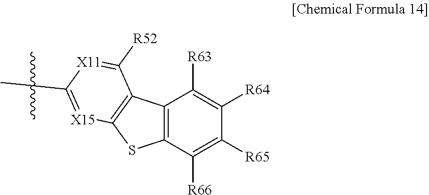

The definitions of X11, X15, R52 and R63 to R66 of Chemical Formula 14 are the same as those defined in Chemical Formula 10, Here,

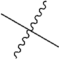

is a site which is linked to L or L1.

In another exemplary embodiment, L is a direct bond or an arylene group.

In still another exemplary embodiment, L is a direct bond or a phenylene group.

In yet another exemplary embodiment, R49 and R50 are hydrogen; or deuterium.

In yet another exemplary embodiment, R49 and R50 are hydrogen.

In yet another exemplary embodiment, R41 to R48 are hydrogen; deuterium; an aryl group which is unsubstituted or substituted with an alkyl group, an aryl group or a heteroaryl group; or a heteroaryl group which is unsubstituted or substituted with an aryl group or a heteroaryl group.

In yet another exemplary embodiment, R41 to R48 are hydrogen; deuterium; an aryl group; a heteroaryl group; or a heteroaryl group which is substituted with an aryl group.

In yet another exemplary embodiment, R41 to R48 are hydrogen; deuterium; a phenyl group; a dibenzofuran group; a dibenzothiophene group; a carbazole group; or a carbazole group which is substituted with phenyl.

In yet another exemplary embodiment, R41 to R48 are hydrogen; deuterium; a phenyl group; a dibenzofuran group; or a carbazole group which is substituted with phenyl.

In yet another exemplary embodiment, two adjacent substituents of R41 to R48 are bonded to each other to form a substituted or unsubstituted ring.

In yet another exemplary embodiment, two adjacent substituents of R41 to R48 are bonded to each other to form a ring which is unsubstituted or substituted with an aryl group or alkyl group.

In yet another exemplary embodiment, two adjacent substituents of R41 to R48 are bonded to each other to form an aromatic hydrocarbon ring or hetero ring, which is unsubstituted or substituted with an aryl group or alkyl group.

In yet another exemplary embodiment, two adjacent substituents of R41 to R48 are bonded to each other to form an aromatic hydrocarbon ring or hetero ring, which is unsubstituted or substituted with a phenyl group or methyl group.

In yet another exemplary embodiment, two adjacent substituents of R41 to R48 may be bonded to each other to form a benzene ring; an indole ring which is unsubstituted or substituted with a phenyl group; a benzothiophene ring; a benzofuran ring; or an indene ring which is unsubstituted or substituted with a methyl group.

In yet another exemplary embodiment, when two adjacent substituents of R41 to R48 of

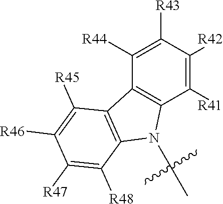

are bonded to each other to form a substituted or unsubstituted ring, the ring may be represented by the following Chemical Formula 15. Here,

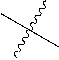

is a site which is linked to L2.

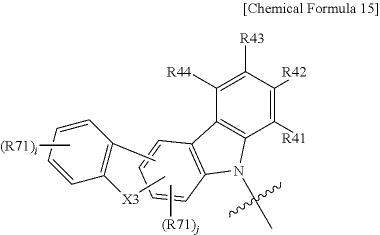

In Chemical Formula 15,
the definitions of R41 to R44 are the same as those defined in Chemical Formula 2-1,
X3 is O, S, CReRf or NRg,
Re, Rf, Rg, R71 and R72 are the same as or different from each other, and are selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or a substituted or unsubstituted hetero ring, i is an integer from 0 to 4, when i is 2 or higher, R71's are the same as or different from each other, and j is an integer from 0 to 2, and when j is 2 or higher, R72's are the same as or different from each other. Here,

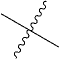

is a site which is linked to L2.

In yet another exemplary embodiment, Chemical Formula 15 may be selected from the structural formulae of the following Group B. Here,

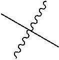

is a site which is linked to L2.

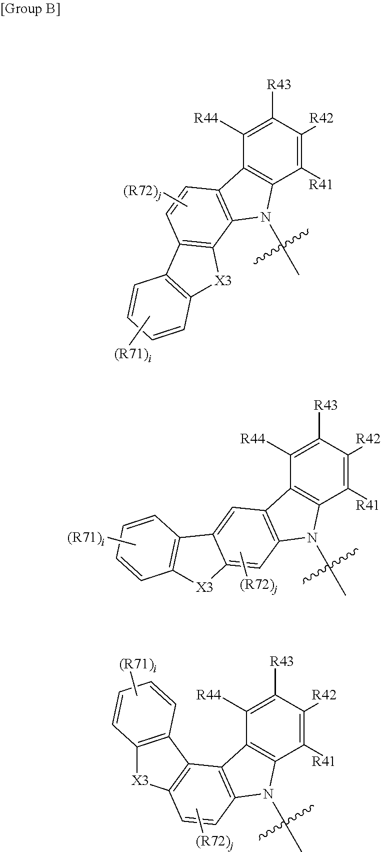

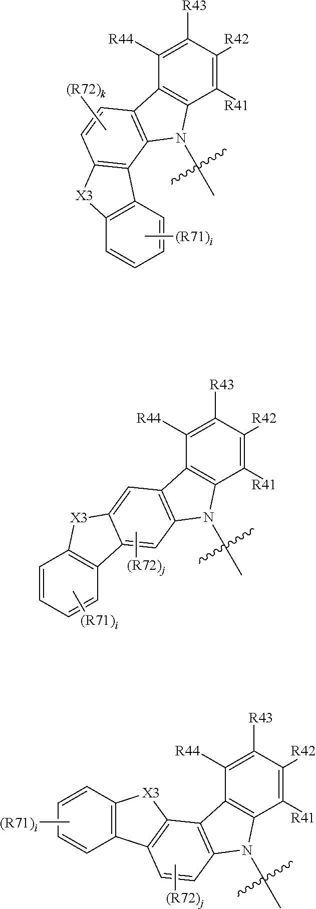

In the structural formulae,
the definitions of R41 to R44, X3, R71, R72, i and j are the same as those defined in Chemical Formula 15.

In an exemplary embodiment of the present application, X3 is O or S.

In another exemplary embodiment, X3 is NRg, and Rg is an aryl group.

In still another exemplary embodiment, X3 is NRg, and Rg is a phenyl group.

In yet another exemplary embodiment, X3 is CReRf, and Re and Rf are an alkyl group.

In yet another exemplary embodiment, X3 is CReRf, and Re and Rf are a methyl group.

In an exemplary embodiment of the present application, R71 is hydrogen; deuterium; an aryl group; or a heteroaryl group.

In another exemplary embodiment, R71 is hydrogen; deuterium; or an aryl group.

In still another exemplary embodiment, R71 is hydrogen; or a phenyl group.

In an exemplary embodiment of the present application, R72 is hydrogen; or deuterium.

In another exemplary embodiment, R72 is hydrogen.

In still another exemplary embodiment,

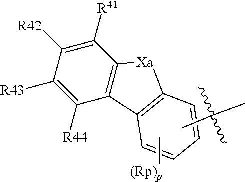

may be represented by the following Chemical Formula 16, when two adjacent substituents of R41 to R44 are bonded to each other to form a substituted or unsubstituted ring. Here,

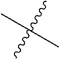

is a site which is linked to L2.

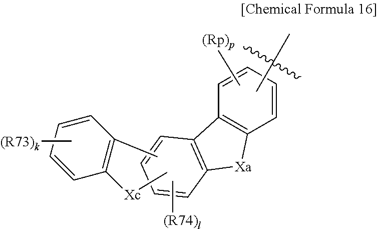

In Chemical Formula 16,
the definitions of Xa, Rp and p are the same as those defined in Chemical Formula 2-2,
Xc is O, S, CReRf or NRg,
Re, Rf, Rg, R73 and R74 are the same as or different from each other, and are selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or a substituted or unsubstituted hetero ring, k is an integer from 0 to 4, when k is 2 or higher, R73's are the same as or different from each other, and l is an integer from 0 to 2, and when 1 is 2 or higher, R74's are the same as or different from each other. Here,

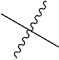

is a site which is linked to L2.

In yet another exemplary embodiment, Chemical Formula 13 may be selected from the following structural formulae.

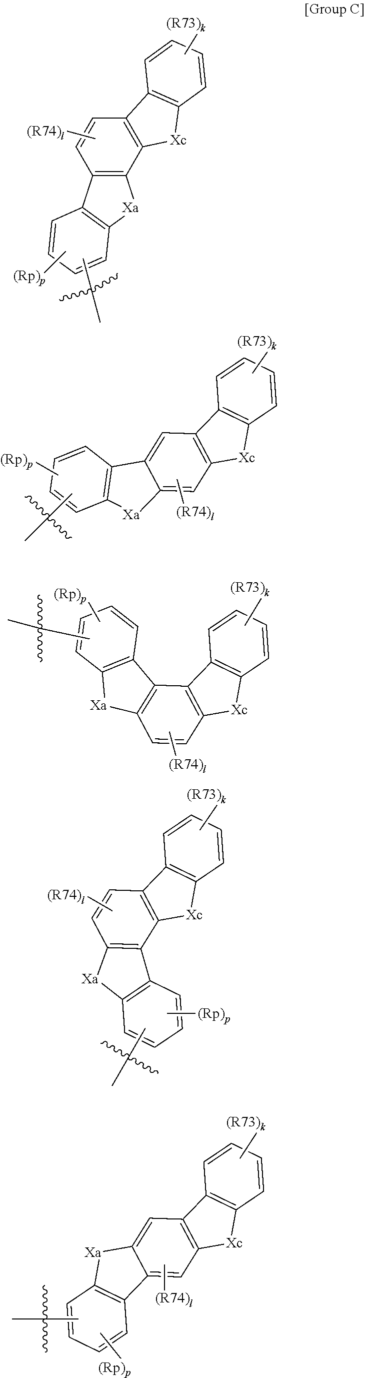

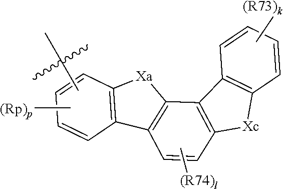

In the structural formulae,
the definitions of Xa, Rp, p, Xc, R73, R74, k and l are the same as those defined in Chemical Formula 13.

In an exemplary embodiment of the present application, Xa is O or S.

In another exemplary embodiment, Xa is NRg, and Rg is an aryl group.

In still another exemplary embodiment, Xa is NRg, and Rg is a phenyl group.

In yet another exemplary embodiment, Xa is CReRf, and Re and Rf are an alkyl group.

In yet another exemplary embodiment, Xa is CReRf, and Re and Rf are a methyl group.

In an exemplary embodiment of the present application, Xc is O or S.

In another exemplary embodiment, Xc is NRg, and Rg is an aryl group.

In still another exemplary embodiment, Xc is NRg, and Rg is a phenyl group.

In yet another exemplary embodiment, Xc is CReRf, and Re and Rf are an alkyl group.

In yet another exemplary embodiment, Xc is CReRf, and Re and Rf are a methyl group.

In yet another exemplary embodiment, R73 is hydrogen; deuterium; an aryl group; or a heteroaryl group.

In yet another exemplary embodiment, R73 is hydrogen; deuterium; or an aryl group.

In yet another exemplary embodiment, R73 is hydrogen; or a phenyl group.

In an exemplary embodiment of the present application, R74 is hydrogen; or deuterium.

In another exemplary embodiment, R74 is hydrogen.

In still another exemplary embodiment, L1 and L2 are the same as or different from each other, and are each independently a direct bond; an arylene group; or a heteroarylene group.

In yet another exemplary embodiment, L1 and L2 are the same as or different from each other, and are each independently a direct bond; a phenylene group; a naphthalene group; a biphenylylene group; or a divalent pyridine group.

According to an exemplary embodiment of the present application, Chemical Formula 2 may be represented by any one of the following compounds, but is not limited thereto.

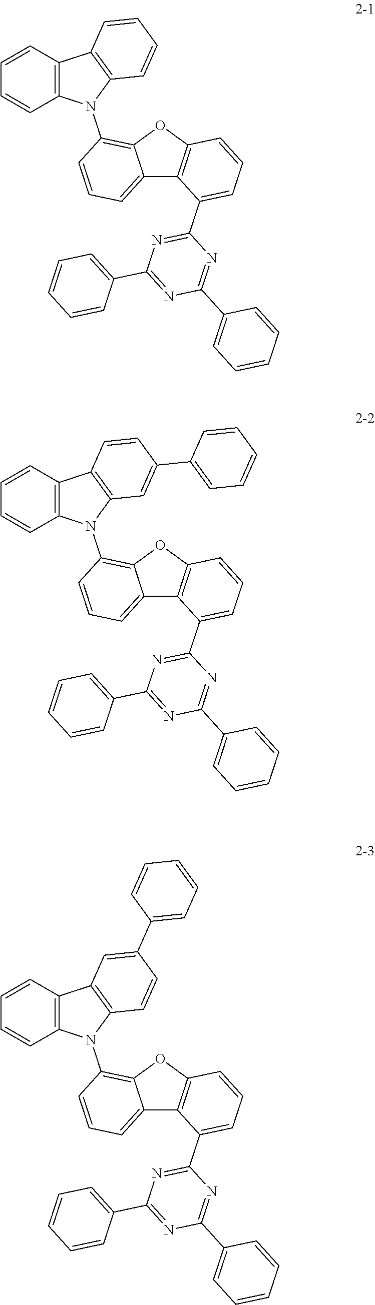 [SMILES: c1ccc(-c2nc(-c3ccccc3)nc(-c3cccc4oc5c(-n6c7ccccc7c7ccccc76)cccc5c34)n2)cc1 ; c1ccc(-c2ccc3c4ccccc4n(-c4cccc5c4oc4cccc(-c6nc(-c7ccccc7)nc(-c7ccccc7)n6)c45)c3c2)cc1 ; c1ccc(-c2ccc3c(c2)c2ccccc2n3-c2cccc3c2oc2cccc(-c4nc(-c5ccccc5)nc(-c5ccccc5)n4)c23)cc1]

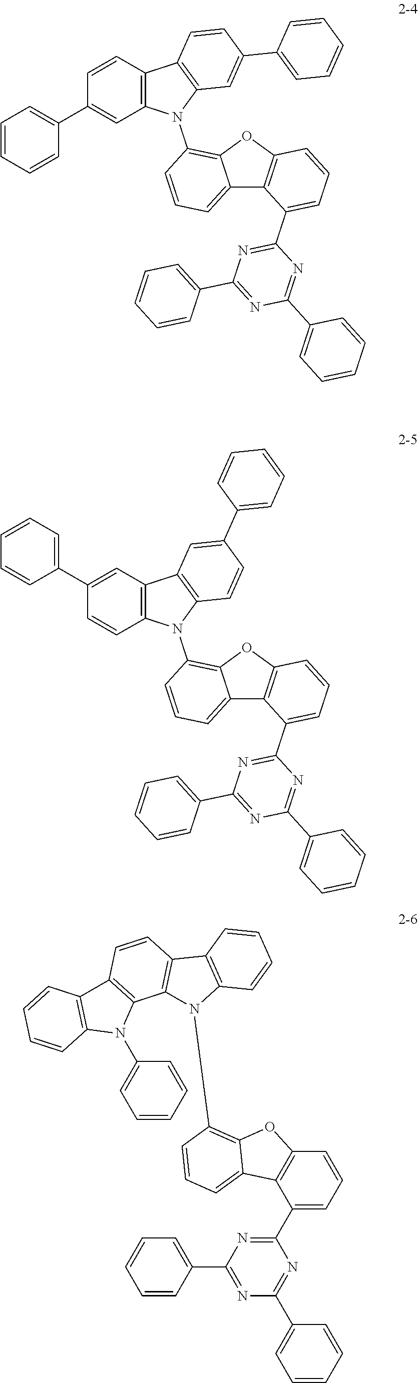 [SMILES: c1ccc(-c2ccc3c4ccc(-c5ccccc5)cc4n(-c4cccc5c4oc4cccc(-c6nc(-c7ccccc7)nc(-c7ccccc7)n6)c45)c3c2)cc1 ; c1ccc(-c2ccc3c(c2)c2cc(-c4ccccc4)ccc2n3-c2cccc3c2oc2cccc(-c4nc(-c5ccccc5)nc(-c5ccccc5)n4)c23)cc1 ; c1ccc(-c2nc(-c3ccccc3)nc(-c3cccc4oc5c(-n6c7ccccc7c7ccc8c9ccccc9n(-c9ccccc9)c8c76)cccc5c34)n2)cc1]

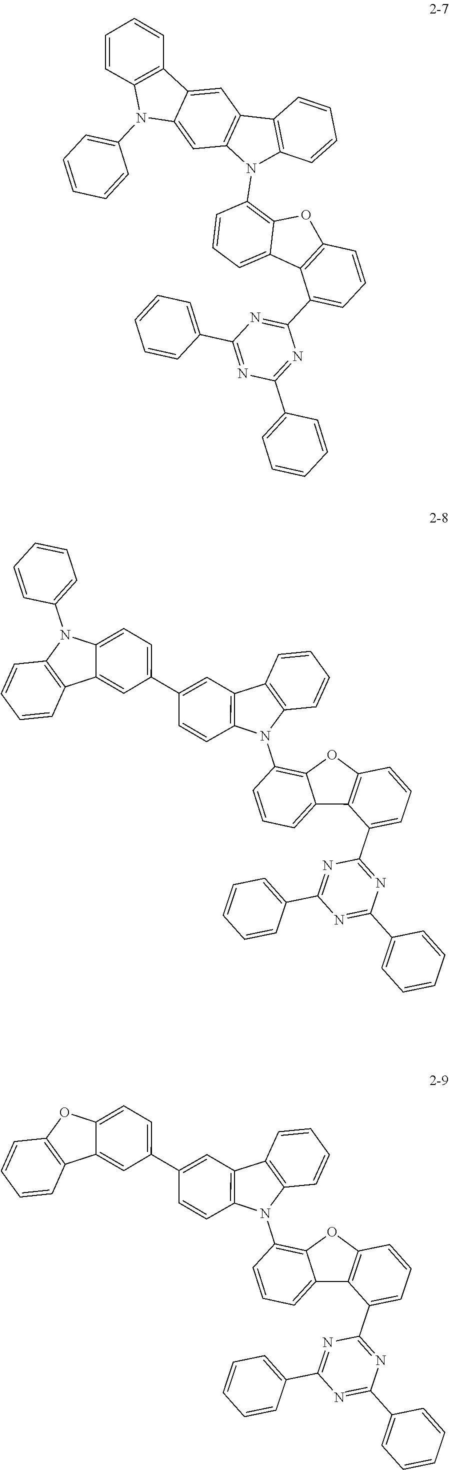 [SMILES: c1ccc(-c2nc(-c3ccccc3)nc(-c3cccc4oc5c(-n6c7ccccc7c7cc8c9ccccc9n(-c9ccccc9)c8cc76)cccc5c34)n2)cc1 ; c1ccc(-c2nc(-c3ccccc3)nc(-c3cccc4oc5c(-n6c7ccccc7c7cc(-c8ccc9c(c8)c8ccccc8n9-c8ccccc8)ccc76)cccc5c34)n2)cc1 ; c1ccc(-c2nc(-c3ccccc3)nc(-c3cccc4oc5c(-n6c7ccccc7c7cc(-c8ccc9oc%10ccccc%10c9c8)ccc76)cccc5c34)n2)cc1]

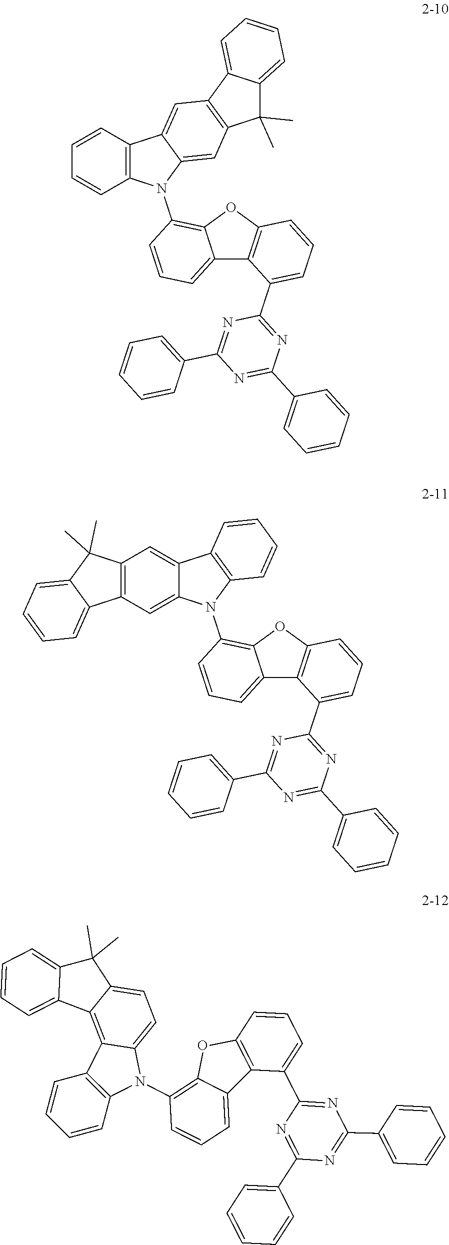 [SMILES: CC1(C)c2ccccc2-c2cc3c4ccccc4n(-c4cccc5c4oc4cccc(-c6nc(-c7ccccc7)nc(-c7ccccc7)n6)c45)c3cc21 ; CC1(C)c2ccccc2-c2cc3c(cc21)c1ccccc1n3-c1cccc2c1oc1cccc(-c3nc(-c4ccccc4)nc(-c4ccccc4)n3)c12 ; CC1(C)c2ccccc2-c2c1ccc1c2c2ccccc2n1-c1cccc2c1oc1cccc(-c3nc(-c4ccccc4)nc(-c4ccccc4)n3)c12]

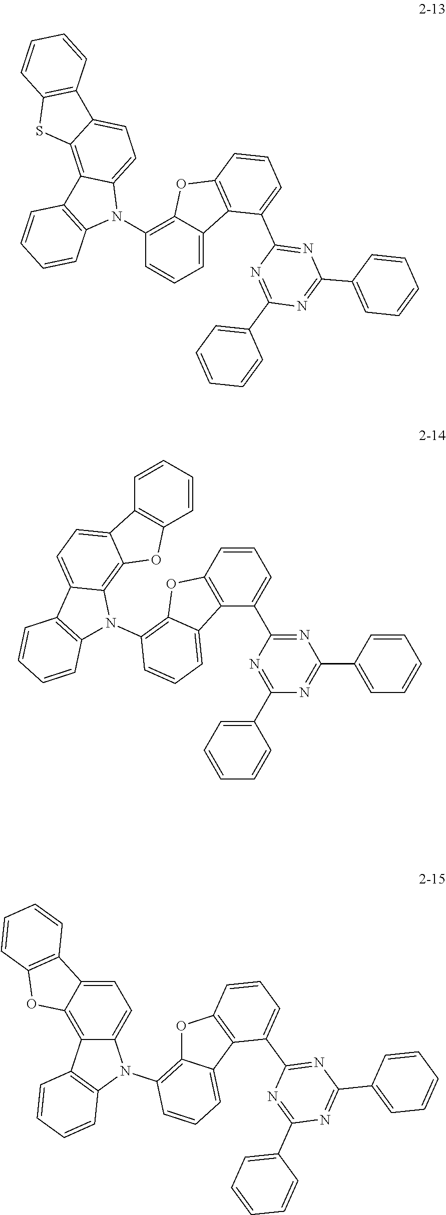 [SMILES: c1ccc(-c2nc(-c3ccccc3)nc(-c3cccc4oc5c(-n6c7ccccc7c7c8sc9ccccc9c8ccc76)cccc5c34)n2)cc1 ; c1ccc(-c2nc(-c3ccccc3)nc(-c3cccc4oc5c(-n6c7ccccc7c7ccc8c9ccccc9oc8c76)cccc5c34)n2)cc1 ; c1ccc(-c2nc(-c3ccccc3)nc(-c3cccc4oc5c(-n6c7ccccc7c7c8oc9ccccc9c8ccc76)cccc5c34)n2)cc1]

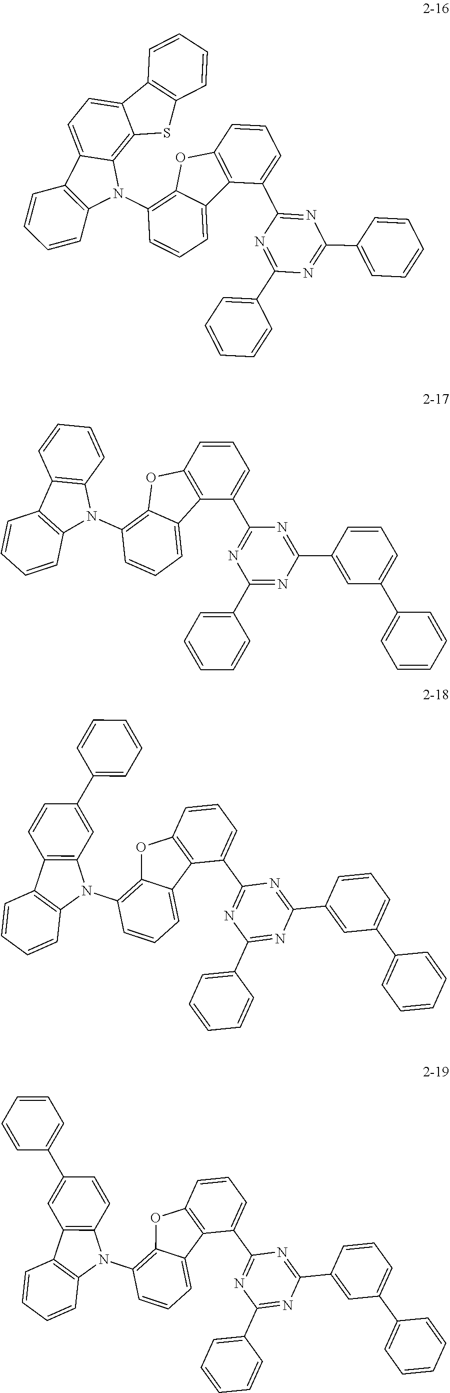 [SMILES: c1ccc(-c2nc(-c3ccccc3)nc(-c3cccc4oc5c(-n6c7ccccc7c7ccc8c9ccccc9sc8c76)cccc5c34)n2)cc1 ; c1ccc(-c2cccc(-c3nc(-c4ccccc4)nc(-c4cccc5oc6c(-n7c8ccccc8c8ccccc87)cccc6c45)n3)c2)cc1 ; c1ccc(-c2cccc(-c3nc(-c4ccccc4)nc(-c4cccc5oc6c(-n7c8ccccc8c8ccc(-c9ccccc9)cc87)cccc6c45)n3)c2)cc1 ; c1ccc(-c2cccc(-c3nc(-c4ccccc4)nc(-c4cccc5oc6c(-n7c8ccccc8c8cc(-c9ccccc9)ccc87)cccc6c45)n3)c2)cc1]

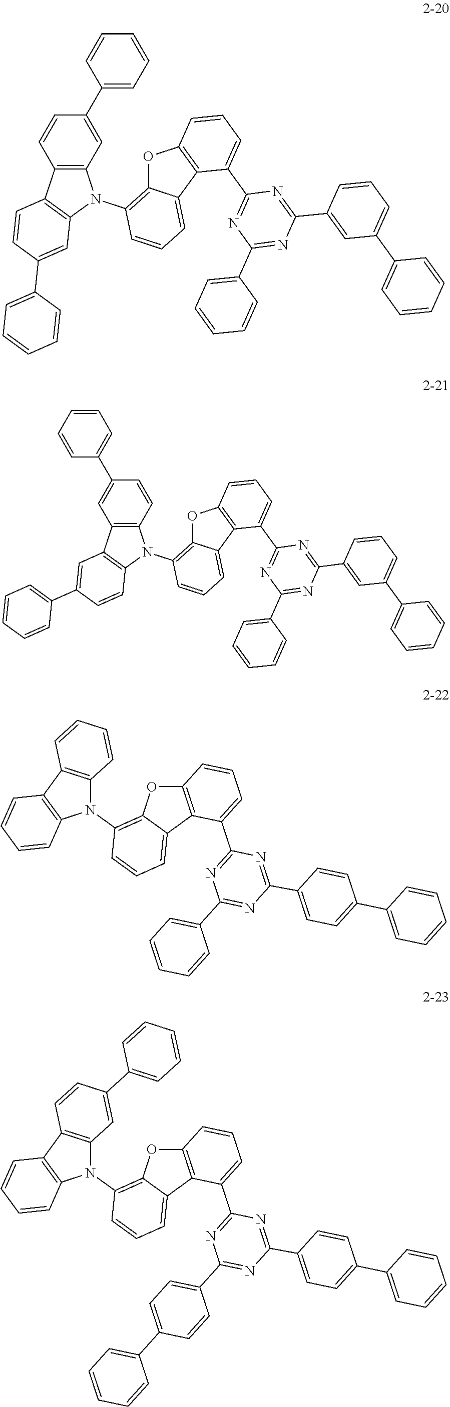 [SMILES: c1ccc(-c2cccc(-c3nc(-c4ccccc4)nc(-c4cccc5oc6c(-n7c8cc(-c9ccccc9)ccc8c8ccc(-c9ccccc9)cc87)cccc6c45)n3)c2)cc1 ; c1ccc(-c2cccc(-c3nc(-c4ccccc4)nc(-c4cccc5oc6c(-n7c8ccc(-c9ccccc9)cc8c8cc(-c9ccccc9)ccc87)cccc6c45)n3)c2)cc1 ; c1ccc(-c2ccc(-c3nc(-c4ccccc4)nc(-c4cccc5oc6c(-n7c8ccccc8c8ccccc87)cccc6c45)n3)cc2)cc1 ; c1ccc(-c2ccc(-c3nc(-c4ccc(-c5ccccc5)cc4)nc(-c4cccc5oc6c(-n7c8ccccc8c8ccc(-c9ccccc9)cc87)cccc6c45)n3)cc2)cc1]

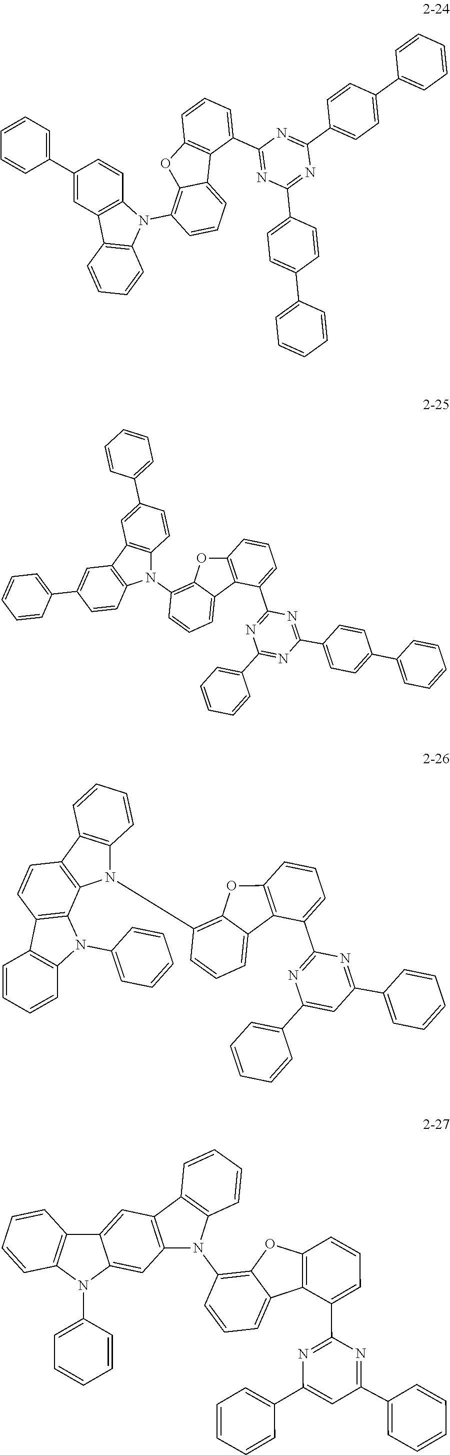 [SMILES: c1ccc(-c2ccc(-c3nc(-c4ccc(-c5ccccc5)cc4)nc(-c4cccc5oc6c(-n7c8ccccc8c8cc(-c9ccccc9)ccc87)cccc6c45)n3)cc2)cc1 ; c1ccc(-c2ccc(-c3nc(-c4ccccc4)nc(-c4cccc5oc6c(-n7c8ccc(-c9ccccc9)cc8c8cc(-c9ccccc9)ccc87)cccc6c45)n3)cc2)cc1 ; c1ccc(-c2cc(-c3ccccc3)nc(-c3cccc4oc5c(-n6c7ccccc7c7ccc8c9ccccc9n(-c9ccccc9)c8c76)cccc5c34)n2)cc1 ; c1ccc(-c2cc(-c3ccccc3)nc(-c3cccc4oc5c(-n6c7ccccc7c7cc8c9ccccc9n(-c9ccccc9)c8cc76)cccc5c34)n2)cc1]

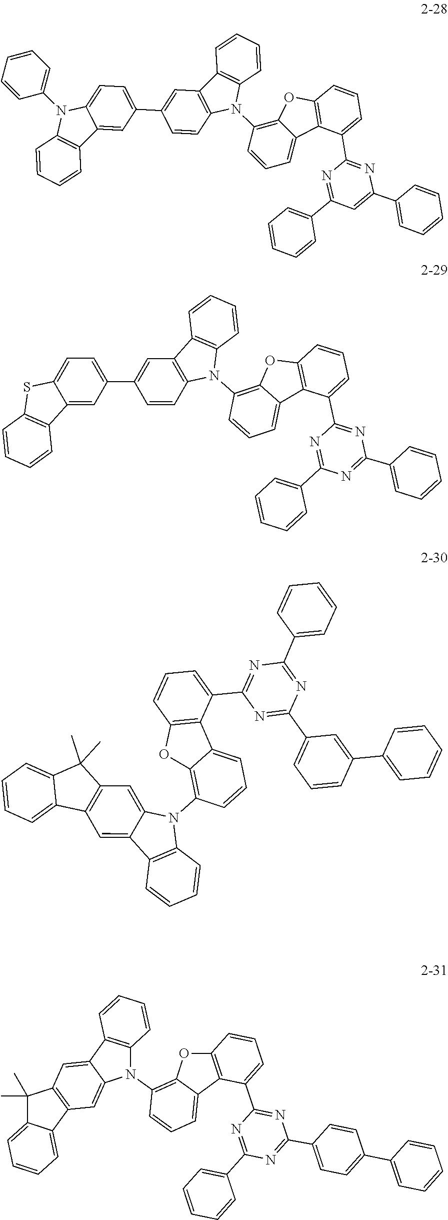 [SMILES: c1ccc(-c2cc(-c3ccccc3)nc(-c3cccc4oc5c(-n6c7ccccc7c7cc(-c8ccc9c(c8)c8ccccc8n9-c8ccccc8)ccc76)cccc5c34)n2)cc1 ; c1ccc(-c2nc(-c3ccccc3)nc(-c3cccc4oc5c(-n6c7ccccc7c7cc(-c8ccc9sc%10ccccc%10c9c8)ccc76)cccc5c34)n2)cc1 ; CC1(C)c2ccccc2-c2cc3c4ccccc4n(-c4cccc5c4oc4cccc(-c6nc(-c7ccccc7)nc(-c7cccc(-c8ccccc8)c7)n6)c45)c3cc21 ; CC1(C)c2ccccc2-c2cc3c(cc21)c1ccccc1n3-c1cccc2c1oc1cccc(-c3nc(-c4ccccc4)nc(-c4ccc(-c5ccccc5)cc4)n3)c12]

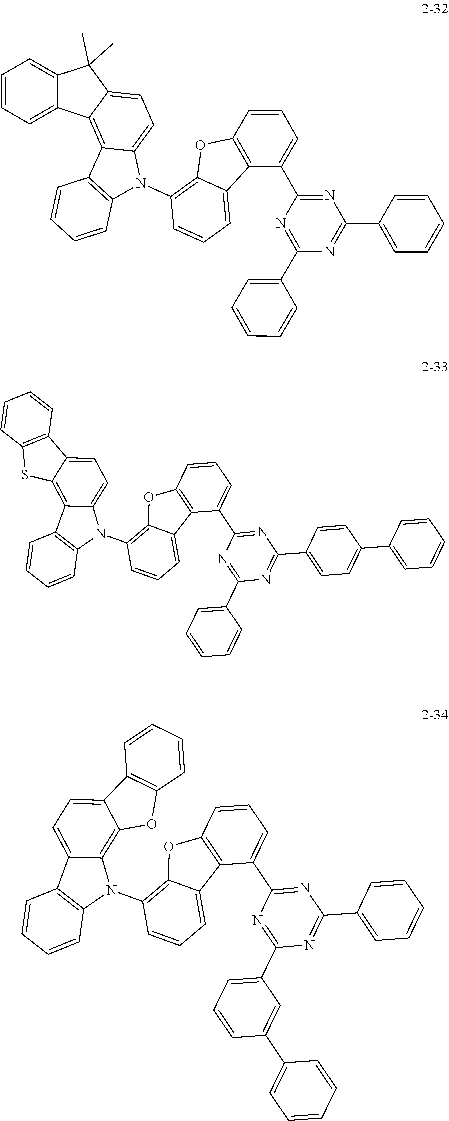 [SMILES: CC1(C)c2ccccc2-c2c1ccc1c2c2ccccc2n1-c1cccc2c1oc1cccc(-c3nc(-c4ccccc4)nc(-c4ccccc4)n3)c12 ; c1ccc(-c2ccc(-c3nc(-c4ccccc4)nc(-c4cccc5oc6c(-n7c8ccccc8c8c9sc%10ccccc%10c9ccc87)cccc6c45)n3)cc2)cc1 ; c1ccc(-c2cccc(-c3nc(-c4ccccc4)nc(-c4cccc5oc6c(-n7c8ccccc8c8ccc9c%10ccccc%10oc9c87)cccc6c45)n3)c2)cc1]

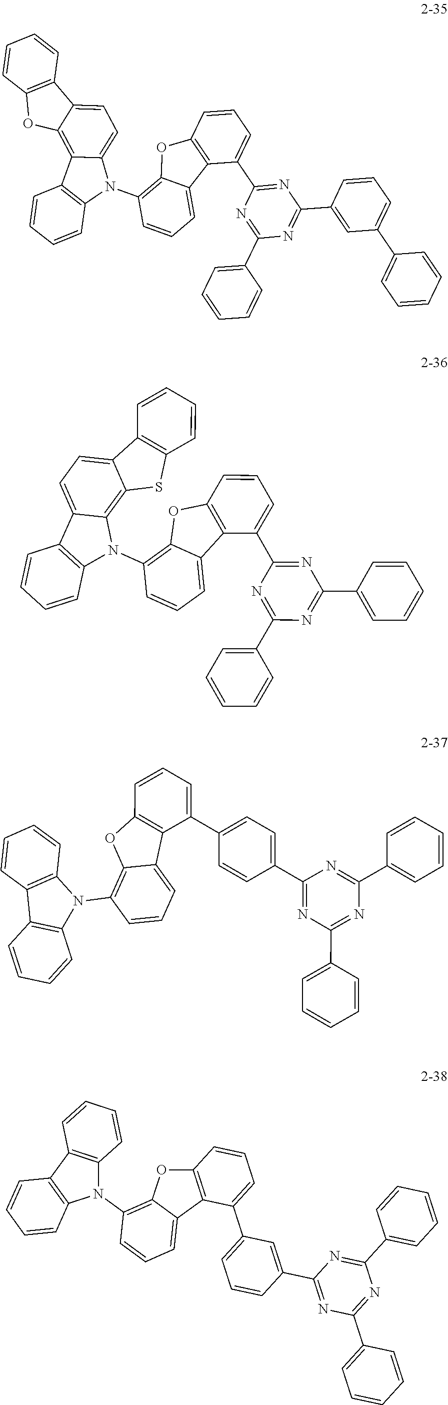 [SMILES: c1ccc(-c2cccc(-c3nc(-c4ccccc4)nc(-c4cccc5oc6c(-n7c8ccccc8c8c9oc%10ccccc%10c9ccc87)cccc6c45)n3)c2)cc1 ; c1ccc(-c2nc(-c3ccccc3)nc(-c3cccc4oc5c(-n6c7ccccc7c7ccc8c9ccccc9sc8c76)cccc5c34)n2)cc1 ; c1ccc(-c2nc(-c3ccccc3)nc(-c3ccc(-c4cccc5oc6c(-n7c8ccccc8c8ccccc87)cccc6c45)cc3)n2)cc1 ; c1ccc(-c2nc(-c3ccccc3)nc(-c3cccc(-c4cccc5oc6c(-n7c8ccccc8c8ccccc87)cccc6c45)c3)n2)cc1]

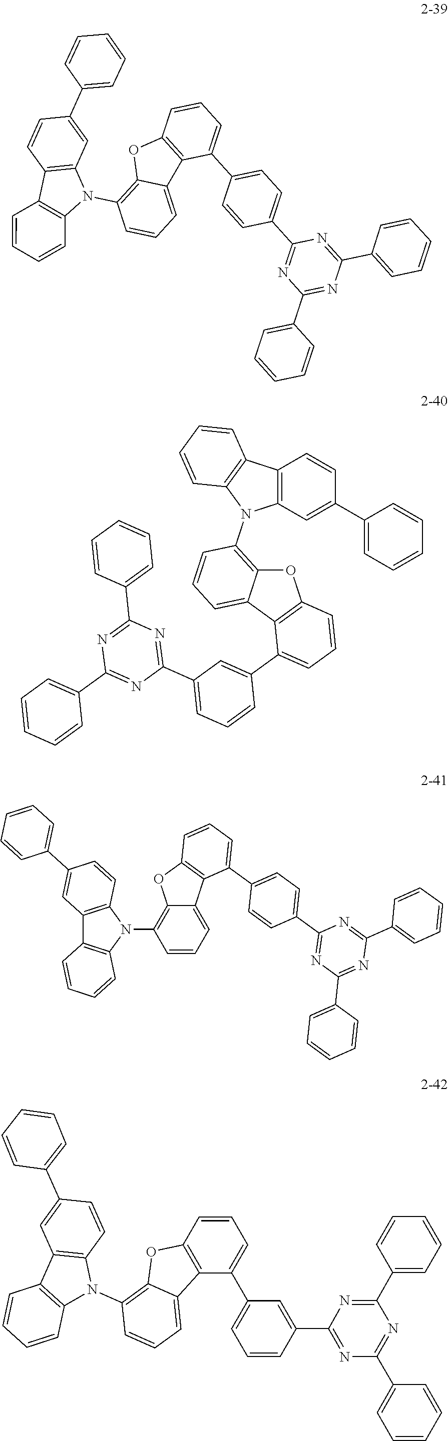 [SMILES: c1ccc(-c2ccc3c4ccccc4n(-c4cccc5c4oc4cccc(-c6ccc(-c7nc(-c8ccccc8)nc(-c8ccccc8)n7)cc6)c45)c3c2)cc1 ; c1ccc(-c2ccc3c4ccccc4n(-c4cccc5c4oc4cccc(-c6cccc(-c7nc(-c8ccccc8)nc(-c8ccccc8)n7)c6)c45)c3c2)cc1 ; c1ccc(-c2ccc3c(c2)c2ccccc2n3-c2cccc3c2oc2cccc(-c4ccc(-c5nc(-c6ccccc6)nc(-c6ccccc6)n5)cc4)c23)cc1 ; c1ccc(-c2ccc3c(c2)c2ccccc2n3-c2cccc3c2oc2cccc(-c4cccc(-c5nc(-c6ccccc6)nc(-c6ccccc6)n5)c4)c23)cc1]

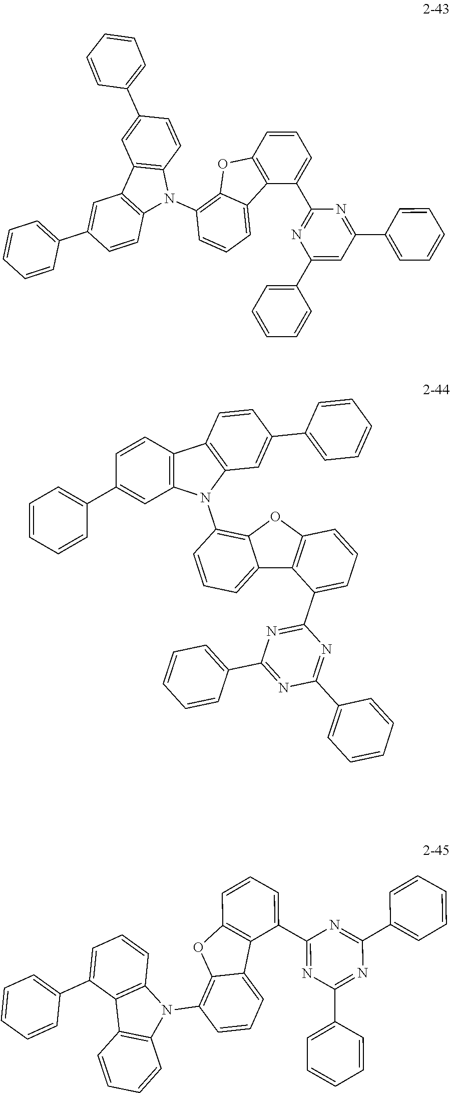 [SMILES: c1ccc(-c2ccc3c(c2)c2cc(-c4ccccc4)ccc2n3-c2cccc3c2oc2cccc(-c4nc(-c5ccccc5)cc(-c5ccccc5)n4)c23)cc1 ; c1ccc(-c2ccc3c4ccc(-c5ccccc5)cc4n(-c4cccc5c4oc4cccc(-c6nc(-c7ccccc7)nc(-c7ccccc7)n6)c45)c3c2)cc1 ; c1ccc(-c2nc(-c3ccccc3)nc(-c3cccc4oc5c(-n6c7ccccc7c7c(-c8ccccc8)cccc76)cccc5c34)n2)cc1]

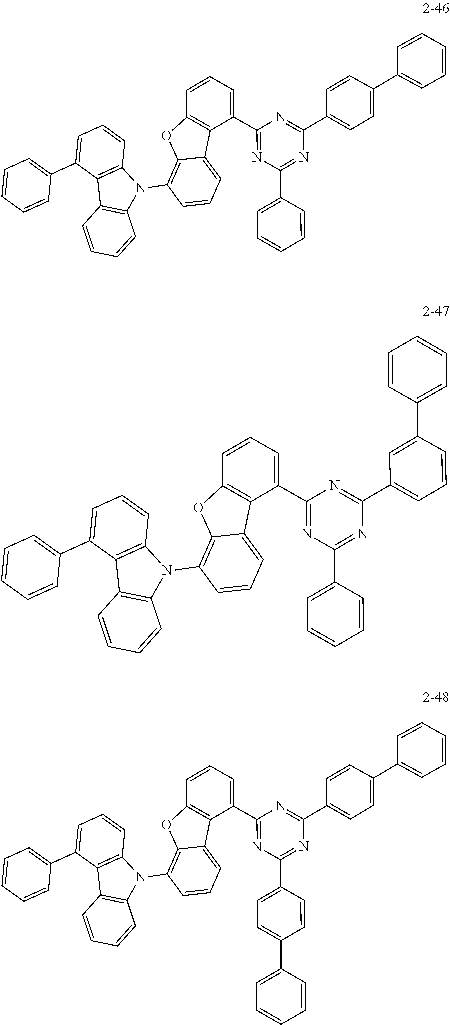 [SMILES: c1ccc(-c2ccc(-c3nc(-c4ccccc4)nc(-c4cccc5oc6c(-n7c8ccccc8c8c(-c9ccccc9)cccc87)cccc6c45)n3)cc2)cc1 ; c1ccc(-c2cccc(-c3nc(-c4ccccc4)nc(-c4cccc5oc6c(-n7c8ccccc8c8c(-c9ccccc9)cccc87)cccc6c45)n3)c2)cc1 ; c1ccc(-c2ccc(-c3nc(-c4ccc(-c5ccccc5)cc4)nc(-c4cccc5oc6c(-n7c8ccccc8c8c(-c9ccccc9)cccc87)cccc6c45)n3)cc2)cc1]

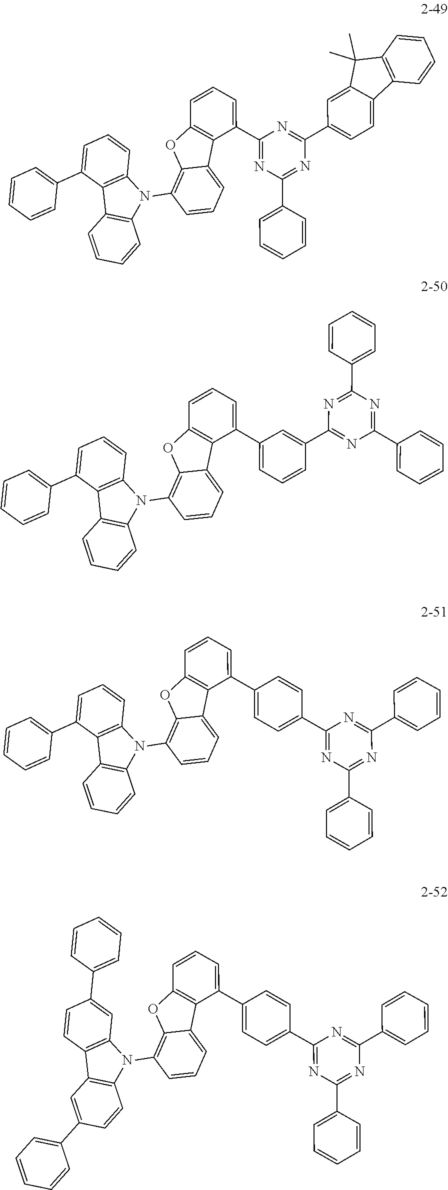 [SMILES: CC1(C)c2ccccc2-c2ccc(-c3nc(-c4ccccc4)nc(-c4cccc5oc6c(-n7c8ccccc8c8c(-c9ccccc9)cccc87)cccc6c45)n3)cc21 ; c1ccc(-c2nc(-c3ccccc3)nc(-c3cccc(-c4cccc5oc6c(-n7c8ccccc8c8c(-c9ccccc9)cccc87)cccc6c45)c3)n2)cc1 ; c1ccc(-c2nc(-c3ccccc3)nc(-c3ccc(-c4cccc5oc6c(-n7c8ccccc8c8c(-c9ccccc9)cccc87)cccc6c45)cc3)n2)cc1 ; c1ccc(-c2ccc3c(c2)c2ccc(-c4ccccc4)cc2n3-c2cccc3c2oc2cccc(-c4ccc(-c5nc(-c6ccccc6)nc(-c6ccccc6)n5)cc4)c23)cc1]

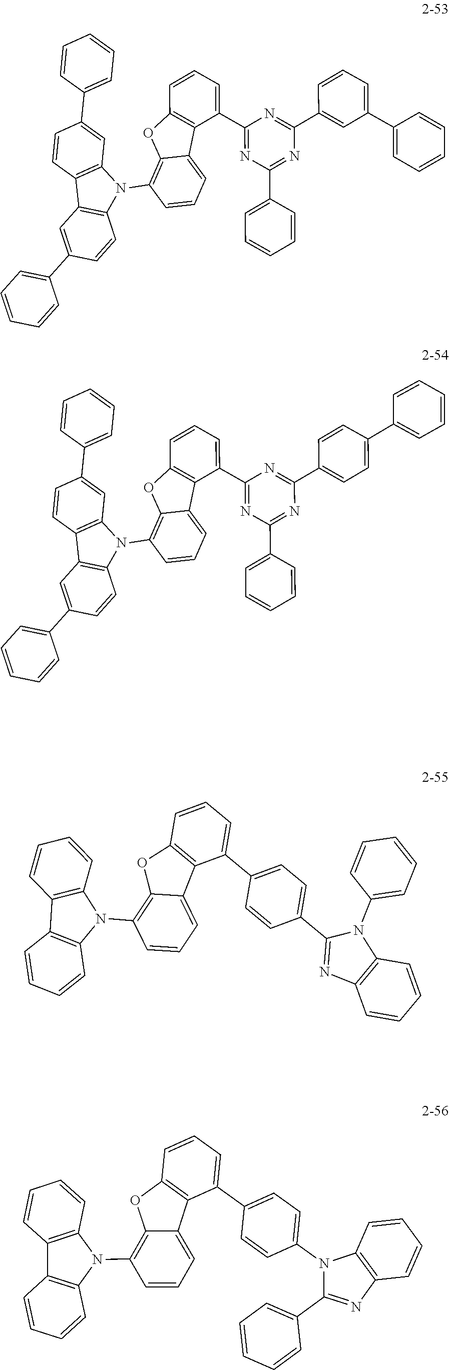 [SMILES: c1ccc(-c2cccc(-c3nc(-c4ccccc4)nc(-c4cccc5oc6c(-n7c8ccc(-c9ccccc9)cc8c8ccc(-c9ccccc9)cc87)cccc6c45)n3)c2)cc1 ; c1ccc(-c2ccc(-c3nc(-c4ccccc4)nc(-c4cccc5oc6c(-n7c8ccc(-c9ccccc9)cc8c8ccc(-c9ccccc9)cc87)cccc6c45)n3)cc2)cc1 ; c1ccc(-n2c(-c3ccc(-c4cccc5oc6c(-n7c8ccccc8c8ccccc87)cccc6c45)cc3)nc3ccccc32)cc1 ; c1ccc(-c2nc3ccccc3n2-c2ccc(-c3cccc4oc5c(-n6c7ccccc7c7ccccc76)cccc5c34)cc2)cc1]

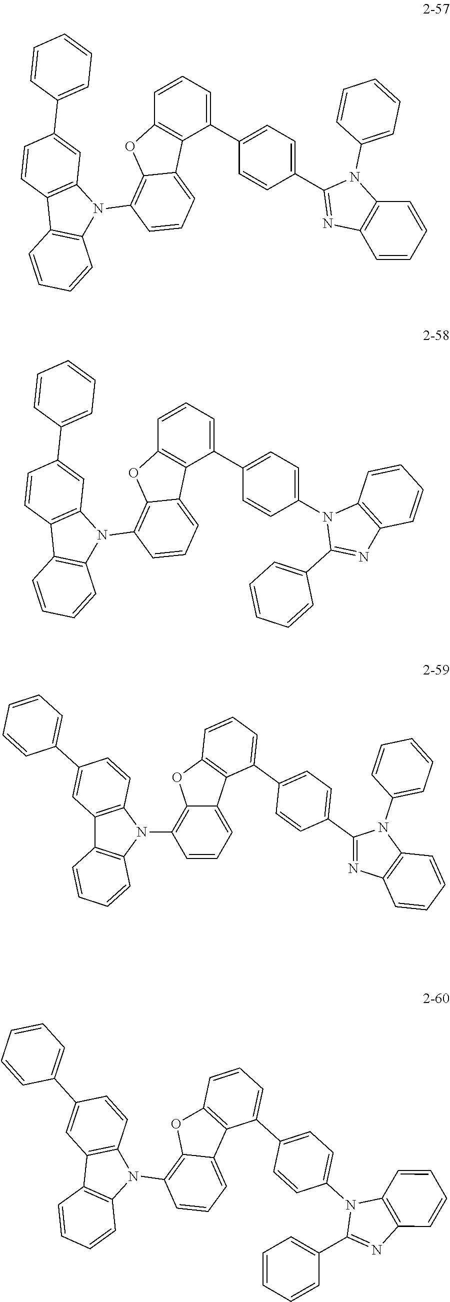 [SMILES: c1ccc(-c2ccc3c4ccccc4n(-c4cccc5c4oc4cccc(-c6ccc(-c7nc8ccccc8n7-c7ccccc7)cc6)c45)c3c2)cc1 ; c1ccc(-c2ccc3c4ccccc4n(-c4cccc5c4oc4cccc(-c6ccc(-n7c(-c8ccccc8)nc8ccccc87)cc6)c45)c3c2)cc1 ; c1ccc(-c2ccc3c(c2)c2ccccc2n3-c2cccc3c2oc2cccc(-c4ccc(-c5nc6ccccc6n5-c5ccccc5)cc4)c23)cc1 ; c1ccc(-c2ccc3c(c2)c2ccccc2n3-c2cccc3c2oc2cccc(-c4ccc(-n5c(-c6ccccc6)nc6ccccc65)cc4)c23)cc1]

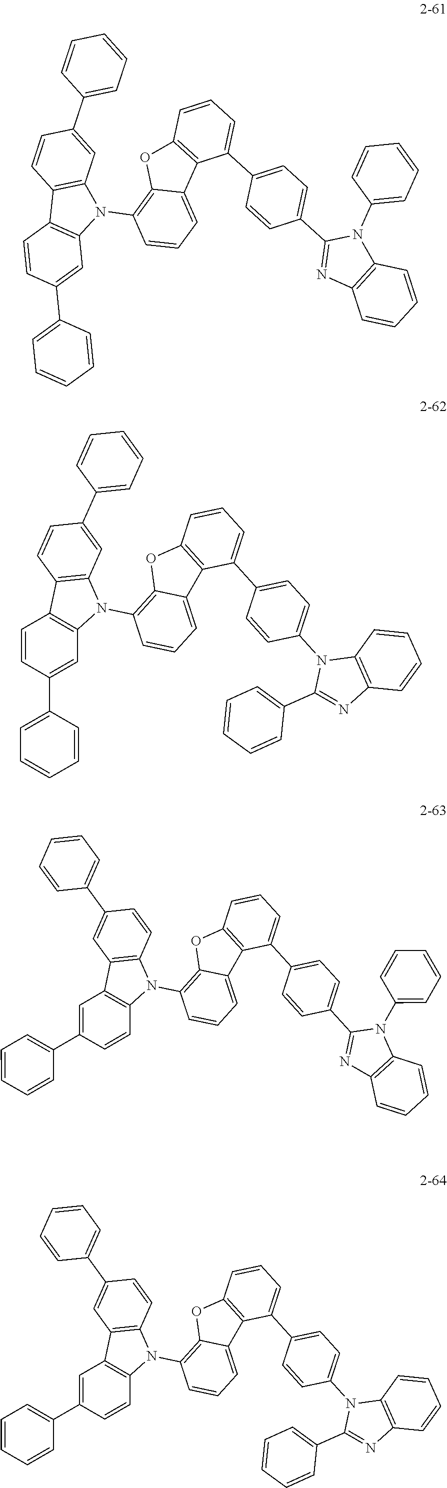 [SMILES: c1ccc(-c2ccc3c4ccc(-c5ccccc5)cc4n(-c4cccc5c4oc4cccc(-c6ccc(-c7nc8ccccc8n7-c7ccccc7)cc6)c45)c3c2)cc1 ; c1ccc(-c2ccc3c4ccc(-c5ccccc5)cc4n(-c4cccc5c4oc4cccc(-c6ccc(-n7c(-c8ccccc8)nc8ccccc87)cc6)c45)c3c2)cc1 ; c1ccc(-c2ccc3c(c2)c2cc(-c4ccccc4)ccc2n3-c2cccc3c2oc2cccc(-c4ccc(-c5nc6ccccc6n5-c5ccccc5)cc4)c23)cc1 ; c1ccc(-c2ccc3c(c2)c2cc(-c4ccccc4)ccc2n3-c2cccc3c2oc2cccc(-c4ccc(-n5c(-c6ccccc6)nc6ccccc65)cc4)c23)cc1]

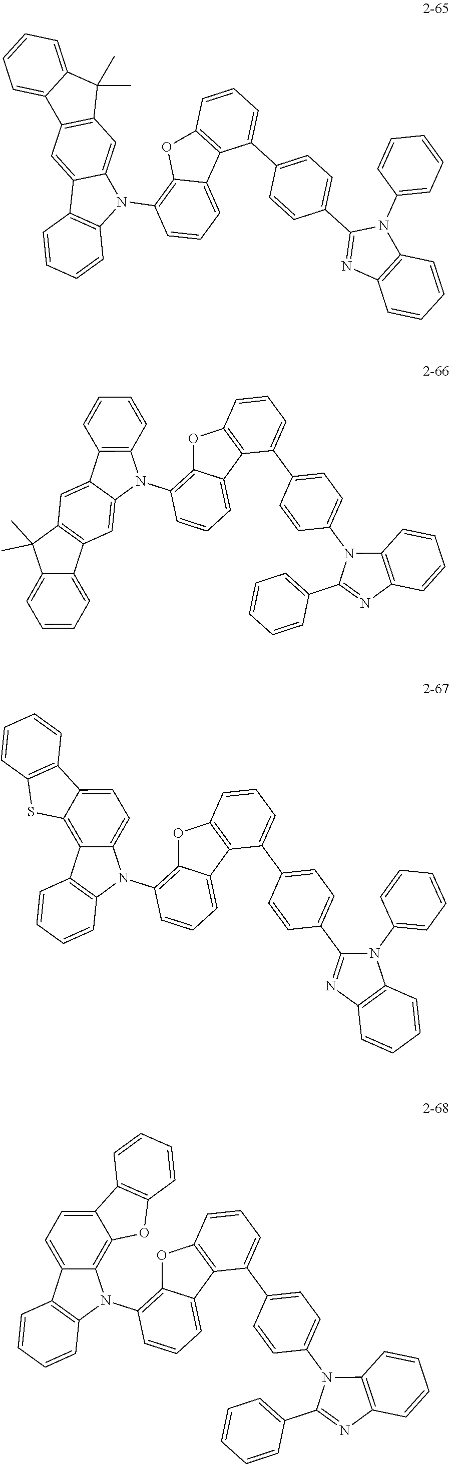 [SMILES: CC1(C)c2ccccc2-c2cc3c4ccccc4n(-c4cccc5c4oc4cccc(-c6ccc(-c7nc8ccccc8n7-c7ccccc7)cc6)c45)c3cc21 ; CC1(C)c2ccccc2-c2cc3c(cc21)c1ccccc1n3-c1cccc2c1oc1cccc(-c3ccc(-n4c(-c5ccccc5)nc5ccccc54)cc3)c12 ; c1ccc(-n2c(-c3ccc(-c4cccc5oc6c(-n7c8ccccc8c8c9sc%10ccccc%10c9ccc87)cccc6c45)cc3)nc3ccccc32)cc1 ; c1ccc(-c2nc3ccccc3n2-c2ccc(-c3cccc4oc5c(-n6c7ccccc7c7ccc8c9ccccc9oc8c76)cccc5c34)cc2)cc1]

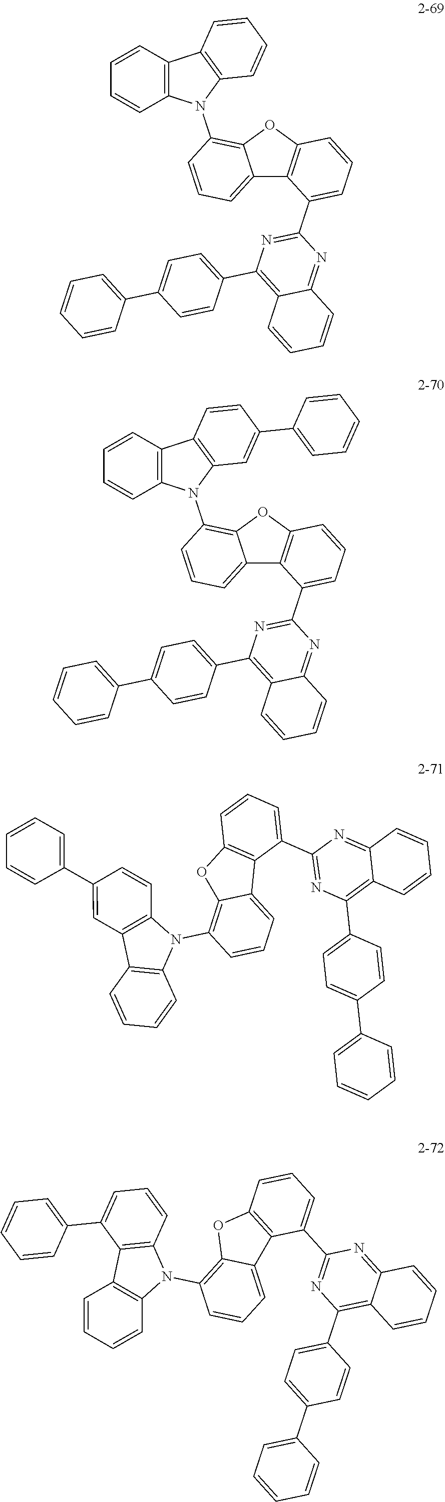 [SMILES: c1ccc(-c2ccc(-c3nc(-c4cccc5oc6c(-n7c8ccccc8c8ccccc87)cccc6c45)nc4ccccc34)cc2)cc1 ; c1ccc(-c2ccc(-c3nc(-c4cccc5oc6c(-n7c8ccccc8c8ccc(-c9ccccc9)cc87)cccc6c45)nc4ccccc34)cc2)cc1 ; c1ccc(-c2ccc(-c3nc(-c4cccc5oc6c(-n7c8ccccc8c8cc(-c9ccccc9)ccc87)cccc6c45)nc4ccccc34)cc2)cc1 ; c1ccc(-c2ccc(-c3nc(-c4cccc5oc6c(-n7c8ccccc8c8c(-c9ccccc9)cccc87)cccc6c45)nc4ccccc34)cc2)cc1]

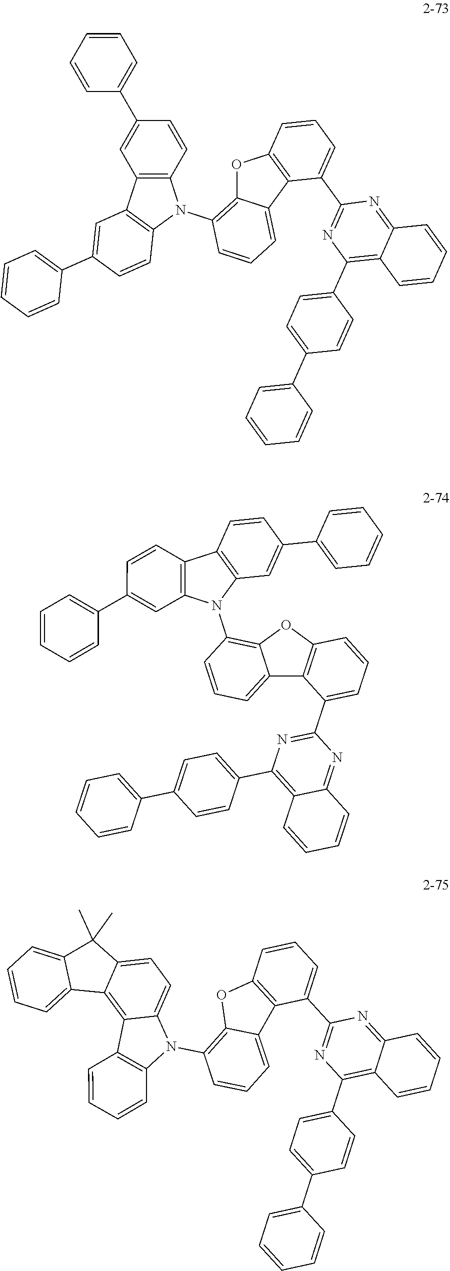 [SMILES: c1ccc(-c2ccc(-c3nc(-c4cccc5oc6c(-n7c8ccc(-c9ccccc9)cc8c8cc(-c9ccccc9)ccc87)cccc6c45)nc4ccccc34)cc2)cc1 ; c1ccc(-c2ccc(-c3nc(-c4cccc5oc6c(-n7c8cc(-c9ccccc9)ccc8c8ccc(-c9ccccc9)cc87)cccc6c45)nc4ccccc34)cc2)cc1 ; CC1(C)c2ccccc2-c2c1ccc1c2c2ccccc2n1-c1cccc2c1oc1cccc(-c3nc(-c4ccc(-c5ccccc5)cc4)c4ccccc4n3)c12]

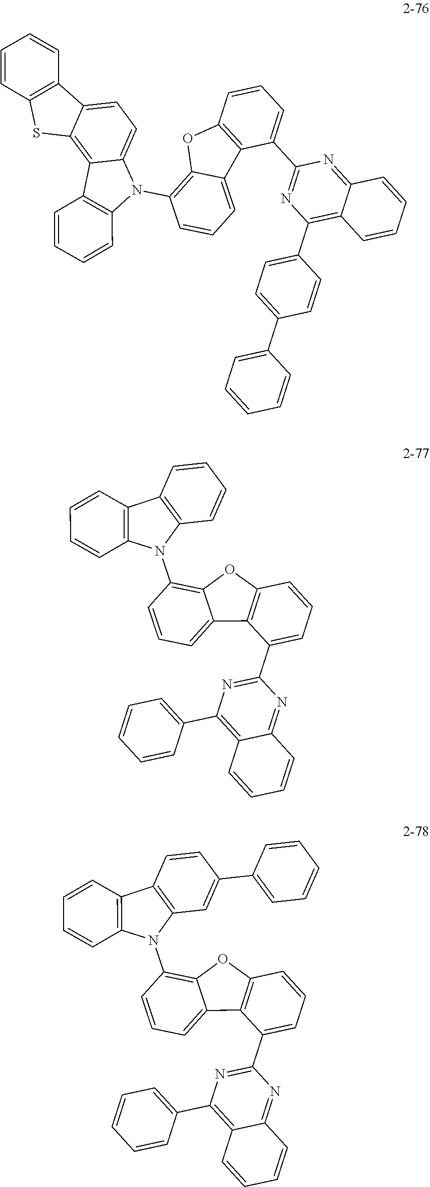 [SMILES: c1ccc(-c2ccc(-c3nc(-c4cccc5oc6c(-n7c8ccccc8c8c9sc%10ccccc%10c9ccc87)cccc6c45)nc4ccccc34)cc2)cc1 ; c1ccc(-c2nc(-c3cccc4oc5c(-n6c7ccccc7c7ccccc76)cccc5c34)nc3ccccc23)cc1 ; c1ccc(-c2ccc3c4ccccc4n(-c4cccc5c4oc4cccc(-c6nc(-c7ccccc7)c7ccccc7n6)c45)c3c2)cc1]

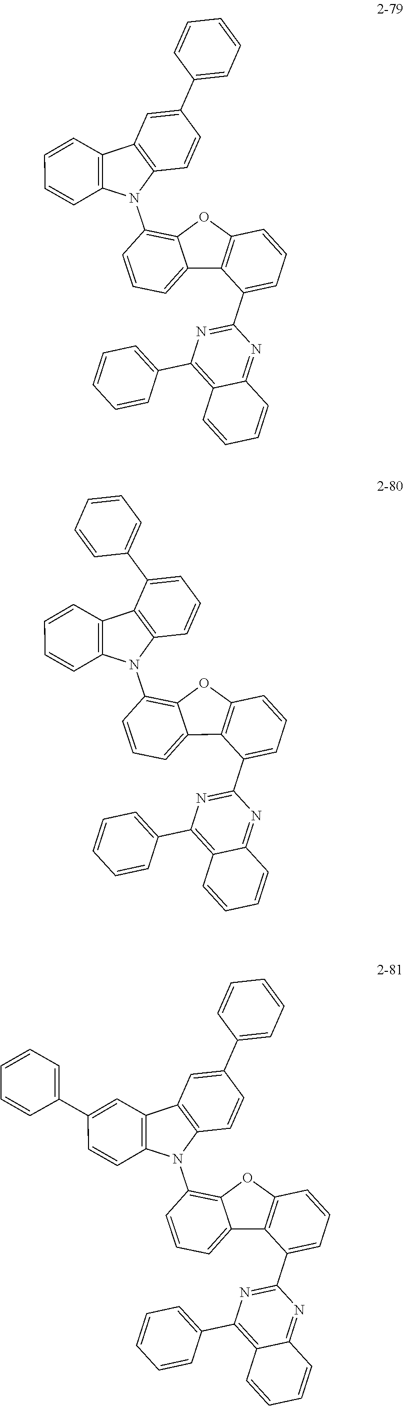 [SMILES: c1ccc(-c2ccc3c(c2)c2ccccc2n3-c2cccc3c2oc2cccc(-c4nc(-c5ccccc5)c5ccccc5n4)c23)cc1 ; c1ccc(-c2nc(-c3cccc4oc5c(-n6c7ccccc7c7c(-c8ccccc8)cccc76)cccc5c34)nc3ccccc23)cc1 ; c1ccc(-c2ccc3c(c2)c2cc(-c4ccccc4)ccc2n3-c2cccc3c2oc2cccc(-c4nc(-c5ccccc5)c5ccccc5n4)c23)cc1]

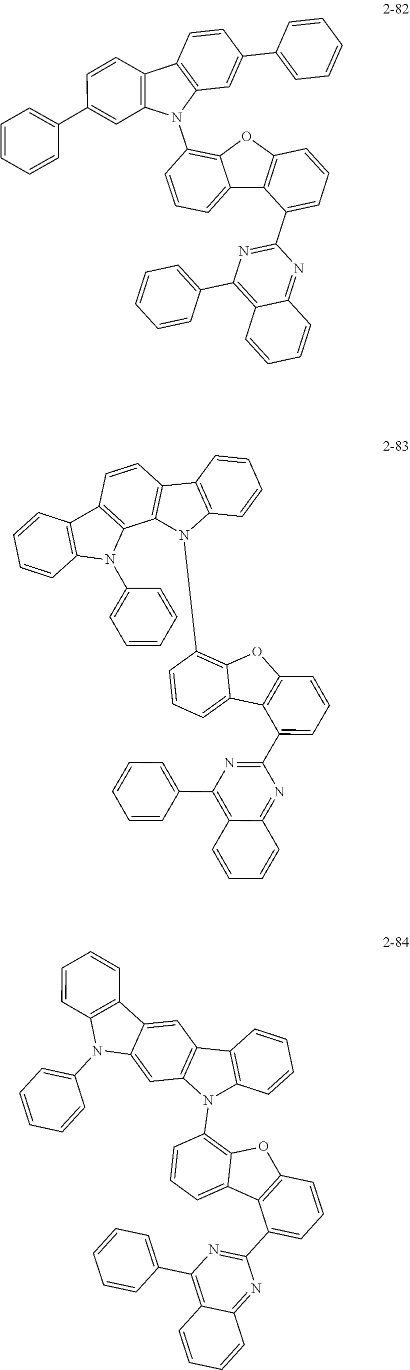 [SMILES: c1ccc(-c2ccc3c4ccc(-c5ccccc5)cc4n(-c4cccc5c4oc4cccc(-c6nc(-c7ccccc7)c7ccccc7n6)c45)c3c2)cc1 ; c1ccc(-c2nc(-c3cccc4oc5c(-n6c7ccccc7c7ccc8c9ccccc9n(-c9ccccc9)c8c76)cccc5c34)nc3ccccc23)cc1 ; c1ccc(-c2nc(-c3cccc4oc5c(-n6c7ccccc7c7cc8c9ccccc9n(-c9ccccc9)c8cc76)cccc5c34)nc3ccccc23)cc1]

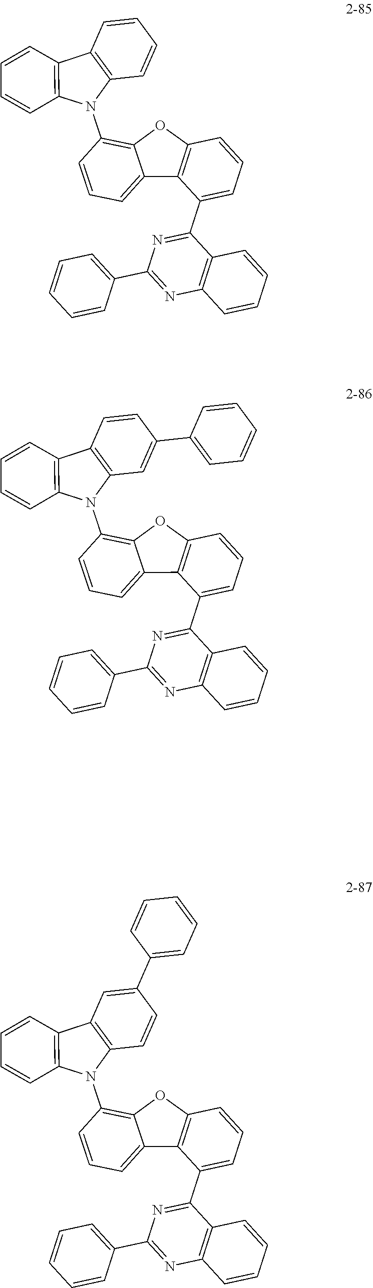 [SMILES: c1ccc(-c2nc(-c3cccc4oc5c(-n6c7ccccc7c7ccccc76)cccc5c34)c3ccccc3n2)cc1 ; c1ccc(-c2ccc3c4ccccc4n(-c4cccc5c4oc4cccc(-c6nc(-c7ccccc7)nc7ccccc67)c45)c3c2)cc1 ; c1ccc(-c2ccc3c(c2)c2ccccc2n3-c2cccc3c2oc2cccc(-c4nc(-c5ccccc5)nc5ccccc45)c23)cc1]

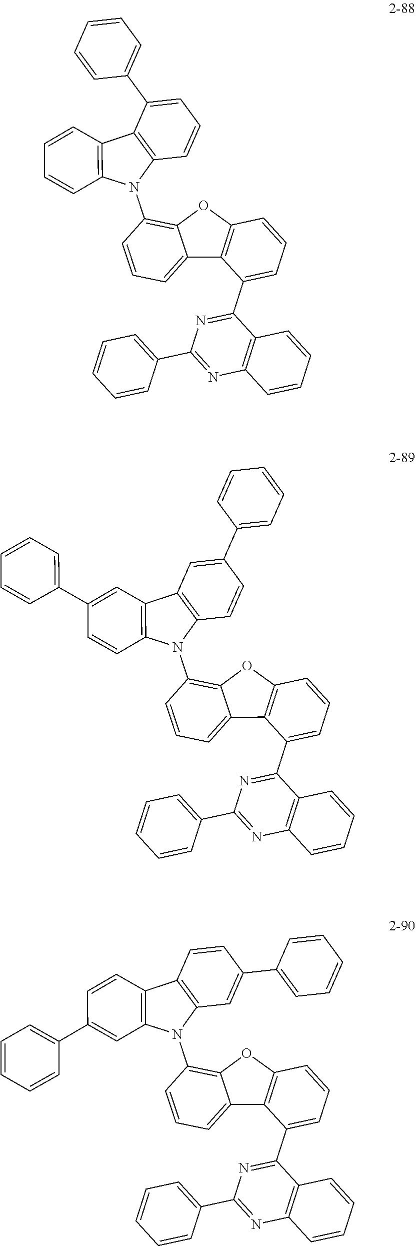 [SMILES: c1ccc(-c2nc(-c3cccc4oc5c(-n6c7ccccc7c7c(-c8ccccc8)cccc76)cccc5c34)c3ccccc3n2)cc1 ; c1ccc(-c2ccc3c(c2)c2cc(-c4ccccc4)ccc2n3-c2cccc3c2oc2cccc(-c4nc(-c5ccccc5)nc5ccccc45)c23)cc1 ; c1ccc(-c2ccc3c4ccc(-c5ccccc5)cc4n(-c4cccc5c4oc4cccc(-c6nc(-c7ccccc7)nc7ccccc67)c45)c3c2)cc1]

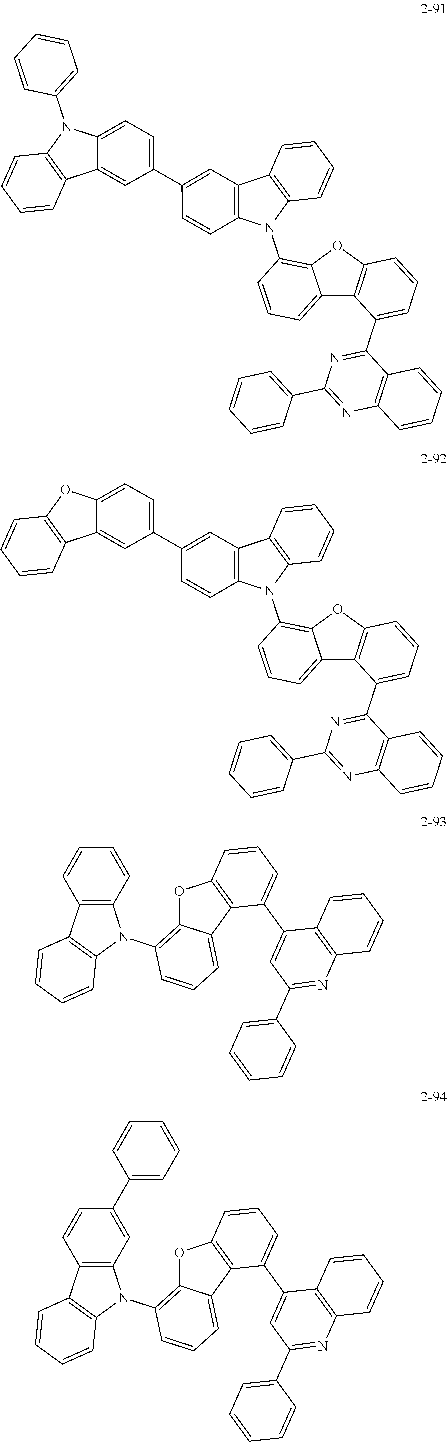 [SMILES: c1ccc(-c2nc(-c3cccc4oc5c(-n6c7ccccc7c7cc(-c8ccc9c(c8)c8ccccc8n9-c8ccccc8)ccc76)cccc5c34)c3ccccc3n2)cc1 ; c1ccc(-c2nc(-c3cccc4oc5c(-n6c7ccccc7c7cc(-c8ccc9oc%10ccccc%10c9c8)ccc76)cccc5c34)c3ccccc3n2)cc1 ; c1ccc(-c2cc(-c3cccc4oc5c(-n6c7ccccc7c7ccccc76)cccc5c34)c3ccccc3n2)cc1 ; c1ccc(-c2ccc3c4ccccc4n(-c4cccc5c4oc4cccc(-c6cc(-c7ccccc7)nc7ccccc67)c45)c3c2)cc1]

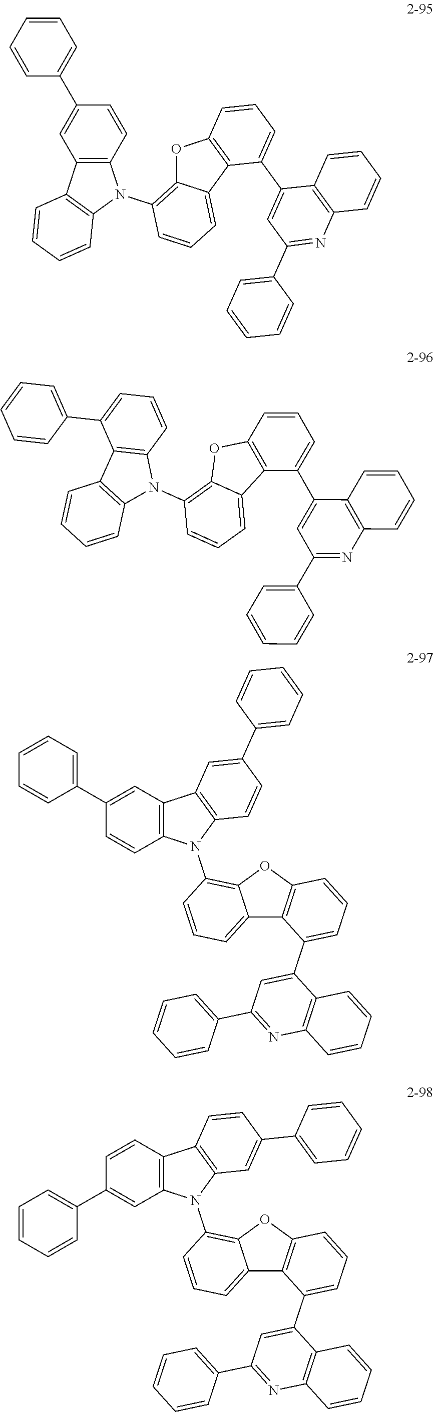

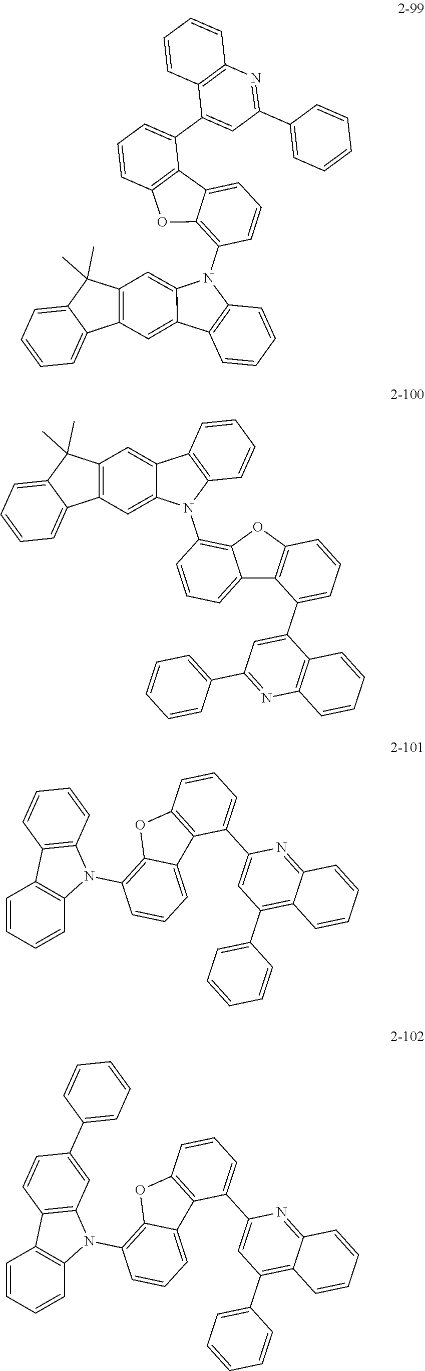 [SMILES: CC1(C)c2ccccc2-c2cc3c4ccccc4n(-c4cccc5c4oc4cccc(-c6cc(-c7ccccc7)nc7ccccc67)c45)c3cc21 ; CC1(C)c2ccccc2-c2cc3c(cc21)c1ccccc1n3-c1cccc2c1oc1cccc(-c3cc(-c4ccccc4)nc4ccccc34)c12 ; c1ccc(-c2cc(-c3cccc4oc5c(-n6c7ccccc7c7ccccc76)cccc5c34)nc3ccccc23)cc1 ; c1ccc(-c2ccc3c4ccccc4n(-c4cccc5c4oc4cccc(-c6cc(-c7ccccc7)c7ccccc7n6)c45)c3c2)cc1]

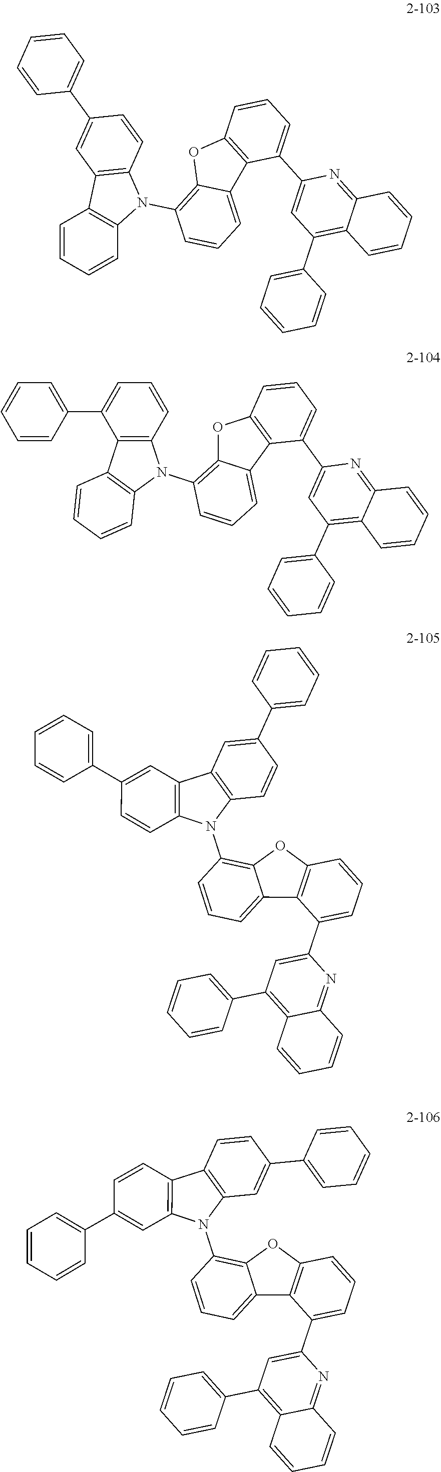

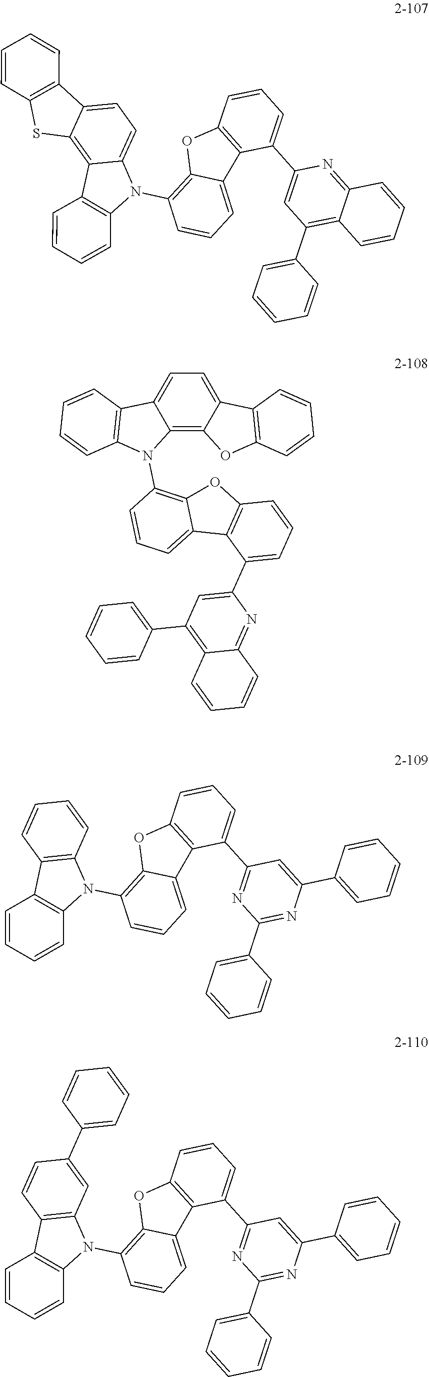 [SMILES: c1ccc(-c2cc(-c3cccc4oc5c(-n6c7ccccc7c7c8sc9ccccc9c8ccc76)cccc5c34)nc3ccccc23)cc1 ; c1ccc(-c2cc(-c3cccc4oc5c(-n6c7ccccc7c7ccc8c9ccccc9oc8c76)cccc5c34)nc3ccccc23)cc1 ; c1ccc(-c2cc(-c3cccc4oc5c(-n6c7ccccc7c7ccccc76)cccc5c34)nc(-c3ccccc3)n2)cc1 ; c1ccc(-c2ccc3c4ccccc4n(-c4cccc5c4oc4cccc(-c6cc(-c7ccccc7)nc(-c7ccccc7)n6)c45)c3c2)cc1]

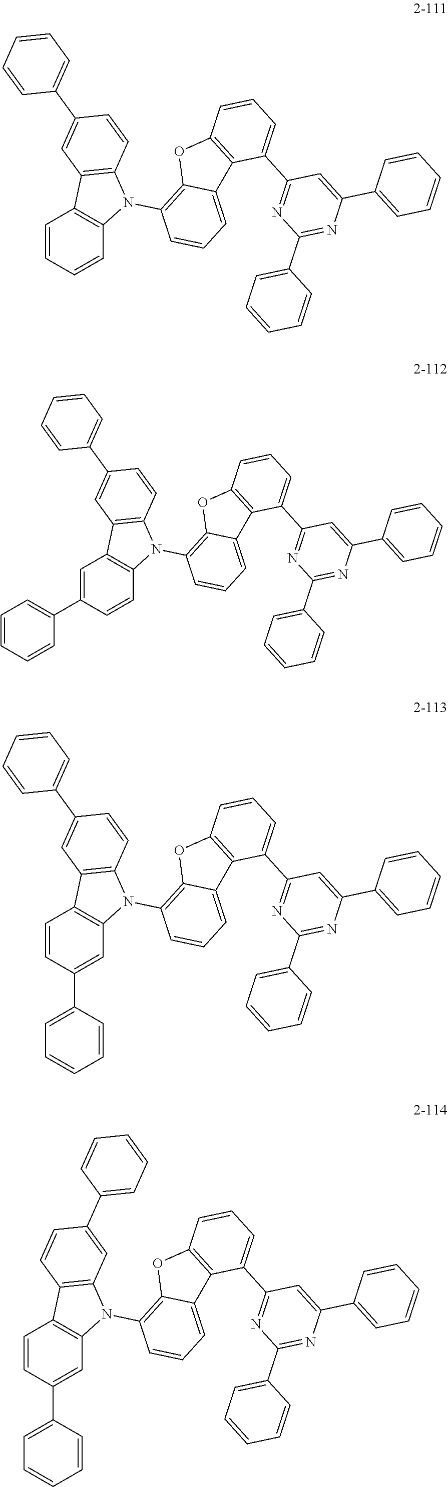 [SMILES: c1ccc(-c2ccc3c(c2)c2ccccc2n3-c2cccc3c2oc2cccc(-c4cc(-c5ccccc5)nc(-c5ccccc5)n4)c23)cc1 ; c1ccc(-c2ccc3c(c2)c2cc(-c4ccccc4)ccc2n3-c2cccc3c2oc2cccc(-c4cc(-c5ccccc5)nc(-c5ccccc5)n4)c23)cc1 ; c1ccc(-c2ccc3c(c2)c2ccc(-c4ccccc4)cc2n3-c2cccc3c2oc2cccc(-c4cc(-c5ccccc5)nc(-c5ccccc5)n4)c23)cc1 ; c1ccc(-c2ccc3c4ccc(-c5ccccc5)cc4n(-c4cccc5c4oc4cccc(-c6cc(-c7ccccc7)nc(-c7ccccc7)n6)c45)c3c2)cc1]

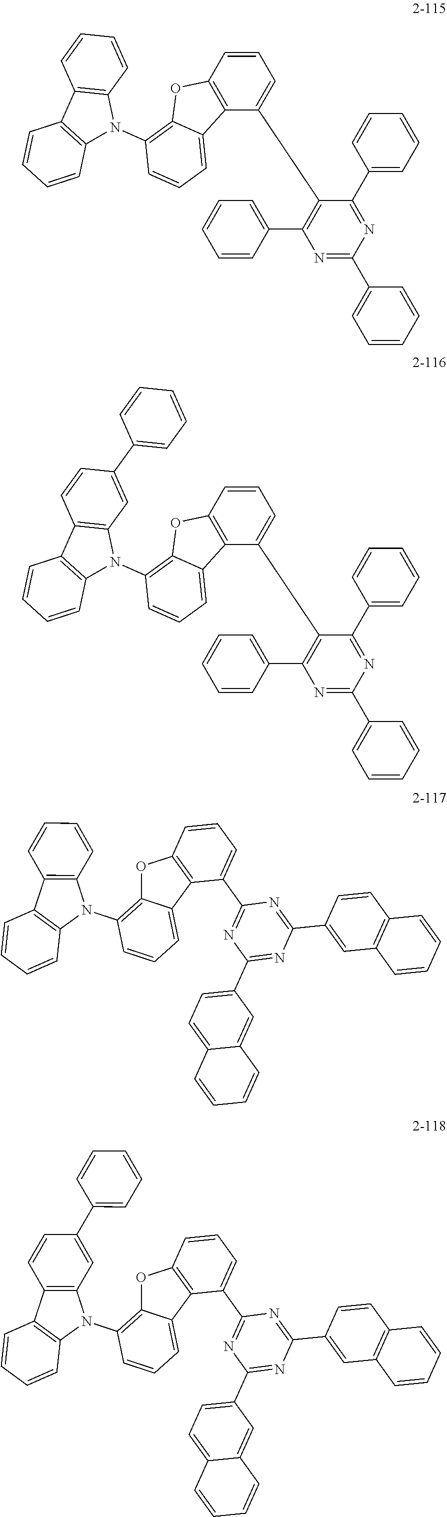 [SMILES: c1ccc(-c2nc(-c3ccccc3)c(-c3cccc4oc5c(-n6c7ccccc7c7ccccc76)cccc5c34)c(-c3ccccc3)n2)cc1 ; c1ccc(-c2ccc3c4ccccc4n(-c4cccc5c4oc4cccc(-c6c(-c7ccccc7)nc(-c7ccccc7)nc6-c6ccccc6)c45)c3c2)cc1 ; c1ccc2cc(-c3nc(-c4ccc5ccccc5c4)nc(-c4cccc5oc6c(-n7c8ccccc8c8ccccc87)cccc6c45)n3)ccc2c1 ; c1ccc(-c2ccc3c4ccccc4n(-c4cccc5c4oc4cccc(-c6nc(-c7ccc8ccccc8c7)nc(-c7ccc8ccccc8c7)n6)c45)c3c2)cc1]

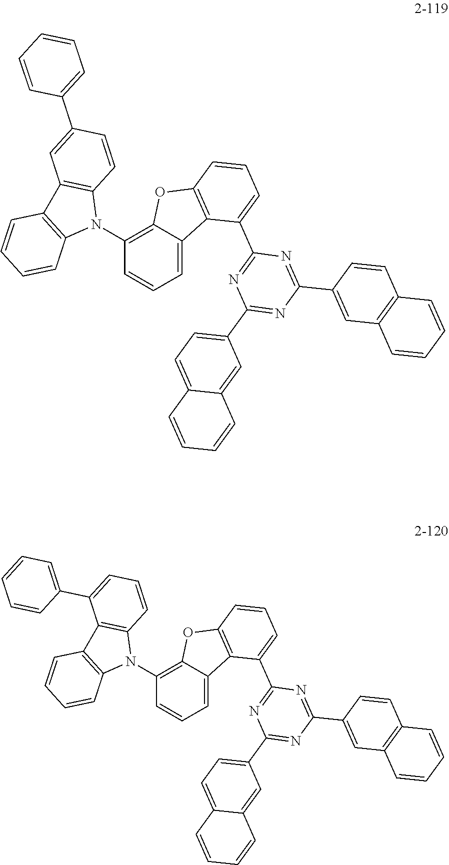 [SMILES: c1ccc(-c2ccc3c(c2)c2ccccc2n3-c2cccc3c2oc2cccc(-c4nc(-c5ccc6ccccc6c5)nc(-c5ccc6ccccc6c5)n4)c23)cc1 ; c1ccc(-c2cccc3c2c2ccccc2n3-c2cccc3c2oc2cccc(-c4nc(-c5ccc6ccccc6c5)nc(-c5ccc6ccccc6c5)n4)c23)cc1]

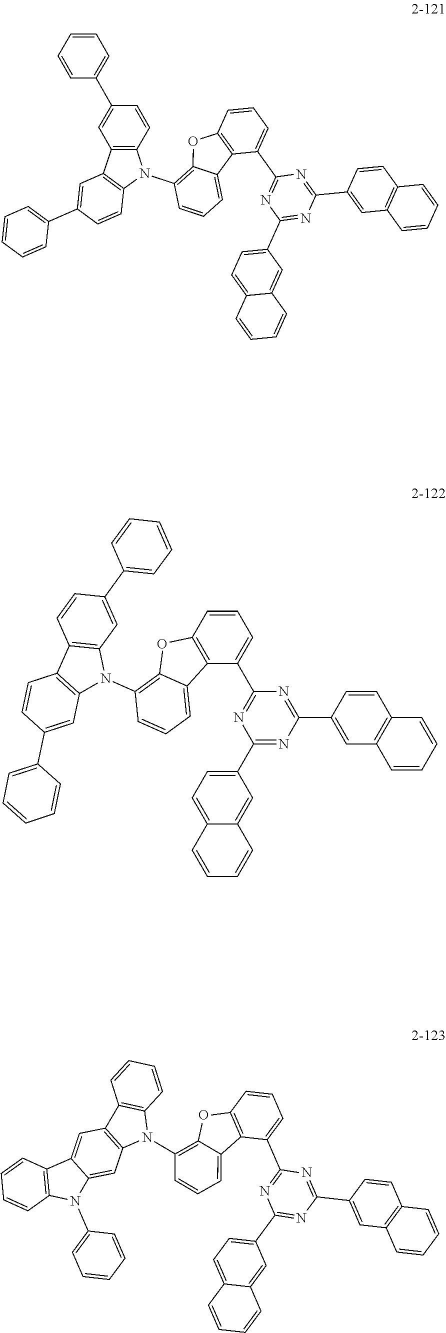 [SMILES: c1ccc(-c2ccc3c(c2)c2cc(-c4ccccc4)ccc2n3-c2cccc3c2oc2cccc(-c4nc(-c5ccc6ccccc6c5)nc(-c5ccc6ccccc6c5)n4)c23)cc1 ; c1ccc(-c2ccc3c4ccc(-c5ccccc5)cc4n(-c4cccc5c4oc4cccc(-c6nc(-c7ccc8ccccc8c7)nc(-c7ccc8ccccc8c7)n6)c45)c3c2)cc1 ; c1ccc(-n2c3ccccc3c3cc4c5ccccc5n(-c5cccc6c5oc5cccc(-c7nc(-c8ccc9ccccc9c8)nc(-c8ccc9ccccc9c8)n7)c56)c4cc32)cc1]

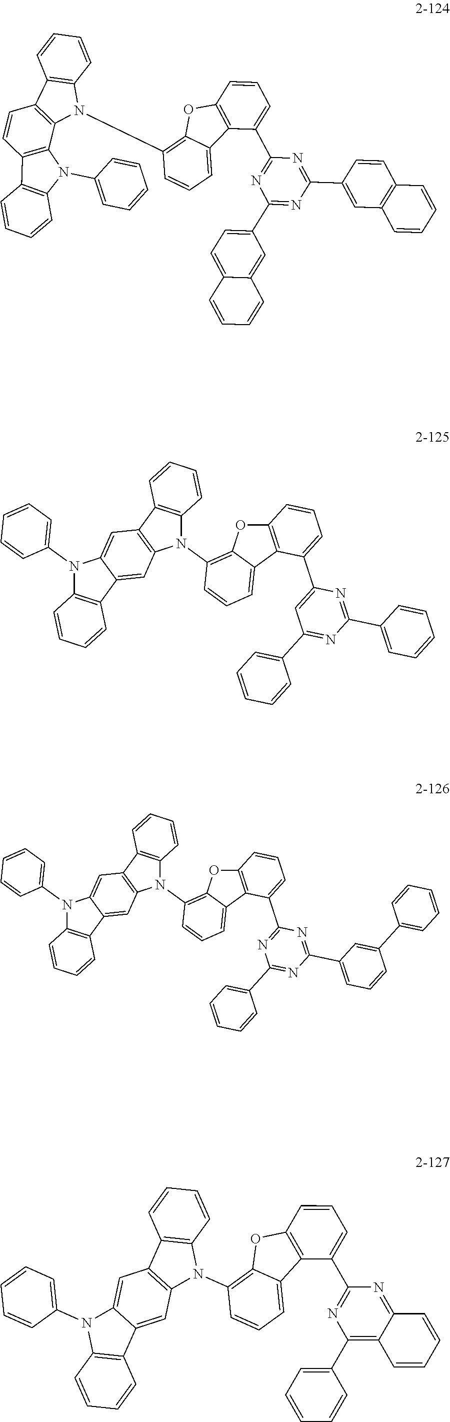 [SMILES: c1ccc(-n2c3ccccc3c3ccc4c5ccccc5n(-c5cccc6c5oc5cccc(-c7nc(-c8ccc9ccccc9c8)nc(-c8ccc9ccccc9c8)n7)c56)c4c32)cc1 ; c1ccc(-c2cc(-c3cccc4oc5c(-n6c7ccccc7c7cc8c(cc76)c6ccccc6n8-c6ccccc6)cccc5c34)nc(-c3ccccc3)n2)cc1 ; c1ccc(-c2cccc(-c3nc(-c4ccccc4)nc(-c4cccc5oc6c(-n7c8ccccc8c8cc9c(cc87)c7ccccc7n9-c7ccccc7)cccc6c45)n3)c2)cc1 ; c1ccc(-c2nc(-c3cccc4oc5c(-n6c7ccccc7c7cc8c(cc76)c6ccccc6n8-c6ccccc6)cccc5c34)nc3ccccc23)cc1]

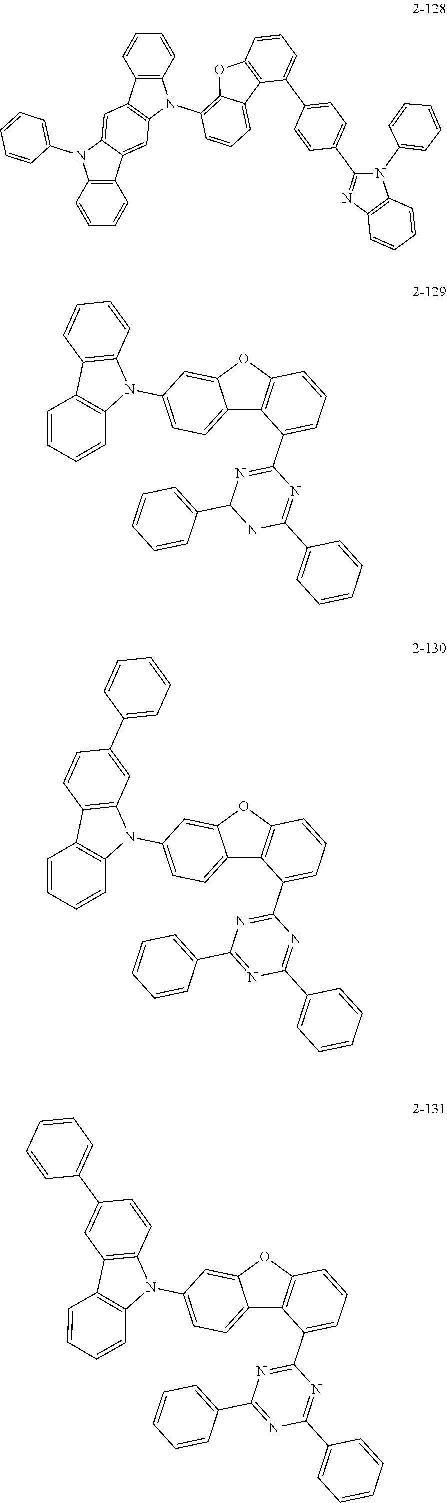

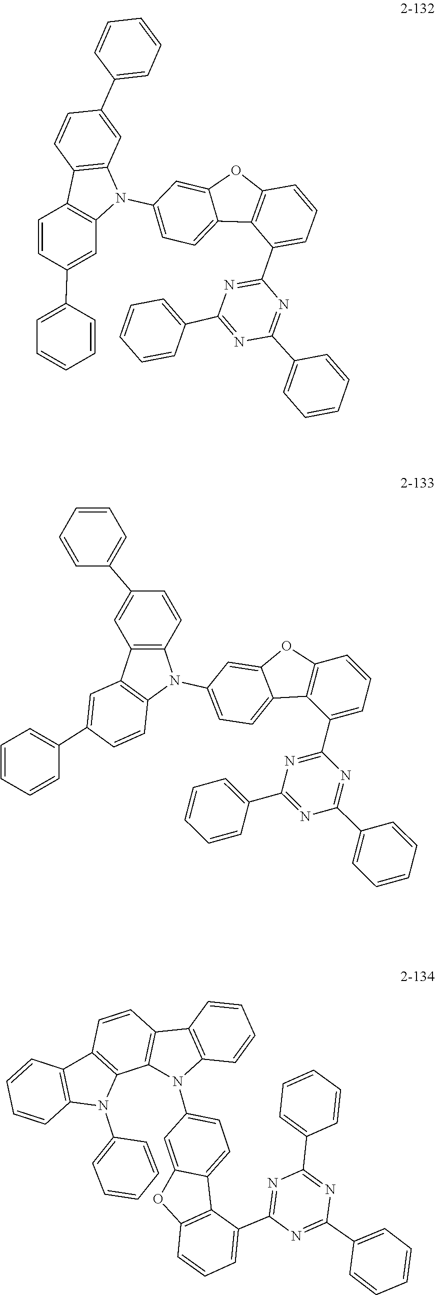 [SMILES: c1ccc(-c2ccc3c4ccc(-c5ccccc5)cc4n(-c4ccc5c(c4)oc4cccc(-c6nc(-c7ccccc7)nc(-c7ccccc7)n6)c45)c3c2)cc1 ; c1ccc(-c2ccc3c(c2)c2cc(-c4ccccc4)ccc2n3-c2ccc3c(c2)oc2cccc(-c4nc(-c5ccccc5)nc(-c5ccccc5)n4)c23)cc1 ; c1ccc(-c2nc(-c3ccccc3)nc(-c3cccc4oc5cc(-n6c7ccccc7c7ccc8c9ccccc9n(-c9ccccc9)c8c76)ccc5c34)n2)cc1]

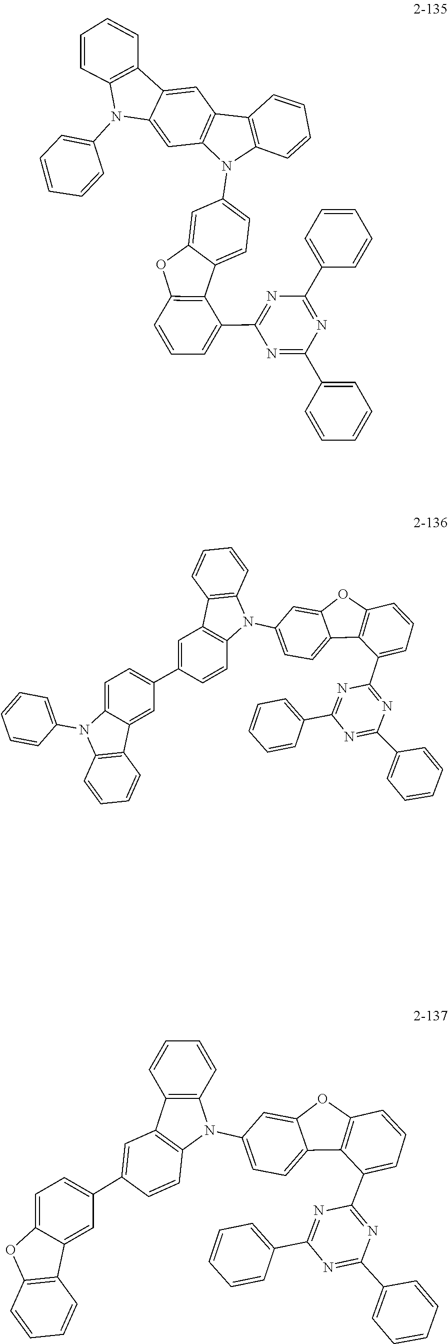 [SMILES: c1ccc(-c2nc(-c3ccccc3)nc(-c3cccc4oc5cc(-n6c7ccccc7c7cc8c9ccccc9n(-c9ccccc9)c8cc76)ccc5c34)n2)cc1 ; c1ccc(-c2nc(-c3ccccc3)nc(-c3cccc4oc5cc(-n6c7ccccc7c7cc(-c8ccc9c(c8)c8ccccc8n9-c8ccccc8)ccc76)ccc5c34)n2)cc1 ; c1ccc(-c2nc(-c3ccccc3)nc(-c3cccc4oc5cc(-n6c7ccccc7c7cc(-c8ccc9oc%10ccccc%10c9c8)ccc76)ccc5c34)n2)cc1]

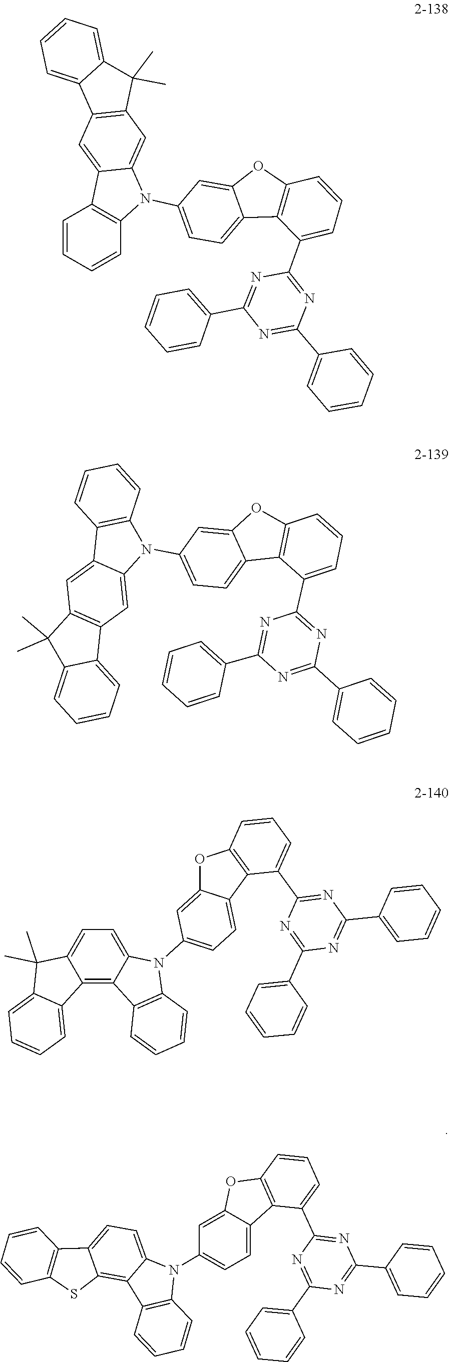 [SMILES: CC1(C)c2ccccc2-c2cc3c4ccccc4n(-c4ccc5c(c4)oc4cccc(-c6nc(-c7ccccc7)nc(-c7ccccc7)n6)c45)c3cc21 ; CC1(C)c2ccccc2-c2cc3c(cc21)c1ccccc1n3-c1ccc2c(c1)oc1cccc(-c3nc(-c4ccccc4)nc(-c4ccccc4)n3)c12 ; CC1(C)c2ccccc2-c2c1ccc1c2c2ccccc2n1-c1ccc2c(c1)oc1cccc(-c3nc(-c4ccccc4)nc(-c4ccccc4)n3)c12 ; c1ccc(-c2nc(-c3ccccc3)nc(-c3cccc4oc5cc(-n6c7ccccc7c7c8sc9ccccc9c8ccc76)ccc5c34)n2)cc1]

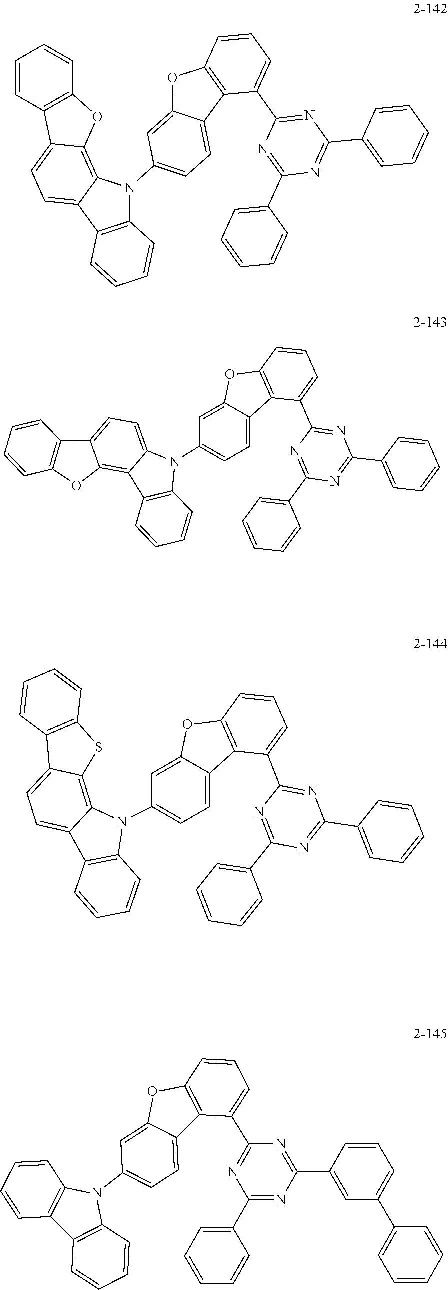 [SMILES: c1ccc(-c2nc(-c3ccccc3)nc(-c3cccc4oc5cc(-n6c7ccccc7c7ccc8c9ccccc9oc8c76)ccc5c34)n2)cc1 ; c1ccc(-c2nc(-c3ccccc3)nc(-c3cccc4oc5cc(-n6c7ccccc7c7c8oc9ccccc9c8ccc76)ccc5c34)n2)cc1 ; c1ccc(-c2nc(-c3ccccc3)nc(-c3cccc4oc5cc(-n6c7ccccc7c7ccc8c9ccccc9sc8c76)ccc5c34)n2)cc1 ; c1ccc(-c2cccc(-c3nc(-c4ccccc4)nc(-c4cccc5oc6cc(-n7c8ccccc8c8ccccc87)ccc6c45)n3)c2)cc1]

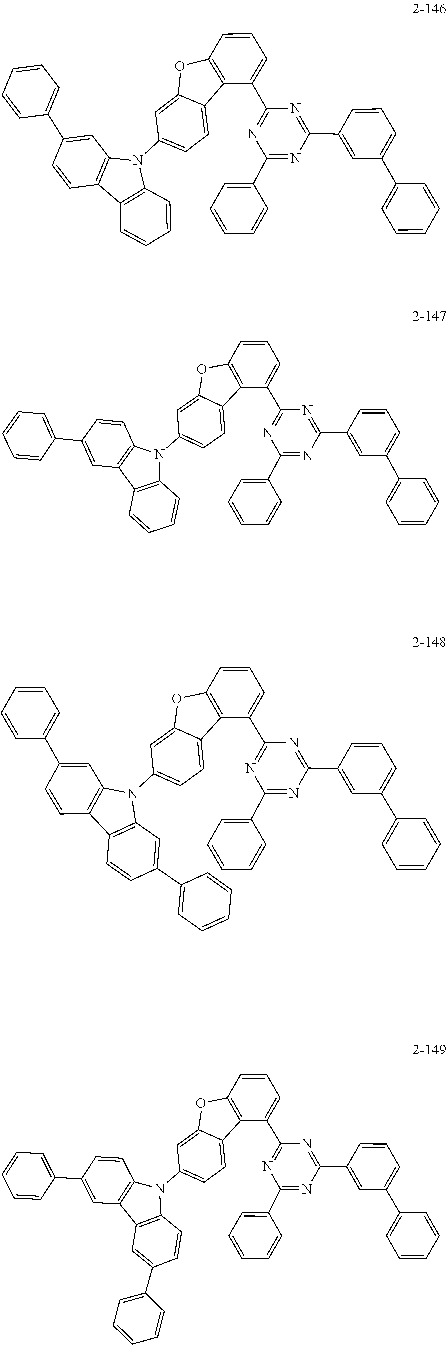 [SMILES: c1ccc(-c2cccc(-c3nc(-c4ccccc4)nc(-c4cccc5oc6cc(-n7c8ccccc8c8ccc(-c9ccccc9)cc87)ccc6c45)n3)c2)cc1 ; c1ccc(-c2cccc(-c3nc(-c4ccccc4)nc(-c4cccc5oc6cc(-n7c8ccccc8c8cc(-c9ccccc9)ccc87)ccc6c45)n3)c2)cc1 ; c1ccc(-c2cccc(-c3nc(-c4ccccc4)nc(-c4cccc5oc6cc(-n7c8cc(-c9ccccc9)ccc8c8ccc(-c9ccccc9)cc87)ccc6c45)n3)c2)cc1 ; c1ccc(-c2cccc(-c3nc(-c4ccccc4)nc(-c4cccc5oc6cc(-n7c8ccc(-c9ccccc9)cc8c8cc(-c9ccccc9)ccc87)ccc6c45)n3)c2)cc1]

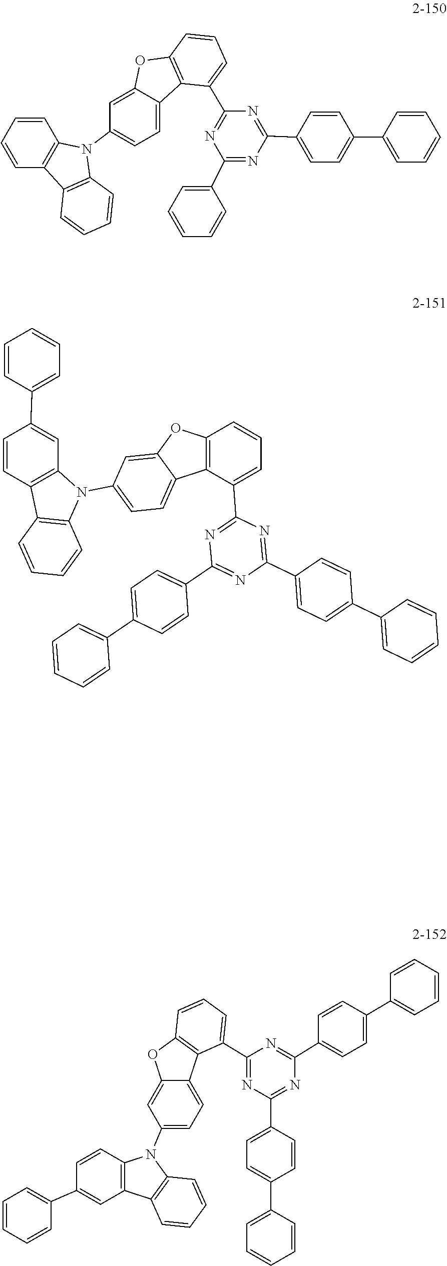 [SMILES: c1ccc(-c2ccc(-c3nc(-c4ccccc4)nc(-c4cccc5oc6cc(-n7c8ccccc8c8ccccc87)ccc6c45)n3)cc2)cc1 ; c1ccc(-c2ccc(-c3nc(-c4ccc(-c5ccccc5)cc4)nc(-c4cccc5oc6cc(-n7c8ccccc8c8ccc(-c9ccccc9)cc87)ccc6c45)n3)cc2)cc1 ; c1ccc(-c2ccc(-c3nc(-c4ccc(-c5ccccc5)cc4)nc(-c4cccc5oc6cc(-n7c8ccccc8c8cc(-c9ccccc9)ccc87)ccc6c45)n3)cc2)cc1]

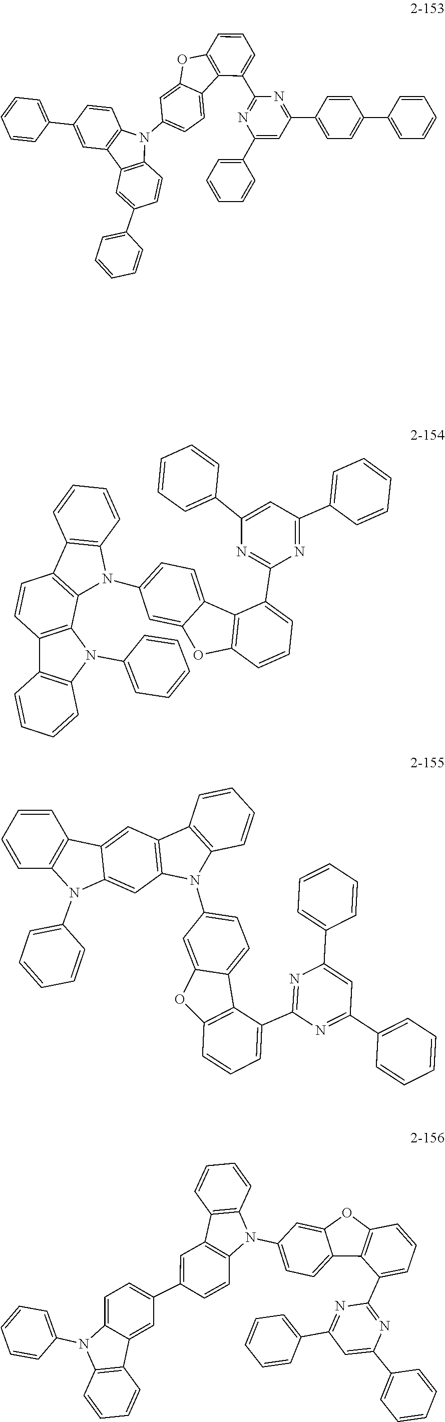 [SMILES: c1ccc(-c2ccc(-c3cc(-c4ccccc4)nc(-c4cccc5oc6cc(-n7c8ccc(-c9ccccc9)cc8c8cc(-c9ccccc9)ccc87)ccc6c45)n3)cc2)cc1 ; c1ccc(-c2cc(-c3ccccc3)nc(-c3cccc4oc5cc(-n6c7ccccc7c7ccc8c9ccccc9n(-c9ccccc9)c8c76)ccc5c34)n2)cc1 ; c1ccc(-c2cc(-c3ccccc3)nc(-c3cccc4oc5cc(-n6c7ccccc7c7cc8c9ccccc9n(-c9ccccc9)c8cc76)ccc5c34)n2)cc1 ; c1ccc(-c2cc(-c3ccccc3)nc(-c3cccc4oc5cc(-n6c7ccccc7c7cc(-c8ccc9c(c8)c8ccccc8n9-c8ccccc8)ccc76)ccc5c34)n2)cc1]

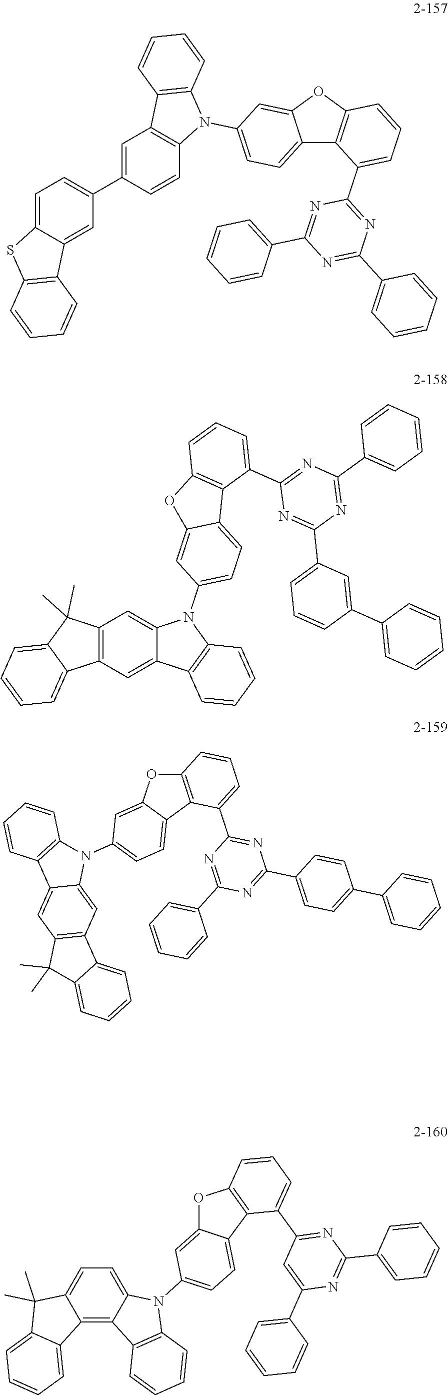 [SMILES: c1ccc(-c2nc(-c3ccccc3)nc(-c3cccc4oc5cc(-n6c7ccccc7c7cc(-c8ccc9sc%10ccccc%10c9c8)ccc76)ccc5c34)n2)cc1 ; CC1(C)c2ccccc2-c2cc3c4ccccc4n(-c4ccc5c(c4)oc4cccc(-c6nc(-c7ccccc7)nc(-c7cccc(-c8ccccc8)c7)n6)c45)c3cc21 ; CC1(C)c2ccccc2-c2cc3c(cc21)c1ccccc1n3-c1ccc2c(c1)oc1cccc(-c3nc(-c4ccccc4)nc(-c4ccc(-c5ccccc5)cc4)n3)c12 ; CC1(C)c2ccccc2-c2c1ccc1c2c2ccccc2n1-c1ccc2c(c1)oc1cccc(-c3cc(-c4ccccc4)nc(-c4ccccc4)n3)c12]

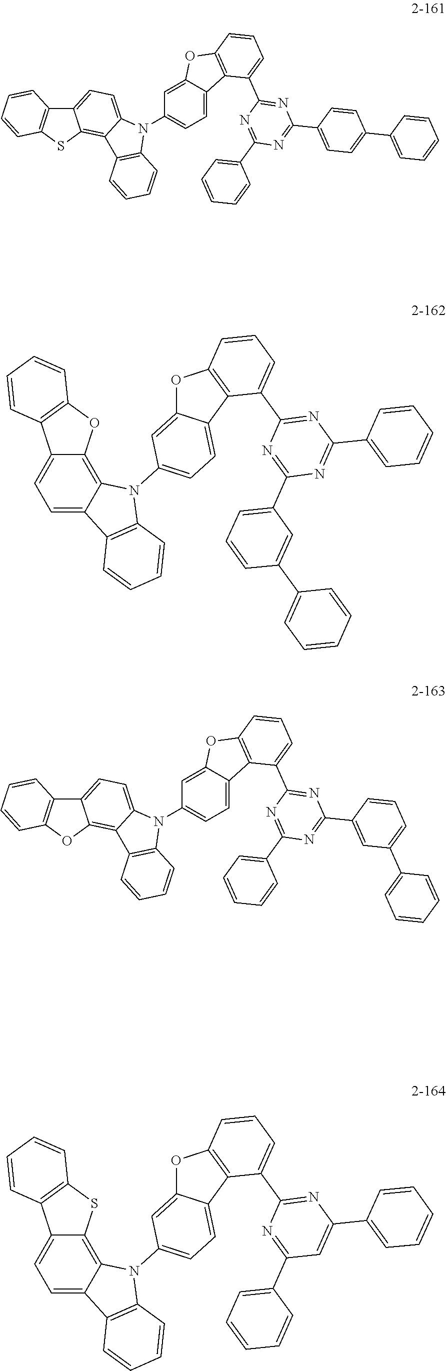 [SMILES: c1ccc(-c2ccc(-c3nc(-c4ccccc4)nc(-c4cccc5oc6cc(-n7c8ccccc8c8c9sc%10ccccc%10c9ccc87)ccc6c45)n3)cc2)cc1 ; c1ccc(-c2cccc(-c3nc(-c4ccccc4)nc(-c4cccc5oc6cc(-n7c8ccccc8c8ccc9c%10ccccc%10oc9c87)ccc6c45)n3)c2)cc1 ; c1ccc(-c2cccc(-c3nc(-c4ccccc4)nc(-c4cccc5oc6cc(-n7c8ccccc8c8c9oc%10ccccc%10c9ccc87)ccc6c45)n3)c2)cc1 ; c1ccc(-c2cc(-c3ccccc3)nc(-c3cccc4oc5cc(-n6c7ccccc7c7ccc8c9ccccc9sc8c76)ccc5c34)n2)cc1]

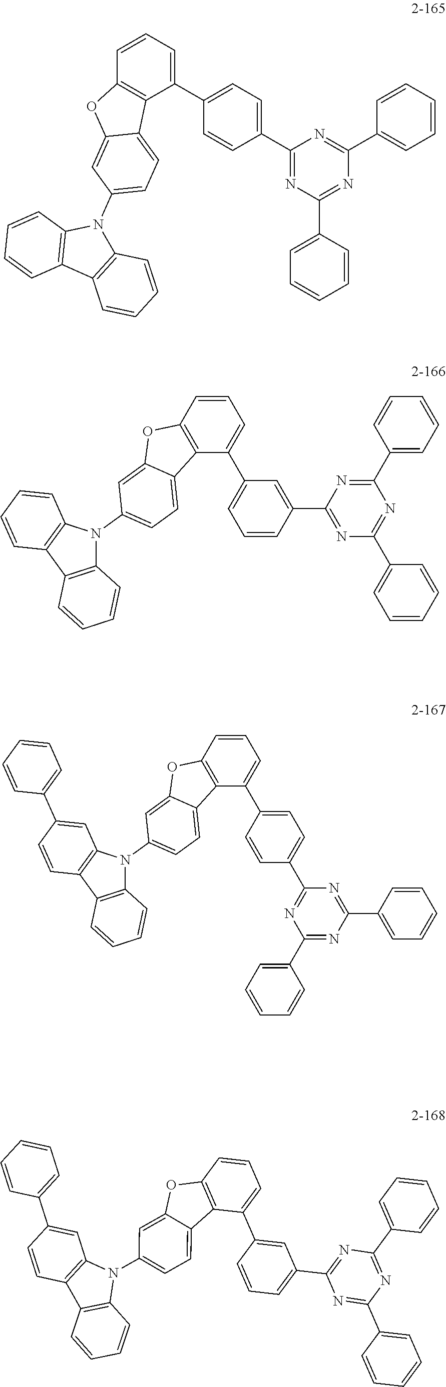 [SMILES: c1ccc(-c2nc(-c3ccccc3)nc(-c3ccc(-c4cccc5oc6cc(-n7c8ccccc8c8ccccc87)ccc6c45)cc3)n2)cc1 ; c1ccc(-c2nc(-c3ccccc3)nc(-c3cccc(-c4cccc5oc6cc(-n7c8ccccc8c8ccccc87)ccc6c45)c3)n2)cc1 ; c1ccc(-c2ccc3c4ccccc4n(-c4ccc5c(c4)oc4cccc(-c6ccc(-c7nc(-c8ccccc8)nc(-c8ccccc8)n7)cc6)c45)c3c2)cc1 ; c1ccc(-c2ccc3c4ccccc4n(-c4ccc5c(c4)oc4cccc(-c6cccc(-c7nc(-c8ccccc8)nc(-c8ccccc8)n7)c6)c45)c3c2)cc1]

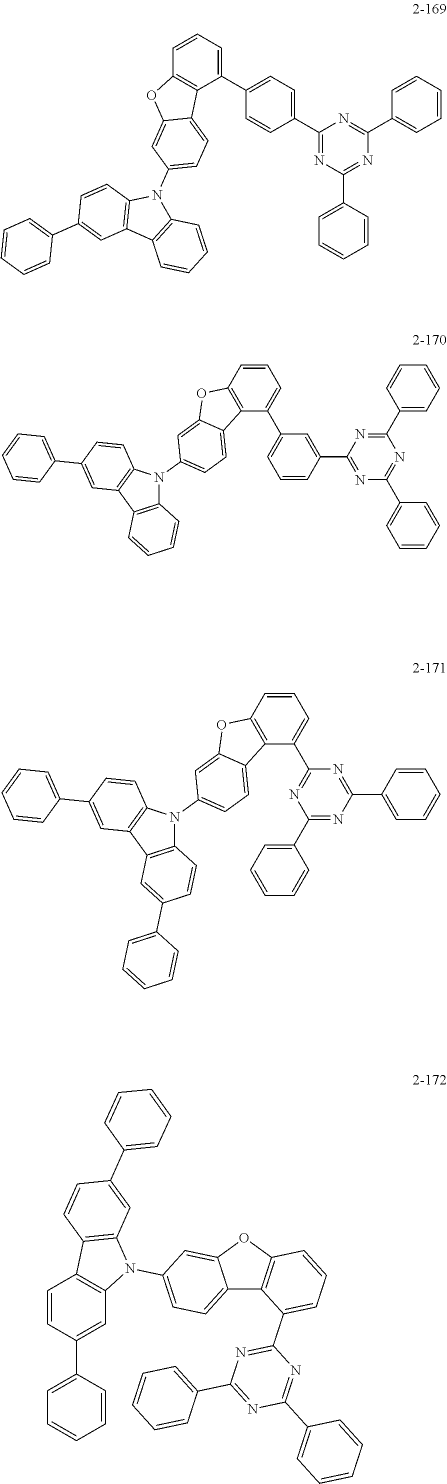 [SMILES: c1ccc(-c2ccc3c(c2)c2ccccc2n3-c2ccc3c(c2)oc2cccc(-c4ccc(-c5nc(-c6ccccc6)nc(-c6ccccc6)n5)cc4)c23)cc1 ; c1ccc(-c2ccc3c(c2)c2ccccc2n3-c2ccc3c(c2)oc2cccc(-c4cccc(-c5nc(-c6ccccc6)nc(-c6ccccc6)n5)c4)c23)cc1 ; c1ccc(-c2ccc3c(c2)c2cc(-c4ccccc4)ccc2n3-c2ccc3c(c2)oc2cccc(-c4nc(-c5ccccc5)nc(-c5ccccc5)n4)c23)cc1 ; c1ccc(-c2ccc3c4ccc(-c5ccccc5)cc4n(-c4ccc5c(c4)oc4cccc(-c6nc(-c7ccccc7)nc(-c7ccccc7)n6)c45)c3c2)cc1]

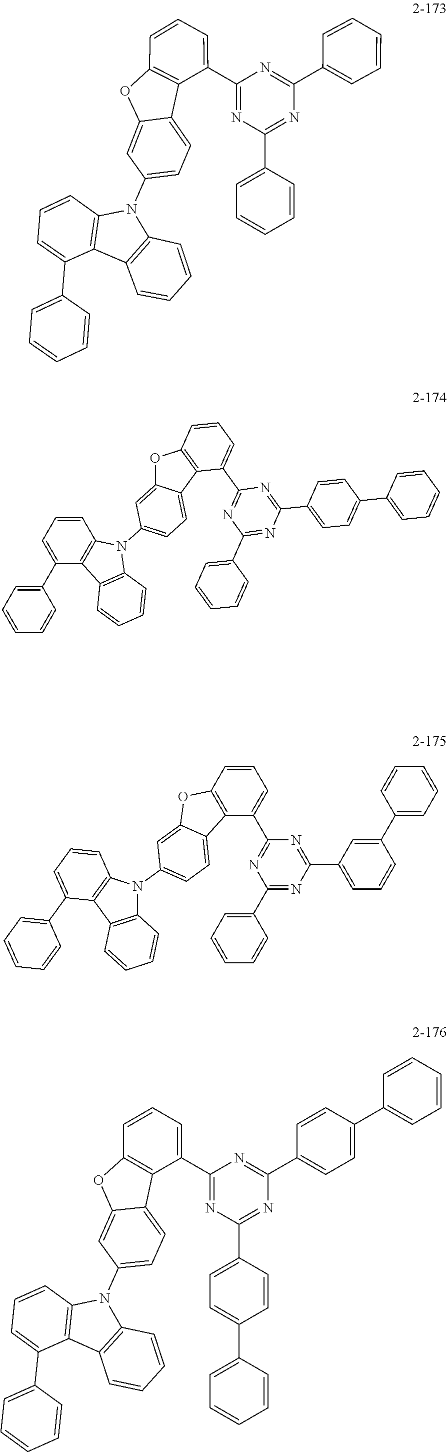 [SMILES: c1ccc(-c2nc(-c3ccccc3)nc(-c3cccc4oc5cc(-n6c7ccccc7c7c(-c8ccccc8)cccc76)ccc5c34)n2)cc1 ; c1ccc(-c2ccc(-c3nc(-c4ccccc4)nc(-c4cccc5oc6cc(-n7c8ccccc8c8c(-c9ccccc9)cccc87)ccc6c45)n3)cc2)cc1 ; c1ccc(-c2cccc(-c3nc(-c4ccccc4)nc(-c4cccc5oc6cc(-n7c8ccccc8c8c(-c9ccccc9)cccc87)ccc6c45)n3)c2)cc1 ; c1ccc(-c2ccc(-c3nc(-c4ccc(-c5ccccc5)cc4)nc(-c4cccc5oc6cc(-n7c8ccccc8c8c(-c9ccccc9)cccc87)ccc6c45)n3)cc2)cc1]

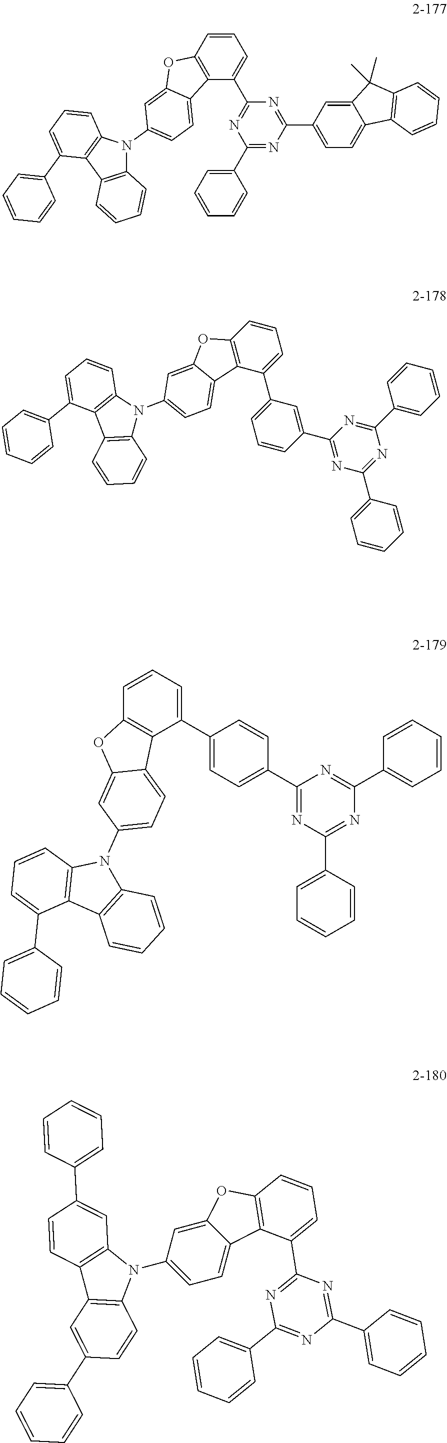 [SMILES: CC1(C)c2ccccc2-c2ccc(-c3nc(-c4ccccc4)nc(-c4cccc5oc6cc(-n7c8ccccc8c8c(-c9ccccc9)cccc87)ccc6c45)n3)cc21 ; c1ccc(-c2nc(-c3ccccc3)nc(-c3cccc(-c4cccc5oc6cc(-n7c8ccccc8c8c(-c9ccccc9)cccc87)ccc6c45)c3)n2)cc1 ; c1ccc(-c2nc(-c3ccccc3)nc(-c3ccc(-c4cccc5oc6cc(-n7c8ccccc8c8c(-c9ccccc9)cccc87)ccc6c45)cc3)n2)cc1 ; c1ccc(-c2ccc3c(c2)c2ccc(-c4ccccc4)cc2n3-c2ccc3c(c2)oc2cccc(-c4nc(-c5ccccc5)nc(-c5ccccc5)n4)c23)cc1]

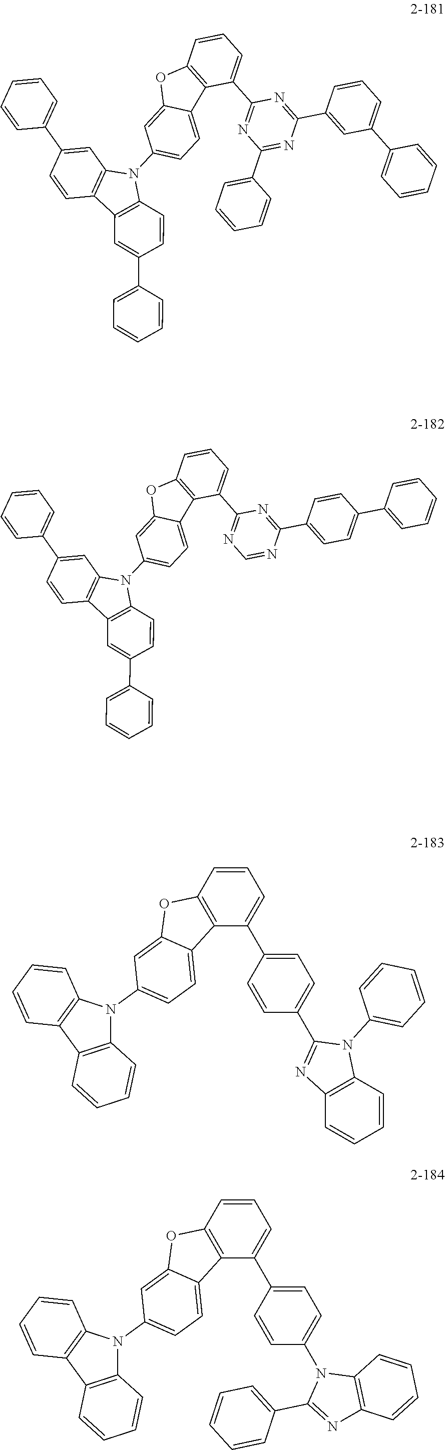 [SMILES: c1ccc(-c2cccc(-c3nc(-c4ccccc4)nc(-c4cccc5oc6cc(-n7c8ccc(-c9ccccc9)cc8c8ccc(-c9ccccc9)cc87)ccc6c45)n3)c2)cc1 ; c1ccc(-c2ccc(-c3ncnc(-c4cccc5oc6cc(-n7c8ccc(-c9ccccc9)cc8c8ccc(-c9ccccc9)cc87)ccc6c45)n3)cc2)cc1 ; c1ccc(-n2c(-c3ccc(-c4cccc5oc6cc(-n7c8ccccc8c8ccccc87)ccc6c45)cc3)nc3ccccc32)cc1 ; c1ccc(-c2nc3ccccc3n2-c2ccc(-c3cccc4oc5cc(-n6c7ccccc7c7ccccc76)ccc5c34)cc2)cc1]

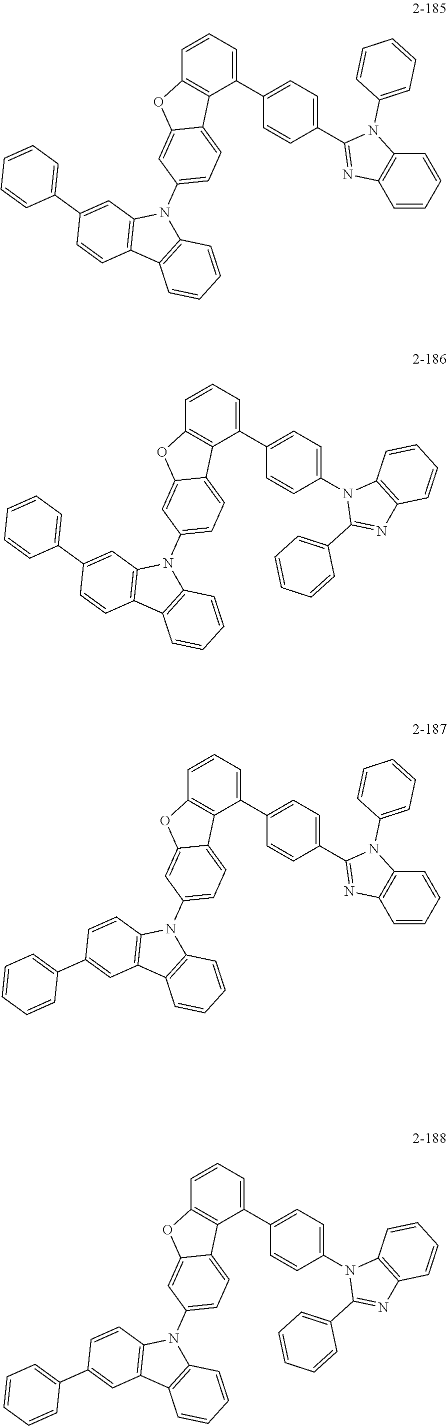 [SMILES: c1ccc(-c2ccc3c4ccccc4n(-c4ccc5c(c4)oc4cccc(-c6ccc(-c7nc8ccccc8n7-c7ccccc7)cc6)c45)c3c2)cc1 ; c1ccc(-c2ccc3c4ccccc4n(-c4ccc5c(c4)oc4cccc(-c6ccc(-n7c(-c8ccccc8)nc8ccccc87)cc6)c45)c3c2)cc1 ; c1ccc(-c2ccc3c(c2)c2ccccc2n3-c2ccc3c(c2)oc2cccc(-c4ccc(-c5nc6ccccc6n5-c5ccccc5)cc4)c23)cc1 ; c1ccc(-c2ccc3c(c2)c2ccccc2n3-c2ccc3c(c2)oc2cccc(-c4ccc(-n5c(-c6ccccc6)nc6ccccc65)cc4)c23)cc1]

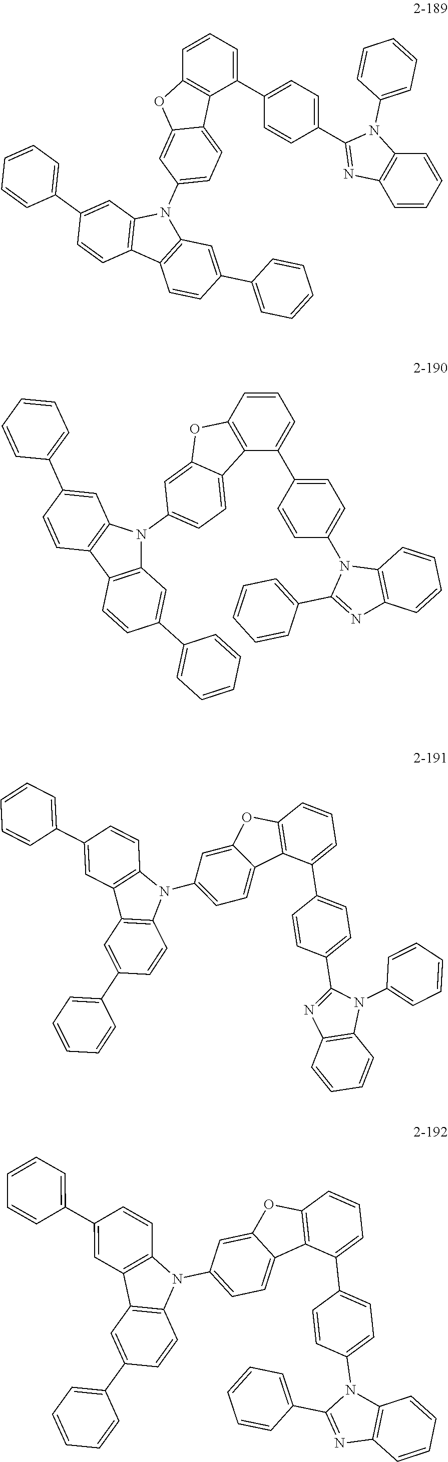 [SMILES: c1ccc(-c2ccc3c4ccc(-c5ccccc5)cc4n(-c4ccc5c(c4)oc4cccc(-c6ccc(-c7nc8ccccc8n7-c7ccccc7)cc6)c45)c3c2)cc1 ; c1ccc(-c2ccc3c4ccc(-c5ccccc5)cc4n(-c4ccc5c(c4)oc4cccc(-c6ccc(-n7c(-c8ccccc8)nc8ccccc87)cc6)c45)c3c2)cc1 ; c1ccc(-c2ccc3c(c2)c2cc(-c4ccccc4)ccc2n3-c2ccc3c(c2)oc2cccc(-c4ccc(-c5nc6ccccc6n5-c5ccccc5)cc4)c23)cc1 ; c1ccc(-c2ccc3c(c2)c2cc(-c4ccccc4)ccc2n3-c2ccc3c(c2)oc2cccc(-c4ccc(-n5c(-c6ccccc6)nc6ccccc65)cc4)c23)cc1]

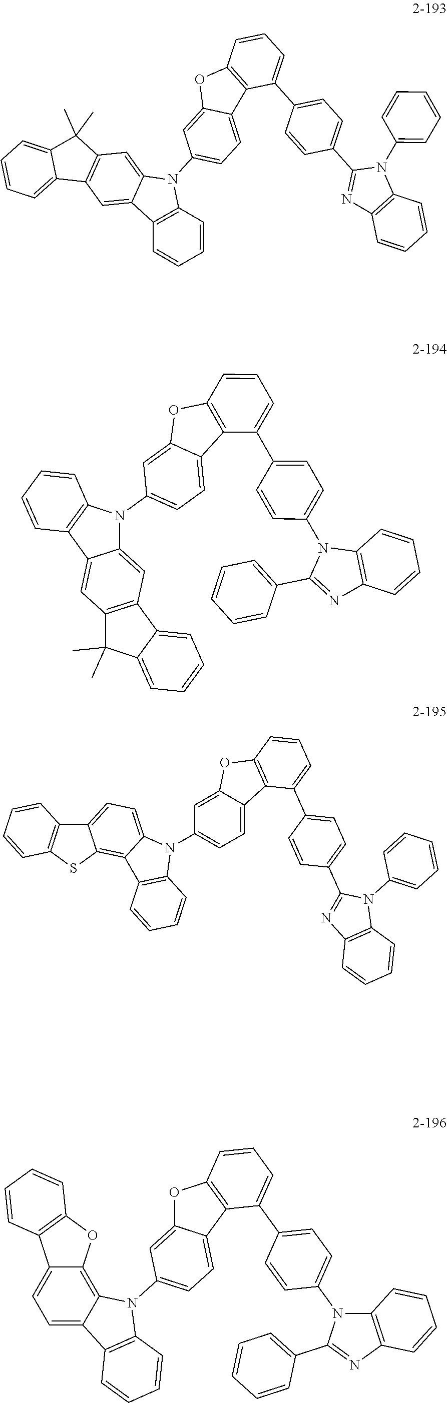 [SMILES: CC1(C)c2ccccc2-c2cc3c4ccccc4n(-c4ccc5c(c4)oc4cccc(-c6ccc(-c7nc8ccccc8n7-c7ccccc7)cc6)c45)c3cc21 ; CC1(C)c2ccccc2-c2cc3c(cc21)c1ccccc1n3-c1ccc2c(c1)oc1cccc(-c3ccc(-n4c(-c5ccccc5)nc5ccccc54)cc3)c12 ; c1ccc(-n2c(-c3ccc(-c4cccc5oc6cc(-n7c8ccccc8c8c9sc%10ccccc%10c9ccc87)ccc6c45)cc3)nc3ccccc32)cc1 ; c1ccc(-c2nc3ccccc3n2-c2ccc(-c3cccc4oc5cc(-n6c7ccccc7c7ccc8c9ccccc9oc8c76)ccc5c34)cc2)cc1]

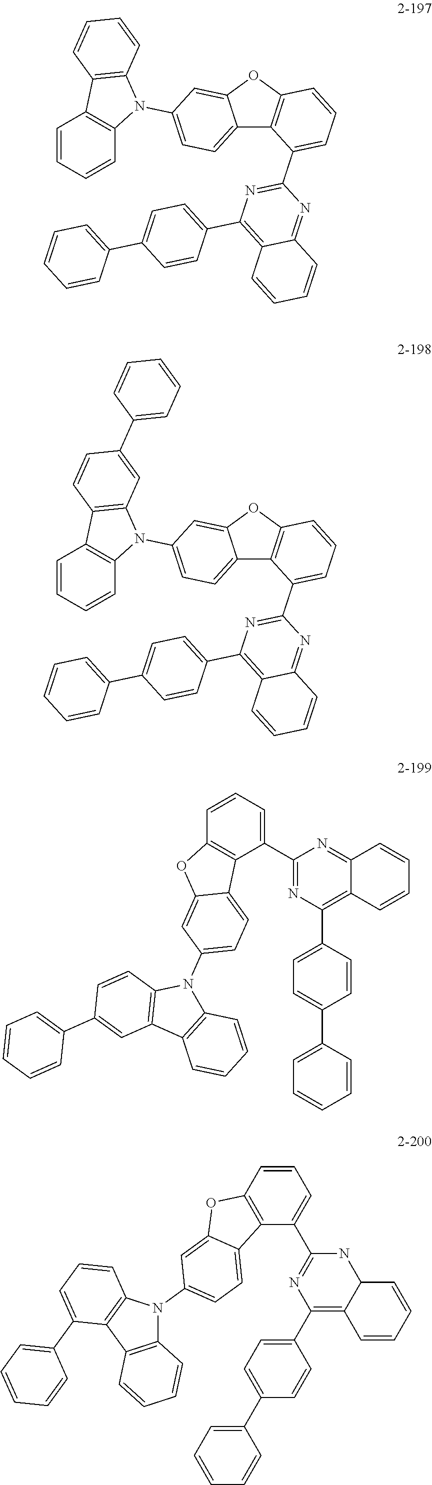

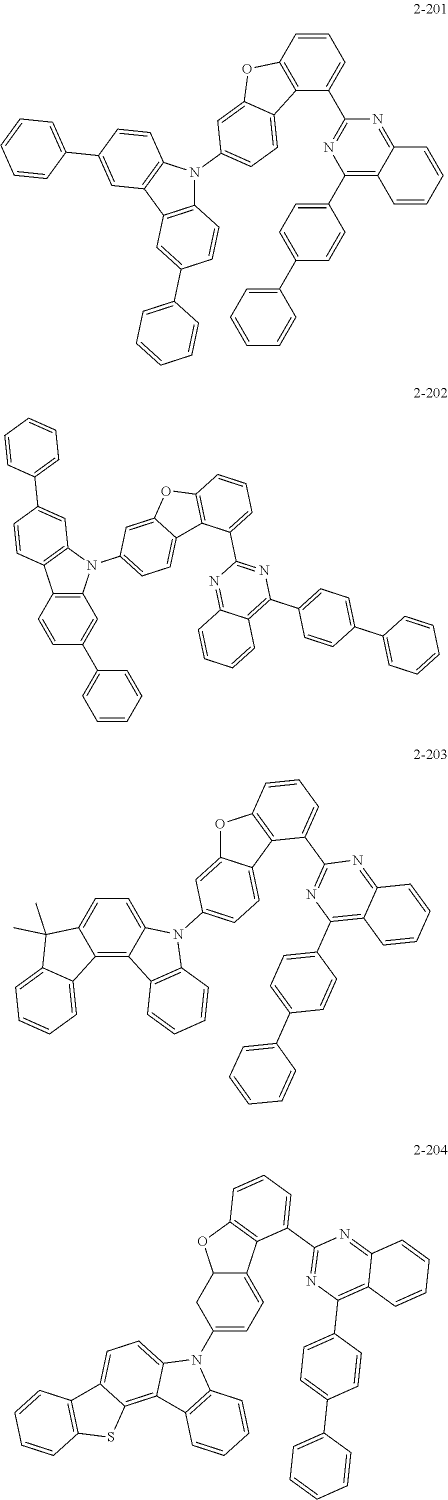 [SMILES: c1ccc(-c2ccc(-c3nc(-c4cccc5oc6cc(-n7c8ccc(-c9ccccc9)cc8c8cc(-c9ccccc9)ccc87)ccc6c45)nc4ccccc34)cc2)cc1 ; c1ccc(-c2ccc(-c3nc(-c4cccc5oc6cc(-n7c8cc(-c9ccccc9)ccc8c8ccc(-c9ccccc9)cc87)ccc6c45)nc4ccccc34)cc2)cc1 ; CC1(C)c2ccccc2-c2c1ccc1c2c2ccccc2n1-c1ccc2c(c1)oc1cccc(-c3nc(-c4ccc(-c5ccccc5)cc4)c4ccccc4n3)c12 ; C1=C2c3c(cccc3-c3nc(-c4ccc(-c5ccccc5)cc4)c4ccccc4n3)OC2CC(n2c3ccccc3c3c4sc5ccccc5c4ccc32)=C1]

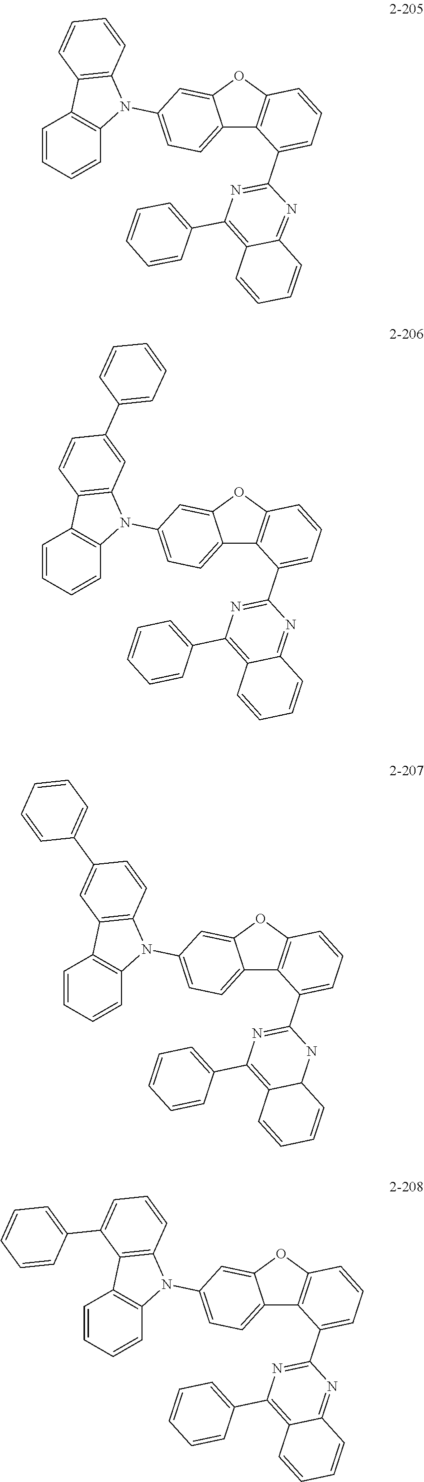

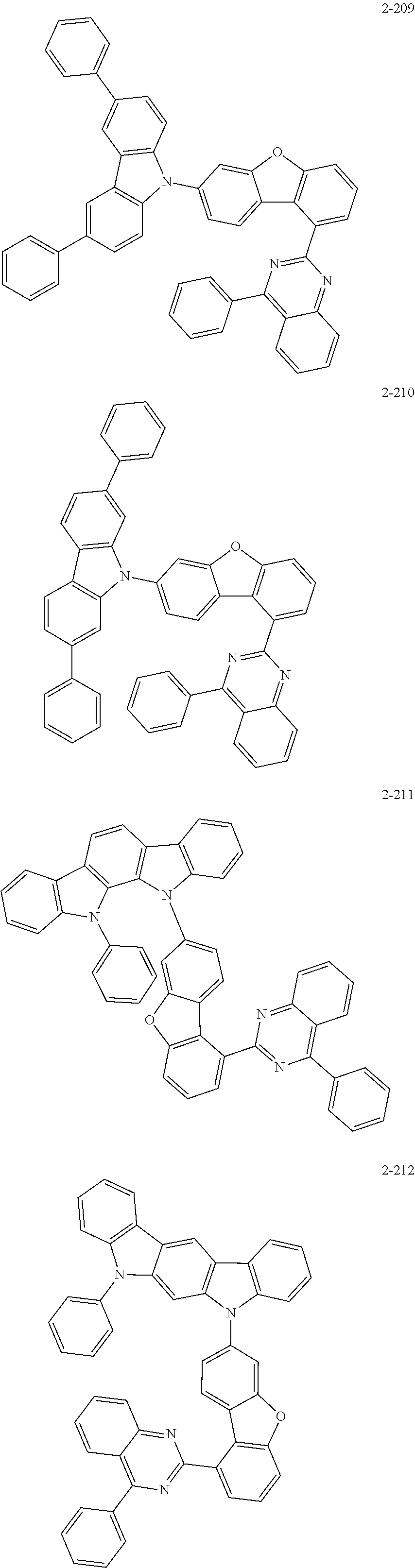 [SMILES: c1ccc(-c2ccc3c(c2)c2cc(-c4ccccc4)ccc2n3-c2ccc3c(c2)oc2cccc(-c4nc(-c5ccccc5)c5ccccc5n4)c23)cc1 ; c1ccc(-c2ccc3c4ccc(-c5ccccc5)cc4n(-c4ccc5c(c4)oc4cccc(-c6nc(-c7ccccc7)c7ccccc7n6)c45)c3c2)cc1 ; c1ccc(-c2nc(-c3cccc4oc5cc(-n6c7ccccc7c7ccc8c9ccccc9n(-c9ccccc9)c8c76)ccc5c34)nc3ccccc23)cc1 ; c1ccc(-c2nc(-c3cccc4oc5cc(-n6c7ccccc7c7cc8c9ccccc9n(-c9ccccc9)c8cc76)ccc5c34)nc3ccccc23)cc1]

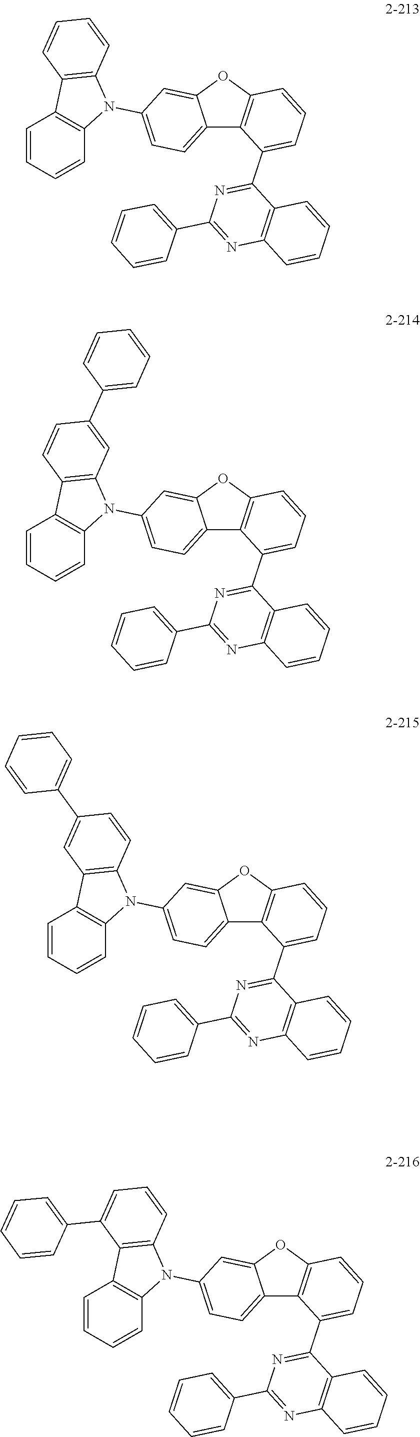 [SMILES: c1ccc(-c2nc(-c3cccc4oc5cc(-n6c7ccccc7c7ccccc76)ccc5c34)c3ccccc3n2)cc1 ; c1ccc(-c2ccc3c4ccccc4n(-c4ccc5c(c4)oc4cccc(-c6nc(-c7ccccc7)nc7ccccc67)c45)c3c2)cc1 ; c1ccc(-c2ccc3c(c2)c2ccccc2n3-c2ccc3c(c2)oc2cccc(-c4nc(-c5ccccc5)nc5ccccc45)c23)cc1 ; c1ccc(-c2nc(-c3cccc4oc5cc(-n6c7ccccc7c7c(-c8ccccc8)cccc76)ccc5c34)c3ccccc3n2)cc1]

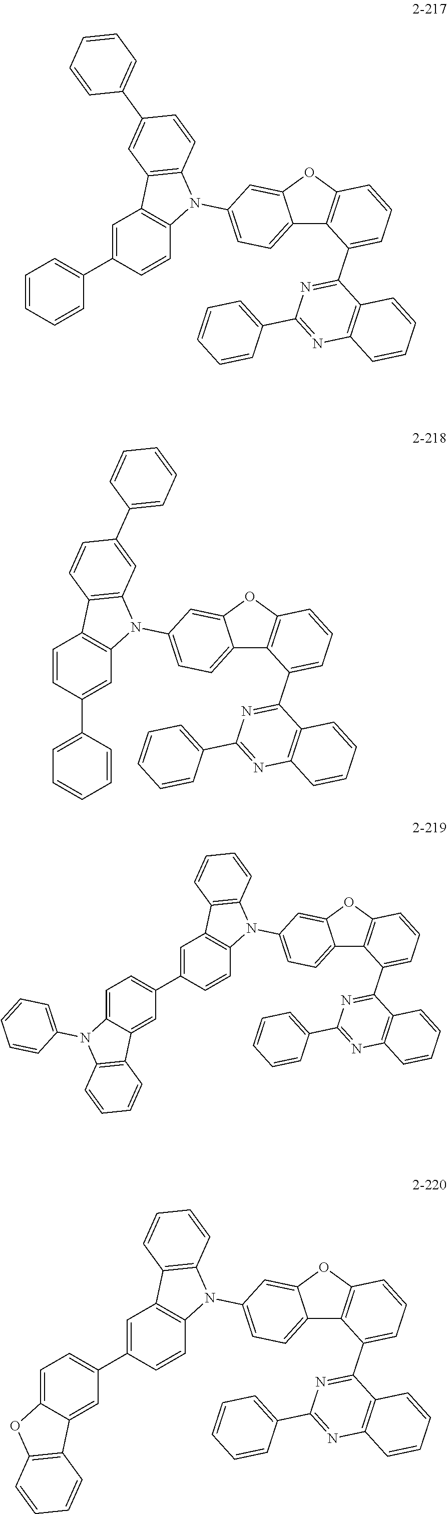 [SMILES: c1ccc(-c2ccc3c(c2)c2cc(-c4ccccc4)ccc2n3-c2ccc3c(c2)oc2cccc(-c4nc(-c5ccccc5)nc5ccccc45)c23)cc1 ; c1ccc(-c2ccc3c4ccc(-c5ccccc5)cc4n(-c4ccc5c(c4)oc4cccc(-c6nc(-c7ccccc7)nc7ccccc67)c45)c3c2)cc1 ; c1ccc(-c2nc(-c3cccc4oc5cc(-n6c7ccccc7c7cc(-c8ccc9c(c8)c8ccccc8n9-c8ccccc8)ccc76)ccc5c34)c3ccccc3n2)cc1 ; c1ccc(-c2nc(-c3cccc4oc5cc(-n6c7ccccc7c7cc(-c8ccc9oc%10ccccc%10c9c8)ccc76)ccc5c34)c3ccccc3n2)cc1]

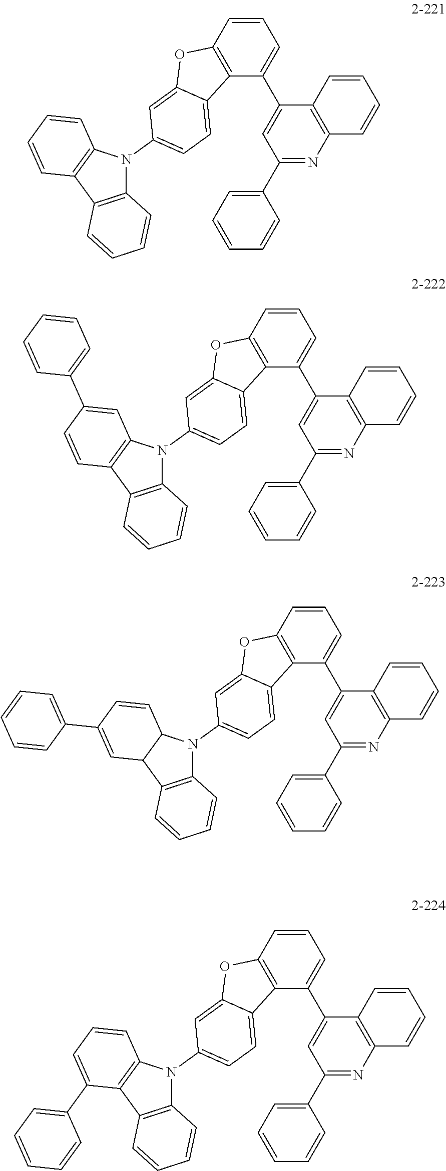 [SMILES: c1ccc(-c2cc(-c3cccc4oc5cc(-n6c7ccccc7c7ccccc76)ccc5c34)c3ccccc3n2)cc1 ; c1ccc(-c2ccc3c4ccccc4n(-c4ccc5c(c4)oc4cccc(-c6cc(-c7ccccc7)nc7ccccc67)c45)c3c2)cc1 ; C1=CC2C(C=C1c1ccccc1)c1ccccc1N2c1ccc2c(c1)oc1cccc(-c3cc(-c4ccccc4)nc4ccccc34)c12 ; c1ccc(-c2cc(-c3cccc4oc5cc(-n6c7ccccc7c7c(-c8ccccc8)cccc76)ccc5c34)c3ccccc3n2)cc1]

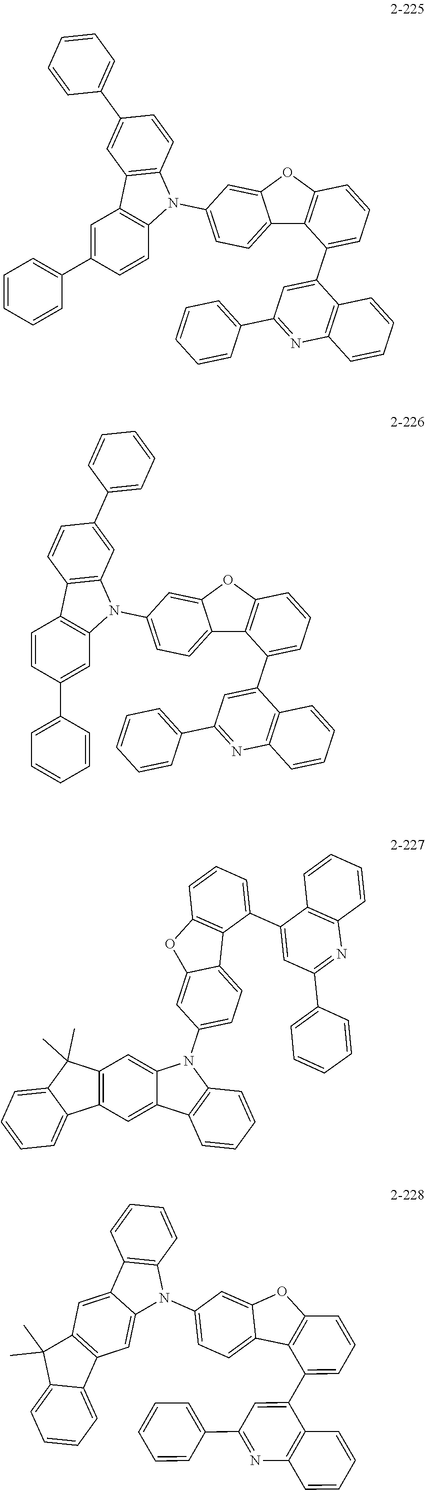 [SMILES: c1ccc(-c2ccc3c(c2)c2cc(-c4ccccc4)ccc2n3-c2ccc3c(c2)oc2cccc(-c4cc(-c5ccccc5)nc5ccccc45)c23)cc1 ; c1ccc(-c2ccc3c4ccc(-c5ccccc5)cc4n(-c4ccc5c(c4)oc4cccc(-c6cc(-c7ccccc7)nc7ccccc67)c45)c3c2)cc1 ; CC1(C)c2ccccc2-c2cc3c4ccccc4n(-c4ccc5c(c4)oc4cccc(-c6cc(-c7ccccc7)nc7ccccc67)c45)c3cc21 ; CC1(C)c2ccccc2-c2cc3c(cc21)c1ccccc1n3-c1ccc2c(c1)oc1cccc(-c3cc(-c4ccccc4)nc4ccccc34)c12]

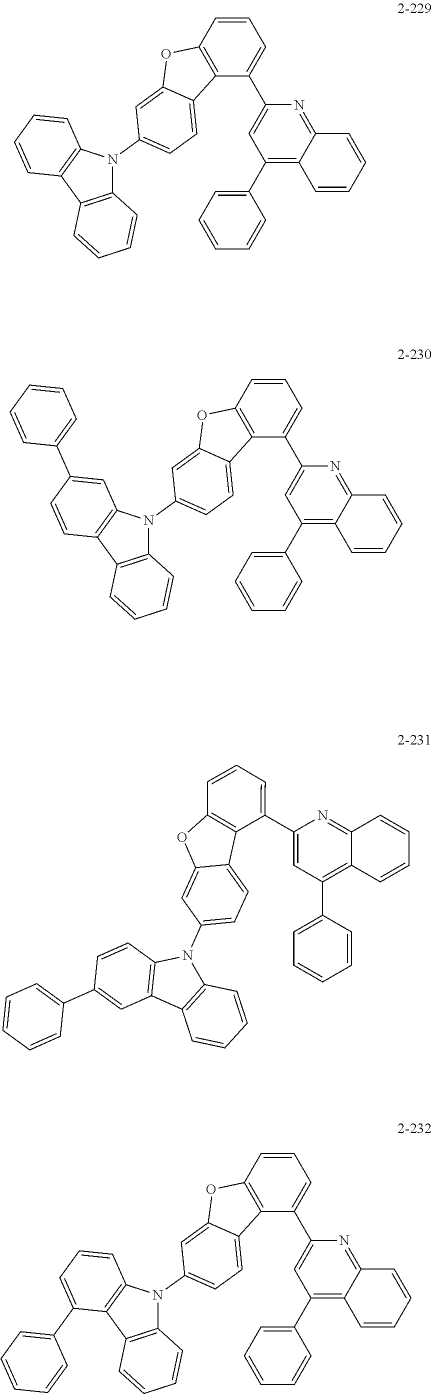 [SMILES: c1ccc(-c2cc(-c3cccc4oc5cc(-n6c7ccccc7c7ccccc76)ccc5c34)nc3ccccc23)cc1 ; c1ccc(-c2ccc3c4ccccc4n(-c4ccc5c(c4)oc4cccc(-c6cc(-c7ccccc7)c7ccccc7n6)c45)c3c2)cc1 ; c1ccc(-c2ccc3c(c2)c2ccccc2n3-c2ccc3c(c2)oc2cccc(-c4cc(-c5ccccc5)c5ccccc5n4)c23)cc1 ; c1ccc(-c2cc(-c3cccc4oc5cc(-n6c7ccccc7c7c(-c8ccccc8)cccc76)ccc5c34)nc3ccccc23)cc1]

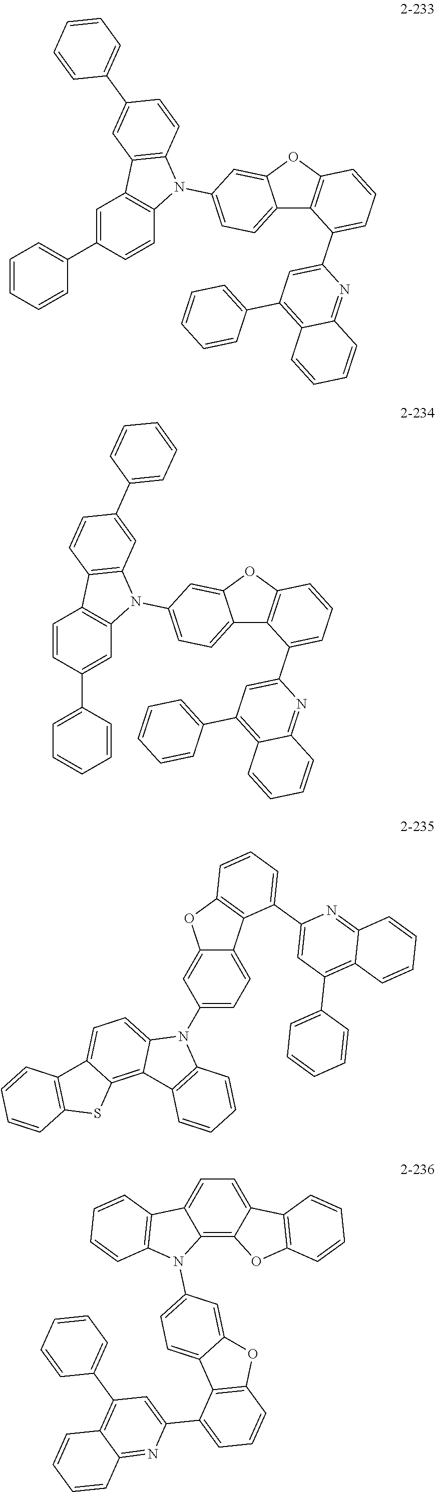 [SMILES: c1ccc(-c2ccc3c(c2)c2cc(-c4ccccc4)ccc2n3-c2ccc3c(c2)oc2cccc(-c4cc(-c5ccccc5)c5ccccc5n4)c23)cc1 ; c1ccc(-c2ccc3c4ccc(-c5ccccc5)cc4n(-c4ccc5c(c4)oc4cccc(-c6cc(-c7ccccc7)c7ccccc7n6)c45)c3c2)cc1 ; c1ccc(-c2cc(-c3cccc4oc5cc(-n6c7ccccc7c7c8sc9ccccc9c8ccc76)ccc5c34)nc3ccccc23)cc1 ; c1ccc(-c2cc(-c3cccc4oc5cc(-n6c7ccccc7c7ccc8c9ccccc9oc8c76)ccc5c34)nc3ccccc23)cc1]

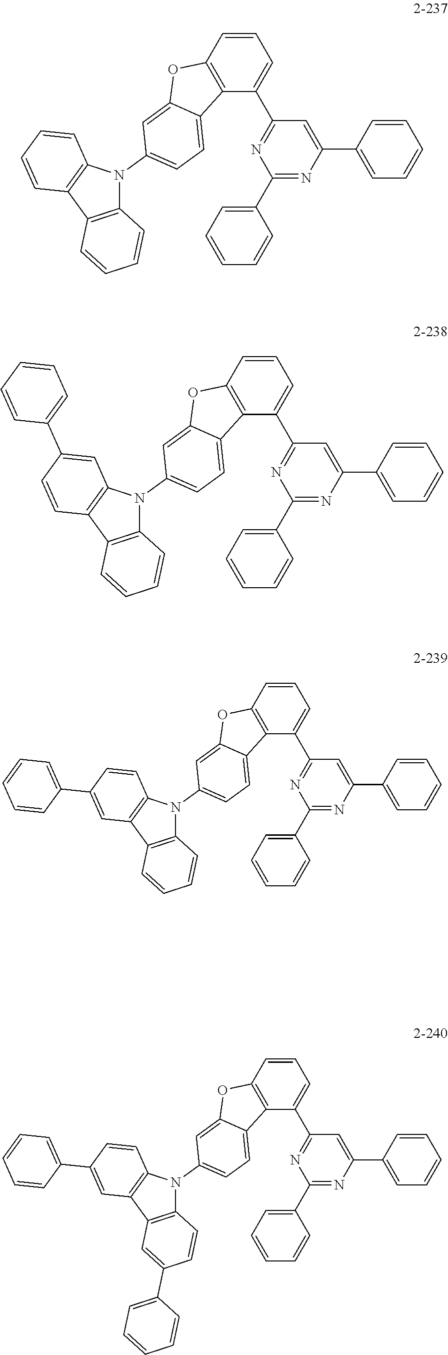 [SMILES: c1ccc(-c2cc(-c3cccc4oc5cc(-n6c7ccccc7c7ccccc76)ccc5c34)nc(-c3ccccc3)n2)cc1 ; c1ccc(-c2ccc3c4ccccc4n(-c4ccc5c(c4)oc4cccc(-c6cc(-c7ccccc7)nc(-c7ccccc7)n6)c45)c3c2)cc1 ; c1ccc(-c2ccc3c(c2)c2ccccc2n3-c2ccc3c(c2)oc2cccc(-c4cc(-c5ccccc5)nc(-c5ccccc5)n4)c23)cc1 ; c1ccc(-c2ccc3c(c2)c2cc(-c4ccccc4)ccc2n3-c2ccc3c(c2)oc2cccc(-c4cc(-c5ccccc5)nc(-c5ccccc5)n4)c23)cc1]

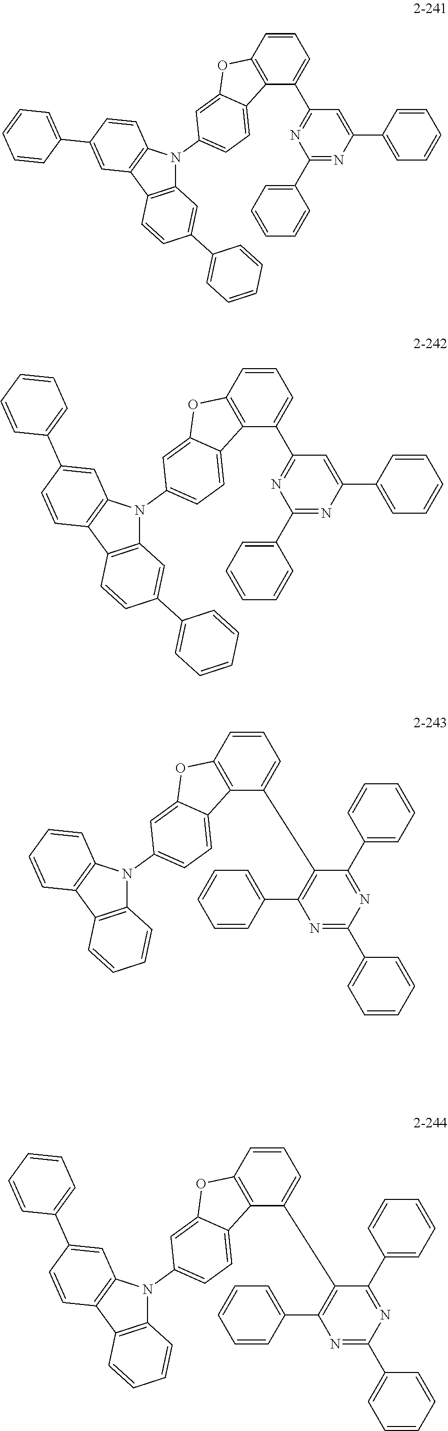 [SMILES: c1ccc(-c2ccc3c(c2)c2ccc(-c4ccccc4)cc2n3-c2ccc3c(c2)oc2cccc(-c4cc(-c5ccccc5)nc(-c5ccccc5)n4)c23)cc1 ; c1ccc(-c2ccc3c4ccc(-c5ccccc5)cc4n(-c4ccc5c(c4)oc4cccc(-c6cc(-c7ccccc7)nc(-c7ccccc7)n6)c45)c3c2)cc1 ; c1ccc(-c2nc(-c3ccccc3)c(-c3cccc4oc5cc(-n6c7ccccc7c7ccccc76)ccc5c34)c(-c3ccccc3)n2)cc1 ; c1ccc(-c2ccc3c4ccccc4n(-c4ccc5c(c4)oc4cccc(-c6c(-c7ccccc7)nc(-c7ccccc7)nc6-c6ccccc6)c45)c3c2)cc1]

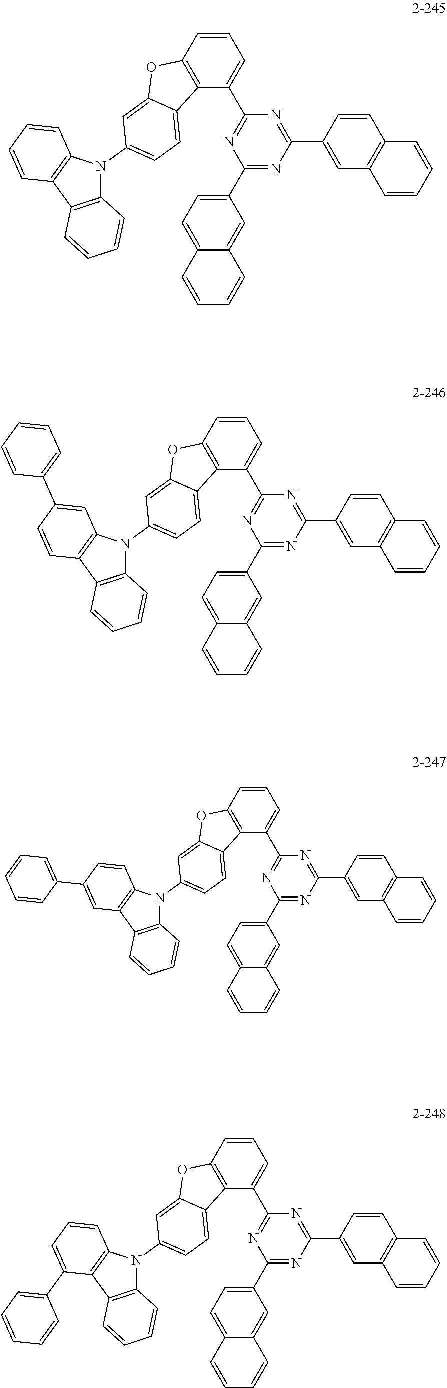 [SMILES: c1ccc2cc(-c3nc(-c4ccc5ccccc5c4)nc(-c4cccc5oc6cc(-n7c8ccccc8c8ccccc87)ccc6c45)n3)ccc2c1 ; c1ccc(-c2ccc3c4ccccc4n(-c4ccc5c(c4)oc4cccc(-c6nc(-c7ccc8ccccc8c7)nc(-c7ccc8ccccc8c7)n6)c45)c3c2)cc1 ; c1ccc(-c2ccc3c(c2)c2ccccc2n3-c2ccc3c(c2)oc2cccc(-c4nc(-c5ccc6ccccc6c5)nc(-c5ccc6ccccc6c5)n4)c23)cc1 ; c1ccc(-c2cccc3c2c2ccccc2n3-c2ccc3c(c2)oc2cccc(-c4nc(-c5ccc6ccccc6c5)nc(-c5ccc6ccccc6c5)n4)c23)cc1]

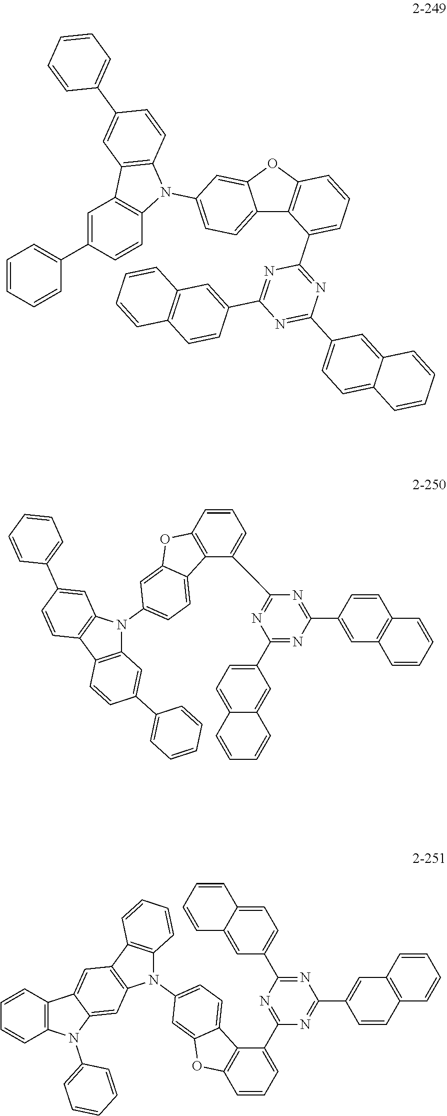 [SMILES: c1ccc(-c2ccc3c(c2)c2cc(-c4ccccc4)ccc2n3-c2ccc3c(c2)oc2cccc(-c4nc(-c5ccc6ccccc6c5)nc(-c5ccc6ccccc6c5)n4)c23)cc1 ; c1ccc(-c2ccc3c4ccc(-c5ccccc5)cc4n(-c4ccc5c(c4)oc4cccc(-c6nc(-c7ccc8ccccc8c7)nc(-c7ccc8ccccc8c7)n6)c45)c3c2)cc1 ; c1ccc(-n2c3ccccc3c3cc4c5ccccc5n(-c5ccc6c(c5)oc5cccc(-c7nc(-c8ccc9ccccc9c8)nc(-c8ccc9ccccc9c8)n7)c56)c4cc32)cc1]

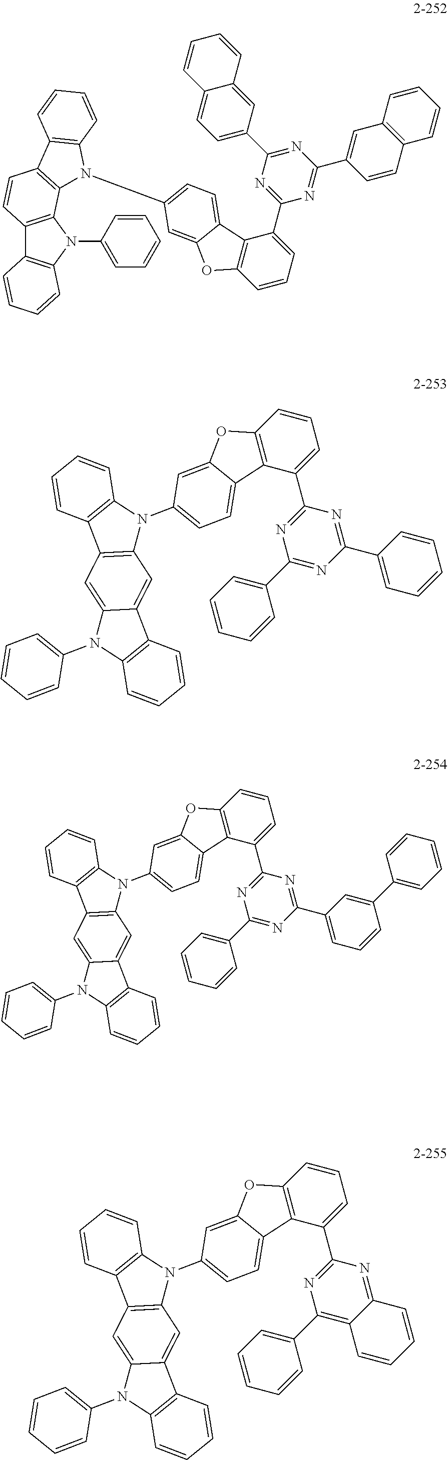 [SMILES: c1ccc(-n2c3ccccc3c3ccc4c5ccccc5n(-c5ccc6c(c5)oc5cccc(-c7nc(-c8ccc9ccccc9c8)nc(-c8ccc9ccccc9c8)n7)c56)c4c32)cc1 ; c1ccc(-c2nc(-c3ccccc3)nc(-c3cccc4oc5cc(-n6c7ccccc7c7cc8c(cc76)c6ccccc6n8-c6ccccc6)ccc5c34)n2)cc1 ; c1ccc(-c2cccc(-c3nc(-c4ccccc4)nc(-c4cccc5oc6cc(-n7c8ccccc8c8cc9c(cc87)c7ccccc7n9-c7ccccc7)ccc6c45)n3)c2)cc1 ; c1ccc(-c2nc(-c3cccc4oc5cc(-n6c7ccccc7c7cc8c(cc76)c6ccccc6n8-c6ccccc6)ccc5c34)nc3ccccc23)cc1]

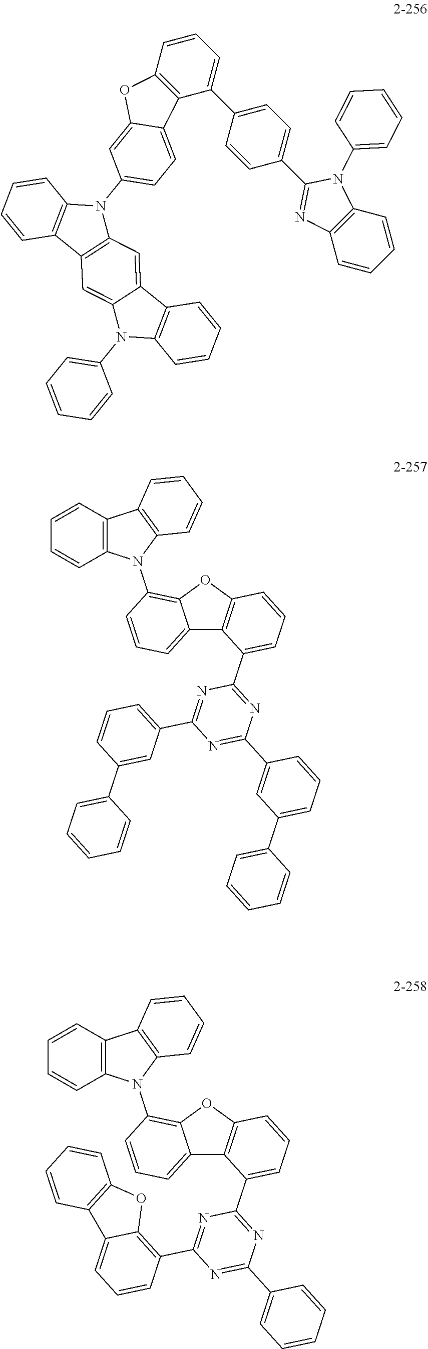 [SMILES: c1ccc(-n2c(-c3ccc(-c4cccc5oc6cc(-n7c8ccccc8c8cc9c(cc87)c7ccccc7n9-c7ccccc7)ccc6c45)cc3)nc3ccccc32)cc1 ; c1ccc(-c2cccc(-c3nc(-c4cccc(-c5ccccc5)c4)nc(-c4cccc5oc6c(-n7c8ccccc8c8ccccc87)cccc6c45)n3)c2)cc1 ; c1ccc(-c2nc(-c3cccc4c3oc3ccccc34)nc(-c3cccc4oc5c(-n6c7ccccc7c7ccccc76)cccc5c34)n2)cc1]

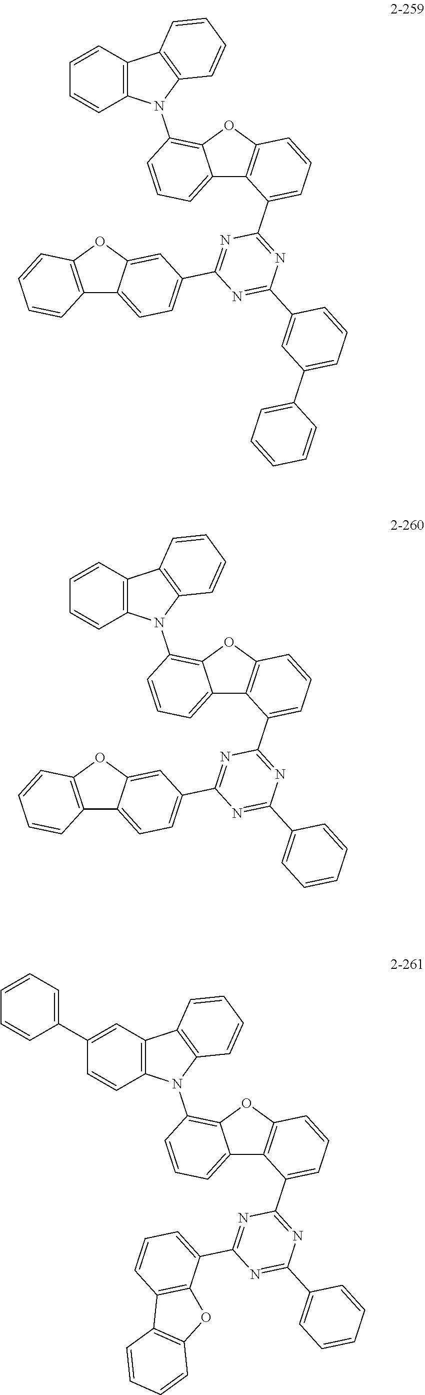 [SMILES: c1ccc(-c2cccc(-c3nc(-c4ccc5c(c4)oc4ccccc45)nc(-c4cccc5oc6c(-n7c8ccccc8c8ccccc87)cccc6c45)n3)c2)cc1 ; c1ccc(-c2nc(-c3ccc4c(c3)oc3ccccc34)nc(-c3cccc4oc5c(-n6c7ccccc7c7ccccc76)cccc5c34)n2)cc1 ; c1ccc(-c2ccc3c(c2)c2ccccc2n3-c2cccc3c2oc2cccc(-c4nc(-c5ccccc5)nc(-c5cccc6c5oc5ccccc56)n4)c23)cc1]

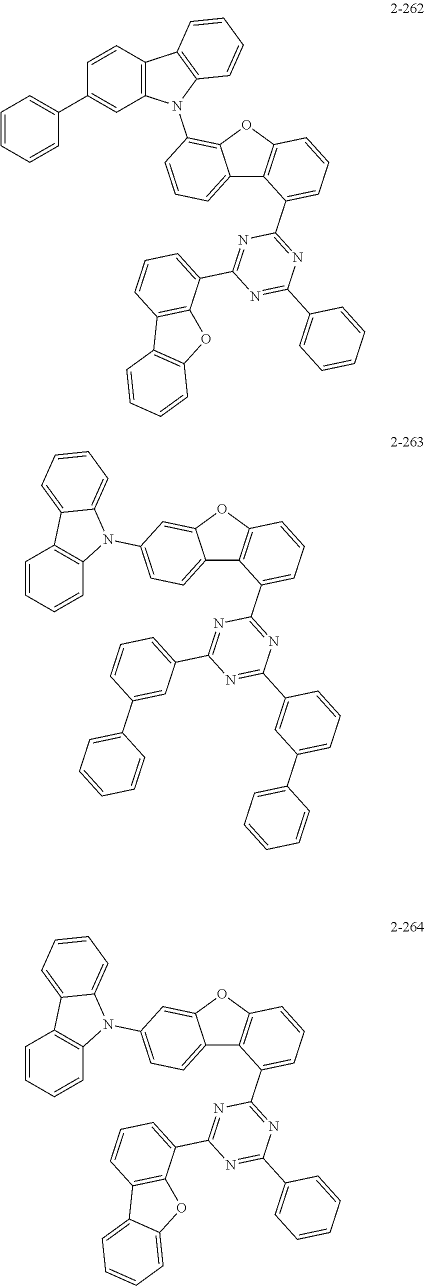 [SMILES: c1ccc(-c2ccc3c4ccccc4n(-c4cccc5c4oc4cccc(-c6nc(-c7ccccc7)nc(-c7cccc8c7oc7ccccc78)n6)c45)c3c2)cc1 ; c1ccc(-c2cccc(-c3nc(-c4cccc(-c5ccccc5)c4)nc(-c4cccc5oc6cc(-n7c8ccccc8c8ccccc87)ccc6c45)n3)c2)cc1 ; c1ccc(-c2nc(-c3cccc4c3oc3ccccc34)nc(-c3cccc4oc5cc(-n6c7ccccc7c7ccccc76)ccc5c34)n2)cc1]

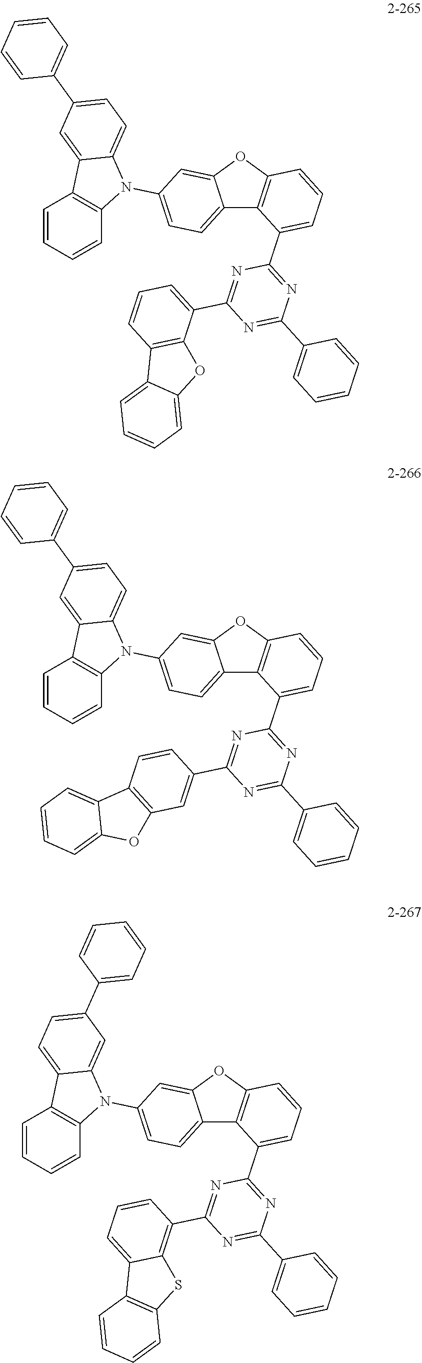 [SMILES: c1ccc(-c2ccc3c(c2)c2ccccc2n3-c2ccc3c(c2)oc2cccc(-c4nc(-c5ccccc5)nc(-c5cccc6c5oc5ccccc56)n4)c23)cc1 ; c1ccc(-c2ccc3c(c2)c2ccccc2n3-c2ccc3c(c2)oc2cccc(-c4nc(-c5ccccc5)nc(-c5ccc6c(c5)oc5ccccc56)n4)c23)cc1 ; c1ccc(-c2ccc3c4ccccc4n(-c4ccc5c(c4)oc4cccc(-c6nc(-c7ccccc7)nc(-c7cccc8c7sc7ccccc78)n6)c45)c3c2)cc1]

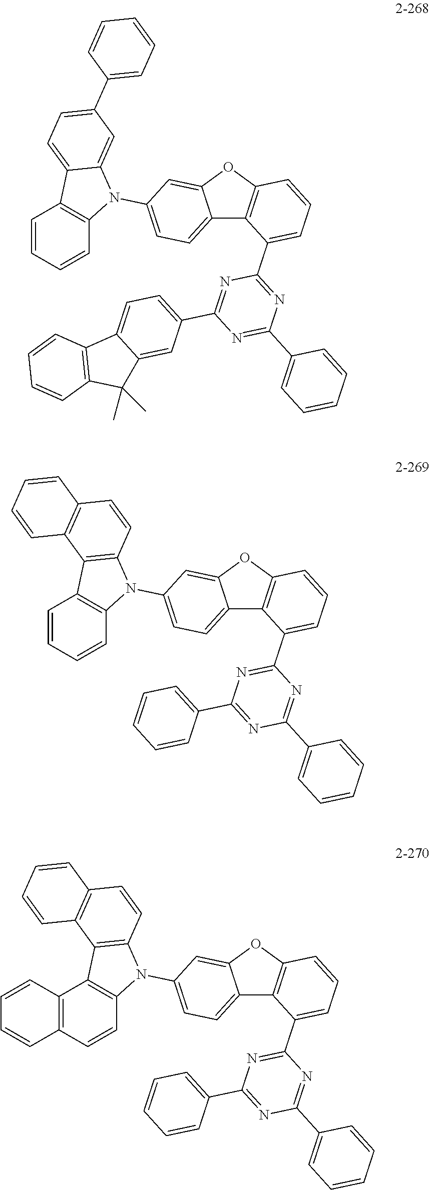 [SMILES: CC1(C)c2ccccc2-c2ccc(-c3nc(-c4ccccc4)nc(-c4cccc5oc6cc(-n7c8ccccc8c8ccc(-c9ccccc9)cc87)ccc6c45)n3)cc21 ; c1ccc(-c2nc(-c3ccccc3)nc(-c3cccc4oc5cc(-n6c7ccccc7c7c8ccccc8ccc76)ccc5c34)n2)cc1 ; c1ccc(-c2nc(-c3ccccc3)nc(-c3cccc4oc5cc(-n6c7ccc8ccccc8c7c7c8ccccc8ccc76)ccc5c34)n2)cc1]

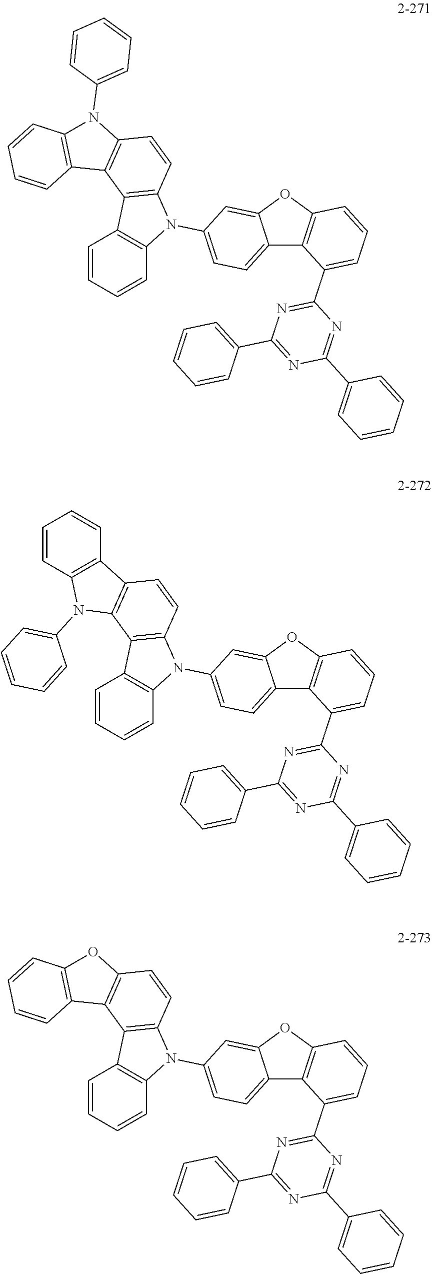 [SMILES: c1ccc(-c2nc(-c3ccccc3)nc(-c3cccc4oc5cc(-n6c7ccccc7c7c8c9ccccc9n(-c9ccccc9)c8ccc76)ccc5c34)n2)cc1 ; c1ccc(-c2nc(-c3ccccc3)nc(-c3cccc4oc5cc(-n6c7ccccc7c7c6ccc6c8ccccc8n(-c8ccccc8)c67)ccc5c34)n2)cc1 ; c1ccc(-c2nc(-c3ccccc3)nc(-c3cccc4oc5cc(-n6c7ccccc7c7c8c(ccc76)oc6ccccc68)ccc5c34)n2)cc1]

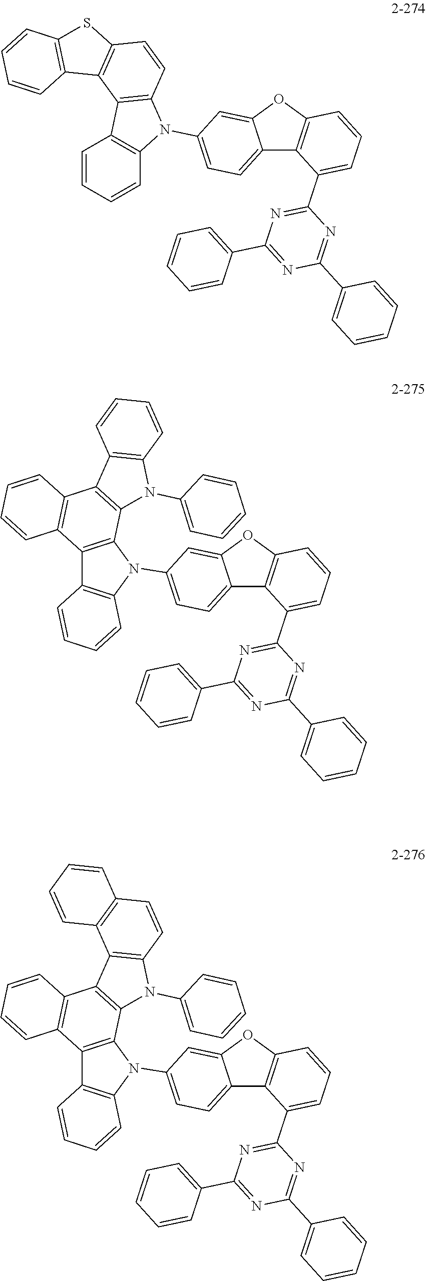 [SMILES: c1ccc(-c2nc(-c3ccccc3)nc(-c3cccc4oc5cc(-n6c7ccccc7c7c8c(ccc76)sc6ccccc68)ccc5c34)n2)cc1 ; c1ccc(-c2nc(-c3ccccc3)nc(-c3cccc4oc5cc(-n6c7ccccc7c7c8ccccc8c8c9ccccc9n(-c9ccccc9)c8c76)ccc5c34)n2)cc1 ; c1ccc(-c2nc(-c3ccccc3)nc(-c3cccc4oc5cc(-n6c7ccccc7c7c8ccccc8c8c9c%10ccccc%10ccc9n(-c9ccccc9)c8c76)ccc5c34)n2)cc1]

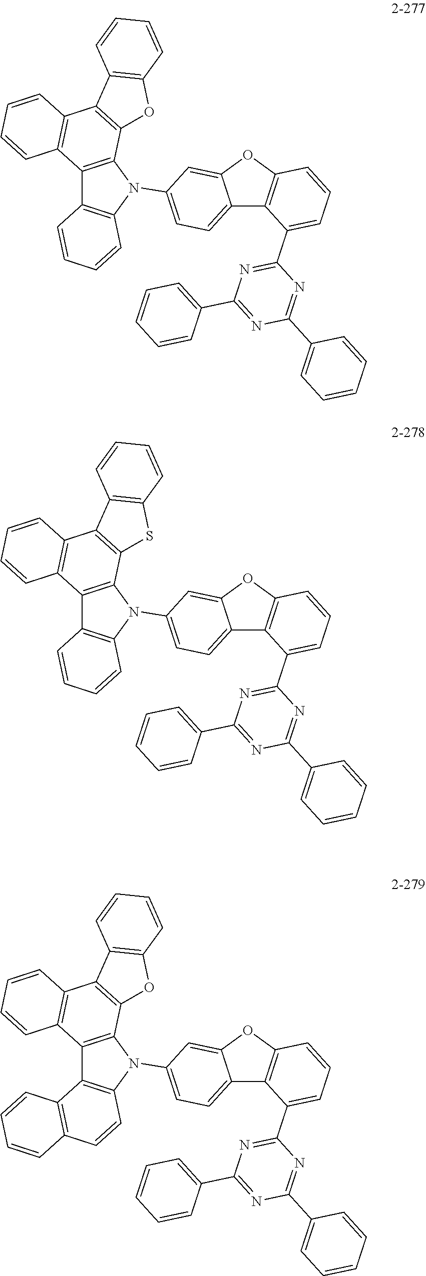 [SMILES: c1ccc(-c2nc(-c3ccccc3)nc(-c3cccc4oc5cc(-n6c7ccccc7c7c8ccccc8c8c9ccccc9oc8c76)ccc5c34)n2)cc1 ; c1ccc(-c2nc(-c3ccccc3)nc(-c3cccc4oc5cc(-n6c7ccccc7c7c8ccccc8c8c9ccccc9sc8c76)ccc5c34)n2)cc1 ; c1ccc(-c2nc(-c3ccccc3)nc(-c3cccc4oc5cc(-n6c7ccc8ccccc8c7c7c8ccccc8c8c9ccccc9oc8c76)ccc5c34)n2)cc1]

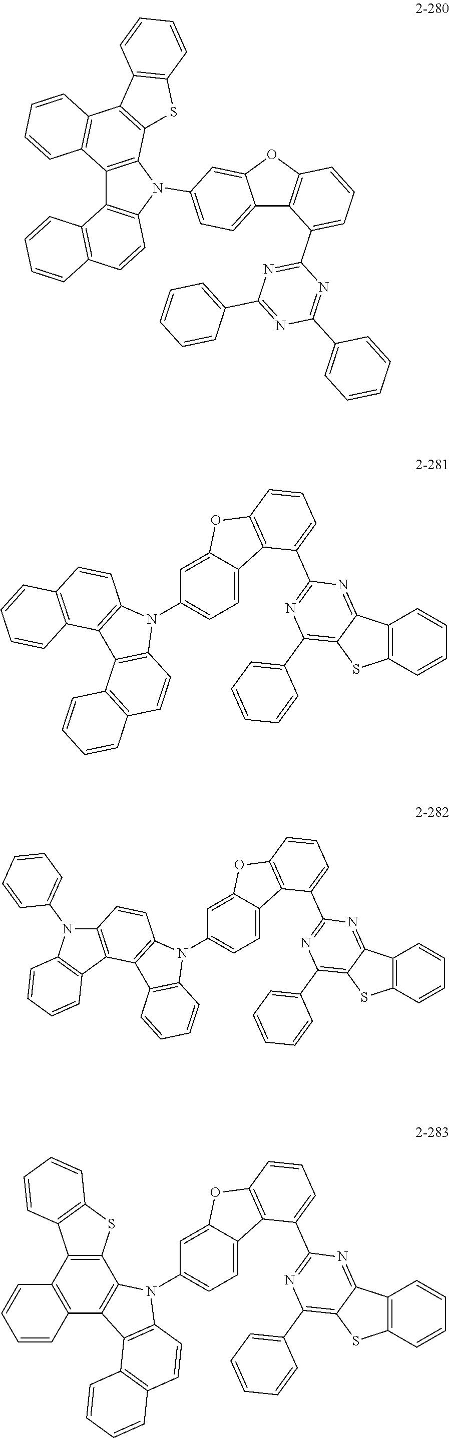 [SMILES: c1ccc(-c2nc(-c3ccccc3)nc(-c3cccc4oc5cc(-n6c7ccc8ccccc8c7c7c8ccccc8c8c9ccccc9sc8c76)ccc5c34)n2)cc1 ; c1ccc(-c2nc(-c3cccc4oc5cc(-n6c7ccc8ccccc8c7c7c8ccccc8ccc76)ccc5c34)nc3c2sc2ccccc23)cc1 ; c1ccc(-c2nc(-c3cccc4oc5cc(-n6c7ccccc7c7c8c9ccccc9n(-c9ccccc9)c8ccc76)ccc5c34)nc3c2sc2ccccc23)cc1 ; c1ccc(-c2nc(-c3cccc4oc5cc(-n6c7ccc8ccccc8c7c7c8ccccc8c8c9ccccc9sc8c76)ccc5c34)nc3c2sc2ccccc23)cc1]

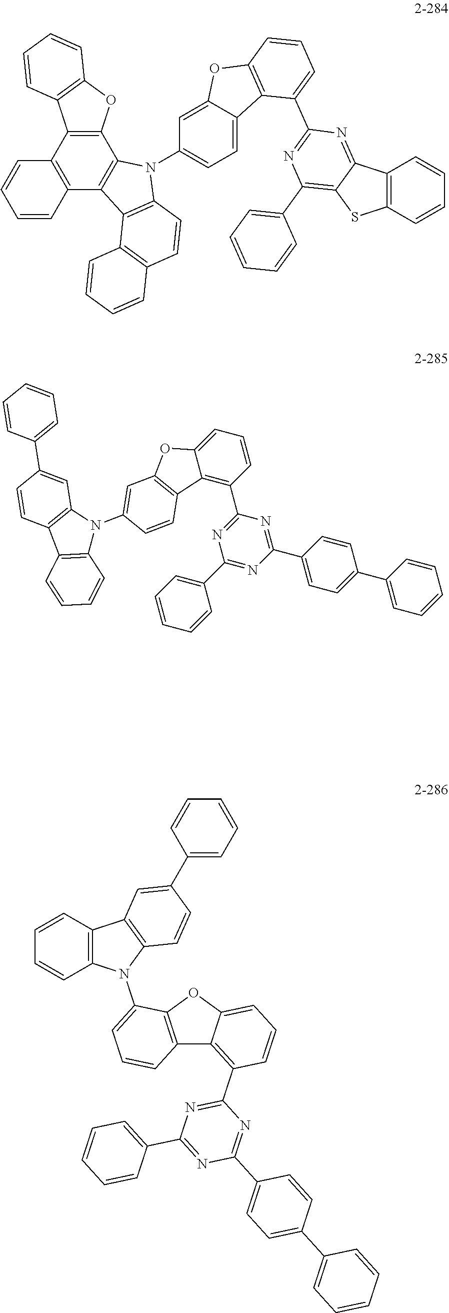 [SMILES: c1ccc(-c2nc(-c3cccc4oc5cc(-n6c7ccc8ccccc8c7c7c8ccccc8c8c9ccccc9oc8c76)ccc5c34)nc3c2sc2ccccc23)cc1 ; c1ccc(-c2ccc(-c3nc(-c4ccccc4)nc(-c4cccc5oc6cc(-n7c8ccccc8c8ccc(-c9ccccc9)cc87)ccc6c45)n3)cc2)cc1 ; c1ccc(-c2ccc(-c3nc(-c4ccccc4)nc(-c4cccc5oc6c(-n7c8ccccc8c8cc(-c9ccccc9)ccc87)cccc6c45)n3)cc2)cc1]

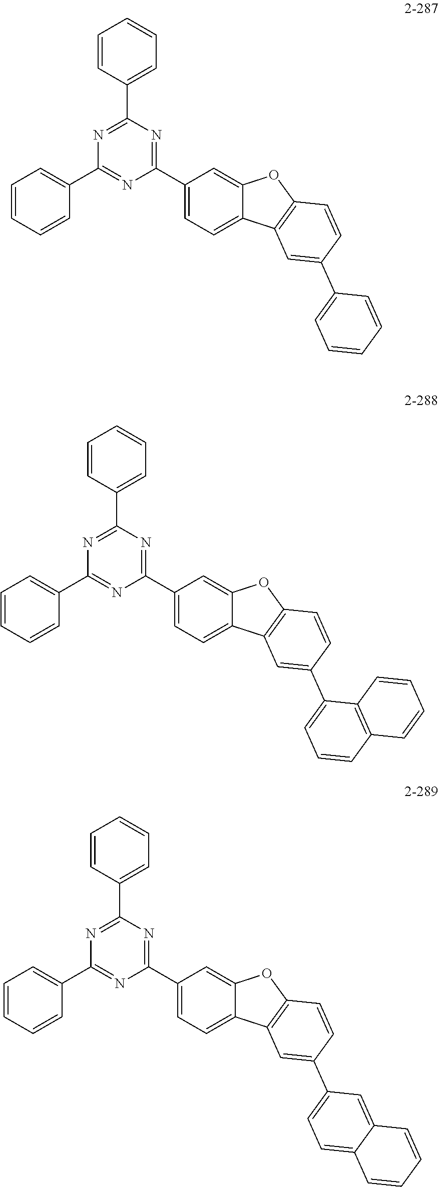 [SMILES: c1ccc(-c2ccc3oc4cc(-c5nc(-c6ccccc6)nc(-c6ccccc6)n5)ccc4c3c2)cc1 ; c1ccc(-c2nc(-c3ccccc3)nc(-c3ccc4c(c3)oc3ccc(-c5cccc6ccccc56)cc34)n2)cc1 ; c1ccc(-c2nc(-c3ccccc3)nc(-c3ccc4c(c3)oc3ccc(-c5ccc6ccccc6c5)cc34)n2)cc1]

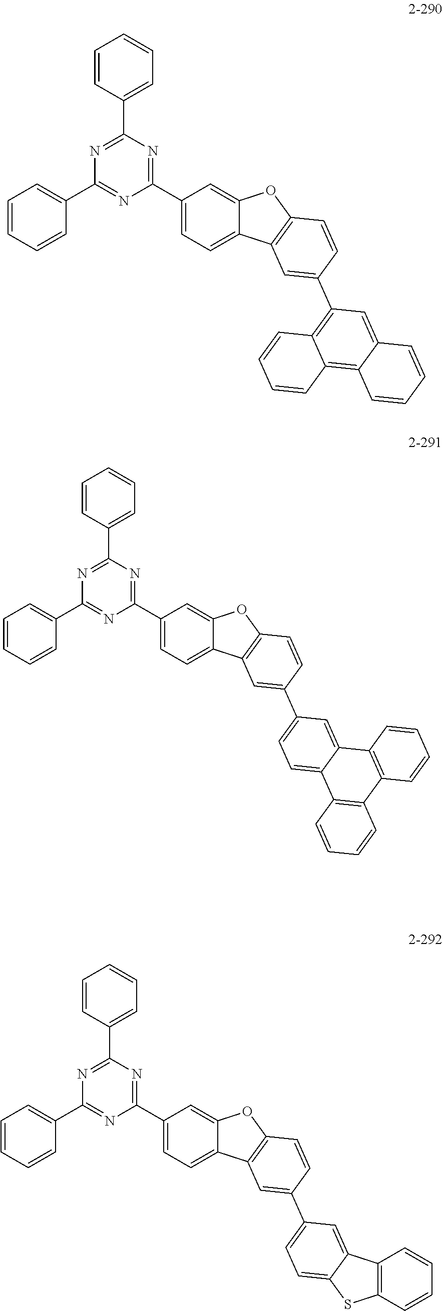 [SMILES: c1ccc(-c2nc(-c3ccccc3)nc(-c3ccc4c(c3)oc3ccc(-c5cc6ccccc6c6ccccc56)cc34)n2)cc1 ; c1ccc(-c2nc(-c3ccccc3)nc(-c3ccc4c(c3)oc3ccc(-c5ccc6c7ccccc7c7ccccc7c6c5)cc34)n2)cc1 ; c1ccc(-c2nc(-c3ccccc3)nc(-c3ccc4c(c3)oc3ccc(-c5ccc6sc7ccccc7c6c5)cc34)n2)cc1]

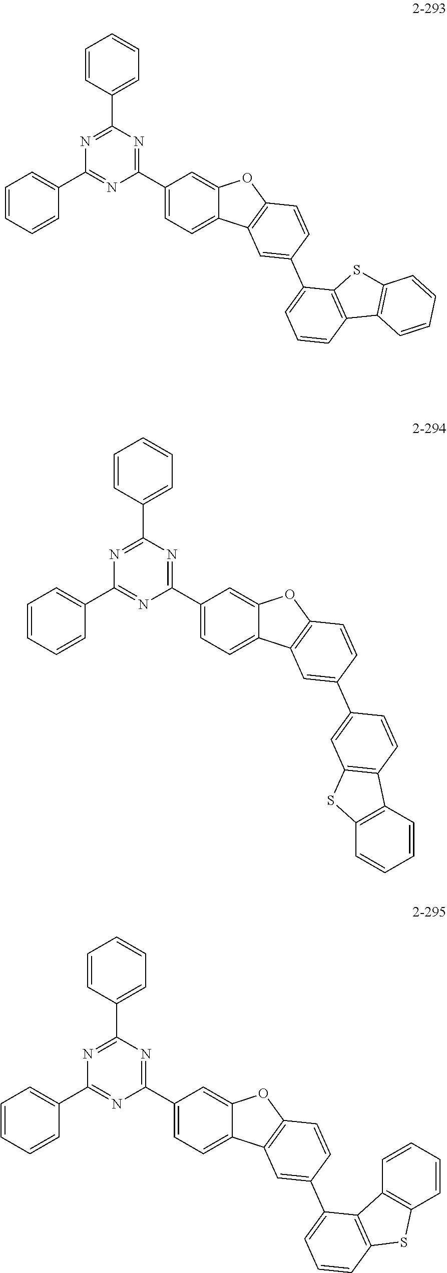 [SMILES: c1ccc(-c2nc(-c3ccccc3)nc(-c3ccc4c(c3)oc3ccc(-c5cccc6c5sc5ccccc56)cc34)n2)cc1 ; c1ccc(-c2nc(-c3ccccc3)nc(-c3ccc4c(c3)oc3ccc(-c5ccc6c(c5)sc5ccccc56)cc34)n2)cc1 ; c1ccc(-c2nc(-c3ccccc3)nc(-c3ccc4c(c3)oc3ccc(-c5cccc6sc7ccccc7c56)cc34)n2)cc1]

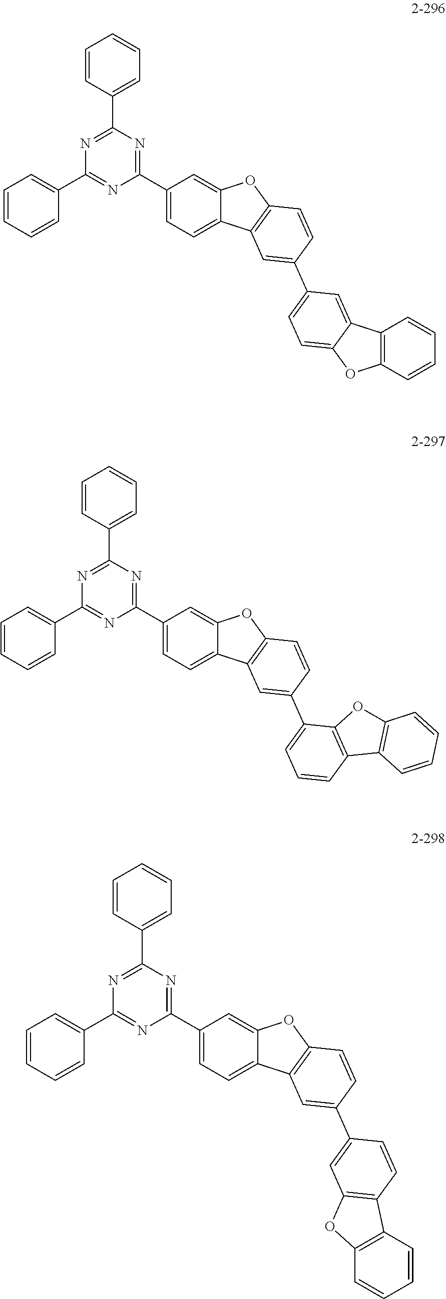 [SMILES: c1ccc(-c2nc(-c3ccccc3)nc(-c3ccc4c(c3)oc3ccc(-c5ccc6oc7ccccc7c6c5)cc34)n2)cc1 ; c1ccc(-c2nc(-c3ccccc3)nc(-c3ccc4c(c3)oc3ccc(-c5cccc6c5oc5ccccc56)cc34)n2)cc1 ; c1ccc(-c2nc(-c3ccccc3)nc(-c3ccc4c(c3)oc3ccc(-c5ccc6c(c5)oc5ccccc56)cc34)n2)cc1]

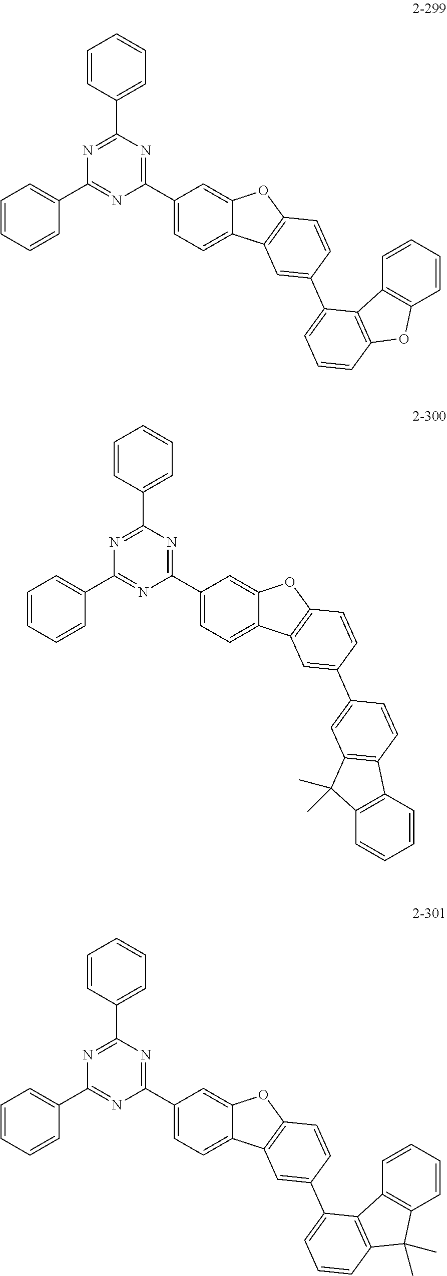 [SMILES: c1ccc(-c2nc(-c3ccccc3)nc(-c3ccc4c(c3)oc3ccc(-c5cccc6oc7ccccc7c56)cc34)n2)cc1 ; CC1(C)c2ccccc2-c2ccc(-c3ccc4oc5cc(-c6nc(-c7ccccc7)nc(-c7ccccc7)n6)ccc5c4c3)cc21 ; CC1(C)c2ccccc2-c2c(-c3ccc4oc5cc(-c6nc(-c7ccccc7)nc(-c7ccccc7)n6)ccc5c4c3)cccc21]

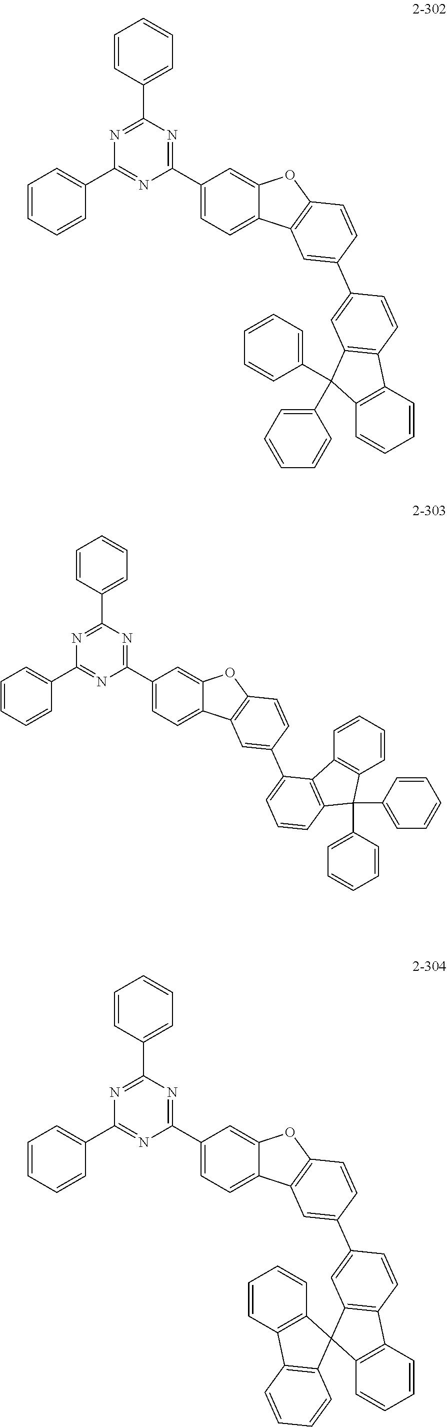 [SMILES: c1ccc(-c2nc(-c3ccccc3)nc(-c3ccc4c(c3)oc3ccc(-c5ccc6c(c5)C(c5ccccc5)(c5ccccc5)c5ccccc5-6)cc34)n2)cc1 ; c1ccc(-c2nc(-c3ccccc3)nc(-c3ccc4c(c3)oc3ccc(-c5cccc6c5-c5ccccc5C6(c5ccccc5)c5ccccc5)cc34)n2)cc1 ; c1ccc(-c2nc(-c3ccccc3)nc(-c3ccc4c(c3)oc3ccc(-c5ccc6c(c5)C5(c7ccccc7-c7ccccc75)c5ccccc5-6)cc34)n2)cc1]

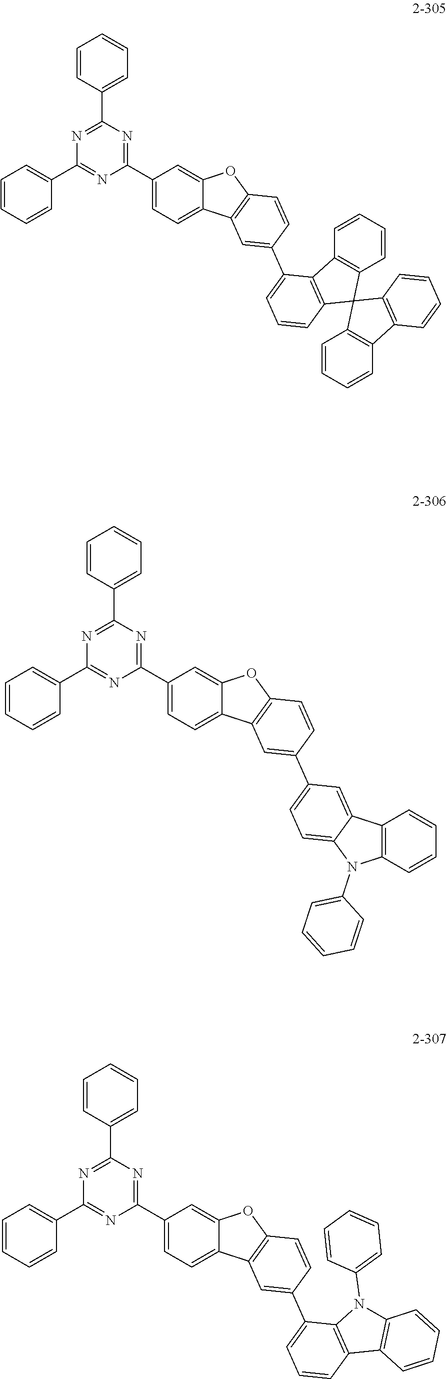 [SMILES: c1ccc(-c2nc(-c3ccccc3)nc(-c3ccc4c(c3)oc3ccc(-c5cccc6c5-c5ccccc5C65c6ccccc6-c6ccccc65)cc34)n2)cc1 ; c1ccc(-c2nc(-c3ccccc3)nc(-c3ccc4c(c3)oc3ccc(-c5ccc6c(c5)c5ccccc5n6-c5ccccc5)cc34)n2)cc1 ; c1ccc(-c2nc(-c3ccccc3)nc(-c3ccc4c(c3)oc3ccc(-c5cccc6c7ccccc7n(-c7ccccc7)c56)cc34)n2)cc1]

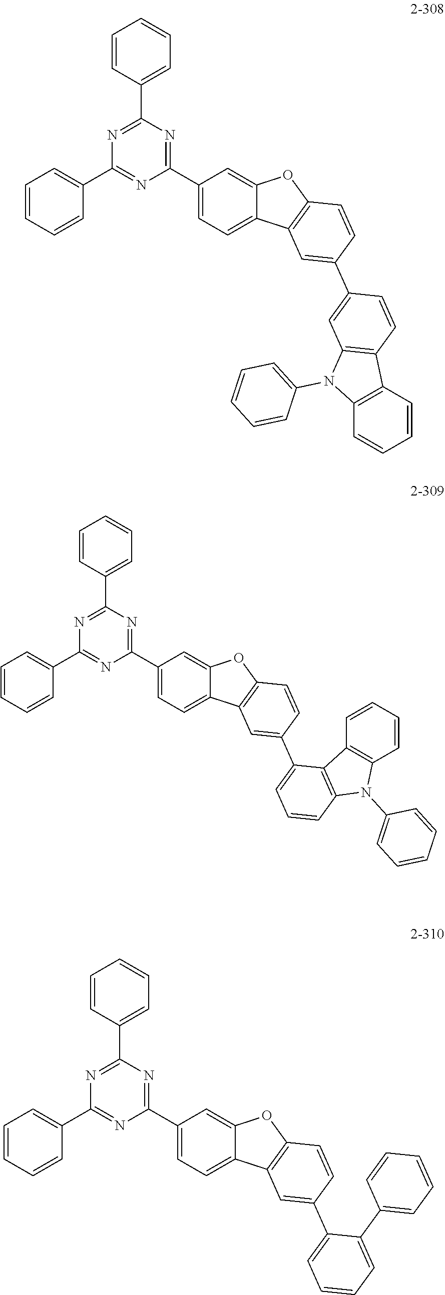 [SMILES: c1ccc(-c2nc(-c3ccccc3)nc(-c3ccc4c(c3)oc3ccc(-c5ccc6c7ccccc7n(-c7ccccc7)c6c5)cc34)n2)cc1 ; c1ccc(-c2nc(-c3ccccc3)nc(-c3ccc4c(c3)oc3ccc(-c5cccc6c5c5ccccc5n6-c5ccccc5)cc34)n2)cc1 ; c1ccc(-c2nc(-c3ccccc3)nc(-c3ccc4c(c3)oc3ccc(-c5ccccc5-c5ccccc5)cc34)n2)cc1]

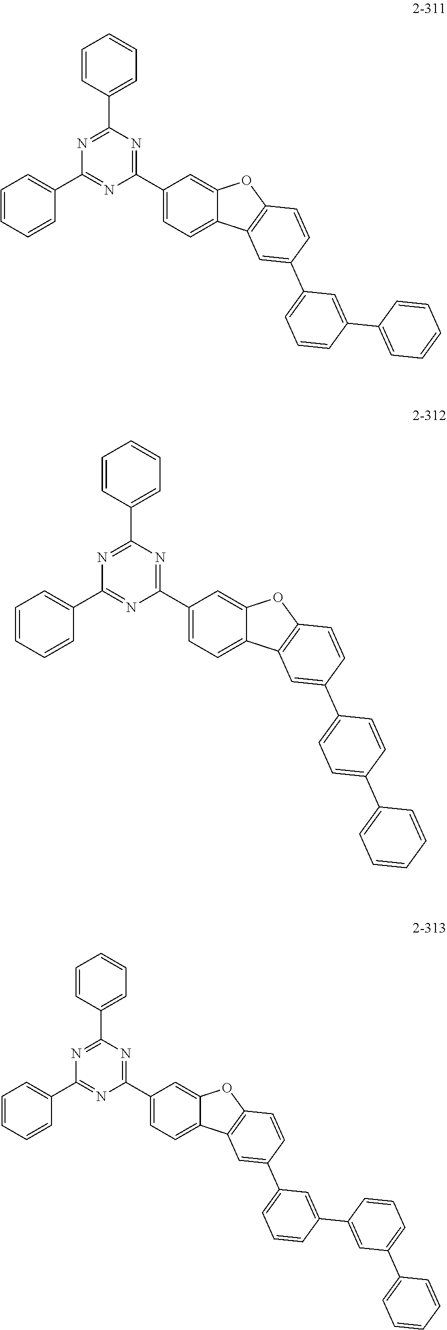 [SMILES: c1ccc(-c2cccc(-c3ccc4oc5cc(-c6nc(-c7ccccc7)nc(-c7ccccc7)n6)ccc5c4c3)c2)cc1 ; c1ccc(-c2ccc(-c3ccc4oc5cc(-c6nc(-c7ccccc7)nc(-c7ccccc7)n6)ccc5c4c3)cc2)cc1 ; c1ccc(-c2cccc(-c3cccc(-c4ccc5oc6cc(-c7nc(-c8ccccc8)nc(-c8ccccc8)n7)ccc6c5c4)c3)c2)cc1]

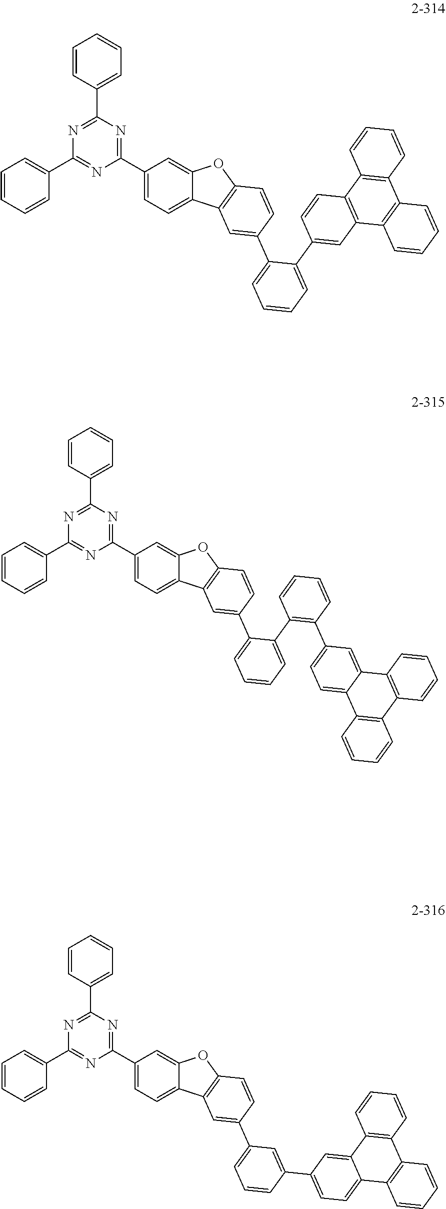 [SMILES: c1ccc(-c2nc(-c3ccccc3)nc(-c3ccc4c(c3)oc3ccc(-c5ccccc5-c5ccc6c7ccccc7c7ccccc7c6c5)cc34)n2)cc1 ; c1ccc(-c2nc(-c3ccccc3)nc(-c3ccc4c(c3)oc3ccc(-c5ccccc5-c5ccccc5-c5ccc6c7ccccc7c7ccccc7c6c5)cc34)n2)cc1 ; c1ccc(-c2nc(-c3ccccc3)nc(-c3ccc4c(c3)oc3ccc(-c5cccc(-c6ccc7c8ccccc8c8ccccc8c7c6)c5)cc34)n2)cc1]

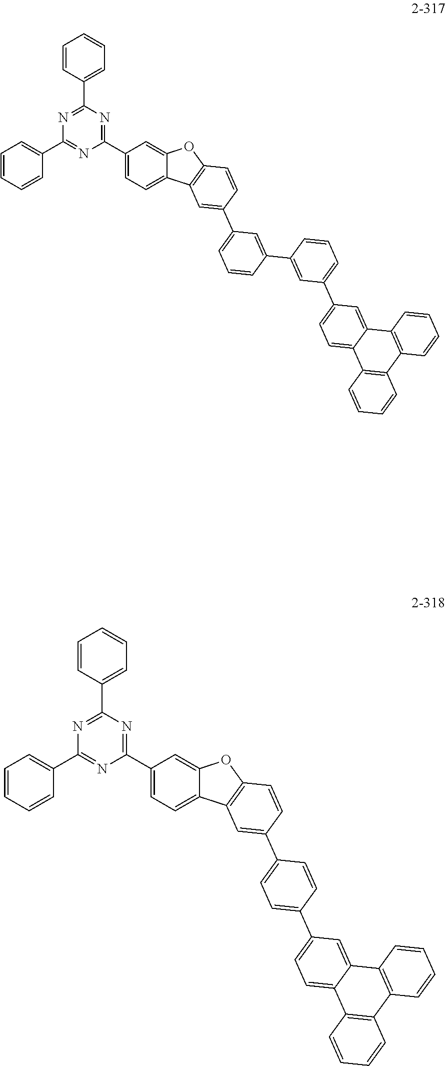 [SMILES: c1ccc(-c2nc(-c3ccccc3)nc(-c3ccc4c(c3)oc3ccc(-c5cccc(-c6cccc(-c7ccc8c9ccccc9c9ccccc9c8c7)c6)c5)cc34)n2)cc1 ; c1ccc(-c2nc(-c3ccccc3)nc(-c3ccc4c(c3)oc3ccc(-c5ccc(-c6ccc7c8ccccc8c8ccccc8c7c6)cc5)cc34)n2)cc1]

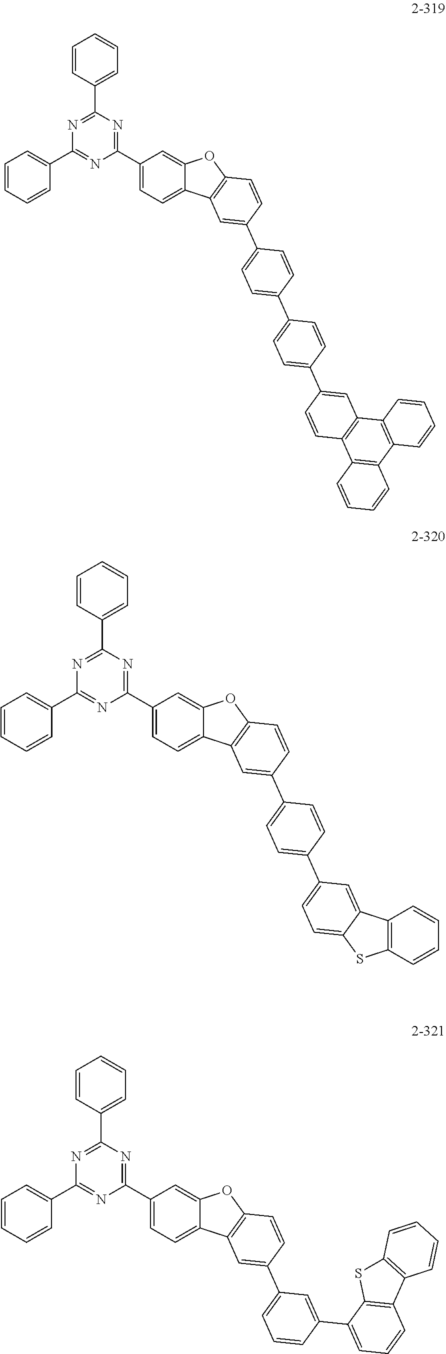 [SMILES: c1ccc(-c2nc(-c3ccccc3)nc(-c3ccc4c(c3)oc3ccc(-c5ccc(-c6ccc(-c7ccc8c9ccccc9c9ccccc9c8c7)cc6)cc5)cc34)n2)cc1 ; c1ccc(-c2nc(-c3ccccc3)nc(-c3ccc4c(c3)oc3ccc(-c5ccc(-c6ccc7sc8ccccc8c7c6)cc5)cc34)n2)cc1 ; c1ccc(-c2nc(-c3ccccc3)nc(-c3ccc4c(c3)oc3ccc(-c5cccc(-c6cccc7c6sc6ccccc67)c5)cc34)n2)cc1]

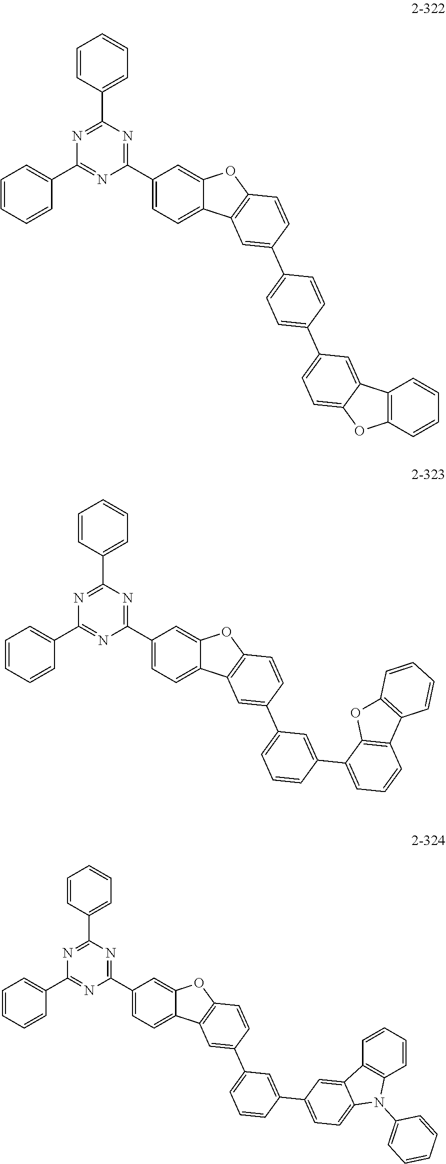 [SMILES: c1ccc(-c2nc(-c3ccccc3)nc(-c3ccc4c(c3)oc3ccc(-c5ccc(-c6ccc7oc8ccccc8c7c6)cc5)cc34)n2)cc1 ; c1ccc(-c2nc(-c3ccccc3)nc(-c3ccc4c(c3)oc3ccc(-c5cccc(-c6cccc7c6oc6ccccc67)c5)cc34)n2)cc1 ; c1ccc(-c2nc(-c3ccccc3)nc(-c3ccc4c(c3)oc3ccc(-c5cccc(-c6ccc7c(c6)c6ccccc6n7-c6ccccc6)c5)cc34)n2)cc1]

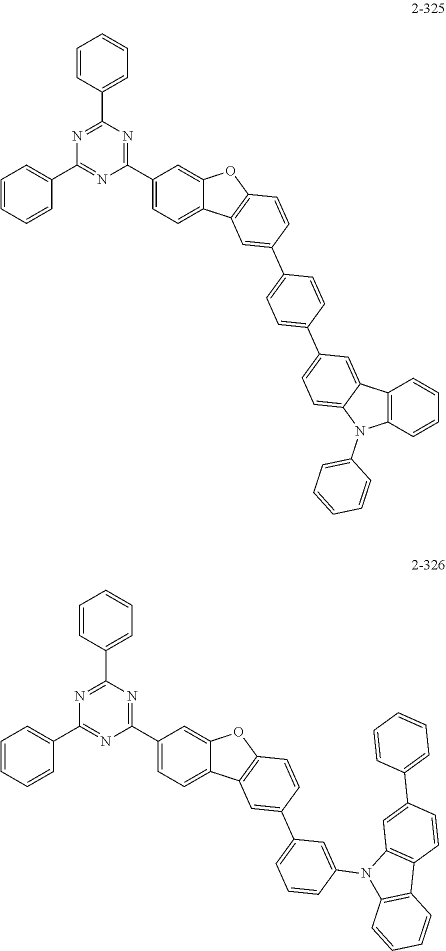 [SMILES: c1ccc(-c2nc(-c3ccccc3)nc(-c3ccc4c(c3)oc3ccc(-c5ccc(-c6ccc7c(c6)c6ccccc6n7-c6ccccc6)cc5)cc34)n2)cc1 ; c1ccc(-c2ccc3c4ccccc4n(-c4cccc(-c5ccc6oc7cc(-c8nc(-c9ccccc9)nc(-c9ccccc9)n8)ccc7c6c5)c4)c3c2)cc1]

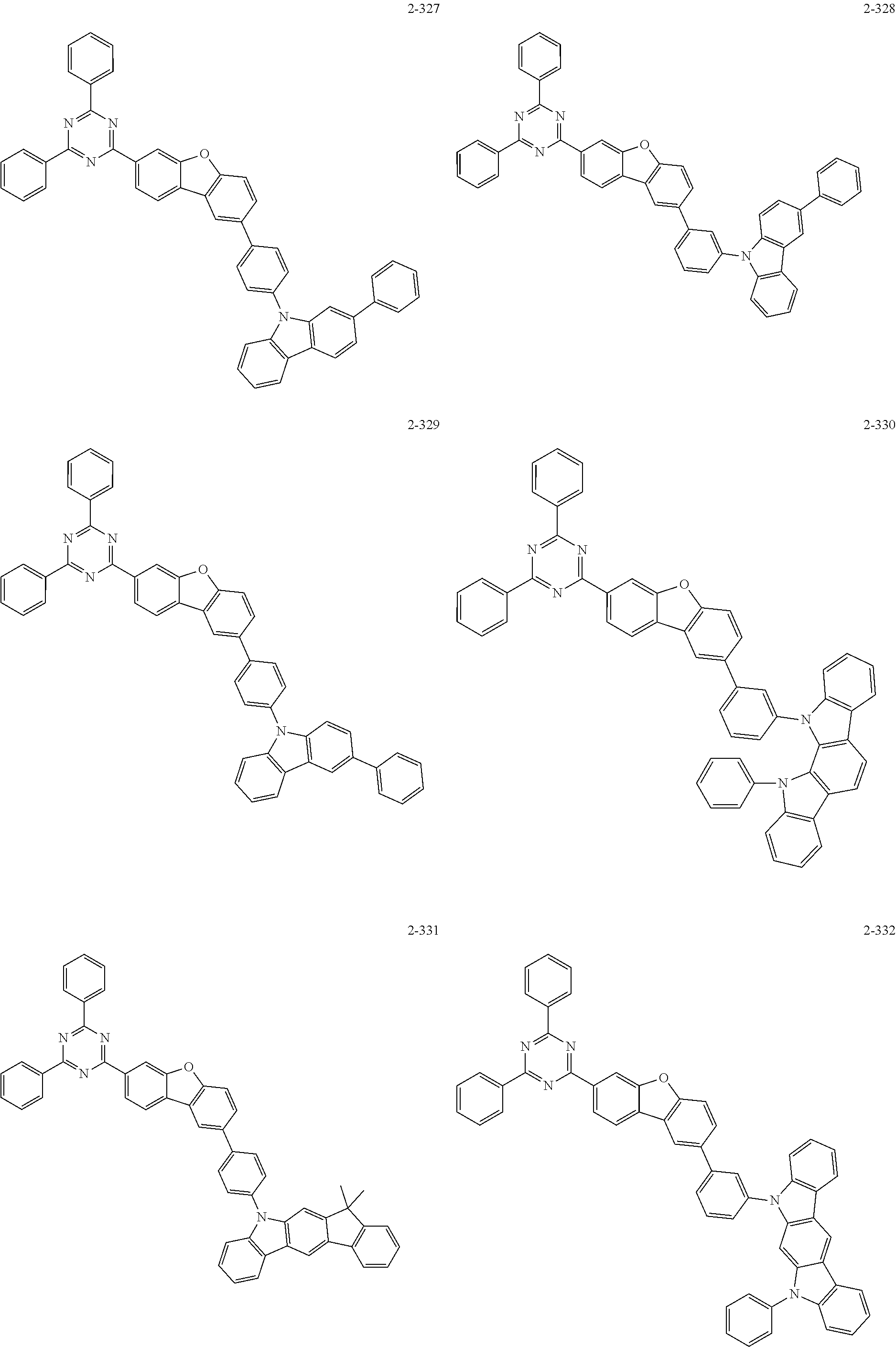

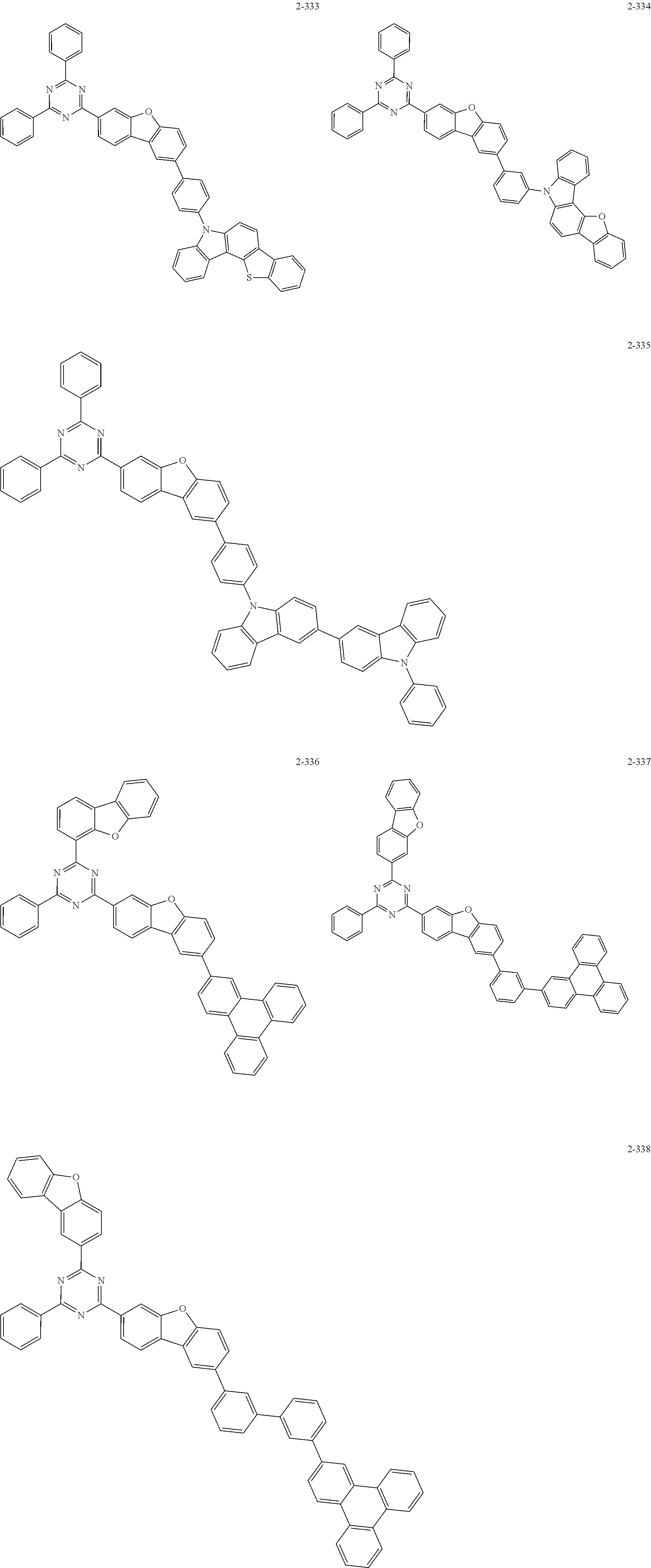

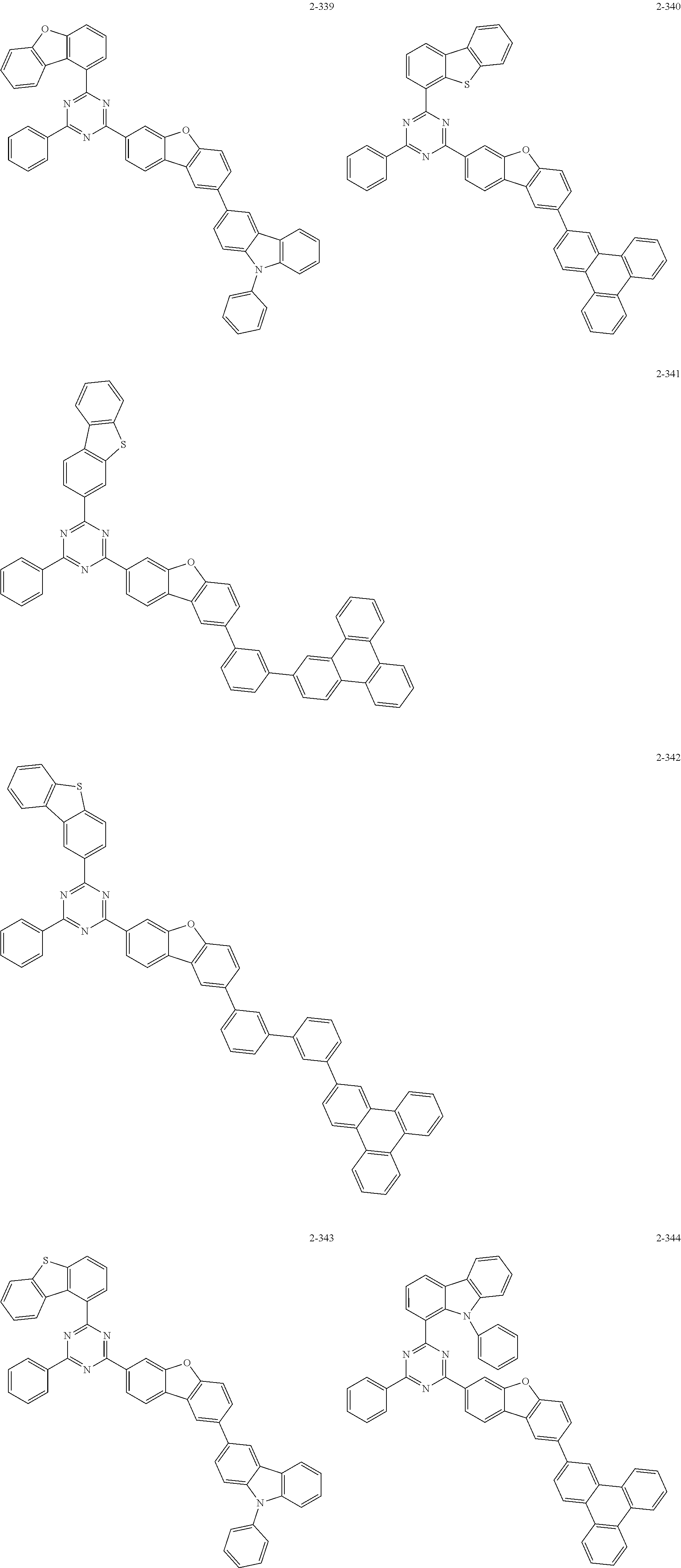

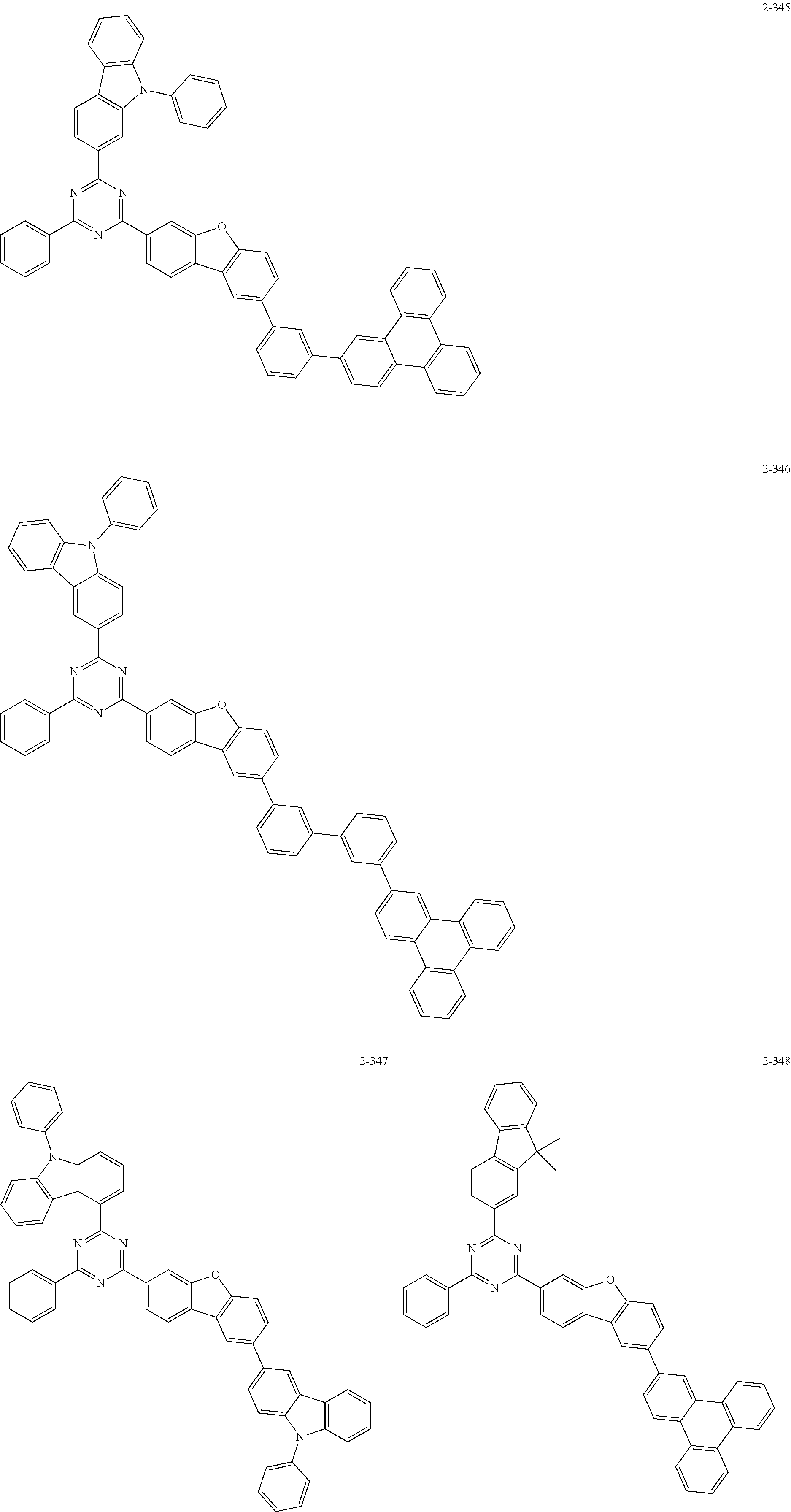 [SMILES: c1ccc(-c2nc(-c3ccc4c(c3)oc3ccc(-c5cccc(-c6ccc7c8ccccc8c8ccccc8c7c6)c5)cc34)nc(-c3ccc4c5ccccc5n(-c5ccccc5)c4c3)n2)cc1 ; c1ccc(-c2nc(-c3ccc4c(c3)oc3ccc(-c5cccc(-c6cccc(-c7ccc8c9ccccc9c9ccccc9c8c7)c6)c5)cc34)nc(-c3ccc4c(c3)c3ccccc3n4-c3ccccc3)n2)cc1 ; c1ccc(-c2nc(-c3ccc4c(c3)oc3ccc(-c5ccc6c(c5)c5ccccc5n6-c5ccccc5)cc34)nc(-c3cccc4c3c3ccccc3n4-c3ccccc3)n2)cc1 ; CC1(C)c2ccccc2-c2ccc(-c3nc(-c4ccccc4)nc(-c4ccc5c(c4)oc4ccc(-c6ccc7c8ccccc8c8ccccc8c7c6)cc45)n3)cc21]

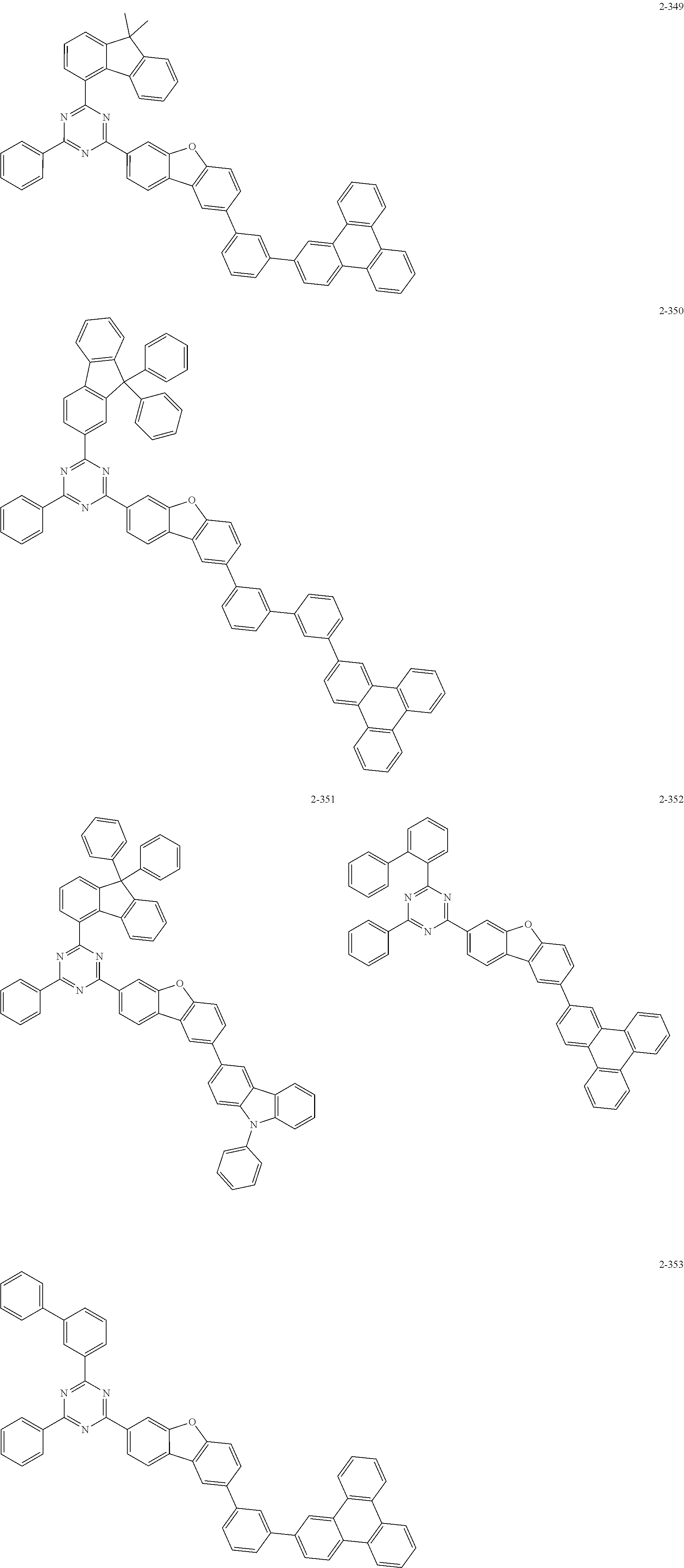

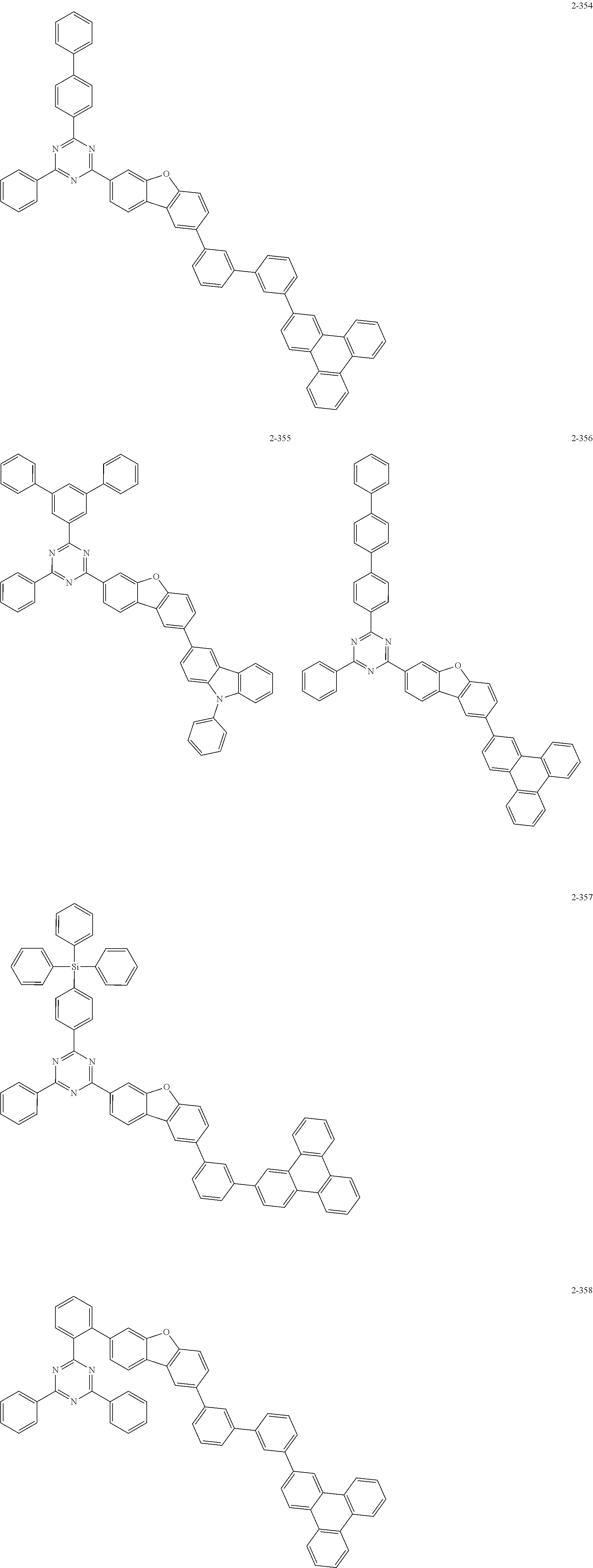

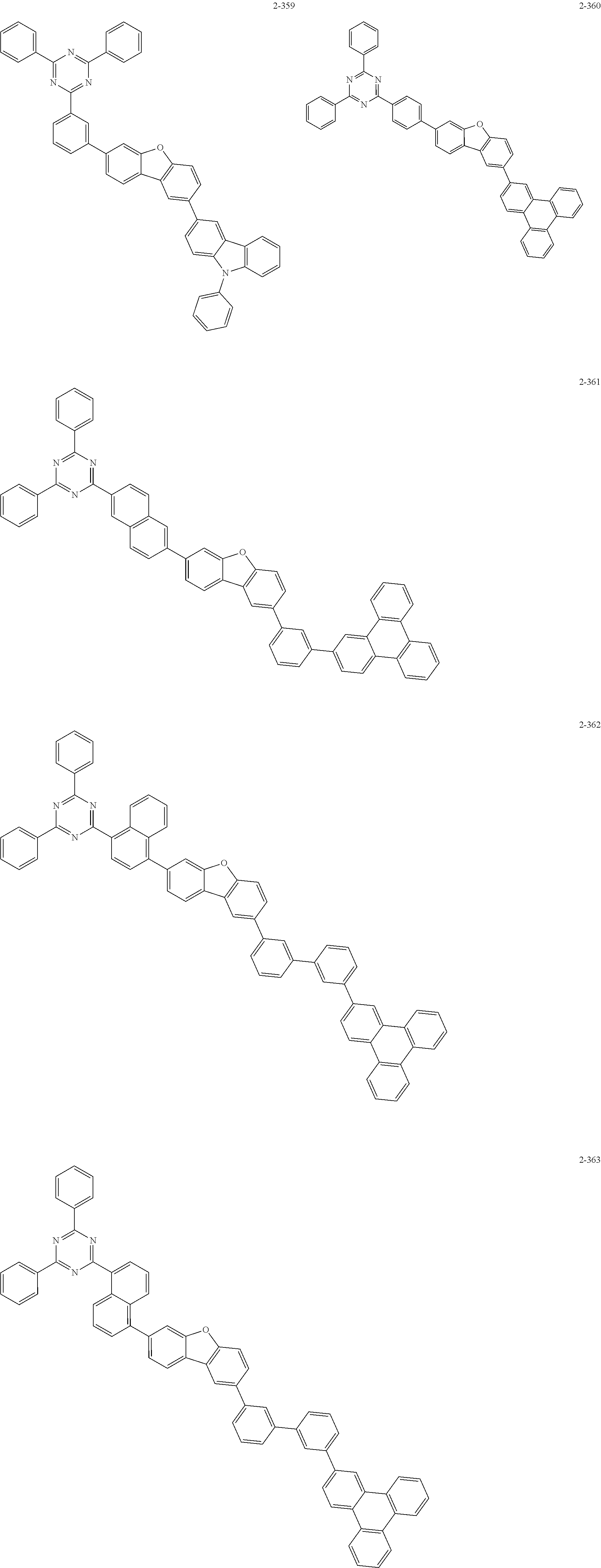

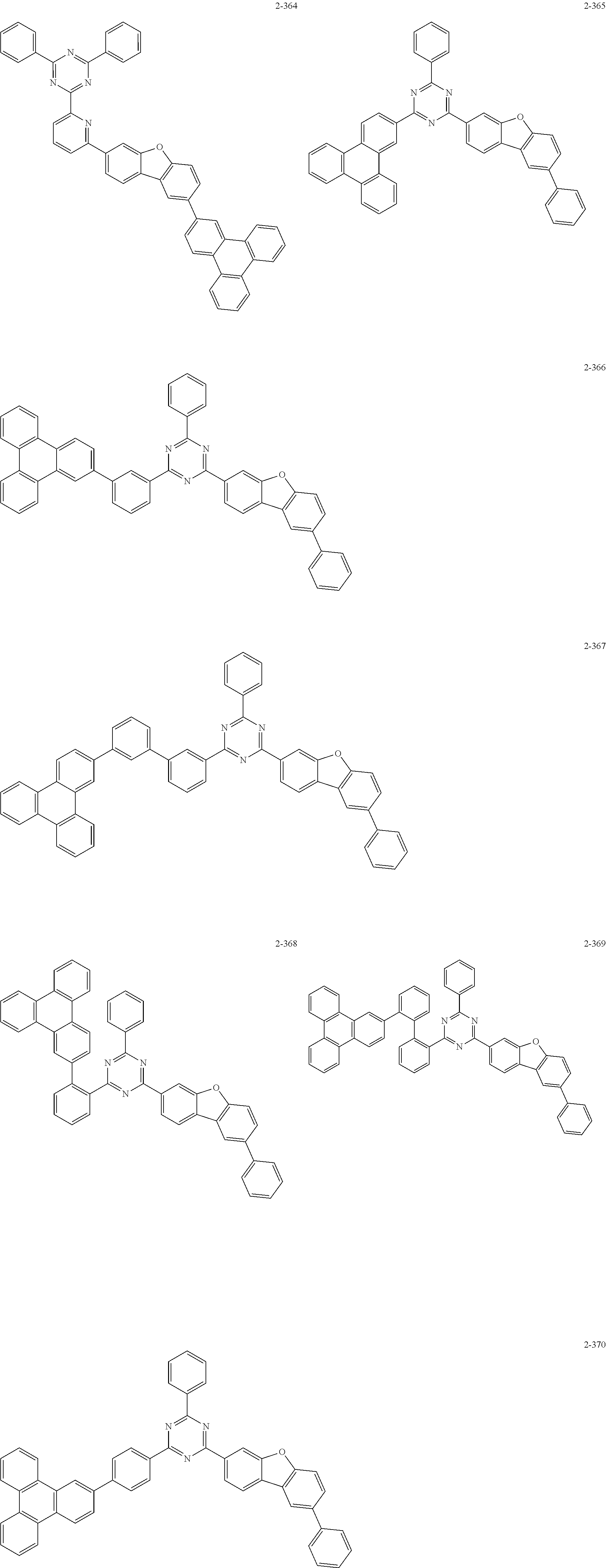

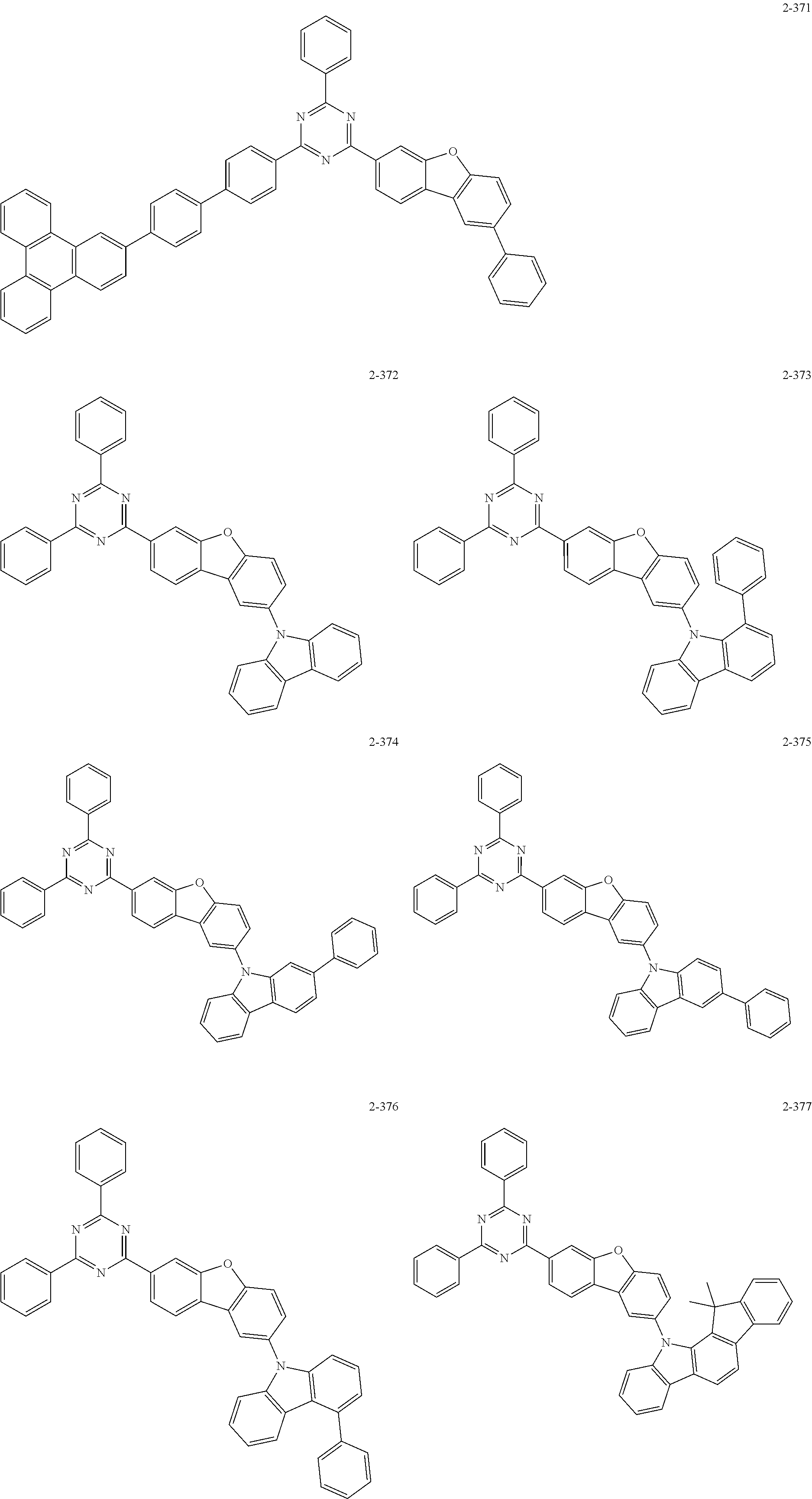

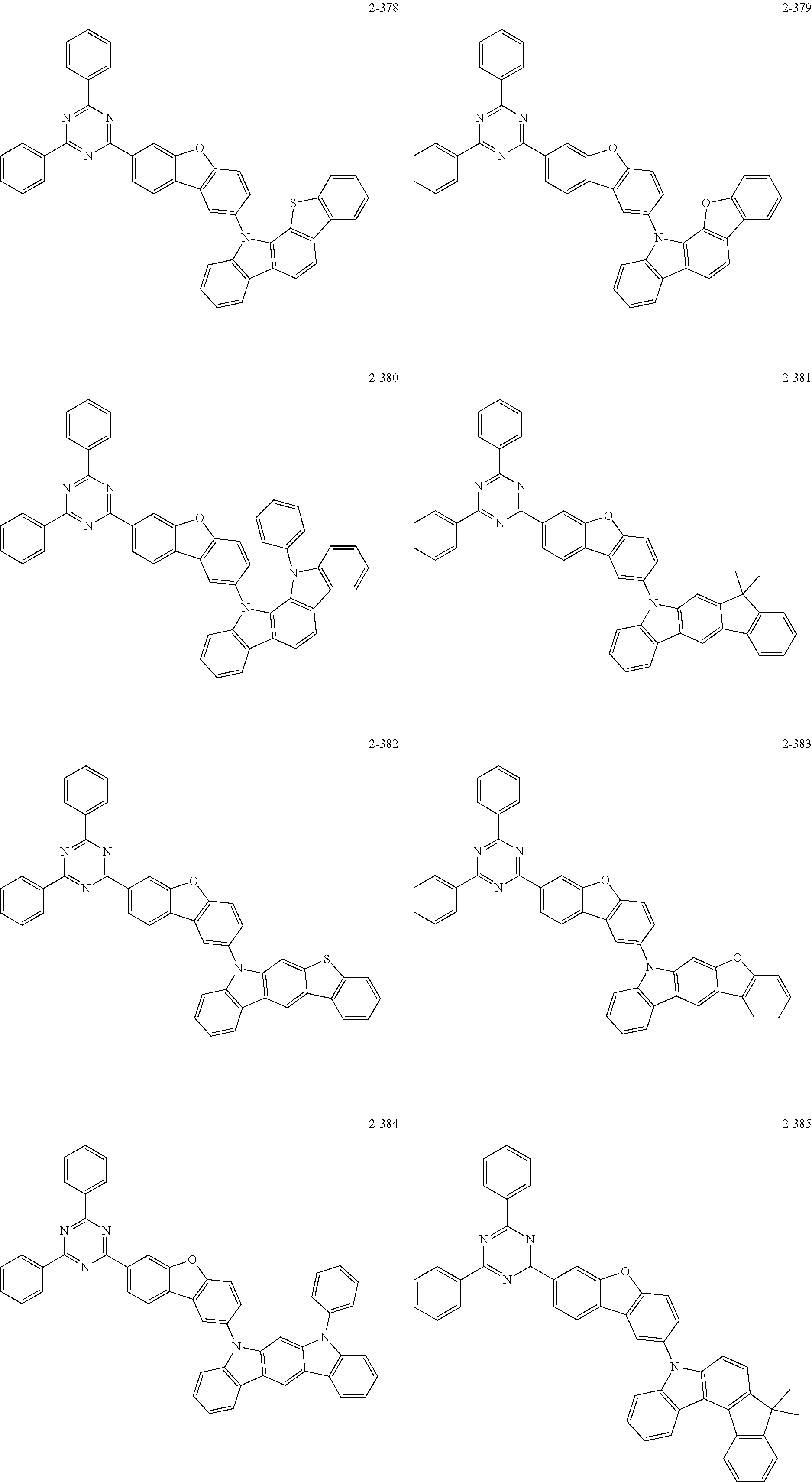

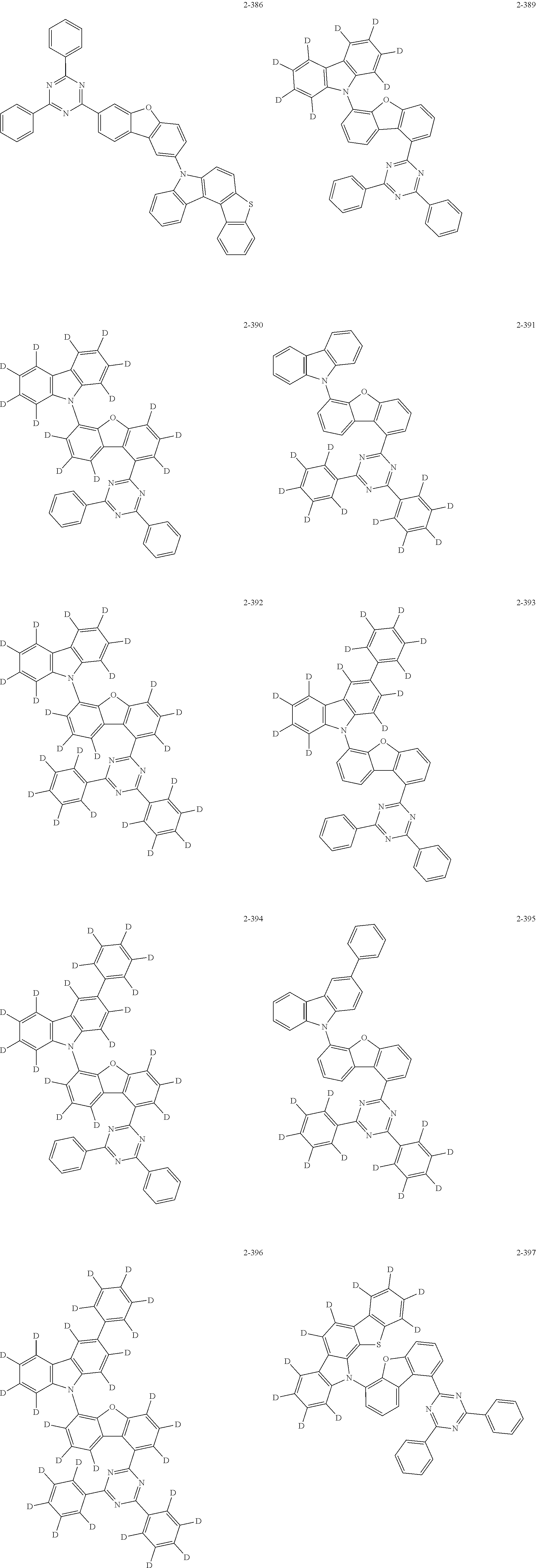

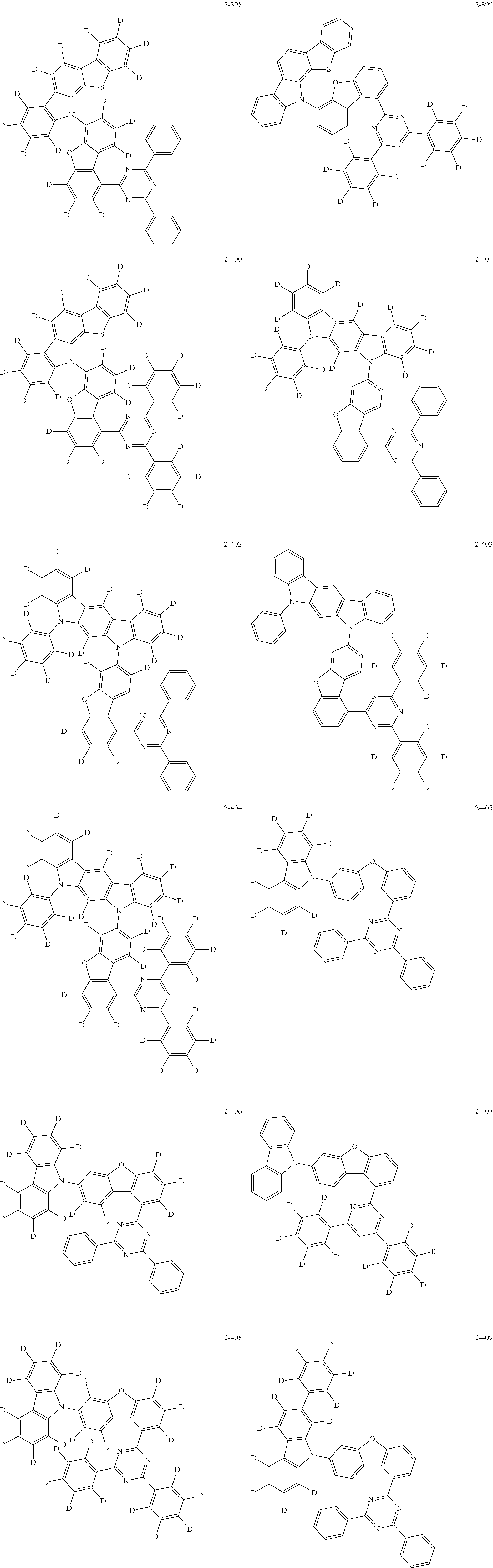

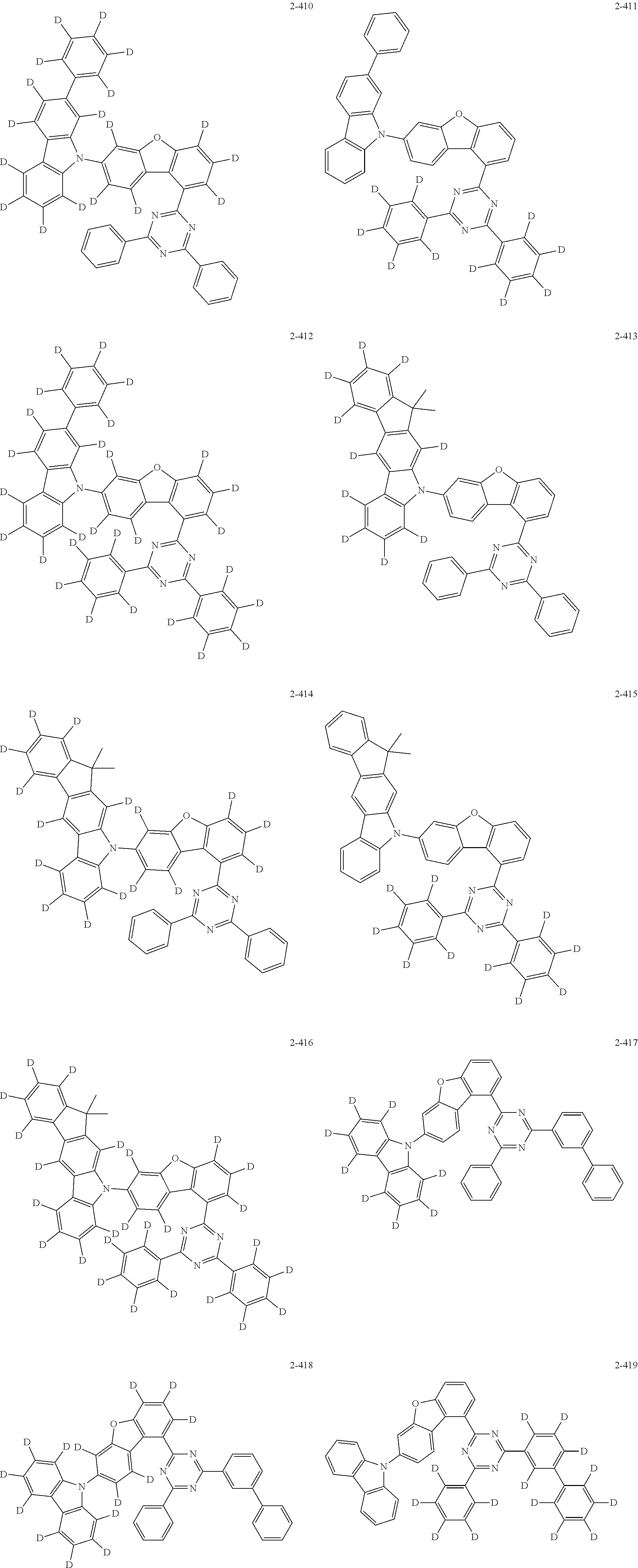

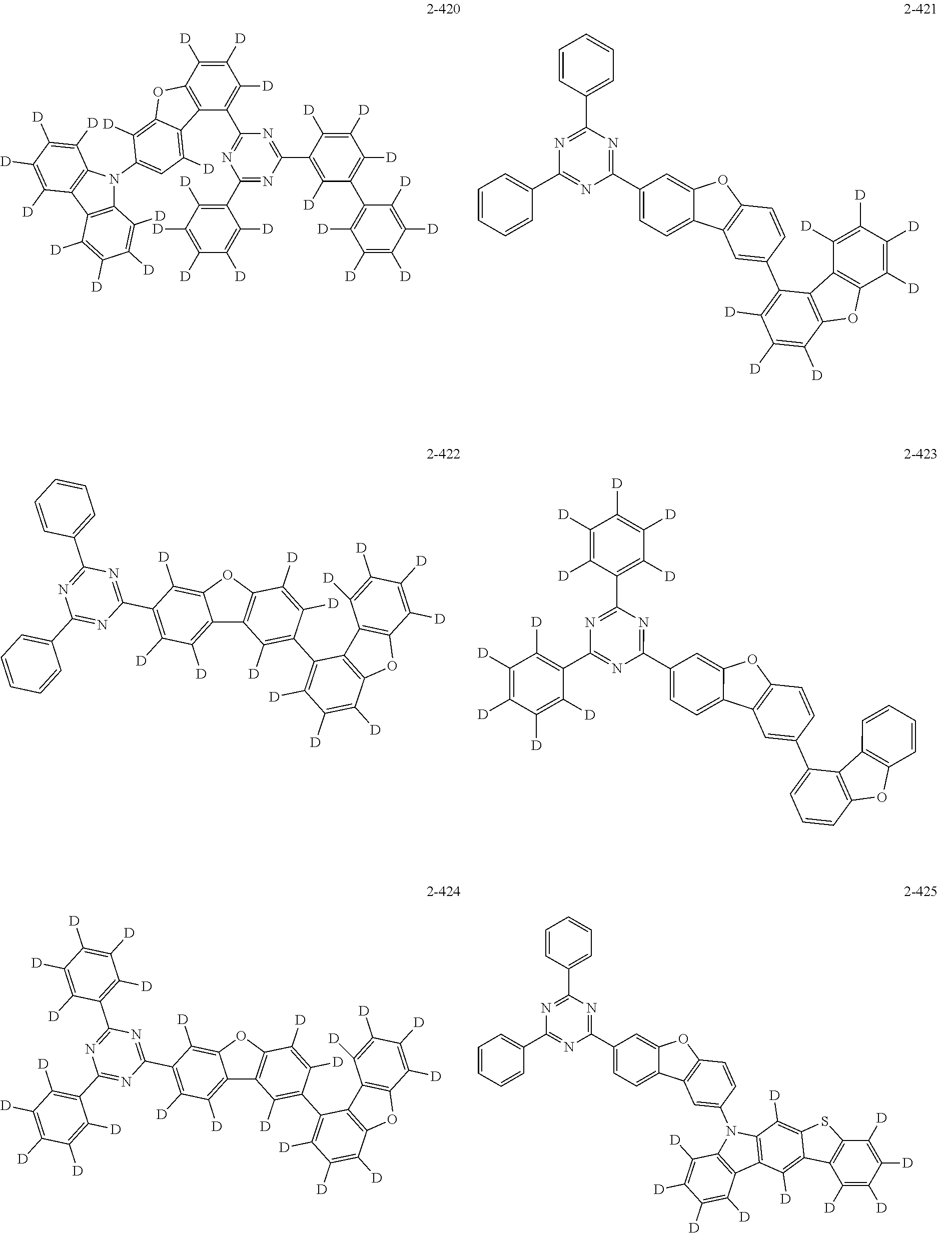

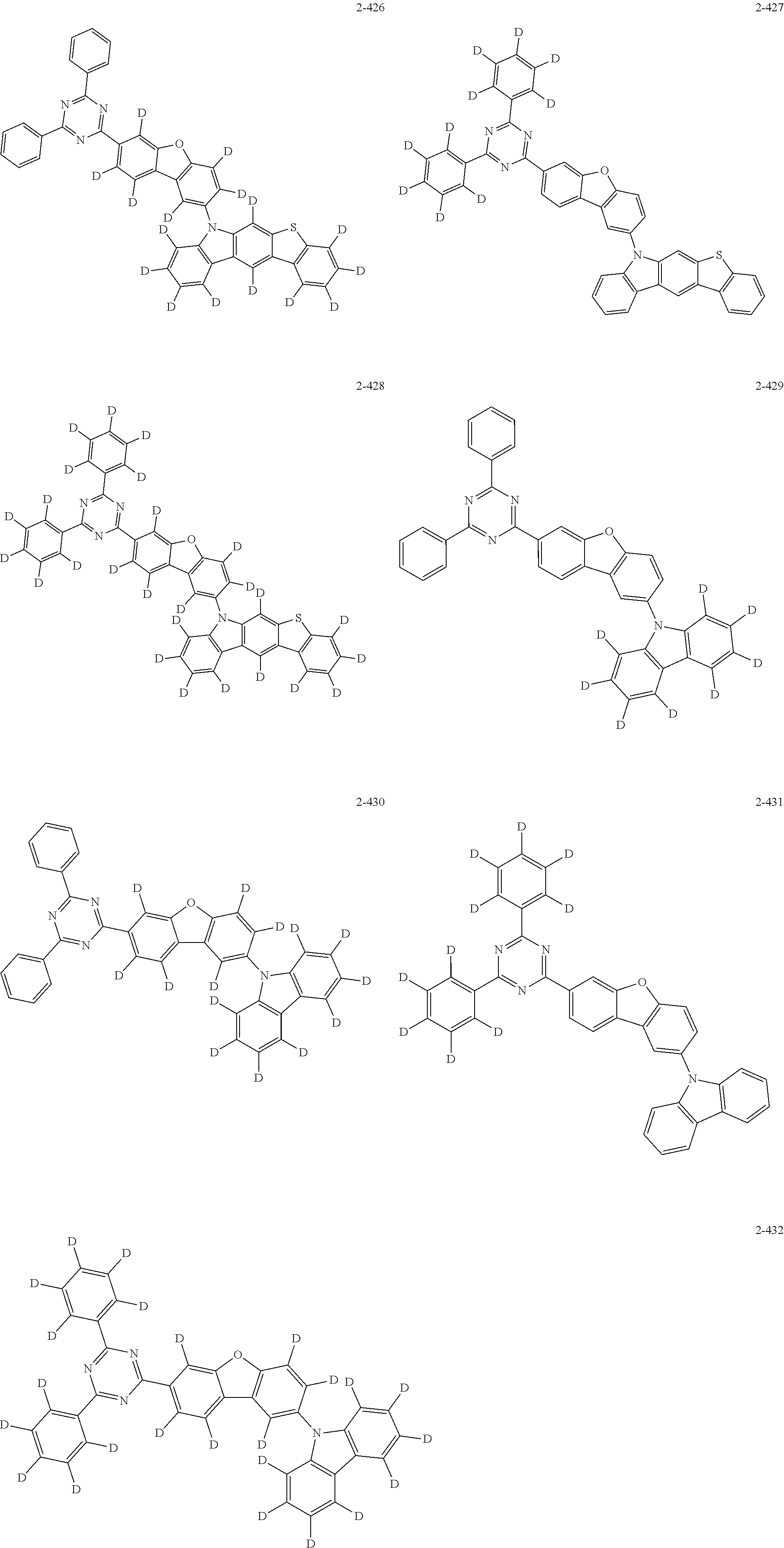

Further, various substituents may be introduced into the structure of Chemical Formula 2 to synthesize a compound having inherent characteristics of a substituent introduced. For example, it is possible to synthesize a material which satisfies conditions required for each organic material layer by introducing a substituent usually used for a hole injection layer material, a material for transporting holes, a light emitting layer material, an electron transport layer material, and a charge generation layer material, which are used for preparing an organic light emitting device, into the core structure.

In addition, it is possible to finely adjust an energy band gap by introducing various substituents into the structure of Chemical Formula 2, and meanwhile, it is possible to improve characteristics at the interface between organic materials and diversify the use of the material.

Meanwhile, the heterocyclic compound has a high glass transition temperature (Tg) and thus has excellent thermal stability. The increase in thermal stability becomes an important factor for providing a device with driving stability.

The heterocyclic compound according to an exemplary embodiment of the present application may be prepared by a multi-step chemical reaction. Some intermediate compounds are first prepared, and the compound of Chemical Formula 2 may be prepared from the intermediate compounds. More specifically, the heterocyclic compound according to an exemplary embodiment of the present application may be prepared based on the Preparation Examples to be described below.

Another exemplary embodiment of the present application provides an organic light emitting device comprising the heterocyclic compound represented by Chemical Formula 2. The ‘organic light emitting device” may be expressed by terms such as “organic light emitting diode”, “organic light emitting diodes (OLEDs)”, “OLED device”, and “organic electroluminescence device”.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

The organic light emitting device of the present invention may further comprise one or two or more layers selected from the group consisting of a light emitting layer, a hole injection layer, a hole transport layer, an electron injection layer, an electron transport layer, a hole auxiliary layer, and a hole blocking layer.

Furthermore, another exemplary embodiment of the present application provides a composition for an organic material layer of an organic light emitting device, which comprises both the heterocyclic compound represented by Chemical Formula 1 and the compound represented by Chemical Formula 2.

The specific contents on the heterocyclic compound represented by Chemical Formula 1 and the heterocyclic compound represented by Chemical Formula 2 are the same as those described above.

The weight ratio of the heterocyclic compound represented by Chemical Formula 1: the heterocyclic compound represented by Chemical Formula 2 in the composition may be 1:10 to 10:1, 1:8 to 8:1, 1:5 to 5:1, and 1:2 to 2:1, but is not limited thereto.

In an exemplary embodiment of the present application, provided is a method for manufacturing an organic light emitting device, the method comprising: preparing a substrate; forming a first electrode on the substrate; forming an organic material layer having one or more layers on the first electrode; and forming a second electrode on the organic material layer, in which the forming of the organic material layer comprises forming the organic material layer having one or more layers by using the composition for an organic material layer according to an exemplary embodiment of the present application.

In an exemplary embodiment of the present application, provided is a method for manufacturing an organic light emitting device, in which the forming of the organic material layer forms the organic material layer by supplying the heterocyclic compound represented by Chemical Formula 1 and the compound represented by Chemical Formula 2 by each individual supply source, and then using a thermal vacuum deposition method.

In an exemplary embodiment of the present application, provided is a method for manufacturing an organic light emitting device, in which the forming of the organic material layer forms the organic material layer pre-mixing the heterocyclic compound represented by Chemical Formula 1 and the compound represented by Chemical Formula 2 by each individual supply source, and using a thermal vacuum deposition method.

The pre-mixing means that before the heterocyclic compound represented by Chemical Formula 1 and the compound represented by Chemical Formula 2 are deposited onto an organic material layer, the materials are first mixed and the mixture is contained in one common container and mixed.

When the materials are deposited by the pre-mixing, the deposition is not conducted several times, so that the uniformity and thin film characteristics of the thin film may be improved, the process procedures may be simplified, the costs may be reduced, and a device in which the efficiency and service life have been improved may be formed.

The pre-mixed material may be referred to as a composition for an organic material layer according to an exemplary embodiment of the present application.

The organic light emitting device according to an exemplary embodiment of the present application may be manufactured by typical manufacturing methods and materials of the organic light emitting device, except that the above-described heterocyclic compound is used to form an organic material layer.

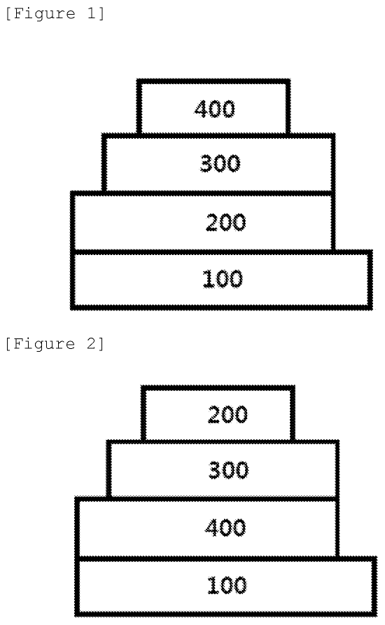

FIGS. 1 to 3 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

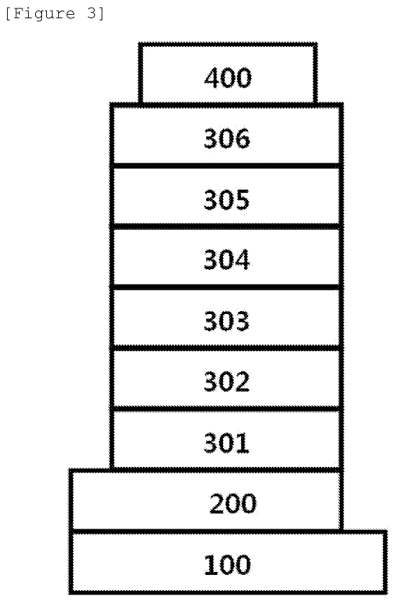

FIG. 3 exemplifies a case where an organic material layer is a multilayer. An organic light emitting device according to FIG. 3 comprises a hole injection layer 301, a hole transporting layer 302, a light emitting layer 303, a hole blocking layer 304, an electron transporting layer 305, and an electron injection layer 306. However, the scope of the present application is not limited by the stacking structure as described above, and if necessary, the other layers except for the light emitting layer may be omitted, and another necessary functional layer may be further added.

In the organic light emitting device according to an exemplary embodiment of the present application, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material comprise: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as Zno:Al or SnO₂:Sb; a conductive polymer such as poly(3-methylthiophene), poly[3,4-(ethylene-1,2-dioxy)thiophene] (PEDOT), polypyrrole, and polyaniline; and the like, but are not limited thereto.

As a material for the negative electrode, materials having a relatively low work function may be used, and a metal, a metal oxide, or a conductive polymer, and the like may be used. Specific examples of the negative electrode material comprise: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto. As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino] triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transporting material, a pyrazoline derivative, an arylamine-based derivative, a stilbene derivative, a triphenyldiamine derivative, and the like may be used, and a low-molecular weight or polymer material may also be used.

As an electron transporting material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. Further, as the light emitting material, a fluorescent material may also be used, but a phosphorescent material may also be used. As the light emitting material, it is also possible to use alone a material which emits light by combining holes and electrons each injected from a positive electrode and a negative electrode, but materials in which a host material and a dopant material are involved in light emission together may also be used.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices comprising organic solar cells, organic photoconductors, organic transistors, and the like, based on the principle similar to those applied to organic light emitting devices.

Hereinafter, the present specification will be described in more detail through Examples, but these Examples are provided only for exemplifying the present application, and are not intended to limit the scope of the present application.

### Preparation Examples

### Preparation Example 1> Preparation of Compound 1-1

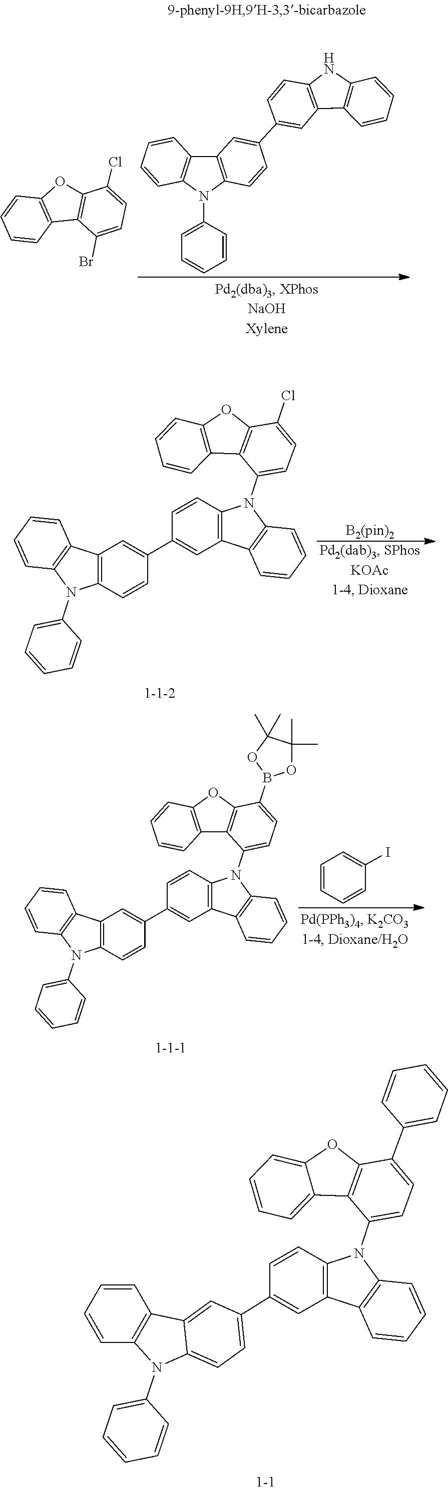

### 1) Preparation of Compound 1-1-2

After 10 g of (35.5 mM) of 1-bromo-4-chlorodibenzo[b, d]furan, 14.5 g (35.5 mM) of 9-phenyl-9H, 9′ H-3,3′-bicarbazole, 3.2 g (3.5 mm) of Pdz (dba)₃, 5.0 g (10.5 mM) of XPhos and 3.5 g (88.7 mM) of NaOH were dissolved in 100 mL of xylene, the resulting solution was refluxed for 12 hours. After the reaction was completed, distilled water and dichloromethane (DCM) were added thereto at room temperature, extraction was performed, the organic layer was dried over MgSO₄, and then the solvent was removed by a rotary evaporator. The reactant was purified by column chromatography (DCM:Hex=1:2) to obtain 11.0 g (50%) of Target Compound 1-1-2.

Dichloromethane is also referred to as methylene chloride, and is also hereinafter referred to as MC.

### 2) Preparation of Compound 1-1-1

After 11.0 g (18.0 mM) of Compound 1-1-2, 6.8 g (27 mM) of bis(pinacolato)diboron), 1.6 g (1.8 mM) of Pd₂(dba)₃, 2.2 g (5.4 mM) of SPhos and 4.4 g (45 mM) of KOAc were dissolved in 100 mL of 1,4-dioxane, the resulting solution was refluxed for 12 hours. After the reaction was completed, distilled water and DCM were added thereto at room temperature, extraction was performed, the organic layer was dried over MgSO₄, and then the solvent was removed by a rotary evaporator. The reactant was purified by column chromatography (DCM:Hex=1:2) to obtain 9.9 g (78%) of Target Compound 1-1-1.

Hex means hexane and is also hereinafter referred to as HX.

### 3) Preparation of Compound 1-1

After 9.9 g (14.1 mM) of Compound 1-1-1, 4.3 g (21.15 mM) of iodobenzene, 0.81 g (0.70 mM) of Pd(PPh₃)₄, and 5.8 g (42.3 mM) of K₂CO₃ were dissolved in 100/20 ml of 1,4-dioxane/H₂O, the resulting solution was refluxed for 4 hours. After the reaction was completed, distilled water and DCM were added thereto at room temperature, extraction was performed, the organic layer was dried over MgSO₄, and then the solvent was removed by a rotary evaporator. The reactant was purified by column chromatography (DCM:Hex=1:3) and recrystallized with methanol to obtain 8.4 g (91%) of Target Compound 1-1.

A target compound was synthesized by performing preparation in the same manner as in Preparation Example 1, except that Intermediate A in the following Table 1 was used instead of 1-bromo-4-chlorodibenzo[b, d]furan, Intermediate B in the following Table 1 was used instead of 9-phenyl-9H, 9′H-3,3′-bicarbazole, and Intermediate C in the following Table 1 was used instead of iodobenzene in Preparation Example 1.

TABLE 1

| Com- | | | | | |
|---|---|---|---|---|---|
| pound | Intermediate | Intermediate | Intermediate | | |
| No. | A | B | C | Target compound | Yield |
| 1-2 | 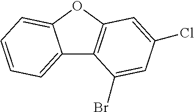 [SMILES: Clc1cc(Br)c2c(c1)oc1ccccc12] | 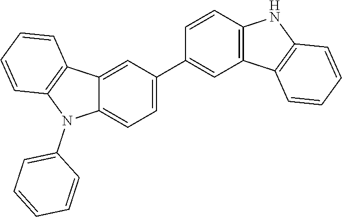 [SMILES: c1ccc(-n2c3ccccc3c3cc(-c4ccc5[nH]c6ccccc6c5c4)ccc32)cc1] | 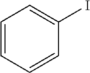 | 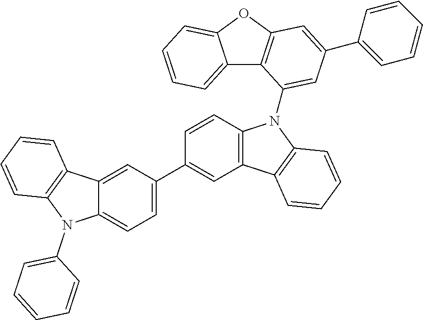 [SMILES: c1ccc(-c2cc(-n3c4ccccc4c4cc(-c5ccc6c(c5)c5ccccc5n6-c5ccccc5)ccc43)c3c(c2)oc2ccccc23)cc1] | 80% |
| 1-4 | 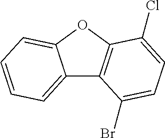 [SMILES: Clc1ccc(Br)c2c1oc1ccccc12] | | 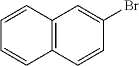 [SMILES: Brc1ccc2ccccc2c1] | 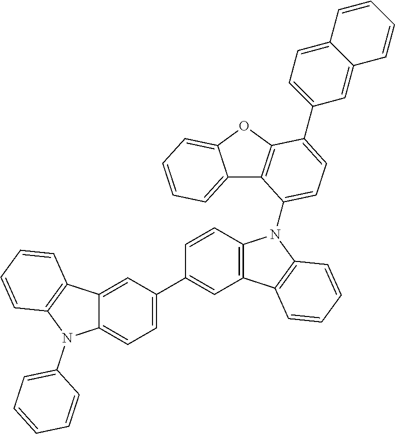 [SMILES: c1ccc(-n2c3ccccc3c3cc(-c4ccc5c(c4)c4ccccc4n5-c4ccc(-c5ccc6ccccc6c5)c5oc6ccccc6c45)ccc32)cc1] | 78% |
| 1-7 | | | 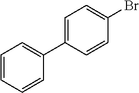 [SMILES: Brc1ccc(-c2ccccc2)cc1] | 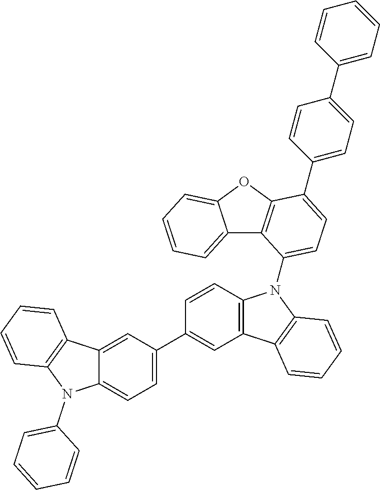 [SMILES: c1ccc(-c2ccc(-c3ccc(-n4c5ccccc5c5cc(-c6ccc7c(c6)c6ccccc6n7-c6ccccc6)ccc54)c4c3oc3ccccc34)cc2)cc1] | 83% |
| 1-8 | | | 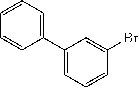 [SMILES: Brc1cccc(-c2ccccc2)c1] | 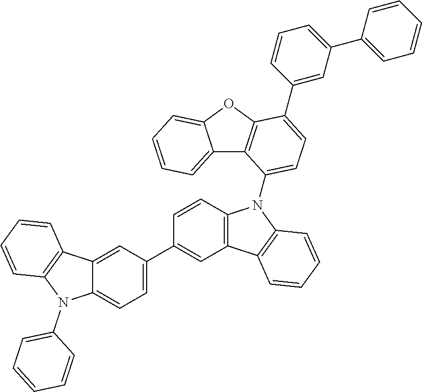 [SMILES: c1ccc(-c2cccc(-c3ccc(-n4c5ccccc5c5cc(-c6ccc7c(c6)c6ccccc6n7-c6ccccc6)ccc54)c4c3oc3ccccc34)c2)cc1] | 69% |
| 1-12 | 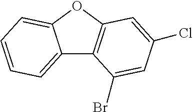 [SMILES: Clc1cc(Br)c2c(c1)oc1ccccc12] | | 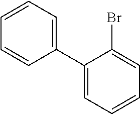 [SMILES: Brc1ccccc1-c1ccccc1] | 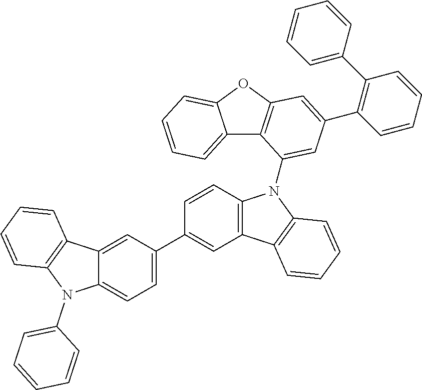 [SMILES: c1ccc(-c2ccccc2-c2cc(-n3c4ccccc4c4cc(-c5ccc6c(c5)c5ccccc5n6-c5ccccc5)ccc43)c3c(c2)oc2ccccc23)cc1] | 72% |
| 1-13 | 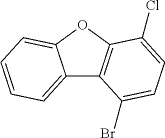 [SMILES: Clc1ccc(Br)c2c1oc1ccccc12] | 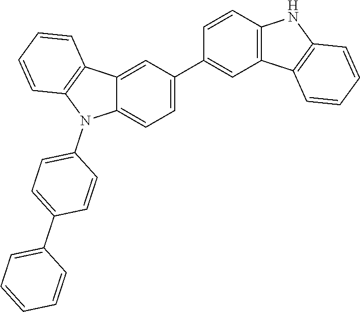 [SMILES: c1ccc(-c2ccc(-n3c4ccccc4c4cc(-c5ccc6[nH]c7ccccc7c6c5)ccc43)cc2)cc1] | 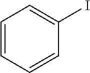 | 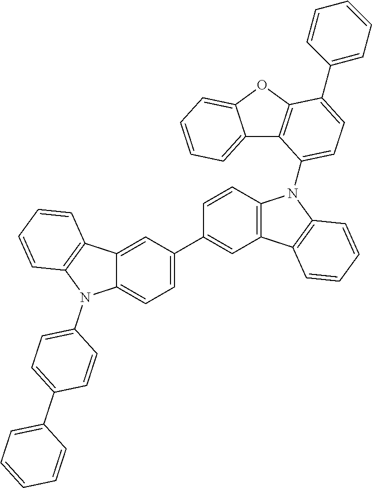 [SMILES: c1ccc(-c2ccc(-n3c4ccccc4c4cc(-c5ccc6c(c5)c5ccccc5n6-c5ccc(-c6ccccc6)c6oc7ccccc7c56)ccc43)cc2)cc1] | 80% |
| 1-15 | | 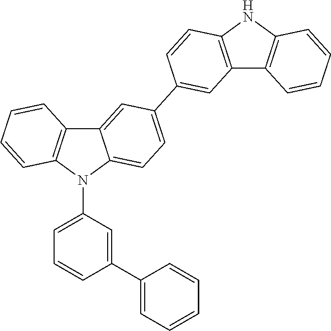 [SMILES: c1ccc(-c2cccc(-n3c4ccccc4c4cc(-c5ccc6[nH]c7ccccc7c6c5)ccc43)c2)cc1] | | 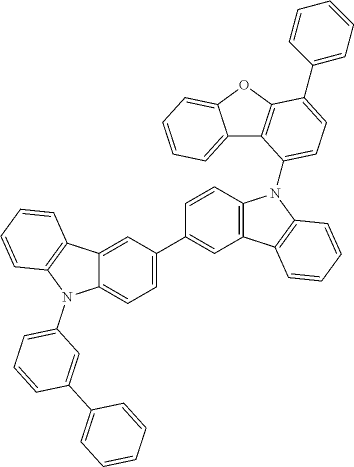 [SMILES: c1ccc(-c2cccc(-n3c4ccccc4c4cc(-c5ccc6c(c5)c5ccccc5n6-c5ccc(-c6ccccc6)c6oc7ccccc7c56)ccc43)c2)cc1] | 81% |
| 1-17 | | 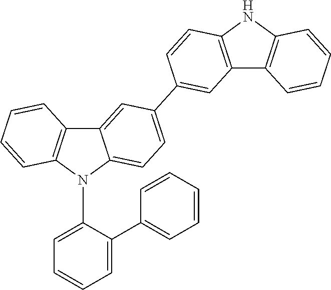 [SMILES: c1ccc(-c2ccccc2-n2c3ccccc3c3cc(-c4ccc5[nH]c6ccccc6c5c4)ccc32)cc1] | | 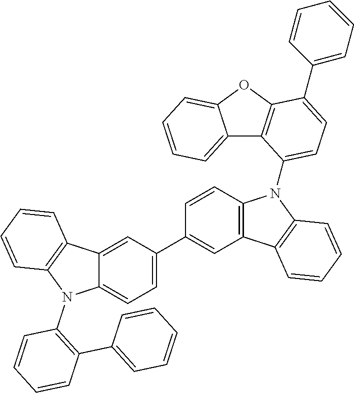 [SMILES: c1ccc(-c2ccccc2-n2c3ccccc3c3cc(-c4ccc5c(c4)c4ccccc4n5-c4ccc(-c5ccccc5)c5oc6ccccc6c45)ccc32)cc1] | 81% |
| 1-18 | 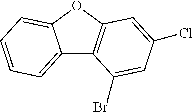 [SMILES: Clc1cc(Br)c2c(c1)oc1ccccc12] | | | 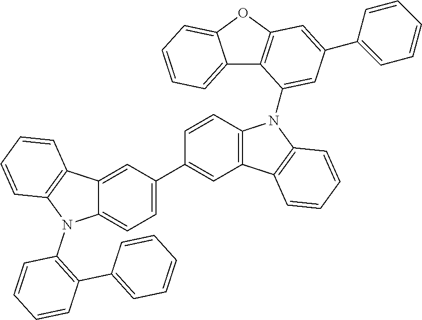 [SMILES: c1ccc(-c2cc(-n3c4ccccc4c4cc(-c5ccc6c(c5)c5ccccc5n6-c5ccccc5-c5ccccc5)ccc43)c3c(c2)oc2ccccc23)cc1] | 77% |
| 1-25 | 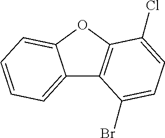 [SMILES: Clc1ccc(Br)c2c1oc1ccccc12] | 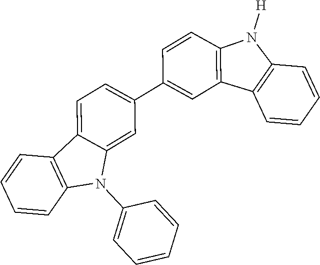 [SMILES: c1ccc(-n2c3ccccc3c3ccc(-c4ccc5[nH]c6ccccc6c5c4)cc32)cc1] | | 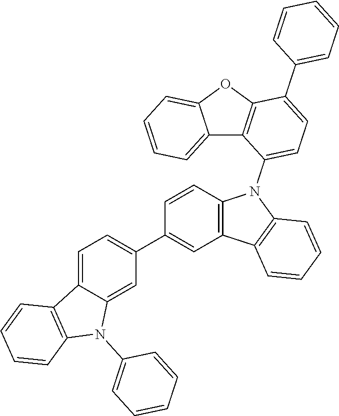 [SMILES: c1ccc(-c2ccc(-n3c4ccccc4c4cc(-c5ccc6c7ccccc7n(-c7ccccc7)c6c5)ccc43)c3c2oc2ccccc23)cc1] | 76% |
| 1-27 | | | 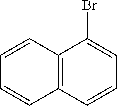 [SMILES: Brc1cccc2ccccc12] | 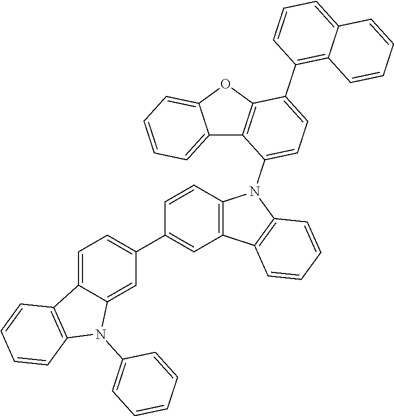 [SMILES: c1ccc(-n2c3ccccc3c3ccc(-c4ccc5c(c4)c4ccccc4n5-c4ccc(-c5cccc6ccccc56)c5oc6ccccc6c45)cc32)cc1] | 80% |
| 1-30 | 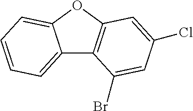 [SMILES: Clc1cc(Br)c2c(c1)oc1ccccc12] | | 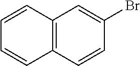 [SMILES: Brc1ccc2ccccc2c1] | 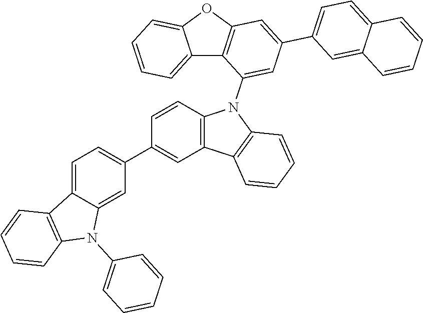 [SMILES: c1ccc(-n2c3ccccc3c3ccc(-c4ccc5c(c4)c4ccccc4n5-c4cc(-c5ccc6ccccc6c5)cc5oc6ccccc6c45)cc32)cc1] | 82% |
| 1-32 | 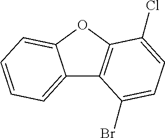 [SMILES: Clc1ccc(Br)c2c1oc1ccccc12] | | 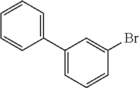 [SMILES: Brc1cccc(-c2ccccc2)c1] | 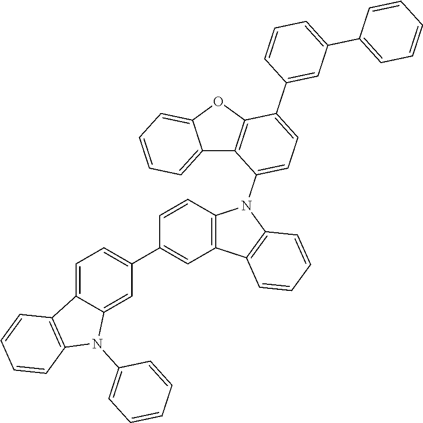 [SMILES: c1ccc(-c2cccc(-c3ccc(-n4c5ccccc5c5cc(-c6ccc7c8ccccc8n(-c8ccccc8)c7c6)ccc54)c4c3oc3ccccc34)c2)cc1] | 81% |
| 1-34 | 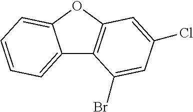 [SMILES: Clc1cc(Br)c2c(c1)oc1ccccc12] | | 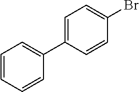 [SMILES: Brc1ccc(-c2ccccc2)cc1] | 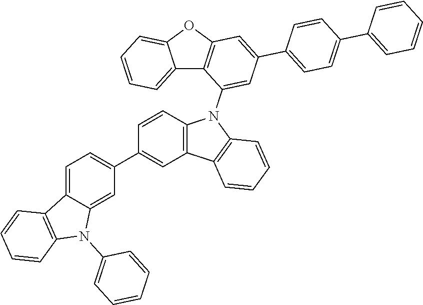 [SMILES: c1ccc(-c2ccc(-c3cc(-n4c5ccccc5c5cc(-c6ccc7c8ccccc8n(-c8ccccc8)c7c6)ccc54)c4c(c3)oc3ccccc34)cc2)cc1] | 80% |
| 1-37 | 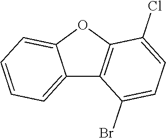 [SMILES: Clc1ccc(Br)c2c1oc1ccccc12] | 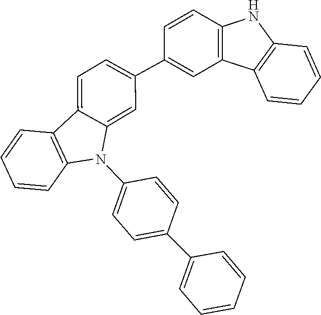 [SMILES: c1ccc(-c2ccc(-n3c4ccccc4c4ccc(-c5ccc6[nH]c7ccccc7c6c5)cc43)cc2)cc1] | 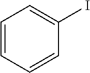 | 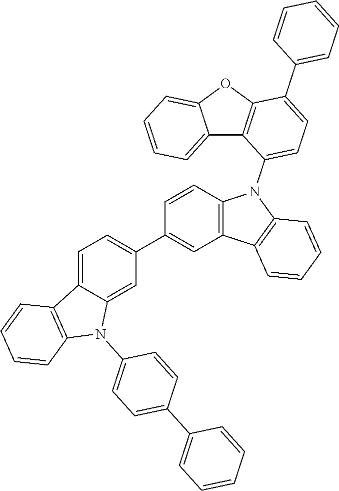 [SMILES: c1ccc(-c2ccc(-n3c4ccccc4c4ccc(-c5ccc6c(c5)c5ccccc5n6-c5ccc(-c6ccccc6)c6oc7ccccc7c56)cc43)cc2)cc1] | 91% |
| 1-38 | 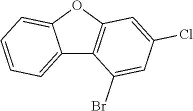 [SMILES: Clc1cc(Br)c2c(c1)oc1ccccc12] | | | 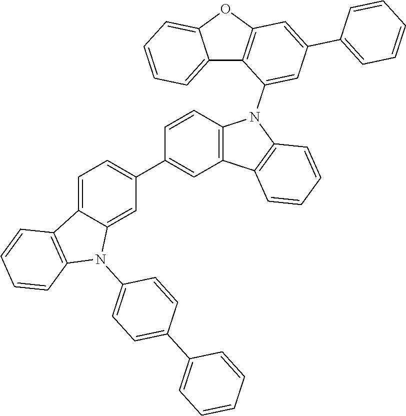 [SMILES: c1ccc(-c2ccc(-n3c4ccccc4c4ccc(-c5ccc6c(c5)c5ccccc5n6-c5cc(-c6ccccc6)cc6oc7ccccc7c56)cc43)cc2)cc1] | 85% |
| 1-39 | 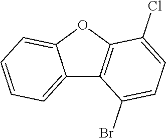 [SMILES: Clc1ccc(Br)c2c1oc1ccccc12] | 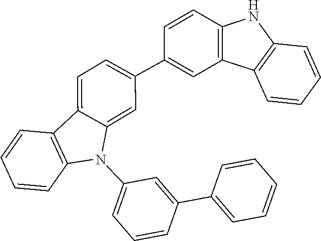 [SMILES: c1ccc(-c2cccc(-n3c4ccccc4c4ccc(-c5ccc6[nH]c7ccccc7c6c5)cc43)c2)cc1] | | 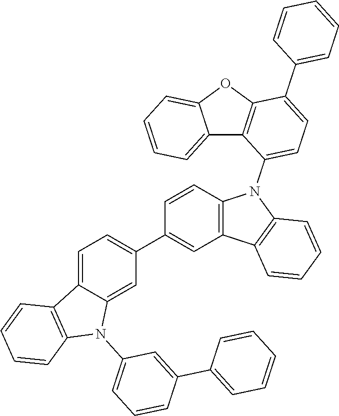 [SMILES: c1ccc(-c2cccc(-n3c4ccccc4c4ccc(-c5ccc6c(c5)c5ccccc5n6-c5ccc(-c6ccccc6)c6oc7ccccc7c56)cc43)c2)cc1] | 82% |
| 1-40 | 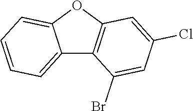 [SMILES: Clc1cc(Br)c2c(c1)oc1ccccc12] | | | 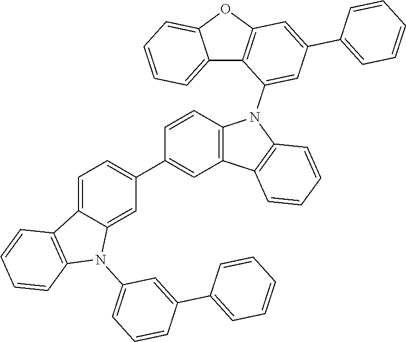 [SMILES: c1ccc(-c2cccc(-n3c4ccccc4c4ccc(-c5ccc6c(c5)c5ccccc5n6-c5cc(-c6ccccc6)cc6oc7ccccc7c56)cc43)c2)cc1] | 82% |
| 1-41 | 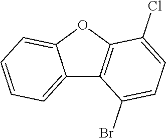 [SMILES: Clc1ccc(Br)c2c1oc1ccccc12] | 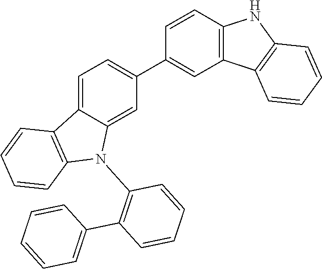 [SMILES: c1ccc(-c2ccccc2-n2c3ccccc3c3ccc(-c4ccc5[nH]c6ccccc6c5c4)cc32)cc1] | | 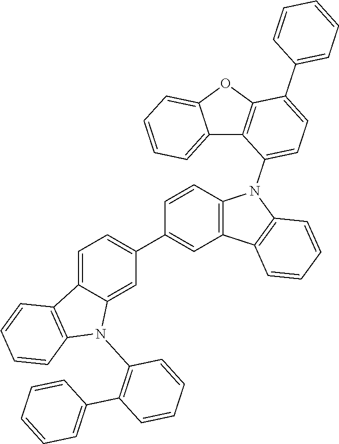 [SMILES: c1ccc(-c2ccccc2-n2c3ccccc3c3ccc(-c4ccc5c(c4)c4ccccc4n5-c4ccc(-c5ccccc5)c5oc6ccccc6c45)cc32)cc1] | 76% |
| 1-49 | | 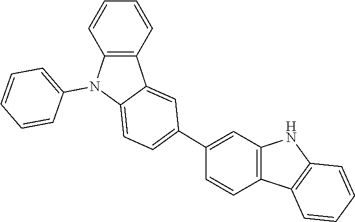 [SMILES: c1ccc(-n2c3ccccc3c3cc(-c4ccc5c(c4)[nH]c4ccccc45)ccc32)cc1] | | 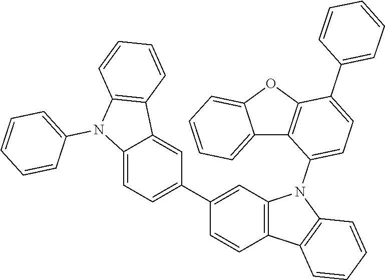 [SMILES: c1ccc(-c2ccc(-n3c4ccccc4c4ccc(-c5ccc6c(c5)c5ccccc5n6-c5ccccc5)cc43)c3c2oc2ccccc23)cc1] | 77% |
| 1-50 | 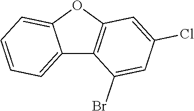 [SMILES: Clc1cc(Br)c2c(c1)oc1ccccc12] | | | 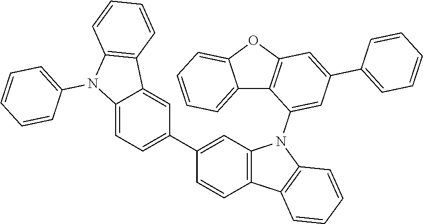 [SMILES: c1ccc(-c2cc(-n3c4ccccc4c4ccc(-c5ccc6c(c5)c5ccccc5n6-c5ccccc5)cc43)c3c(c2)oc2ccccc23)cc1] | 90% |
| 1-51 | 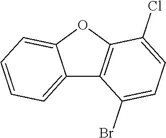 [SMILES: Clc1ccc(Br)c2c1oc1ccccc12] | | 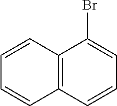 [SMILES: Brc1cccc2ccccc12] | 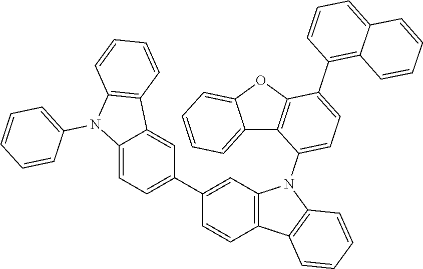 [SMILES: c1ccc(-n2c3ccccc3c3cc(-c4ccc5c6ccccc6n(-c6ccc(-c7cccc8ccccc78)c7oc8ccccc8c67)c5c4)ccc32)cc1] | 81% |
| 1-54 | 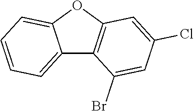 [SMILES: Clc1cc(Br)c2c(c1)oc1ccccc12] | | 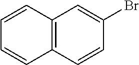 [SMILES: Brc1ccc2ccccc2c1] | 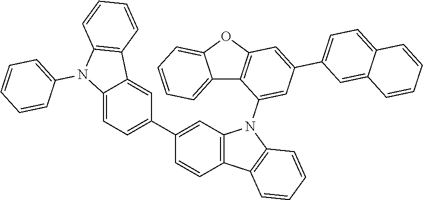 [SMILES: c1ccc(-n2c3ccccc3c3cc(-c4ccc5c6ccccc6n(-c6cc(-c7ccc8ccccc8c7)cc7oc8ccccc8c67)c5c4)ccc32)cc1] | 77% |
| 1-56 | 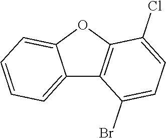 [SMILES: Clc1ccc(Br)c2c1oc1ccccc12] | | 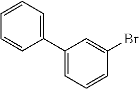 [SMILES: Brc1cccc(-c2ccccc2)c1] | 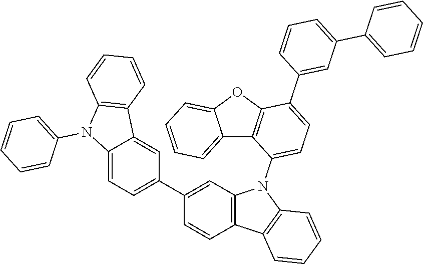 [SMILES: c1ccc(-c2cccc(-c3ccc(-n4c5ccccc5c5ccc(-c6ccc7c(c6)c6ccccc6n7-c6ccccc6)cc54)c4c3oc3ccccc34)c2)cc1] | 89% |
| 1-57 | | | 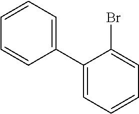 [SMILES: Brc1ccccc1-c1ccccc1] | 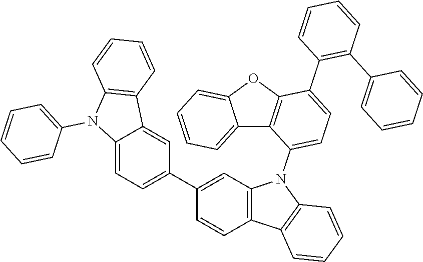 [SMILES: c1ccc(-c2ccccc2-c2ccc(-n3c4ccccc4c4ccc(-c5ccc6c(c5)c5ccccc5n6-c5ccccc5)cc43)c3c2oc2ccccc23)cc1] | 88% |
| 1-59 | 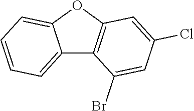 [SMILES: Clc1cc(Br)c2c(c1)oc1ccccc12] | | 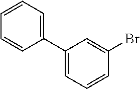 [SMILES: Brc1cccc(-c2ccccc2)c1] | 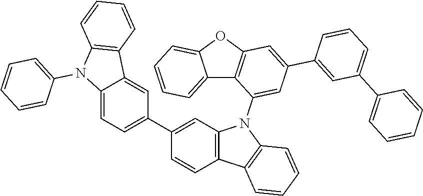 [SMILES: c1ccc(-c2cccc(-c3cc(-n4c5ccccc5c5ccc(-c6ccc7c(c6)c6ccccc6n7-c6ccccc6)cc54)c4c(c3)oc3ccccc34)c2)cc1] | 89% |
| 1-61 | 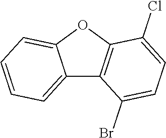 [SMILES: Clc1ccc(Br)c2c1oc1ccccc12] | 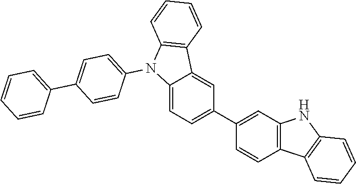 [SMILES: c1ccc(-c2ccc(-n3c4ccccc4c4cc(-c5ccc6c(c5)[nH]c5ccccc56)ccc43)cc2)cc1] | 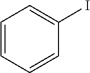 | 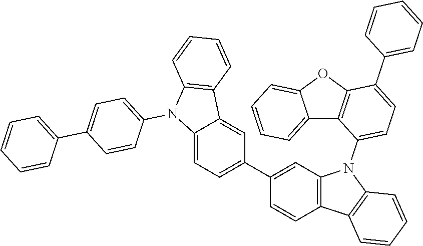 [SMILES: c1ccc(-c2ccc(-n3c4ccccc4c4cc(-c5ccc6c7ccccc7n(-c7ccc(-c8ccccc8)c8oc9ccccc9c78)c6c5)ccc43)cc2)cc1] | 78% |
| 1-64 | | 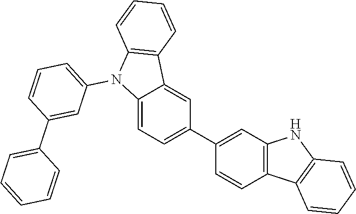 [SMILES: c1ccc(-c2cccc(-n3c4ccccc4c4cc(-c5ccc6c(c5)[nH]c5ccccc56)ccc43)c2)cc1] | | 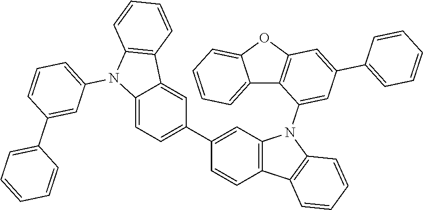 [SMILES: c1ccc(-c2cccc(-n3c4ccccc4c4cc(-c5ccc6c7ccccc7n(-c7cc(-c8ccccc8)cc8oc9ccccc9c78)c6c5)ccc43)c2)cc1] | 70% |
| 1-66 | 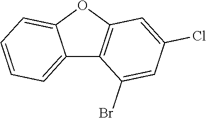 [SMILES: Clc1cc(Br)c2c(c1)oc1ccccc12] | 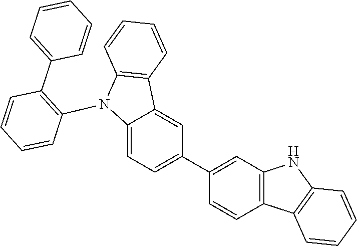 [SMILES: c1ccc(-c2ccccc2-n2c3ccccc3c3cc(-c4ccc5c(c4)[nH]c4ccccc45)ccc32)cc1] | | 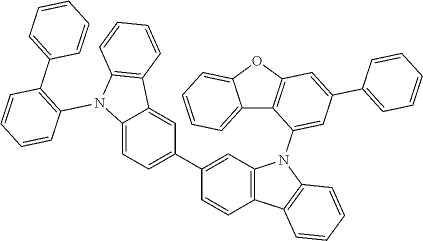 [SMILES: c1ccc(-c2cc(-n3c4ccccc4c4ccc(-c5ccc6c(c5)c5ccccc5n6-c5ccccc5-c5ccccc5)cc43)c3c(c2)oc2ccccc23)cc1] | 81% |
| 1-73 | 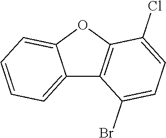 [SMILES: Clc1ccc(Br)c2c1oc1ccccc12] | 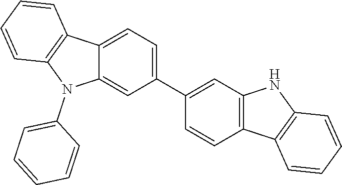 [SMILES: c1ccc(-n2c3ccccc3c3ccc(-c4ccc5c(c4)[nH]c4ccccc45)cc32)cc1] | | 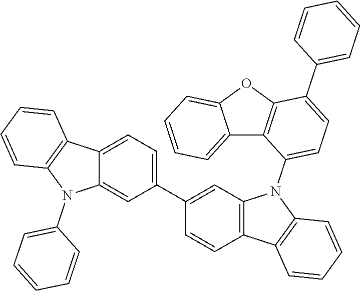 [SMILES: c1ccc(-c2ccc(-n3c4ccccc4c4ccc(-c5ccc6c7ccccc7n(-c7ccccc7)c6c5)cc43)c3c2oc2ccccc23)cc1] | 90% |
| 1-74 | 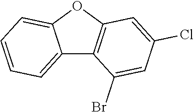 [SMILES: Clc1cc(Br)c2c(c1)oc1ccccc12] | | | 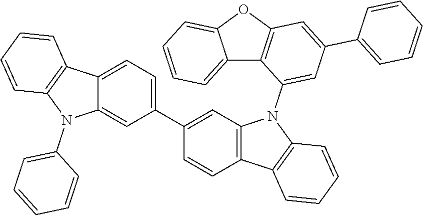 [SMILES: c1ccc(-c2cc(-n3c4ccccc4c4ccc(-c5ccc6c7ccccc7n(-c7ccccc7)c6c5)cc43)c3c(c2)oc2ccccc23)cc1] | 78% |
| 1-78 | | | 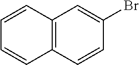 [SMILES: Brc1ccc2ccccc2c1] | 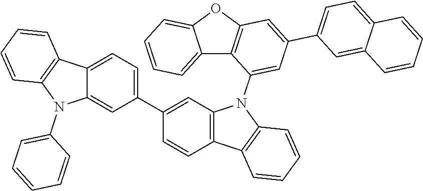 [SMILES: c1ccc(-n2c3ccccc3c3ccc(-c4ccc5c6ccccc6n(-c6cc(-c7ccc8ccccc8c7)cc7oc8ccccc8c67)c5c4)cc32)cc1] | 79% |
| 1-80 | 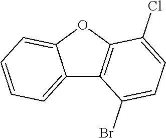 [SMILES: Clc1ccc(Br)c2c1oc1ccccc12] | | 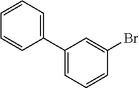 [SMILES: Brc1cccc(-c2ccccc2)c1] | 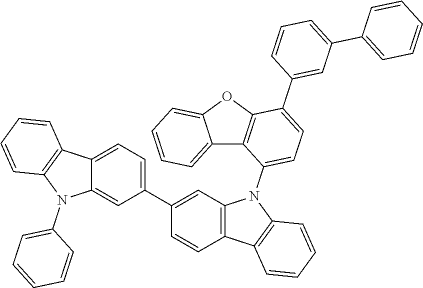 [SMILES: c1ccc(-c2cccc(-c3ccc(-n4c5ccccc5c5ccc(-c6ccc7c8ccccc8n(-c8ccccc8)c7c6)cc54)c4c3oc3ccccc34)c2)cc1] | 77% |
| 1-81 | | | 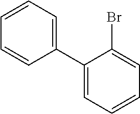 [SMILES: Brc1ccccc1-c1ccccc1] | 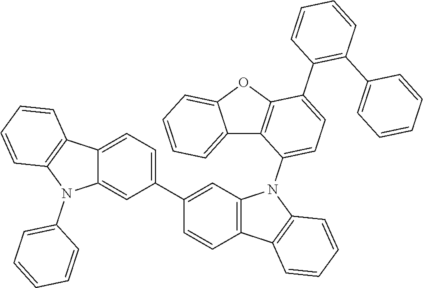 [SMILES: c1ccc(-c2ccccc2-c2ccc(-n3c4ccccc4c4ccc(-c5ccc6c7ccccc7n(-c7ccccc7)c6c5)cc43)c3c2oc2ccccc23)cc1] | 83% |
| 1-83 | 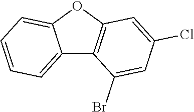 [SMILES: Clc1cc(Br)c2c(c1)oc1ccccc12] | | 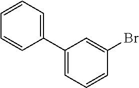 [SMILES: Brc1cccc(-c2ccccc2)c1] | 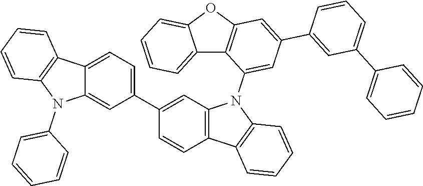 [SMILES: c1ccc(-c2cccc(-c3cc(-n4c5ccccc5c5ccc(-c6ccc7c8ccccc8n(-c8ccccc8)c7c6)cc54)c4c(c3)oc3ccccc34)c2)cc1] | 88% |
| 1-85 | 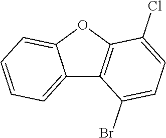 [SMILES: Clc1ccc(Br)c2c1oc1ccccc12] | 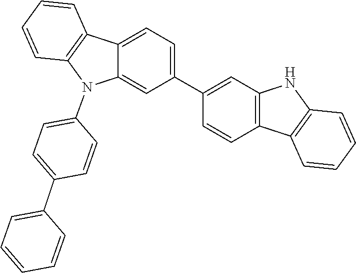 [SMILES: c1ccc(-c2ccc(-n3c4ccccc4c4ccc(-c5ccc6c(c5)[nH]c5ccccc56)cc43)cc2)cc1] | 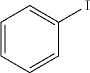 | 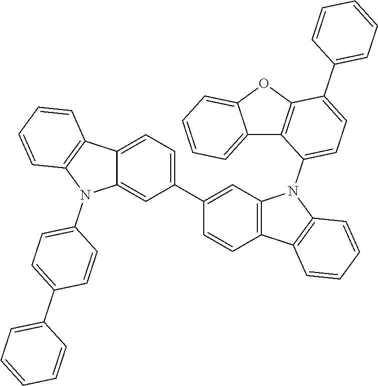 [SMILES: c1ccc(-c2ccc(-n3c4ccccc4c4ccc(-c5ccc6c7ccccc7n(-c7ccc(-c8ccccc8)c8oc9ccccc9c78)c6c5)cc43)cc2)cc1] | 88% |
| 1-88 | 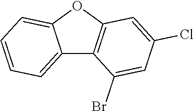 [SMILES: Clc1cc(Br)c2c(c1)oc1ccccc12] | 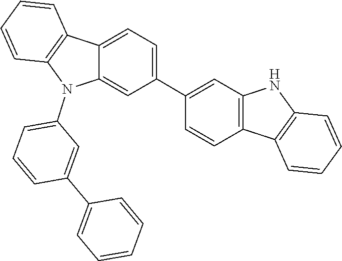 [SMILES: c1ccc(-c2cccc(-n3c4ccccc4c4ccc(-c5ccc6c(c5)[nH]c5ccccc56)cc43)c2)cc1] | | 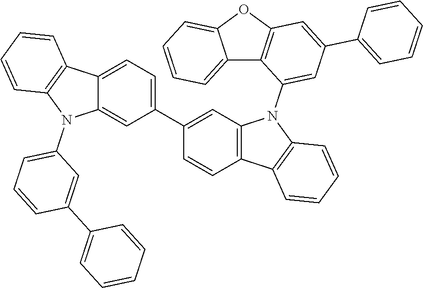 [SMILES: c1ccc(-c2cccc(-n3c4ccccc4c4ccc(-c5ccc6c7ccccc7n(-c7cc(-c8ccccc8)cc8oc9ccccc9c78)c6c5)cc43)c2)cc1] | 79% |
| 1-90 | | 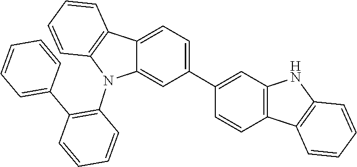 [SMILES: c1ccc(-c2ccccc2-n2c3ccccc3c3ccc(-c4ccc5c(c4)[nH]c4ccccc45)cc32)cc1] | | 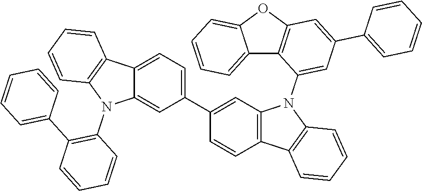 [SMILES: c1ccc(-c2cc(-n3c4ccccc4c4ccc(-c5ccc6c7ccccc7n(-c7ccccc7-c7ccccc7)c6c5)cc43)c3c(c2)oc2ccccc23)cc1] | 78% |
| 1-97 | 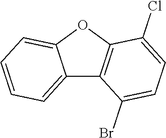 [SMILES: Clc1ccc(Br)c2c1oc1ccccc12] | 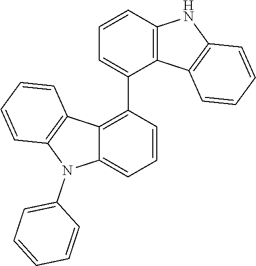 [SMILES: c1ccc(-n2c3ccccc3c3c(-c4cccc5[nH]c6ccccc6c45)cccc32)cc1] | | 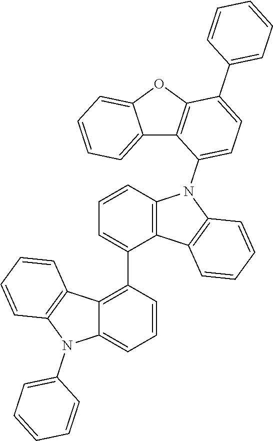 [SMILES: c1ccc(-c2ccc(-n3c4ccccc4c4c(-c5cccc6c5c5ccccc5n6-c5ccccc5)cccc43)c3c2oc2ccccc23)cc1] | 90% |
| 1-98 | 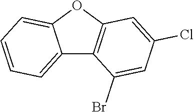 [SMILES: Clc1cc(Br)c2c(c1)oc1ccccc12] | | | 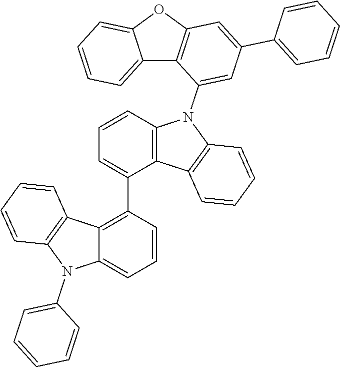 [SMILES: c1ccc(-c2cc(-n3c4ccccc4c4c(-c5cccc6c5c5ccccc5n6-c5ccccc5)cccc43)c3c(c2)oc2ccccc23)cc1] | 91% |
| 1-102 | | | 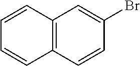 [SMILES: Brc1ccc2ccccc2c1] | 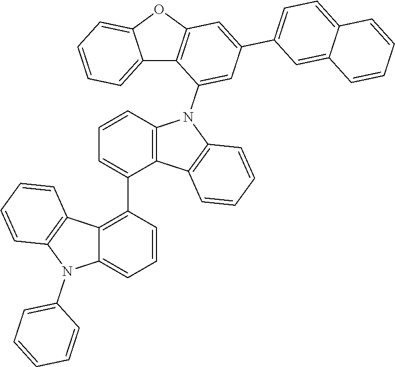 [SMILES: c1ccc(-n2c3ccccc3c3c(-c4cccc5c4c4ccccc4n5-c4cc(-c5ccc6ccccc6c5)cc5oc6ccccc6c45)cccc32)cc1] | 88% |
| 1-107 | | | 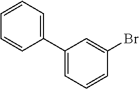 [SMILES: Brc1cccc(-c2ccccc2)c1] | 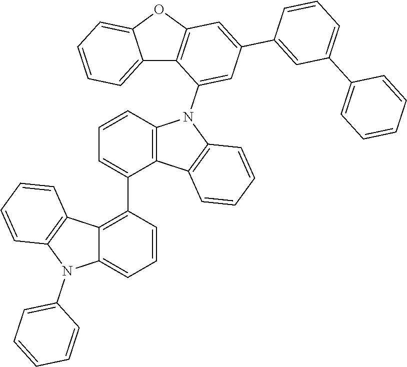 [SMILES: c1ccc(-c2cccc(-c3cc(-n4c5ccccc5c5c(-c6cccc7c6c6ccccc6n7-c6ccccc6)cccc54)c4c(c3)oc3ccccc34)c2)cc1] | 86% |
| 1-109 | 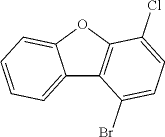 [SMILES: Clc1ccc(Br)c2c1oc1ccccc12] | 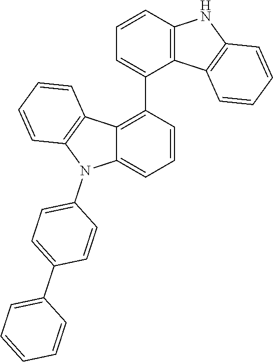 [SMILES: c1ccc(-c2ccc(-n3c4ccccc4c4c(-c5cccc6[nH]c7ccccc7c56)cccc43)cc2)cc1] | 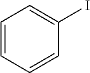 | 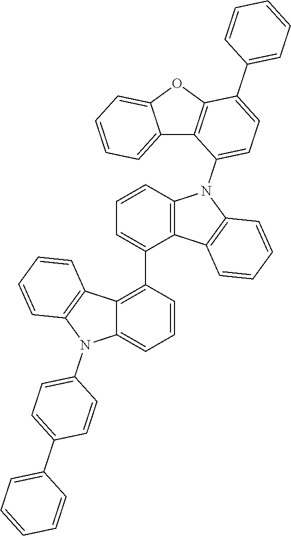 [SMILES: c1ccc(-c2ccc(-n3c4ccccc4c4c(-c5cccc6c5c5ccccc5n6-c5ccc(-c6ccccc6)c6oc7ccccc7c56)cccc43)cc2)cc1] | 79% |
| 1-111 | | 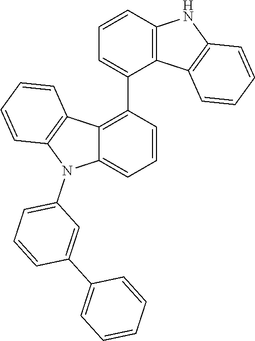 [SMILES: c1ccc(-c2cccc(-n3c4ccccc4c4c(-c5cccc6[nH]c7ccccc7c56)cccc43)c2)cc1] | | 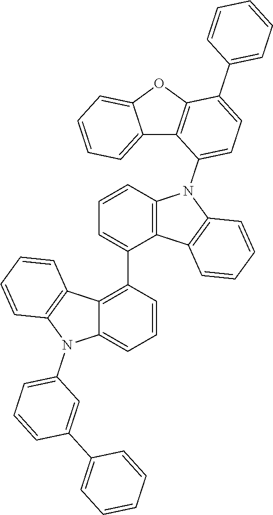 [SMILES: c1ccc(-c2cccc(-n3c4ccccc4c4c(-c5cccc6c5c5ccccc5n6-c5ccc(-c6ccccc6)c6oc7ccccc7c56)cccc43)c2)cc1] | 81% |
| 1-112 | 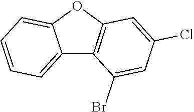 [SMILES: Clc1cc(Br)c2c(c1)oc1ccccc12] | | | 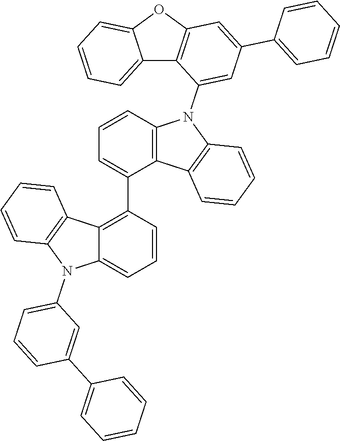 [SMILES: c1ccc(-c2cccc(-n3c4ccccc4c4c(-c5cccc6c5c5ccccc5n6-c5cc(-c6ccccc6)cc6oc7ccccc7c56)cccc43)c2)cc1] | 88% |
| 1-113 | 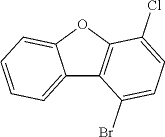 [SMILES: Clc1ccc(Br)c2c1oc1ccccc12] | 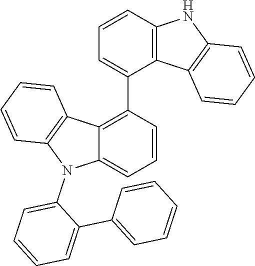 [SMILES: c1ccc(-c2ccccc2-n2c3ccccc3c3c(-c4cccc5[nH]c6ccccc6c45)cccc32)cc1] | | 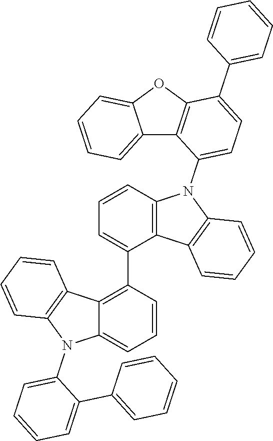 [SMILES: c1ccc(-c2ccccc2-n2c3ccccc3c3c(-c4cccc5c4c4ccccc4n5-c4ccc(-c5ccccc5)c5oc6ccccc6c45)cccc32)cc1] | 91% |
| 1-114 | 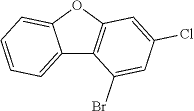 [SMILES: Clc1cc(Br)c2c(c1)oc1ccccc12] | | | 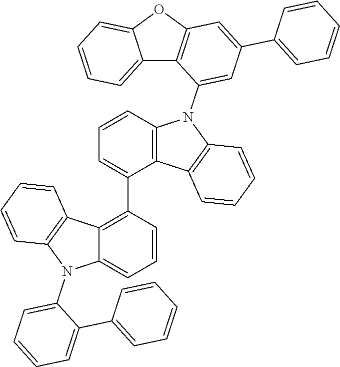 [SMILES: c1ccc(-c2cc(-n3c4ccccc4c4c(-c5cccc6c5c5ccccc5n6-c5ccccc5-c5ccccc5)cccc43)c3c(c2)oc2ccccc23)cc1] | 87% |
| 1-121 | 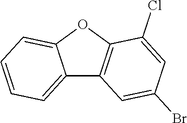 [SMILES: Clc1cc(Br)cc2c1oc1ccccc12] | 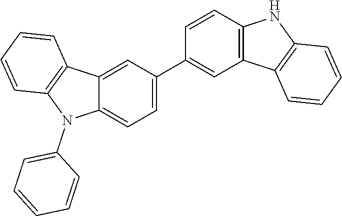 [SMILES: c1ccc(-n2c3ccccc3c3cc(-c4ccc5[nH]c6ccccc6c5c4)ccc32)cc1] | | 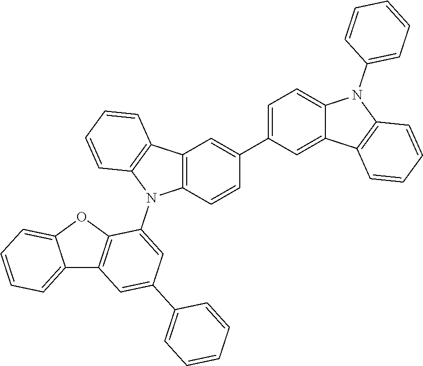 [SMILES: c1ccc(-c2cc(-n3c4ccccc4c4cc(-c5ccc6c(c5)c5ccccc5n6-c5ccccc5)ccc43)c3oc4ccccc4c3c2)cc1] | 88% |
| 1-122 | | | 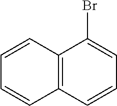 [SMILES: Brc1cccc2ccccc12] | 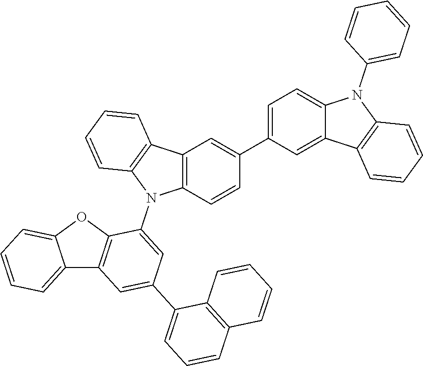 [SMILES: c1ccc(-n2c3ccccc3c3cc(-c4ccc5c(c4)c4ccccc4n5-c4cc(-c5cccc6ccccc56)cc5c4oc4ccccc45)ccc32)cc1] | 79% |
| 1-123 | | | 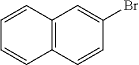 [SMILES: Brc1ccc2ccccc2c1] | 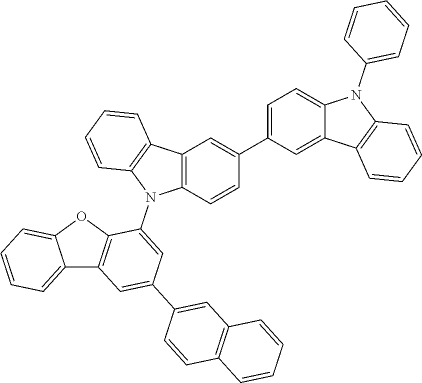 [SMILES: c1ccc(-n2c3ccccc3c3cc(-c4ccc5c(c4)c4ccccc4n5-c4cc(-c5ccc6ccccc6c5)cc5c4oc4ccccc45)ccc32)cc1] | 79% |
| 1-124 | | | 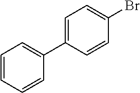 [SMILES: Brc1ccc(-c2ccccc2)cc1] | 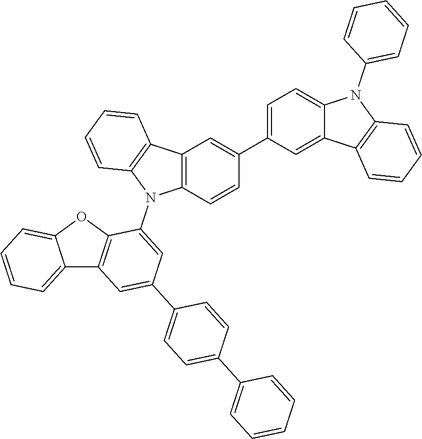 [SMILES: c1ccc(-c2ccc(-c3cc(-n4c5ccccc5c5cc(-c6ccc7c(c6)c6ccccc6n7-c6ccccc6)ccc54)c4oc5ccccc5c4c3)cc2)cc1] | 80% |
| 1-125 | | | 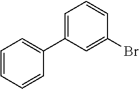 [SMILES: Brc1cccc(-c2ccccc2)c1] | 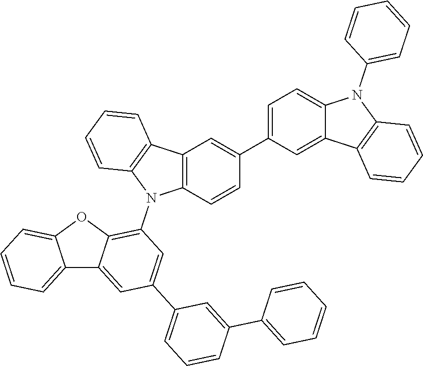 [SMILES: c1ccc(-c2cccc(-c3cc(-n4c5ccccc5c5cc(-c6ccc7c(c6)c6ccccc6n7-c6ccccc6)ccc54)c4oc5ccccc5c4c3)c2)cc1] | 81% |
| 1-127 | | 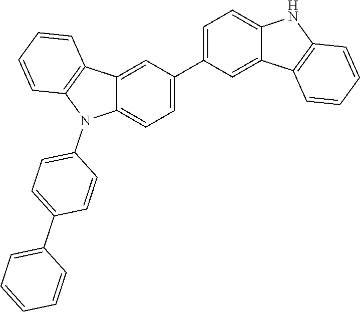 [SMILES: c1ccc(-c2ccc(-n3c4ccccc4c4cc(-c5ccc6[nH]c7ccccc7c6c5)ccc43)cc2)cc1] | 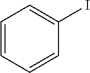 | 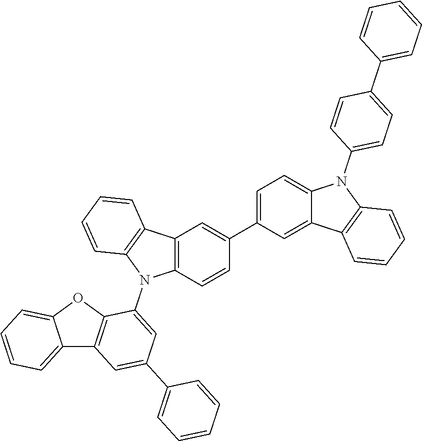 [SMILES: c1ccc(-c2ccc(-n3c4ccccc4c4cc(-c5ccc6c(c5)c5ccccc5n6-c5cc(-c6ccccc6)cc6c5oc5ccccc56)ccc43)cc2)cc1] | 81% |
| 1-128 | | 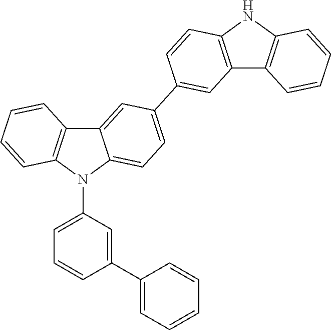 [SMILES: c1ccc(-c2cccc(-n3c4ccccc4c4cc(-c5ccc6[nH]c7ccccc7c6c5)ccc43)c2)cc1] | | 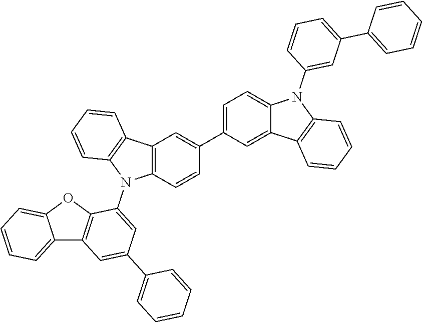 [SMILES: c1ccc(-c2cccc(-n3c4ccccc4c4cc(-c5ccc6c(c5)c5ccccc5n6-c5cc(-c6ccccc6)cc6c5oc5ccccc56)ccc43)c2)cc1] | 77% |
| 1-129 | | 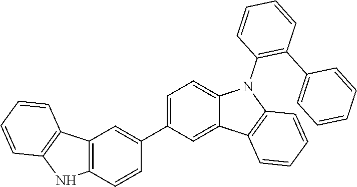 [SMILES: c1ccc(-c2ccccc2-n2c3ccccc3c3cc(-c4ccc5[nH]c6ccccc6c5c4)ccc32)cc1] | | 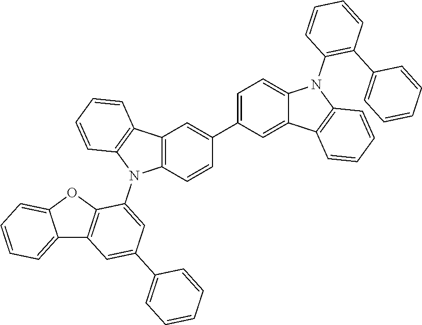 [SMILES: c1ccc(-c2cc(-n3c4ccccc4c4cc(-c5ccc6c(c5)c5ccccc5n6-c5ccccc5-c5ccccc5)ccc43)c3oc4ccccc4c3c2)cc1] | 90% |
| 1-133 | | 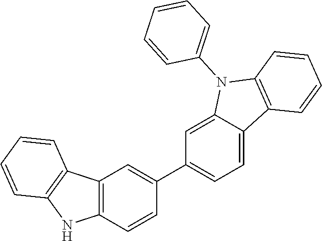 [SMILES: c1ccc(-n2c3ccccc3c3ccc(-c4ccc5[nH]c6ccccc6c5c4)cc32)cc1] | | 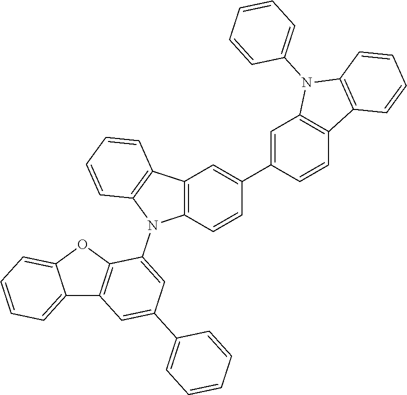 [SMILES: c1ccc(-c2cc(-n3c4ccccc4c4cc(-c5ccc6c7ccccc7n(-c7ccccc7)c6c5)ccc43)c3oc4ccccc4c3c2)cc1] | 90% |
| 1-148 | | 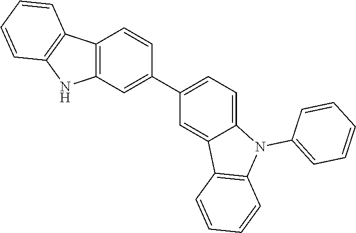 [SMILES: c1ccc(-n2c3ccccc3c3cc(-c4ccc5c(c4)[nH]c4ccccc45)ccc32)cc1] | 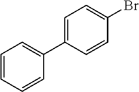 [SMILES: Brc1ccc(-c2ccccc2)cc1] | 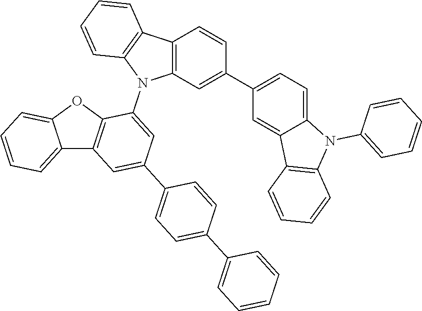 [SMILES: c1ccc(-c2ccc(-c3cc(-n4c5ccccc5c5ccc(-c6ccc7c(c6)c6ccccc6n7-c6ccccc6)cc54)c4oc5ccccc5c4c3)cc2)cc1] | 71% |
| 1-149 | | | 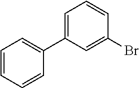 [SMILES: Brc1cccc(-c2ccccc2)c1] | 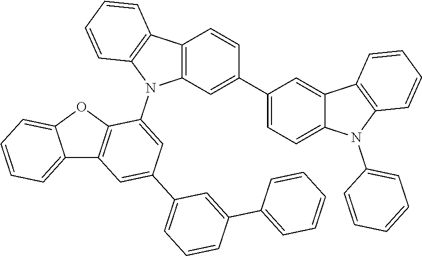 [SMILES: c1ccc(-c2cccc(-c3cc(-n4c5ccccc5c5ccc(-c6ccc7c(c6)c6ccccc6n7-c6ccccc6)cc54)c4oc5ccccc5c4c3)c2)cc1] | 79% |
| 1-150 | | | 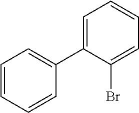 [SMILES: Brc1ccccc1-c1ccccc1] | 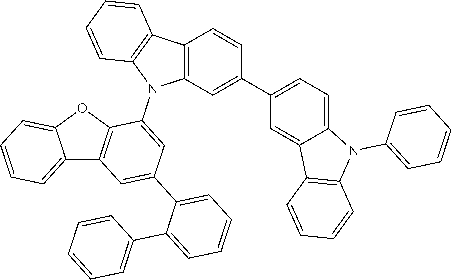 [SMILES: c1ccc(-c2ccccc2-c2cc(-n3c4ccccc4c4ccc(-c5ccc6c(c5)c5ccccc5n6-c5ccccc5)cc43)c3oc4ccccc4c3c2)cc1] | 79% |
| 1-151 | | 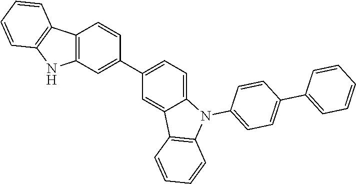 [SMILES: c1ccc(-c2ccc(-n3c4ccccc4c4cc(-c5ccc6c(c5)[nH]c5ccccc56)ccc43)cc2)cc1] | 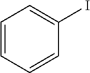 | 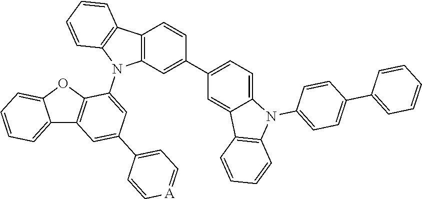 | 76% |
| 1-152 | | 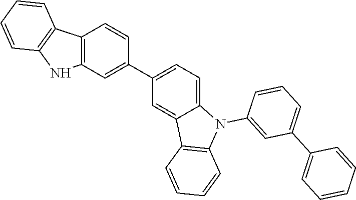 [SMILES: c1ccc(-c2cccc(-n3c4ccccc4c4cc(-c5ccc6c(c5)[nH]c5ccccc56)ccc43)c2)cc1] | | 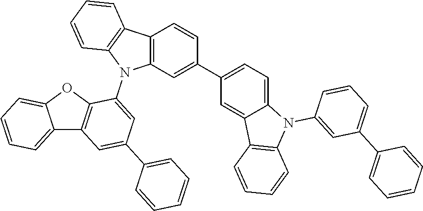 [SMILES: c1ccc(-c2cccc(-n3c4ccccc4c4cc(-c5ccc6c7ccccc7n(-c7cc(-c8ccccc8)cc8c7oc7ccccc78)c6c5)ccc43)c2)cc1] | 76% |
| 1-155 | 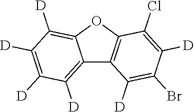 [SMILES: [2H]c1c([2H])c([2H])c2c(oc3c(Cl)c([2H])c(Br)c([2H])c32)c1[2H]] | 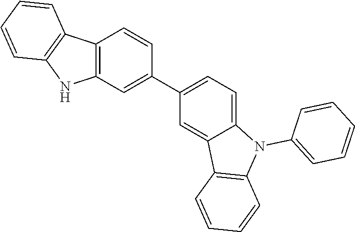 [SMILES: c1ccc(-n2c3ccccc3c3cc(-c4ccc5c(c4)[nH]c4ccccc45)ccc32)cc1] | 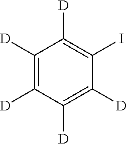 [SMILES: [2H]c1c([2H])c([2H])c(I)c([2H])c1[2H]] | 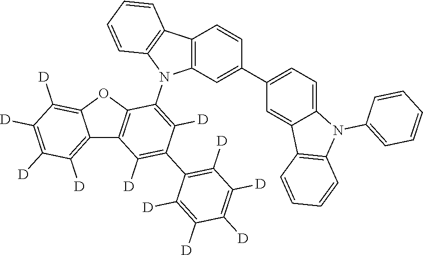 [SMILES: [2H]c1c([2H])c([2H])c(-c2c([2H])c(-n3c4ccccc4c4ccc(-c5ccc6c(c5)c5ccccc5n6-c5ccccc5)cc43)c3oc4c([2H])c([2H])c([2H])c([2H])c4c3c2[2H])c([2H])c1[2H]] | 80% |
| 1-156 | | 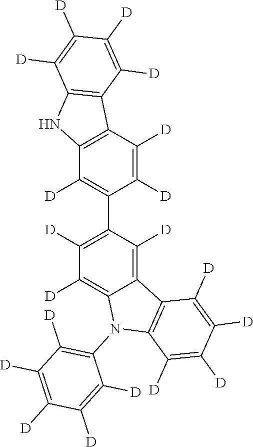 [SMILES: [2H]c1c([2H])c([2H])c(-n2c3c([2H])c([2H])c([2H])c([2H])c3c3c([2H])c(-c4c([2H])c([2H])c5c([nH]c6c([2H])c([2H])c([2H])c([2H])c65)c4[2H])c([2H])c([2H])c32)c([2H])c1[2H]] | | 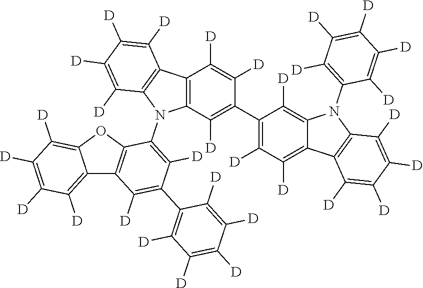 [SMILES: [2H]c1c([2H])c([2H])c(-c2c([2H])c(-n3c4c([2H])c([2H])c([2H])c([2H])c4c4c([2H])c([2H])c(-c5c([2H])c([2H])c6c7c([2H])c([2H])c([2H])c([2H])c7n(-c7c([2H])c([2H])c([2H])c([2H])c7[2H])c6c5[2H])c([2H])c43)c3oc4c([2H])c([2H])c([2H])c([2H])c4c3c2[2H])c([2H])c1[2H]] | 78% |
| 1-159 | 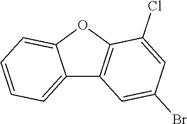 [SMILES: Clc1cc(Br)cc2c1oc1ccccc12] | 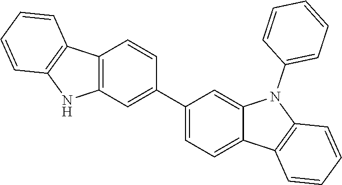 [SMILES: c1ccc(-n2c3ccccc3c3ccc(-c4ccc5c(c4)[nH]c4ccccc45)cc32)cc1] | 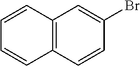 [SMILES: Brc1ccc2ccccc2c1] | 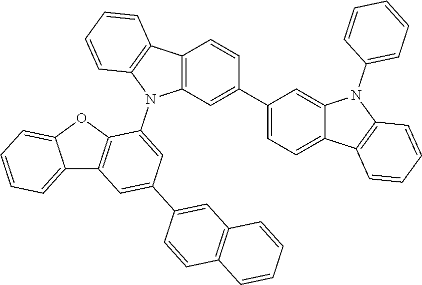 [SMILES: c1ccc(-n2c3ccccc3c3ccc(-c4ccc5c6ccccc6n(-c6cc(-c7ccc8ccccc8c7)cc7c6oc6ccccc67)c5c4)cc32)cc1] | 86% |
| 1-163 | | 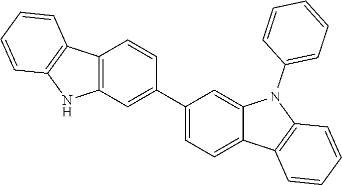 [SMILES: c1ccc(-n2c3ccccc3c3ccc(-c4ccc5c(c4)[nH]c4ccccc45)cc32)cc1] | 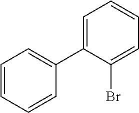 [SMILES: Brc1ccccc1-c1ccccc1] | 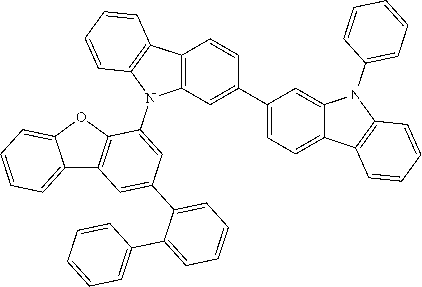 [SMILES: c1ccc(-c2ccccc2-c2cc(-n3c4ccccc4c4ccc(-c5ccc6c7ccccc7n(-c7ccccc7)c6c5)cc43)c3oc4ccccc4c3c2)cc1] | 85% |
| 1-167 | | 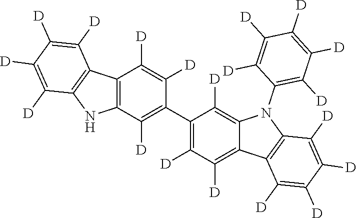 [SMILES: [2H]c1c([2H])c([2H])c(-n2c3c([2H])c([2H])c([2H])c([2H])c3c3c([2H])c([2H])c(-c4c([2H])c([2H])c5c([nH]c6c([2H])c([2H])c([2H])c([2H])c65)c4[2H])c([2H])c32)c([2H])c1[2H]] | 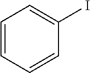 | 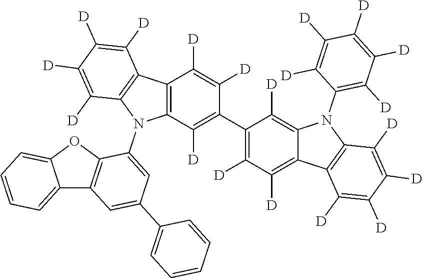 [SMILES: [2H]c1c([2H])c([2H])c(-n2c3c([2H])c([2H])c([2H])c([2H])c3c3c([2H])c([2H])c(-c4c([2H])c([2H])c5c6c([2H])c([2H])c([2H])c([2H])c6n(-c6cc(-c7ccccc7)cc7c6oc6ccccc67)c5c4[2H])c([2H])c32)c([2H])c1[2H]] | 70% |
| 1-170 | 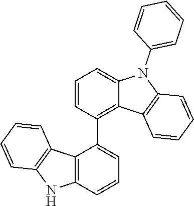 [SMILES: c1ccc(-n2c3ccccc3c3c(-c4cccc5[nH]c6ccccc6c45)cccc32)cc1] | | | 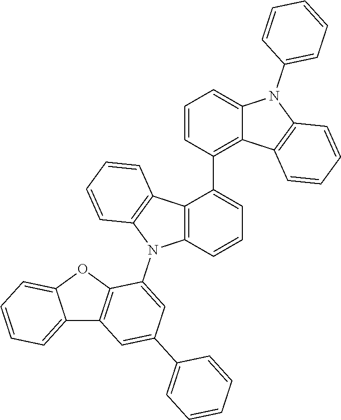 [SMILES: c1ccc(-c2cc(-n3c4ccccc4c4c(-c5cccc6c5c5ccccc5n6-c5ccccc5)cccc43)c3oc4ccccc4c3c2)cc1] | 84% |
| 1-171 | | | 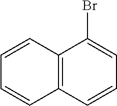 [SMILES: Brc1cccc2ccccc12] | 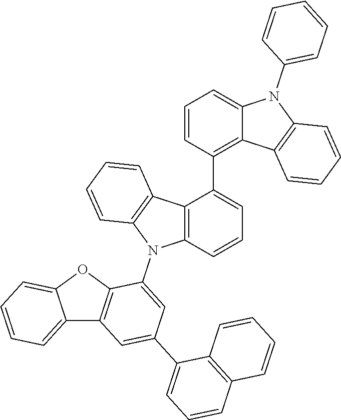 [SMILES: c1ccc(-n2c3ccccc3c3c(-c4cccc5c4c4ccccc4n5-c4cc(-c5cccc6ccccc56)cc5c4oc4ccccc45)cccc32)cc1] | 86% |
| 1-173 | | | 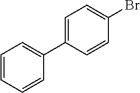 [SMILES: Brc1ccc(-c2ccccc2)cc1] | 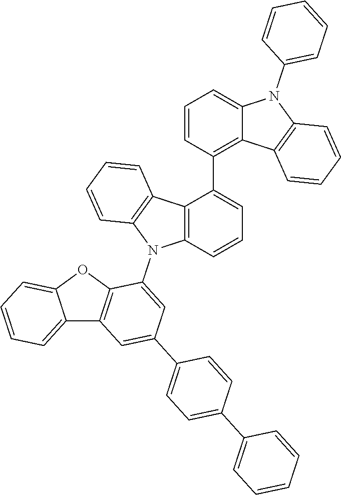 [SMILES: c1ccc(-c2ccc(-c3cc(-n4c5ccccc5c5c(-c6cccc7c6c6ccccc6n7-c6ccccc6)cccc54)c4oc5ccccc5c4c3)cc2)cc1] | 90% |
| 1-174 | | | 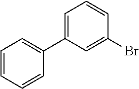 [SMILES: Brc1cccc(-c2ccccc2)c1] | 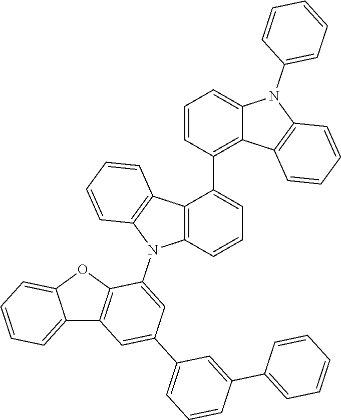 [SMILES: c1ccc(-c2cccc(-c3cc(-n4c5ccccc5c5c(-c6cccc7c6c6ccccc6n7-c6ccccc6)cccc54)c4oc5ccccc5c4c3)c2)cc1] | 78% |
| 1-179 | | 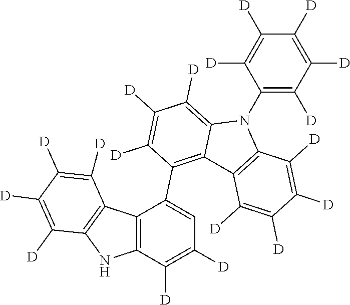 [SMILES: [2H]c1cc(-c2c([2H])c([2H])c([2H])c3c2c2c([2H])c([2H])c([2H])c([2H])c2n3-c2c([2H])c([2H])c([2H])c([2H])c2[2H])c2c([nH]c3c([2H])c([2H])c([2H])c([2H])c32)c1[2H]] | 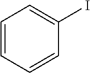 | 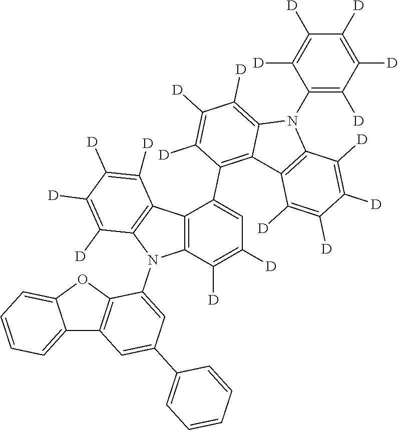 [SMILES: [2H]c1cc(-c2c([2H])c([2H])c([2H])c3c2c2c([2H])c([2H])c([2H])c([2H])c2n3-c2c([2H])c([2H])c([2H])c([2H])c2[2H])c2c3c([2H])c([2H])c([2H])c([2H])c3n(-c3cc(-c4ccccc4)cc4c3oc3ccccc34)c2c1[2H]] | 79% |

A compound other than the compound shown in Table 1 among Compounds 1-1 to 1-180 was also prepared in the same manner as in the method described in the above-described Preparation Examples.

### <Preparation Example 2> Preparation of Compound 2-1

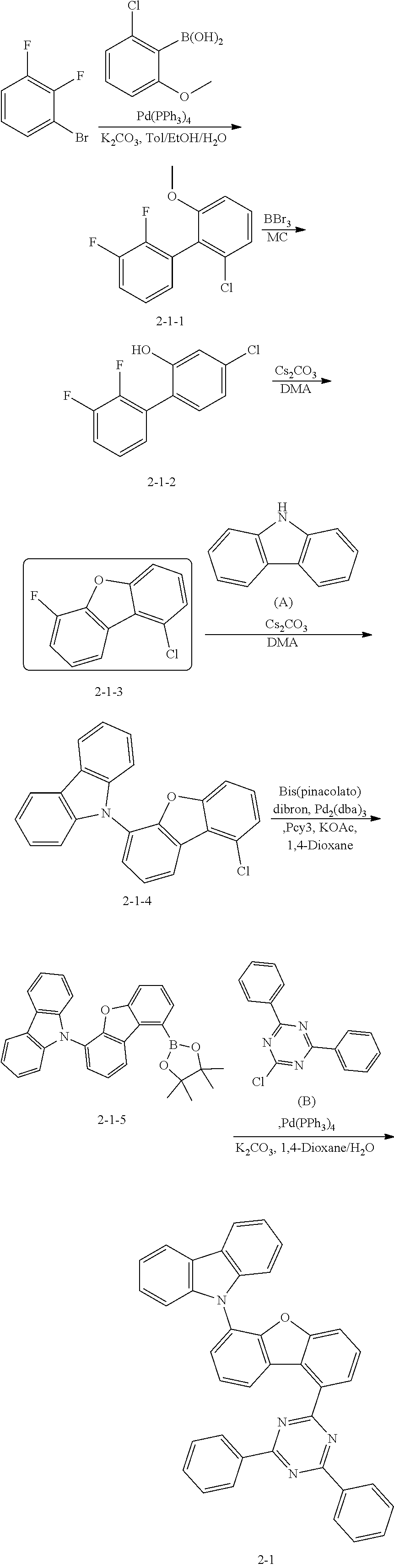

### Preparation of Compound 2-1-1

A mixture of 1-bromo-2,3-difluorobenzene (40.5 g, 209 mmol), (2-chloro-6-methoxyphenyl) boronic acid (43 g, 230 mmol), tetrakis(triphenylphosphine) palladium (0) (24 g, 20.9 mmol), potassium carbonate (57.9 g, 419 mmol), and toluene/ethanol/water (500 ml/100 ml/100 ml) was refluxed at 110° C. in a one-neck round-bottom flask. The resulting product was extracted with dichloromethane and dried over MgSO₄. The product was filtered by silica gel, and then concentrated to obtain Compound 2-1-1. (40.8 g, 76%)

### Preparation of Compound 2-1-2

A mixture of 2′-chloro-2,3-difluoro-6′-methoxy-1,1′-biphenyl (40.8 g, 160 mmol) and MC (600 ml) was cooled down to a temperature of 0° C. in a one-neck round-bottom flask, BBr₃ (30 mL, 320 mmol) was added dropwise thereto, and the resulting mixture was warmed to room temperature and stirred for 1 hour. The reaction was terminated with distilled water, and the resulting product was extracted with dichloromethane and dried over MgSO₄. Column purification MC:HX=1:1 was performed to obtain Compound 2-1-2. (21 g, 54%)

### Preparation of Compound 2-1-3

A mixture of dimethylacetamide (200 ml) with 4-chloro-2′,3′-difluoro-[1,1′-biphenyl]-2-ol (21 g, 87.2 mmol) and CS₂CO₃ (71 g, 218 mmol) was stirred at 120° C. in a one-neck round-bottom flask. After the mixture was cooled, the mixture was filtered, the solvent of the filtrate was removed, and then column purification HX:MC=4:1 was performed to obtain Compound 2-1-3. (17 g, 88%)

### Preparation of Compound 2-1-4

A mixture of dimethylacetamide (60 ml) with 1-chloro-6-fluorodibenzo-[b, d]furan (6 g, 27.19 mmol), 9H-carbazole (5 g, 29.9 mmol) and Cs₂CO₃ (22 g, 101.7 mmol) was refluxed at 170° C. for 12 hours in a one-neck round-bottom flask. After the mixture was cooled, the mixture was filtered, the solvent of the filtrate was removed, and then column purification HX:MC=3:1 was performed to obtain Compound 2-1-4. (9 g, 90%)

### Preparation of Compound 2-1-5

A mixture of 1,4-dioxane (100 ml) with 9-(9-chlorodibenzo[b, d]furan-4-yl)-9H-carbazole (9 g, 24.4 mmol), bis(pinacolato)diboron (12.4 g, 48.9 mmol), Pcy3 (1.37 g, 4.89 mmol), potassium acetate (7.1 g, 73 mmol), and Pd₂(dba)₃ (2.2 g, 2.44 mmol) was refluxed at 140° C. in a one-neck round-bottom flask. After the mixture was cooled, the filtered filtrate was concentrated, and column purification HX:MC=3:1 was performed to obtain Compound 2-1-5. (7.2 g, 64%)

### Preparation of Compound 2-1

A mixture of 9-(9-(4,4,5,5-tetramethyl-1,3,2-dioxaborolan-2-yl)dibenzo[b, d]furan-4-yl)-9H-carbazole (7.2 g, 15.6 mmol), 2-chloro-4,6-diphenyl-1,3,5-triazine (5 g, 18.8 mmol), tetrakis(triphenylphosphine) palladium (0) (1.8 g, 1.56 mmol), potassium carbonate (4.3 g, 31.2 mmol), and 1,4-dioxane/water (100 ml/25 ml) was refluxed at 120° C. for 4 hours in a one-neck round-bottom flask. Thereafter, the mixture was filtered at 120° C., and then washed with 1,4-dioxane, distilled water, and MeOH to obtain Compound 2-1 (C). (6.6 g, 75%)

The following Compound C was synthesized in the same manner as in the preparation of Compound 2-1, except that A and B in the following Tables 2 to 8 were used as intermediates in Preparation Example 2.

TABLE 2

| Com- | | | | |
|---|---|---|---|---|
| pound | A | B | C | Yield |
| 2-2 | 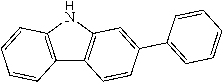 | 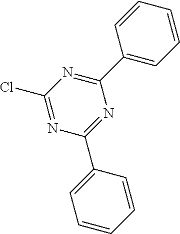 | 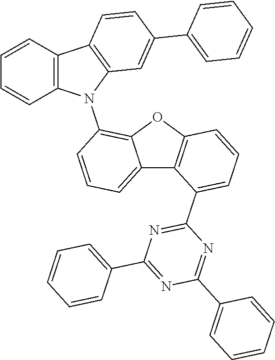 | 69% |
| 2-3 | 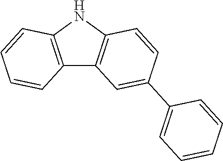 | 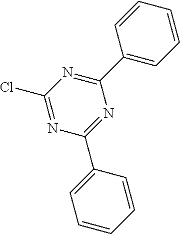 | 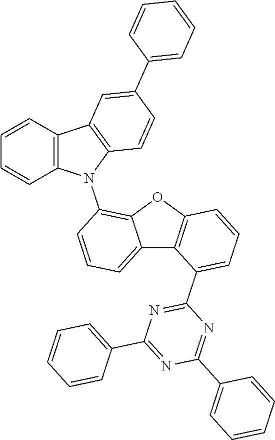 | 72% |
| 2-5 | 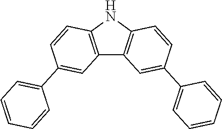 | 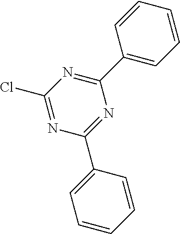 | 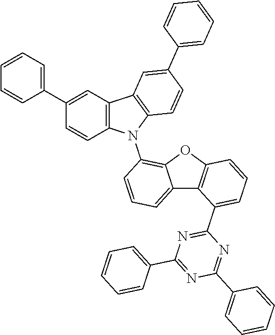 | 70% |
| 2-7 | 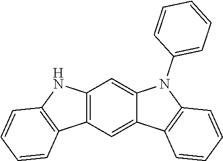 | 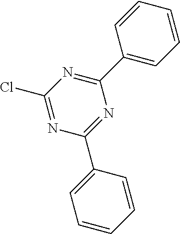 | 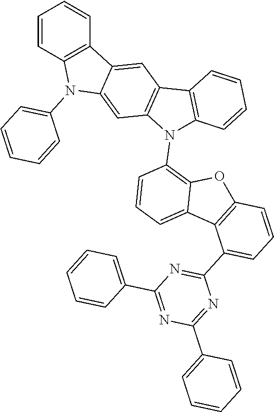 | 61% |
| 2-10 | 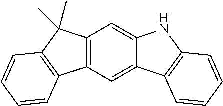 | 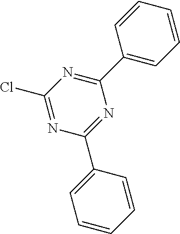 | 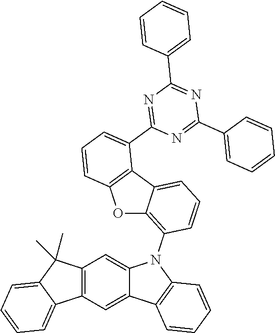 | 63% |
| 2-17 | 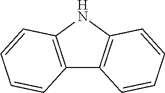 | 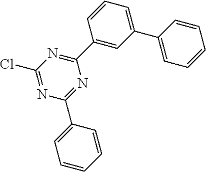 | 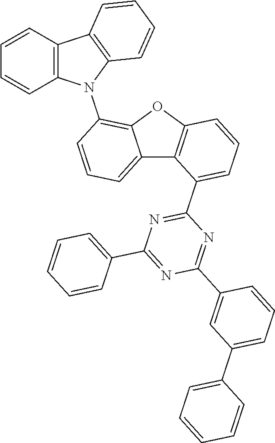 | 64% |

TABLE 3

| Compound | A | B | C | Yield |
|---|---|---|---|---|
| 2-18 | 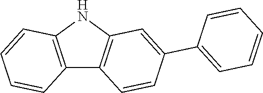 | 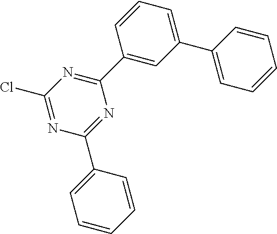 | 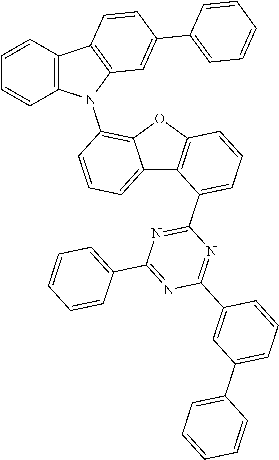 | 66% |
| 2-19 | 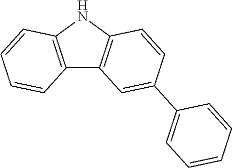 | 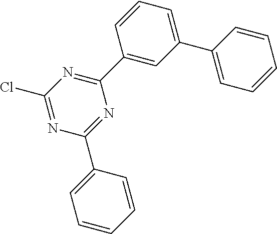 | 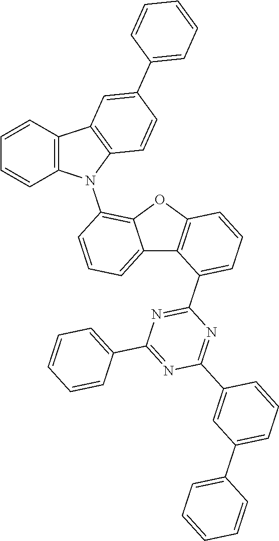 | 69% |
| 2-22 | 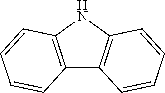 | 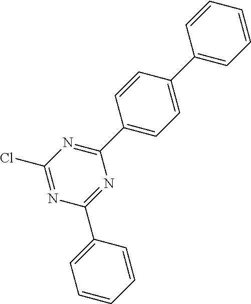 | 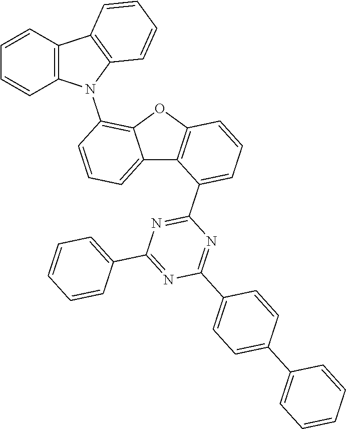 | 66% |
| 2-28 | 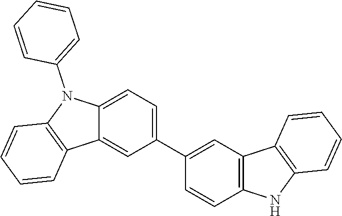 | 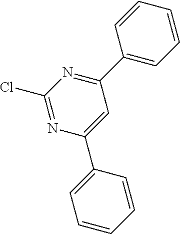 | 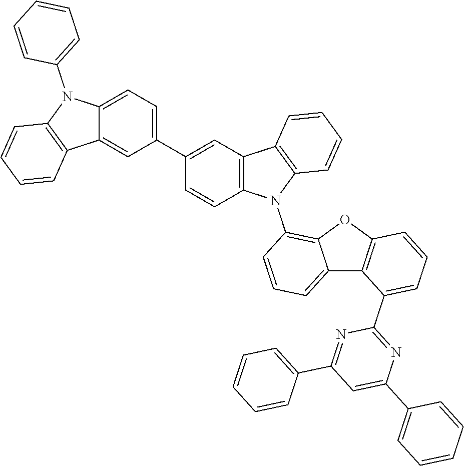 | 69% |
| 2-29 | 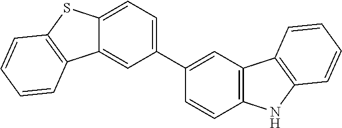 | 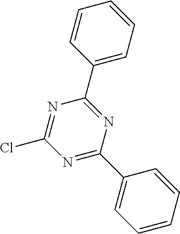 | 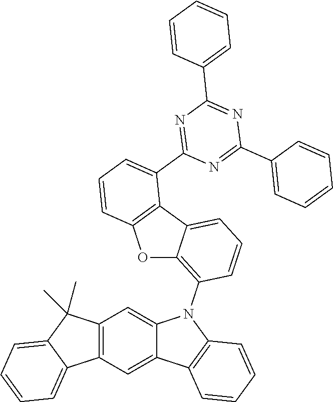 | 58% |
| 2-34 | 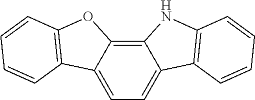 | 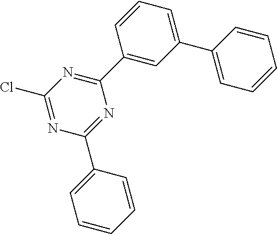 | 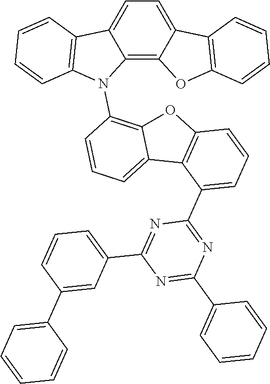 | 62% |

TABLE 4

| Compound | A | B | C | Yield |
|---|---|---|---|---|
| 2-38 | 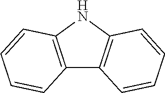 | 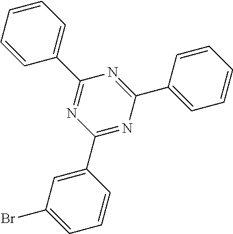 | 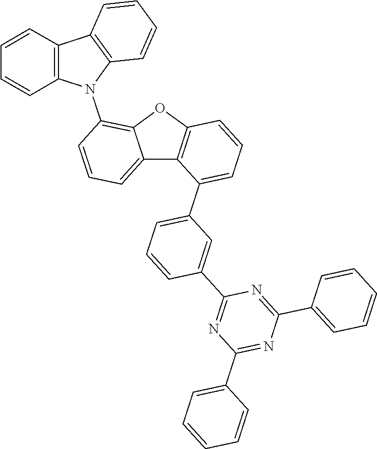 | 70% |
| 2-39 | 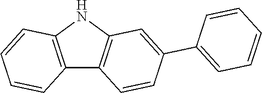 | 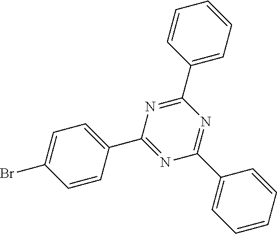 | 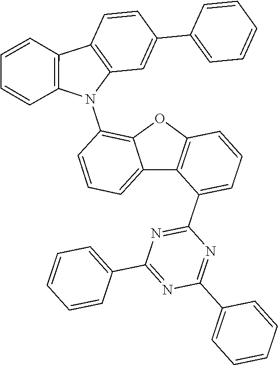 | 71% |
| 2-43 | 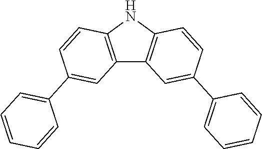 | 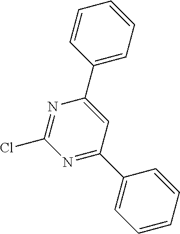 | 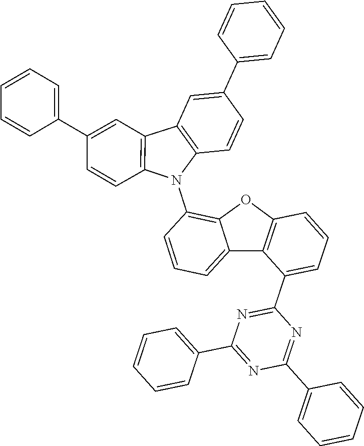 | 68% |
| 2-45 | 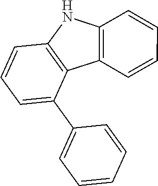 | 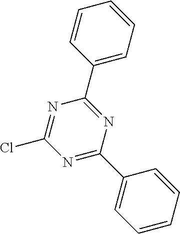 | 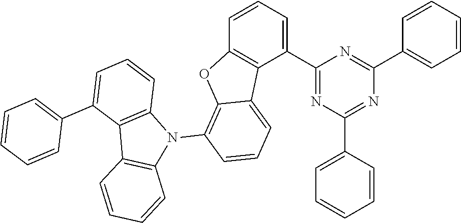 | 42% |
| 2-48 | 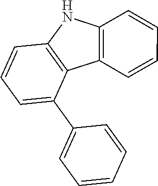 | 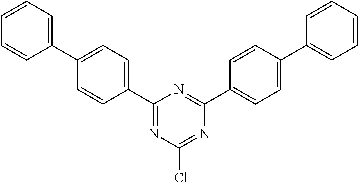 | 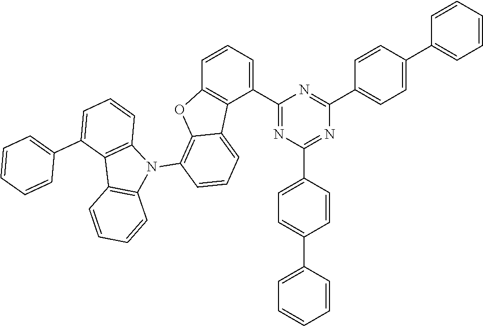 | 48% |
| 2-51 | 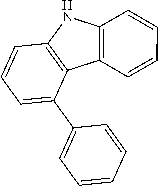 | 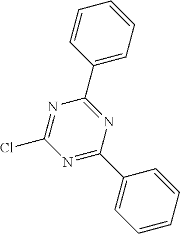 | 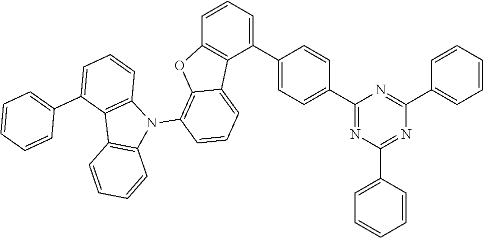 | 53% |

TABLE 5

| Compound | A | B | C | Yield |
|---|---|---|---|---|
| 2-53 | 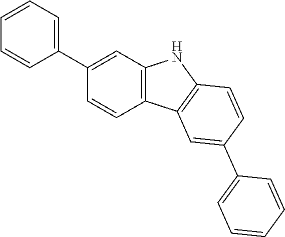 | 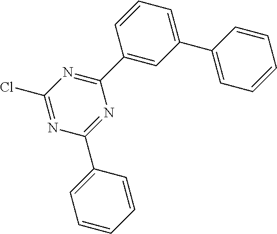 | 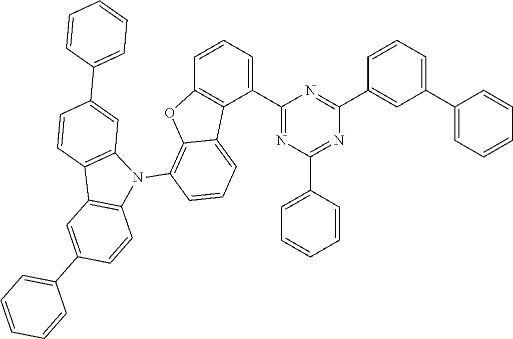 | 51 % |
| 2-59 | 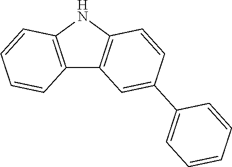 | 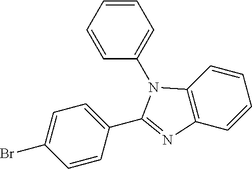 | 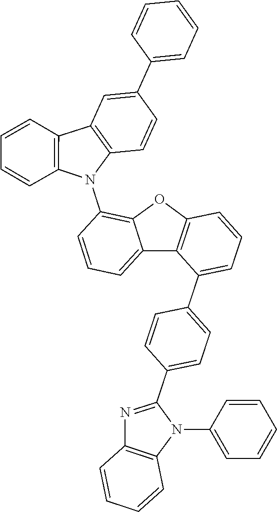 | 66% |
| 2-62 | 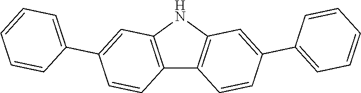 | 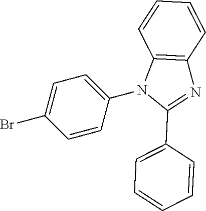 | 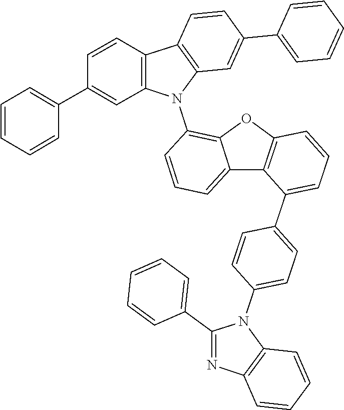 | 65% |
| 2-65 | 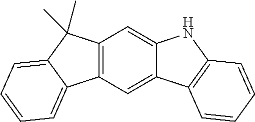 | 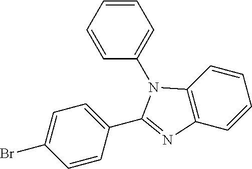 | 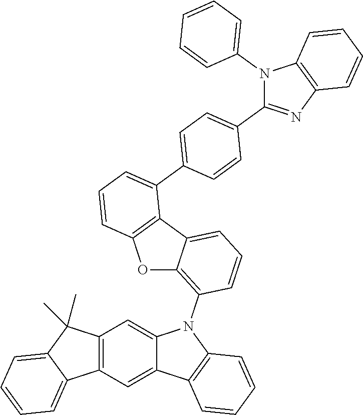 | 67% |
| 2-67 | 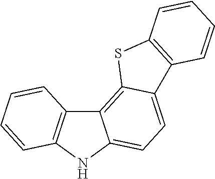 | 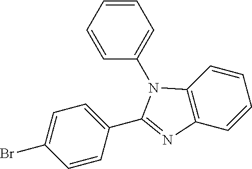 | 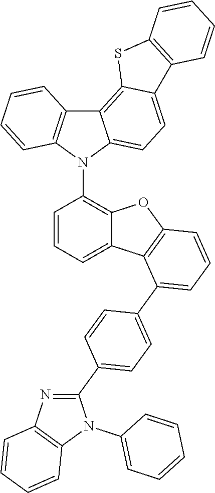 | 61% |
| 2-70 | 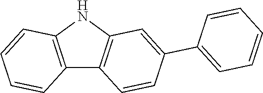 | 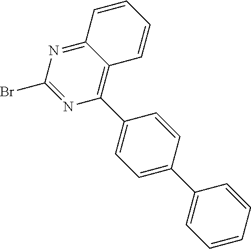 | 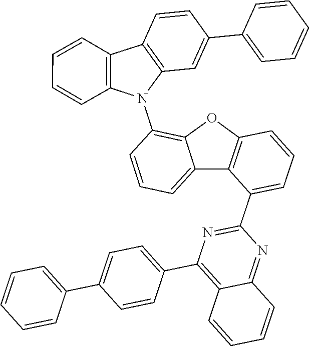 | 69% |

TABLE 6

| Compound | A | B |
|---|---|---|
| 2-71 | 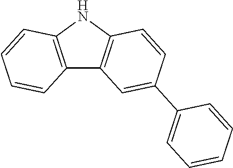 | 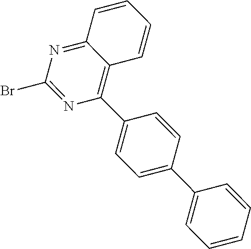 |
| 2-75 | 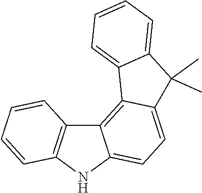 | 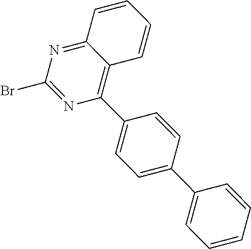 |
| 2-77 | 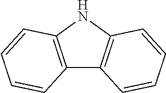 | 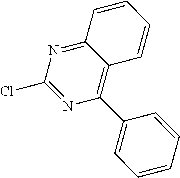 |
| 2-80 | 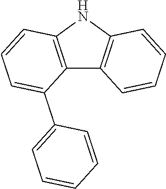 | 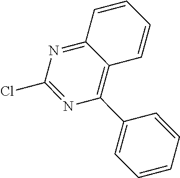 |
| 2-83 | 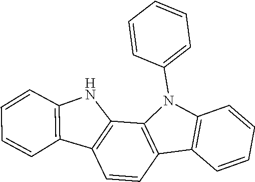 | 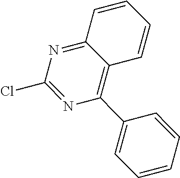 |
| 2-86 | 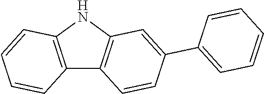 | 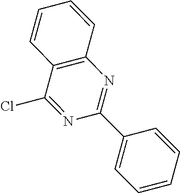 |

| Compound | C | Yield |
|---|---|---|
| 2-71 | 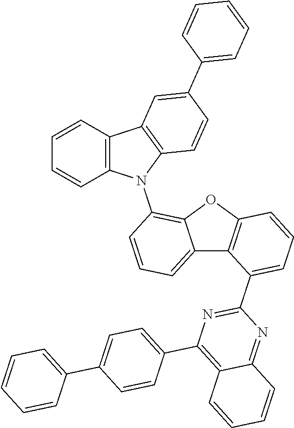 | 63% |
| 2-75 | 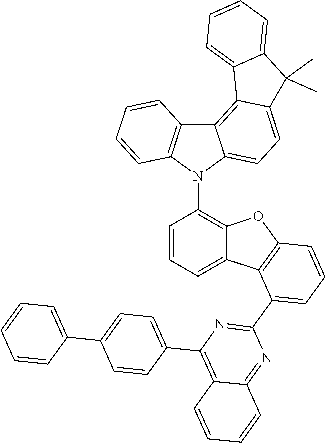 | 64% |
| 2-77 | 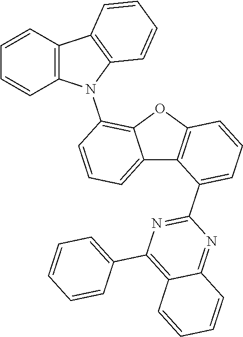 | 64% |
| 2-80 | 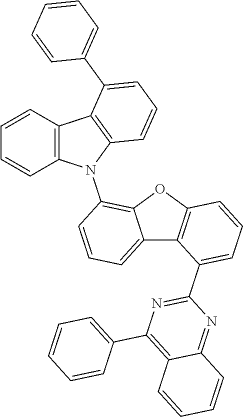 | 70% |
| 2-83 | 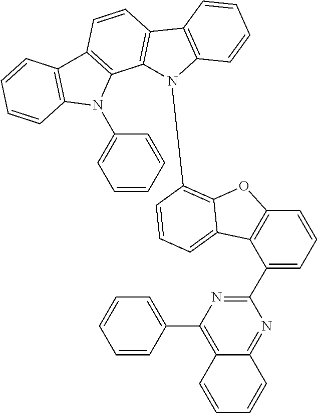 | 68% |
| 2-86 | 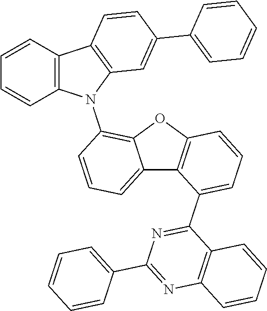 | 60% |

TABLE 7

| Compound | A | B |
|---|---|---|
| 2-90 | 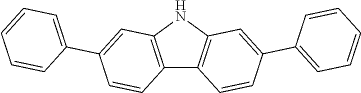 | 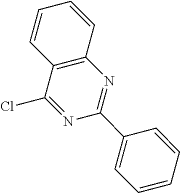 |
| 2-92 | 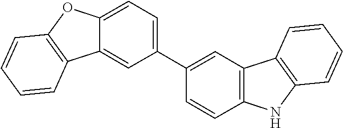 | 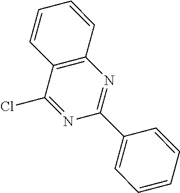 |
| 2-95 | 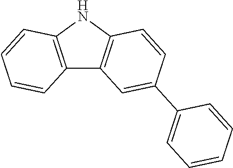 | 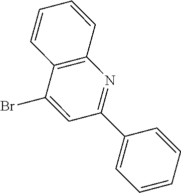 |
| 2-100 | 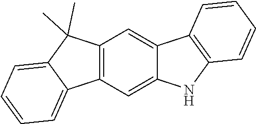 | 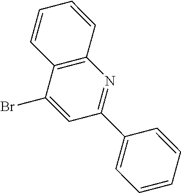 |
| 2-101 | 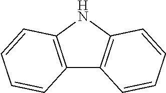 | 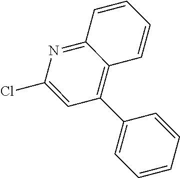 |
| 2-110 | 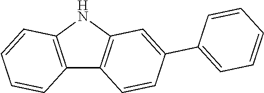 | 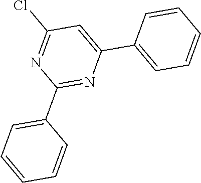 |

| Compound | C | Yield |
|---|---|---|
| 2-90 | 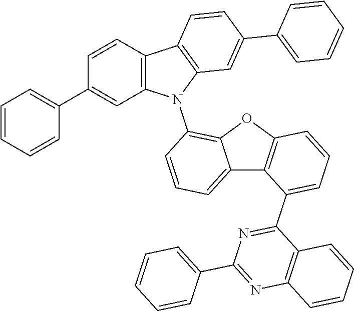 | 66% |
| 2-92 | 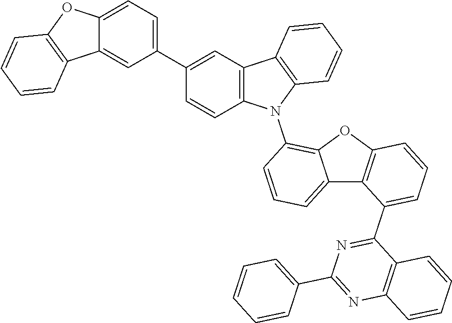 | 61% |
| 2-95 | 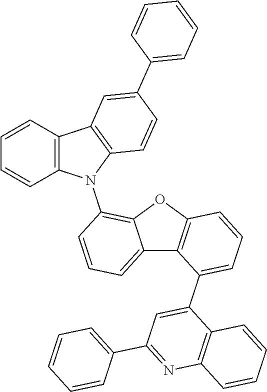 | 51% |
| 2-100 | 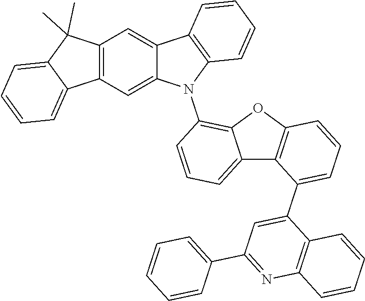 | 54% |
| 2-101 | 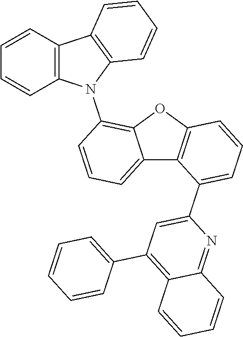 | 49% |
| 2-110 | 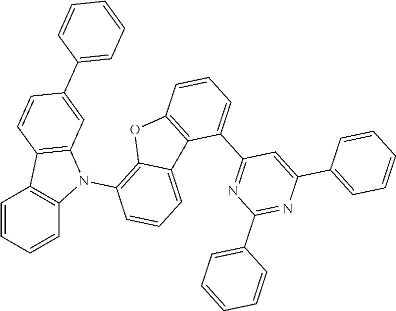 | 54% |

TABLE 8

| Compound | A | B |
|---|---|---|
| 2-113 | 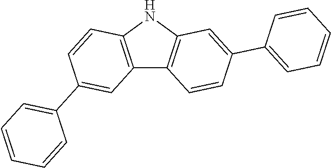 | 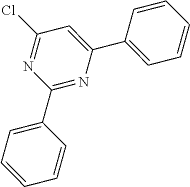 |
| 2-118 | 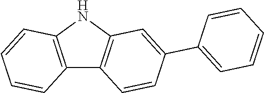 | 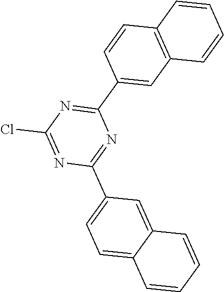 |
| 2-119 | 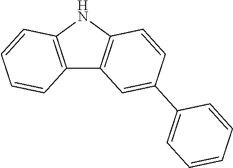 | 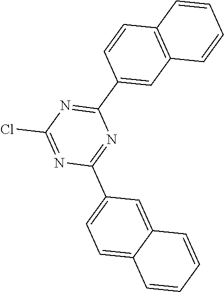 |
| 2-121 | 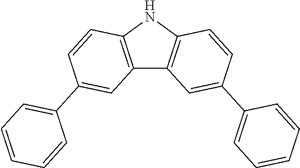 | 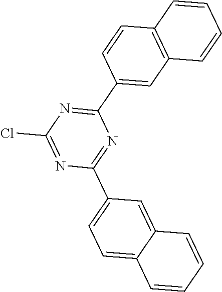 |
| 2-125 | 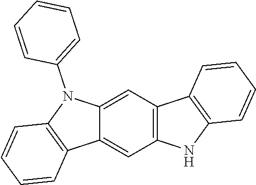 | 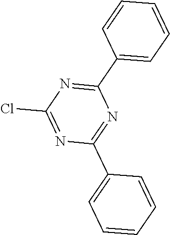 |
| 2-127 | 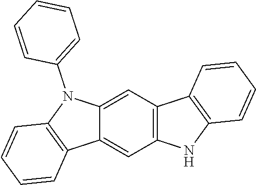 | 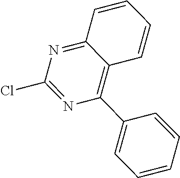 |
| 2-257 | 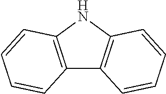 | 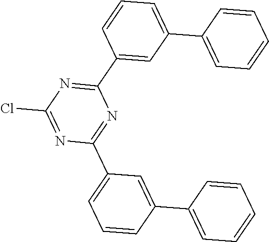 |
| 2-258 | 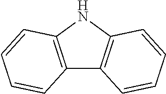 | 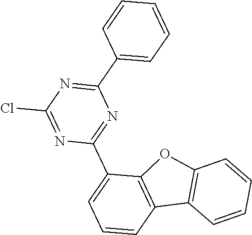 |
| 2-261 | | |
| 2-286 | | |

| Compound | C | Yield |
|---|---|---|
| 2-113 | 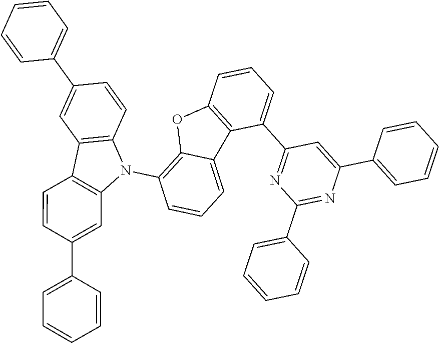 | 69% |
| 2-118 | 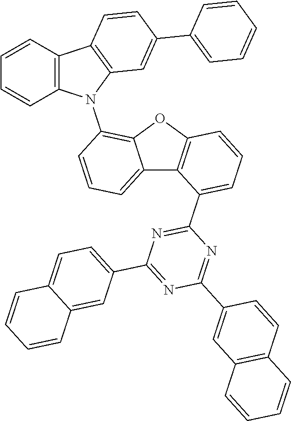 | 60% |
| 2-119 | 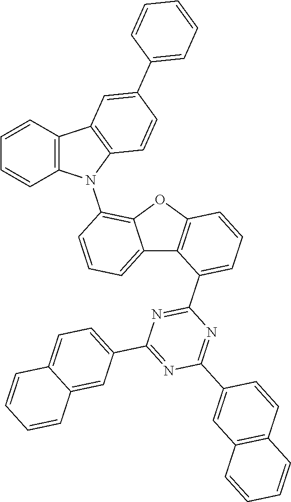 | 62% |
| 2-121 | 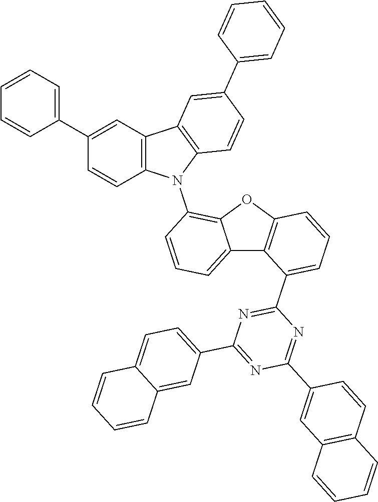 | 63% |
| 2-125 | 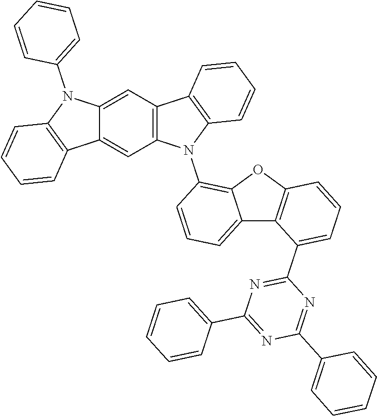 | 70% |
| 2-127 | 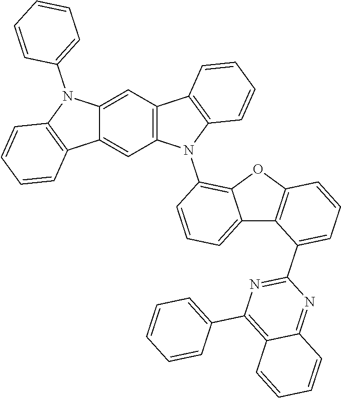 | 64% |
| 2-257 | 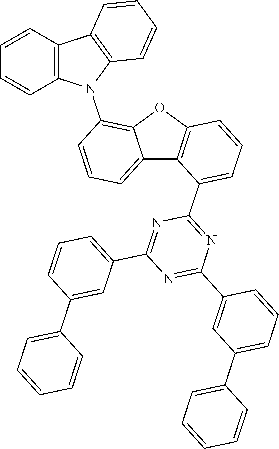 | 63% |
| 2-258 | 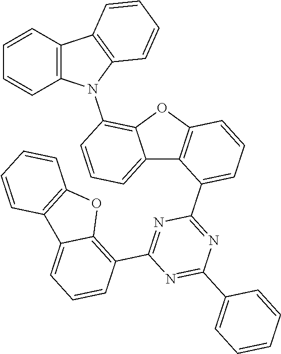 | 69% |
| 2-261 | | 68% |
| 2-286 | | 71% |

### <Preparation Example 3> Preparation of Compound 2-129

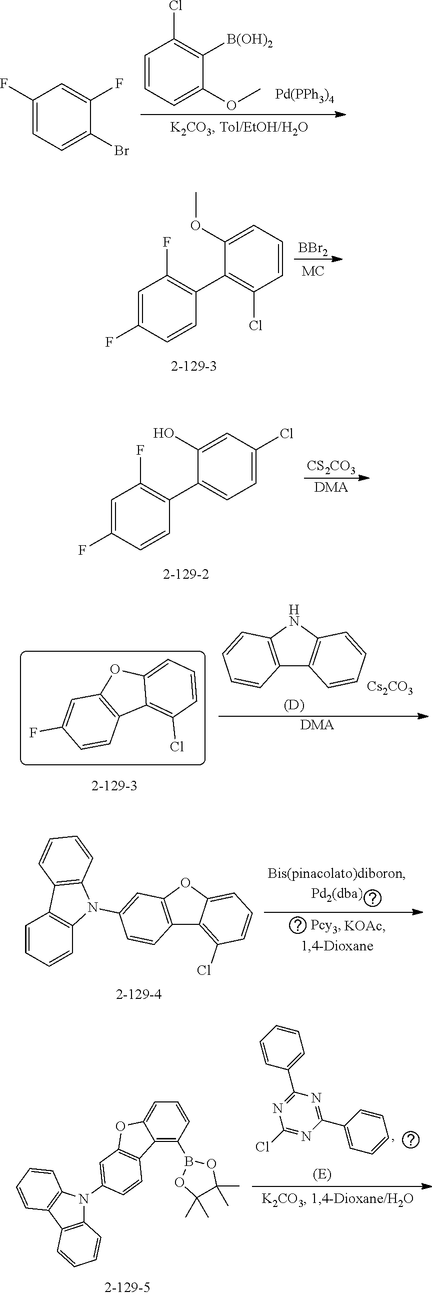

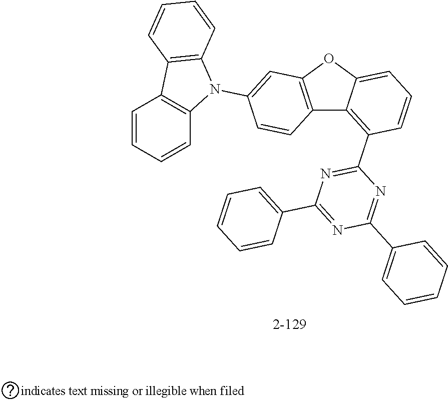 [SMILES: c1ccc(-c2nc(-c3ccccc3)nc(-c3cccc4oc5cc(-n6c7ccccc7c7ccccc76)ccc5c34)n2)cc1]

### Preparation of Compound 2-129-1

A mixture of 1-bromo-2,4-difluorobenzene (40 g, 207 mmol), (2-chloro-6-methoxyphenyl) boronic acid (42.4 g, 227 mmol), tetrakis(triphenylphosphine) palladium (0) (23 g, 20.7 mmol), potassium carbonate (57 g, 414 mmol), and toluene/ethanol/water (600 ml/150 ml/150 ml) was refluxed at 110° C. in a one-neck round-bottom flask.

The resulting product was extracted with dichloromethane and dried over MgSO₄. The product was filtered by silica gel, and then concentrated to obtain Compound 2-129-1. (50 g, 94%)

### Preparation of Compound 2-129-2

A mixture of 2′-chloro-2,4-difluoro-6′-methoxy-1,1′-biphenyl (50 g, 196 mmol) and dichloromethane (700 ml) was cooled down to a temperature of 0° C. in a one-neck round-bottom flask, BBr₃ (28.3 mL, 294 mmol) was added dropwise thereto, and the resulting mixture was warmed to room temperature and stirred for 2 hours.

The reaction was terminated with distilled water, and the resulting product was extracted with dichloromethane and dried over MgSO₄. The product was filtered by silica gel to obtain Compound 2-129-2. (27.5 g, 58%)

### Preparation of Compound 2-129-3

A mixture of dimethylacetamide (300 ml) with 4-chloro-2′,4′-difluoro-[1,1′-biphenyl]-2-ol (27 g, 114 mmol) and CS₂CO₃ (83 g, 285 mmol) was stirred at 120° C. in a one-neck round-bottom flask. After the mixture was cooled, the mixture was filtered, the solvent of the filtrate was removed, and then the residue was filtered with silica gel to obtain Compound 2-129-3. (23 g, 92%)

### Preparation of Compound 2-129-4

A mixture of dimethylacetamide (60 ml) with 1-chloro-7-fluorodibenzo-[b, d]furan (5.5 g, 24.9 mmol), 9H-carbazole (4.58 g, 27.4 mmol) and CS₂CO₃ (20 g, 62 mmol) was refluxed at 170° C. for 6 hours in a one-neck round-bottom flask. After the mixture was cooled, the mixture was filtered, the solvent of the filtrate was removed, and then column purification HX:MC=3:1 was performed to obtain Compound 2-129-4. (7.6 g, 83%)

### Preparation of Compound 2-129-5

A mixture of 1,4-dioxane (80 ml) with 9-(9-chlorodibenzo[b, d]furan-3-yl)-9H-carbazole (7.5 g, 20.3 mmol), bis(pinacolato)diboron (10.3 g, 40.7 mmol), Pcy3 (1.14 g, 4.07 mmol), potassium acetate (5.97 g, 60.9 mmol), and Pd₂(dba)₃ (1.85 g, 2.03 mmol) was refluxed at 140° C. in a one-neck round-bottom flask. After the mixture was cooled, the filtered filtrate was concentrated, and column purification HX:MC=2:1 was performed to obtain Compound 2-129-5 (6.5 g, 70%).

### Preparation of Compound 2-129

A mixture of 9-(9-(4,4,5,5-tetramethyl-1,3,2-dioxaborolan-2-yl)dibenzo[b, d]furan-3-yl)-9H-carbazole (6.5 g, 14.1 mmol), 2-chloro-4,6-diphenyl-1,3,5-triazine (4.54 g, 16.9 mmol), tetrakis(triphenylphosphine) palladium (0) (1.6 g, 1.41 mmol), potassium carbonate (3.9 g, 28.2 mmol), and 1,4-dioxane/water (80 ml/28.2 ml) was refluxed at 120° C. for 4 hours in a one-neck round-bottom flask. Thereafter, the mixture was filtered at 120° C., and then washed with 1,4-dioxane, distilled water, and methanol (MeOH) to obtain Compound 2-129 (F) (5.4 g, 68).

The following Compound F was synthesized in the same manner as in the preparation of Compound 2-129, except that D and E in the following Tables 9 to 15 were used as intermediates in Preparation Example 3.

TABLE 9

| Compound | D | E |
|---|---|---|
| 2-130 | 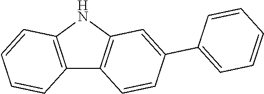 | 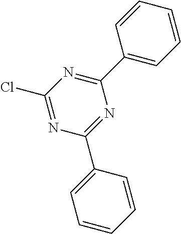 |
| 2-131 | 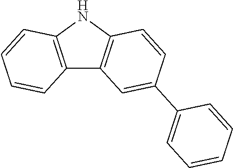 | 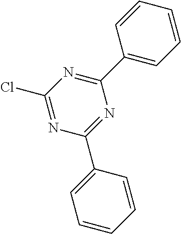 |
| 2-133 | 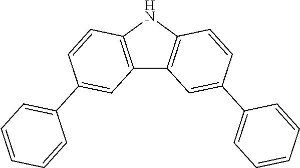 | 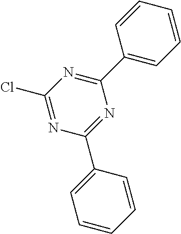 |
| 2-135 | 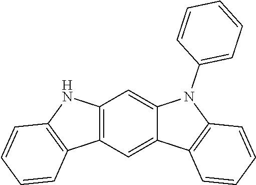 | 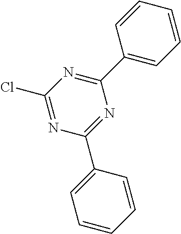 |
| 2-138 | 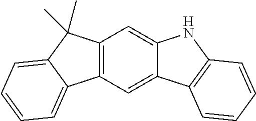 | 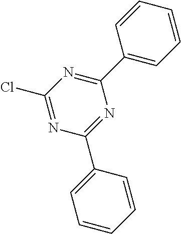 |
| 2-145 | 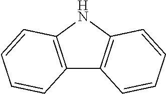 | 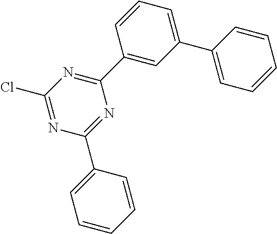 |

| Compound | F | Yield |
|---|---|---|
| 2-130 | 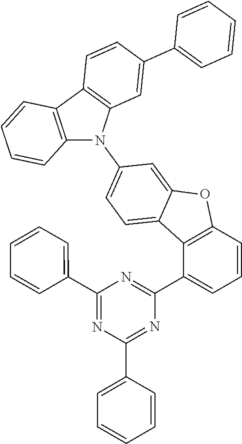 | 69% |
| 2-131 | 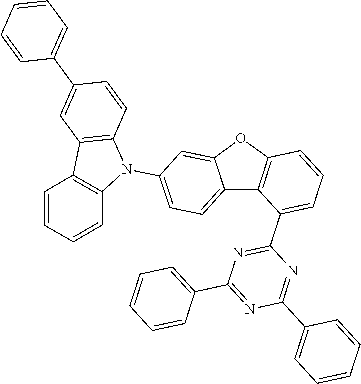 | 72% |
| 2-133 | 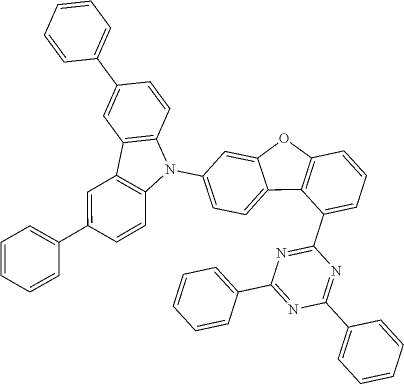 | 70% |
| 2-135 | 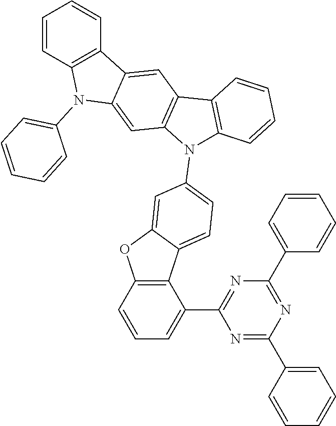 | 61% |
| 2-138 | 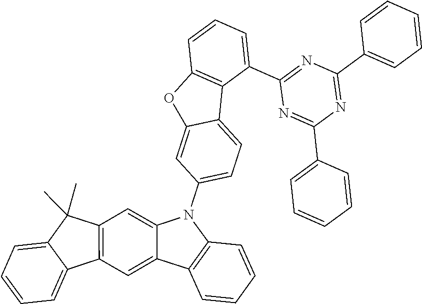 | 63% |
| 2-145 | 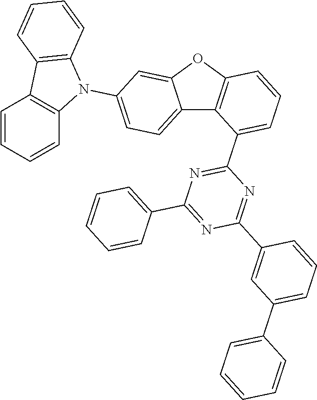 | 64% |

TABLE 10

| Compound | D | E |
|---|---|---|
| 2-146 | 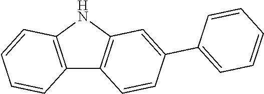 | 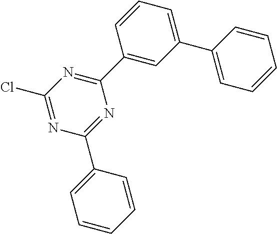 |
| 2-147 | 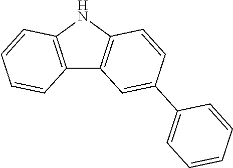 | 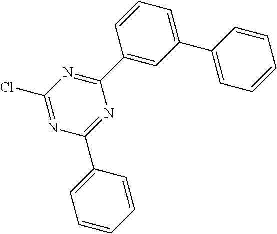 |
| 2-150 | 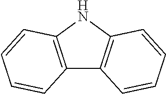 | 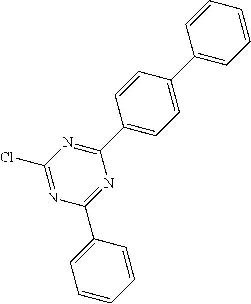 |
| 2-156 | 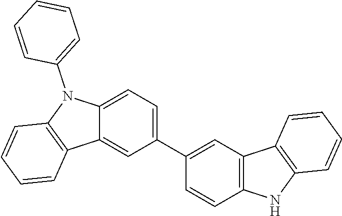 | 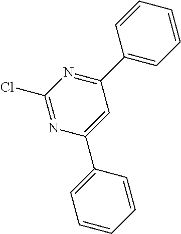 |
| 2-157 | 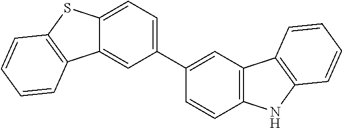 | 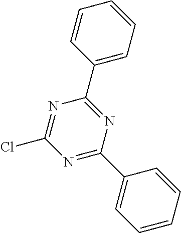 |
| 2-162 | 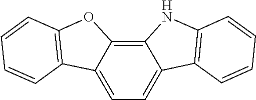 | 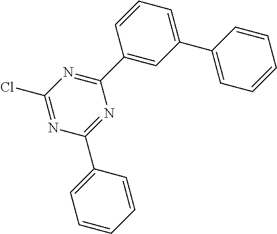 |

| Compound | F | Yield |
|---|---|---|
| 2-146 | 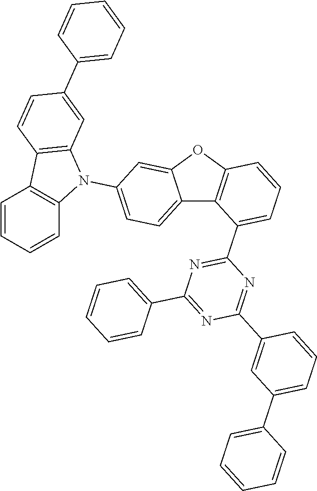 | 70% |
| 2-147 | 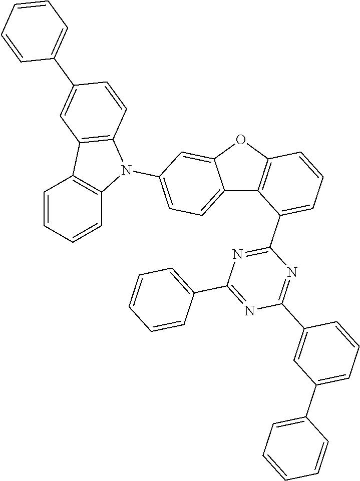 | 68% |
| 2-150 | 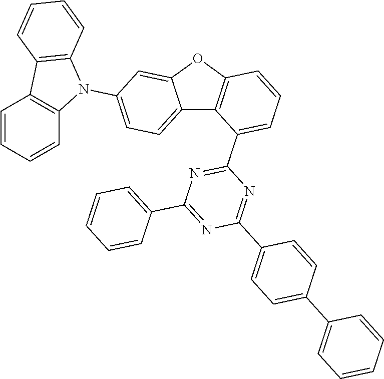 | 66% |
| 2-156 | 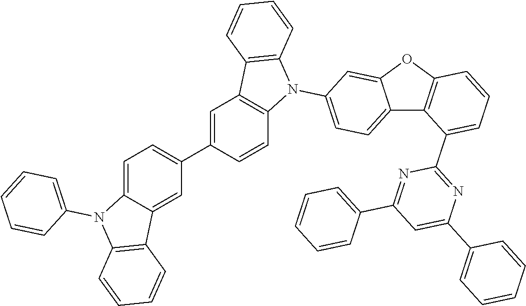 | 69% |
| 2-157 | 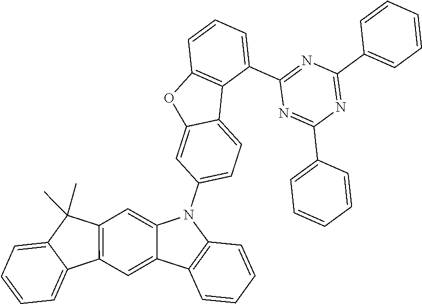 | 58% |
| 2-162 | 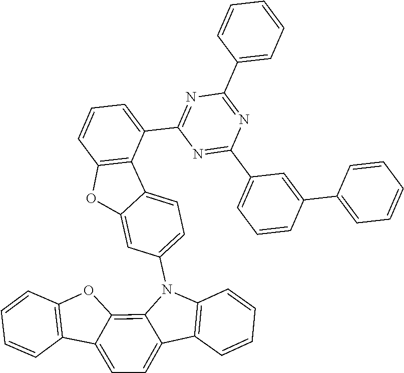 | 62% |

TABLE 11

| Compound | D | E |
|---|---|---|
| 2-166 | 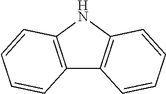 | 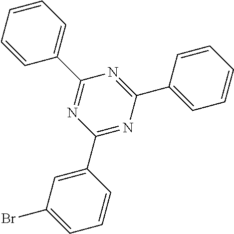 |
| 2-167 | 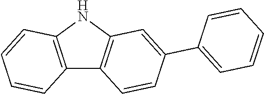 | 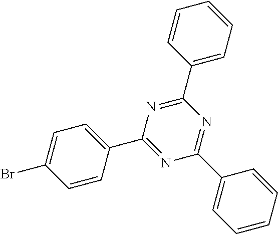 |
| 2-171 | 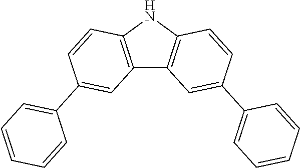 | 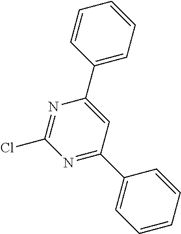 |
| 2-173 | 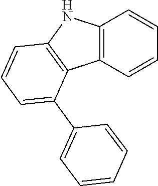 | 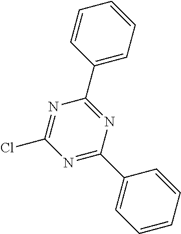 |
| 2-176 | 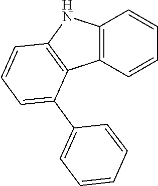 | 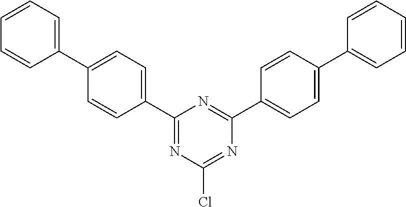 |
| 2-179 | 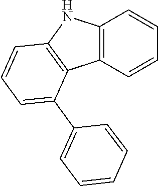 | 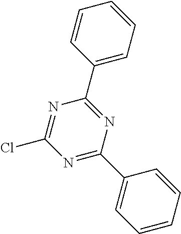 |

| Compound | F | Yield |
|---|---|---|
| 2-166 | 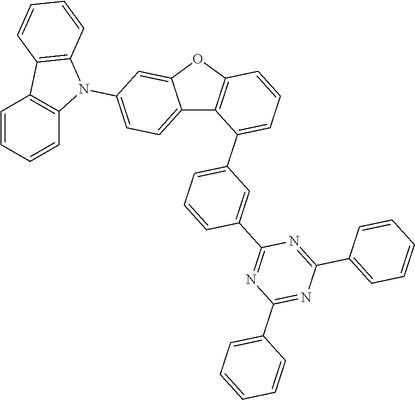 | 70% |
| 2-167 | 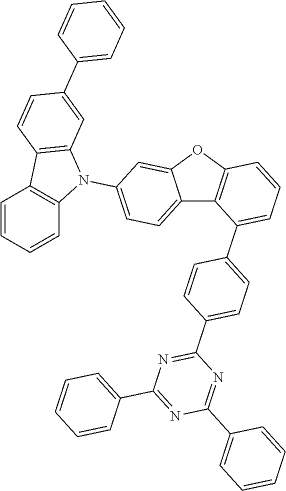 | 71% |
| 2-171 | 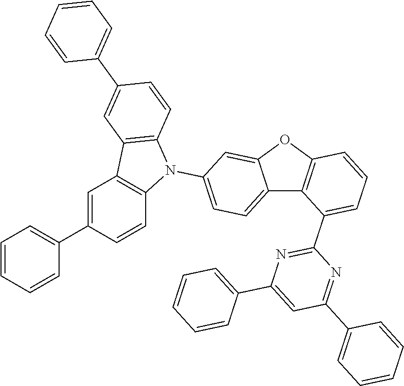 | 68% |
| 2-173 | 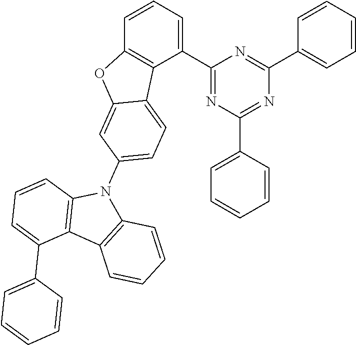 | 42% |
| 2-176 | 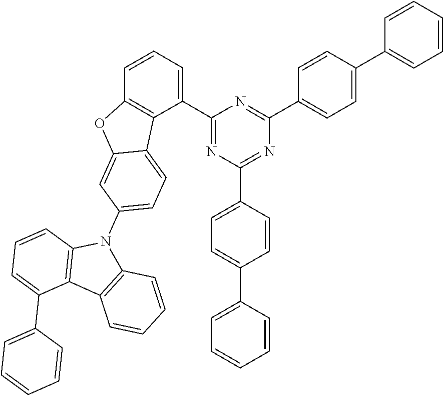 | 48% |
| 2-179 | 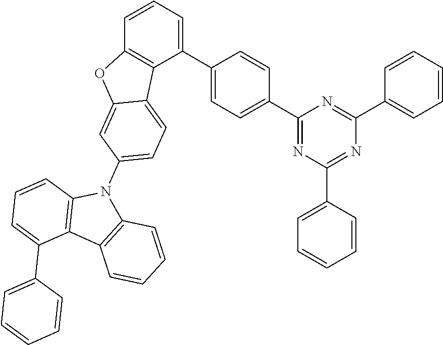 | 53% |

TABLE 12

| Compound | D | E |
|---|---|---|
| 2-181 | 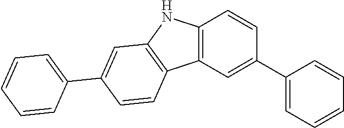 | 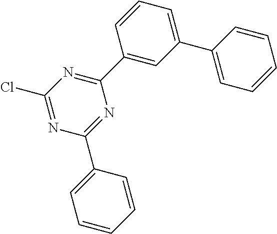 |
| 2-187 | 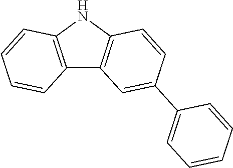 | 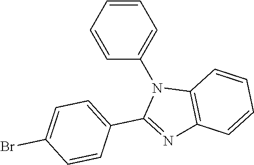 |
| 2-190 | 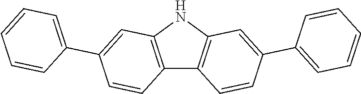 | 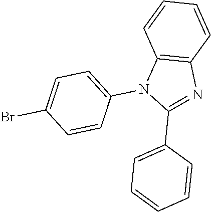 |
| 2-193 | 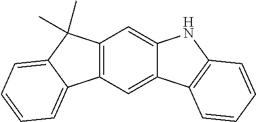 | 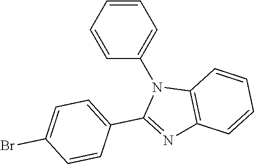 |
| 2-195 | 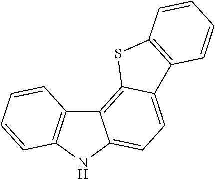 | 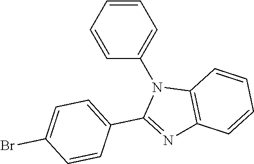 |
| 2-198 | 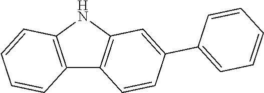 | 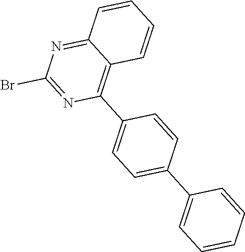 |

| Compound | F | Yield |
|---|---|---|
| 2-181 | 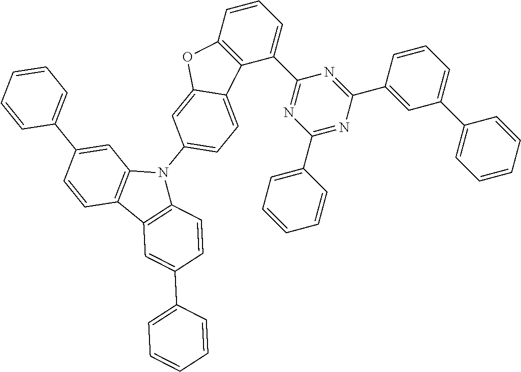 | 51% |
| 2-187 | 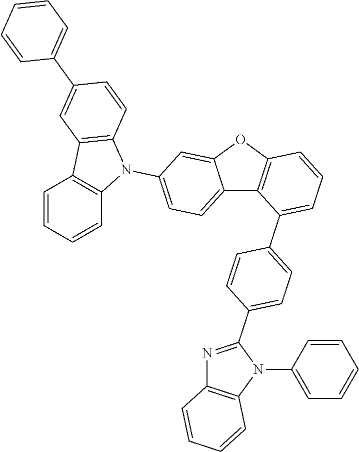 | 66% |
| 2-190 | 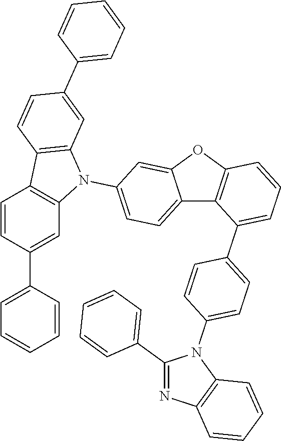 | 65% |
| 2-193 | 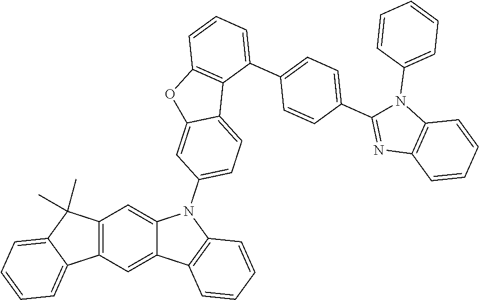 | 67% |
| 2-195 | 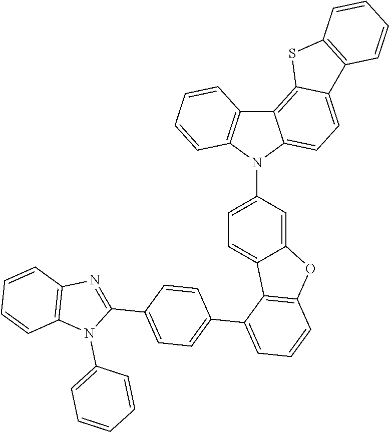 | 61% |
| 2-198 | 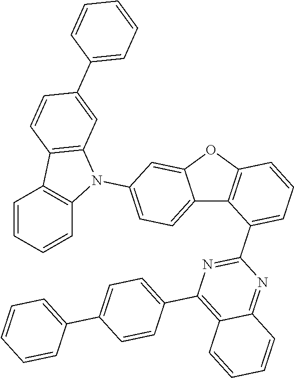 | 69% |

TABLE 13

| Compound | D | E |
|---|---|---|
| 2-199 | 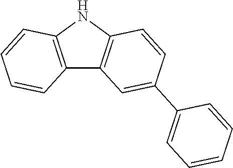 | 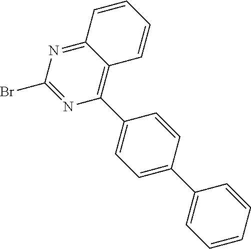 |
| 2-203 | 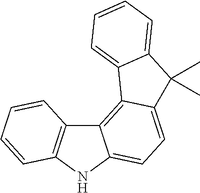 | 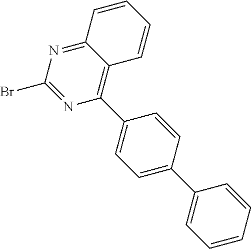 |
| 2-205 | 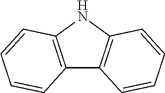 | 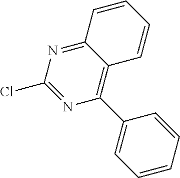 |
| 2-208 | 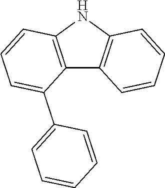 | 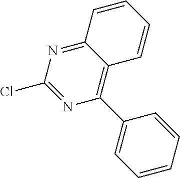 |
| 2-211 | 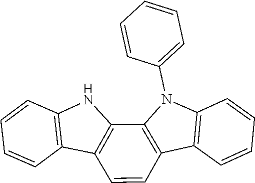 | 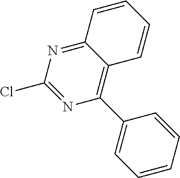 |
| 2-214 | 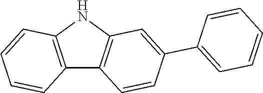 | 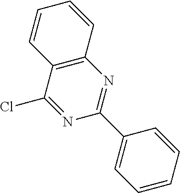 |

| Compound | F | Yield |
|---|---|---|
| 2-199 | 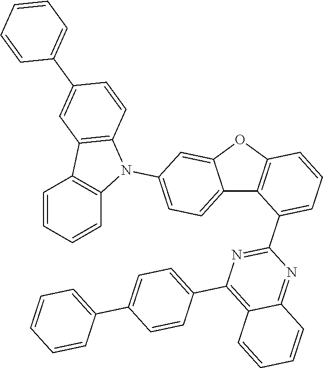 | 63% |
| 2-203 | 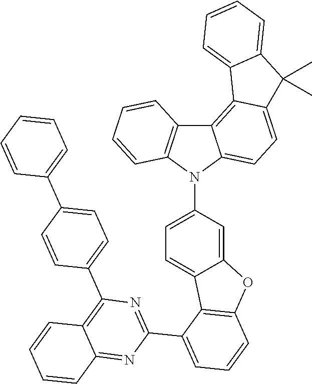 | 64% |
| 2-205 | 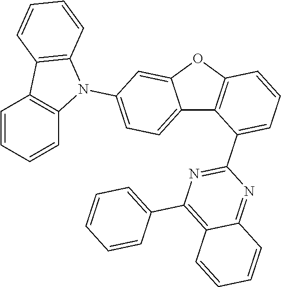 | 64% |
| 2-208 | 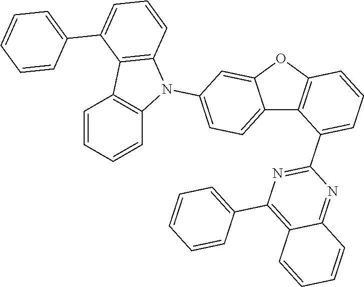 | 70% |
| 2-211 | 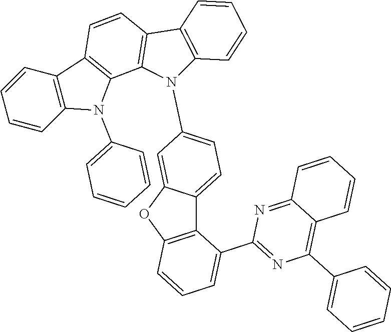 | 68% |
| 2-214 | 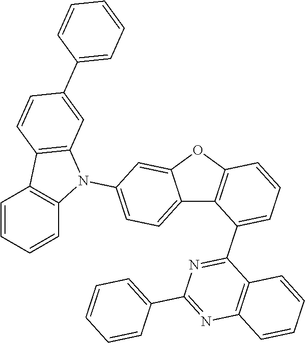 | 60% |

TABLE 14

| Compound | D | E |
|---|---|---|
| 2-218 | 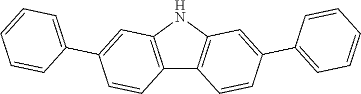 | 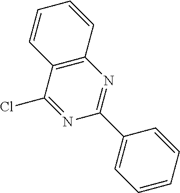 |
| 2-220 | 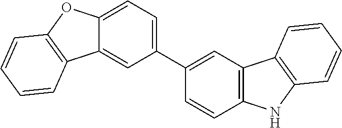 | 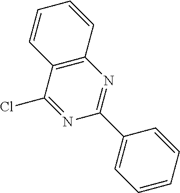 |
| 2-223 | 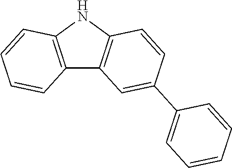 | 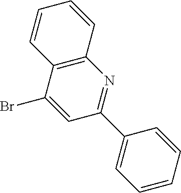 |
| 2-228 | 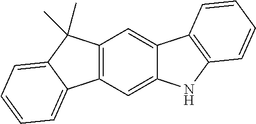 | 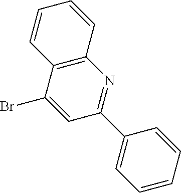 |
| 2-229 | 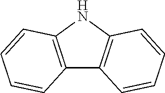 | 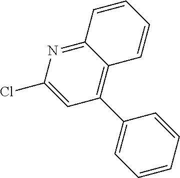 |
| 2-238 | 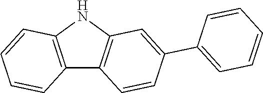 | 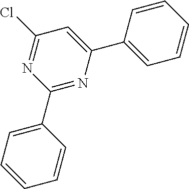 |

| Compound | F | Yield |
|---|---|---|
| 2-218 | 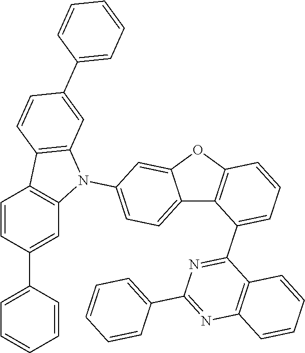 | 66% |
| 2-220 | 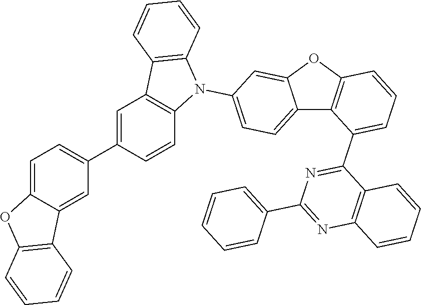 | 61% |
| 2-223 | 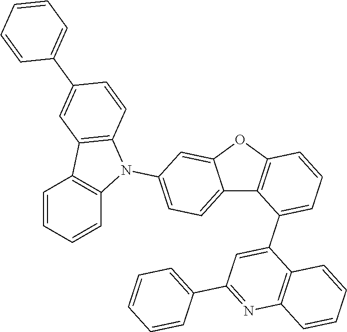 | 51% |
| 2-228 | 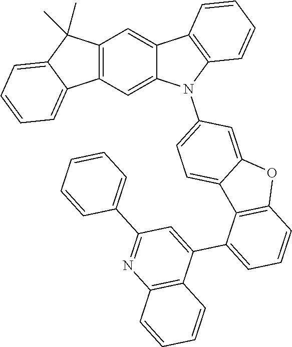 | 54% |
| 2-229 | 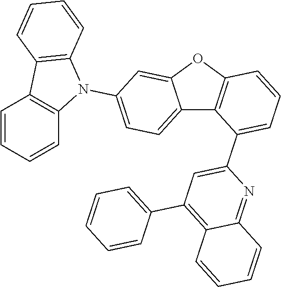 | 49% |
| 2-238 | 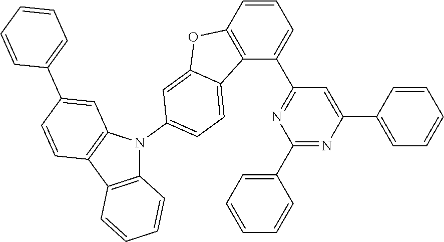 | 54% |

TABLE 15

| Compound | D | E |
|---|---|---|
| 2-241 | 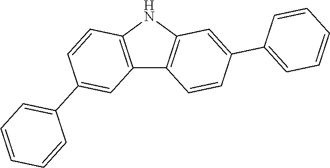 | 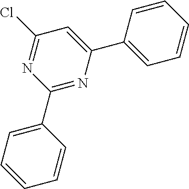 |
| 2-246 | 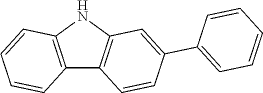 | 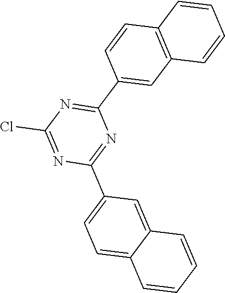 |
| 2-247 | 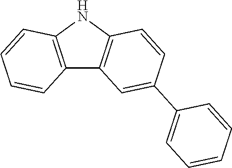 | 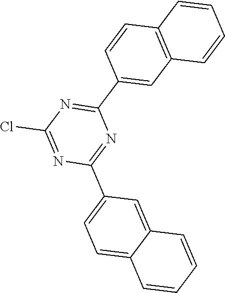 |
| 2-249 | 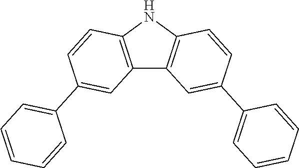 | 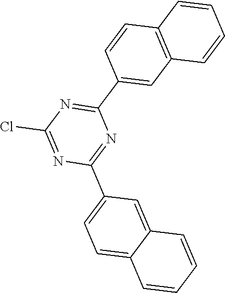 |
| 2-253 | 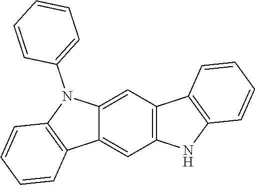 | 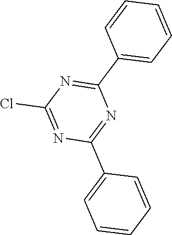 |
| 2-255 | 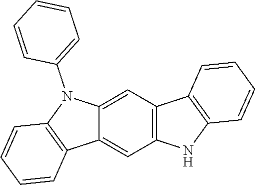 | 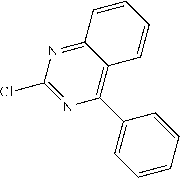 |
| 2-263 | 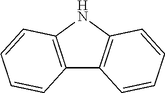 | 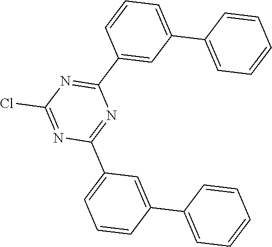 |
| 2-264 | 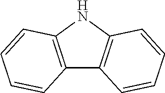 | 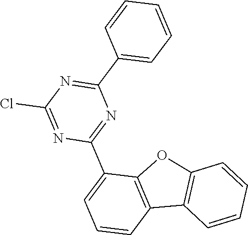 |
| 2-265 | 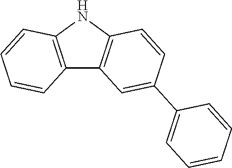 | 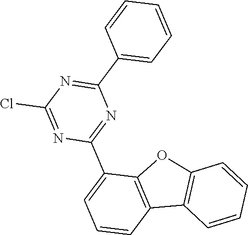 |
| 2-271 | 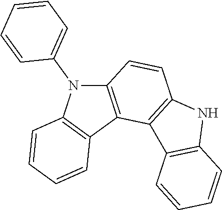 | 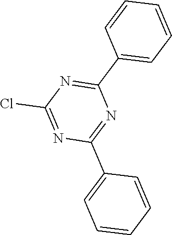 |
| 2-272 | 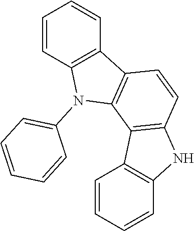 | 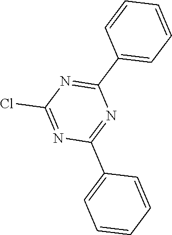 |
| 2-282 | 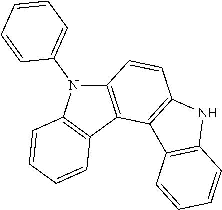 | 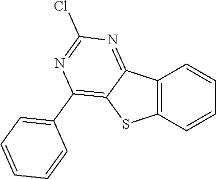 |
| 2-285 | 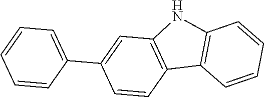 | 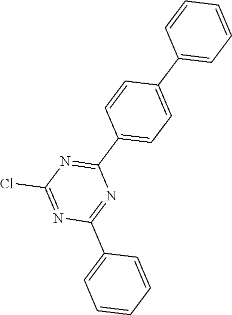 |

| Compound | F | Yield |
|---|---|---|
| 2-241 | 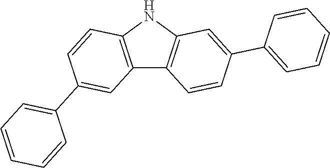 | 69% |
| 2-246 | 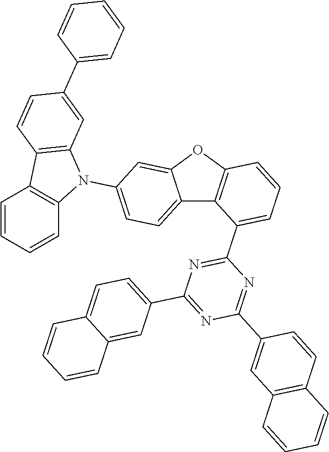 | 60% |
| 2-247 | 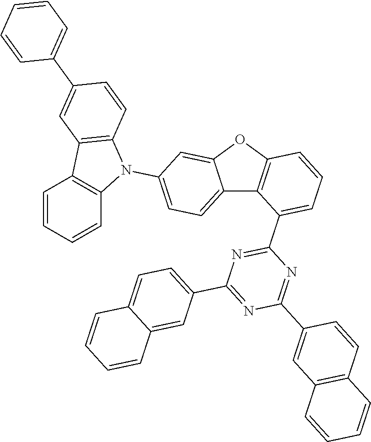 | 62% |
| 2-249 | 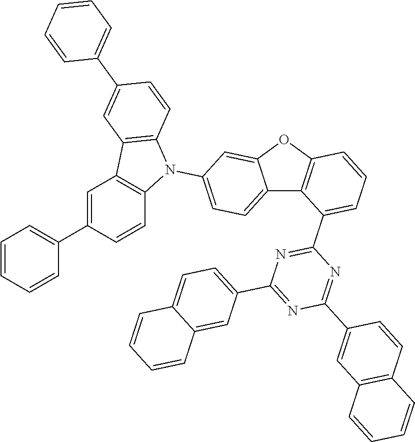 | 63% |
| 2-253 | 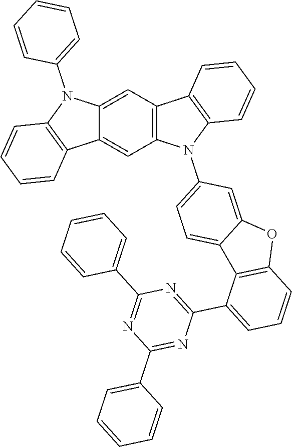 | 70% |
| 2-255 | 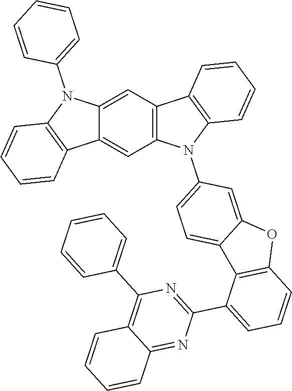 | 64% |
| 2-263 | 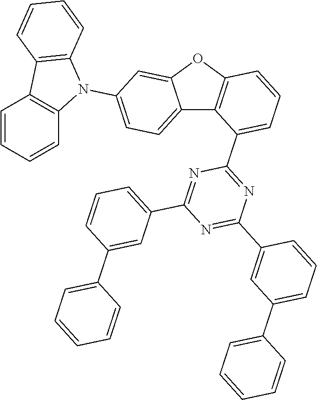 | 68% |
| 2-264 | 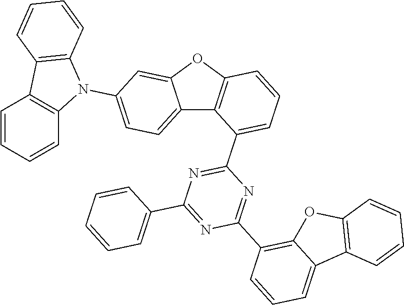 | 71% |
| 2-265 | 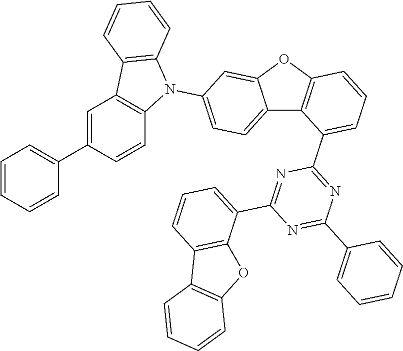 | 62% |
| 2-271 | 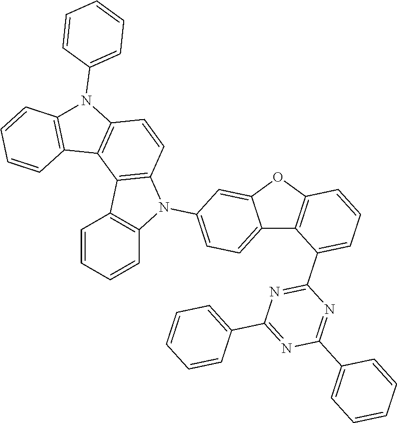 | 60% |
| 2-272 | 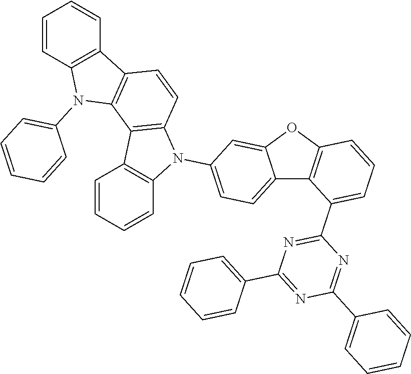 | 67% |
| 2-282 | 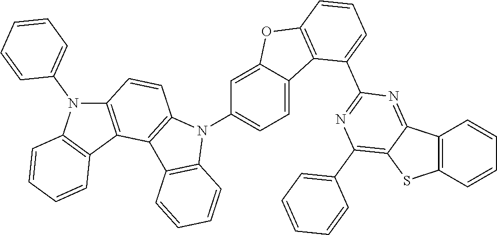 | 62% |
| 2-285 | 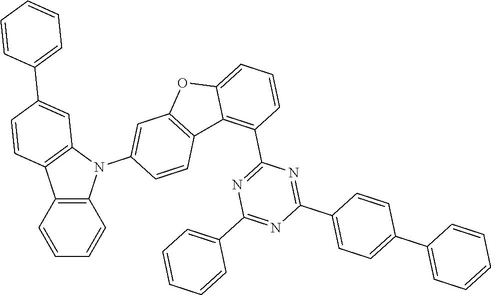 | 68% |

A compound other than the compounds shown in Tables 2 to 15 among Compounds 2-1 to 2-436 was also prepared in the same manner as in the method described in the above-described Preparation Examples.

The synthetic confirmation data of the compounds prepared above are as shown in the following Tables 16 and 17.

TABLE 16

| Compound | FD-Mass | Compound | FD-Mass |
|---|---|---|---|
| 1-1 | m/z = 650.76(C48H30N2O = 650.24) | 1-2 | m/z = 650.76(C48H30N2O = 650.24) |
| 1-3 | m/z = 700.82(C52H32N2O = 700.25) | 1-4 | m/z = 700.82(C52H32N2O = 700.25) |
| 1-5 | m/z = 700.82(C52H32N2O = 700.25) | 1-6 | m/z = 700.82(C52H32N2O = 700.25) |
| 1-7 | m/z = 726.86(C54H34N2O = 726.27) | 1-8 | m/z = 726.86(C54H34N2O = 726.27) |
| 1-9 | m/z = 726.86(C54H34N2O = 726.27) | 1-10 | m/z = 726.86(C54H34N2O = 726.27) |
| 1-11 | m/z = 726.86(C54H34N2O = 726.27) | 1-12 | m/z = 726.86(C54H34N2O = 726.27) |
| 1-13 | m/z = 726.86(C54H34N2O = 726.27) | 1-14 | m/z = 726.86(C54H34N2O = 726.27) |
| 1-15 | m/z = 726.86(C54H34N2O = 726.27) | 1-16 | m/z = 726.86(C54H34N2O = 726.27) |
| 1-17 | m/z = 726.86(C54H34N2O = 726.27) | 1-18 | m/z = 726.86(C54H34N2O = 726.27) |
| 1-19 | m/z = 669.88(C48H11D19N2O = 669.36) | 1-20 | m/z = 669.88(C48H11D19N2O = 669.36) |
| 1-21 | m/z = 661.83(C48H19D11N2O = 661.30) | 1-22 | m/z = 661.83(C48H19D11N2O = 661.30) |
| 1-23 | m/z = 680.95(C48D30N2O = 680.42) | 1-24 | m/z = 680.95(C48D30N2O = 680.42) |
| 1-25 | m/z = 650.76(C48H30N2O = 650.24) | 1-26 | m/z = 650.76(C48H30N2O = 650.24) |
| 1-27 | m/z = 700.82(C52H32N2O = 700.25) | 1-28 | m/z = 700.82(C52H32N2O = 700.25) |
| 1-29 | m/z = 700.82(C52H32N2O = 700.25) | 1-30 | m/z = 700.82(C52H32N2O = 700.25) |
| 1-31 | m/z = 726.86(C54H34N2O = 726.27) | 1-32 | m/z = 726.86(C54H34N2O = 726.27) |
| 1-33 | m/z = 726.86(C54H34N2O = 726.27) | 1-34 | m/z = 726.86(C54H34N2O = 726.27) |
| 1-35 | m/z = 726.86(C54H34N2O = 726.27) | 1-36 | m/z = 726.86(C54H34N2O = 726.27) |
| 1-37 | m/z = 726.86(C54H34N2O = 726.27) | 1-38 | m/z = 726.86(C54H34N2O = 726.27) |
| 1-39 | m/z = 726.86(C54H34N2O = 726.27) | 1-40 | m/z = 726.86(C54H34N2O = 726.27) |
| 1-41 | m/z = 726.86(C54H34N2O = 726.27) | 1-42 | m/z = 726.86(C54H34N2O = 726.27) |
| 1-43 | m/z = 669.88(C48H11D19N2O = 669.36) | 1-44 | m/z = 669.88(C48H11D19N2O = 669.36) |
| 1-45 | m/z = 661.83(C48H19D11N2O = 661.30) | 1-46 | m/z = 661.83(C48H19D11N2O = 661.30) |
| 1-47 | m/z = 680.95(C48D30N2O = 680.42) | 1-48 | m/z = 680.95(C48D30N2O = 680.42) |
| 1-49 | m/z = 650.76(C48H30N2O = 650.24) | 1-50 | m/z = 650.76(C48H30N2O = 650.24) |
| 1-51 | m/z = 700.82(C52H32N2O = 700.25) | 1-52 | m/z = 700.82(C52H32N2O = 700.25) |
| 1-53 | m/z = 700.82(C52H32N2O = 700.25) | 1-54 | m/z = 700.82(C52H32N2O = 700.25) |
| 1-55 | m/z = 726.86(C54H34N2O = 726.27) | 1-56 | m/z = 726.86(C54H34N2O = 726.27) |
| 1-57 | m/z = 726.86(C54H34N2O = 726.27) | 1-58 | m/z = 726.86(C54H34N2O = 726.27) |
| 1-59 | m/z = 726.86(C54H34N2O = 726.27) | 1-60 | m/z = 726.86(C54H34N2O = 726.27) |
| 1-61 | m/z = 726.86(C54H34N2O = 726.27) | 1-62 | m/z = 726.86(C54H34N2O = 726.27) |
| 1-63 | m/z = 726.86(C54H34N2O = 726.27) | 1-64 | m/z = 726.86(C54H34N2O = 726.27) |
| 1-65 | m/z = 726.86(C54H34N2O = 726.27) | 1-66 | m/z = 726.86(C54H34N2O = 726.27) |
| 1-67 | m/z = 669.88(C48H11D19N2O = 669.36) | 1-68 | m/z = 669.88(C48H11D19N2O = 669.36) |
| 1-69 | m/z = 661.83(C48H19D11N2O = 661.30) | 1-70 | m/z = 661.83(C48H19D11N2O = 661.30) |
| 1-71 | m/z = 680.95(C48D30N2O = 680.42) | 1-72 | m/z = 700.82(C52H32N2O = 700.25) |
| 1-73 | m/z = 650.76(C48H30N2O = 650.24) | 1-74 | m/z = 650.76(C48H30N2O = 650.24) |
| 1-75 | m/z = 700.82(C52H32N2O = 700.25) | 1-76 | m/z = 700.82(C52H32N2O = 700.25) |
| 1-77 | m/z = 700.82(C52H32N2O = 700.25) | 1-78 | m/z = 700.82(C52H32N2O = 700.25) |
| 1-79 | m/z = 726.86(C54H34N2O = 726.27) | 1-80 | m/z = 726.86(C54H34N2O = 726.27) |
| 1-81 | m/z = 726.86(C54H34N2O = 726.27) | 1-82 | m/z = 726.86(C54H34N2O = 726.27) |
| 1-83 | m/z = 726.86(C54H34N2O = 726.27) | 1-84 | m/z = 726.86(C54H34N2O = 726.27) |
| 1-85 | m/z = 726.86(C54H34N2O = 726.27) | 1-86 | m/z = 726.86(C54H34N2O = 726.27) |
| 1-87 | m/z = 726.86(C54H34N2O = 726.27) | 1-88 | m/z = 726.86(C54H34N2O = 726.27) |
| 1-89 | m/z = 726.86(C54H34N2O = 726.27) | 1-90 | m/z = 726.86(C54H34N2O = 726.27) |
| 1-91 | m/z = 669.88(C48H11D19N2O = 669.36) | 1-92 | m/z = 669.88(C48H11D19N2O = 669.36) |
| 1-93 | m/z = 661.83(C48H19D11N2O = 661.30) | 1-94 | m/z = 661.83(C48H19D11N2O = 661.30) |
| 1-95 | m/z = 680.95(C48D30N2O = 680.42) | 1-96 | m/z = 680.95(C48D30N2O = 680.42) |
| 1-97 | m/z = 650.76(C48H30N2O = 650.24) | 1-98 | m/z = 650.76(C48H30N2O = 650.24) |
| 1-99 | m/z = 700.82(C52H32N2O = 700.25) | 1-100 | m/z = 700.82(C52H32N2O = 700.25) |
| 1-101 | m/z = 700.82(C52H32N2O = 700.25) | 1-102 | m/z = 700.82(C52H32N2O = 700.25) |
| 1-103 | m/z = 726.86(C54H34N2O = 726.27) | 1-104 | m/z = 726.86(C54H34N2O = 726.27) |
| 1-105 | m/z = 726.86(C54H34N2O = 726.27) | 1-106 | m/z = 726.86(C54H34N2O = 726.27) |
| 1-107 | m/z = 726.86(C54H34N2O = 726.27) | 1-108 | m/z = 726.86(C54H34N2O = 726.27) |
| 1-109 | m/z = 726.86(C54H34N2O = 726.27) | 1-110 | m/z = 726.86(C54H34N2O = 726.27) |
| 1-111 | m/z = 726.86(C54H34N2O = 726.27) | 1-112 | m/z = 726.86(C54H34N2O = 726.27) |
| 1-113 | m/z = 726.86(C54H34N2O = 726.27) | 1-114 | m/z = 726.86(C54H34N2O = 726.27) |
| 1-115 | m/z = 669.88(C48H11D19N2O = 669.36) | 1-116 | m/z = 669.88(C48H11D19N2O = 669.36) |
| 1-117 | m/z = 661.83(C48H19D11N2O = 661.30) | 1-118 | m/z = 661.83(C48H19D11N2O = 661.30) |
| 1-119 | m/z = 680.95(C48D30N2O = 680.42) | 1-120 | m/z = 680.95(C48D30N2O = 680.42) |
| 1-121 | m/z = 650.76(C48H30N2O = 650.24) | 1-122 | m/z = 700.82(C52H32N2O = 700.25) |
| 1-123 | m/z = 700.82(C52H32N2O = 700.25) | 1-124 | m/z = 726.86(C54H34N2O = 726.27) |
| 1-125 | m/z = 726.86(C54H34N2O = 726.27) | 1-126 | m/z = 726.86(C54H34N2O = 726.27) |
| 1-127 | m/z = 726.86(C54H34N2O = 726.27) | 1-128 | m/z = 726.86(C54H34N2O = 726.27) |
| 1-129 | m/z = 726.86(C54H34N2O = 726.27) | 1-130 | m/z = 669.88(C48H11D19N2O = 669.36) |
| 1-131 | m/z = 661.83(C48H19D11N2O = 661.30) | 1-132 | m/z = 680.95(C48D30N2O = 680.42) |
| 1-133 | m/z = 650.76(C48H30N2O = 650.24) | 1-134 | m/z = 700.82(C52H32N2O = 700.25) |
| 1-135 | m/z = 700.82(C52H32N2O = 700.25) | 1-136 | m/z = 726.86(C54H34N2O = 726.27) |
| 1-137 | m/z = 726.86(C54H34N2O = 726.27) | 1-138 | m/z = 726.86(C54H34N2O = 726.27) |
| 1-139 | m/z = 726.86(C54H34N2O = 726.27) | 1-140 | m/z = 726.86(C54H34N2O = 726.27) |
| 1-141 | m/z = 726.86(C54H34N2O = 726.27) | 1-142 | m/z = 669.88(C48H11D19N2O = 669.36) |
| 1-143 | m/z = 661.83(C48H19D11N2O = 661.30) | 1-144 | m/z = 680.95(C48D30N2O = 680.42) |
| 1-145 | m/z = 650.76(C48H30N2O = 650.24) | 1-146 | m/z = 700.82(C52H32N2O = 700.25) |
| 1-147 | m/z = 700.82(C52H32N2O = 700.25) | 1-148 | m/z = 726.86(C54H34N2O = 726.27) |
| 1-149 | m/z = 726.86(C54H34N2O = 726.27) | 1-150 | m/z = 726.86(C54H34N2O = 726.27) |
| 1-151 | m/z = 726.86(C54H34N2O = 726.27) | 1-152 | m/z = 726.86(C54H34N2O = 726.27) |
| 1-153 | m/z = 726.86(C54H34N2O = 726.27) | 1-154 | m/z = 669.88(C48H11D19N2O = 669.36) |
| 1-155 | m/z = 661.83(C48H19D11N2O = 661.30) | 1-156 | m/z = 726.86(C54H34N2O = 726.27) |
| 1-157 | m/z = 650.76(C48H30N2O = 650.24) | 1-158 | m/z = 700.82(C52H32N2O = 700.25) |
| 1-159 | m/z = 700.82(C52H32N2O = 700.25) | 1-160 | m/z = 726.86(C54H34N2O = 726.27) |
| 1-161 | m/z = 726.86(C54H34N2O = 726.27) | 1-162 | m/z = 726.86(C54H34N2O = 726.27) |
| 1-163 | m/z = 726.86(C54H34N2O = 726.27) | 1-164 | m/z = 726.86(C54H34N2O = 726.27) |
| 1-165 | m/z = 726.86(C54H34N2O = 726.27) | 1-166 | m/z = 669.88(C48H11D19N2O = 669.36) |
| 1-167 | m/z = 661.83(C48H19D11N2O = 661.30) | 1-168 | m/z = 680.95(C48D30N2O = 680.42) |
| 1-169 | m/z = 650.76(C48H30N2O = 650.24) | 1-170 | m/z = 700.82(C52H32N2O = 700.25) |
| 1-171 | m/z = 700.82(C52H32N2O = 700.25) | 1-172 | m/z = 726.86(C54H34N2O = 726.27) |
| 1-173 | m/z = 726.86(C54H34N2O = 726.27) | 1-174 | m/z = 726.86(C54H34N2O = 726.27) |
| 1-175 | m/z = 726.86(C54H34N2O = 726.27) | 1-176 | m/z = 726.86(C54H34N2O = 726.27) |
| 1-177 | m/z = 726.86(C54H34N2O = 726.27) | 1-178 | m/z = 669.88(C48H11D19N2O = 669.36) |
| 1-179 | m/z = 661.83(C48H19D11N2O = 661.30) | 1-180 | m/z = 680.95(C48D30N2O = 680.42) |
| 2-1 | m/z = 564.63(C39H24N4O = 564.20) | 2-2 | m/z = 640.73(C45H28N4O = 640.23) |
| 2-3 | m/z = 640.73(C45H28N4O = 640.23) | 2-4 | m/z = 716.83(C51H32N4O = 717.26) |
| 2-5 | m/z = 716.83(C51H32N4O = 717.26) | 2-6 | m/z = 729.82(C51H31N5O = 729.25) |
| 2-7 | m/z = 729.82(C51H31N5O = 729.25) | 2-8 | m/z = 805.92(C57H35N5O = 805.28) |
| 2-9 | m/z = 730.81(C51H30N4O2 = 730.81) | 2-10 | m/z = 680.79(C48H32N4O = 680.26) |
| 2-11 | m/z = 680.79(C48H32N4O = 680.26) | 2-12 | m/z = 680.79(C48H32N4O = 680.26) |
| 2-13 | m/z = 670.78(C45H26N4OS = 670.18) | 2-14 | m/z = 654.71(C45H26N4O2 = 654.21) |
| 2-15 | m/z = 654.71(C45H26N4O2 = 654.21) | 2-16 | m/z = 670.78(C45H26N4OS = 670.18) |
| 2-17 | m/z = 640.73(C45H2N4O = 640.23) | 2-18 | m/z = 716.83(C51H32N4O = 716.26) |
| 2-19 | m/z = 716.83(C51H32N4O = 716.26) | 2-20 | m/z = 792.92(C57H36N4O = 792.29) |
| 2-21 | m/z = 792.92(C57H36N4O = 792.29) | 2-22 | m/z = 640.73(C45H2N4O = 640.23) |
| 2-23 | m/z = 792.92(C57H36N4O = 792.29) | 2-24 | m/z = 792.92(C57H36N4O = 792.29) |
| 2-25 | m/z = 792.92(C57H36N4O = 792.29) | 2-26 | m/z = 728.84(C52H32N4O = 728.26) |
| 2-27 | m/z = 728.84(C52H32N4O = 728.26) | 2-28 | m/z = 804.93(C58H36N4O = 804.29) |
| 2-29 | m/z = 746.21(C51H30N4OS = 746.21) | 2-30 | m/z = 756.89(C54H36N4O = 756.29) |
| 2-31 | m/z = 756.89(C54H36N4O = 756.29) | 2-32 | m/z = 679.81(C49H33N3O = 679.26) |
| 2-33 | m/z = 746.88(C51H30N4OS = 746.21) | 2-34 | m/z = 730.81(C51H30N4O2 = 730.24 |
| 2-35 | m/z = 730.81(C51H30N4O2 = 730.24 | 2-36 | m/z = 669.79(C46H27N3OS = 669.19) |
| 2-37 | m/z = 640.73(C45H28N4O = 640.23) | 2-38 | m/z = 640.73(C45H28N4O = 640.23) |
| 2-39 | m/z = 716.83(C51H32N4O = 717.26) | 2-40 | m/z = 716.83(C51H32N4O = 717.26) |
| 2-41 | m/z = 716.83(C51H43N4O = 716.26) | 2-42 | m/z = 716.83(C51H32N4O = 717.26) |
| 2-43 | m/z = 715.84(C52H33N3O = 715.26) | 2-44 | m/z = 715.84(C52H33N3O = 715.26) |
| 2-45 | m/z = 640.73(C45H28N4O = 640.23 | 2-46 | m/z = 716.83(C51H32N4O = 716.26) |
| 2-47 | m/z = 716.83(C51H32N4O = 716.26) | 2-48 | m/z = 792.92(C57H36N4O = 792.29) |
| 2-49 | m/z = 756.89(C54H36N4O = 756.29) | 2-50 | m/z = 716.83(C51H32N4O = 716.26) |
| 2-51 | m/z = 716.83(C51H32N4O = 716.26) | 2-52 | m/z = 716.83(C51H32N4O = 716.26) |
| 2-53 | m/z = 792.92(C57H36N4O = 792.29) | 2-54 | m/z = 792.92(C57H36N4O = 792.29) |
| 2-55 | m/z = 601.69(C43H27N3O = 601.69) | 2-56 | m/z = 601.69(C43H27N3O = 601.69) |
| 2-57 | m/z = 677.79(C49H31N3O = 677.25) | 2-58 | m/z = 677.79(C49H31N3O = 677.25) |
| 2-59 | m/z = 677.79(C49H31N3O = 677.25) | 2-60 | m/z = 677.79(C49H31N3O = 677.25) |
| 2-61 | m/z = 753.89(C55H35N3O = 753.28) | 2-62 | m/z = 753.89(C55H35N3O = 753.28) |
| 2-63 | m/z = 753.89(C55H35N3O = 753.28) | 2-64 | m/z = 753.89(C55H35N3O = 753.28) |
| 2-65 | m/z = 717.85(C52H35N3O = 717.28) | 2-66 | m/z = 717.85(C52H35N3O = 717.28) |
| 2-67 | m/z = 707.84(C49H29N3OS = 707.20) | 2-68 | m/z = 691.77(C49H29N3O2 = 691.23) |
| 2-69 | m/z = 613.70(C44H27N3O = 613.22) | 2-70 | m/z = 689.80(C50H31N3O = 689.25) |
| 2-71 | m/z = 689.80(C50H31N3O = 689.25) | 2-72 | m/z = 689.80(C50H31N3O = 689.25) |
| 2-73 | m/z = 765.90(C56H35N3O = 765.28) | 2-74 | m/z = 765.90(C56H35N3O = 765.28) |
| 2-75 | m/z = 729.86(C53H35N3O = 729.28) | 2-76 | m/z = 719.20(C50H29N3OS = 719.20) |
| 2-77 | m/z = 537.61(C38H23N3O = 537.18) | 2-78 | m/z = 613.70(C44H27N3O = 613.22) |
| 2-79 | m/z = 613.70(C44H27N3O = 613.22) | 2-80 | m/z = 613.70(C44H27N3O = 613.22) |
| 2-81 | m/z = 689.80(C50H31N3O = 689.25) | 2-82 | m/z = 689.80(C50H31N3O = 689.25) |
| 2-83 | m/z = 702.80(C50H30N4O = 702.24) | 2-84 | m/z = 702.80(C50H30N4O = 702.24) |
| 2-85 | m/z = 537.61(C38H23N3O = 537.18) | 2-86 | m/z = 613.70(C44H27N3O = 613.22) |
| 2-87 | m/z = 613.70(C44H27N3O = 613.22) | 2-88 | m/z = 613.70(C44H27N3O = 613.22) |
| 2-89 | m/z = 689.80(C50H31N3O = 689.25) | 2-90 | m/z = 689.80(C50H31N3O = 689.25) |
| 2-91 | m/z = 778.90(C56H34N4O = 78.27) | 2-92 | m/z = 703.78(C50H29N3O2 = 703.23) |
| 2-93 | m/z = 536.62(C39H24N2O = 536.19) | 2-94 | m/z = 612.72(C45H28N2O = 612.22) |
| 2-95 | m/z = 612.72(C45H28N2O = 612.22) | 2-96 | m/z = 612.72(C45H28N2O = 612.22) |
| 2-97 | m/z = 688.81(C51H32N2O = 688.25) | 2-98 | m/z = 688.81(C51H32N2O = 688.25) |
| 2-99 | m/z = 652.78(C48H32N2O = 652.25) | 2-100 | m/z = 652.78(C48H32N2O = 652.25) |
| 2-101 | m/z = 536.62(C39H24N2O = 536.19) | 2-102 | m/z = 612.72(C45H28N2O = 612.22) |
| 2-103 | m/z = 612.72(C45H28N2O = 612.22) | 2-104 | m/z = 612.72(C45H28N2O = 612.22) |
| 2-105 | m/z = 688.81(C51H32N2O = 688.25) | 2-106 | m/z = 688.81(C51H32N2O = 688.25) |
| 2-107 | m/z = 642.77(C45H26N2OS = 642.18) | 2-108 | m/z = 626.70(C45H27N2O2 = 626.20) |
| 2-109 | m/z = 563.65(C40H25N3O = 563.20) | 2-110 | m/z = 639.73(C46H29N3O = 639.23) |
| 2-111 | m/z = 639.73(C46H29N3O = 639.23) | 2-112 | m/z = 715.84(C52H33N3O = 715.26) |
| 2-113 | m/z = 715.84(C52H33N3O = 715.26) | 2-114 | m/z = 715.84(C52H33N3O = 715.26) |
| 2-115 | m/z = 639.74(C46H29N3O = 639.23) | 2-116 | m/z = 715.84(C52H33N3O = 715.26) |
| 2-117 | m/z = 664.75(C47H28N4O = 664.23) | 2-118 | m/z = 740.85(C53H32N4O = 740.26) |
| 2-119 | m/z = 740.85(C53H32N4O = 740.26) | 2-120 | m/z = 740.85(C53H32N4O = 740.26) |
| 2-121 | m/z = 816.94(C59H36N4O = 816.29) | 2-122 | m/z = 816.94(C59H36N4O = 816.29) |
| 2-123 | m/z = 829.94(C59H35N5O = 829.28) | 2-124 | m/z = 829.94(C59H35N5O = 829.28) |
| 2-125 | m/z = 729.82(C51H31N5O = 729.25) | 2-126 | m/z = 805.92(C57H35N5O = 805.28) |
| 2-127 | m/z = 702.80(C50H30N4O = 702.24) | 2-128 | m/z = 766.27(C55H34N4O = 766.27) |
| 2-129 | m/z = 564.63(C39H24N4O = 564.20) | 2-130 | m/z = 640.73(C45H28N4O = 640.23) |
| 2-131 | m/z = 640.73(C45H28N4O = 640.23) | 2-132 | m/z = 716.83(C51H32N4O = 717.26) |
| 2-133 | m/z = 716.83(C51H32N4O = 717.26) | 2-134 | m/z = 729.82(C51H31N5O = 729.25) |
| 2-135 | m/z = 729.82(C51H31N5O = 729.25) | 2-136 | m/z = 805.92(C57H35N5O = 805.28) |
| 2-137 | m/z = 730.81(C51H30N4O2 = 730.81) | 2-138 | m/z = 680.79(C48H32N4O = 680.26) |
| 2-139 | m/z = 680.79(C48H32N4O = 680.26) | 2-140 | m/z = 680.79(C48H32N4O = 680.26) |
| 2-141 | m/z = 670.78(C45H26N4OS = 670.18) | 2-142 | m/z = 654.71(C45H26N4O2 = 654.21) |
| 2-143 | m/z = 654.71(C45H26N4O2 = 654.21) | 2-144 | m/z = 670.78(C45H26N4OS = 670.18) |
| 2-145 | m/z = 640.73(C45H2N4O = 640.23) | 2-146 | m/z = 716.83(C51H32N4O = 716.26) |
| 2-147 | m/z = 716.83(C51H32N4O = 716.26) | 2-148 | m/z = 792.92(C57H36N4O = 792.29) |
| 2-149 | m/z = 792.92(C57H36N4O = 792.29) | 2-150 | m/z = 640.73(C45H2N4O = 640.23) |
| 2-151 | m/z = 792.92(C57H36N4O = 792.29) | 2-152 | m/z = 792.92(C57H36N4O = 792.29) |
| 2-153 | m/z = 792.92(C57H36N4O = 792.29) | 2-154 | m/z = 728.84(C52H32N4O = 728.26) |
| 2-155 | m/z = 728.84(C52H32N4O = 728.26) | 2-156 | m/z = 804.93(C58H36N4O = 804.29) |
| 2-157 | m/z = 746.21(C51H30N4OS = 746.21) | 2-158 | m/z = 756.89(C54H36N4O = 756.29) |
| 2-159 | m/z = 756.89(C54H36N4O = 756.29) | 2-160 | m/z = 679.81(C49H33N3O = 679.26) |
| 2-161 | m/z = 746.88(C51H30N4OS = 746.21) | 2-162 | m/z = 730.81(C51H30N4O2 = 730.24 |
| 2-163 | m/z = 730.81(C51H30N4O2 = 730.24 | 2-164 | m/z = 669.79(C46H27N3OS = 669.19) |
| 2-165 | m/z = 640.73(C45H28N4O = 640.23) | 2-166 | m/z = 640.73(C45H28N4O = 640.23) |
| 2-167 | m/z = 716.83(C51H32N4O = 717.26) | 2-168 | m/z = 716.83(C51H32N4O = 717.26) |
| 2-169 | m/z = 716.83(C51H43N4O = 716.26) | 2-170 | m/z = 716.83(C51H32N4O = 717.26) |
| 2-171 | m/z = 715.84(C52H33N3O = 715.26) | 2-172 | m/z = 715.84(C52H33N3O = 715.26) |
| 2-173 | m/z = 640.73(C45H28N4O = 640.23 | 2-174 | m/z = 716.83(C51H32N4O = 716.26) |
| 2-175 | m/z = 716.83(C51H32N4O = 716.26) | 2-176 | m/z = 792.92(C57H36N4O = 792.29) |
| 2-177 | m/z = 756.89(C54H36N4O = 756.29) | 2-178 | m/z = 716.83(C51H32N4O = 716.26) |
| 2-179 | m/z = 716.83(C51H32N4O = 716.26) | 2-180 | m/z = 716.83(C51H32N4O = 716.26) |
| 2-181 | m/z = 792.92(C57H36N4O = 792.29) | 2-182 | m/z = 792.92(C57H36N4O = 792.29) |
| 2-183 | m/z = 601.69(C43H27N3O = 601.69) | 2-184 | m/z = 601.69(C43H27N3O = 601.69) |
| 2-185 | m/z = 677.79(C49H31N3O = 677.25) | 2-186 | m/z = 677.79(C49H31N3O = 677.25) |
| 2-187 | m/z = 677.79(C49H31N3O = 677.25) | 2-188 | m/z = 677.79(C49H31N3O = 677.25) |
| 2-189 | m/z = 753.89(C55H35N3O = 753.28) | 2-190 | m/z = 753.89(C55H35N3O = 753.28) |
| 2-191 | m/z = 753.89(C55H35N3O = 753.28) | 2-192 | m/z = 753.89(C55H35N3O = 753.28) |
| 2-193 | m/z = 717.85(C52H35N3O = 717.28) | 2-194 | m/z = 717.85(C52H35N3O = 717.28) |
| 2-195 | m/z = 707.84(C49H29N3OS = 707.20) | 2-196 | m/z = 691.77(C49H29N3O2 = 691.23) |
| 2-197 | m/z = 613.70(C44H27N3O = 613.22) | 2-198 | m/z = 689.80(C50H31N3O = 689.25) |
| 2-199 | m/z = 689.80(C50H31N3O = 689.25) | 2-200 | m/z = 689.80(C50H31N3O = 689.25) |
| 2-201 | m/z = 765.90(C56H35N3O = 765.28) | 2-202 | m/z = 765.90(C56H35N3O = 765.28) |
| 2-203 | m/z = 729.86(C53H35N3O = 729.28) | 2-204 | m/z = 719.20(C50H29N3OS = 719.20) |
| 2-205 | m/z = 537.61(C38H23N3O = 537.18) | 2-206 | m/z = 613.70(C44H27N3O = 613.22) |
| 2-207 | m/z = 613.70(C44H27N3O = 613.22) | 2-208 | m/z = 613.70(C44H27N3O = 613.22) |
| 2-209 | m/z = 689.80(C50H31N3O = 689.25) | 2-210 | m/z = 689.80(C50H31N3O = 689.25) |
| 2-211 | m/z = 702.80(C50H30N4O = 702.24) | 2-212 | m/z = 702.80(C50H30N4O = 702.24) |
| 2-213 | m/z = 537.61(C38H23N3O = 537.18) | 2-214 | m/z = 613.70(C44H27N3O = 613.22) |
| 2-215 | m/z = 613.70(C44H27N3O = 613.22) | 2-216 | m/z = 613.70(C44H27N3O = 613.22) |
| 2-217 | m/z = 689.80(C50H31N3O = 689.25) | 2-218 | m/z = 689.80(C50H31N3O = 689.25) |
| 2-219 | m/z = 778.90(C56H34N4O = 78.27) | 2-220 | m/z = 703.78(C50H29N3O2 = 703.23) |
| 2-221 | m/z = 536.62(C39H24N2O = 536.19) | 2-222 | m/z = 612.72(C45H28N2O = 612.22) |
| 2-223 | m/z = 612.72(C45H28N2O = 612.22) | 2-224 | m/z = 612.72(C45H28N2O = 612.22) |
| 2-225 | m/z = 688.81(C51H32N2O = 688.25) | 2-226 | m/z = 688.81(C51H32N2O = 688.25) |
| 2-227 | m/z = 652.78(C48H32N2O = 652.25) | 2-228 | m/z = 652.78(C48H32N2O = 652.25) |
| 2-229 | m/z = 536.62(C39H24N2O = 536.19) | 2-230 | m/z = 612.72(C45H28N2O = 612.22) |
| 2-231 | m/z = 612.72(C45H28N2O = 612.22) | 2-232 | m/z = 612.72(C45H28N2O = 612.22) |
| 2-233 | m/z = 688.81(C51H32N2O = 688.25) | 2-234 | m/z = 688.81(C51H32N2O = 688.25) |
| 2-235 | m/z = 642.77(C45H26N2OS = 642.18) | 2-236 | m/z = 626.70(C45H27N2O2 = 626.20) |
| 2-237 | m/z = 563.65(C40H25N3O = 563.20) | 2-238 | m/z = 639.73(C46H29N3O = 639.23) |
| 2-239 | m/z = 639.73(C46H29N3O = 639.23) | 2-240 | m/z = 715.84(C52H33N3O = 715.26) |
| 2-241 | m/z = 715.84(C52H33N3O = 715.26) | 2-242 | m/z = 715.84(C52H33N3O = 715.26) |
| 2-243 | m/z = 639.74(C46H29N3O = 639.23) | 2-244 | m/z = 715.84(C52H33N3O = 715.26) |
| 2-245 | m/z = 664.75(C47H28N4O = 664.23) | 2-246 | m/z = 740.85(C53H32N4O = 740.26) |
| 2-247 | m/z = 740.85(C53H32N4O = 740.26) | 2-248 | m/z = 740.85(C53H32N4O = 740.26) |
| 2-249 | m/z = 816.94(C59H36N4O = 816.29) | 2-250 | m/z = 816.94(C59H36N4O = 816.29) |
| 2-251 | m/z = 829.94(C59H35N5O = 829.28) | 2-252 | m/z = 829.94(C59H35N5O = 829.28) |
| 2-253 | m/z = 729.82(C51H31N5O = 729.25) | 2-254 | m/z = 805.92(C57H35N5O = 805.28) |
| 2-255 | m/z = 702.80(C50H30N4O = 702.24) | 2-256 | m/z = 766.27(C55H34N4O = 766.27) |
| 2-257 | m/z = 716.83(C51H32N4O = 716.26) | 2-258 | m/z = 654.71(C45H26N4O2 = 654.21) |
| 2-259 | m/z = 730.81(C51H30N4O2 = 730.24) | 2-260 | m/z = 654.71(C45H26N4O2 = 654.21) |
| 2-261 | m/z = 730.81(C51H30N4O2 = 730.24) | 2-262 | m/z = 730.81(C51H30N4O2 = 730.24) |
| 2-263 | m/z = 716.83(C51H32N4O = 716.26) | 2-264 | m/z = 654.71(C45H26N4O2 = 654.21) |
| 2-265 | m/z = 730.81(C51H30N4O2 = 730.24) | 2-266 | m/z = 730.81(C51H30N4O2 = 730.24) |
| 2-267 | m/z = 746.88(C51H30N4OS = 741.21) | 2-268 | m/z = 756.89(C54H36N4O = 756.29) |
| 2-269 | m/z = 614.71(C43H26N4O = 614.21) | 2-270 | m/z = 664.77(C47H28N4O = 664.23) |
| 2-271 | m/z = 729.84(C51H31N5O = 729.25) | 2-272 | m/z = 729.84(C51H31N5O = 729.25) |
| 2-273 | m/z = 654.73(C45H26N4O2 = 654.21) | 2-274 | m/z = 670.79(C45H26N4OS = 670.18) |
| 2-275 | m/z = 779.90(C55H33N5O = 779.27) | 2-276 | m/z = 829.96(C59H35N5O = 829.28) |
| 2-277 | m/z = 704.79(C49H28N4O2 = 704.22) | 2-278 | m/z = 720.85(C49H28N4OS = 720.20) |
| 2-279 | m/z = 754.85(C53H30N4O2 = 754.24) | 2-280 | m/z = 770.91(C53H30N4OS = 770.21) |
| 2-281 | m/z = 693.82(C48H27N3OS = 693.19) | 2-282 | m/z = 758.90(C52H30N4OS = 758.21) |
| 2-283 | m/z = 799.97(C54H29N3OS2 = 799.18) | 2-284 | m/z = 783.90(C54H29N3O2S = 783.20) |
| 2-285 | m/z = 716.83(C51H32N4O = 717.26) | 2-286 | m/z = 716.83(C51H32N4O = 717.26) |
| 2-287 | m/z = 475.17 (C33H21N3O = 475.55) | 2-289 | m/z = 525.18 (C37H23N3O = 525.61) |
| 2-291 | m/z = 625.22 (C45H27N3O = 625.73) | 2-292 | m/z = 581.16 (C39H23N3OS = 581.69) |
| 2-295 | m/z = 565.18 (C39H23N3O2 = 565.63) | 2-300 | m/z = 591.23 (C42H29N3O = 591.71) |
| 2-305 | m/z = 640.23 (C45H28N4O = 640.75) | 2-311 | m/z = 551.20 (C39H25N3O = 551.65) |
| 2-314 | m/z = 777.28 (C57H35N3O = 777.93) | 2-315 | m/z = 701.25 (C51H31N3O = 701.83) |
| 2-316 | m/z = 777.28 (C57H35N3O = 777.93) | 2-319 | m/z = 716.26 (C51H32N4O = 716.84) |
| 2-327 | m/z = 715.23 (C51H29N3O2 = 715.81) | 2-333 | m/z = 807.23 (C57H33N3OS = 807.97) |
| 2-337 | m/z = 866.30 (C63H38N4O = 867.02) | 2-341 | m/z = 817.31 (C60H39N3O = 817.99) |
| 2-346 | m/z = 853.31 (C63H39N3O = 854.02) | 2-352 | m/z = 701.25 (C51H31N3O = 701.83) |

TABLE 17

| Compound | ¹H NMR(CDCl₃, 200 Mz) |
|---|---|
| 1-1 | δ = 8.55 (1H, d), 8.18 (1H, d), 8.12 (1H, d), |
| | 8.00 (1H, d), 7.94 (1H, d), 7.77~7.89 (5H, m), |
| | 7.25~7.63 (19H, m) |
| 1-2 | δ = 8.55 (1H, d), 8.18 (1H, d), 8.12 (1H, d), 8.00 |
| | (1H, d), 7.95 (1H, s), 7.94 (1H, d), 7.89 (1H, |
| | d), 7.77 (2H, s), 7.25~7.63 (20H, m) |
| 1-4 | δ = 8.55 (1H, d), 8.18 (1H, d), 8.12 (1H, d), |
| | 8.00 (3H, d), 7.94 (1H, d), 7.92 (1H, s), |
| | 7.25~7.89 (24H, m) |
| 1-7 | δ = 8.55 (1H, d), 8.18 (1H, d), 8.12 (1H, d), |
| | 7.77~8.00 (7H, m), 7.25~7.69 (24H, m) |
| 1-8 | δ = 8.55 (1H, d), 8.18 (1H, d), 8.12 (1H, d), |
| | 7.77~8.00 (7H, m), 7.25~7.89 (24H, m) |
| 1-12 | δ = 8.55 (1H, d), 8.18 (1H, d), 8.12 (1H, d), |
| | 7.77~7.95 (11H, m), 7.25~7.69 (20H, m) |
| 1-13 | δ = 8.55 (1H, d), 8.18 (1H, d), 8.12 (1H, d), |
| | 7.77~8.00 (9H, m), 7.63~7.69 (5H, m), 7.25~7.52 |
| | (17H, m) |
| 1-15 | δ = 8.55 (1H, d), 8.09~8.18 (3H, m), 7.69~8.00 |
| | (7H, m), 7.63~7.66 (3H, m), 7.25~7.52 (20H, m) |
| 1-17 | δ = 8.55 (1H, d), 8.18 (1H, d), 8.12 (1H, d), |
| | 8.00 (1H, d), 7.69~7.94 (7H, m), 7.63~7.69 (4H, |
| | m), 7.25~7.54 (19H, m) |
| 1-18 | δ = 8.52~8.52 (2H, d), 8.12~8.18 (2H, d), |
| | 7.69~8.00 (5H, m), 7.77~7.79 (3H, m), 7.63~7.89 |
| | (5H, m), 7.19~7.54 (18H, m) |
| 1-25 | δ = 8.49~8.55 (2H, d), 8.10~8.12 (2H, t), |
| | 7.69~7.94 (5H, m), 7.25~7.58 (21H, m) |
| 1-27 | δ = 8.42~8.55 (4H, d), 8.04~8.12 (4H, m), |
| | 7.69~7.94 (5H, m), 7.25~7.66 (19H, m) |
| 1-30 | δ = 8.49~8.55 (2H, d), 8.10~8.12 (2H, t), |
| | 7.87~8.00 (21H, m) |
| 1-32 | δ = 8.49~8.55 (2H, d), 8.10~8.12 (2H, t), |
| | 7.77~7.94 (4H, m), 7.25~7.66 (25H, m) |
| 1-34 | δ = 8.49~8.55 (2H, d), 8.10~8.12 (2H, t), |
| | 7.87~7.95 (4H, m), 7.77 (1H, s), 7.25~7.69(25H, m) |
| 1-37 | δ = 8.49~8.55 (2H, d), 8.10~8.12 (2H, t), |
| | 7.77~7.94 (7H, m), 7.62~7.69 (6H, m), 7.25~7.52 |
| | (17H, m) |
| 1-38 | δ = 8.49~8.55 (2H, d), 8.10~8.12 (2H, t), |
| | 7.87~7.94 (3H, m), 7.77~7.79 (3H, d), 7.62~7.69 |
| | (7H, m), 7.25~7.52 (16H, m) |
| 1-39 | δ = 8.49~8.55 (2H, d), 8.10~8.12 (2H, t), |
| | 7.77~7.94 (5H, m), 7.62~7.69 (4H, m), 7.25~7.52 |
| | (20H, m) |
| 1-40 | δ = 8.49~8.55 (2H, d), 8.09~8.12 (3H, m), |
| | 7.87~7.95 (4H, m), 7.77 (1H, s), 7.62~7.69 (5H, |
| | m), 7.25~7.52 (19H, m) |
| 1-41 | δ = 8.49~8.55 (2H, d), 8.10~8.12 (2H, t), |
| | 7.77~7.94 (6H, m), 7.62~7.69 (5H, m), 7.25~7.54 |
| | (19H, m) |
| 1-49 | δ = 8.55 (1H, d), 8.12~8.18 (3H, d), 7.89~8.00 |
| | (3H, m), 7.77~7.81 (3H, m), 7.25~7.66 (20H, m) |
| 1-50 | δ = 8.55 (1H, d), 8.12~8.18 (3H, d), 7.89~8.00 |
| | (4H, m), 7.77~7.79 (2H, d), 7.25~7.66 (20H, m) |
| 1-51 | δ = 8.55 (2H, d), 8.42 (1H, d), 7.89~8.18 (8H, |
| | m), 7.77~7.81 (3H, m), 7.25~7.66(18H, m) |
| 1-54 | δ = 8.55 (1H, d), 8.12~8.18 (3H, d), 7.89~8.00 |
| | (7H, m), 7.25~7.79 (21H, m) |
| 1-56 | δ = 8.55 (1H, d), 8.12~8.18 (3H, d), 7.89~8.00 |
| | (3H, m), 7.77~7.81 (3H, m), 7.25~7.77 (21H, m) |
| 1-57 | δ = 8.55 (1H, d), 8.12~8.18 (3H, d), 7.77~8.00 |
| | (10H, m), 7.25~7.66 (20H, m) |
| 1-59 | δ = 8.55 (1H, d), 8.12~8.18 (3H, d), 7.89~8.00 |
| | (4H, m), 7.77~7.79 (2H, d), 7.25~7.77 (24H, m) |
| 1-61 | δ = 8.55 (1H, d), 8.12~8.18 (3H, d), 7.89~8.00 |
| | (3H, m), 7.77~7.81 (5H, m), 7.92~7.68 (5H, m), |
| | 7.25~7.77 (17H, m) |
| 1-64 | δ = 8.55 (1H, d), 8.09~8.18 (4H, m), 7.89~8.00 |
| | (4H, m), 7.77~7.79 (2H, d), 7.63~7.66 (4H, m), |
| | 7.25~7.77 (19H, m) |
| 1-66 | δ = 8.55 (1H, d), 88.12~8.18 (3H, d), 7.89~8.00 |
| | (3H, m), 7.77~7.79 (2H, d), 7.62~7.68 (4H, m), |
| | 7.25~7.77 (17H, m) |
| 1-73 | δ = 8.49~8.55 (2H, d), 8.12~8.18 (3H, m), |
| | 7.89~7.94 (2H, m), 7.79~7.81 (2H, m), 7.25~7.66 |
| | (21H, m) |
| 1-74 | δ = 8.49~8.55 (2H, d), 8.10~8.18 (3H, m), |
| | 7.89~7.94 (2H, m), 7.79(1H, d), 7.25~7.66 (21H, m) |
| 1-78 | δ = 8.49~8.55 (2H, d), 8.10~8.18 (3H, m), |
| | 7.89~8.00 (6H, m), 7.25~7.79 (21H, m) |
| 1-80 | δ = 8.49~8.55 (2H, d), 8.10~8.18 (3H, m), |
| | 7.89~7.94 (2H, m), 7.79~7.81 (2H, m), 7.25~7.66 |
| | (25H, m) |
| 1-81 | δ = 8.49~8.55 (2H, d), 8.10~8.18 (3H, m), |
| | 7.79~7.94 (8H, m), 7.25~7.66 (21H, m) |
| 1-83 | δ = 8.49~8.55 (2H, d), 8.10~8.18 (3H, m), |
| | 7.89~7.95 (3H, m), 7.79 (1H, d), 7.25~7.66 (25H, m) |
| 1-85 | δ = 8.49~8.55 (2H, d), 8.10~8.18 (3H, m), |
| | 7.89~7.94 (2H, m), 7.79~7.81 (4H, m), 7.62~7.68 |
| | (6H, m), 7.25~7.52 (17H, m) |
| 1-88 | δ = 8.49~8.55 (2H, d), 8.09~8.18 (4h, m), |
| | 7.89~7.95 (3H, m), 7.79 (1H, d), 7.62~7.89 (5H, |
| | m), 7.25~7.52 (19H, m) |
| 1-90 | δ = 8.49~8.55 (2H, d), 8.10~8.18 (3H, m), |
| | 7.89~7.95 (3H, m), 7.79 (2H, d), 7.25~7.68 (24H, m) |
| 1-97 | δ = 8.55 (1H, d), 8.10~8.12 (2H, t), 7.89~7.90 |
| | (2H, m), 7.79~7.81 (2H, m), 7.25~7.66 (23H, m) |
| 1-98 | δ = 8.55 (1H, d), 8.10~8.12 (2H, t), 7.89~7.94 |
| | (4H, m), 7.79 (1H, d), 7.25~7.66 (22H, m) |
| 1-102 | δ = 8.55 (1H, d), 8.10~8.12 (2H, t), 7.89~7.95 |
| | (7H, m), 7.25~7.79 (23H, m) |
| 1-107 | δ = 8.55 (1H, d), 8.10~8.12 (2H, t), 7.89~7.95 |
| | (4H, m), 7.79 (1H, d), 7.25~7.66 (26H, m) |
| 1-109 | δ = 8.55 (1H, d), 8.10~8.12 (2H, t), 7.89~7.94 |
| | (H, m), 7.79~7.81 (4H, m), 7.25~7.68 (24H, m) |
| 1-111 | δ = 8.55 (1H, d), 8.09~8.12 (3H, m), 7.89~7.94 |
| | (3H, m), 7.79~7.81 (2H, m), 7.25~7.66 (25H, m) |
| 1-112 | δ = 8..55 (1H, d), 8.09~8.12 (3H, m), 7.89~7.95 |
| | (4H, m), 7.79 (1H, d), 7.25~7.66 (25H, m) |
| 1-113 | δ = 8.55 (1H, d), 8.10~8.12 (2H, t), 7.89~7.94 |
| | (3H, m), 7.79~7.81 (3H, m), 7.25~7.68 (25H, m) |
| 1-114 | δ = 8.55 (1H, d), 8.10~8.12 (2H, t), 7.89~7.95 |
| | (4H, m), 7.79 (2H, d), 7.25~7.68 (25H, m) |
| 1-122 | δ = 8.55 (1H, d), 8.42 (1H, d), 7.87~8.12 (1H, |
| | m), 7.25~7.77 (20H, m) |
| 1-123 | δ = 8.55 (1H, d), 8.12~8.18 (2H, d), 7.87~8.02 |
| | (8H, m), 7.25~7.77 (21H, m) |
| 1-124 | δ = 8.55 (1H, d), 8.18 (1H, d), 8.15 (1H, d), |
| | 7.87~8.02 (5H, m), 7.25~7.77 (26H, m) |
| 1-125 | δ = 8.55 (1H, d), 8.18 (1H, d), 8.12 (1H, d), |
| | 7.87~8.02 (5H, m), 7.25~7.77 (27H, m) |
| 1-127 | δ = 8.55 (1H, d), 8.8 (1H, d), 8.12 (1H, d), |
| | 7.87~8.02 (5H, m), 7.63~7.79 (9H, m), 7.25~7.52 |
| | (16H, m) |
| 1-128 | δ = 8.55 (1H, d), 8.09~8.18 (3H, m), 7.87~8.02 |
| | (5H, m), 7.63~7.77 (6H, m), 7.25~7.52 (19H, m) |
| 1-129 | δ = 8.55 (1H, d), 8.18 (1H, d), 8.12 (1H, d), |
| | 7.87~8.02 (5H, m), 7.63~7.79 (8H, m), 7.25~7.54 |
| | (18H, m) |
| 1-133 | δ = 8.55 (1H, d), 8.49 (1H, d), 8.10~8.12 (2H, |
| | t), 8.02 (1H, s), 7.87~7.94 (3H, m), 7.25~7.77 |
| | (23H, m) |
| 1-148 | δ = 8.55 (1H, d), 8.18 (2H, d), 8.12 (1H, d), |
| | 7.89~8.02 (4H, m), 7.25~7.79 (26H, m) |
| 1-149 | δ = 8.55 (1H, d), 8.18 (2H, d), 8.12 (1H, d), |
| | 7.89~8.02 (4H, m), 7.25~7.77 (26H, m) |
| 1-150 | δ = 8.55 (1H, d), 8.18 (2H, d), 8.12 (1H, d), |
| | 8.00~8.02 (2H, m), 7.25~7.7.89 (28H, m) |
| 1-151 | δ = 8.55 (1H, d), 8.18 (2H, d), 8.12 (1H, d), |
| | 7.89~8.02 (4H, m), 7.66~7.79 (10H, m), 7.25~7.52 |
| | (16H, m) |
| 1-152 | δ = 8.55 (1H, d), 8.18 (2H, d), 8.12 (1H, d), |
| | 7.89~8.02 (4H, m), 7.77~7.79 (2H, d), 7.62~7.63 |
| | (2H, m), 7.25~7.52 (19H, m) |
| 1-155 | δ = 8.55 (1H, d), 8.18 (2H, d), 8.12 (1H, d), |
| | 7.94~8.00 (2H, d), 7.77~7.79 (2H, d), 7.45~7.63 |
| | (8H, m), 7.25~7.33 (3H, m) |
| 1-156 | δ = — |
| 1-159 | δ = 8.55 (1H, d), 8.49 (1H, d), 8.18~8.10 (3H, |
| | m), 7.89~8.02 (6H, m), 7.25~7.79 (21H, m) |
| 1-163 | δ = 8.55 (1H, d), 8.49 (1H, d), 8.18~8.10 (3H, |
| | m), 8.02 (1H, s), 7.89~7.94 (2H, m), 7.79 (3H, |
| | d), 7.62~7.72 (7H, m), 7.25~7.52 (16H, m) |
| 1-167 | δ = 8.55 (1H, d), 8.49 (1H, d), 8.10~8.18 (3H, m), |
| | 7.94 (1H, d), 7.79 (1H, d), 7.45~7.63 (8H, m), |
| | 7.25~7.33 (3H, m) |
| 1-170 | δ = 8.55 (1H, d), 8.42 (1H, d), 8.02~8.12 (5H, |
| | m), 7.89~7.90 (3H, m), 7.25~7.72 (21H, m) |
| 1-171 | δ = 8.55 (1H, d), 7.79~8.10 (2H, t), 7.89~8.02 |
| | (7H, m), 7.25~7.73 (22H, m) |
| 1-173 | δ = 8.55 (1H, d), 8.10~8.12 (2H, t), 8.02 (1H, |
| | s), 7.89~7.94 (3H, m), 7.79 (1H, d), 7.25~7.66 |
| | (26H, m) |
| 1-174 | δ = 8.55 (1H, d), 8.10~8.12 (2H, t), 8.02 (1H, |
| | s), 7.25~7.89 (30H, m) |
| 1-179 | δ = 8.55 (1H, d), 8.10~8.12 (2H, t), 7.90~7.94 |
| | (2H, m), 7.79 (1H, d), 7.25~7.63 (13H, m) |
| 2-18 | δ = 8.52 (1H, d), 8.28~8.24 (3H, m), 8.12 (1H, |
| | d), 7.94~7.89 (2H, d), 7.75~7.70 (2H, m), |
| | 7.62~7.25 (19H, m) |
| 2-19 | δ = 8.53 (1H, d), 8.28~8.18 (4H, m), 7.94~7.89 |
| | (2H, q), 7.79~7.70 (3H, m), 7.62~7.25 (22H, m) |
| 2-131 | δ = 8.55 (1H, d), 8.28 (4H, d), 7.94~7.87 (3H, |
| | m), 7.77~7.73 (5H, m), 7.52~7.25 (15H, m) |
| 2-133 | δ = 8.28 (4H, d), 8.18 (1H, d), 8.0 (1H, d), |
| | 7.89~7.87 (2H, t), 7.77~7.73 (5H, m), 7.62 (1H, |
| | m), 7.52~7.41 (18H, m) |
| 2-135 | δ = 8.55 (1H, d), 8.28 (4H, m), 8.12 (1H, d), |
| | 7.94~7.89 (2H, m), 7.75~7.73 (2H, d), 7.63~7.25 |
| | (21H, m) |
| 2-138 | δ = 8.39 (1H, d), 8.28 (4H, d), 8.12~8.09 (2H, t), |
| | 7.89 (1H, d), 7.75~7.61 (6H, m), 7.51~7.41(10H, m) |
| 2-147 | δ = 8.55 (1H, d), 8.28~8.24 (3H, m), 7.94~7.87 |
| | (3H, m), 7.73~7.25 (25H, m) |
| 2-150 | δ = 8.55 (1H, d), 8.28 (2H, d), 8.12 (1H, d), |
| | 7.94~7.85 (4H, m), 7.75~7.73 (2H, d), 7.63~7.62 |
| | (2H, d), 7.52~7.25 (16H, m) |
| 2-264 | δ = 8.55 (1H, d), 8.28 (2H, d), 8.12 (1H, d), |
| | 7.94~7.25 (22H, m) |
| 2-265 | δ = 8.28 (2H, m), 8.18~8.12 (2H, m), 8.00 (1H, |
| | d), 7.89~7.73 (7H, m), 7.66~7.62 (3H, m), |
| | 7.51~7.29 (15H, m) |
| 2-271 | δ = 8.55 (2H, d), 8.36(4H, t), 7.98~7.94(3H, m), |
| | 7.82(1H, d), 7.69~7.50(15H, m), 7.35(2H, t), |
| | 7.26~7.25(2H, d), 7.16(2H, t) |
| 2-285 | δ = 8.55 (1H, d), 8.28 (2H, d), 8.18 (1H, d), |
| | 7.94~7.73 (7H, m), 7.62 (2H, d), 7.52~7.25 (19H, m) |
| 2-286 | δ = 8.55 (1H, d), 8.28 (2H, d), 7.94~7.85 (5H, |
| | m), 7.77~7.69 (3H, m), 7.62 (1H, d), 7.51~7.25 |
| | (20H, m) |
| 2-287 | δ = 8.36(4H, m), 8.03(1H, d), 7.88~7.76(7H, m), |
| | 7.50~7.41(9H, m) |
| 2-289 | δ = 8.36(4H, m), 8.09~7.99(4H, m), 7.88~7.76(5H, |
| | m), 7.63~7.50(9H, m), 7.38(1H, d) |
| 2-291 | δ = 9.27(1H, s), 8.79(1H, d), 8.37~8.30(8H, m), |
| | 8.03(1H, d), 7.88~7.64(9H, m), 7.52~7.50(7H, m) |
| 2-292 | δ = 8.45(1H, d), 8.36(4H, m), 8.12(2H, m), |
| | 8.03~7.76(8H, m), 7.56~7.49(8H, m) |
| 2-295 | δ = 8.36(4H, m), 8.03~7.98(2H, m), 7.88~7.76(8H, |
| | m), [symbol] 7.96~7.79(8H, m), 7.69(1H, d), |
| | 7.60~7.41(12H, m) |
| 2-300 | δ = 8.95(1H, d), 8.50(1H, d), 8.36(4H, m), |
| | 8.20(1H, m), 8.09(1H, m), 7.88~7.69(8H, m), |
| | 7.61~7.50(10H, d), 7.39(1H, t) |
| 2-305 | δ = 9.60(1H, d), 9.27(1H, s), 8.37~8.30(7H, m), |
| | 7.88~7.50(22H, m) |
| 2-311 | δ = 8.36(4H, m), 8.03(1H, d), 7.83~7.79(7H, m), |
| | 7.50~7.41(9H, d), 7.25(4H, d) |
| 2-314 | δ = 9.27(1H, s), 8.79(1H, d), 8.37~8.30(8H, m), |
| | 7.94~7.50(25H, m) |
| 2-315 | δ = 9.27(1H, d), 8.79(1H, d), 8.37~8.30(8H, m), |
| | 8.03(1H, d), 7.94~7.61(13H, m), 7.52~7.50(7H, m) |
| 2-316 | δ = 9.27(1H, s), 8.79(1H, d), 8.37~8.30(8H, m), |
| | 8.03(1H, d), 7.94~7.50(24H, m) |
| 2-319 | δ = 8.55(1H, d), 8.36(4H, m), 8.21(1H, s), |
| | 8.03~7.68(13H, m), 7.60(1H, m), 7.50~7.35(11H, |
| | m), 7.16(1H, t) |
| 2-327 | δ = 9.27(1H, s), 8.79(1H, d), 8.37~8.30(6H, m), |
| | 8.08(1H, d), 7.98~7.50(22H, m), 7.39~7.31(2H, m) |
| 2-333 | δ = 9.27(1H, s), 8.79(1H, d), 8.37~8.30(6H, m), |
| | 7.98~7.50(23H, m), 7.39~7.31(2H, m) |
| 2-337 | δ = 9.27(1H, s), 8.79(1H, d), 8.55(1H, d), |
| | 8.45~8.30(7H, m), 7.98~7.50(23H, m) |
| 2-341 | δ = 9.27(1H, s), 8.79(1H, d), 8.37~8.30(6H, d), |
| | 8.03(1H, d), 7.94~7.47(23H, m), 7.28(1H, t), |
| | 1.69(6H, s) |
| 2-346 | δ = 9.27(1H, s), 8.79(1H, d), 8.37~8.30(6H, m), |
| | 8.03~7.41(29H, m), 7.25(1H, s) |
| 2-352 | δ = 9.27(1H, s), 8.79(1H, d), 8.37~8.30(8H, m), |
| | 8.03~7.96(3H, m), 7.88~7.64(9H, m), 7.52~7.50(7H, |
| | m), 7.25(2H, d) |

### <Experimental Example 1> Manufacture of Organic Light Emitting Device

A glass substrate thinly coated with ITO to have a thickness of 1,500 Å was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transporting layer N, N′-di(1-naphthyl)-N, N′-diphenyl-(1,1′-biphenyl)-4,4′-diamine (NPB) were formed on the ITO transparent electrode (positive electrode).

A light emitting layer was thermally vacuum deposited thereon as follows. The light emitting layer was deposited to have a thickness of 400 Å by using only one type of a compound represented by Chemical Formula 1 or a compound represented by Chemical Formula 2 of the present application as a host or supplying one type compound represented by Chemical Formula 1 of the present application and one type compound represented by Chemical Formula 2 of the present application from each individual supply source, and was deposited by doping the host with Ir(ppy)₃ as a green phosphorescent dopant in an amount of 7%. Thereafter, BCP as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transport layer to form an electron injection layer, and then aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED device were subjected to vacuum sublimed purification under 10-6 to 10-8 torr for each material, and used for the manufacture of OLED.

### <Experimental Example 2> Manufacture of Organic Light Emitting Device

A glass substrate thinly coated with ITO to have a thickness of 1,500 Å was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

As the common layers, the hole injection layer 4,4′,4″-tris[2-naphthyl(phenyl)amino]triphenylamine (2-TNATA) and the hole transporting layer N, N′-di(1-naphthyl)-N, N′-diphenyl-(1,1′-biphenyl)-4,4′-diamine (NPB) were formed on the ITO transparent electrode (positive electrode).

A light emitting layer was thermally vacuum deposited thereon as follows. The light emitting layer was deposited to have a thickness of 400 Å by pre-mixing one type of compound described in Chemical Formula 1 and one type of compound described in Chemical Formula 2 as hosts, and then was deposited from one supply source by doping the host with Ir(ppy)₃ as a green phosphorescent dopant in an amount of 7%. Thereafter, BCP as a hole blocking layer was deposited to have a thickness of 60 Å, and Alq₃ as an electron transport layer was deposited to have a thickness of 200 Å thereon. Finally, lithium fluoride (LiF) was deposited to have a thickness of 10 Å on the electron transporting layer to form an electron injection layer, and then aluminum (Al) negative electrode was deposited to have a thickness of 1,200 Å on the electron injection layer to form a negative electrode, thereby manufacturing an organic electroluminescence device.

Meanwhile, all the organic compounds required for manufacturing an OLED were subjected to vacuum sublimed purification under 10-6 to 10-8 torr for each material, and used for the manufacture of OLED.

For the organic electroluminescence device manufactured as described above, electroluminescence (EL) characteristics were measured by M7000 manufactured by McScience Inc., and based on the measurement result thereof, T90 was measured by a service life measurement equipment (M6000) manufactured by McScience Inc., when the reference luminance was 6,000 cd/m2.

The driving voltages and light emitting efficiencies of the organic electroluminescence devices according to Experimental Examples 1 and 2 are shown in the following Tables 18 to 21.

For reference, the following Tables 18 and 21 are the case where only one type of compound represented by Chemical Formula 1 of the present application or the compound represented by Chemical Formula 2 of the present application is used in Experimental Example 1, that is the case where a single host material is applied, the following Table 19 is the case where the two host compounds in Experimental Example 1 are simultaneously deposited as individual supply sources, and the following Table 20 corresponds to the case where the two light emitting layer compounds in Experimental Example 2 are pre-mixed, and then deposited as one supply source.

TABLE 18

| | Light emitting | Driving | | Color | Service |
|---|---|---|---|---|---|
| | layer | voltage | Efficiency | coordinate | life |
| | Compound | (V) | (cd/A) | (x, y) | (T₉₀) |
| Example | 1-1 | 2.52 | 63.3 | (0.273, | 61 |
| 1 | | | | 0.686) | |
| Example | 1-2 | 2.61 | 59.9 | (0.273, | 62 |
| 2 | | | | 0.691) | |
| Example | 1-4 | 2.57 | 60.4 | (0.265, | 63 |
| 3 | | | | 0.663) | |
| Example | 1-7 | 2.60 | 60.9 | (0.278, | 54 |
| 4 | | | | 0.682) | |
| Example | 1-8 | 2.51 | 61.2 | (0.274, | 59 |
| 5 | | | | 0.677) | |
| Example | 1-12 | 2.51 | 58.2 | (0.269, | 63 |
| 6 | | | | 0.669) | |
| Example | 1-13 | 2.56 | 59.7 | (0.278, | 64 |
| 7 | | | | 0.683) | |
| Example | 1-17 | 2.59 | 58.3 | (0.273, | 56 |
| 8 | | | | 0.686) | |
| Example | 1-18 | 2.58 | 59.2 | (0.273, | 59 |
| 9 | | | | 0.691) | |
| Example | 1-25 | 2.61 | 61.3 | (0.265, | 63 |
| 10 | | | | 0.663) | |
| Example | 1-27 | 2.61 | 62.0 | (0.273, | 66 |
| 11 | | | | 0.688) | |
| Example | 1-30 | 2.52 | 61.1 | (0.276, | 65 |
| 12 | | | | 0.671) | |
| Example | 1-32 | 2.63 | 63.7 | (0.281, | 64 |
| 13 | | | | 0.690) | |
| Example | 1-34 | 2.60 | 58.2 | (0.278, | 61 |
| 14 | | | | 0.683) | |
| Example | 1-37 | 2.62 | 59.7 | (0.273, | 59 |
| 15 | | | | 0.686) | |
| Example | 1-38 | 2.59 | 59.9 | (0.273, | 61 |
| 16 | | | | 0.691) | |
| Example | 1-39 | 2.58 | 60.4 | (0.265, | 63 |
| 17 | | | | 0.663) | |
| Example | 1-40 | 2.61 | 60.9 | (0.278, | 54 |
| 18 | | | | 0.682) | |
| Example | 1-41 | 2.51 | 58.3 | (0.273, | 64 |
| 19 | | | | 0.686) | |
| Example | 1-49 | 2.56 | 59.5 | (0.273, | 64 |
| 20 | | | | 0.691) | |
| Example | 1-50 | 2.63 | 61.3 | (0.265, | 55 |
| 21 | | | | 0.663) | |
| Example | 1-51 | 2.60 | 59.2 | (0.273, | 58 |
| 22 | | | | 0.688) | |
| Example | 1-54 | 2.61 | 61.7 | (0.274, | 62 |
| 23 | | | | 0.677) | |
| Example | 1-56 | 2.51 | 61.0 | (0.269, | 62 |
| 24 | | | | 0.669) | |
| Example | 1-57 | 2.53 | 61.1 | (0.278, | 65 |
| 25 | | | | 0.683) | |
| Example | 1-59 | 2.51 | 58.2 | (0.273, | 65 |
| 26 | | | | 0.686) | |
| Example | 1-61 | 2.56 | 59.7 | (0.273, | 59 |
| 27 | | | | 0.691) | |
| Example | 1-64 | 2.59 | 58.8 | (0.273, | 62 |
| 28 | | | | 0.691) | |
| Example | 1-66 | 2.58 | 59.2 | (0.265, | 64 |
| 29 | | | | 0.663) | |
| Example | 1-73 | 2.61 | 63.7 | (0.278, | 55 |
| 30 | | | | 0.682) | |
| Example | 1-74 | 2.56 | 58.2 | (0.278, | 62 |
| 31 | | | | 0.683) | |
| Example | 1-78 | 2.63 | 59.6 | (0.273, | 66 |
| 32 | | | | 0.686) | |
| Example | 1-80 | 2.60 | 59.9 | (0.273, | 65 |
| 33 | | | | 0.691) | |
| Example | 1-81 | 2.58 | 63.8 | (0.265, | 65 |
| 34 | | | | 0.663) | |
| Example | 1-83 | 2.59 | 61.8 | (0.278, | 55 |
| 35 | | | | 0.682) | |
| Example | 1-85 | 2.57 | 611 | (0.273, | 59 |
| 36 | | | | 0.688) | |
| Example | 1-88 | 2.61 | 61.1 | (0.276, | 61 |
| 37 | | | | 0.671) | |
| Example | 1-90 | 2.64 | 58.3 | (0.281, | 58 |
| 38 | | | | 0.690) | |
| Example | 1-97 | 2.52 | 59.7 | (0.278, | 62 |
| 39 | | | | 0.683) | |
| Example | 1-98 | 2.63 | 59.0 | (0.273, | 66 |
| 40 | | | | 0.686) | |
| Example | 1-102 | 2.59 | 60.3 | (0.273, | 61 |
| 41 | | | | 0.691) | |
| Example | 1-107 | 2.59 | 60.9 | (0.269, | 58 |
| 42 | | | | 0.669) | |
| Example | 1-109 | 2.64 | 58.8 | (0.278, | 54 |
| 43 | | | | 0.683) | |
| Example | 1-111 | 2.51 | 58.4 | (0.273, | 64 |
| 44 | | | | 0.686) | |
| Example | 1-112 | 2.56 | 59.3 | (0.273, | 64 |
| 45 | | | | 0.691) | |
| Example | 1-113 | 2.63 | 59.7 | (0.273, | 55 |
| 46 | | | | 0.686) | |
| Example | 1-114 | 2.60 | 58.9 | (0.273, | 58 |
| 47 | | | | 0.691) | |
| Example | 1-122 | 2.61 | 59.2 | (0.273, | 63 |
| 48 | | | | 0.691) | |
| Example | 1-123 | 2.51 | 63.1 | (0.265, | 64 |
| 49 | | | | 0.663) | |
| Example | 1-124 | 2.54 | 59.9 | (0.278, | 56 |
| 50 | | | | 0.682) | |
| Example | 1-125 | 2.63 | 60.5 | (0.278, | 61 |
| 51 | | | | 0.683) | |
| Example | 1-127 | 2.59 | 60.9 | (0.274, | 62 |
| 52 | | | | 0.677) | |
| Example | 1-128 | 2.59 | 59.9 | (0.269, | 64 |
| 53 | | | | 0.669) | |
| Example | 1-129 | 2.58 | 59.9 | (0.278, | 54 |
| 54 | | | | 0.683) | |
| Example | 1-133 | 2.61 | 60.3 | (0.273, | 59 |
| 55 | | | | 0.686) | |
| Example | 1-148 | 2.56 | 60.9 | (0.273, | 63 |
| 56 | | | | 0.691) | |
| Example | 1-149 | 2.66 | 61.4 | (0.265, | 62 |
| 57 | | | | 0.663) | |
| Example | 1-150 | 2.63 | 59.2 | (0.276, | 66 |
| 58 | | | | 0.671) | |
| Example | 1-151 | 2.61 | 61.6 | (0.281, | 66 |
| 59 | | | | 0.690) | |
| Example | 1-152 | 2.61 | 61.0 | (0.278, | 65 |
| 60 | | | | 0.683) | |
| Example | 1-155 | 2.63 | 61.6 | (0.273, | 63 |
| 61 | | | | 0.686) | |
| Example | 1-156 | 2.51 | 63.7 | (0.273, | 59 |
| 62 | | | | 0.691) | |
| Example | 1-159 | 2.64 | 59.4 | (0.273, | 63 |
| 63 | | | | 0.691) | |
| Example | 1-162 | 2.59 | 60.4 | (0.273, | 65 |
| 64 | | | | 0.691) | |
| Example | 1-163 | 2.61 | 58.1 | (0.265, | 65 |
| 65 | | | | 0.663) | |
| Example | 1-167 | 2.54 | 59.7 | (0.278, | 61 |
| 66 | | | | 0.682) | |
| Example | 1-170 | 2.66 | 61.3 | (0.273, | 63 |
| 67 | | | | 0.691) | |
| Example | 1-171 | 2.62 | 59.1 | (0.265, | 58 |
| 68 | | | | 0.663) | |
| Example | 1-173 | 2.61 | 63.7 | (0.278, | 59 |
| 69 | | | | 0.682) | |
| Example | 1-174 | 2.58 | 58.9 | (0.278, | 62 |
| 70 | | | | 0.683) | |
| Example | 1-179 | 2.61 | 59.2 | (0.273, | 64 |
| 71 | | | | 0.686) | |

As can be seen in Table 18, Examples 1 to 71 have bulkier structures by introducing an additional heteroaryl into a basic biscarbazole structure and additionally introducing an aryl substituent into the same phenyl into which biscarbazole has been introduced. Examples 1 to 71 are in a form where the number of substituents to a biscarbazole structure is increased, and have an effect that when the region of the HOMO level is expanded more widely and the HOMO level is widened, the overall charge balance is stabilized in the device by adjusting the hole mobility.

In general, in the case of an aryl group, the stability of the molecular structure may be enhanced by expanding the region of the HOMO level. In addition, since the aryl group has a wide n-conjugation region in an expanded form while the aryl group is introduced, the aryl group can also be used as an auxiliary means for adjusting the energy band gap.

A substituent in the form of a heteroaryl such as dibenzofuran has a wide I-conjugation region in a form similar to an aryl group. Furthermore, since the substituent has a more rigid structure, it is possible to maintain a high T1 energy level, and simultaneously to see an effect of adjusting the energy band gap. However, since the substituent has the most bulky structure, the substituent has an effect that the driving voltage is relatively increased, but has high structural stability, and thus is helpful for having longer service life characteristics.

TABLE 19

| | | | | Driving | | Color | Service |
|---|---|---|---|---|---|---|---|
| | Compound | Compound | | voltage | Efficiency | coordinate | life |
| | 1 | 2 | Ratio | (V) | (cd/A) | (x, y) | (T₉₀) |
| Comparative | 2-17 | A | 1:1 | 5.70 | 76.4 | (0.278, | 185 |
| Example 1 | | | | | | 0.667) | |
| Comparative | | B | | 6.82 | 79.2 | (0.290, | 234 |
| Example 2 | | | | | | 0.685) | |
| Comparative | 2-19 | B | 1:1 | 5.59 | 83.7 | (0.283, | 259 |
| Example 3 | | | | | | 0.672) | |
| Comparative | | C | | 5.37 | 84.8 | (0.283, | 301 |
| Example 4 | | | | | | 0.668) | |
| Comparative | 2-131 | D | 1:1 | 5.66 | 77.1 | (0.277, | 263 |
| Example 5 | | | | | | 0.683) | |
| Comparative | | E | | 5.57 | 84.2 | (0.275, | 258 |
| Example 6 | | | | | | 0.677) | |
| Comparative | 2-18 | B | 1:2 | 5.77 | 73.2 | (0.272, | 249 |
| Example 7 | | | | | | 0.681) | |
| Comparative | | | 1:1 | 5.65 | 81.3 | (0.279, | 267 |
| Example 8 | | | | | | 0.673) | |
| Comparative | 2-131 | C | 1:2 | 6.41 | 62.1 | (0.261, | 278 |
| Example 9 | | | | | | 0.666) | |
| Comparative | | | 1:1 | 6.02 | 66.8 | (0.266, | 269 |
| Example 10 | | | | | | 0.653) | |
| Example 85 | 2-17 | 1-1 | 1:8 | 4.11 | 116.5 | (0.258, | 450 |
| | | | | | | 0.679) | |
| Example 86 | | | 1:5 | 3.62 | 122.5 | (0.277, | 457 |
| | | | | | | 0.680) | |
| Example 87 | | | 1:2 | 3.22 | 125.4 | (0.276, | 468 |
| | | | | | | 0.676) | |
| Example 88 | | | 1:1 | 3.01 | 127.7 | (0.278, | 471 |
| | | | | | | 0.673) | |
| Example 89 | | | 2:1 | 3.16 | 125.1 | (0.274, | 466 |
| | | | | | | 0.675) | |
| Example 90 | | | 5:1 | 3.32 | 124.3 | (0.276, | 455 |
| | | | | | | 0.670) | |
| Example 91 | | | 8:1 | 4.24 | 118.6 | (0.274, | 450 |
| | | | | | | 0.679) | |
| Example 92 | 2-19 | 1-2 | 1:2 | 3.19 | 116.3 | (0.277, | 463 |
| | | | | | | 0.677) | |
| Example 93 | | | 1:1 | 3.18 | 119.6 | (0.276, | 474 |
| | | | | | | 0.678) | |
| Example 94 | 2-131 | 1-13 | 1:2 | 3.28 | 117.2 | (0.277, | 462 |
| | | | | | | 0.672) | |
| Example 95 | | | 1:1 | 3.21 | 119.4 | (0.273, | 482 |
| | | | | | | 0.681) | |
| Example 96 | 2-18 | 1-97 | 1:2 | 3.26 | 119.1 | (0.286, | 470 |
| | | | | | | 0.674) | |
| Example 97 | | | 1:1 | 3.19 | 122.0 | (0.282, | 478 |
| | | | | | | 0.672) | |
| Example 98 | 2-133 | 1-98 | 1:2 | 3.11 | 120.7 | (0.271, | 472 |
| | | | | | | 0.677) | |
| Example 99 | | | 1:1 | 3.28 | 119.5 | (0.268, | 466 |
| | | | | | | 0.681) | |
| Example 100 | 2-135 | 1-107 | 1:2 | 3.33 | 121.6 | (0.274, | 505 |
| | | | | | | 0.671) | |
| Example 101 | | | 1:1 | 3.25 | 124.2 | (0.279, | 479 |
| | | | | | | 0.673) | |
| Example 102 | 2-138 | 1-111 | 1:2 | 3.32 | 120.4 | (0.274, | 492 |
| | | | | | | 0.683) | |
| Example 103 | | | 1:1 | 3.29 | 123.1 | (0.272, | 501 |
| | | | | | | 0.673) | |
| Example 104 | 2-147 | 1-127 | 1:2 | 3.34 | 126.5 | (0.281, | 510 |
| | | | | | | 0.6750 | |
| Example 105 | | | 1:1 | 3.32 | 129.6 | (0.278, | 514 |
| | | | | | | 0.678) | |
| Example 106 | 2-150 | 1-128 | 1:2 | 3.40 | 127.4 | (0.280, | 509 |
| | | | | | | 0.680) | |
| Example 107 | | | 1:1 | 3.38 | 131.5 | (0.281, | 511 |
| | | | | | | 0.681) | |
| Example 108 | 2-264 | 1-133 | 1:2 | 3.54 | 133.5 | (0.274, | 519 |
| | | | | | | 0.678) | |
| Example 109 | | | 1:1 | 3.51 | 135.2 | (0.275, | 525 |
| | | | | | | 0.677) | |
| Example 110 | 2-265 | 1-156 | 1:2 | 3.53 | 133.1 | (0.272, | 540 |
| | | | | | | 0.681) | |
| Example 111 | | | 1:1 | 3.49 | 137.6 | (0.277, | 548 |
| | | | | | | 0.681) | |
| Example 112 | 2-271 | 1-163 | 1:2 | 3.62 | 133.1 | (0.275, | 520 |
| | | | | | | 0.682) | |
| Example 113 | | | 1:1 | 3.55 | 136.4 | (0.281, | 529 |
| | | | | | | 0.680) | |
| Example 114 | 2-285 | 1-173 | 1:3 | 3.51 | 124.7 | (0.281, | 501 |
| | | | | | | 0.679) | |
| Example 115 | 2-285 | 1-173 | 1:2 | 3.46 | 127.4 | (0.280, | 510 |
| | | | | | | 0.678) | |
| Example 116 | | | 1:1 | 3.44 | 130.5 | (0.289, | 518 |
| | | | | | | 0.683) | |
| Example 117 | | 1-174 | 1:2 | 3.45 | 134.2 | (0.286, | 516 |
| | | | | | | 0.678) | |
| Example 118 | | | 1:1 | 3.41 | 139.8 | (0.280, | 525 |
| | | | | | | 0.679) | |
| Example 119 | | 1-179 | 1:2 | 3.54 | 134.6 | (0.273, | 537 |
| | | | | | | 0.680) | |
| Example 120 | | | 1:1 | 3.49 | 140.1 | (0.283, | 542 |
| | | | | | | 0.681) | |

TABLE 20

| | | | | Driving | | Color | Service |
|---|---|---|---|---|---|---|---|
| | Compound | Compound | | voltage | Efficiency | coordinate | life |
| | 1 | 2 | Ratio | (V) | (cd/A) | (x, y) | (T₉₀) |
| Comparative | 2-17 | A | 1:1 | 5.90 | 85.7 | (0.281, | 264 |
| Example 11 | | | | | | 0.679) | |
| Comparative | | B | 1:1 | 5.56 | 90.5 | (0.284, | 306 |
| Example 12 | | | | | | 0.672) | |
| Comparative | 2-131 | D | 1:1 | 5.66 | 911.7 | (0.273, | 321 |
| Example 13 | | | | | | 0.683) | |
| Comparative | | E | 1:1 | 6.23 | 75.6 | (0.281, | 295 |
| Example 14 | | | | | | 0.683) | |
| Comparative | 2-18 | B | 1:1 | 5.89 | 87.4 | (0.268, | 344 |
| Example 15 | | | | | | 0.682) | |
| Comparative | 2-19 | B | 1:1 | 6.02 | 71.4 | (0.278, | 257 |
| Example 16 | | | | | | 0.673) | |
| Comparative | 2-264 | C | 1:1 | 5.68 | 85.2 | (0.286, | 331 |
| Example 17 | | | | | | 0.669) | |
| Example 121 | 2-17 | 1-1 | 1:1 | 3.09 | 125.5 | (0.277, | 581 |
| | | | | | | 0.673) | |
| Example 122 | | 1-2 | 1:1 | 3.54 | 141.3 | (0.279, | 680 |
| | | | | | | 0.675) | |
| Example 123 | 2-131 | 1-13 | 1:1 | 3.19 | 131.8 | (0.273, | 546 |
| | | | | | | 0.683) | |
| Example 124 | 2-18 | 1-97 | 1:1 | 3.25 | 134.6 | (0.278, | 615 |
| | | | | | | 0.682) | |
| Example 125 | | 1-98 | 1:1 | 3.62 | 149.4 | (0.281, | 722 |
| | | | | | | 0.681) | |
| Example 126 | 2-19 | 1-107 | 1:1 | 3.14 | 134.8 | (0.275, | 562 |
| | | | | | | 0.670) | |
| Example 127 | | 1-111 | 1:1 | 3.51 | 150.8 | (0.272, | 711 |
| | | | | | | 0.686) | |
| Example 128 | 2-286 | 1-127 | 1:1 | 3.28 | 132.6 | (0.275, | 622 |
| | | | | | | 0.678) | |
| Example 129 | | 1-156 | 1:1 | 3.56 | 148.4 | (0.278, | 726 |
| | | | | | | 0.675) | |
| Example 130 | 2-392 | 1-169 | 1:1 | 3.49 | 132.2 | (0.272, | 648 |
| | | | | | | 0.681) | |
| Example 131 | | 1-178 | 1:1 | 3.48 | 132.7 | (0.277, | 721 |
| | | | | | | 0.680) | |
| Example 132 | 2-412 | 1-1 | 1:1 | 3.40 | 128.0 | (0.278, | 645 |
| | | | | | | 0.673) | |
| Example 133 | | 1-23 | 1:1 | 3.38 | 128.7 | (0.274, | 719 |
| | | | | | | 0.675) | |
| Example 134 | 2-432 | 1-97 | 1:1 | 3.37 | 133.5 | (0.280, | 627 |
| | | | | | | 0.680) | |
| Example 135 | | 1-115 | 1:1 | 3.37 | 135.2 | (0.281, | 725 |
| | | | | | | 0.681) | |

Compounds A to E used in Tables 19 and 20 are as follows.

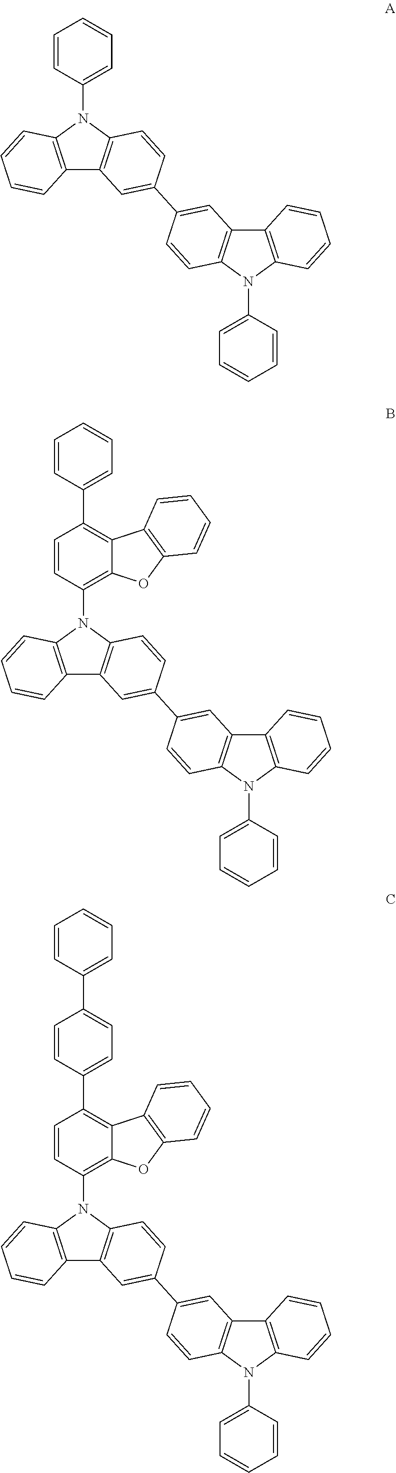 [SMILES: c1ccc(-n2c3ccccc3c3cc(-c4ccc5c(c4)c4ccccc4n5-c4ccccc4)ccc32)cc1 ; c1ccc(-c2ccc(-n3c4ccccc4c4cc(-c5ccc6c(c5)c5ccccc5n6-c5ccccc5)ccc43)c3oc4ccccc4c23)cc1 ; c1ccc(-c2ccc(-c3ccc(-n4c5ccccc5c5cc(-c6ccc7c(c6)c6ccccc6n7-c6ccccc6)ccc54)c4oc5ccccc5c34)cc2)cc1]

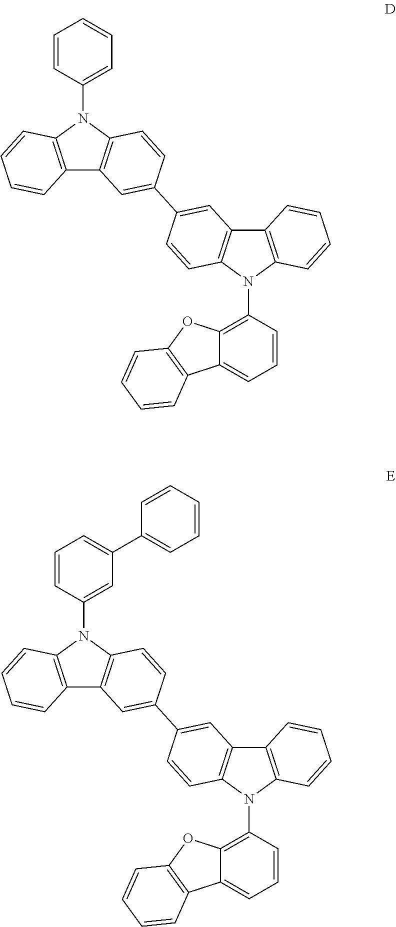 [SMILES: c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1]

TABLE 21

| | | | | | |
|---|---|---|---|---|---|
| Comparative | 2-17 | 3.92 | 78.2 | (0.276, | 147 |
| Example 18 | | | | 0.672) | |
| Comparative | 2-18 | 3.75 | 71.3 | (0.278, | 173 |
| Example 19 | | | | 0.673) | |
| Comparative | 2-131 | 3.81 | 62.9 | (0.283, | 155 |
| Example 20 | | | | 0.673) | |
| Comparative | 2-133 | 3.99 | 70.6 | (0.274, | 169 |
| Example 21 | | | | 0.679) | |
| Comparative | 2-135 | 3.82 | 72.4 | (0.272, | 168 |
| Example 22 | | | | 0.676) | |
| Comparative | 2-138 | 3.72 | 73.6 | (0.273, | 174 |
| Example 23 | | | | 0.675) | |
| Comparative | 2-147 | 4.43 | 63.4 | (0.283, | 132 |
| Example 24 | | | | 0.677) | |
| Comparative | 2-150 | 3.89 | 75.9 | (0.284, | 142 |
| Example 25 | | | | 0.676) | |
| Comparative | 2-264 | 3.94 | 70.2 | (0.273, | 154 |
| Example 26 | | | | 0.678) | |
| Comparative | 2-265 | 3.99 | 74.2 | (0.274, | 166 |
| Example 27 | | | | 0.672) | |
| Comparative | 2-271 | 4.02 | 72.3 | (0.275, | 146 |
| Example 28 | | | | 0.680) | |
| Comparative | 2-285 | 3.71 | 80.4 | (0.274, | 180 |
| Example 29 | | | | 0.677) | |
| Comparative | 2-286 | 3.81 | 79.3 | (0.275, | 178 |
| Example 30 | | | | 0.678) | |

Further, as can be seen in Tables 18 to 21, rather than the case where the compound represented by Chemical Formula 1 of the present application or the compound represented by Chemical Formula 2 of the present application is used alone, the case where both the compound of Chemical Formula 1 and the compound of Chemical Formula 2 are comprised in the organic material layer of the organic light emitting device exhibits better efficiency and service life effects. From this result, it can be expected that an exciplex phenomenon will occur when two compounds are simultaneously comprised.

The exciplex phenomenon is a phenomenon in which energy with a magnitude of the HOMO level of the donor (p-host) and the LUMO level of the acceptor (n-host) is released as an electron exchange between two molecules. When the exciplex phenomenon between two molecules occurs, a reverse intersystem crossing (RISC) occurs, and the internal quantum efficiency of fluorescence can be increased to 100% due to the RISC. When a donor with a good hole transport capacity (p-host) and an acceptor with a good electron transport capacity (n-host) are used as hosts for the light emitting layer, holes are injected into the p-host and electrons are injected into the n-host, so that the driving voltage can be lowered, which can help to improve the service life.

In addition, as can be seen from Tables 19 and 20, it could be confirmed that when a light emitting host composed of a plurality of compounds was deposited by pre-mixing the compounds, and then forming the host by one deposition supply source, the efficiency and service life of the device were improved, compared to when a plurality of compounds is supplied by individual supply sources, and then deposited. This means that since the deposition is not conducted several times, the uniformity and thin film characteristics of the thin film may be improved, the process procedures may be simplified, the cost may be reduced, and a device in which the efficiency and service life have been improved may be formed.

Furthermore, referring to Comparative Examples 1 to 17, it could be confirmed that even though the compound was not used alone, the performance of the device is excellent when the compound represented by Chemical Formula 1 of the present application and the compound represented by Chemical Formula 2 of the present application were simultaneously used.

Further, referring to Comparative Examples 18 to 30, it could be confirmed that the case where the compound represented by Chemical Formula 1 of the present application and the compound represented by Chemical Formula 2 of the present application were used together had a low driving voltage while having a service life appropriate for use as a device compared to the case where the compound represented by Chemical Formula 2 of the present application is used alone.

Specifically, when Comparative Example 16 and Example 126 are compared in Table 20, the case where Compound 2 is B has a structure in which biscabazole is bonded to dibenzofuran at position 4, and the aryl group is bonded to position 3. Compound 1-107 of the present invention exhibits a characteristic in that stacking is uniformly performed and thus the service life is increased because a phenyl group is introduced into dibenzofuran at position 3 to form a rigid structure.

In addition, when Comparative Example 11 and Example 121 are compared, it can be confirmed that the efficiency and service life are improved because Compound 1-1 of the present invention has a molecular weight and structural stability appropriate for use as a P-host.

When Comparative Example 13 and Example 123 are compared, the case where Compound 2 used for the device is Compound D is in a form where an aryl group is not introduced into the end of dibenzofuran, and the driving voltage is increased and the efficiency is reduced because the region of the HOMO level is not expanded more widely, and thus the hole mobility becomes fast and the charge balance is affected.

In contrast, it could be confirmed that the compound of the present invention can appropriately adjust the hole mobility by introducing an aryl group into dibenzofuran and stabilizes the charge balance, and the driving voltage and efficiency are improved.

<Explanation of Reference Numerals and Symbols>

| | |
|---|---|
| 100: | Substrate |
| 200: | Positive electrode |
| 300: | Organic material layer |
| 301: | Hole injection layer |
| 302: | Hole transport layer |
| 303: | Light emitting layer |
| 304: | Hole blocking layer |
| 305: | Electron transport layer |
| 306: | Electron injection layer |
| 400: | Negative electrode |

1. A heterocyclic compound represented by the following Chemical Formula 1: 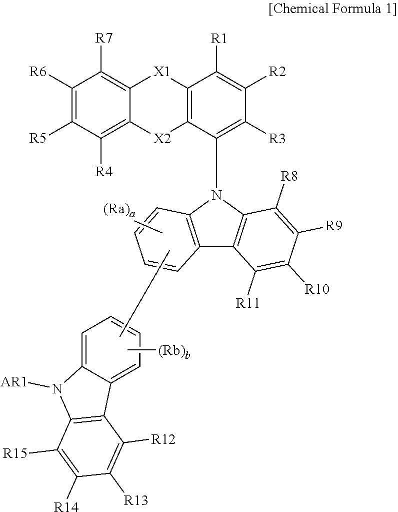
in Chemical Formula 1,
one of X1 and X2 is a direct bond, and the other is O,
R1 to R15, Ra and Rb are the same as or different from each other, and are each independently hydrogen; deuterium; a substituted or unsubstituted alkyl group having 1 to 60 carbon atoms; a substituted or unsubstituted aryl group having 6 to 60 carbon atoms; a substituted or unsubstituted heteroaryl group having 2 to 60 carbon atoms; or —NRcRd, and Rc and Rd are the same as or different from each other, and are each independently a substituted or unsubstituted aryl group having 6 to 60 carbon atoms; or a substituted or unsubstituted heteroaryl group having 2 to 60 carbon atoms,
at least one of R1 to R3 is a substituted or unsubstituted aryl group having 6 to 60 carbon atoms,
AR1 is a substituted or unsubstituted aryl group having 6 to 60 carbon atoms,
when X2 is O, R1 is hydrogen; or deuterium, and
a and b are an integer from 0 to 3, and when a is 2 or higher, Ra's in the parenthesis are the same as or different from each other, and when b is 2 or higher, Rb's in the parenthesis are the same as or different from each other.

2. The heterocyclic compound of claim 1, wherein Chemical Formula 1 is represented by the following Chemical Formula 1-1 or 1-2: 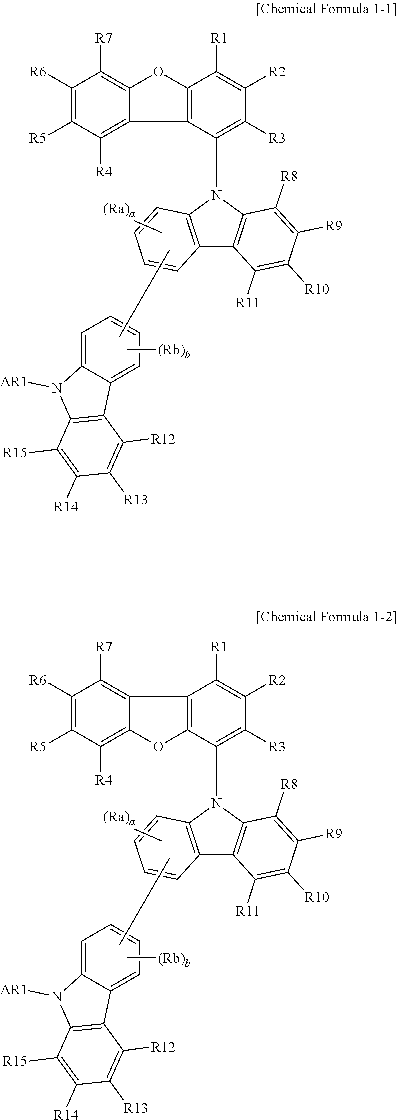
in Formulae 1-1 and 1-2,
R31 is hydrogen; or deuterium,
R21 to R23, R32 and R33 are the same as or different from each other, and are each independently hydrogen; deuterium; a substituted or unsubstituted alkyl group having 1 to 60 carbon atoms; a substituted or unsubstituted aryl group having 6 to 60 carbon atoms; a substituted or unsubstituted heteroaryl group having 2 to 60 carbon atoms; or —NRcRd, and Rc and Rd are the same as or different from each other, and are each independently a substituted or unsubstituted aryl group having 6 to 60 carbon atoms; or a substituted or unsubstituted heteroaryl group having 2 to 60 carbon atoms,
at least one of R21 to R23 is a substituted or unsubstituted aryl group having 6 to 60 carbon atoms,
at least one of R32 and R33 is a substituted or unsubstituted aryl group having 6 to 60 carbon atoms, and
the definitions of R4 to R15, Ra, Rb, AR1, a and b are the same as those in Chemical Formula 1.

3. The heterocyclic compound of claim 1, wherein when R1 to R15, Ra, Rb and AR1 have a substituent substituted with deuterium, the contents of substituted deuterium of R1 to R15, Ra, Rb and AR1 are each independently 0% to 100%.

4. The heterocyclic compound of claim 1, wherein R4 to R15, Ra and Rb are the same as or different from each other, and are each independently hydrogen; deuterium; or a substituted or unsubstituted aryl group having 6 to 40 carbon atoms.

5. The heterocyclic compound of claim 1, wherein AR1 is a phenyl group which is unsubstituted or substituted with one or more deuteriums; or a biphenyl group which is unsubstituted or substituted with one or more deuteriums.

6. The heterocyclic compound of claim 1, wherein R1 to R3 are the same as or different from each other, and are each independently hydrogen; deuterium; or a substituted or unsubstituted aryl group having 6 to 40 carbon atoms, R4 to R15, Ra and Rb are the same as or different from each other, and are each independently hydrogen; or deuterium, and at least one of R1 to R3 is a substituted or unsubstituted aryl group having 6 to 40 carbon atoms.

7. The heterocyclic compound of claim 1, wherein Chemical Formula 1 is represented by any one of the following compounds:

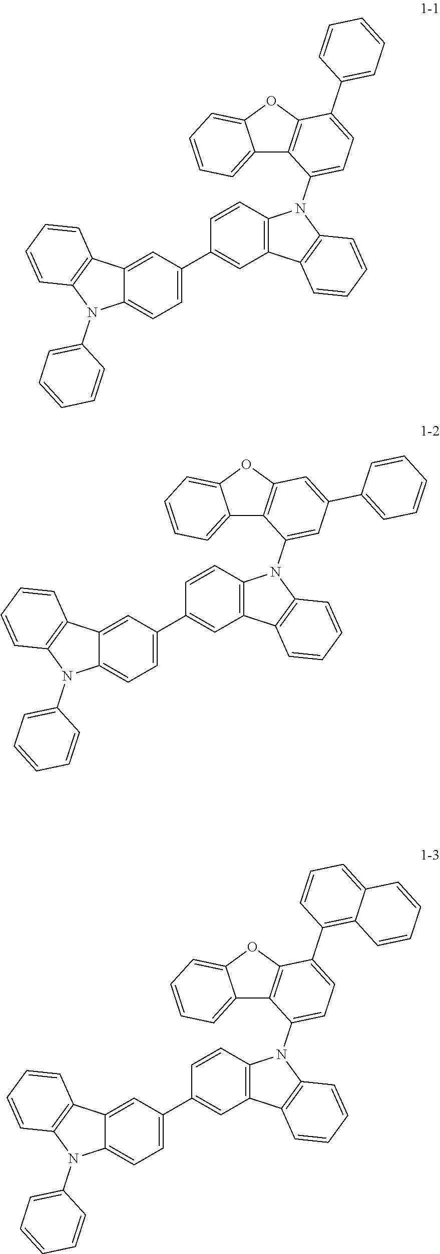 [SMILES: c1ccc(-c2ccc(-n3c4ccccc4c4cc(-c5ccc6c(c5)c5ccccc5n6-c5ccccc5)ccc43)c3c2oc2ccccc23)cc1 ; c1ccc(-c2cc(-n3c4ccccc4c4cc(-c5ccc6c(c5)c5ccccc5n6-c5ccccc5)ccc43)c3c(c2)oc2ccccc23)cc1 ; c1ccc(-n2c3ccccc3c3cc(-c4ccc5c(c4)c4ccccc4n5-c4ccc(-c5cccc6ccccc56)c5oc6ccccc6c45)ccc32)cc1]

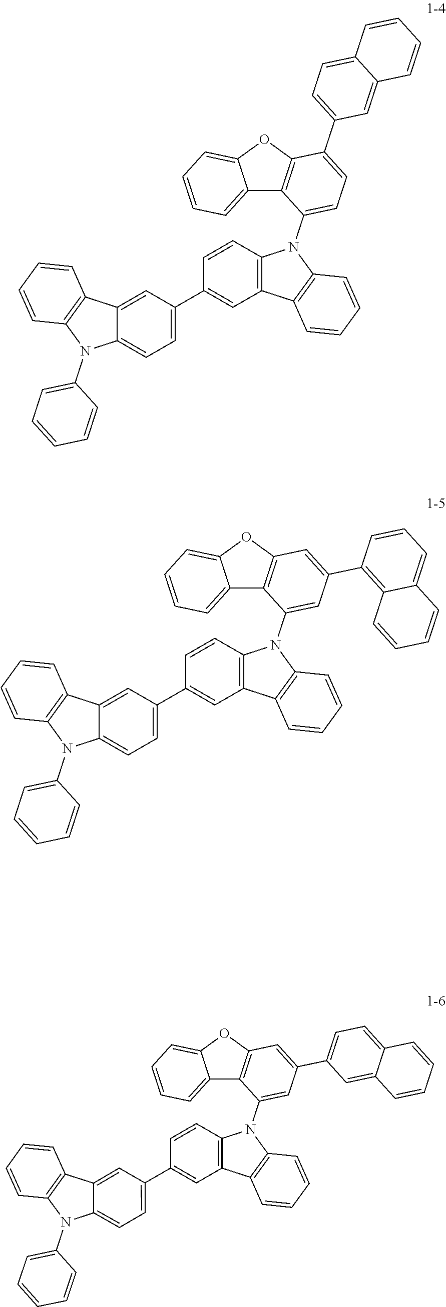 [SMILES: c1ccc(-n2c3ccccc3c3cc(-c4ccc5c(c4)c4ccccc4n5-c4ccc(-c5ccc6ccccc6c5)c5oc6ccccc6c45)ccc32)cc1 ; c1ccc(-n2c3ccccc3c3cc(-c4ccc5c(c4)c4ccccc4n5-c4cc(-c5cccc6ccccc56)cc5oc6ccccc6c45)ccc32)cc1 ; c1ccc(-n2c3ccccc3c3cc(-c4ccc5c(c4)c4ccccc4n5-c4cc(-c5ccc6ccccc6c5)cc5oc6ccccc6c45)ccc32)cc1]

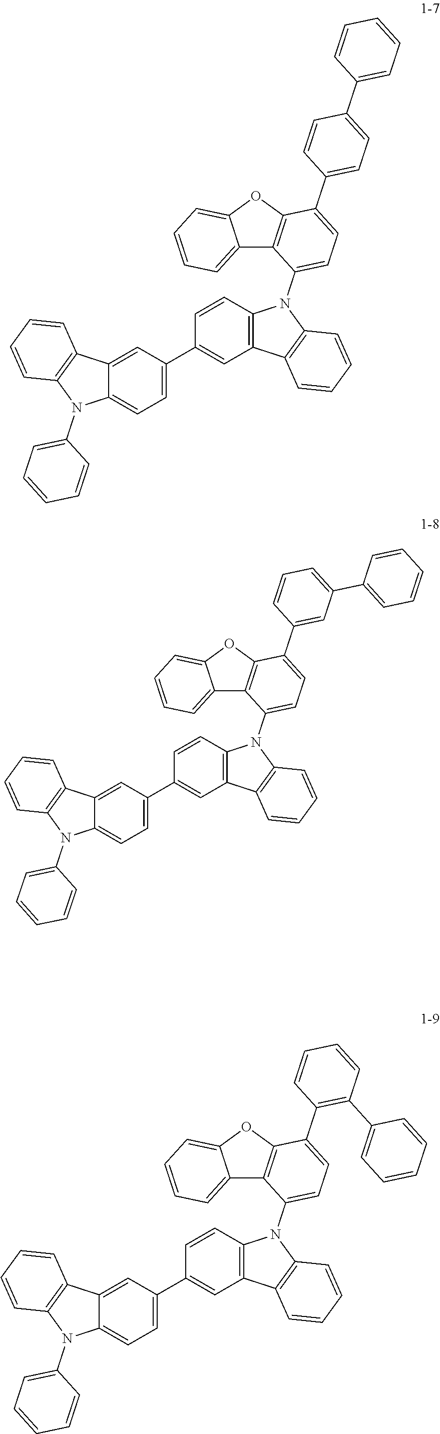 [SMILES: c1ccc(-c2ccc(-c3ccc(-n4c5ccccc5c5cc(-c6ccc7c(c6)c6ccccc6n7-c6ccccc6)ccc54)c4c3oc3ccccc34)cc2)cc1 ; c1ccc(-c2cccc(-c3ccc(-n4c5ccccc5c5cc(-c6ccc7c(c6)c6ccccc6n7-c6ccccc6)ccc54)c4c3oc3ccccc34)c2)cc1 ; c1ccc(-c2ccccc2-c2ccc(-n3c4ccccc4c4cc(-c5ccc6c(c5)c5ccccc5n6-c5ccccc5)ccc43)c3c2oc2ccccc23)cc1]

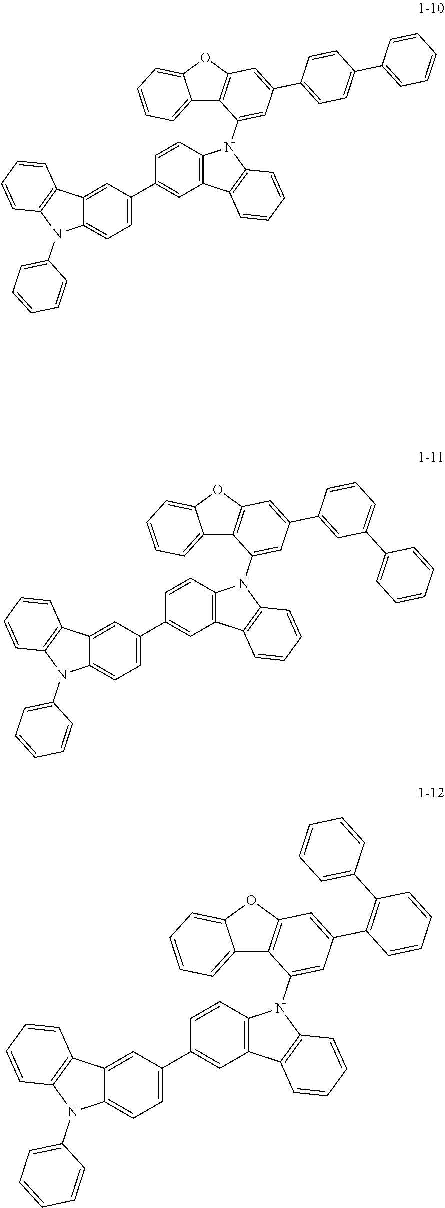 [SMILES: c1ccc(-c2ccc(-c3cc(-n4c5ccccc5c5cc(-c6ccc7c(c6)c6ccccc6n7-c6ccccc6)ccc54)c4c(c3)oc3ccccc34)cc2)cc1 ; c1ccc(-c2cccc(-c3cc(-n4c5ccccc5c5cc(-c6ccc7c(c6)c6ccccc6n7-c6ccccc6)ccc54)c4c(c3)oc3ccccc34)c2)cc1 ; c1ccc(-c2ccccc2-c2cc(-n3c4ccccc4c4cc(-c5ccc6c(c5)c5ccccc5n6-c5ccccc5)ccc43)c3c(c2)oc2ccccc23)cc1]

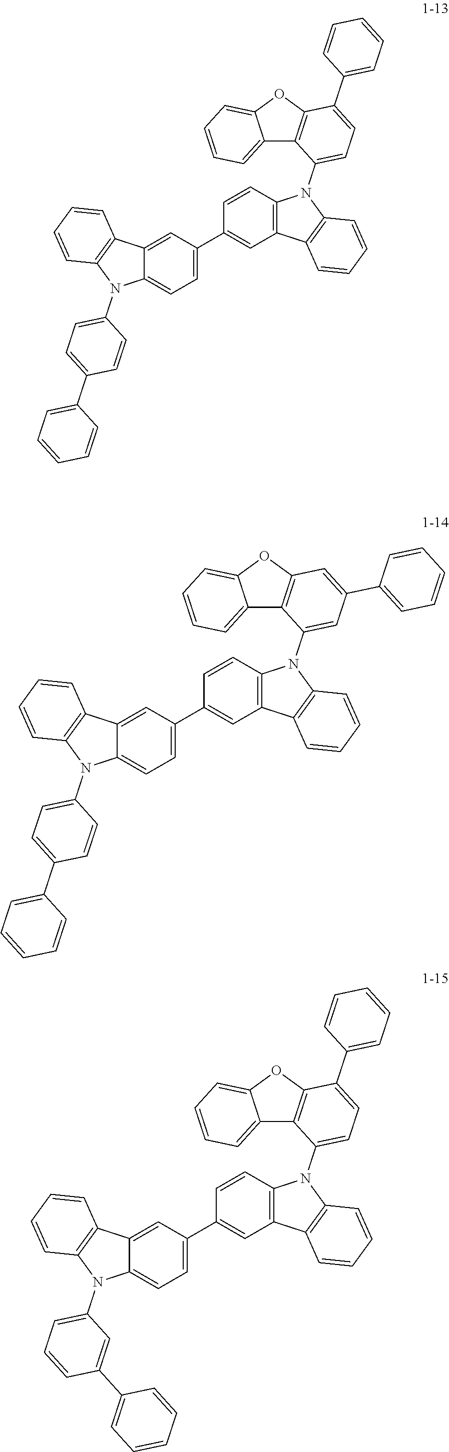 [SMILES: c1ccc(-c2ccc(-n3c4ccccc4c4cc(-c5ccc6c(c5)c5ccccc5n6-c5ccc(-c6ccccc6)c6oc7ccccc7c56)ccc43)cc2)cc1 ; c1ccc(-c2ccc(-n3c4ccccc4c4cc(-c5ccc6c(c5)c5ccccc5n6-c5cc(-c6ccccc6)cc6oc7ccccc7c56)ccc43)cc2)cc1 ; c1ccc(-c2cccc(-n3c4ccccc4c4cc(-c5ccc6c(c5)c5ccccc5n6-c5ccc(-c6ccccc6)c6oc7ccccc7c56)ccc43)c2)cc1]

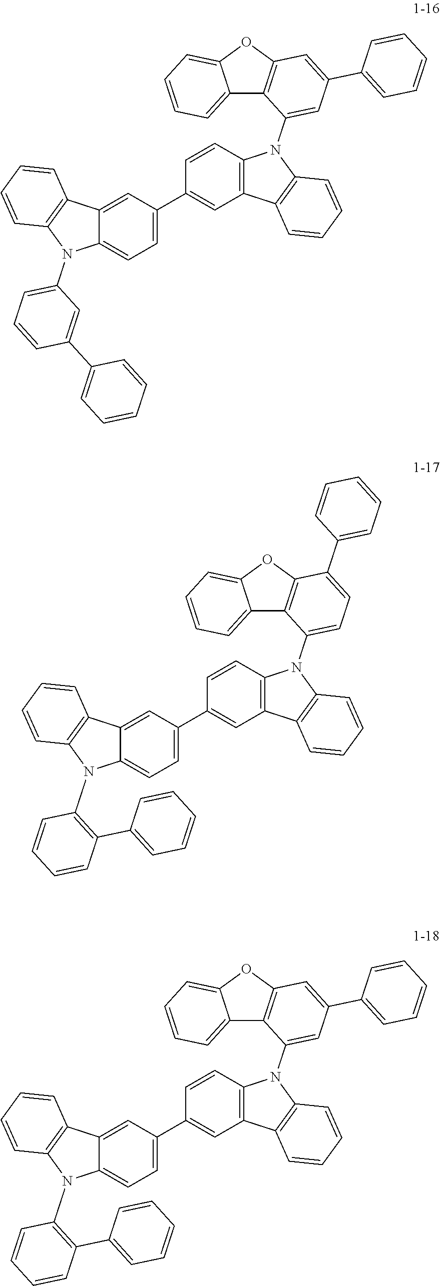 [SMILES: c1ccc(-c2cccc(-n3c4ccccc4c4cc(-c5ccc6c(c5)c5ccccc5n6-c5cc(-c6ccccc6)cc6oc7ccccc7c56)ccc43)c2)cc1 ; c1ccc(-c2ccccc2-n2c3ccccc3c3cc(-c4ccc5c(c4)c4ccccc4n5-c4ccc(-c5ccccc5)c5oc6ccccc6c45)ccc32)cc1 ; c1ccc(-c2cc(-n3c4ccccc4c4cc(-c5ccc6c(c5)c5ccccc5n6-c5ccccc5-c5ccccc5)ccc43)c3c(c2)oc2ccccc23)cc1]

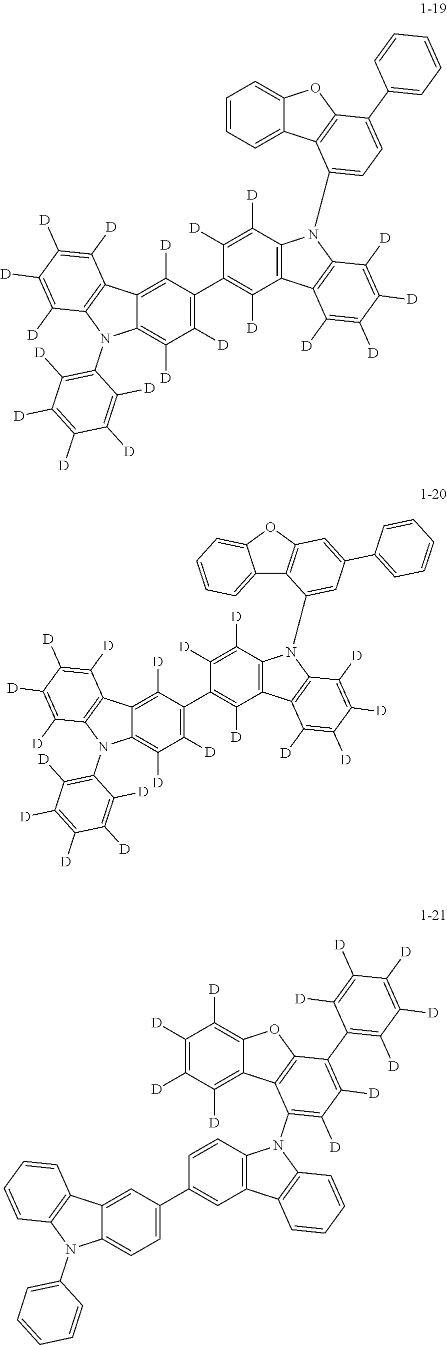

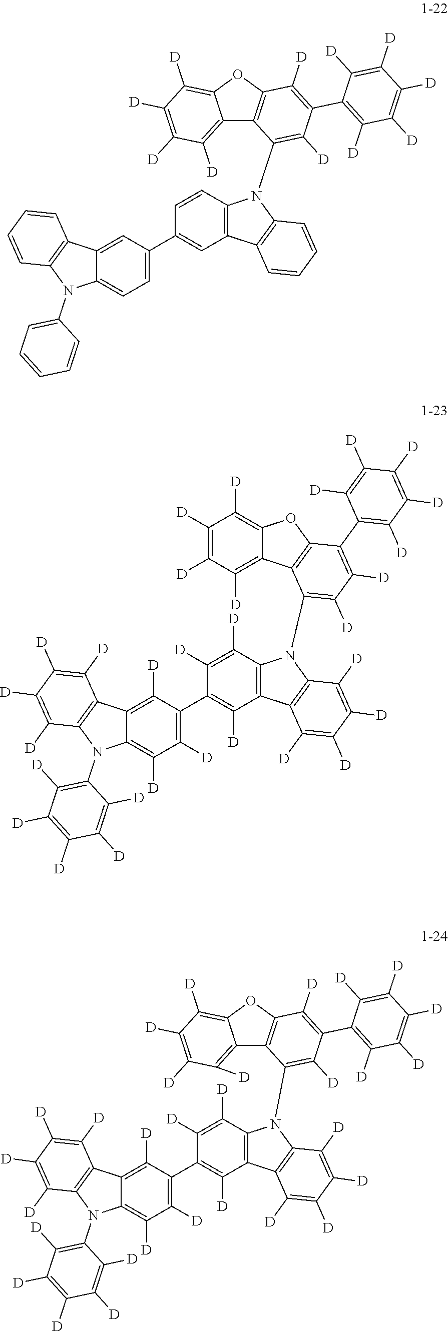

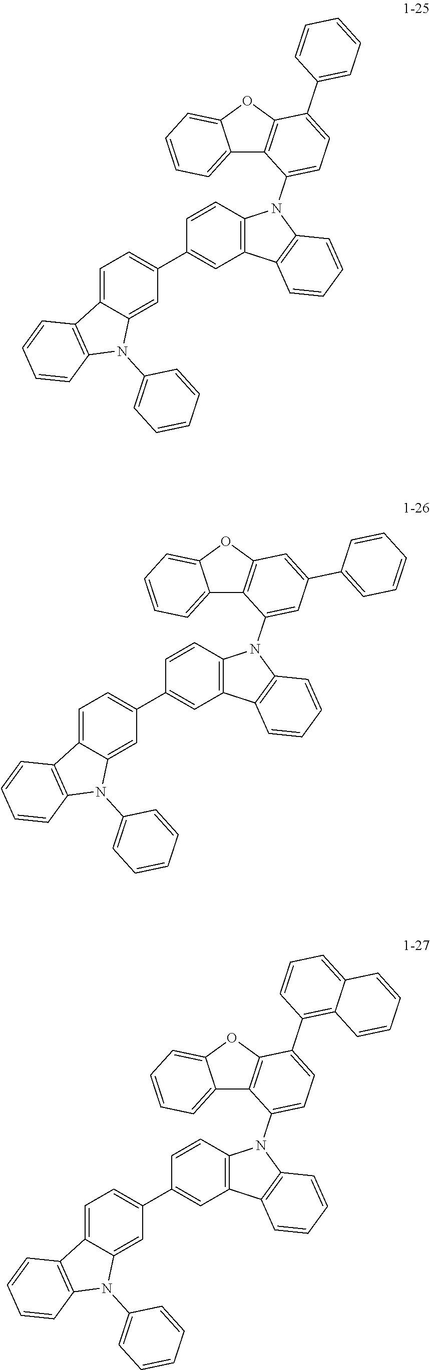 [SMILES: c1ccc(-c2ccc(-n3c4ccccc4c4cc(-c5ccc6c7ccccc7n(-c7ccccc7)c6c5)ccc43)c3c2oc2ccccc23)cc1 ; c1ccc(-c2cc(-n3c4ccccc4c4cc(-c5ccc6c7ccccc7n(-c7ccccc7)c6c5)ccc43)c3c(c2)oc2ccccc23)cc1 ; c1ccc(-n2c3ccccc3c3ccc(-c4ccc5c(c4)c4ccccc4n5-c4ccc(-c5cccc6ccccc56)c5oc6ccccc6c45)cc32)cc1]

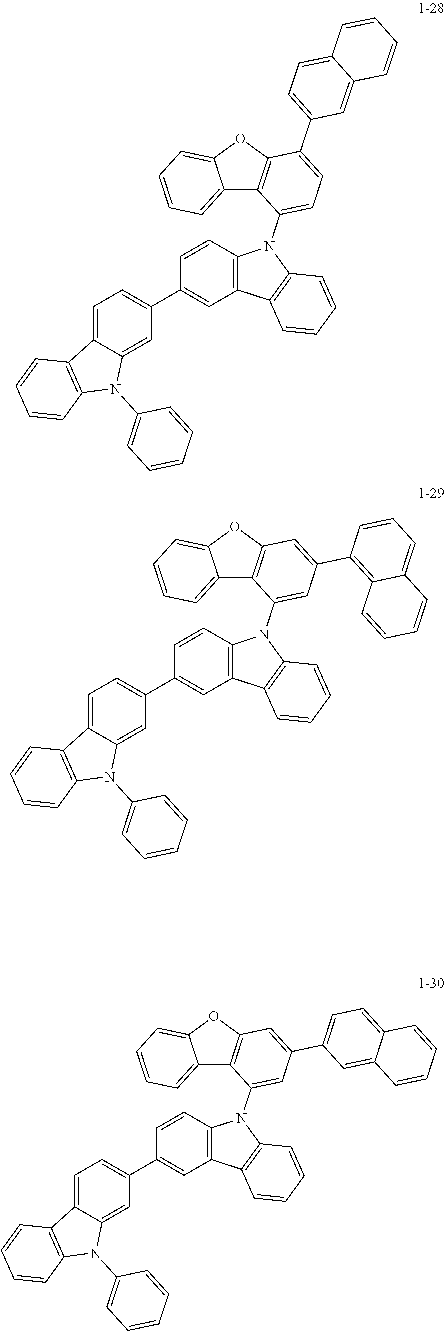 [SMILES: c1ccc(-n2c3ccccc3c3ccc(-c4ccc5c(c4)c4ccccc4n5-c4ccc(-c5ccc6ccccc6c5)c5oc6ccccc6c45)cc32)cc1 ; c1ccc(-n2c3ccccc3c3ccc(-c4ccc5c(c4)c4ccccc4n5-c4cc(-c5cccc6ccccc56)cc5oc6ccccc6c45)cc32)cc1 ; c1ccc(-n2c3ccccc3c3ccc(-c4ccc5c(c4)c4ccccc4n5-c4cc(-c5ccc6ccccc6c5)cc5oc6ccccc6c45)cc32)cc1]

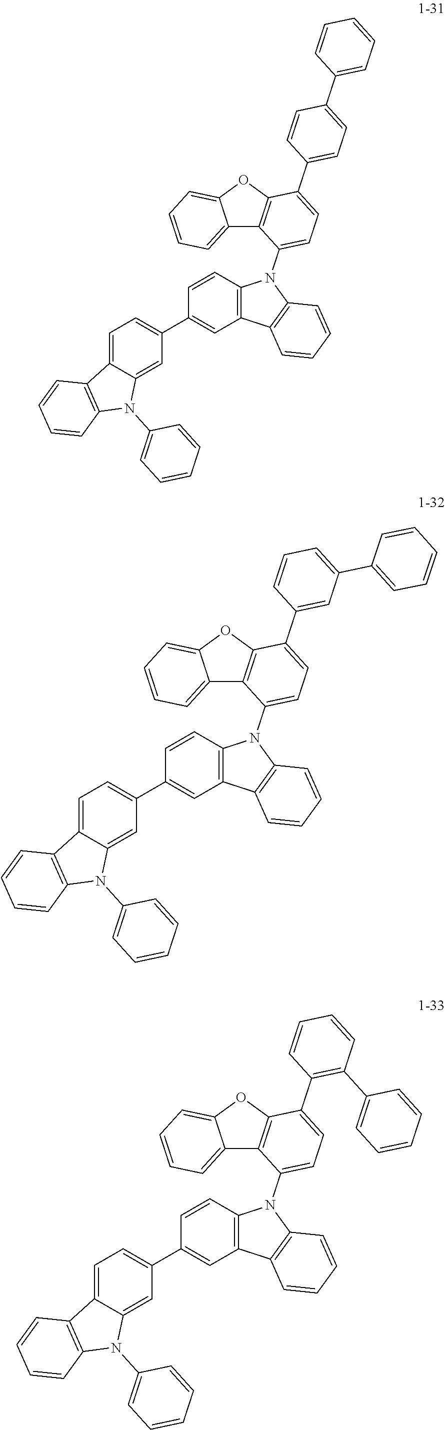 [SMILES: c1ccc(-c2ccc(-c3ccc(-n4c5ccccc5c5cc(-c6ccc7c8ccccc8n(-c8ccccc8)c7c6)ccc54)c4c3oc3ccccc34)cc2)cc1 ; c1ccc(-c2cccc(-c3ccc(-n4c5ccccc5c5cc(-c6ccc7c8ccccc8n(-c8ccccc8)c7c6)ccc54)c4c3oc3ccccc34)c2)cc1 ; c1ccc(-c2ccccc2-c2ccc(-n3c4ccccc4c4cc(-c5ccc6c7ccccc7n(-c7ccccc7)c6c5)ccc43)c3c2oc2ccccc23)cc1]

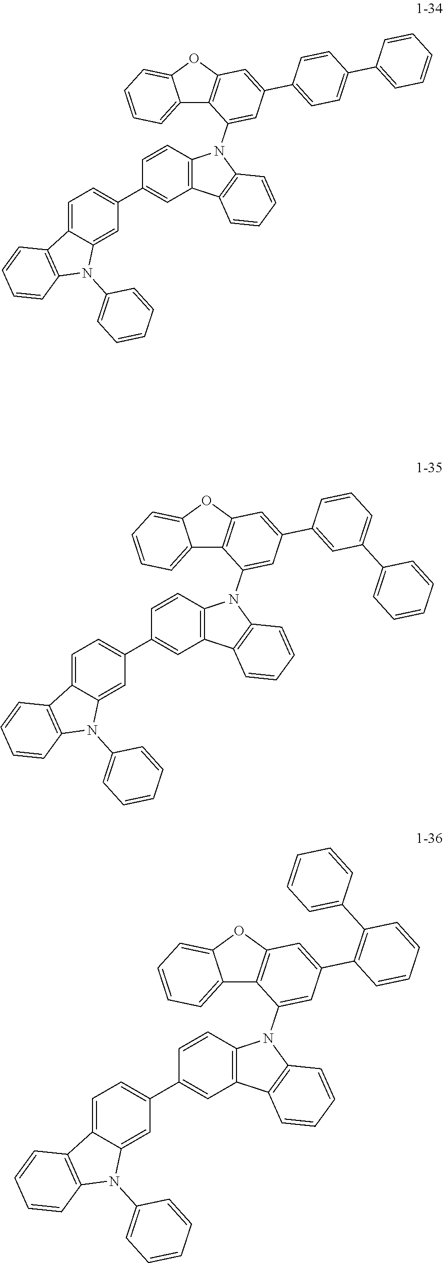 [SMILES: c1ccc(-c2ccc(-c3cc(-n4c5ccccc5c5cc(-c6ccc7c8ccccc8n(-c8ccccc8)c7c6)ccc54)c4c(c3)oc3ccccc34)cc2)cc1 ; c1ccc(-c2cccc(-c3cc(-n4c5ccccc5c5cc(-c6ccc7c8ccccc8n(-c8ccccc8)c7c6)ccc54)c4c(c3)oc3ccccc34)c2)cc1 ; c1ccc(-c2ccccc2-c2cc(-n3c4ccccc4c4cc(-c5ccc6c7ccccc7n(-c7ccccc7)c6c5)ccc43)c3c(c2)oc2ccccc23)cc1]

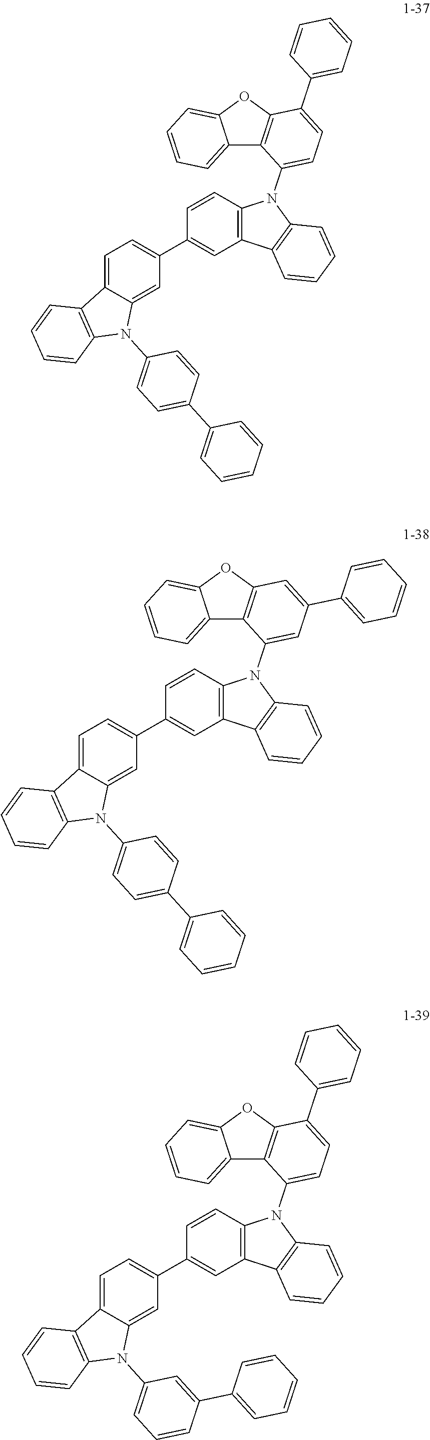 [SMILES: c1ccc(-c2ccc(-n3c4ccccc4c4ccc(-c5ccc6c(c5)c5ccccc5n6-c5ccc(-c6ccccc6)c6oc7ccccc7c56)cc43)cc2)cc1 ; c1ccc(-c2ccc(-n3c4ccccc4c4ccc(-c5ccc6c(c5)c5ccccc5n6-c5cc(-c6ccccc6)cc6oc7ccccc7c56)cc43)cc2)cc1 ; c1ccc(-c2cccc(-n3c4ccccc4c4ccc(-c5ccc6c(c5)c5ccccc5n6-c5ccc(-c6ccccc6)c6oc7ccccc7c56)cc43)c2)cc1]

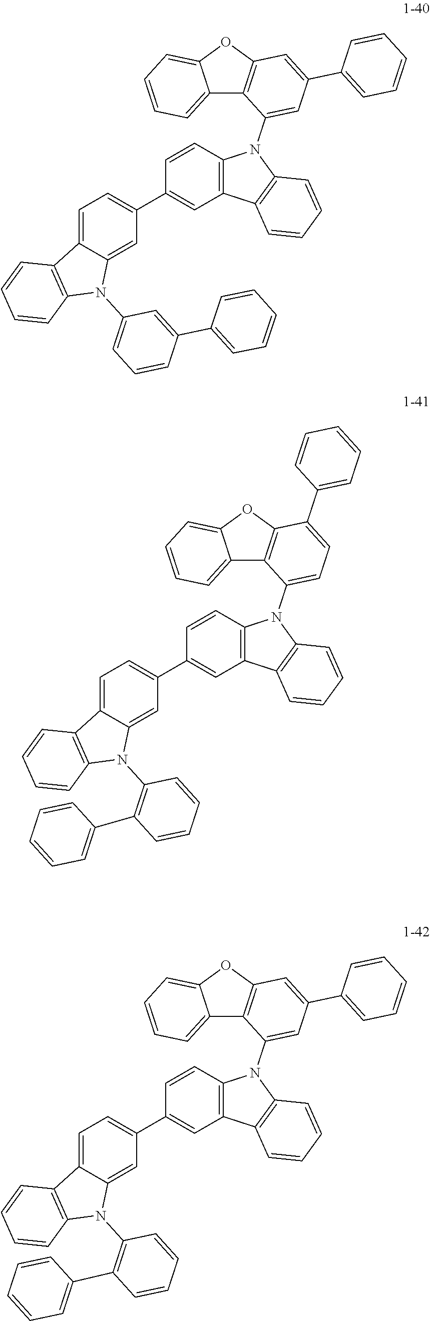 [SMILES: c1ccc(-c2cccc(-n3c4ccccc4c4ccc(-c5ccc6c(c5)c5ccccc5n6-c5cc(-c6ccccc6)cc6oc7ccccc7c56)cc43)c2)cc1 ; c1ccc(-c2ccccc2-n2c3ccccc3c3ccc(-c4ccc5c(c4)c4ccccc4n5-c4ccc(-c5ccccc5)c5oc6ccccc6c45)cc32)cc1 ; c1ccc(-c2cc(-n3c4ccccc4c4cc(-c5ccc6c7ccccc7n(-c7ccccc7-c7ccccc7)c6c5)ccc43)c3c(c2)oc2ccccc23)cc1]

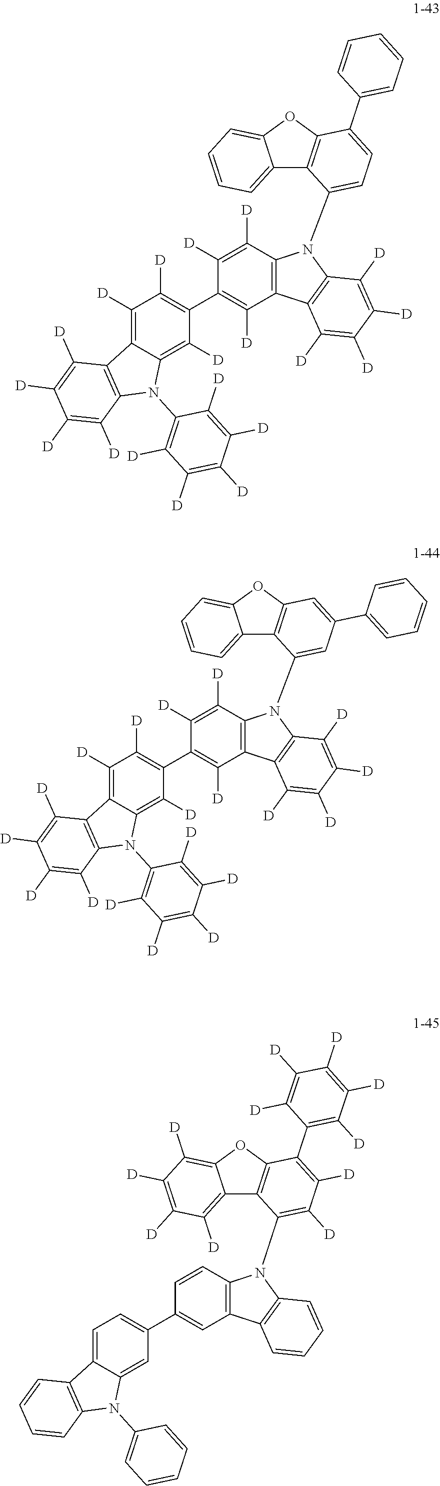

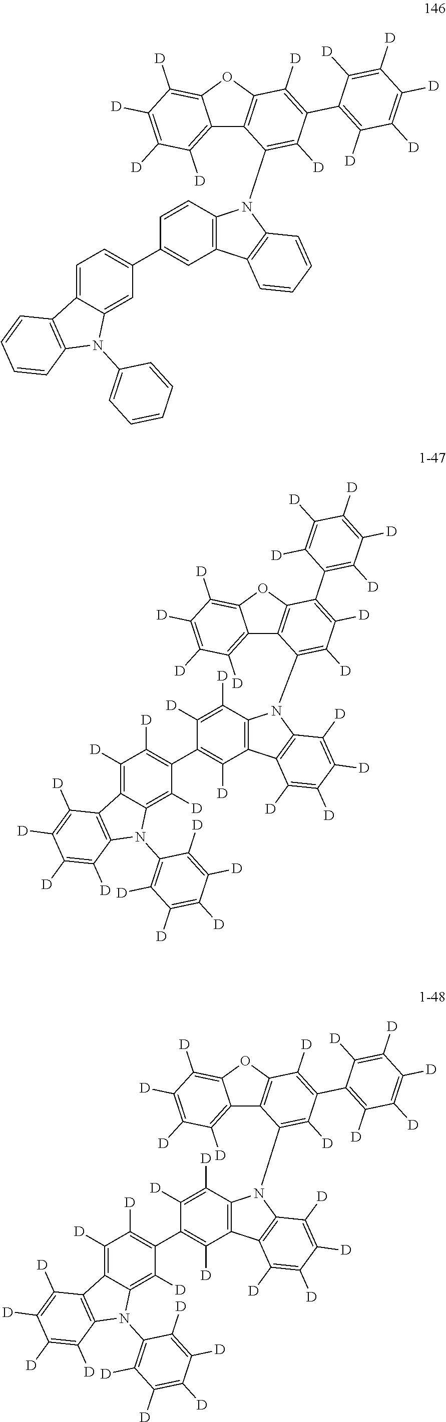

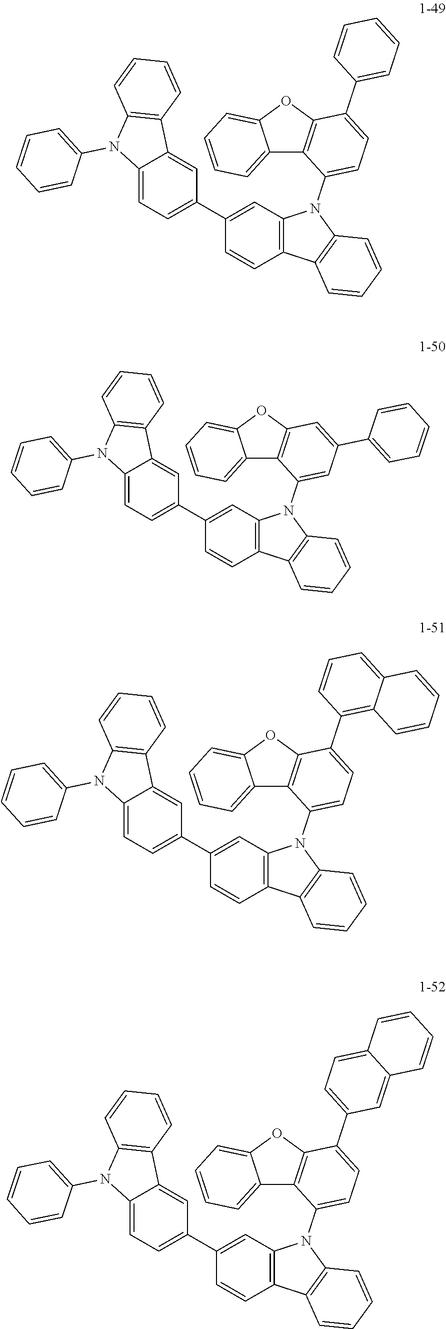 [SMILES: c1ccc(-c2ccc(-n3c4ccccc4c4ccc(-c5ccc6c(c5)c5ccccc5n6-c5ccccc5)cc43)c3c2oc2ccccc23)cc1 ; c1ccc(-c2cc(-n3c4ccccc4c4ccc(-c5ccc6c(c5)c5ccccc5n6-c5ccccc5)cc43)c3c(c2)oc2ccccc23)cc1 ; c1ccc(-n2c3ccccc3c3cc(-c4ccc5c6ccccc6n(-c6ccc(-c7cccc8ccccc78)c7oc8ccccc8c67)c5c4)ccc32)cc1 ; c1ccc(-n2c3ccccc3c3cc(-c4ccc5c6ccccc6n(-c6ccc(-c7ccc8ccccc8c7)c7oc8ccccc8c67)c5c4)ccc32)cc1]

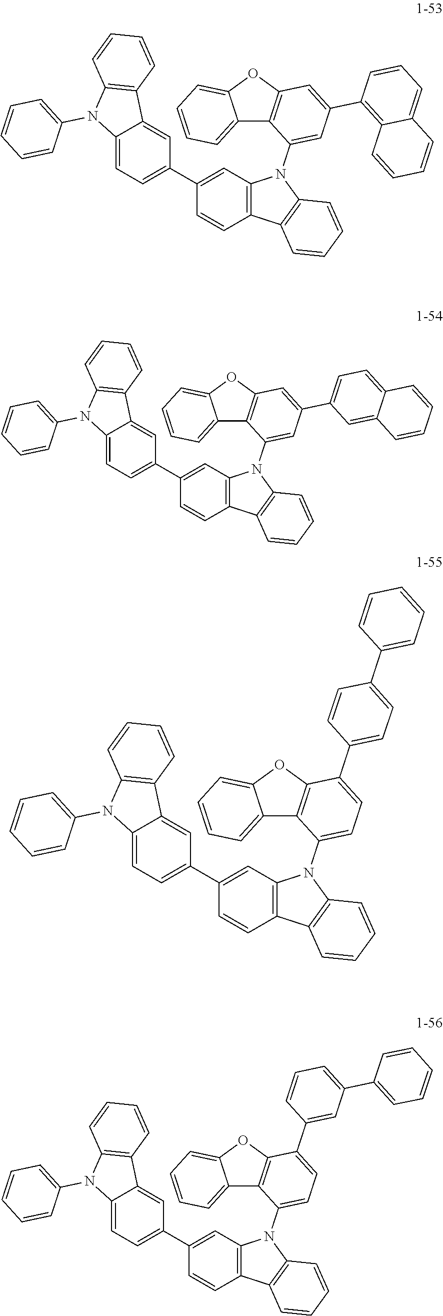 [SMILES: c1ccc(-n2c3ccccc3c3cc(-c4ccc5c6ccccc6n(-c6cc(-c7cccc8ccccc78)cc7oc8ccccc8c67)c5c4)ccc32)cc1 ; c1ccc(-n2c3ccccc3c3cc(-c4ccc5c6ccccc6n(-c6cc(-c7ccc8ccccc8c7)cc7oc8ccccc8c67)c5c4)ccc32)cc1 ; c1ccc(-c2ccc(-c3ccc(-n4c5ccccc5c5ccc(-c6ccc7c(c6)c6ccccc6n7-c6ccccc6)cc54)c4c3oc3ccccc34)cc2)cc1 ; c1ccc(-c2cccc(-c3ccc(-n4c5ccccc5c5ccc(-c6ccc7c(c6)c6ccccc6n7-c6ccccc6)cc54)c4c3oc3ccccc34)c2)cc1]

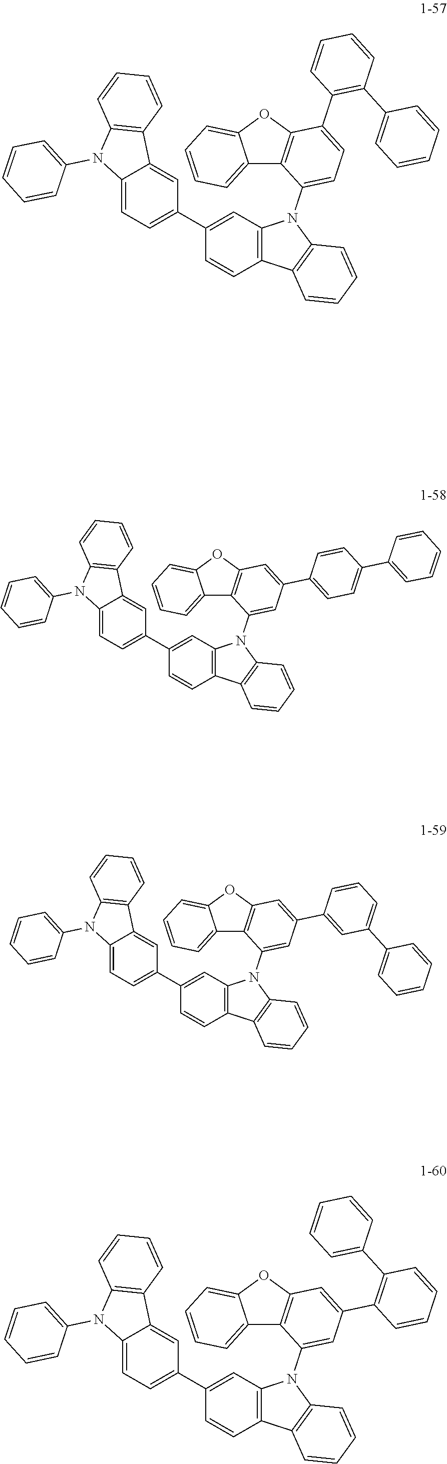 [SMILES: c1ccc(-c2ccccc2-c2ccc(-n3c4ccccc4c4ccc(-c5ccc6c(c5)c5ccccc5n6-c5ccccc5)cc43)c3c2oc2ccccc23)cc1 ; c1ccc(-c2ccc(-c3cc(-n4c5ccccc5c5ccc(-c6ccc7c(c6)c6ccccc6n7-c6ccccc6)cc54)c4c(c3)oc3ccccc34)cc2)cc1 ; c1ccc(-c2cccc(-c3cc(-n4c5ccccc5c5ccc(-c6ccc7c(c6)c6ccccc6n7-c6ccccc6)cc54)c4c(c3)oc3ccccc34)c2)cc1 ; c1ccc(-c2ccccc2-c2cc(-n3c4ccccc4c4ccc(-c5ccc6c(c5)c5ccccc5n6-c5ccccc5)cc43)c3c(c2)oc2ccccc23)cc1]

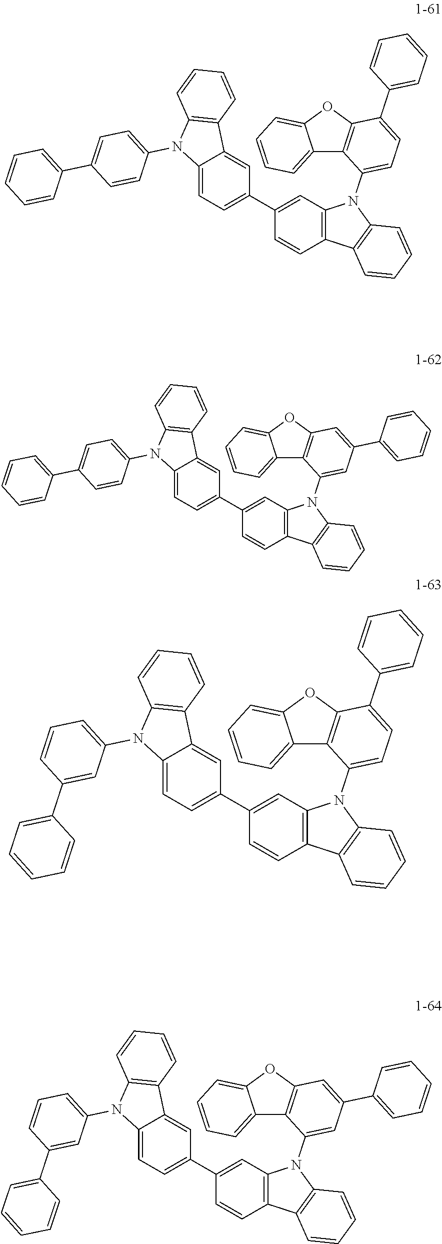 [SMILES: c1ccc(-c2ccc(-n3c4ccccc4c4cc(-c5ccc6c7ccccc7n(-c7ccc(-c8ccccc8)c8oc9ccccc9c78)c6c5)ccc43)cc2)cc1 ; c1ccc(-c2ccc(-n3c4ccccc4c4cc(-c5ccc6c7ccccc7n(-c7cc(-c8ccccc8)cc8oc9ccccc9c78)c6c5)ccc43)cc2)cc1 ; c1ccc(-c2cccc(-n3c4ccccc4c4cc(-c5ccc6c7ccccc7n(-c7ccc(-c8ccccc8)c8oc9ccccc9c78)c6c5)ccc43)c2)cc1 ; c1ccc(-c2cccc(-n3c4ccccc4c4cc(-c5ccc6c7ccccc7n(-c7cc(-c8ccccc8)cc8oc9ccccc9c78)c6c5)ccc43)c2)cc1]

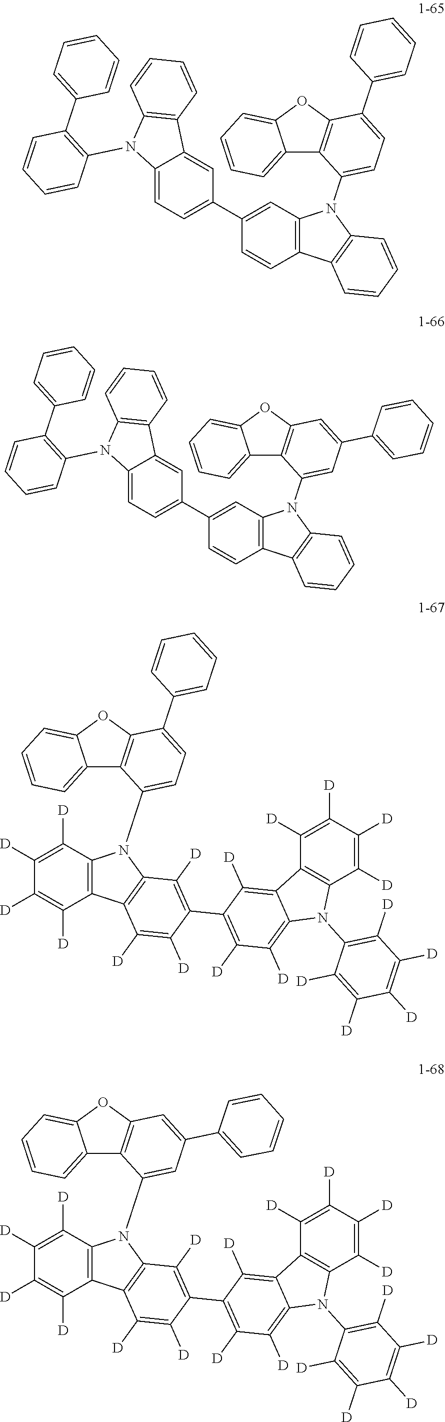

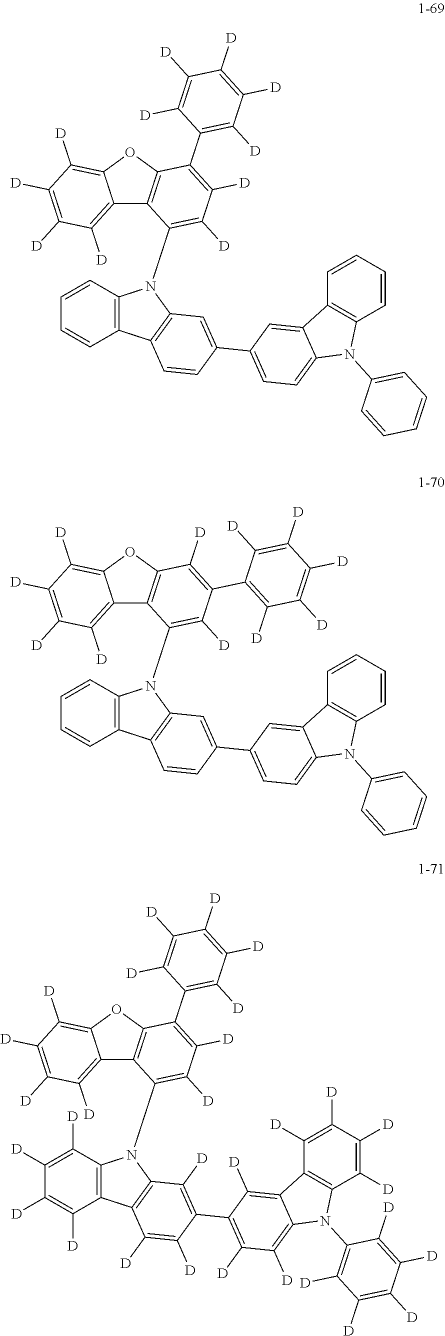

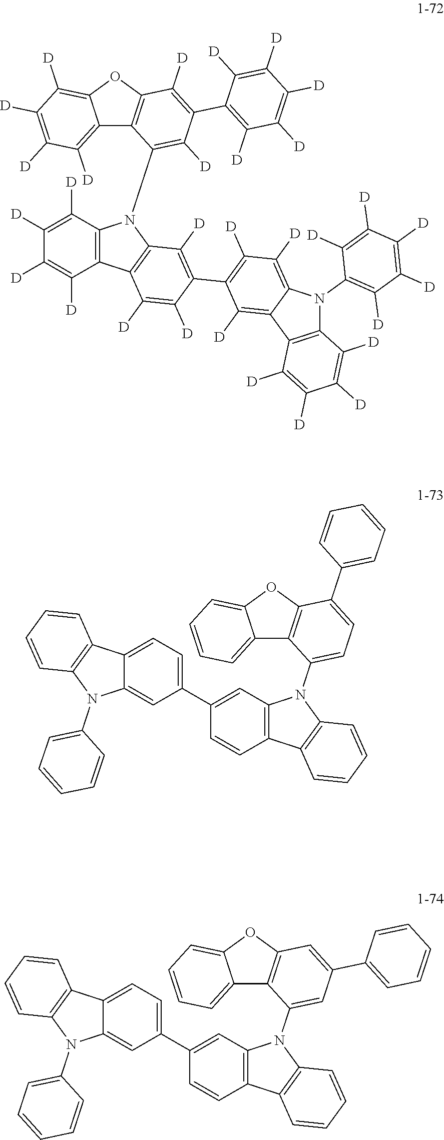 [SMILES: [2H]c1c([2H])c([2H])c(-c2c([2H])c(-n3c4c([2H])c([2H])c([2H])c([2H])c4c4c([2H])c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c43)c3c(oc4c([2H])c([2H])c([2H])c([2H])c43)c2[2H])c([2H])c1[2H] ; c1ccc(-c2ccc(-n3c4ccccc4c4ccc(-c5ccc6c7ccccc7n(-c7ccccc7)c6c5)cc43)c3c2oc2ccccc23)cc1 ; c1ccc(-c2cc(-n3c4ccccc4c4ccc(-c5ccc6c7ccccc7n(-c7ccccc7)c6c5)cc43)c3c(c2)oc2ccccc23)cc1]

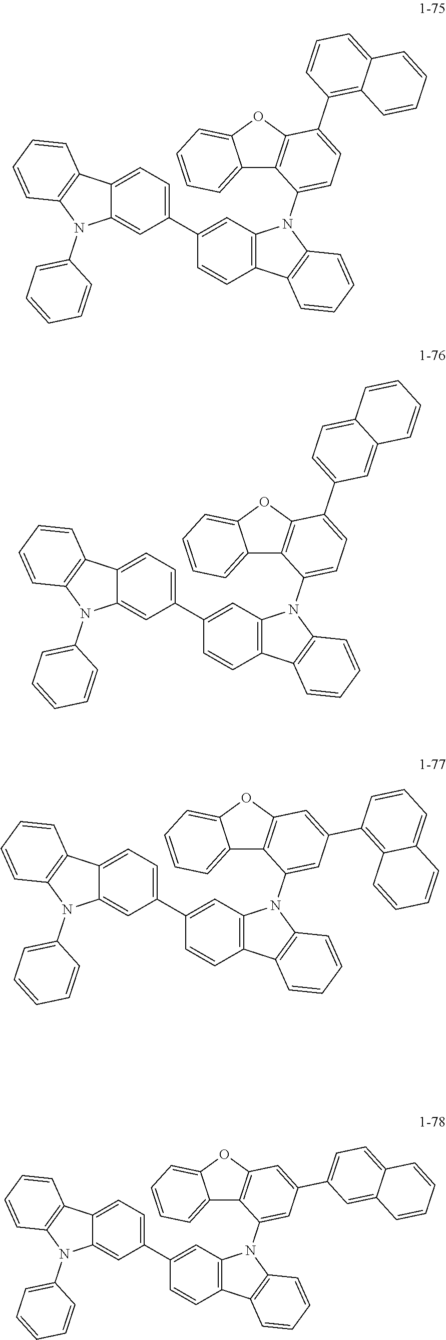 [SMILES: c1ccc(-n2c3ccccc3c3ccc(-c4ccc5c6ccccc6n(-c6ccc(-c7cccc8ccccc78)c7oc8ccccc8c67)c5c4)cc32)cc1 ; c1ccc(-n2c3ccccc3c3ccc(-c4ccc5c6ccccc6n(-c6ccc(-c7ccc8ccccc8c7)c7oc8ccccc8c67)c5c4)cc32)cc1 ; c1ccc(-n2c3ccccc3c3ccc(-c4ccc5c6ccccc6n(-c6cc(-c7cccc8ccccc78)cc7oc8ccccc8c67)c5c4)cc32)cc1 ; c1ccc(-n2c3ccccc3c3ccc(-c4ccc5c6ccccc6n(-c6cc(-c7ccc8ccccc8c7)cc7oc8ccccc8c67)c5c4)cc32)cc1]

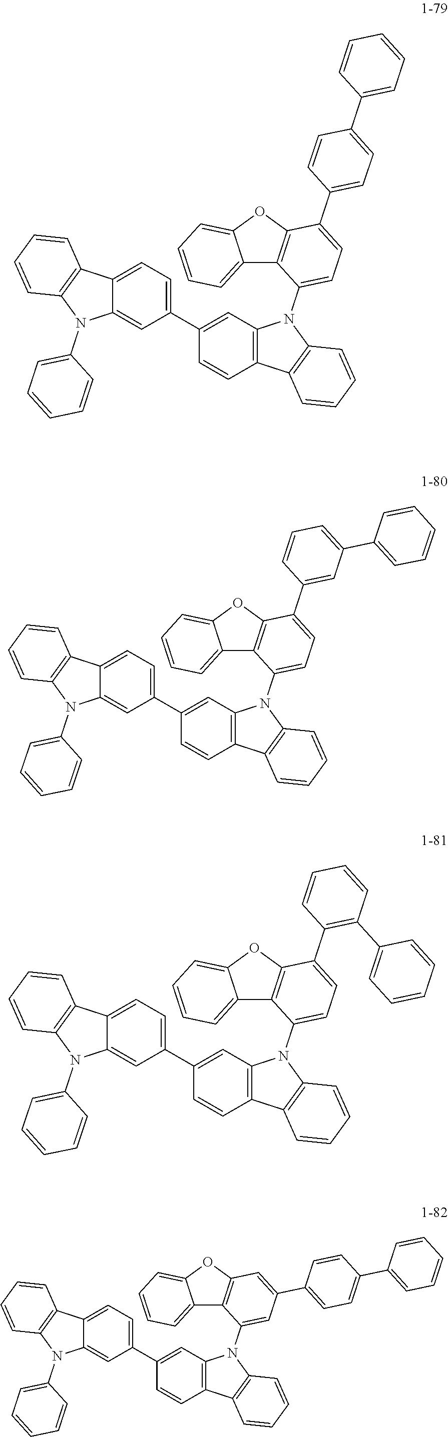 [SMILES: c1ccc(-c2ccc(-c3ccc(-n4c5ccccc5c5ccc(-c6ccc7c8ccccc8n(-c8ccccc8)c7c6)cc54)c4c3oc3ccccc34)cc2)cc1 ; c1ccc(-c2cccc(-c3ccc(-n4c5ccccc5c5ccc(-c6ccc7c8ccccc8n(-c8ccccc8)c7c6)cc54)c4c3oc3ccccc34)c2)cc1 ; c1ccc(-c2ccccc2-c2ccc(-n3c4ccccc4c4ccc(-c5ccc6c7ccccc7n(-c7ccccc7)c6c5)cc43)c3c2oc2ccccc23)cc1 ; c1ccc(-c2ccc(-c3cc(-n4c5ccccc5c5ccc(-c6ccc7c8ccccc8n(-c8ccccc8)c7c6)cc54)c4c(c3)oc3ccccc34)cc2)cc1]

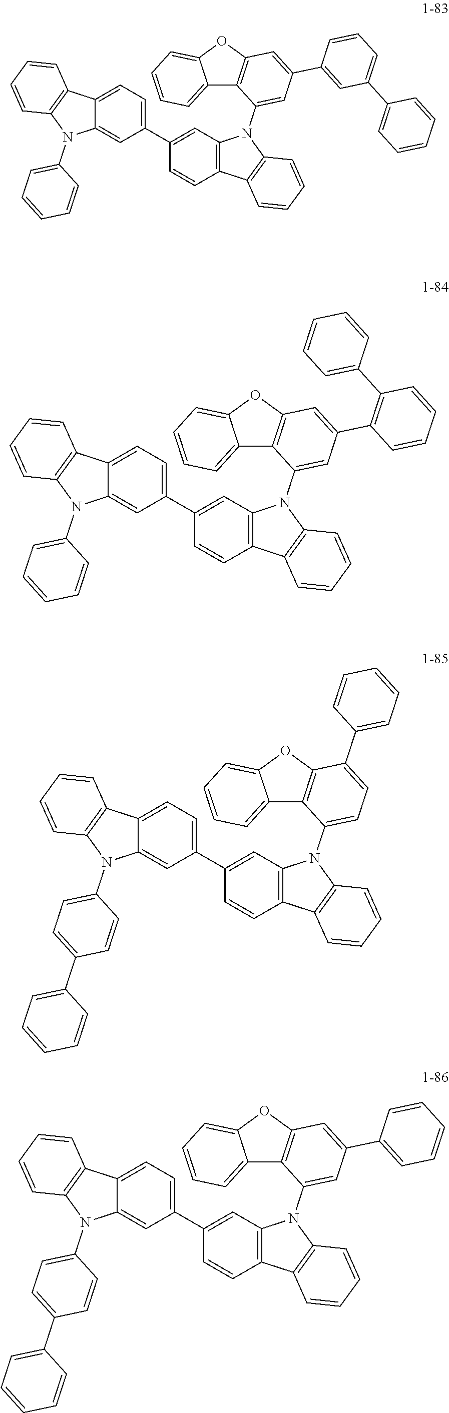 [SMILES: c1ccc(-c2cccc(-c3cc(-n4c5ccccc5c5ccc(-c6ccc7c8ccccc8n(-c8ccccc8)c7c6)cc54)c4c(c3)oc3ccccc34)c2)cc1 ; c1ccc(-c2ccccc2-c2cc(-n3c4ccccc4c4ccc(-c5ccc6c7ccccc7n(-c7ccccc7)c6c5)cc43)c3c(c2)oc2ccccc23)cc1 ; c1ccc(-c2ccc(-n3c4ccccc4c4ccc(-c5ccc6c7ccccc7n(-c7ccc(-c8ccccc8)c8oc9ccccc9c78)c6c5)cc43)cc2)cc1 ; c1ccc(-c2ccc(-n3c4ccccc4c4ccc(-c5ccc6c7ccccc7n(-c7cc(-c8ccccc8)cc8oc9ccccc9c78)c6c5)cc43)cc2)cc1]

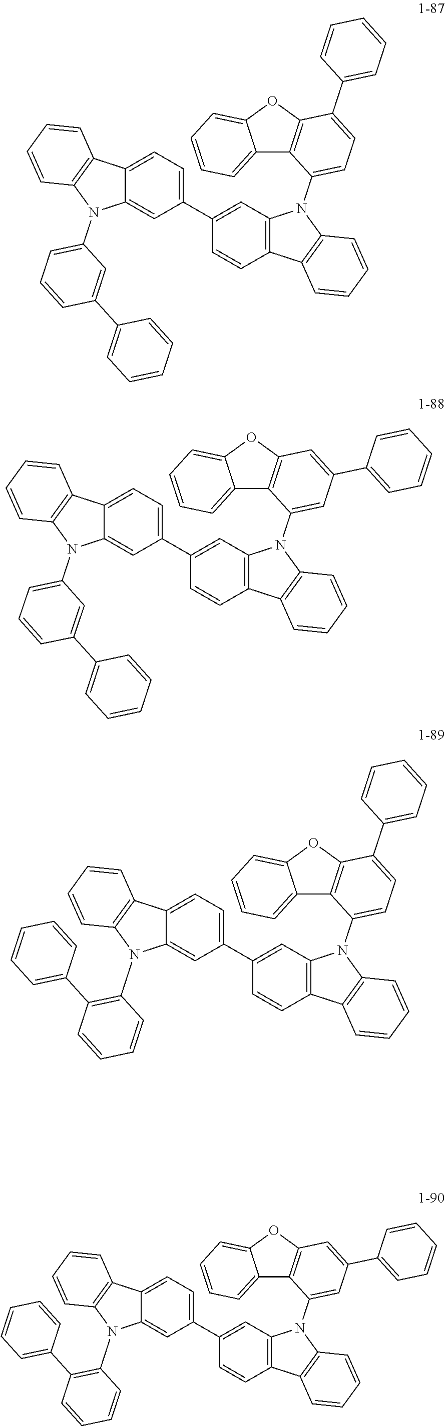 [SMILES: c1ccc(-c2cccc(-n3c4ccccc4c4ccc(-c5ccc6c7ccccc7n(-c7ccc(-c8ccccc8)c8oc9ccccc9c78)c6c5)cc43)c2)cc1 ; c1ccc(-c2cccc(-n3c4ccccc4c4ccc(-c5ccc6c7ccccc7n(-c7cc(-c8ccccc8)cc8oc9ccccc9c78)c6c5)cc43)c2)cc1 ; c1ccc(-c2ccccc2-n2c3ccccc3c3ccc(-c4ccc5c6ccccc6n(-c6ccc(-c7ccccc7)c7oc8ccccc8c67)c5c4)cc32)cc1 ; c1ccc(-c2cc(-n3c4ccccc4c4ccc(-c5ccc6c7ccccc7n(-c7ccccc7-c7ccccc7)c6c5)cc43)c3c(c2)oc2ccccc23)cc1]

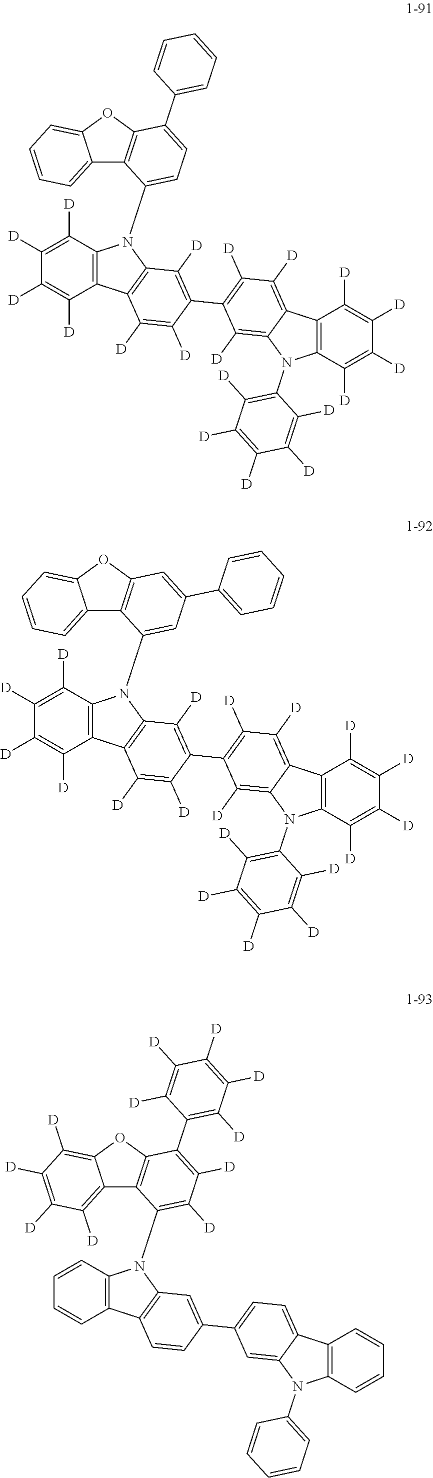

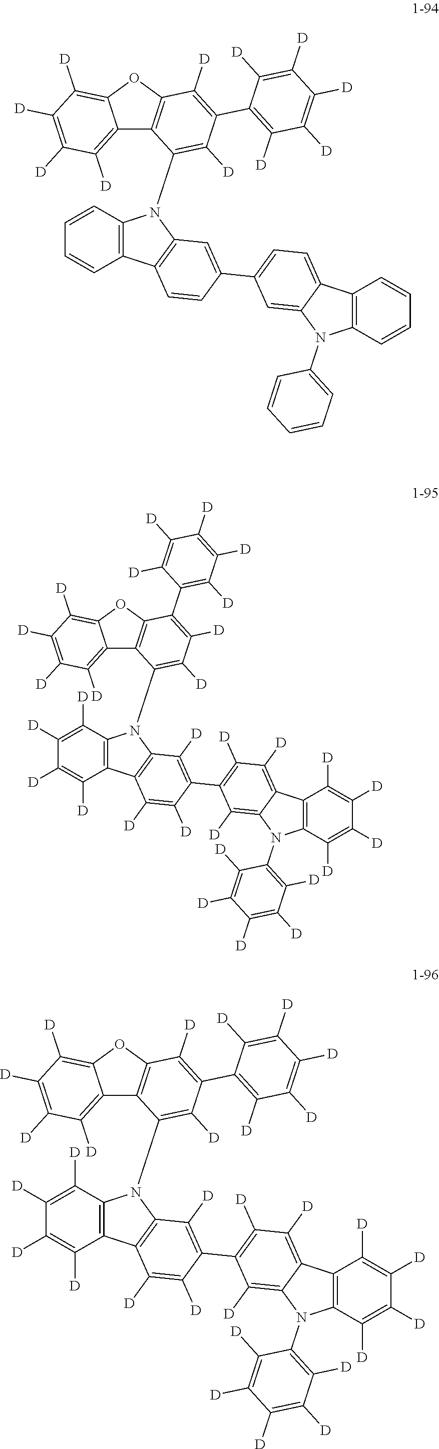

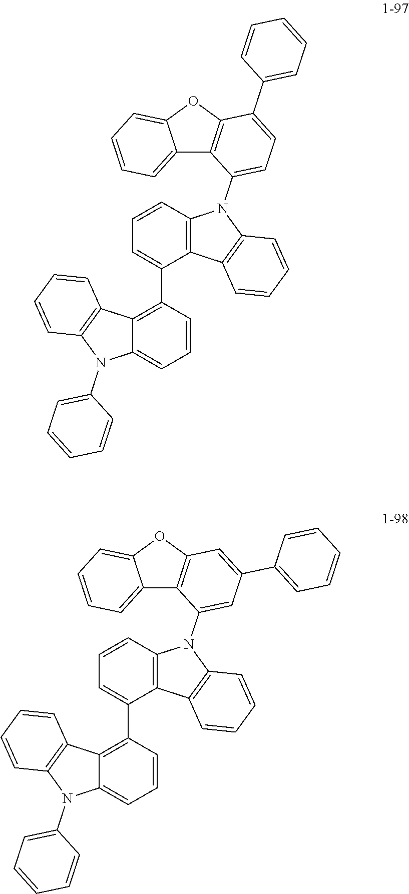 [SMILES: c1ccc(-c2ccc(-n3c4ccccc4c4c(-c5cccc6c5c5ccccc5n6-c5ccccc5)cccc43)c3c2oc2ccccc23)cc1 ; c1ccc(-c2cc(-n3c4ccccc4c4c(-c5cccc6c5c5ccccc5n6-c5ccccc5)cccc43)c3c(c2)oc2ccccc23)cc1]

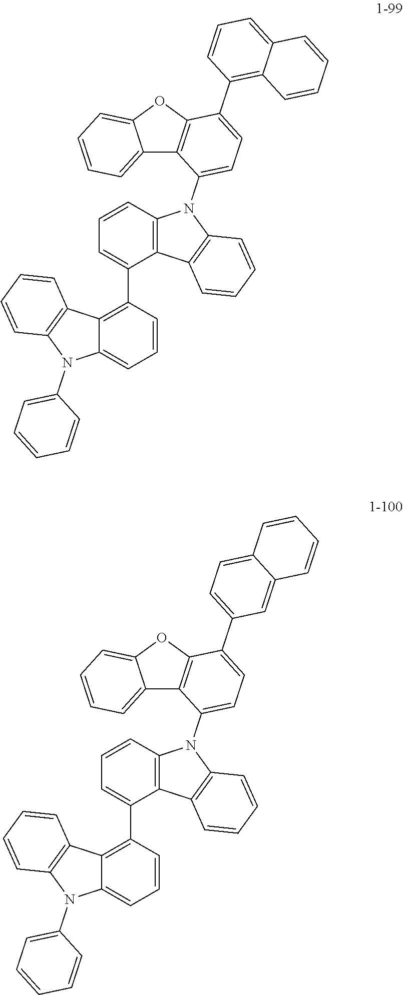 [SMILES: c1ccc(-n2c3ccccc3c3c(-c4cccc5c4c4ccccc4n5-c4ccc(-c5cccc6ccccc56)c5oc6ccccc6c45)cccc32)cc1 ; c1ccc(-n2c3ccccc3c3c(-c4cccc5c4c4ccccc4n5-c4ccc(-c5ccc6ccccc6c5)c5oc6ccccc6c45)cccc32)cc1]

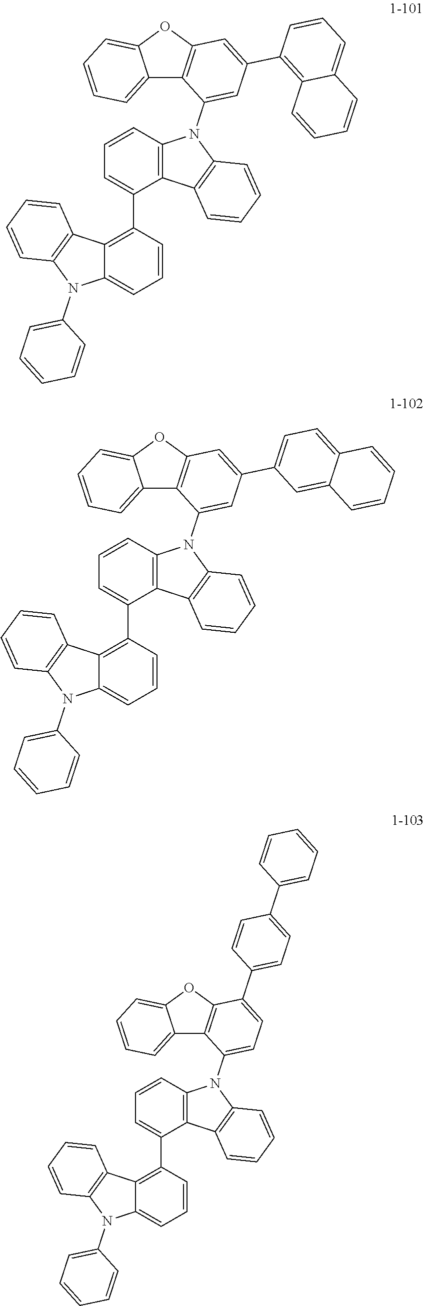 [SMILES: c1ccc(-n2c3ccccc3c3c(-c4cccc5c4c4ccccc4n5-c4cc(-c5cccc6ccccc56)cc5oc6ccccc6c45)cccc32)cc1 ; c1ccc(-n2c3ccccc3c3c(-c4cccc5c4c4ccccc4n5-c4cc(-c5ccc6ccccc6c5)cc5oc6ccccc6c45)cccc32)cc1 ; c1ccc(-c2ccc(-c3ccc(-n4c5ccccc5c5c(-c6cccc7c6c6ccccc6n7-c6ccccc6)cccc54)c4c3oc3ccccc34)cc2)cc1]

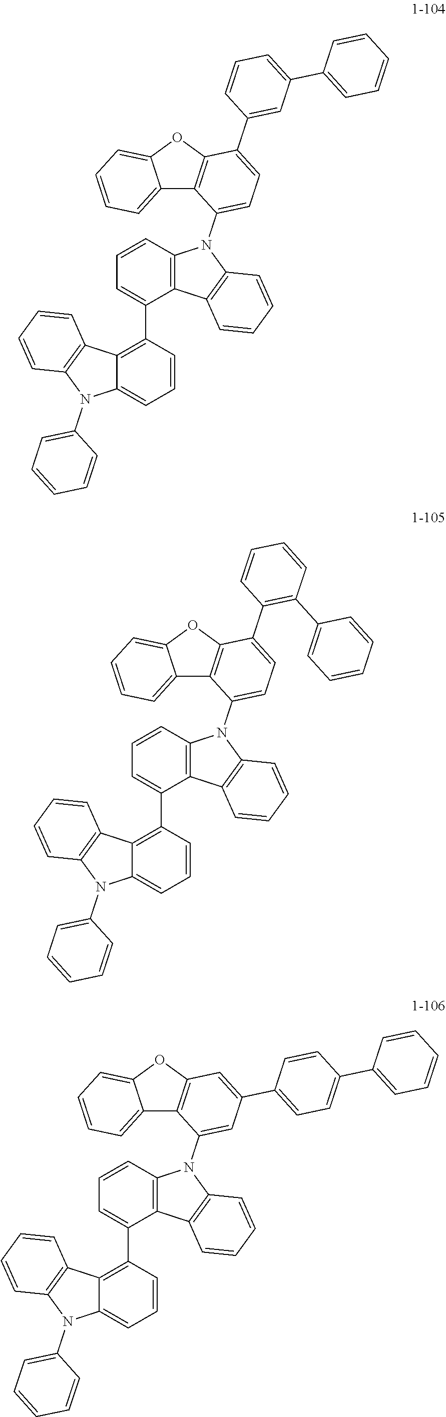 [SMILES: c1ccc(-c2cccc(-c3ccc(-n4c5ccccc5c5c(-c6cccc7c6c6ccccc6n7-c6ccccc6)cccc54)c4c3oc3ccccc34)c2)cc1 ; c1ccc(-c2ccccc2-c2ccc(-n3c4ccccc4c4c(-c5cccc6c5c5ccccc5n6-c5ccccc5)cccc43)c3c2oc2ccccc23)cc1 ; c1ccc(-c2ccc(-c3cc(-n4c5ccccc5c5c(-c6cccc7c6c6ccccc6n7-c6ccccc6)cccc54)c4c(c3)oc3ccccc34)cc2)cc1]

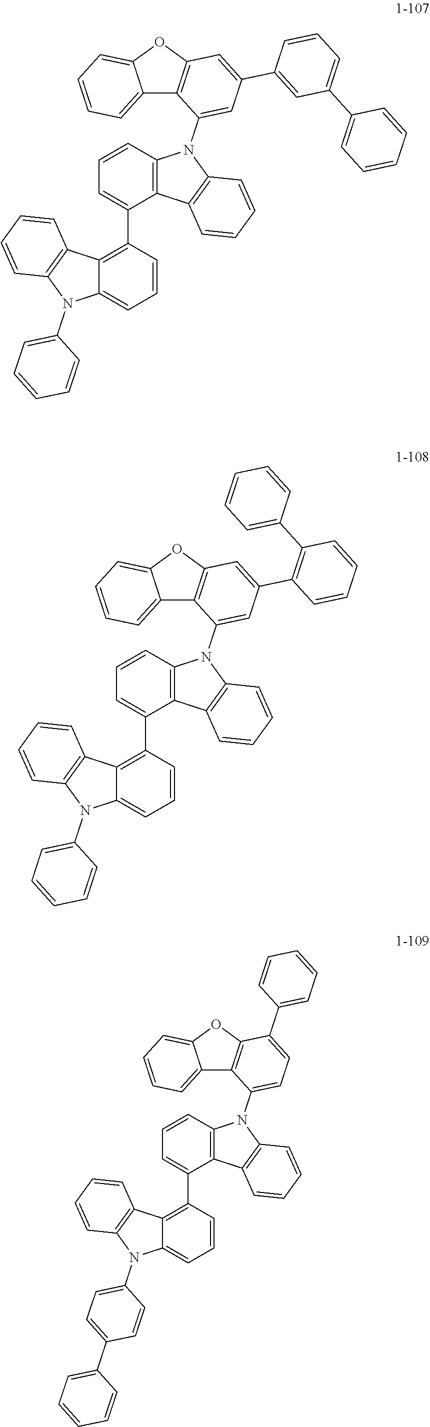 [SMILES: c1ccc(-c2cccc(-c3cc(-n4c5ccccc5c5c(-c6cccc7c6c6ccccc6n7-c6ccccc6)cccc54)c4c(c3)oc3ccccc34)c2)cc1 ; c1ccc(-c2ccccc2-c2cc(-n3c4ccccc4c4c(-c5cccc6c5c5ccccc5n6-c5ccccc5)cccc43)c3c(c2)oc2ccccc23)cc1 ; c1ccc(-c2ccc(-n3c4ccccc4c4c(-c5cccc6c5c5ccccc5n6-c5ccc(-c6ccccc6)c6oc7ccccc7c56)cccc43)cc2)cc1]

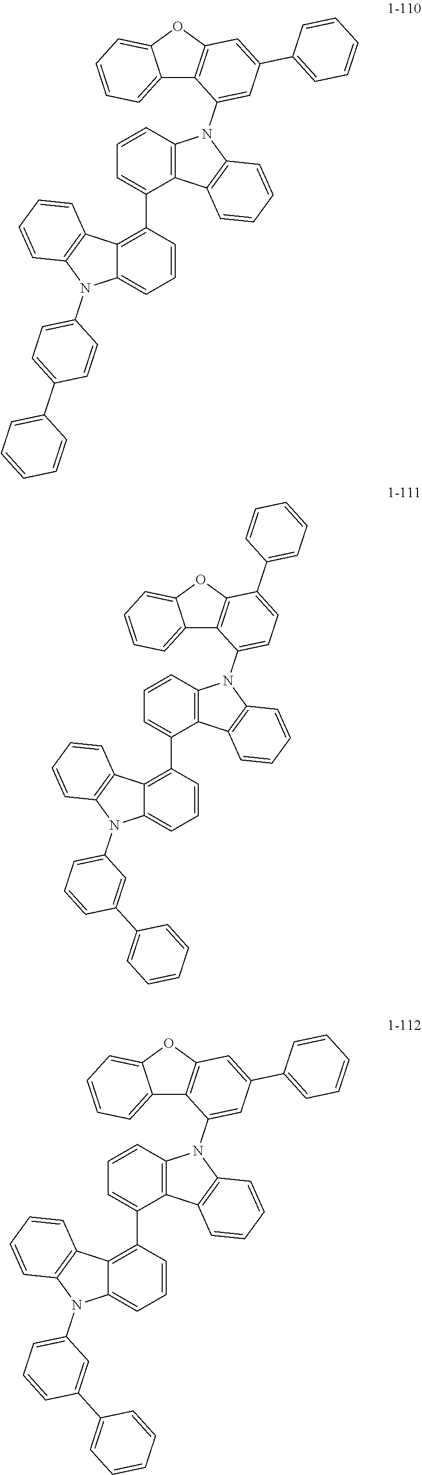 [SMILES: c1ccc(-c2ccc(-n3c4ccccc4c4c(-c5cccc6c5c5ccccc5n6-c5cc(-c6ccccc6)cc6oc7ccccc7c56)cccc43)cc2)cc1 ; c1ccc(-c2cccc(-n3c4ccccc4c4c(-c5cccc6c5c5ccccc5n6-c5ccc(-c6ccccc6)c6oc7ccccc7c56)cccc43)c2)cc1 ; c1ccc(-c2cccc(-n3c4ccccc4c4c(-c5cccc6c5c5ccccc5n6-c5cc(-c6ccccc6)cc6oc7ccccc7c56)cccc43)c2)cc1]

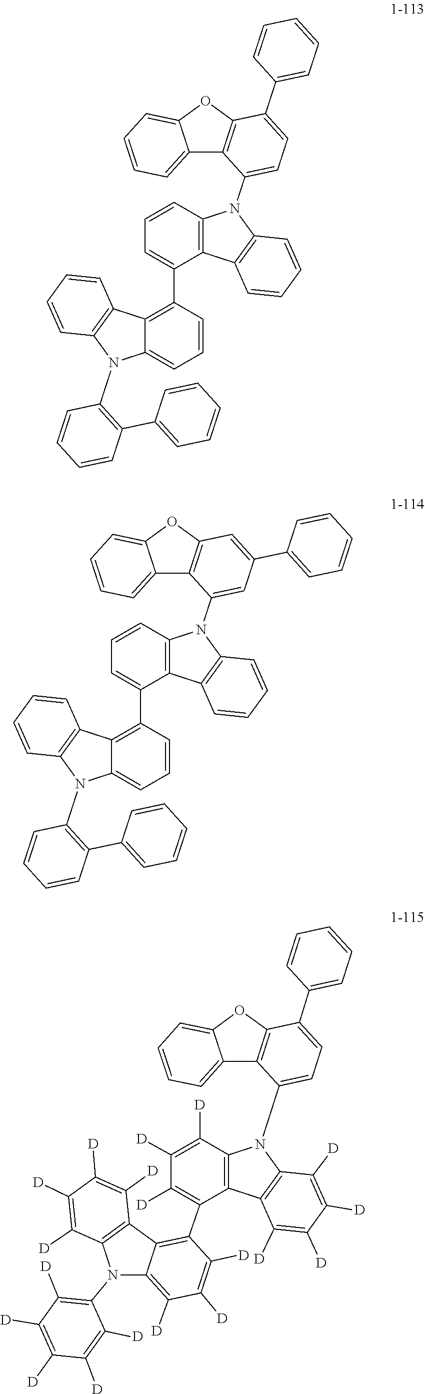 [SMILES: c1ccc(-c2ccccc2-n2c3ccccc3c3c(-c4cccc5c4c4ccccc4n5-c4ccc(-c5ccccc5)c5oc6ccccc6c45)cccc32)cc1 ; c1ccc(-c2cc(-n3c4ccccc4c4c(-c5cccc6c5c5ccccc5n6-c5ccccc5-c5ccccc5)cccc43)c3c(c2)oc2ccccc23)cc1 ; [2H]c1c([2H])c([2H])c(-n2c3c([2H])c([2H])c([2H])c([2H])c3c3c(-c4c([2H])c([2H])c([2H])c5c4c4c([2H])c([2H])c([2H])c([2H])c4n5-c4ccc(-c5ccccc5)c5oc6ccccc6c45)c([2H])c([2H])c([2H])c32)c([2H])c1[2H]]

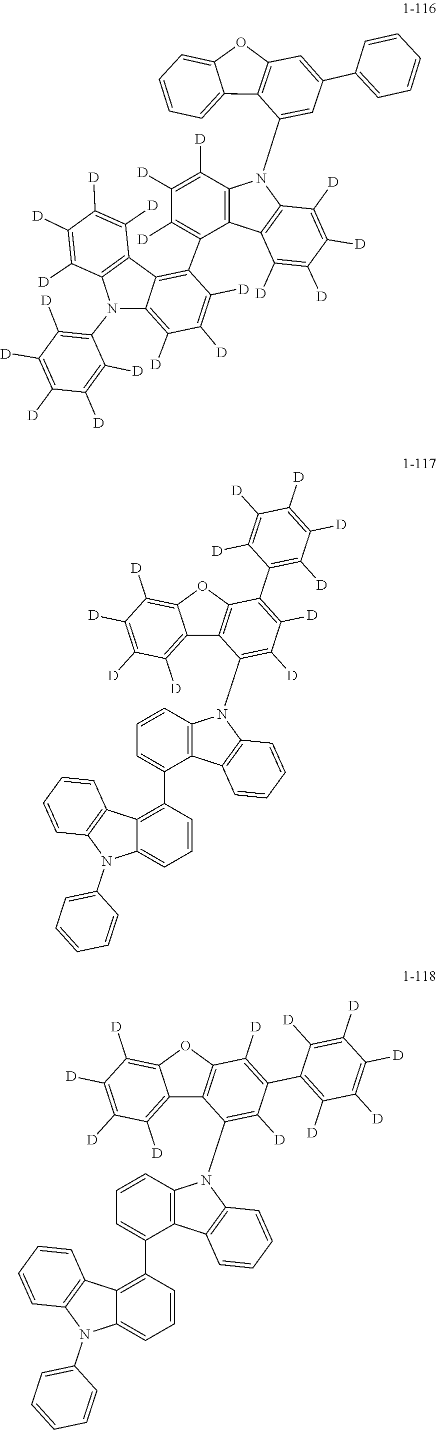 [SMILES: [2H]c1c([2H])c([2H])c(-n2c3c([2H])c([2H])c([2H])c([2H])c3c3c(-c4c([2H])c([2H])c([2H])c5c4c4c([2H])c([2H])c([2H])c([2H])c4n5-c4cc(-c5ccccc5)cc5oc6ccccc6c45)c([2H])c([2H])c([2H])c32)c([2H])c1[2H] ; [2H]c1c([2H])c([2H])c(-c2c([2H])c([2H])c(-n3c4ccccc4c4c(-c5cccc6c5c5ccccc5n6-c5ccccc5)cccc43)c3c2oc2c([2H])c([2H])c([2H])c([2H])c23)c([2H])c1[2H] ; [2H]c1c([2H])c([2H])c(-c2c([2H])c(-n3c4ccccc4c4c(-c5cccc6c5c5ccccc5n6-c5ccccc5)cccc43)c3c(oc4c([2H])c([2H])c([2H])c([2H])c43)c2[2H])c([2H])c1[2H]]

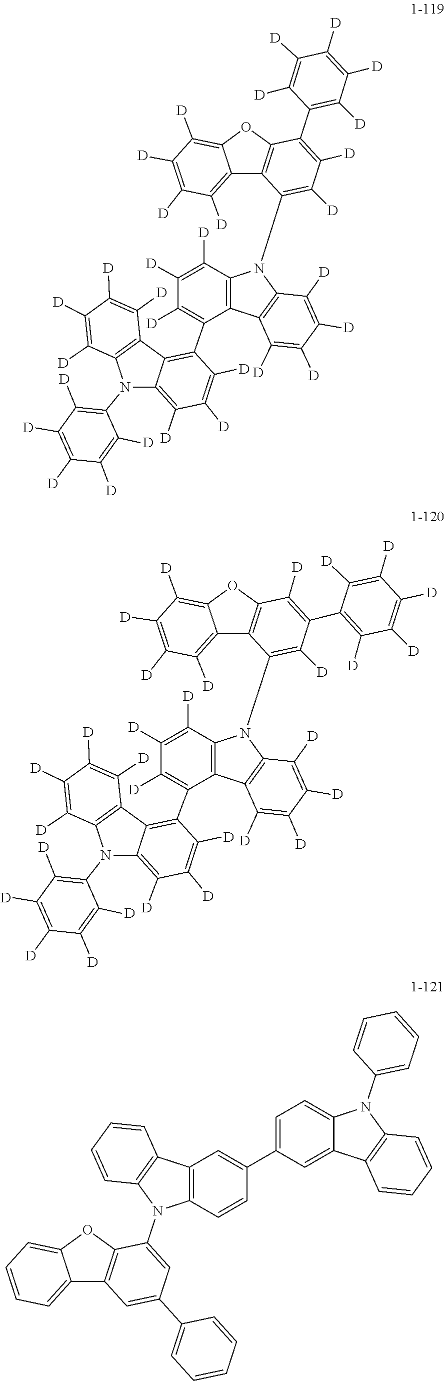

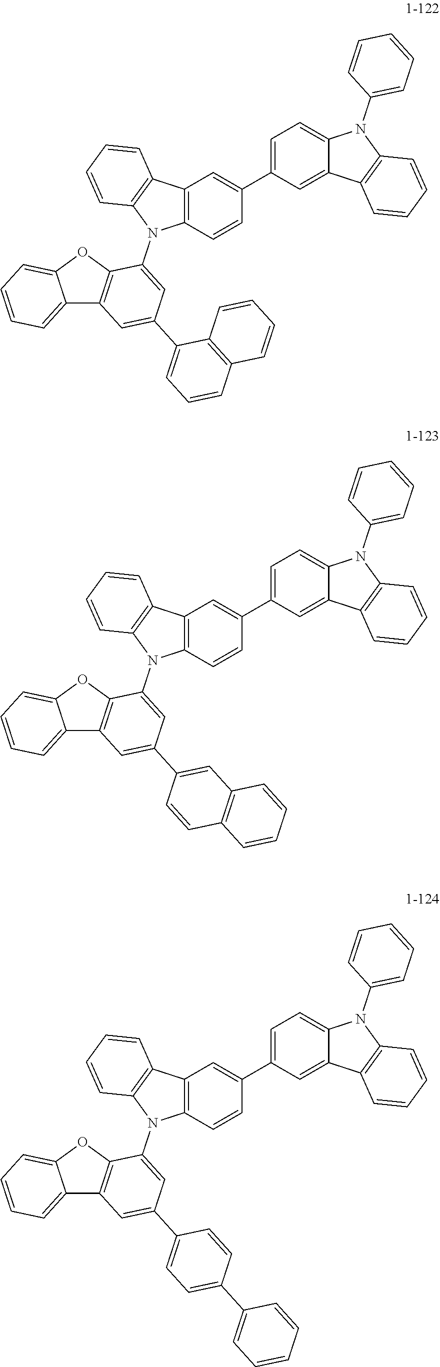 [SMILES: c1ccc(-n2c3ccccc3c3cc(-c4ccc5c(c4)c4ccccc4n5-c4cc(-c5cccc6ccccc56)cc5c4oc4ccccc45)ccc32)cc1 ; c1ccc(-n2c3ccccc3c3cc(-c4ccc5c(c4)c4ccccc4n5-c4cc(-c5ccc6ccccc6c5)cc5c4oc4ccccc45)ccc32)cc1 ; c1ccc(-c2ccc(-c3cc(-n4c5ccccc5c5cc(-c6ccc7c(c6)c6ccccc6n7-c6ccccc6)ccc54)c4oc5ccccc5c4c3)cc2)cc1]

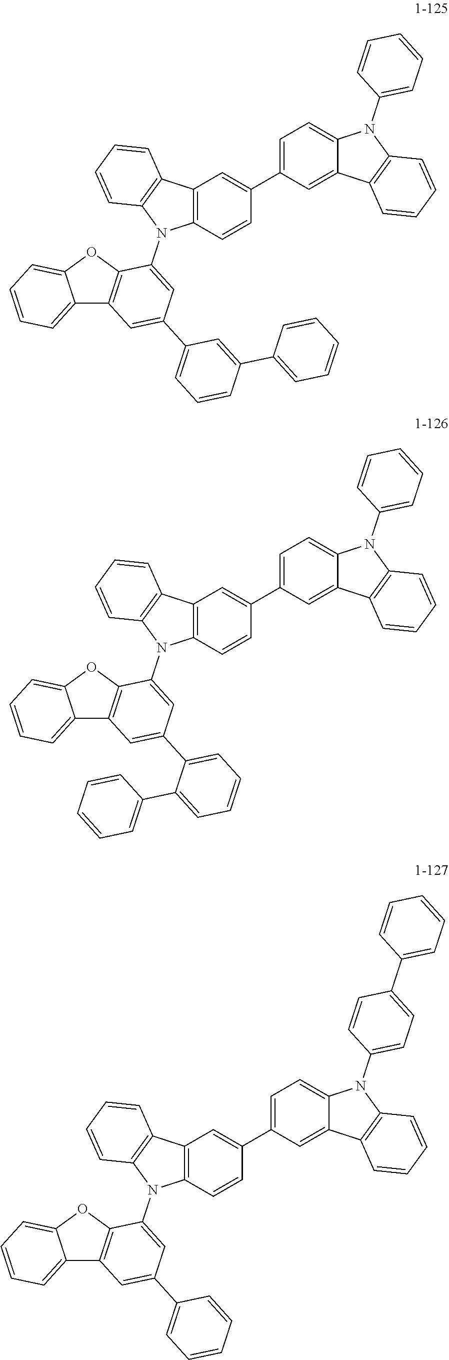 [SMILES: c1ccc(-c2cccc(-c3cc(-n4c5ccccc5c5cc(-c6ccc7c(c6)c6ccccc6n7-c6ccccc6)ccc54)c4oc5ccccc5c4c3)c2)cc1 ; c1ccc(-c2ccccc2-c2cc(-n3c4ccccc4c4cc(-c5ccc6c(c5)c5ccccc5n6-c5ccccc5)ccc43)c3oc4ccccc4c3c2)cc1 ; c1ccc(-c2ccc(-n3c4ccccc4c4cc(-c5ccc6c(c5)c5ccccc5n6-c5cc(-c6ccccc6)cc6c5oc5ccccc56)ccc43)cc2)cc1]

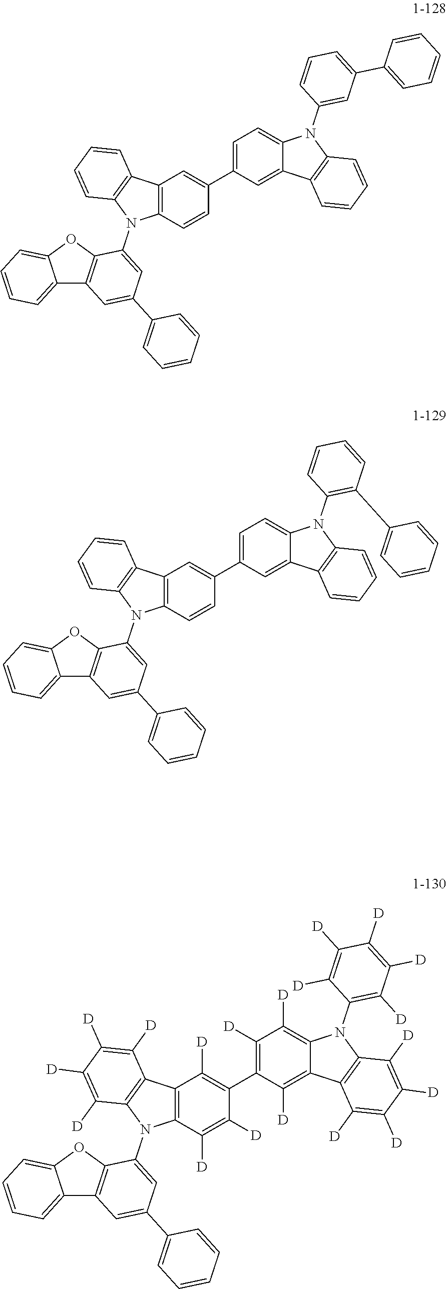 [SMILES: c1ccc(-c2cccc(-n3c4ccccc4c4cc(-c5ccc6c(c5)c5ccccc5n6-c5cc(-c6ccccc6)cc6c5oc5ccccc56)ccc43)c2)cc1 ; c1ccc(-c2cc(-n3c4ccccc4c4cc(-c5ccc6c(c5)c5ccccc5n6-c5ccccc5-c5ccccc5)ccc43)c3oc4ccccc4c3c2)cc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cc(-c5ccccc5)cc5c4oc4ccccc45)c([2H])c([2H])c32)c([2H])c1[2H]]

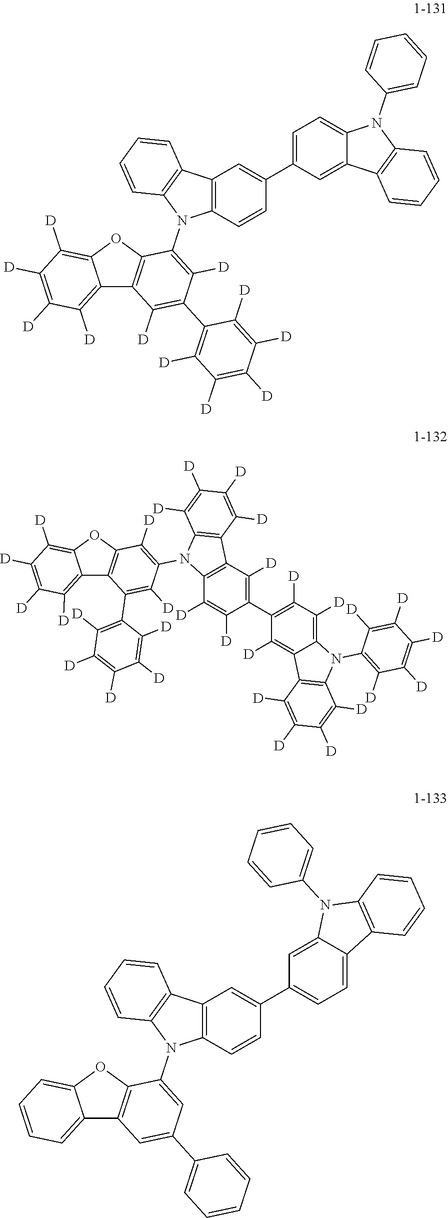 [SMILES: [2H]c1c([2H])c([2H])c(-c2c([2H])c(-n3c4ccccc4c4cc(-c5ccc6c(c5)c5ccccc5n6-c5ccccc5)ccc43)c3oc4c([2H])c([2H])c([2H])c([2H])c4c3c2[2H])c([2H])c1[2H] ; [2H]c1c([2H])c([2H])c(-c2c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c([2H])c5[2H])c([2H])c([2H])c43)c([2H])c3oc4c([2H])c([2H])c([2H])c([2H])c4c23)c([2H])c1[2H] ; c1ccc(-c2cc(-n3c4ccccc4c4cc(-c5ccc6c7ccccc7n(-c7ccccc7)c6c5)ccc43)c3oc4ccccc4c3c2)cc1]

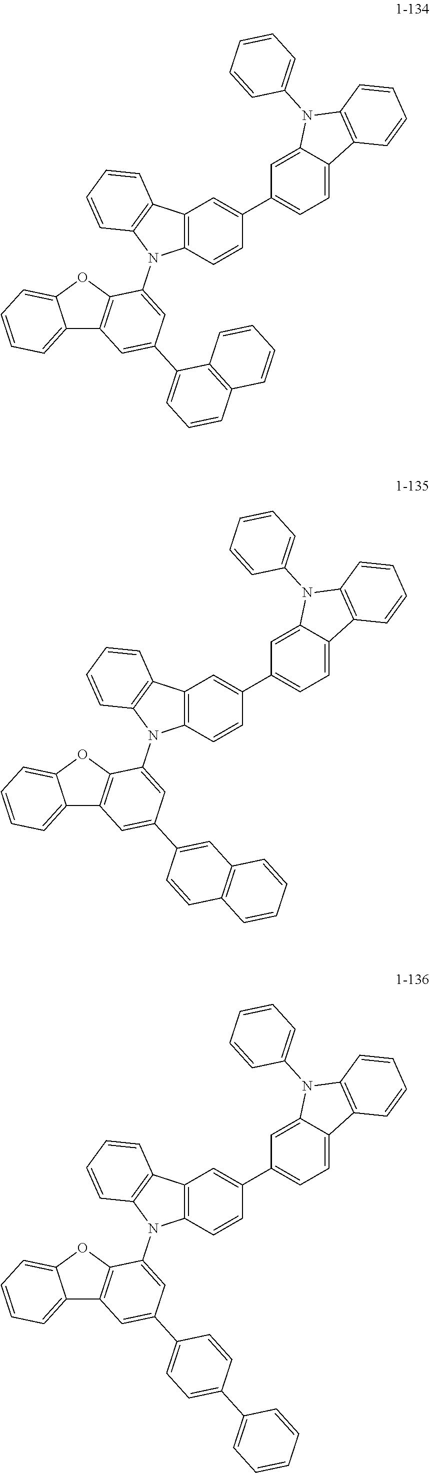 [SMILES: c1ccc(-n2c3ccccc3c3ccc(-c4ccc5c(c4)c4ccccc4n5-c4cc(-c5cccc6ccccc56)cc5c4oc4ccccc45)cc32)cc1 ; c1ccc(-n2c3ccccc3c3ccc(-c4ccc5c(c4)c4ccccc4n5-c4cc(-c5ccc6ccccc6c5)cc5c4oc4ccccc45)cc32)cc1 ; c1ccc(-c2ccc(-c3cc(-n4c5ccccc5c5cc(-c6ccc7c8ccccc8n(-c8ccccc8)c7c6)ccc54)c4oc5ccccc5c4c3)cc2)cc1]

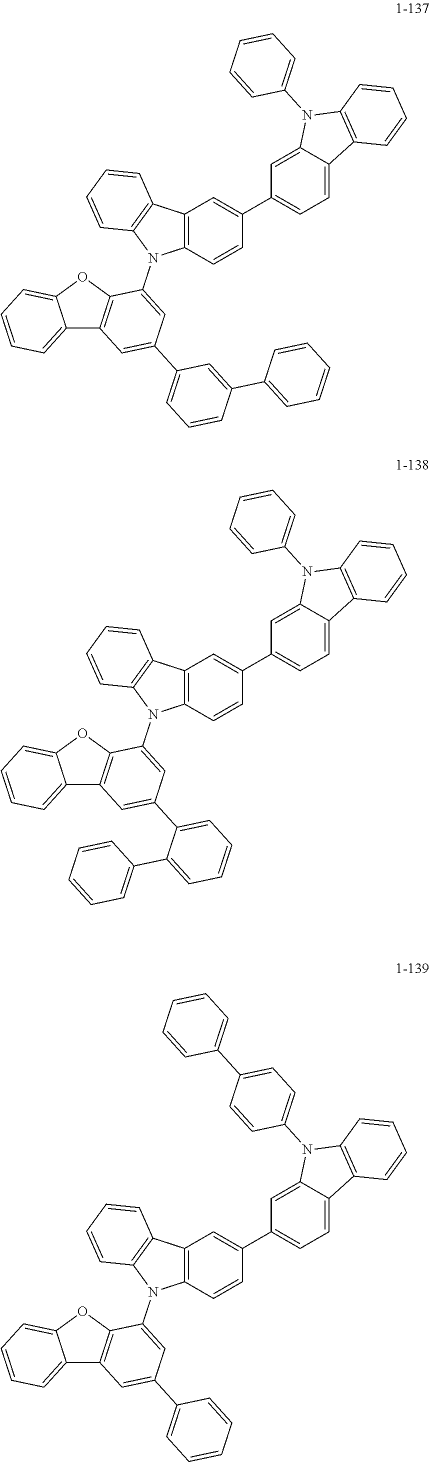 [SMILES: c1ccc(-c2cccc(-c3cc(-n4c5ccccc5c5cc(-c6ccc7c8ccccc8n(-c8ccccc8)c7c6)ccc54)c4oc5ccccc5c4c3)c2)cc1 ; c1ccc(-c2ccccc2-c2cc(-n3c4ccccc4c4cc(-c5ccc6c7ccccc7n(-c7ccccc7)c6c5)ccc43)c3oc4ccccc4c3c2)cc1 ; c1ccc(-c2ccc(-n3c4ccccc4c4ccc(-c5ccc6c(c5)c5ccccc5n6-c5cc(-c6ccccc6)cc6c5oc5ccccc56)cc43)cc2)cc1]

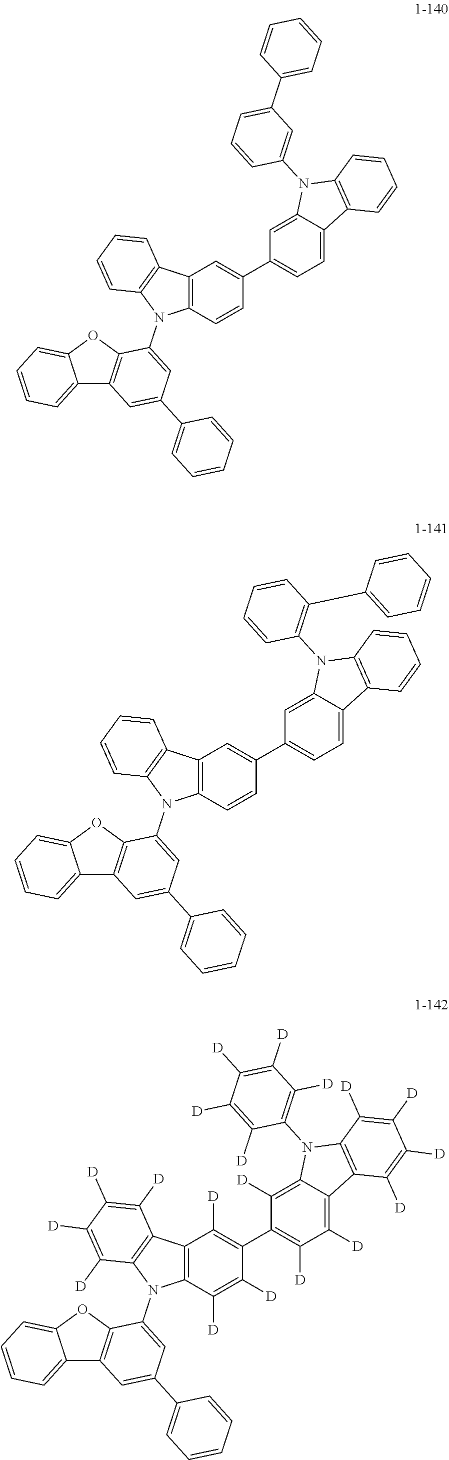 [SMILES: c1ccc(-c2cccc(-n3c4ccccc4c4ccc(-c5ccc6c(c5)c5ccccc5n6-c5cc(-c6ccccc6)cc6c5oc5ccccc56)cc43)c2)cc1 ; c1ccc(-c2cc(-n3c4ccccc4c4cc(-c5ccc6c7ccccc7n(-c7ccccc7-c7ccccc7)c6c5)ccc43)c3oc4ccccc4c3c2)cc1 ; [2H]c1c([2H])c([2H])c(-n2c3c([2H])c([2H])c([2H])c([2H])c3c3c([2H])c([2H])c(-c4c([2H])c([2H])c5c(c4[2H])c4c([2H])c([2H])c([2H])c([2H])c4n5-c4cc(-c5ccccc5)cc5c4oc4ccccc45)c([2H])c32)c([2H])c1[2H]]

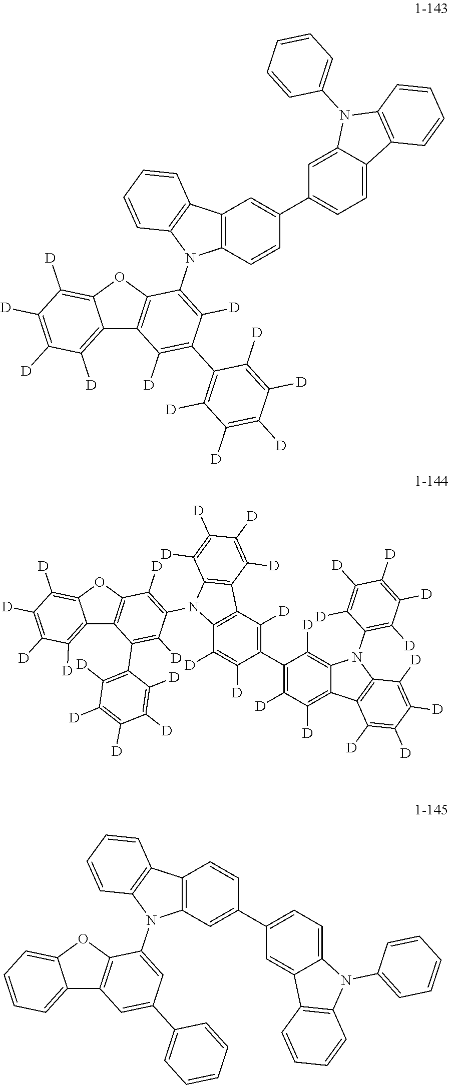 [SMILES: [2H]c1c([2H])c([2H])c(-c2c([2H])c(-n3c4ccccc4c4cc(-c5ccc6c7ccccc7n(-c7ccccc7)c6c5)ccc43)c3oc4c([2H])c([2H])c([2H])c([2H])c4c3c2[2H])c([2H])c1[2H] ; [2H]c1c([2H])c([2H])c(-c2c([2H])c(-n3c4c([2H])c([2H])c([2H])c([2H])c4c4c([2H])c(-c5c([2H])c([2H])c6c7c([2H])c([2H])c([2H])c([2H])c7n(-c7c([2H])c([2H])c([2H])c([2H])c7[2H])c6c5[2H])c([2H])c([2H])c43)c([2H])c3oc4c([2H])c([2H])c([2H])c([2H])c4c23)c([2H])c1[2H] ; c1ccc(-c2cc(-n3c4ccccc4c4ccc(-c5ccc6c(c5)c5ccccc5n6-c5ccccc5)cc43)c3oc4ccccc4c3c2)cc1]

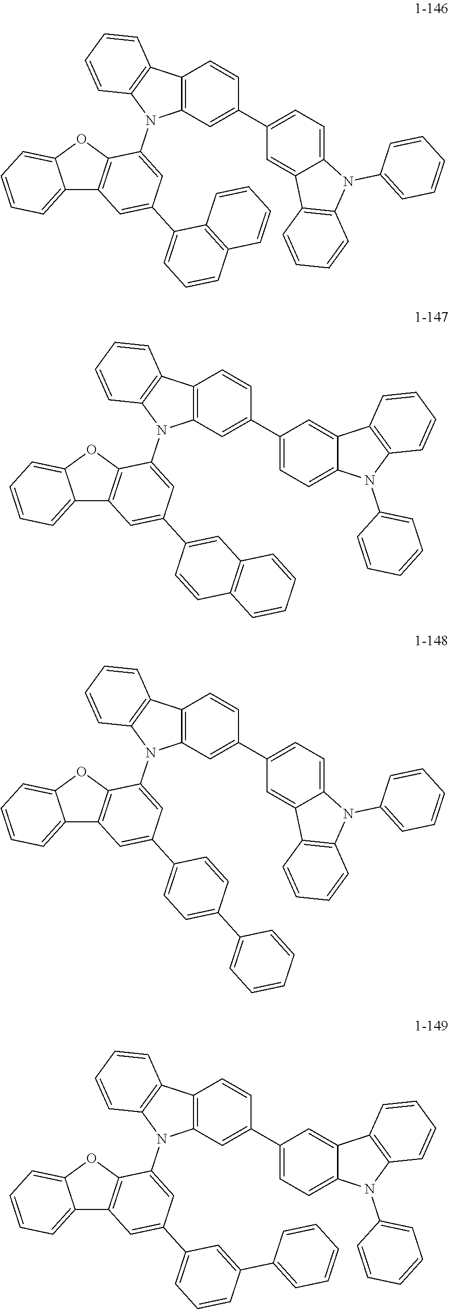 [SMILES: c1ccc(-n2c3ccccc3c3cc(-c4ccc5c6ccccc6n(-c6cc(-c7cccc8ccccc78)cc7c6oc6ccccc67)c5c4)ccc32)cc1 ; c1ccc(-n2c3ccccc3c3cc(-c4ccc5c6ccccc6n(-c6cc(-c7ccc8ccccc8c7)cc7c6oc6ccccc67)c5c4)ccc32)cc1 ; c1ccc(-c2ccc(-c3cc(-n4c5ccccc5c5ccc(-c6ccc7c(c6)c6ccccc6n7-c6ccccc6)cc54)c4oc5ccccc5c4c3)cc2)cc1 ; c1ccc(-c2cccc(-c3cc(-n4c5ccccc5c5ccc(-c6ccc7c(c6)c6ccccc6n7-c6ccccc6)cc54)c4oc5ccccc5c4c3)c2)cc1]

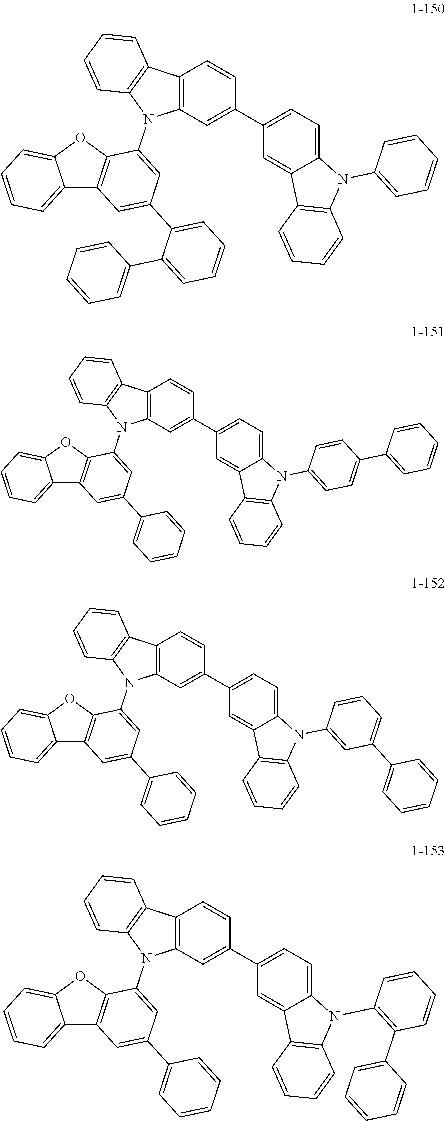 [SMILES: c1ccc(-c2ccccc2-c2cc(-n3c4ccccc4c4ccc(-c5ccc6c(c5)c5ccccc5n6-c5ccccc5)cc43)c3oc4ccccc4c3c2)cc1 ; c1ccc(-c2ccc(-n3c4ccccc4c4cc(-c5ccc6c7ccccc7n(-c7cc(-c8ccccc8)cc8c7oc7ccccc78)c6c5)ccc43)cc2)cc1 ; c1ccc(-c2cccc(-n3c4ccccc4c4cc(-c5ccc6c7ccccc7n(-c7cc(-c8ccccc8)cc8c7oc7ccccc78)c6c5)ccc43)c2)cc1 ; c1ccc(-c2cc(-n3c4ccccc4c4ccc(-c5ccc6c(c5)c5ccccc5n6-c5ccccc5-c5ccccc5)cc43)c3oc4ccccc4c3c2)cc1]

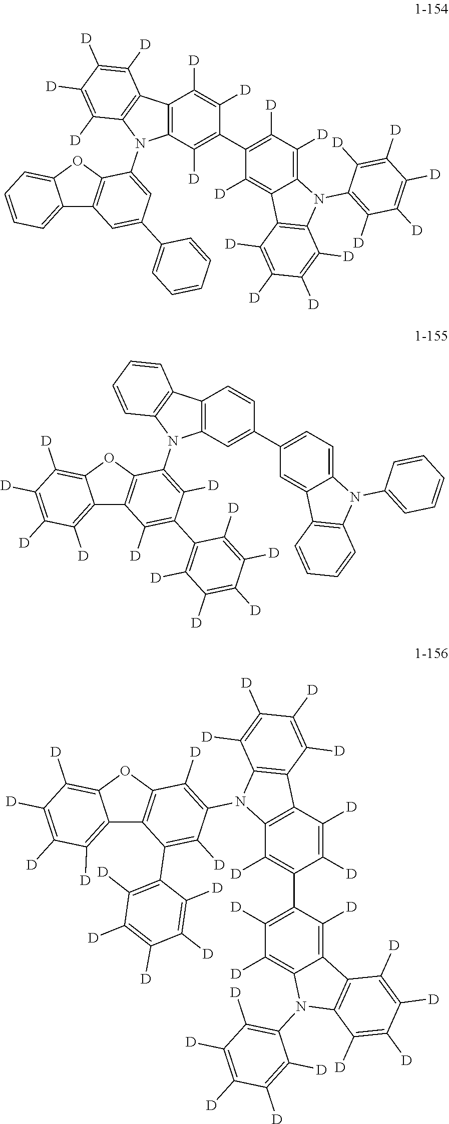

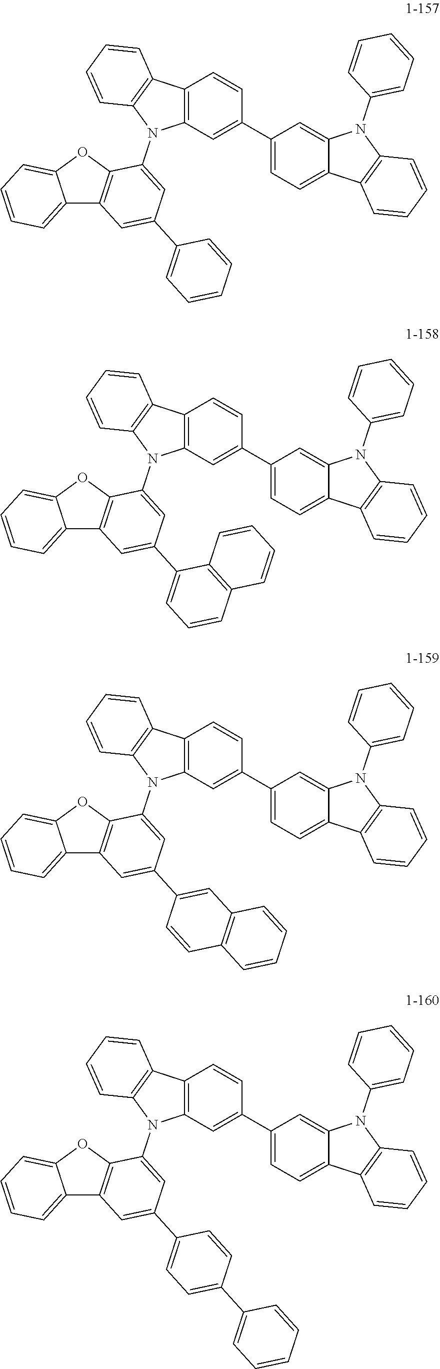 [SMILES: c1ccc(-c2cc(-n3c4ccccc4c4ccc(-c5ccc6c7ccccc7n(-c7ccccc7)c6c5)cc43)c3oc4ccccc4c3c2)cc1 ; c1ccc(-n2c3ccccc3c3ccc(-c4ccc5c6ccccc6n(-c6cc(-c7cccc8ccccc78)cc7c6oc6ccccc67)c5c4)cc32)cc1 ; c1ccc(-n2c3ccccc3c3ccc(-c4ccc5c6ccccc6n(-c6cc(-c7ccc8ccccc8c7)cc7c6oc6ccccc67)c5c4)cc32)cc1 ; c1ccc(-c2ccc(-c3cc(-n4c5ccccc5c5ccc(-c6ccc7c8ccccc8n(-c8ccccc8)c7c6)cc54)c4oc5ccccc5c4c3)cc2)cc1]

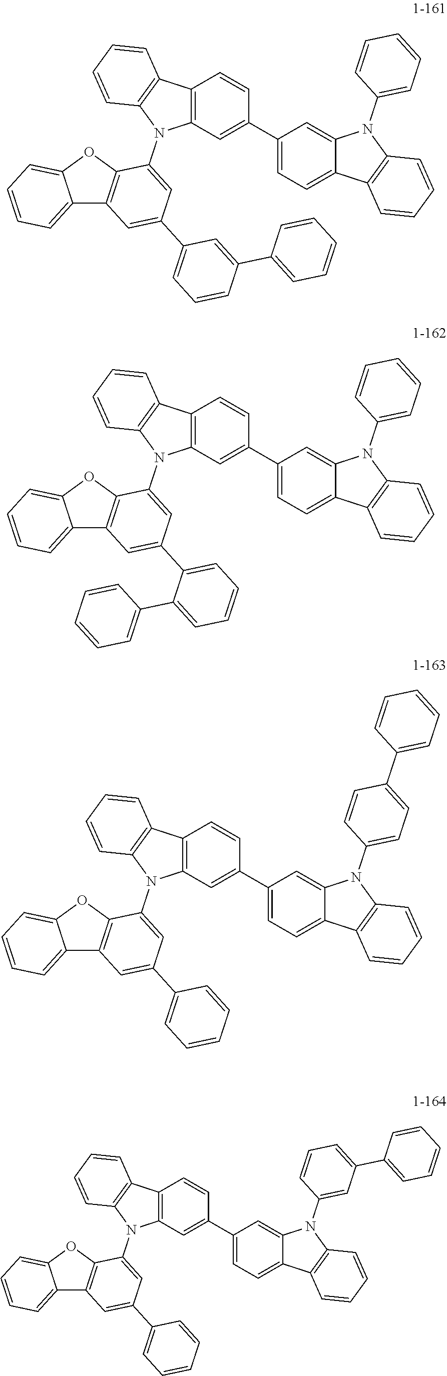 [SMILES: c1ccc(-c2cccc(-c3cc(-n4c5ccccc5c5ccc(-c6ccc7c8ccccc8n(-c8ccccc8)c7c6)cc54)c4oc5ccccc5c4c3)c2)cc1 ; c1ccc(-c2ccccc2-c2cc(-n3c4ccccc4c4ccc(-c5ccc6c7ccccc7n(-c7ccccc7)c6c5)cc43)c3oc4ccccc4c3c2)cc1 ; c1ccc(-c2ccc(-n3c4ccccc4c4ccc(-c5ccc6c7ccccc7n(-c7cc(-c8ccccc8)cc8c7oc7ccccc78)c6c5)cc43)cc2)cc1 ; c1ccc(-c2cccc(-n3c4ccccc4c4ccc(-c5ccc6c7ccccc7n(-c7cc(-c8ccccc8)cc8c7oc7ccccc78)c6c5)cc43)c2)cc1]

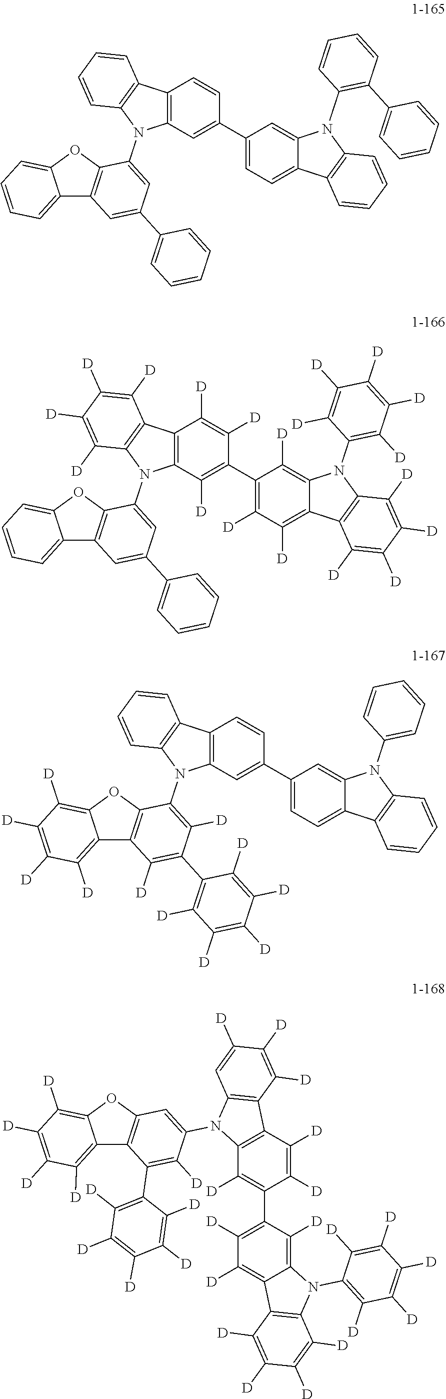

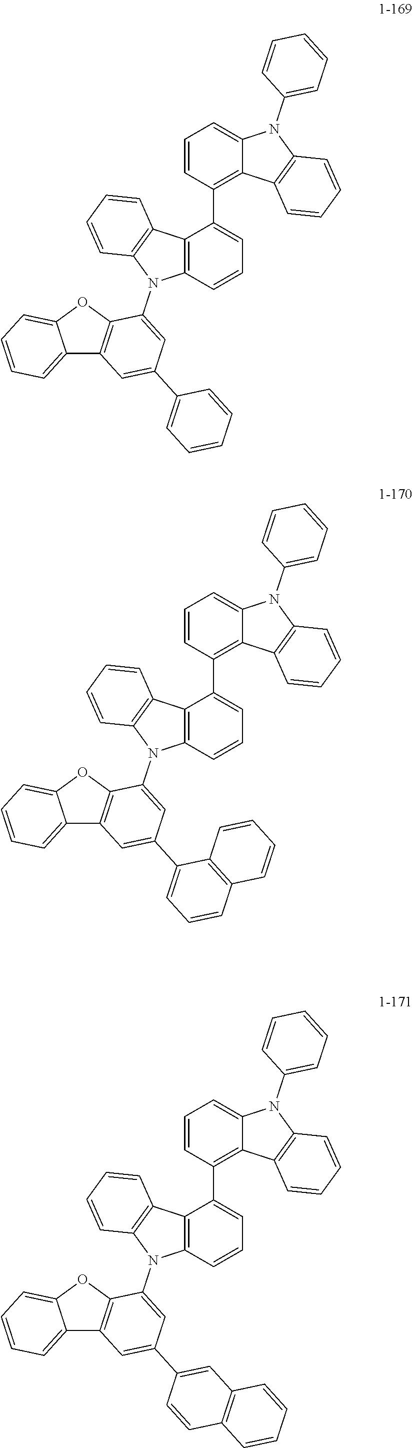 [SMILES: c1ccc(-c2cc(-n3c4ccccc4c4c(-c5cccc6c5c5ccccc5n6-c5ccccc5)cccc43)c3oc4ccccc4c3c2)cc1 ; c1ccc(-n2c3ccccc3c3c(-c4cccc5c4c4ccccc4n5-c4cc(-c5cccc6ccccc56)cc5c4oc4ccccc45)cccc32)cc1 ; c1ccc(-n2c3ccccc3c3c(-c4cccc5c4c4ccccc4n5-c4cc(-c5ccc6ccccc6c5)cc5c4oc4ccccc45)cccc32)cc1]

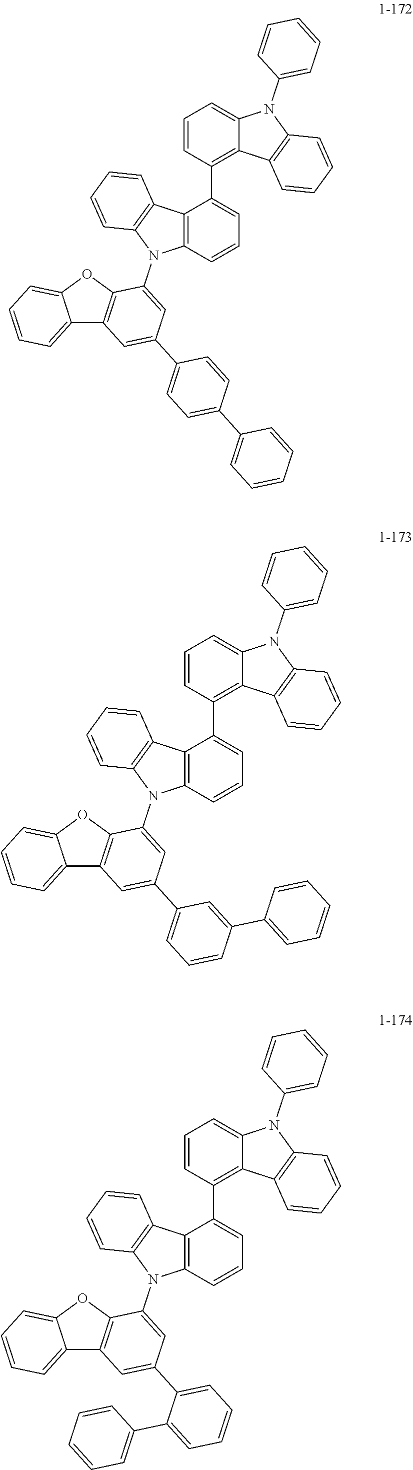 [SMILES: c1ccc(-c2ccc(-c3cc(-n4c5ccccc5c5c(-c6cccc7c6c6ccccc6n7-c6ccccc6)cccc54)c4oc5ccccc5c4c3)cc2)cc1 ; c1ccc(-c2cccc(-c3cc(-n4c5ccccc5c5c(-c6cccc7c6c6ccccc6n7-c6ccccc6)cccc54)c4oc5ccccc5c4c3)c2)cc1 ; c1ccc(-c2ccccc2-c2cc(-n3c4ccccc4c4c(-c5cccc6c5c5ccccc5n6-c5ccccc5)cccc43)c3oc4ccccc4c3c2)cc1]

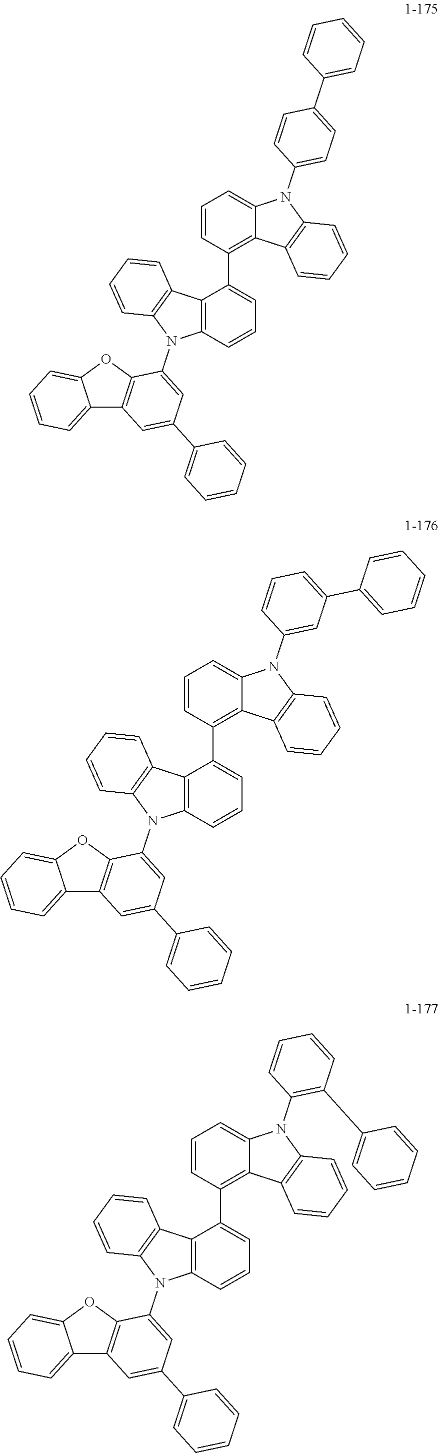 [SMILES: c1ccc(-c2ccc(-n3c4ccccc4c4c(-c5cccc6c5c5ccccc5n6-c5cc(-c6ccccc6)cc6c5oc5ccccc56)cccc43)cc2)cc1 ; c1ccc(-c2cccc(-n3c4ccccc4c4c(-c5cccc6c5c5ccccc5n6-c5cc(-c6ccccc6)cc6c5oc5ccccc56)cccc43)c2)cc1 ; c1ccc(-c2cc(-n3c4ccccc4c4c(-c5cccc6c5c5ccccc5n6-c5ccccc5-c5ccccc5)cccc43)c3oc4ccccc4c3c2)cc1]

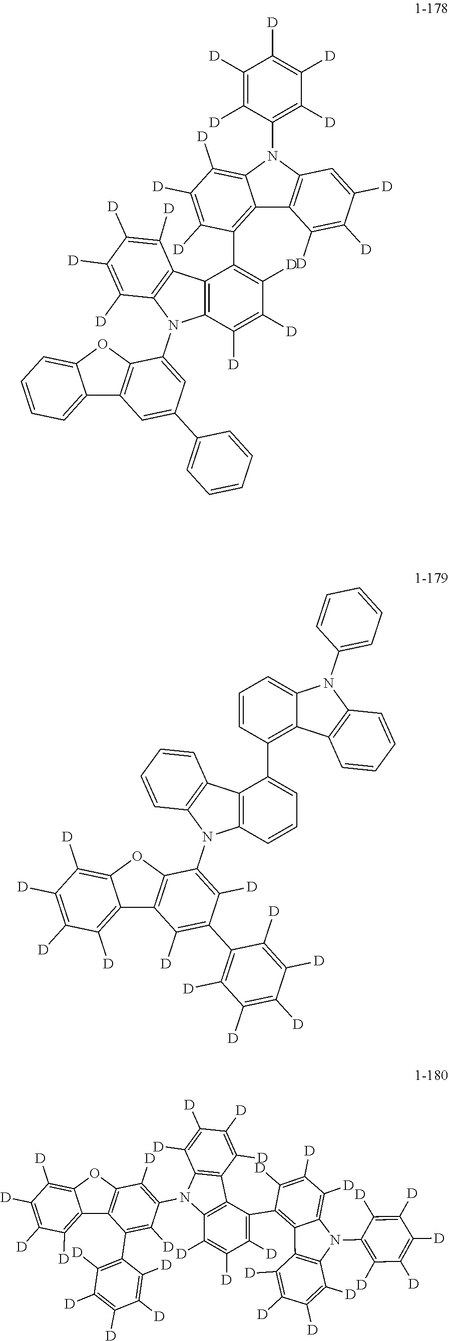

8. An organic light emitting device comprising:
a first electrode;
a second electrode; and an organic material layer having one or more layers provided between the first electrode and the second electrode,
wherein one or more layers of the organic material layer comprise the heterocyclic compound according to claim 1.

9. The organic light emitting device of claim 8, wherein the organic material layer comprises a light emitting layer, and the light emitting layer comprises the heterocyclic compound.

10. The organic light emitting device of claim 8, further comprising one or two or more layers selected from the group consisting of a light emitting layer, a hole injection layer, a hole transport layer, an electron injection layer, an electron transport layer, a hole auxiliary layer, and a hole blocking layer.

11. The organic light emitting device of claim 8, wherein one or more layers of the organic material layer comprise both the heterocyclic compound represented by Chemical Formula 1 and a heterocyclic compound represented by the following Chemical Formula 2: 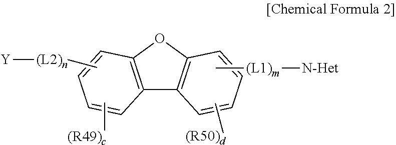
in Chemical Formula 2,
N-Het is a monocyclic or polycyclic heterocyclic group which is substituted or unsubstituted and comprises one or more N's,
L1 and L2 are the same as or different from each other, and are each independently a direct bond; a substituted or unsubstituted arylene group having 6 to 60 carbon atoms; or a substituted or unsubstituted heteroarylene group having 2 to 60 carbon atoms, m and n are each an integer from 0 to 3, when m is 2 or higher, L1's are the same as or different from each other, and when n is 2 or higher, L2's are the same as or different from each other,
R49 and R50 are the same as or different from each other, and are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or a substituted or unsubstituted hetero ring, c and d are each an integer from 0 to 3, when c is 2 or higher, R49's are the same as or different from each other, and when d is 2 or higher, R50's are the same as or different from each other, and
Y is a hole transporting group; or a substituted or unsubstituted aryl group having 6 to 60 carbon atoms.

12. The organic light emitting device of claim 11, wherein the N-Het, L1, L2, R49 and R50 are substituted with one or more deuteriums, and the content of the substituted deuterium is 0% to 100%.

13. The organic light emitting device of claim 11, wherein at least one of the compound represented by Chemical Formula 1 and the compound represented by Chemical Formula 2 has a deuterium content of more than 0% and 100% or less.

14. The organic light emitting device of claim 11, wherein Chemical Formula 2 is represented by any one of the following compounds:

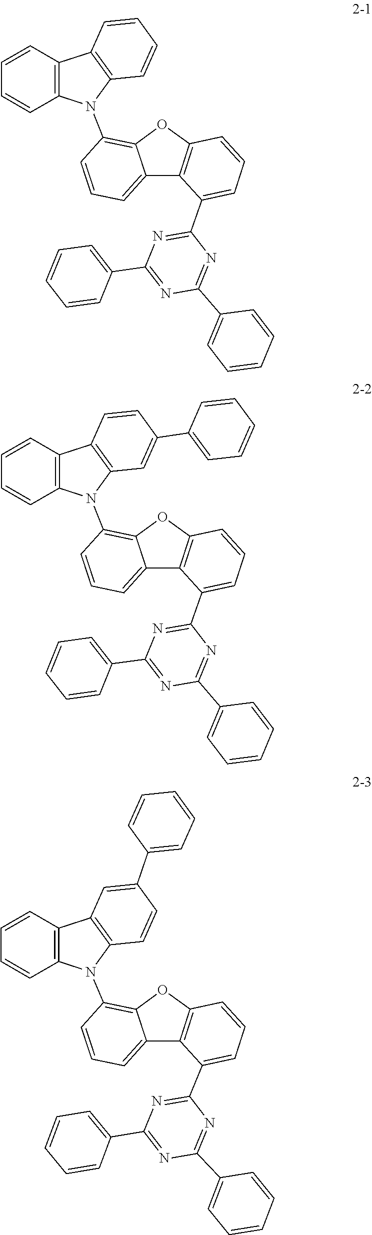

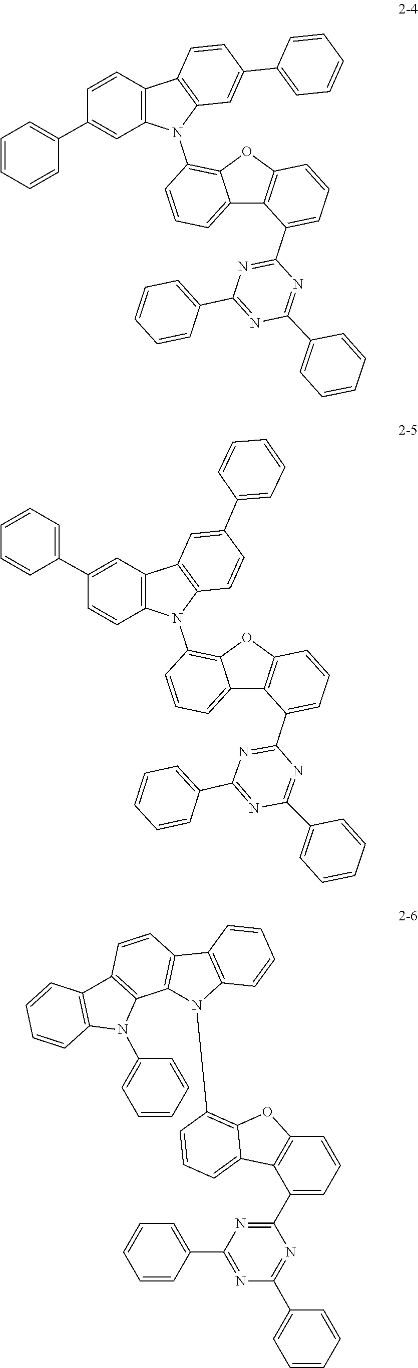 [SMILES: c1ccc(-c2ccc3c4ccc(-c5ccccc5)cc4n(-c4cccc5c4oc4cccc(-c6nc(-c7ccccc7)nc(-c7ccccc7)n6)c45)c3c2)cc1 ; c1ccc(-c2ccc3c(c2)c2cc(-c4ccccc4)ccc2n3-c2cccc3c2oc2cccc(-c4nc(-c5ccccc5)nc(-c5ccccc5)n4)c23)cc1 ; c1ccc(-c2nc(-c3ccccc3)nc(-c3cccc4oc5c(-n6c7ccccc7c7ccc8c9ccccc9n(-c9ccccc9)c8c76)cccc5c34)n2)cc1]

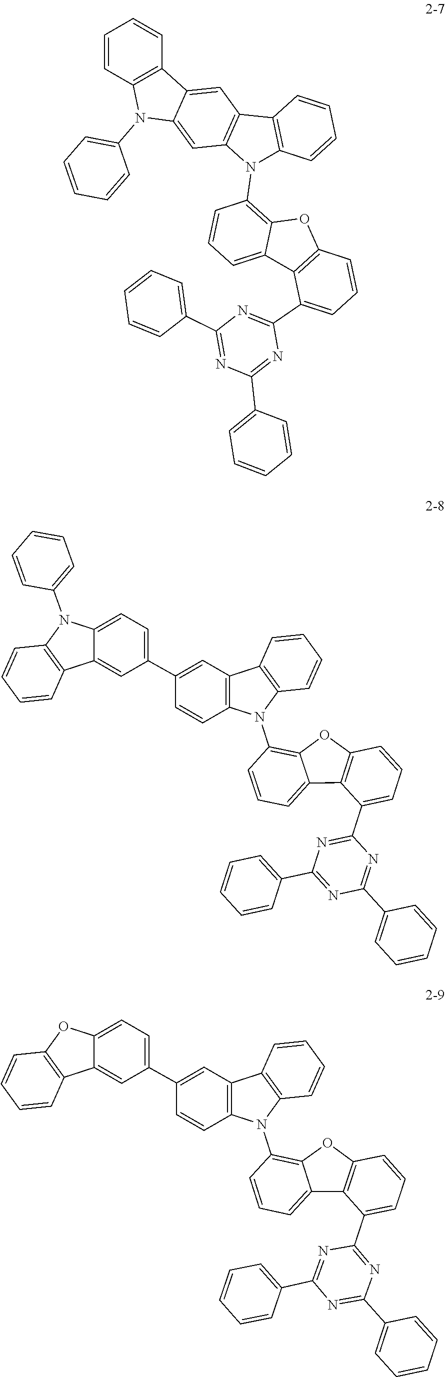 [SMILES: c1ccc(-c2nc(-c3ccccc3)nc(-c3cccc4oc5c(-n6c7ccccc7c7cc8c9ccccc9n(-c9ccccc9)c8cc76)cccc5c34)n2)cc1 ; c1ccc(-c2nc(-c3ccccc3)nc(-c3cccc4oc5c(-n6c7ccccc7c7cc(-c8ccc9c(c8)c8ccccc8n9-c8ccccc8)ccc76)cccc5c34)n2)cc1 ; c1ccc(-c2nc(-c3ccccc3)nc(-c3cccc4oc5c(-n6c7ccccc7c7cc(-c8ccc9oc%10ccccc%10c9c8)ccc76)cccc5c34)n2)cc1]

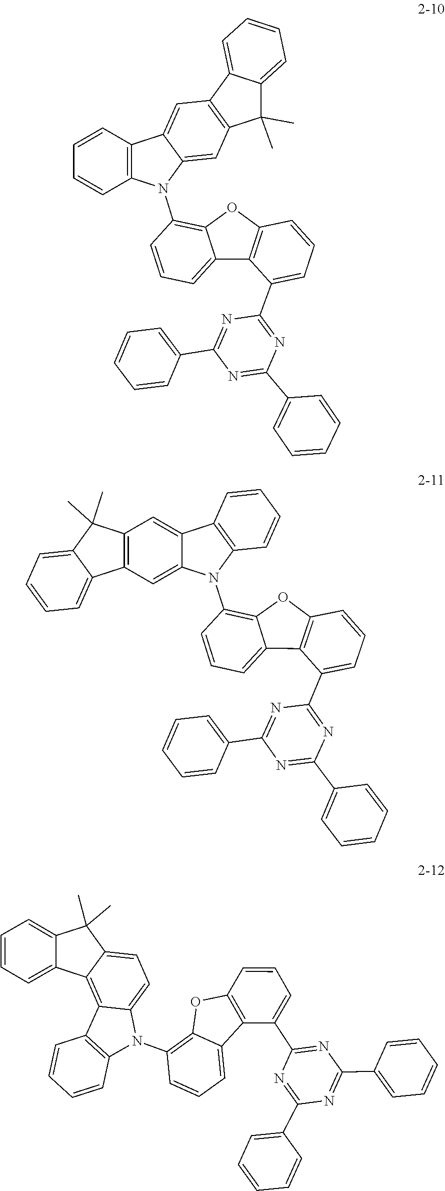

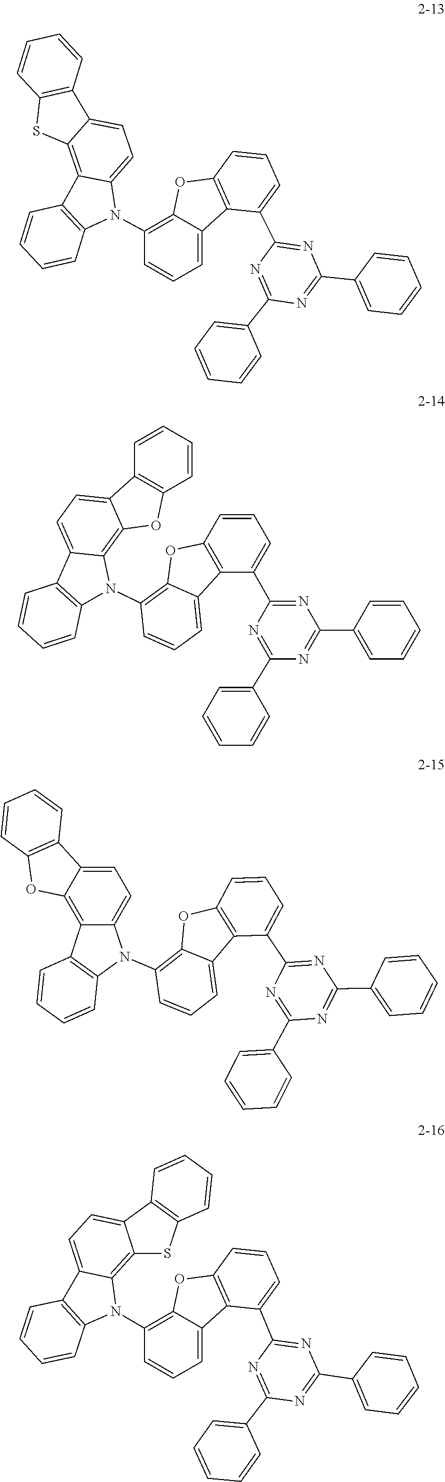 [SMILES: c1ccc(-c2nc(-c3ccccc3)nc(-c3cccc4oc5c(-n6c7ccccc7c7c8sc9ccccc9c8ccc76)cccc5c34)n2)cc1 ; c1ccc(-c2nc(-c3ccccc3)nc(-c3cccc4oc5c(-n6c7ccccc7c7ccc8c9ccccc9oc8c76)cccc5c34)n2)cc1 ; c1ccc(-c2nc(-c3ccccc3)nc(-c3cccc4oc5c(-n6c7ccccc7c7c8oc9ccccc9c8ccc76)cccc5c34)n2)cc1 ; c1ccc(-c2nc(-c3ccccc3)nc(-c3cccc4oc5c(-n6c7ccccc7c7ccc8c9ccccc9sc8c76)cccc5c34)n2)cc1]

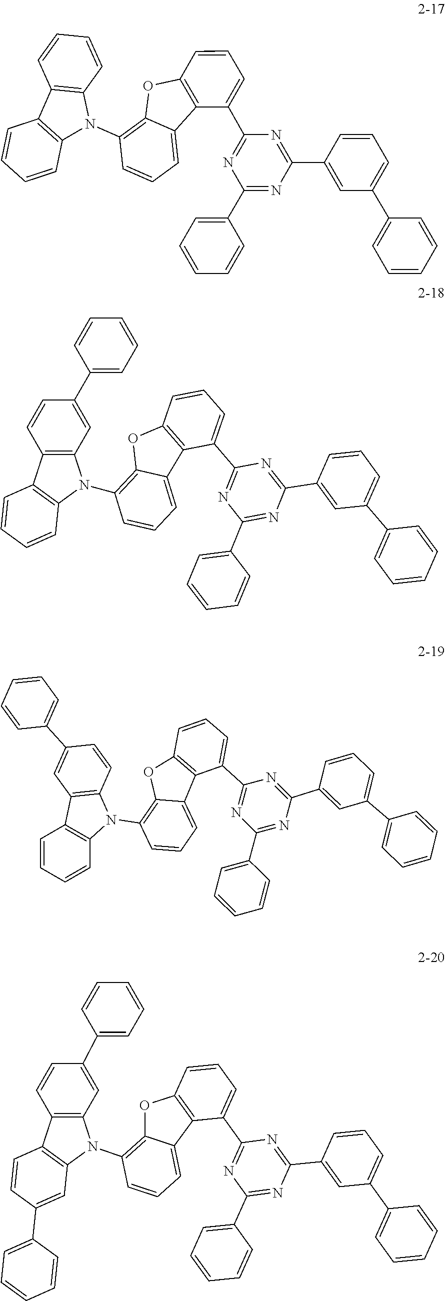 [SMILES: c1ccc(-c2cccc(-c3nc(-c4ccccc4)nc(-c4cccc5oc6c(-n7c8ccccc8c8ccccc87)cccc6c45)n3)c2)cc1 ; c1ccc(-c2cccc(-c3nc(-c4ccccc4)nc(-c4cccc5oc6c(-n7c8ccccc8c8ccc(-c9ccccc9)cc87)cccc6c45)n3)c2)cc1 ; c1ccc(-c2cccc(-c3nc(-c4ccccc4)nc(-c4cccc5oc6c(-n7c8ccccc8c8cc(-c9ccccc9)ccc87)cccc6c45)n3)c2)cc1 ; c1ccc(-c2cccc(-c3nc(-c4ccccc4)nc(-c4cccc5oc6c(-n7c8cc(-c9ccccc9)ccc8c8ccc(-c9ccccc9)cc87)cccc6c45)n3)c2)cc1]

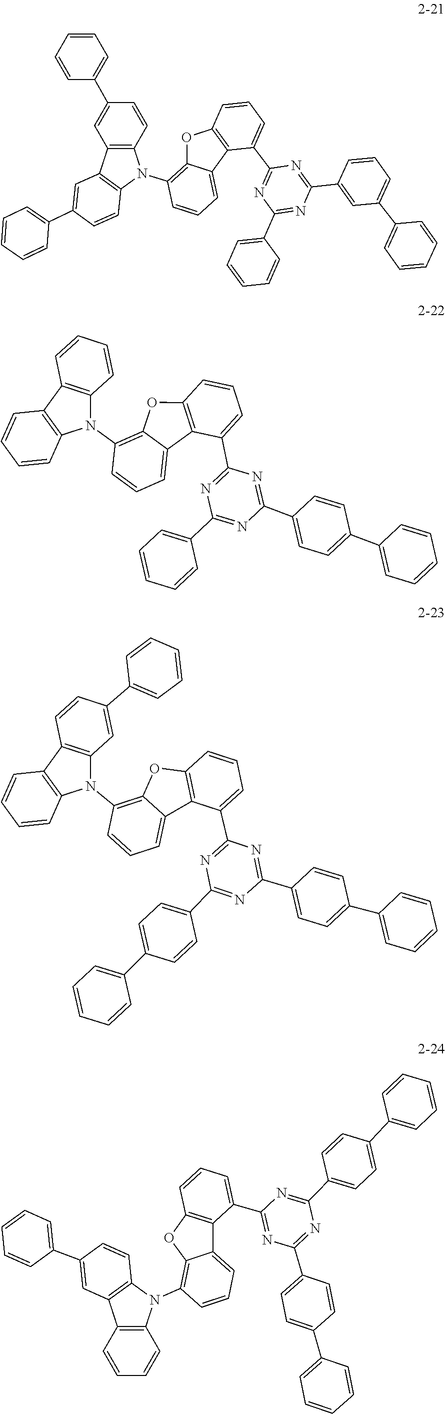 [SMILES: c1ccc(-c2cccc(-c3nc(-c4ccccc4)nc(-c4cccc5oc6c(-n7c8ccc(-c9ccccc9)cc8c8cc(-c9ccccc9)ccc87)cccc6c45)n3)c2)cc1 ; c1ccc(-c2ccc(-c3nc(-c4ccccc4)nc(-c4cccc5oc6c(-n7c8ccccc8c8ccccc87)cccc6c45)n3)cc2)cc1 ; c1ccc(-c2ccc(-c3nc(-c4ccc(-c5ccccc5)cc4)nc(-c4cccc5oc6c(-n7c8ccccc8c8ccc(-c9ccccc9)cc87)cccc6c45)n3)cc2)cc1 ; c1ccc(-c2ccc(-c3nc(-c4ccc(-c5ccccc5)cc4)nc(-c4cccc5oc6c(-n7c8ccccc8c8cc(-c9ccccc9)ccc87)cccc6c45)n3)cc2)cc1]

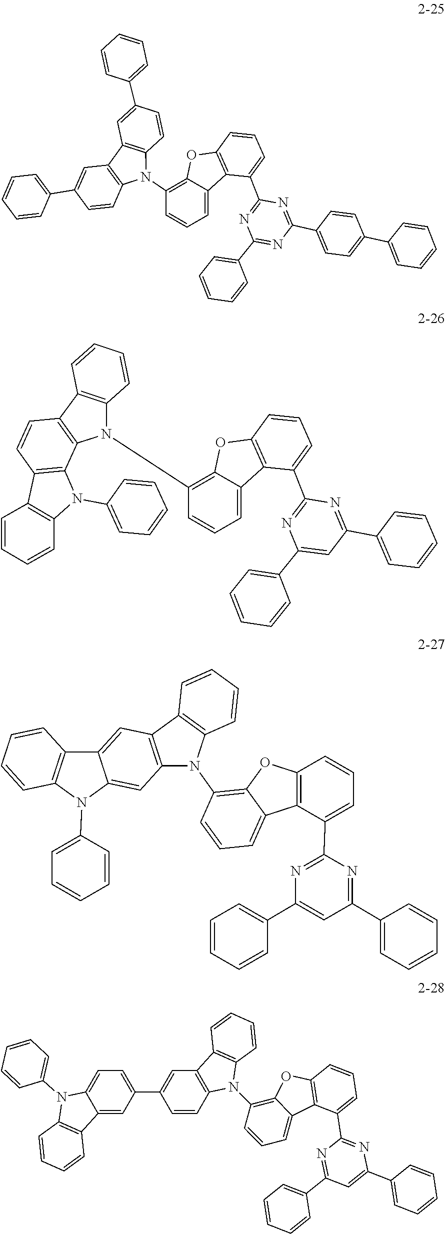 [SMILES: c1ccc(-c2ccc(-c3nc(-c4ccccc4)nc(-c4cccc5oc6c(-n7c8ccc(-c9ccccc9)cc8c8cc(-c9ccccc9)ccc87)cccc6c45)n3)cc2)cc1 ; c1ccc(-c2cc(-c3ccccc3)nc(-c3cccc4oc5c(-n6c7ccccc7c7ccc8c9ccccc9n(-c9ccccc9)c8c76)cccc5c34)n2)cc1 ; c1ccc(-c2cc(-c3ccccc3)nc(-c3cccc4oc5c(-n6c7ccccc7c7cc8c9ccccc9n(-c9ccccc9)c8cc76)cccc5c34)n2)cc1 ; c1ccc(-c2cc(-c3ccccc3)nc(-c3cccc4oc5c(-n6c7ccccc7c7cc(-c8ccc9c(c8)c8ccccc8n9-c8ccccc8)ccc76)cccc5c34)n2)cc1]

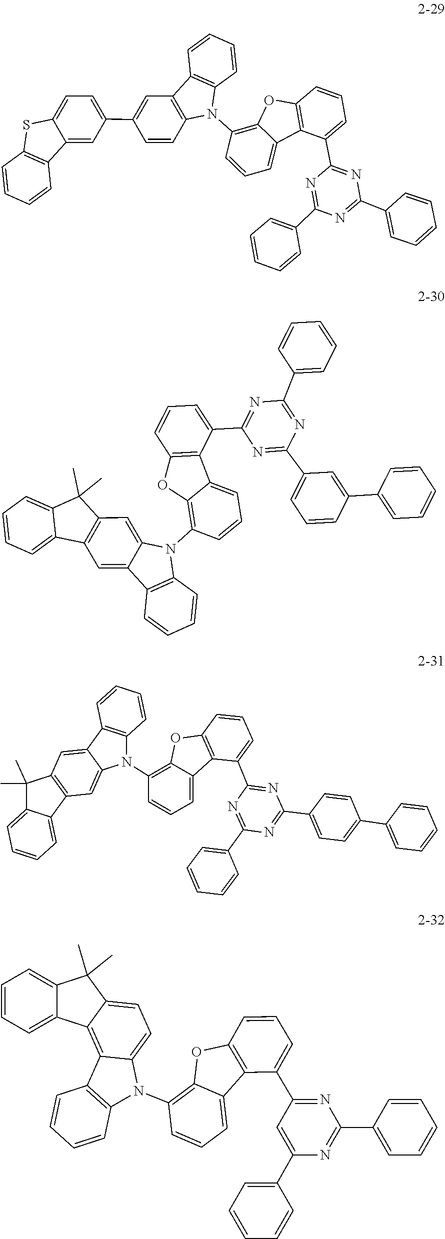 [SMILES: c1ccc(-c2nc(-c3ccccc3)nc(-c3cccc4oc5c(-n6c7ccccc7c7cc(-c8ccc9sc%10ccccc%10c9c8)ccc76)cccc5c34)n2)cc1 ; CC1(C)c2ccccc2-c2cc3c4ccccc4n(-c4cccc5c4oc4cccc(-c6nc(-c7ccccc7)nc(-c7cccc(-c8ccccc8)c7)n6)c45)c3cc21 ; CC1(C)c2ccccc2-c2cc3c(cc21)c1ccccc1n3-c1cccc2c1oc1cccc(-c3nc(-c4ccccc4)nc(-c4ccc(-c5ccccc5)cc4)n3)c12 ; CC1(C)c2ccccc2-c2c1ccc1c2c2ccccc2n1-c1cccc2c1oc1cccc(-c3cc(-c4ccccc4)nc(-c4ccccc4)n3)c12]

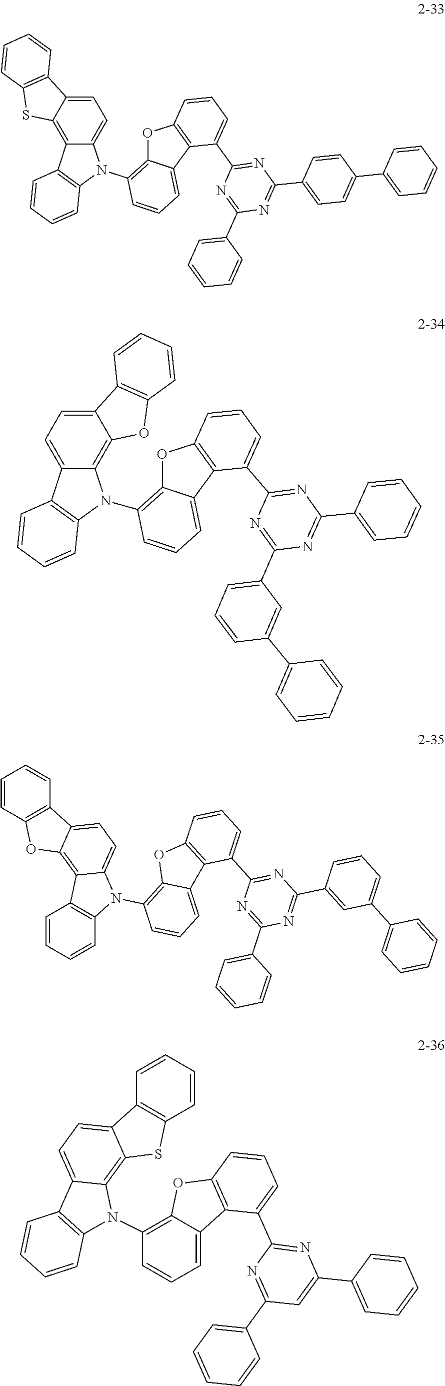 [SMILES: c1ccc(-c2ccc(-c3nc(-c4ccccc4)nc(-c4cccc5oc6c(-n7c8ccccc8c8c9sc%10ccccc%10c9ccc87)cccc6c45)n3)cc2)cc1 ; c1ccc(-c2cccc(-c3nc(-c4ccccc4)nc(-c4cccc5oc6c(-n7c8ccccc8c8ccc9c%10ccccc%10oc9c87)cccc6c45)n3)c2)cc1 ; c1ccc(-c2cccc(-c3nc(-c4ccccc4)nc(-c4cccc5oc6c(-n7c8ccccc8c8c9oc%10ccccc%10c9ccc87)cccc6c45)n3)c2)cc1 ; c1ccc(-c2cc(-c3ccccc3)nc(-c3cccc4oc5c(-n6c7ccccc7c7ccc8c9ccccc9sc8c76)cccc5c34)n2)cc1]

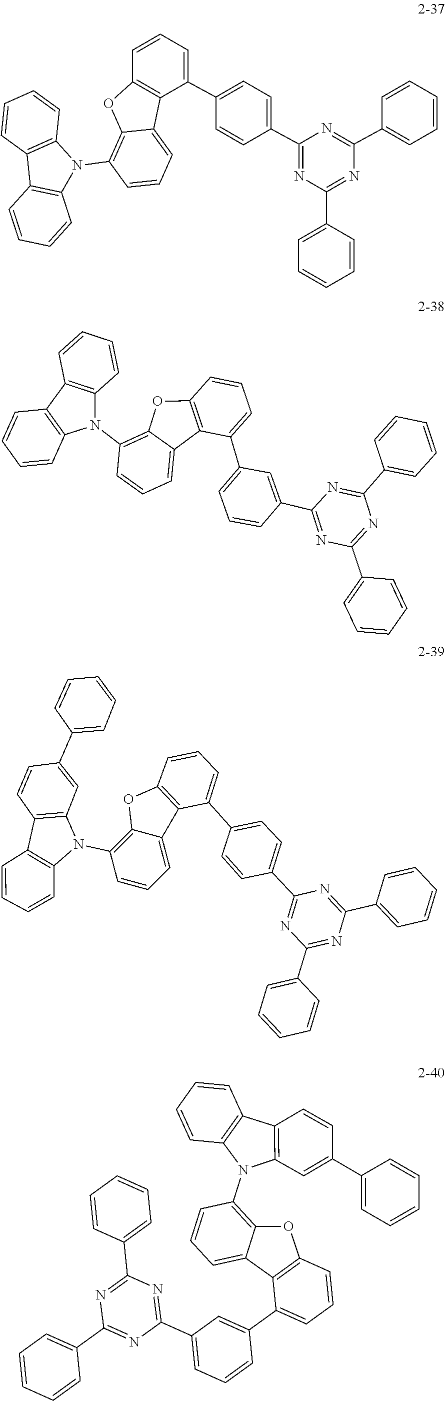 [SMILES: c1ccc(-c2nc(-c3ccccc3)nc(-c3ccc(-c4cccc5oc6c(-n7c8ccccc8c8ccccc87)cccc6c45)cc3)n2)cc1 ; c1ccc(-c2nc(-c3ccccc3)nc(-c3cccc(-c4cccc5oc6c(-n7c8ccccc8c8ccccc87)cccc6c45)c3)n2)cc1 ; c1ccc(-c2ccc3c4ccccc4n(-c4cccc5c4oc4cccc(-c6ccc(-c7nc(-c8ccccc8)nc(-c8ccccc8)n7)cc6)c45)c3c2)cc1 ; c1ccc(-c2ccc3c4ccccc4n(-c4cccc5c4oc4cccc(-c6cccc(-c7nc(-c8ccccc8)nc(-c8ccccc8)n7)c6)c45)c3c2)cc1]

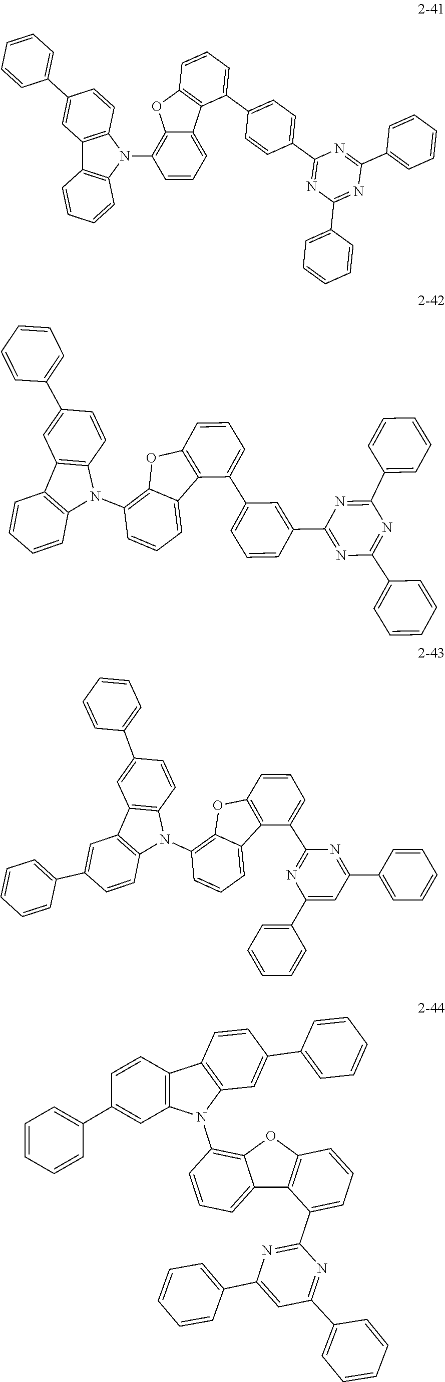 [SMILES: c1ccc(-c2ccc3c(c2)c2ccccc2n3-c2cccc3c2oc2cccc(-c4ccc(-c5nc(-c6ccccc6)nc(-c6ccccc6)n5)cc4)c23)cc1 ; c1ccc(-c2ccc3c(c2)c2ccccc2n3-c2cccc3c2oc2cccc(-c4cccc(-c5nc(-c6ccccc6)nc(-c6ccccc6)n5)c4)c23)cc1 ; c1ccc(-c2ccc3c(c2)c2cc(-c4ccccc4)ccc2n3-c2cccc3c2oc2cccc(-c4nc(-c5ccccc5)cc(-c5ccccc5)n4)c23)cc1 ; c1ccc(-c2ccc3c4ccc(-c5ccccc5)cc4n(-c4cccc5c4oc4cccc(-c6nc(-c7ccccc7)cc(-c7ccccc7)n6)c45)c3c2)cc1]

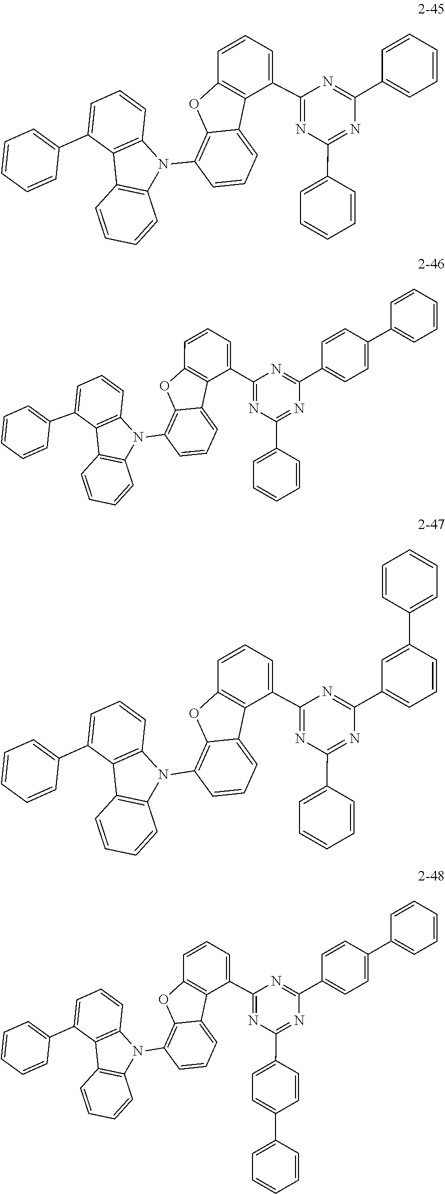 [SMILES: c1ccc(-c2nc(-c3ccccc3)nc(-c3cccc4oc5c(-n6c7ccccc7c7c(-c8ccccc8)cccc76)cccc5c34)n2)cc1 ; c1ccc(-c2ccc(-c3nc(-c4ccccc4)nc(-c4cccc5oc6c(-n7c8ccccc8c8c(-c9ccccc9)cccc87)cccc6c45)n3)cc2)cc1 ; c1ccc(-c2cccc(-c3nc(-c4ccccc4)nc(-c4cccc5oc6c(-n7c8ccccc8c8c(-c9ccccc9)cccc87)cccc6c45)n3)c2)cc1 ; c1ccc(-c2ccc(-c3nc(-c4ccc(-c5ccccc5)cc4)nc(-c4cccc5oc6c(-n7c8ccccc8c8c(-c9ccccc9)cccc87)cccc6c45)n3)cc2)cc1]

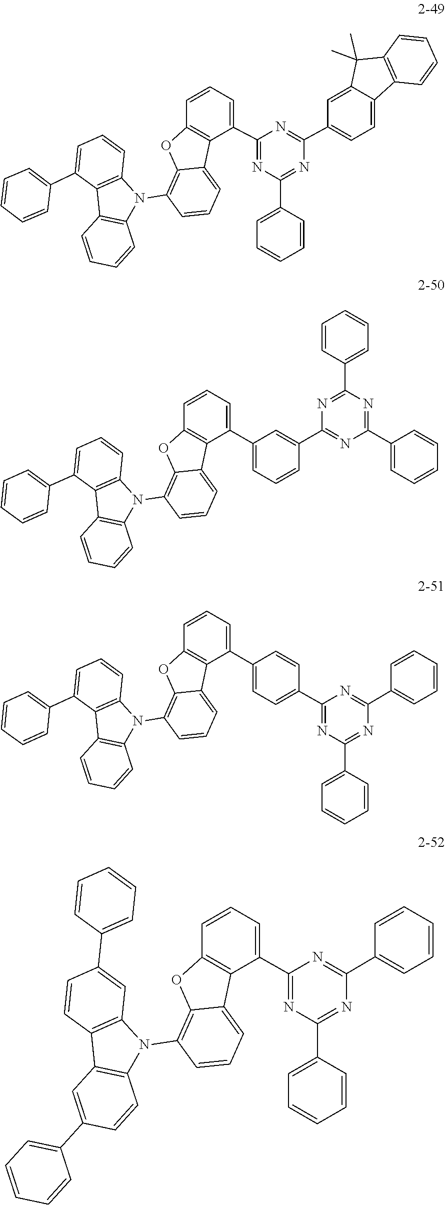 [SMILES: CC1(C)c2ccccc2-c2ccc(-c3nc(-c4ccccc4)nc(-c4cccc5oc6c(-n7c8ccccc8c8c(-c9ccccc9)cccc87)cccc6c45)n3)cc21 ; c1ccc(-c2nc(-c3ccccc3)nc(-c3cccc(-c4cccc5oc6c(-n7c8ccccc8c8c(-c9ccccc9)cccc87)cccc6c45)c3)n2)cc1 ; c1ccc(-c2nc(-c3ccccc3)nc(-c3ccc(-c4cccc5oc6c(-n7c8ccccc8c8c(-c9ccccc9)cccc87)cccc6c45)cc3)n2)cc1 ; c1ccc(-c2ccc3c(c2)c2ccc(-c4ccccc4)cc2n3-c2cccc3c2oc2cccc(-c4nc(-c5ccccc5)nc(-c5ccccc5)n4)c23)cc1]

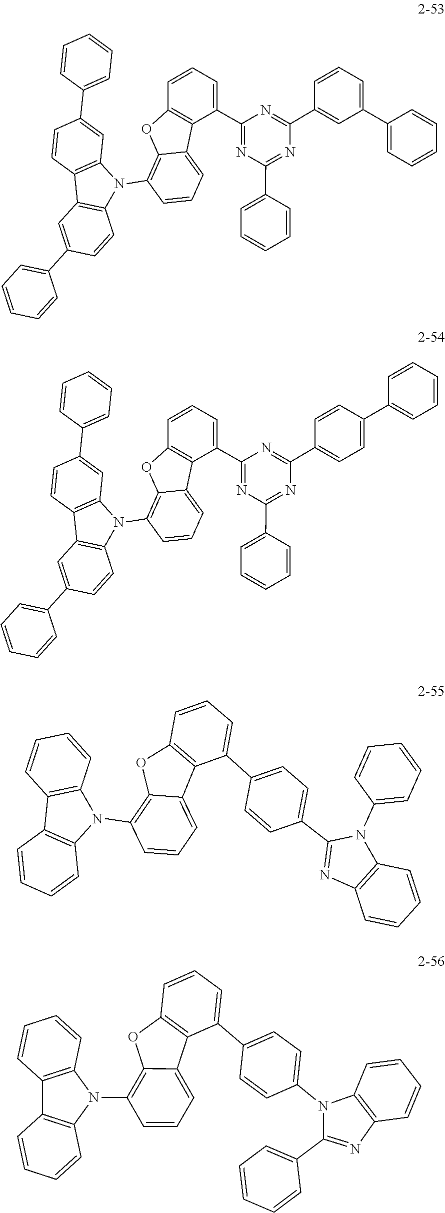 [SMILES: c1ccc(-c2cccc(-c3nc(-c4ccccc4)nc(-c4cccc5oc6c(-n7c8ccc(-c9ccccc9)cc8c8ccc(-c9ccccc9)cc87)cccc6c45)n3)c2)cc1 ; c1ccc(-c2ccc(-c3nc(-c4ccccc4)nc(-c4cccc5oc6c(-n7c8ccc(-c9ccccc9)cc8c8ccc(-c9ccccc9)cc87)cccc6c45)n3)cc2)cc1 ; c1ccc(-n2c(-c3ccc(-c4cccc5oc6c(-n7c8ccccc8c8ccccc87)cccc6c45)cc3)nc3ccccc32)cc1 ; c1ccc(-c2nc3ccccc3n2-c2ccc(-c3cccc4oc5c(-n6c7ccccc7c7ccccc76)cccc5c34)cc2)cc1]

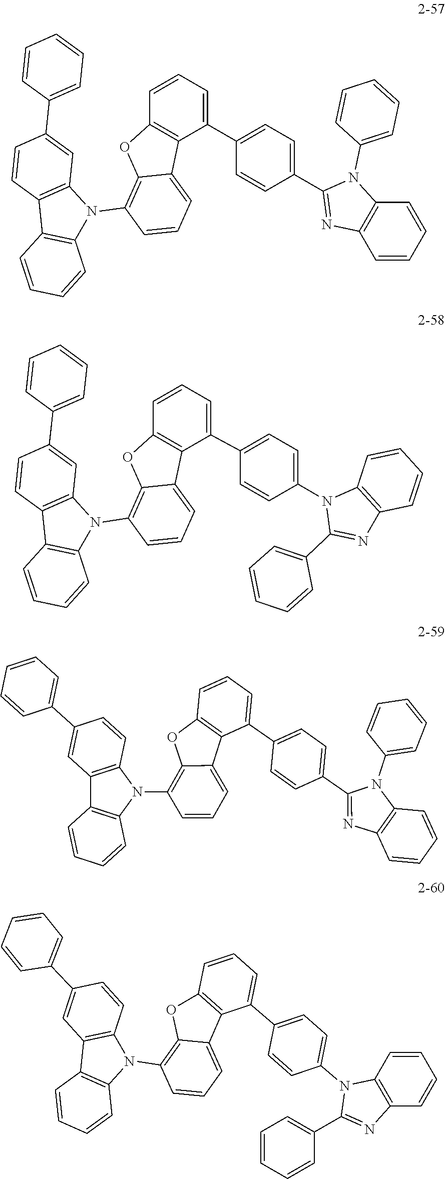 [SMILES: c1ccc(-c2ccc3c4ccccc4n(-c4cccc5c4oc4cccc(-c6ccc(-c7nc8ccccc8n7-c7ccccc7)cc6)c45)c3c2)cc1 ; c1ccc(-c2ccc3c4ccccc4n(-c4cccc5c4oc4cccc(-c6ccc(-n7c(-c8ccccc8)nc8ccccc87)cc6)c45)c3c2)cc1 ; c1ccc(-c2ccc3c(c2)c2ccccc2n3-c2cccc3c2oc2cccc(-c4ccc(-c5nc6ccccc6n5-c5ccccc5)cc4)c23)cc1 ; c1ccc(-c2ccc3c(c2)c2ccccc2n3-c2cccc3c2oc2cccc(-c4ccc(-n5c(-c6ccccc6)nc6ccccc65)cc4)c23)cc1]

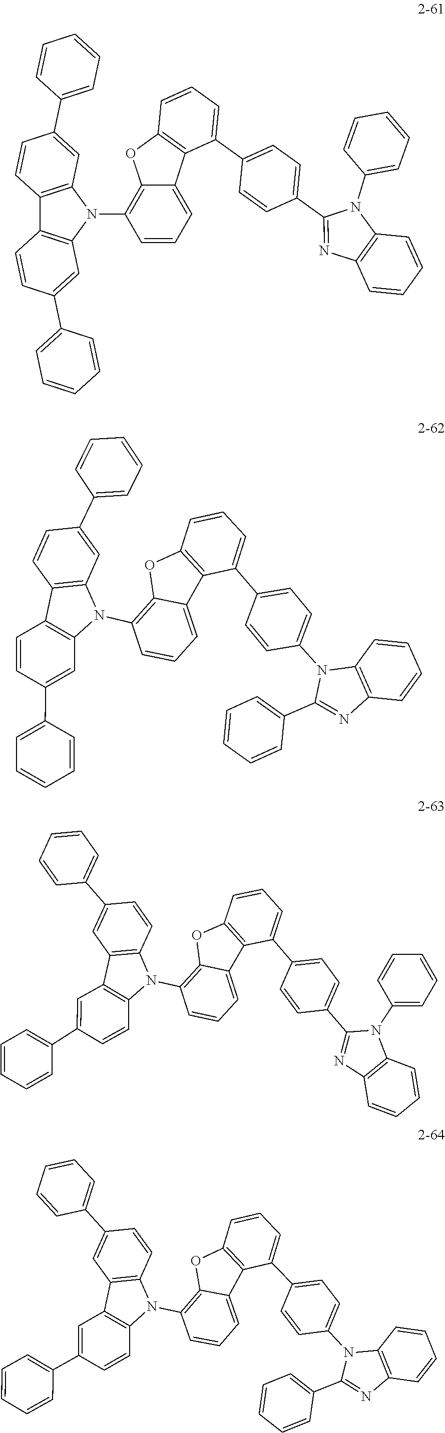 [SMILES: c1ccc(-c2ccc3c4ccc(-c5ccccc5)cc4n(-c4cccc5c4oc4cccc(-c6ccc(-c7nc8ccccc8n7-c7ccccc7)cc6)c45)c3c2)cc1 ; c1ccc(-c2ccc3c4ccc(-c5ccccc5)cc4n(-c4cccc5c4oc4cccc(-c6ccc(-n7c(-c8ccccc8)nc8ccccc87)cc6)c45)c3c2)cc1 ; c1ccc(-c2ccc3c(c2)c2cc(-c4ccccc4)ccc2n3-c2cccc3c2oc2cccc(-c4ccc(-c5nc6ccccc6n5-c5ccccc5)cc4)c23)cc1 ; c1ccc(-c2ccc3c(c2)c2cc(-c4ccccc4)ccc2n3-c2cccc3c2oc2cccc(-c4ccc(-n5c(-c6ccccc6)nc6ccccc65)cc4)c23)cc1]

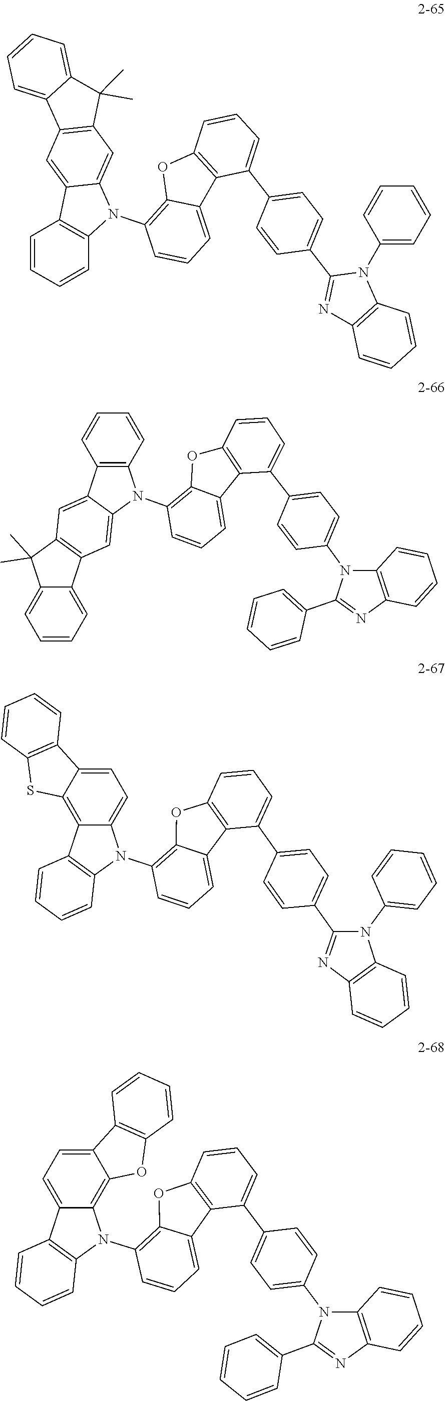 [SMILES: CC1(C)c2ccccc2-c2cc3c4ccccc4n(-c4cccc5c4oc4cccc(-c6ccc(-c7nc8ccccc8n7-c7ccccc7)cc6)c45)c3cc21 ; CC1(C)c2ccccc2-c2cc3c(cc21)c1ccccc1n3-c1cccc2c1oc1cccc(-c3ccc(-n4c(-c5ccccc5)nc5ccccc54)cc3)c12 ; c1ccc(-n2c(-c3ccc(-c4cccc5oc6c(-n7c8ccccc8c8c9sc%10ccccc%10c9ccc87)cccc6c45)cc3)nc3ccccc32)cc1 ; c1ccc(-c2nc3ccccc3n2-c2ccc(-c3cccc4oc5c(-n6c7ccccc7c7ccc8c9ccccc9oc8c76)cccc5c34)cc2)cc1]

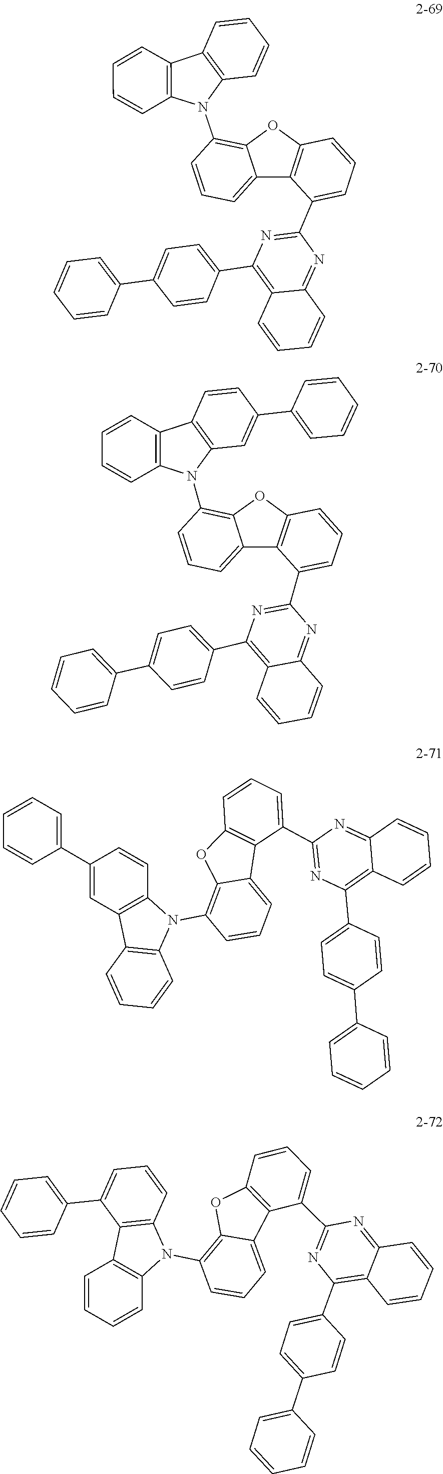

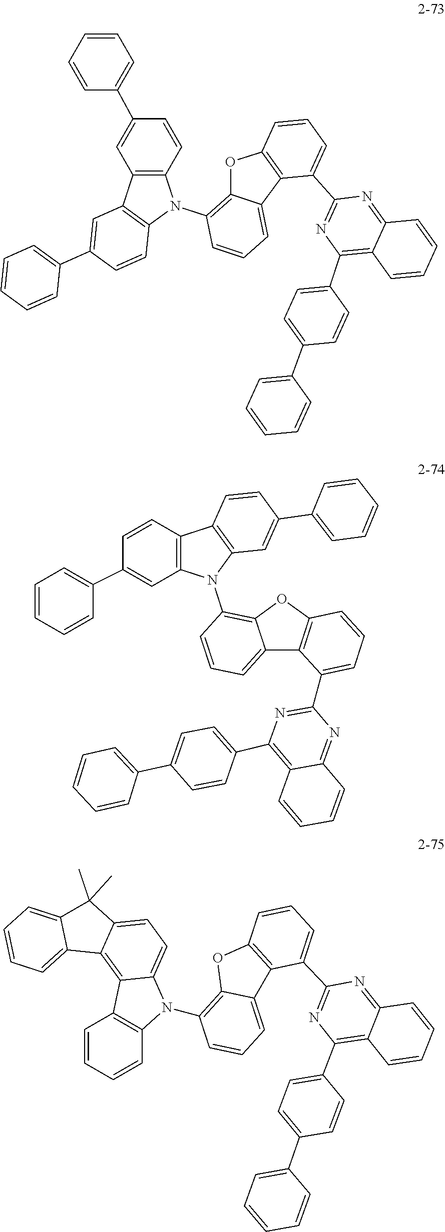 [SMILES: c1ccc(-c2ccc(-c3nc(-c4cccc5oc6c(-n7c8ccc(-c9ccccc9)cc8c8cc(-c9ccccc9)ccc87)cccc6c45)nc4ccccc34)cc2)cc1 ; c1ccc(-c2ccc(-c3nc(-c4cccc5oc6c(-n7c8cc(-c9ccccc9)ccc8c8ccc(-c9ccccc9)cc87)cccc6c45)nc4ccccc34)cc2)cc1 ; CC1(C)c2ccccc2-c2c1ccc1c2c2ccccc2n1-c1cccc2c1oc1cccc(-c3nc(-c4ccc(-c5ccccc5)cc4)c4ccccc4n3)c12]

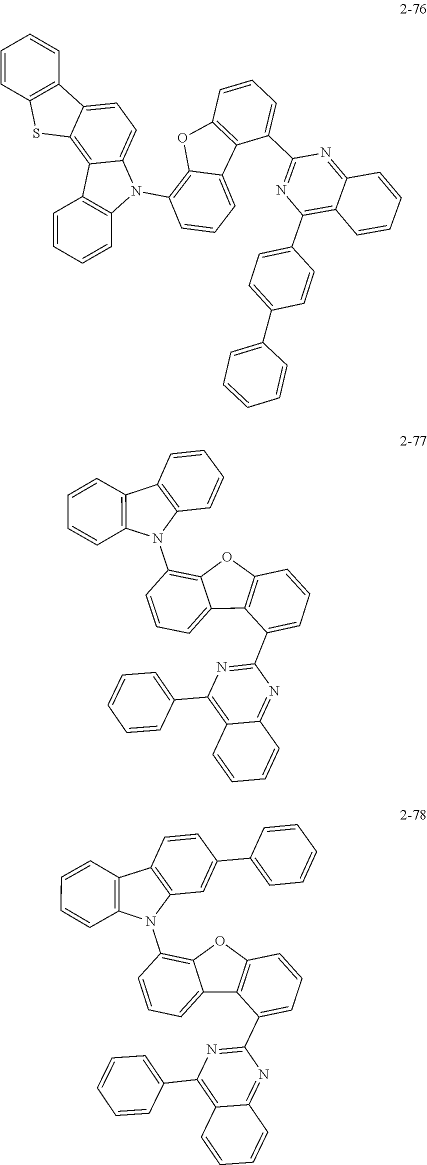 [SMILES: c1ccc(-c2ccc(-c3nc(-c4cccc5oc6c(-n7c8ccccc8c8c9sc%10ccccc%10c9ccc87)cccc6c45)nc4ccccc34)cc2)cc1 ; c1ccc(-c2nc(-c3cccc4oc5c(-n6c7ccccc7c7ccccc76)cccc5c34)nc3ccccc23)cc1 ; c1ccc(-c2ccc3c4ccccc4n(-c4cccc5c4oc4cccc(-c6nc(-c7ccccc7)c7ccccc7n6)c45)c3c2)cc1]

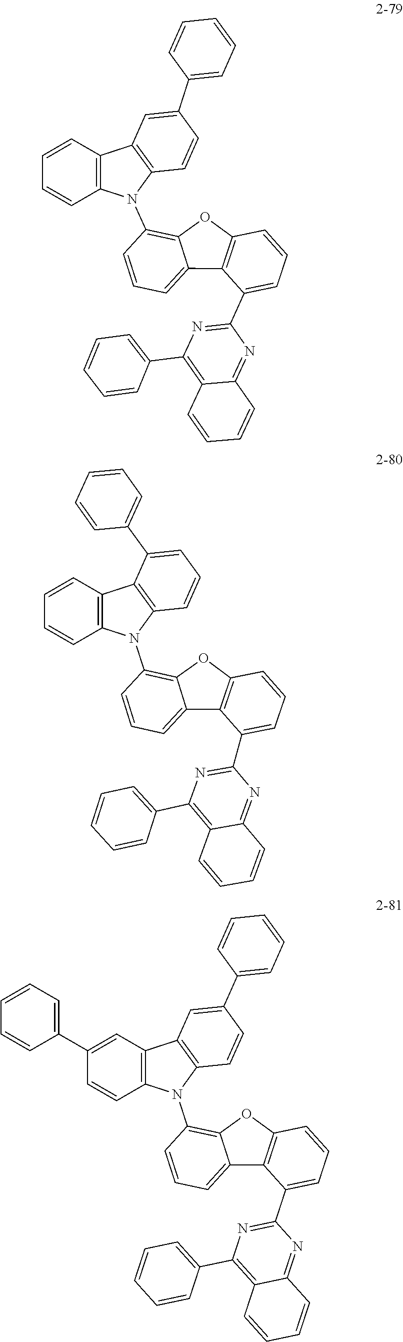

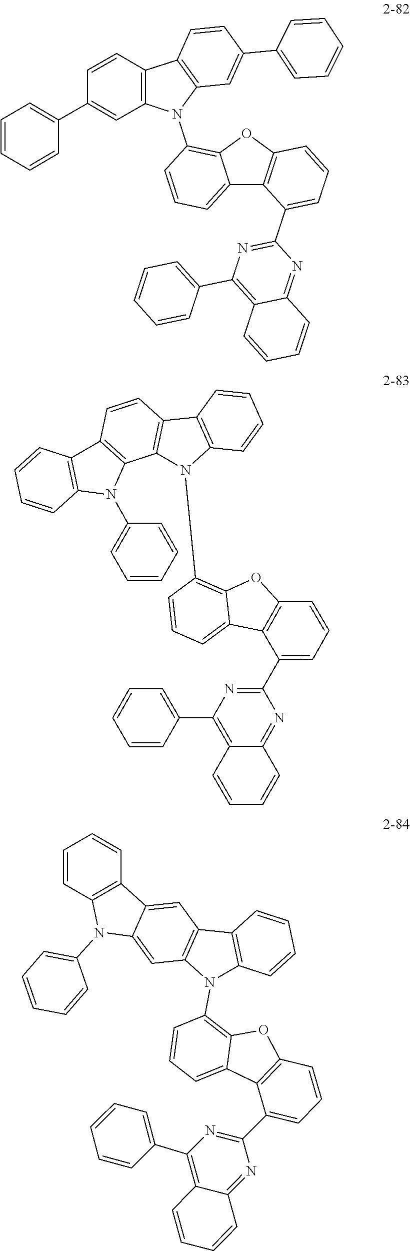

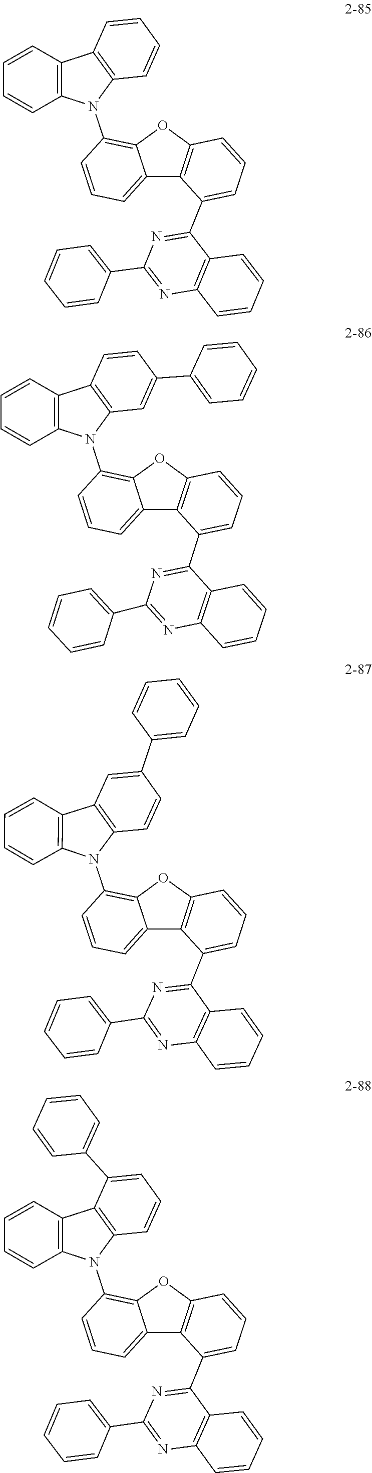

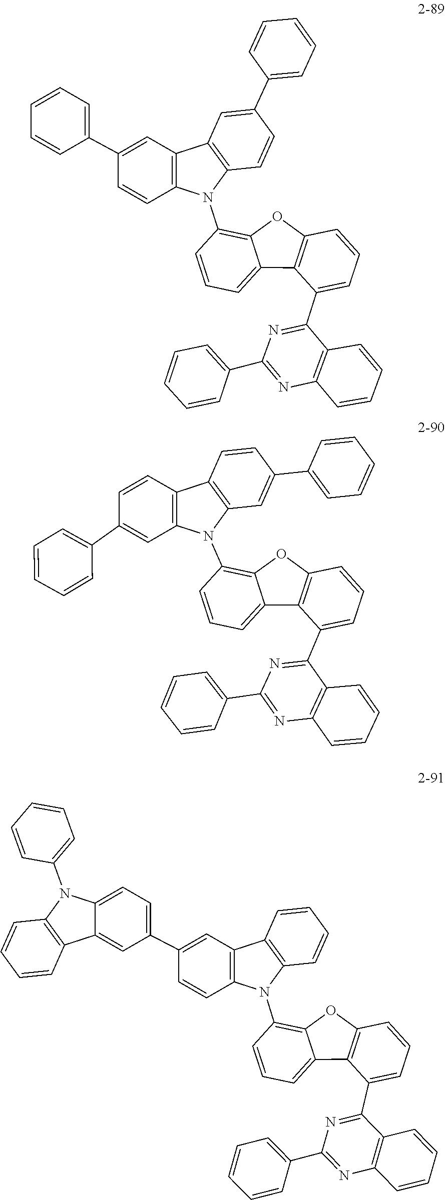

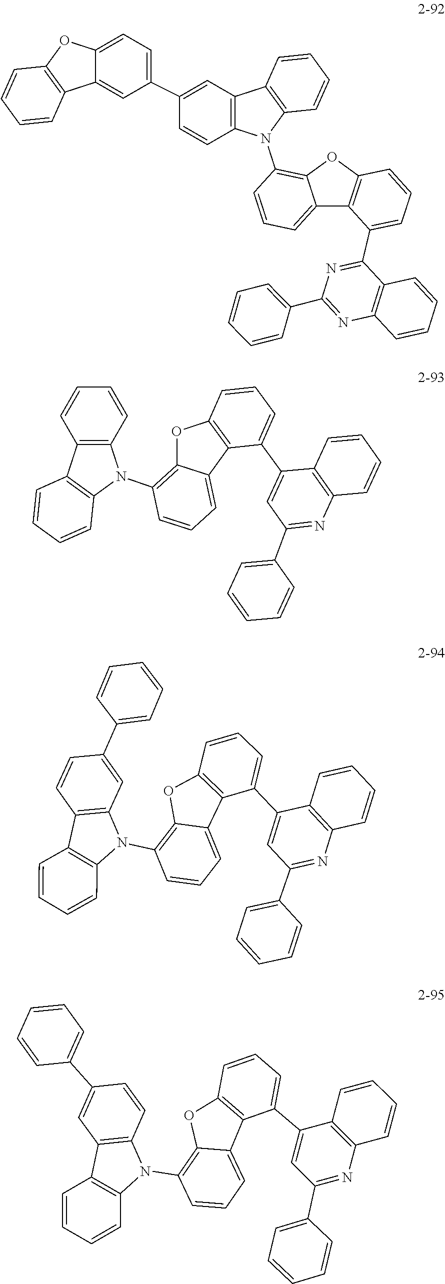

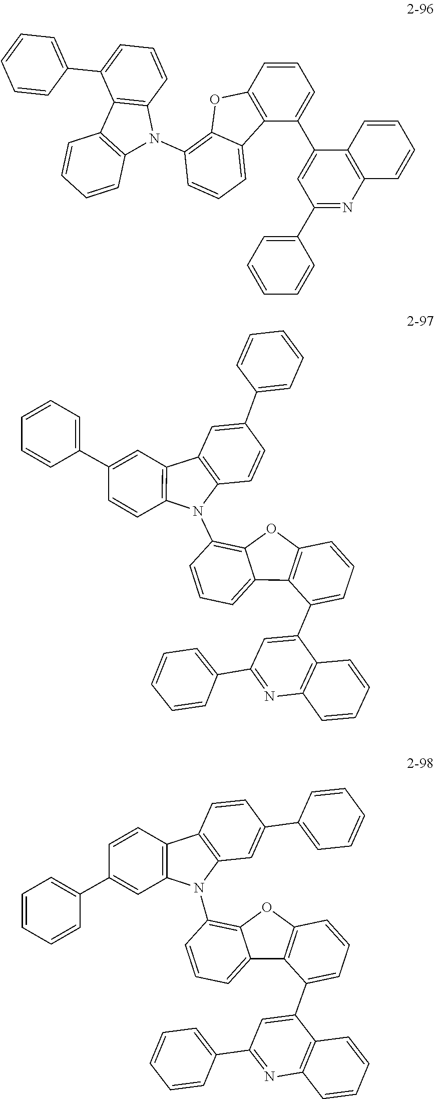

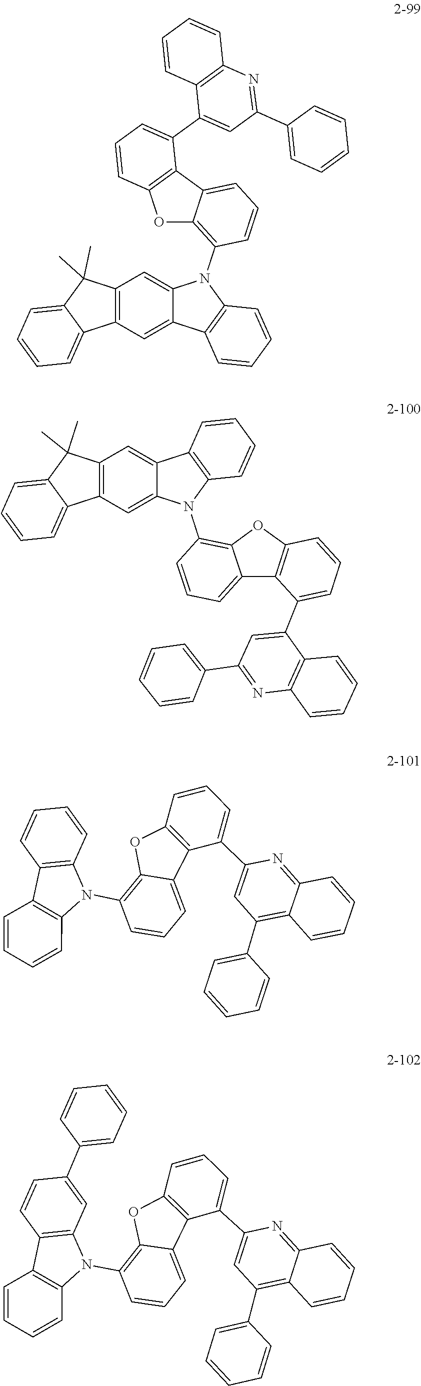 [SMILES: CC1(C)c2ccccc2-c2cc3c4ccccc4n(-c4cccc5c4oc4cccc(-c6cc(-c7ccccc7)nc7ccccc67)c45)c3cc21 ; CC1(C)c2ccccc2-c2cc3c(cc21)c1ccccc1n3-c1cccc2c1oc1cccc(-c3cc(-c4ccccc4)nc4ccccc34)c12 ; c1ccc(-c2cc(-c3cccc4oc5c(-n6c7ccccc7c7ccccc76)cccc5c34)nc3ccccc23)cc1 ; c1ccc(-c2ccc3c4ccccc4n(-c4cccc5c4oc4cccc(-c6cc(-c7ccccc7)c7ccccc7n6)c45)c3c2)cc1]

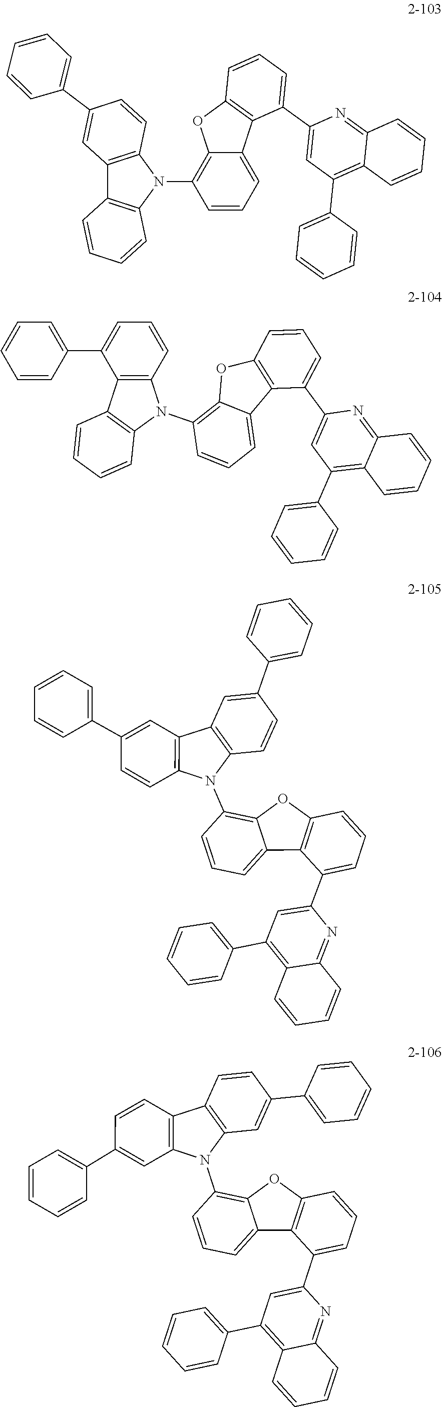

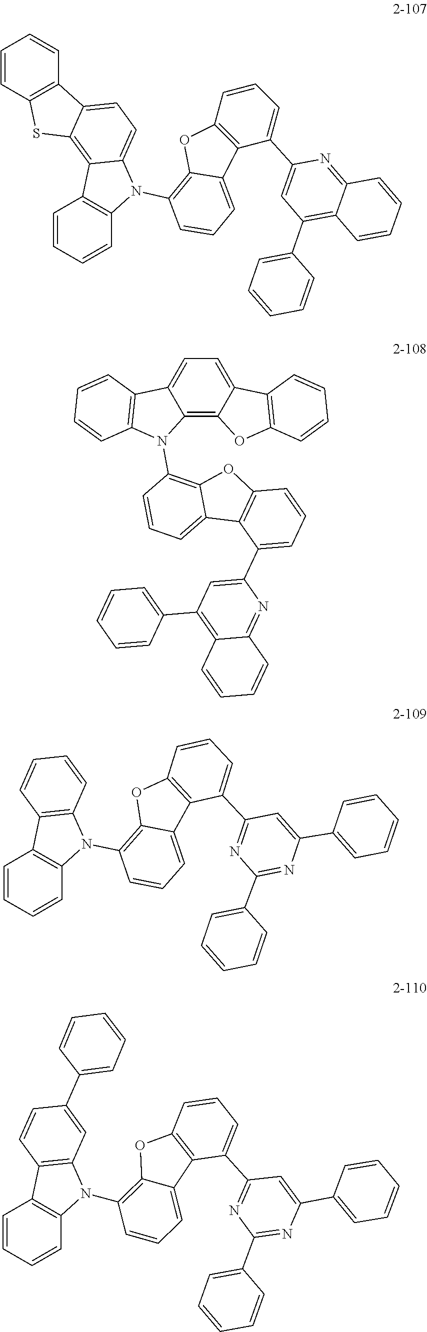

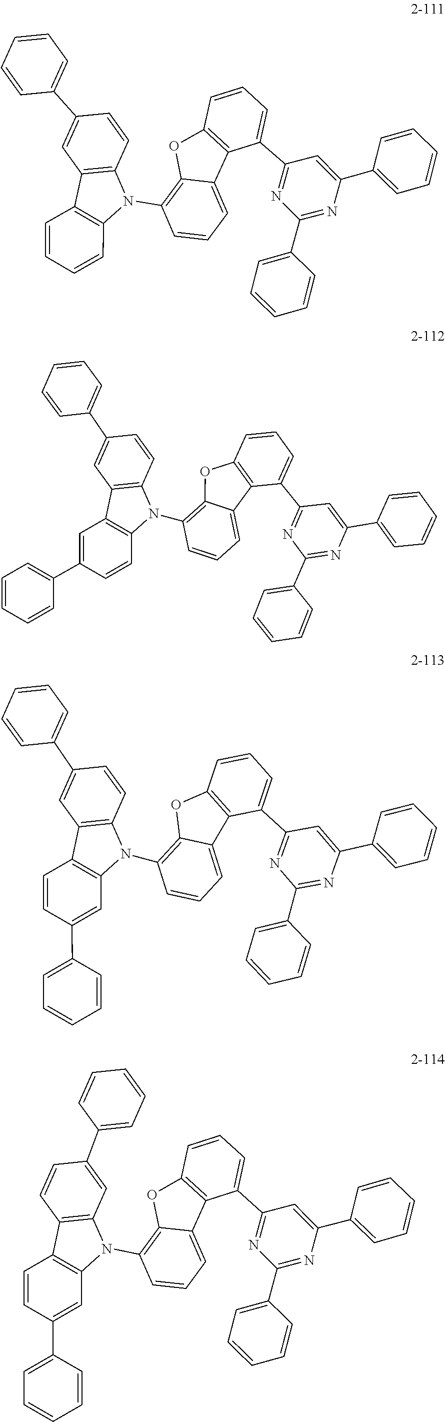 [SMILES: c1ccc(-c2ccc3c(c2)c2ccccc2n3-c2cccc3c2oc2cccc(-c4cc(-c5ccccc5)nc(-c5ccccc5)n4)c23)cc1 ; c1ccc(-c2ccc3c(c2)c2cc(-c4ccccc4)ccc2n3-c2cccc3c2oc2cccc(-c4cc(-c5ccccc5)nc(-c5ccccc5)n4)c23)cc1 ; c1ccc(-c2ccc3c(c2)c2ccc(-c4ccccc4)cc2n3-c2cccc3c2oc2cccc(-c4cc(-c5ccccc5)nc(-c5ccccc5)n4)c23)cc1 ; c1ccc(-c2ccc3c4ccc(-c5ccccc5)cc4n(-c4cccc5c4oc4cccc(-c6cc(-c7ccccc7)nc(-c7ccccc7)n6)c45)c3c2)cc1]

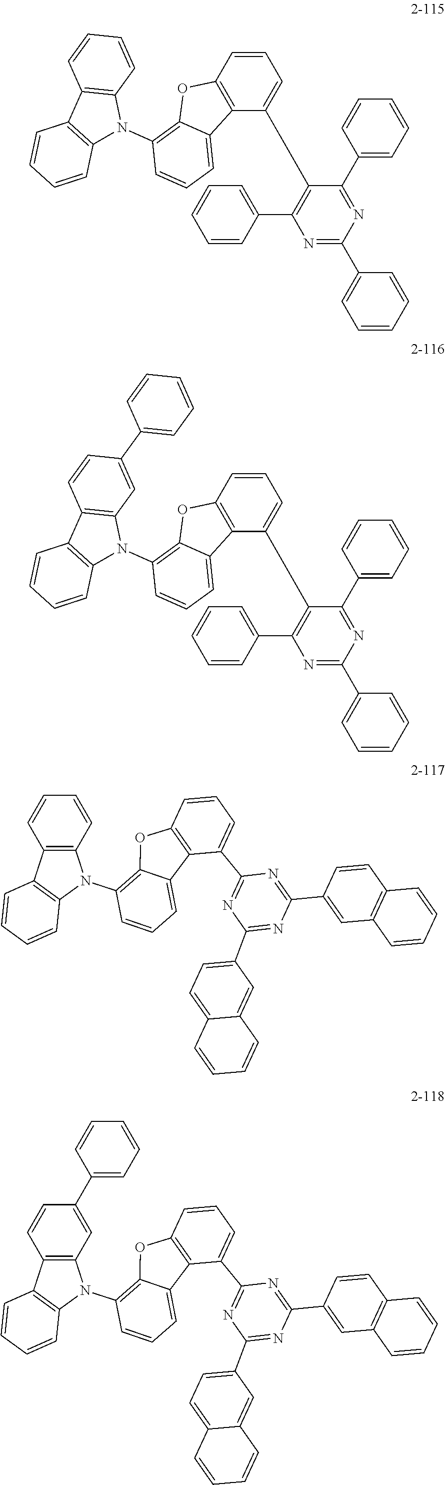 [SMILES: c1ccc(-c2nc(-c3ccccc3)c(-c3cccc4oc5c(-n6c7ccccc7c7ccccc76)cccc5c34)c(-c3ccccc3)n2)cc1 ; c1ccc(-c2ccc3c4ccccc4n(-c4cccc5c4oc4cccc(-c6c(-c7ccccc7)nc(-c7ccccc7)nc6-c6ccccc6)c45)c3c2)cc1 ; c1ccc2cc(-c3nc(-c4ccc5ccccc5c4)nc(-c4cccc5oc6c(-n7c8ccccc8c8ccccc87)cccc6c45)n3)ccc2c1 ; c1ccc(-c2ccc3c4ccccc4n(-c4cccc5c4oc4cccc(-c6nc(-c7ccc8ccccc8c7)nc(-c7ccc8ccccc8c7)n6)c45)c3c2)cc1]

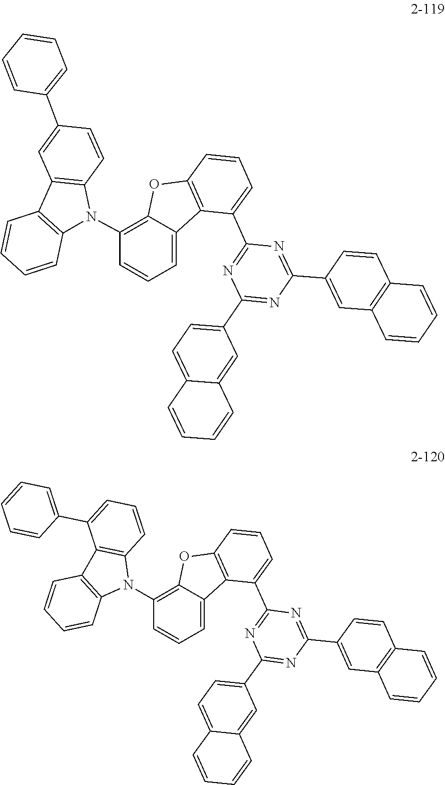 [SMILES: c1ccc(-c2ccc3c(c2)c2ccccc2n3-c2cccc3c2oc2cccc(-c4nc(-c5ccc6ccccc6c5)nc(-c5ccc6ccccc6c5)n4)c23)cc1 ; c1ccc(-c2cccc3c2c2ccccc2n3-c2cccc3c2oc2cccc(-c4nc(-c5ccc6ccccc6c5)nc(-c5ccc6ccccc6c5)n4)c23)cc1]

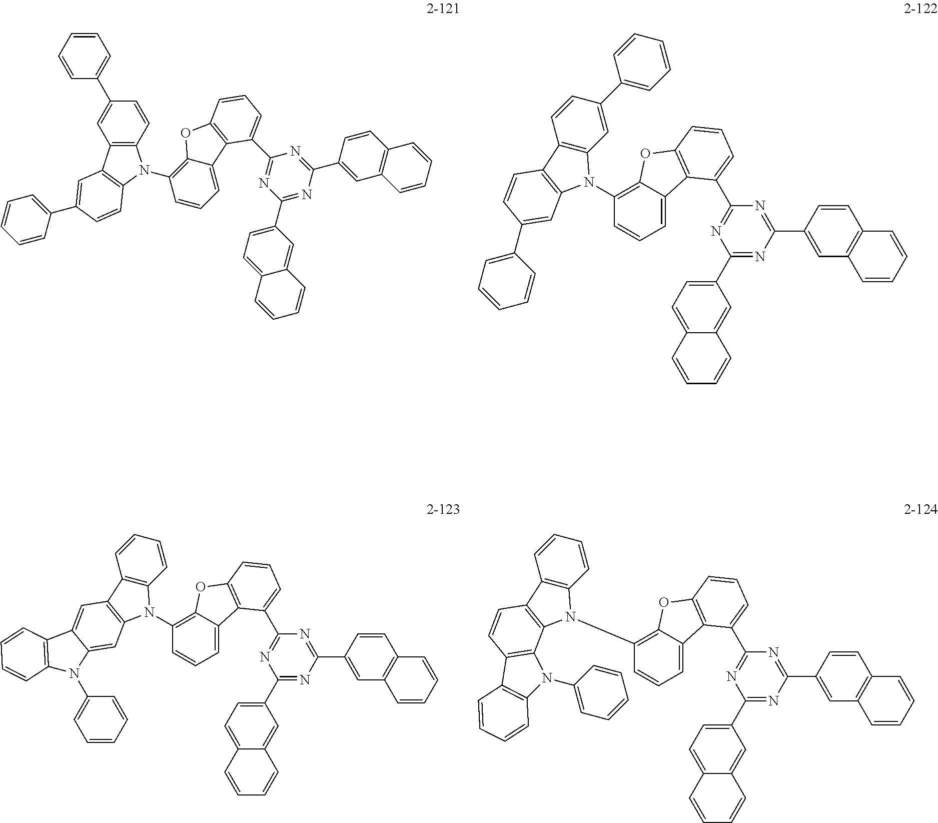 [SMILES: c1ccc(-c2ccc3c(c2)c2cc(-c4ccccc4)ccc2n3-c2cccc3c2oc2cccc(-c4nc(-c5ccc6ccccc6c5)nc(-c5ccc6ccccc6c5)n4)c23)cc1 ; c1ccc(-c2ccc3c4ccc(-c5ccccc5)cc4n(-c4cccc5c4oc4cccc(-c6nc(-c7ccc8ccccc8c7)nc(-c7ccc8ccccc8c7)n6)c45)c3c2)cc1 ; c1ccc(-n2c3ccccc3c3cc4c5ccccc5n(-c5cccc6c5oc5cccc(-c7nc(-c8ccc9ccccc9c8)nc(-c8ccc9ccccc9c8)n7)c56)c4cc32)cc1 ; c1ccc(-n2c3ccccc3c3ccc4c5ccccc5n(-c5cccc6c5oc5cccc(-c7nc(-c8ccc9ccccc9c8)nc(-c8ccc9ccccc9c8)n7)c56)c4c32)cc1]

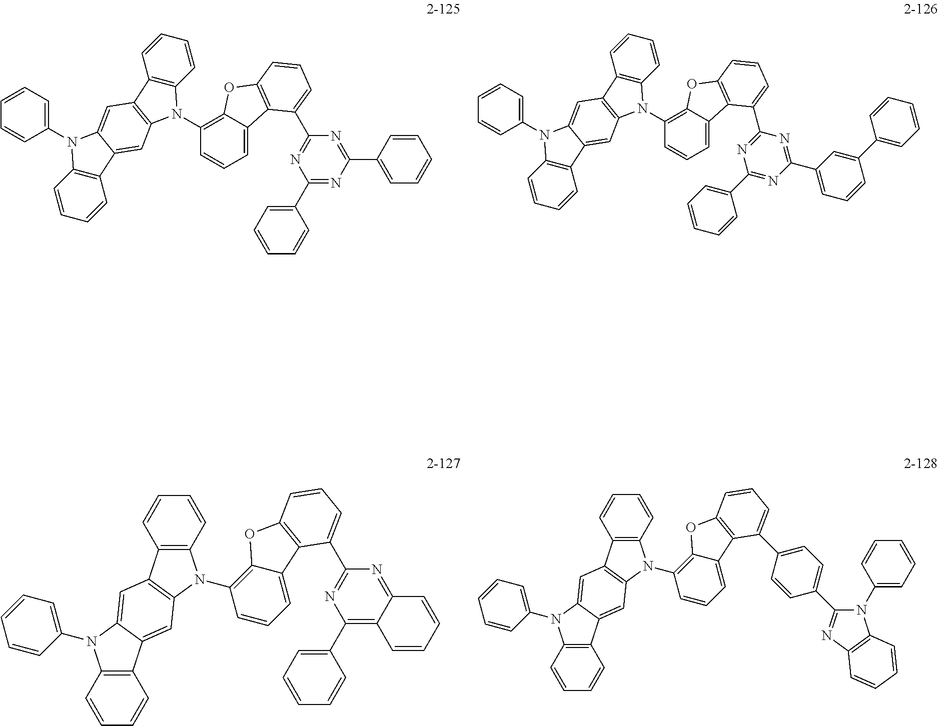 [SMILES: c1ccc(-c2nc(-c3ccccc3)nc(-c3cccc4oc5c(-n6c7ccccc7c7cc8c(cc76)c6ccccc6n8-c6ccccc6)cccc5c34)n2)cc1 ; c1ccc(-c2cccc(-c3nc(-c4ccccc4)nc(-c4cccc5oc6c(-n7c8ccccc8c8cc9c(cc87)c7ccccc7n9-c7ccccc7)cccc6c45)n3)c2)cc1 ; c1ccc(-c2nc(-c3cccc4oc5c(-n6c7ccccc7c7cc8c(cc76)c6ccccc6n8-c6ccccc6)cccc5c34)nc3ccccc23)cc1 ; c1ccc(-n2c(-c3ccc(-c4cccc5oc6c(-n7c8ccccc8c8cc9c(cc87)c7ccccc7n9-c7ccccc7)cccc6c45)cc3)nc3ccccc32)cc1]

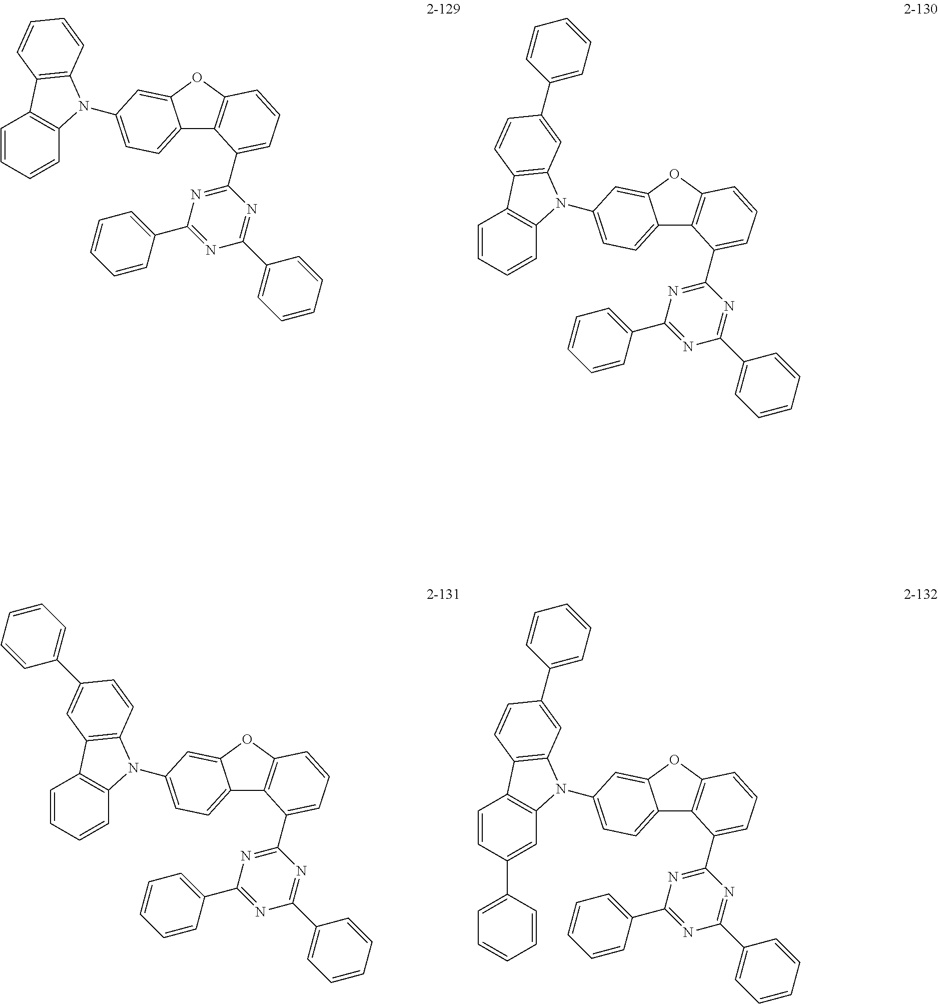 [SMILES: c1ccc(-c2nc(-c3ccccc3)nc(-c3cccc4oc5cc(-n6c7ccccc7c7ccccc76)ccc5c34)n2)cc1 ; c1ccc(-c2ccc3c4ccccc4n(-c4ccc5c(c4)oc4cccc(-c6nc(-c7ccccc7)nc(-c7ccccc7)n6)c45)c3c2)cc1 ; c1ccc(-c2ccc3c(c2)c2ccccc2n3-c2ccc3c(c2)oc2cccc(-c4nc(-c5ccccc5)nc(-c5ccccc5)n4)c23)cc1 ; c1ccc(-c2ccc3c4ccc(-c5ccccc5)cc4n(-c4ccc5c(c4)oc4cccc(-c6nc(-c7ccccc7)nc(-c7ccccc7)n6)c45)c3c2)cc1]

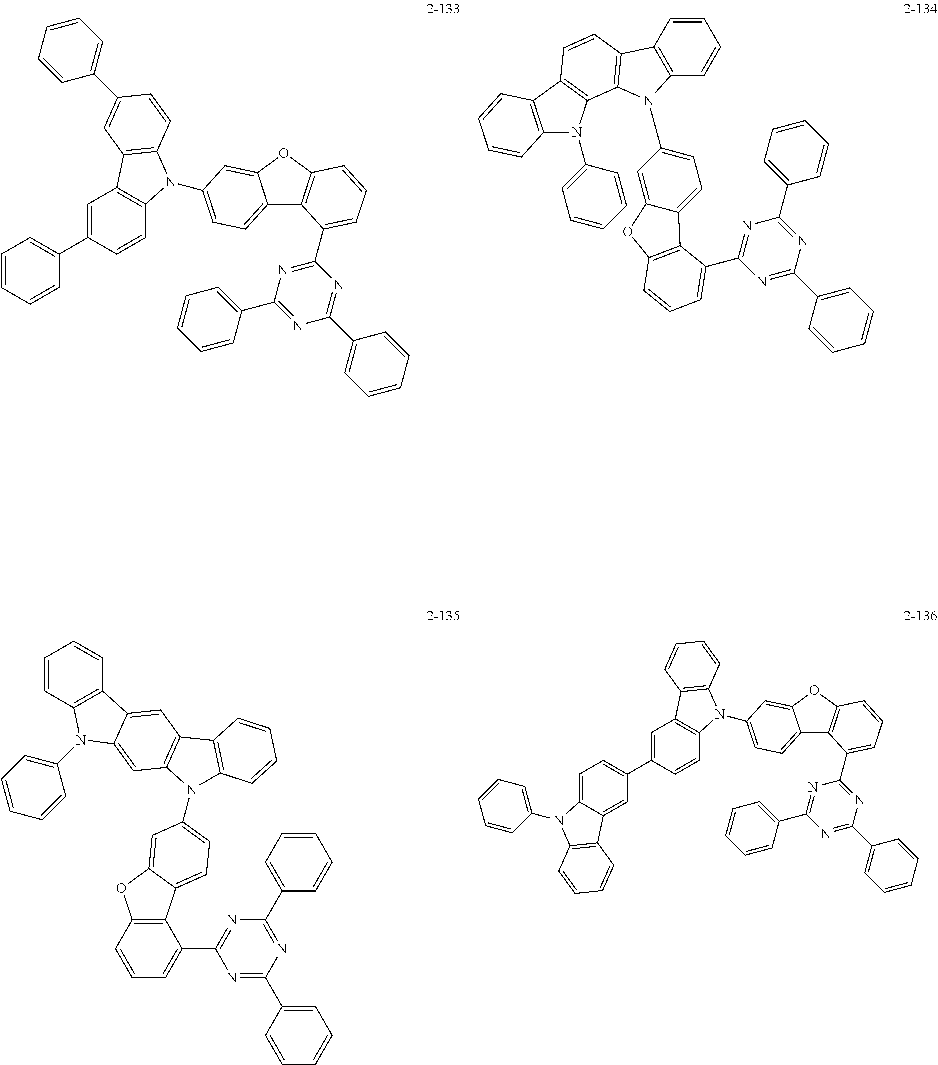 [SMILES: c1ccc(-c2ccc3c(c2)c2cc(-c4ccccc4)ccc2n3-c2ccc3c(c2)oc2cccc(-c4nc(-c5ccccc5)nc(-c5ccccc5)n4)c23)cc1 ; c1ccc(-c2nc(-c3ccccc3)nc(-c3cccc4oc5cc(-n6c7ccccc7c7ccc8c9ccccc9n(-c9ccccc9)c8c76)ccc5c34)n2)cc1 ; c1ccc(-c2nc(-c3ccccc3)nc(-c3cccc4oc5cc(-n6c7ccccc7c7cc8c9ccccc9n(-c9ccccc9)c8cc76)ccc5c34)n2)cc1 ; c1ccc(-c2nc(-c3ccccc3)nc(-c3cccc4oc5cc(-n6c7ccccc7c7cc(-c8ccc9c(c8)c8ccccc8n9-c8ccccc8)ccc76)ccc5c34)n2)cc1]

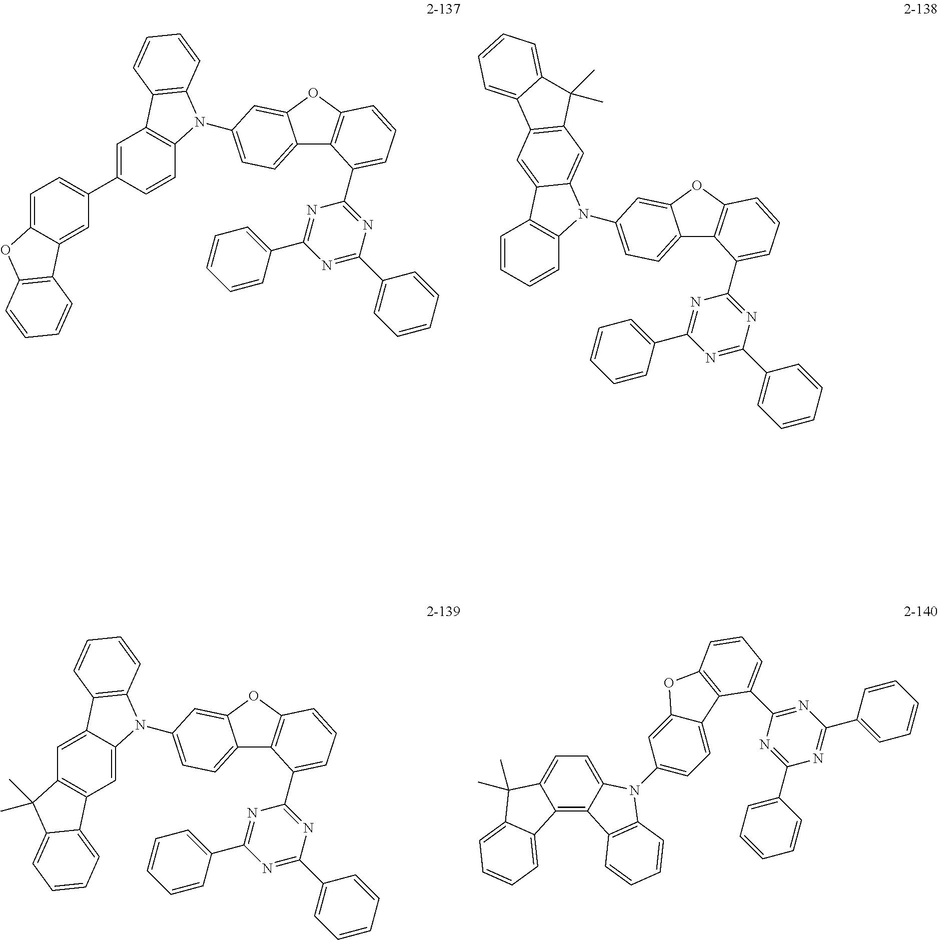 [SMILES: c1ccc(-c2nc(-c3ccccc3)nc(-c3cccc4oc5cc(-n6c7ccccc7c7cc(-c8ccc9oc%10ccccc%10c9c8)ccc76)ccc5c34)n2)cc1 ; CC1(C)c2ccccc2-c2cc3c4ccccc4n(-c4ccc5c(c4)oc4cccc(-c6nc(-c7ccccc7)nc(-c7ccccc7)n6)c45)c3cc21 ; CC1(C)c2ccccc2-c2cc3c(cc21)c1ccccc1n3-c1ccc2c(c1)oc1cccc(-c3nc(-c4ccccc4)nc(-c4ccccc4)n3)c12 ; CC1(C)c2ccccc2-c2c1ccc1c2c2ccccc2n1-c1ccc2c(c1)oc1cccc(-c3nc(-c4ccccc4)nc(-c4ccccc4)n3)c12]

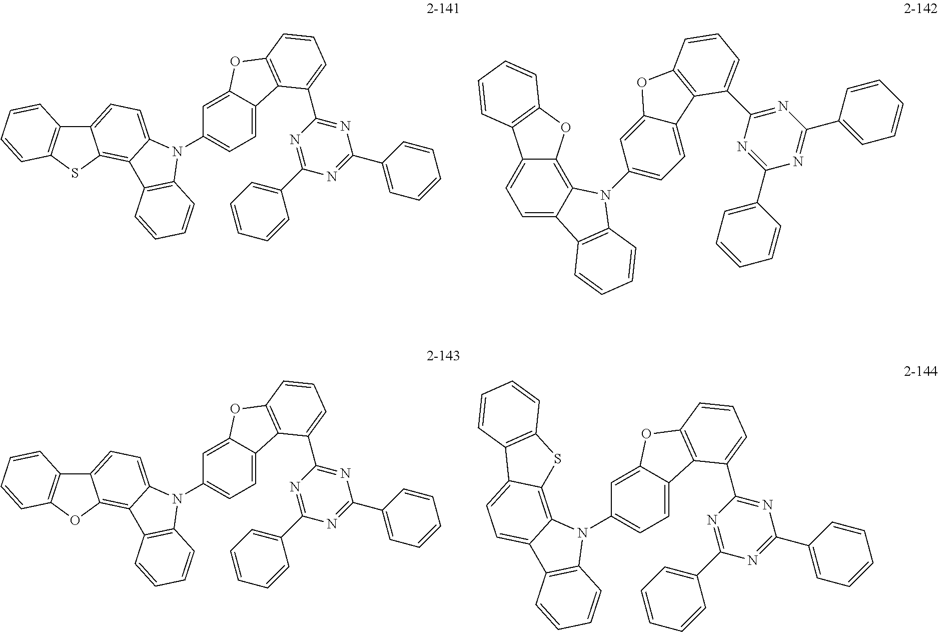 [SMILES: c1ccc(-c2nc(-c3ccccc3)nc(-c3cccc4oc5cc(-n6c7ccccc7c7c8sc9ccccc9c8ccc76)ccc5c34)n2)cc1 ; c1ccc(-c2nc(-c3ccccc3)nc(-c3cccc4oc5cc(-n6c7ccccc7c7ccc8c9ccccc9oc8c76)ccc5c34)n2)cc1 ; c1ccc(-c2nc(-c3ccccc3)nc(-c3cccc4oc5cc(-n6c7ccccc7c7c8oc9ccccc9c8ccc76)ccc5c34)n2)cc1 ; c1ccc(-c2nc(-c3ccccc3)nc(-c3cccc4oc5cc(-n6c7ccccc7c7ccc8c9ccccc9sc8c76)ccc5c34)n2)cc1]

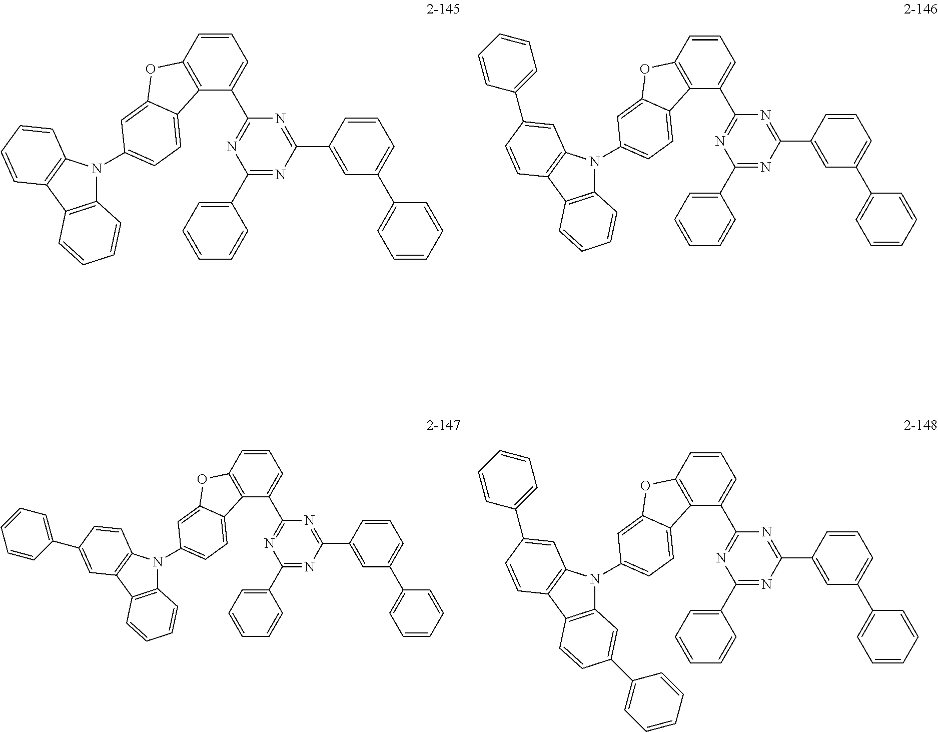 [SMILES: c1ccc(-c2cccc(-c3nc(-c4ccccc4)nc(-c4cccc5oc6cc(-n7c8ccccc8c8ccccc87)ccc6c45)n3)c2)cc1 ; c1ccc(-c2cccc(-c3nc(-c4ccccc4)nc(-c4cccc5oc6cc(-n7c8ccccc8c8ccc(-c9ccccc9)cc87)ccc6c45)n3)c2)cc1 ; c1ccc(-c2cccc(-c3nc(-c4ccccc4)nc(-c4cccc5oc6cc(-n7c8ccccc8c8cc(-c9ccccc9)ccc87)ccc6c45)n3)c2)cc1 ; c1ccc(-c2cccc(-c3nc(-c4ccccc4)nc(-c4cccc5oc6cc(-n7c8cc(-c9ccccc9)ccc8c8ccc(-c9ccccc9)cc87)ccc6c45)n3)c2)cc1]

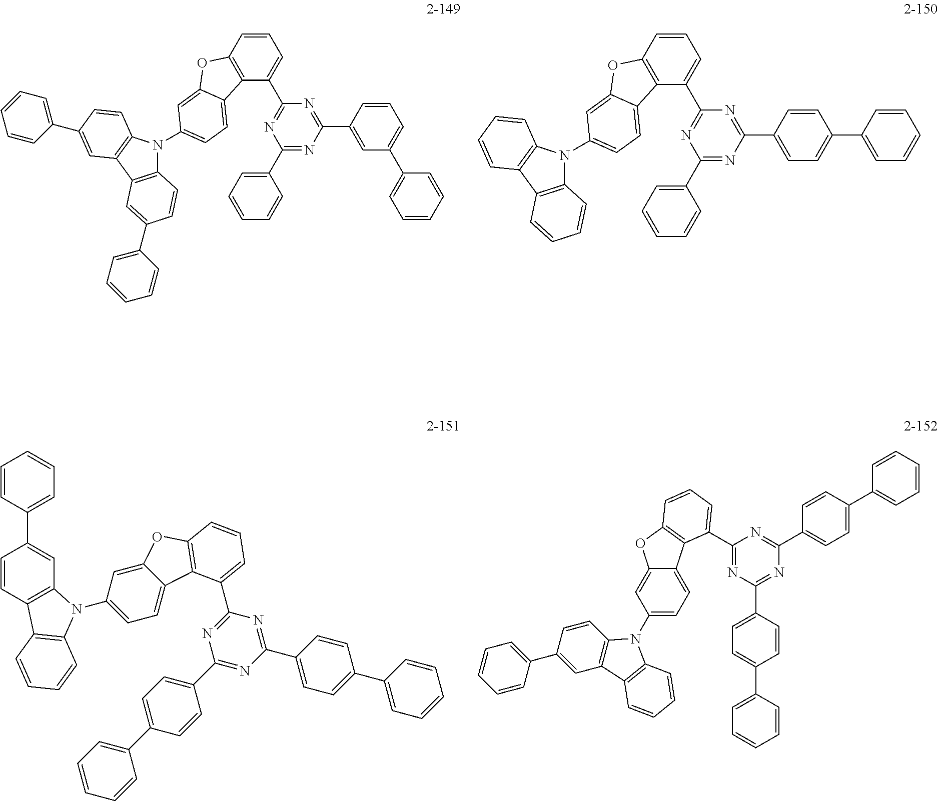 [SMILES: c1ccc(-c2cccc(-c3nc(-c4ccccc4)nc(-c4cccc5oc6cc(-n7c8ccc(-c9ccccc9)cc8c8cc(-c9ccccc9)ccc87)ccc6c45)n3)c2)cc1 ; c1ccc(-c2ccc(-c3nc(-c4ccccc4)nc(-c4cccc5oc6cc(-n7c8ccccc8c8ccccc87)ccc6c45)n3)cc2)cc1 ; c1ccc(-c2ccc(-c3nc(-c4ccc(-c5ccccc5)cc4)nc(-c4cccc5oc6cc(-n7c8ccccc8c8ccc(-c9ccccc9)cc87)ccc6c45)n3)cc2)cc1 ; c1ccc(-c2ccc(-c3nc(-c4ccc(-c5ccccc5)cc4)nc(-c4cccc5oc6cc(-n7c8ccccc8c8cc(-c9ccccc9)ccc87)ccc6c45)n3)cc2)cc1]

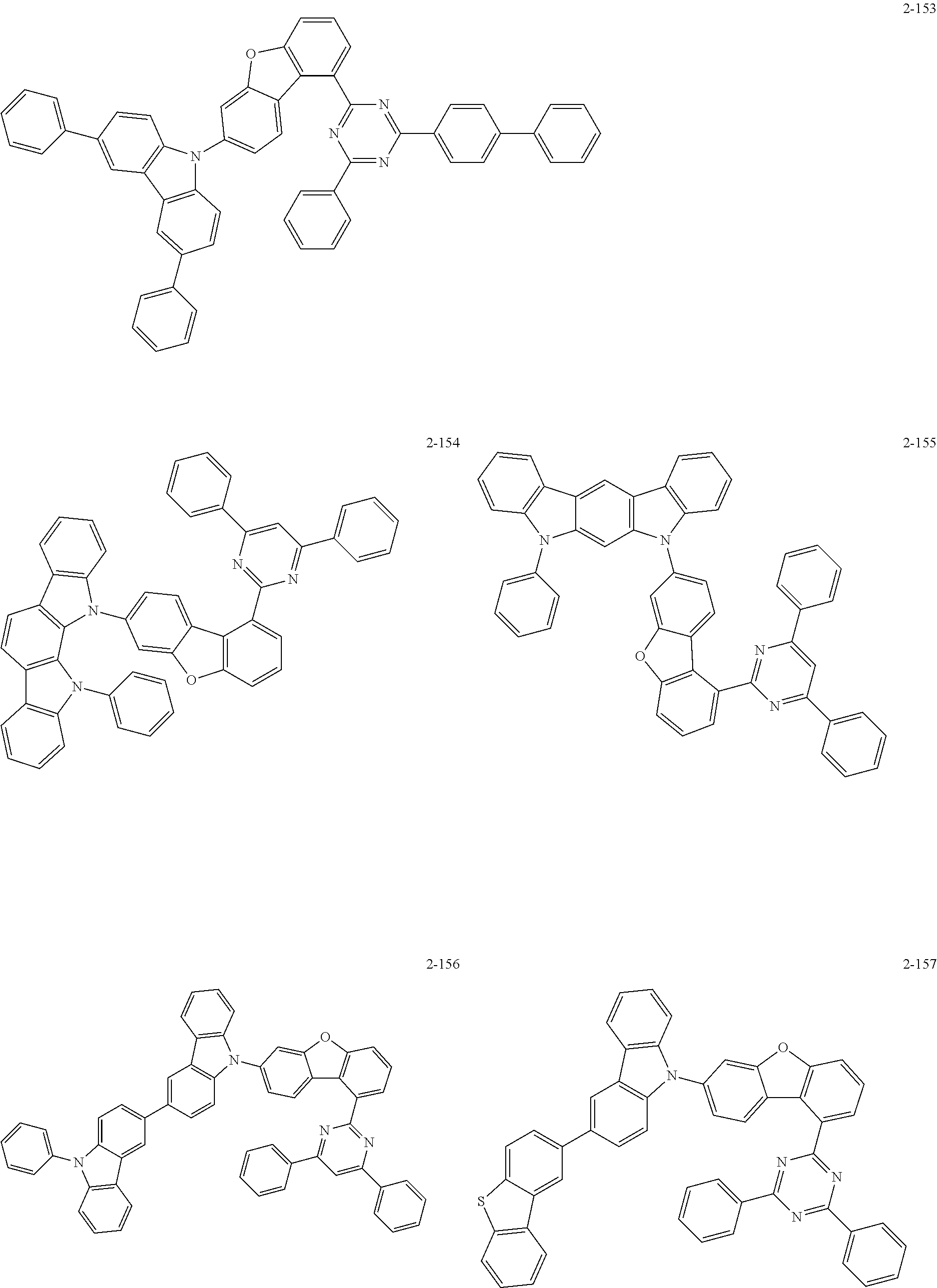

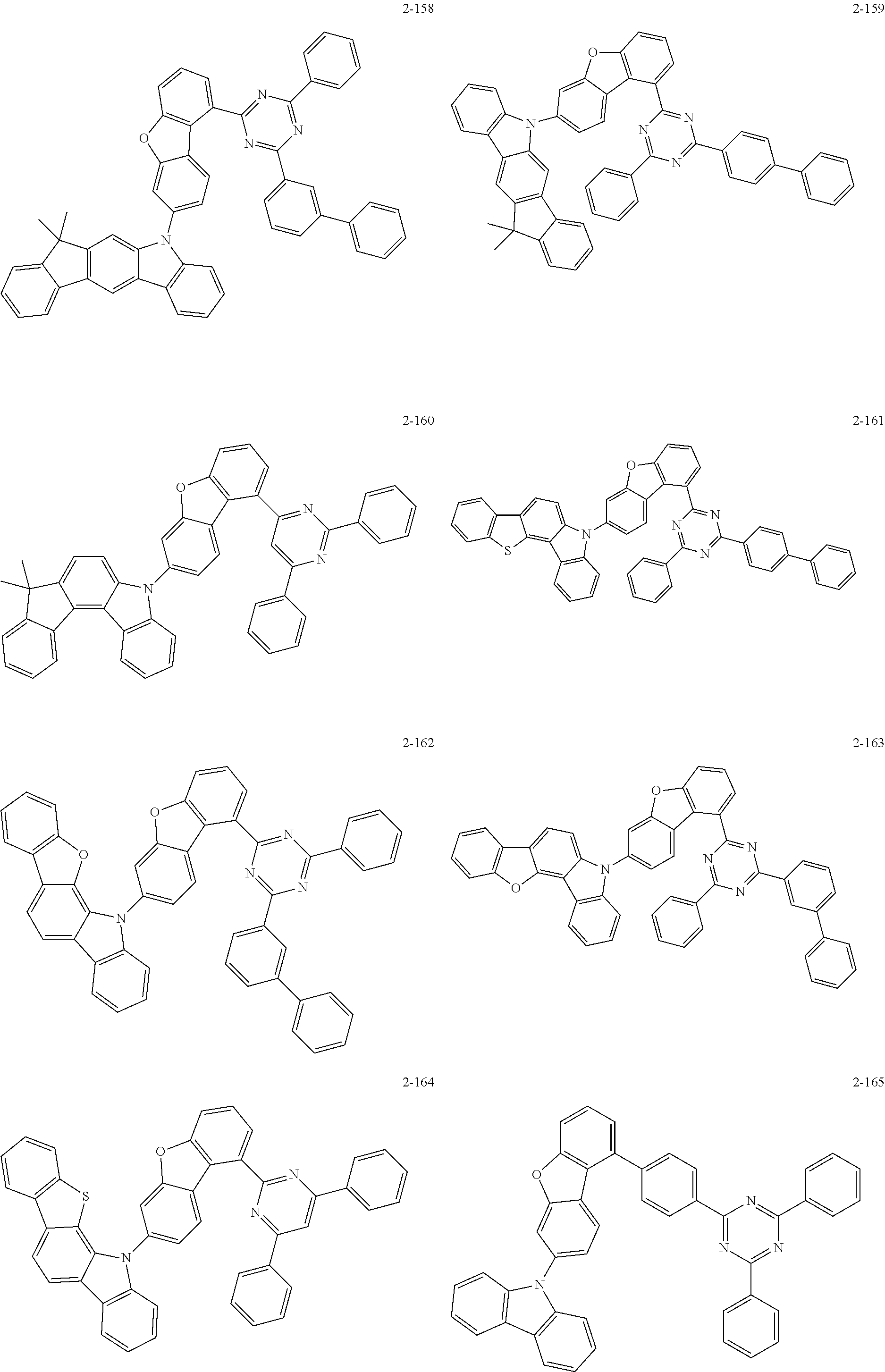

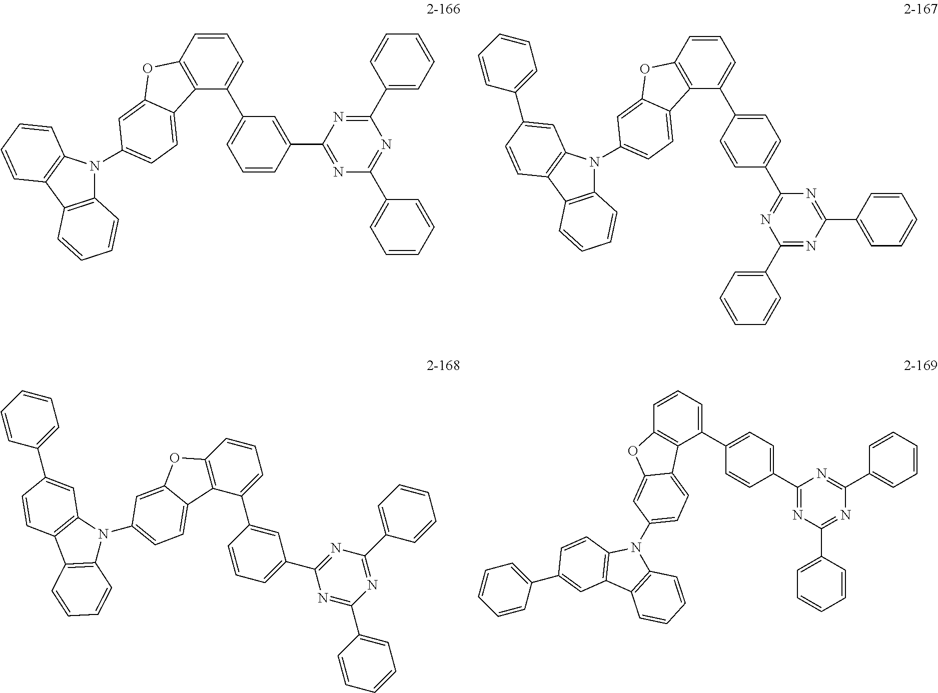 [SMILES: c1ccc(-c2nc(-c3ccccc3)nc(-c3cccc(-c4cccc5oc6cc(-n7c8ccccc8c8ccccc87)ccc6c45)c3)n2)cc1 ; c1ccc(-c2ccc3c4ccccc4n(-c4ccc5c(c4)oc4cccc(-c6ccc(-c7nc(-c8ccccc8)nc(-c8ccccc8)n7)cc6)c45)c3c2)cc1 ; c1ccc(-c2ccc3c4ccccc4n(-c4ccc5c(c4)oc4cccc(-c6cccc(-c7nc(-c8ccccc8)nc(-c8ccccc8)n7)c6)c45)c3c2)cc1 ; c1ccc(-c2ccc3c(c2)c2ccccc2n3-c2ccc3c(c2)oc2cccc(-c4ccc(-c5nc(-c6ccccc6)nc(-c6ccccc6)n5)cc4)c23)cc1]

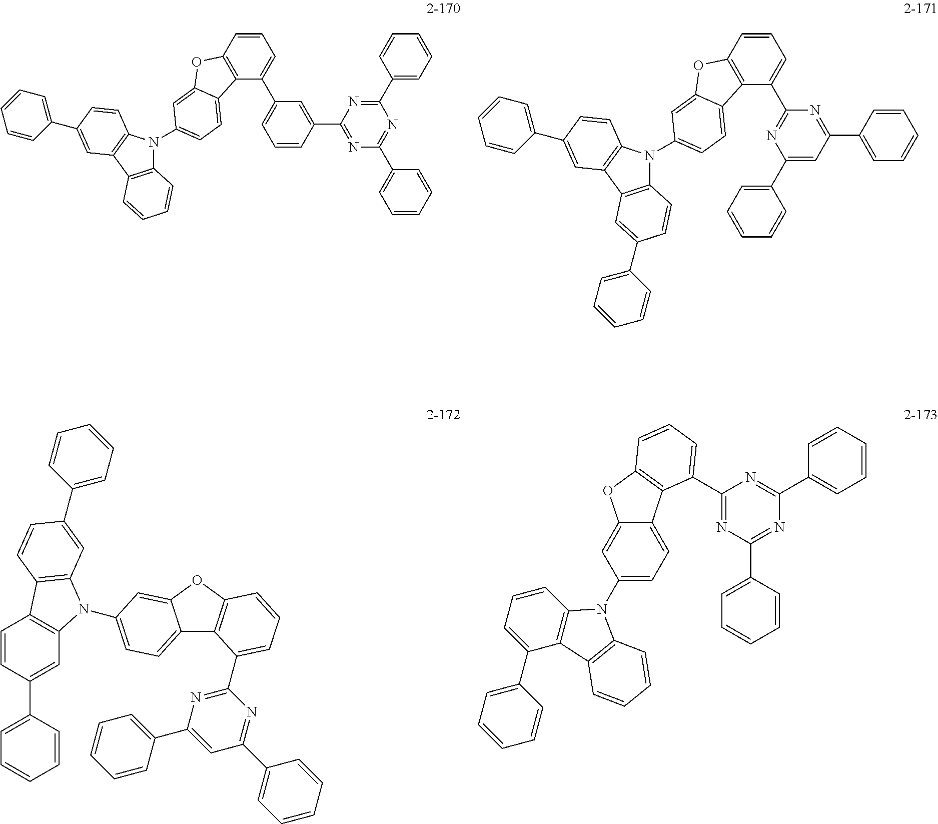 [SMILES: c1ccc(-c2ccc3c(c2)c2ccccc2n3-c2ccc3c(c2)oc2cccc(-c4cccc(-c5nc(-c6ccccc6)nc(-c6ccccc6)n5)c4)c23)cc1 ; c1ccc(-c2ccc3c(c2)c2cc(-c4ccccc4)ccc2n3-c2ccc3c(c2)oc2cccc(-c4nc(-c5ccccc5)cc(-c5ccccc5)n4)c23)cc1 ; c1ccc(-c2ccc3c4ccc(-c5ccccc5)cc4n(-c4ccc5c(c4)oc4cccc(-c6nc(-c7ccccc7)cc(-c7ccccc7)n6)c45)c3c2)cc1 ; c1ccc(-c2nc(-c3ccccc3)nc(-c3cccc4oc5cc(-n6c7ccccc7c7c(-c8ccccc8)cccc76)ccc5c34)n2)cc1]

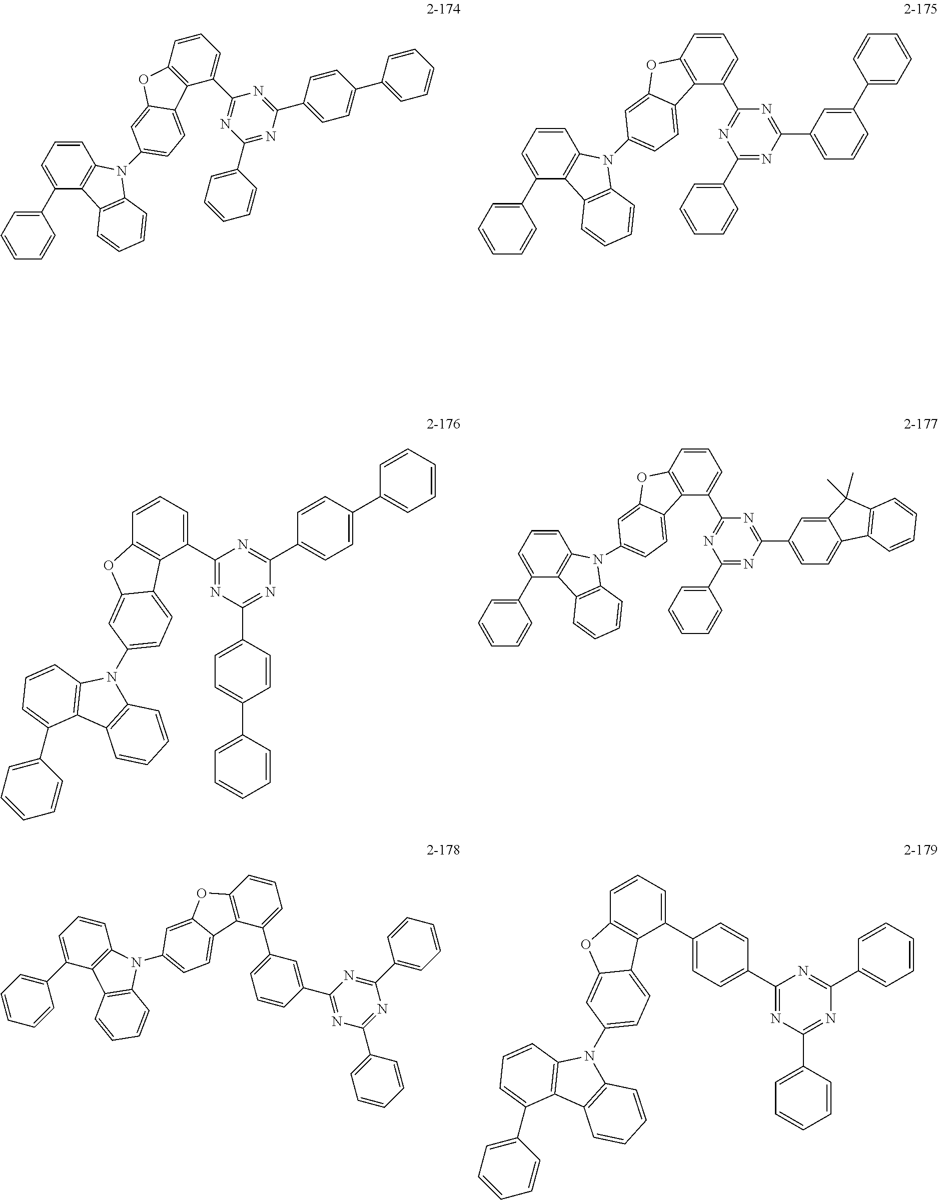

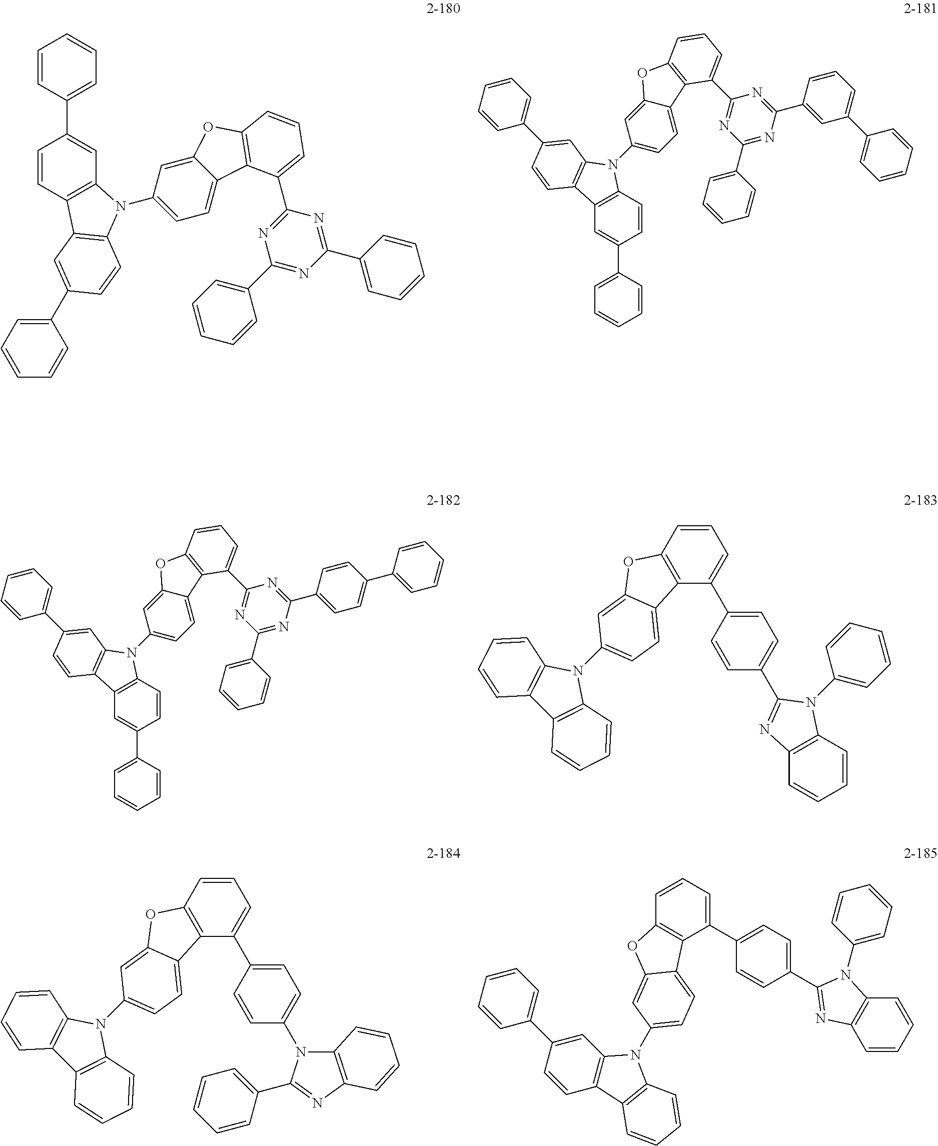

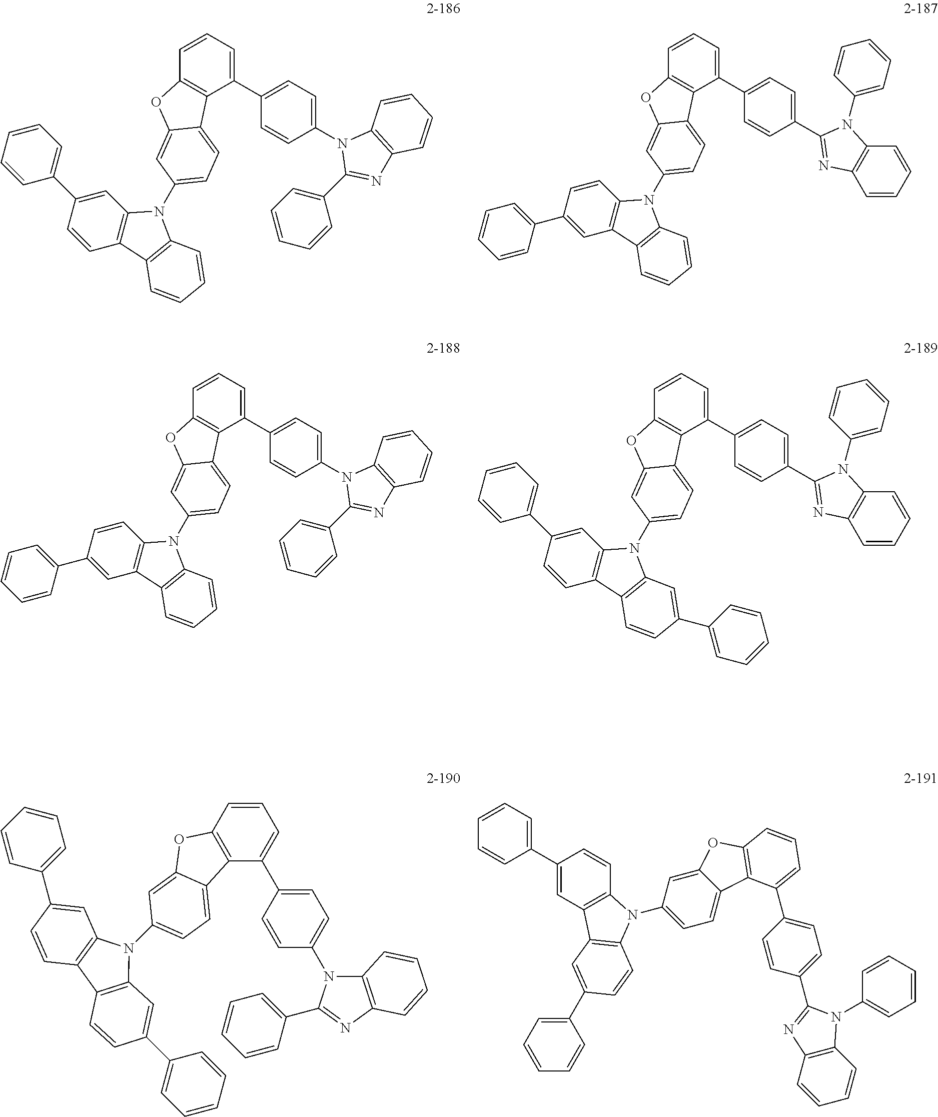

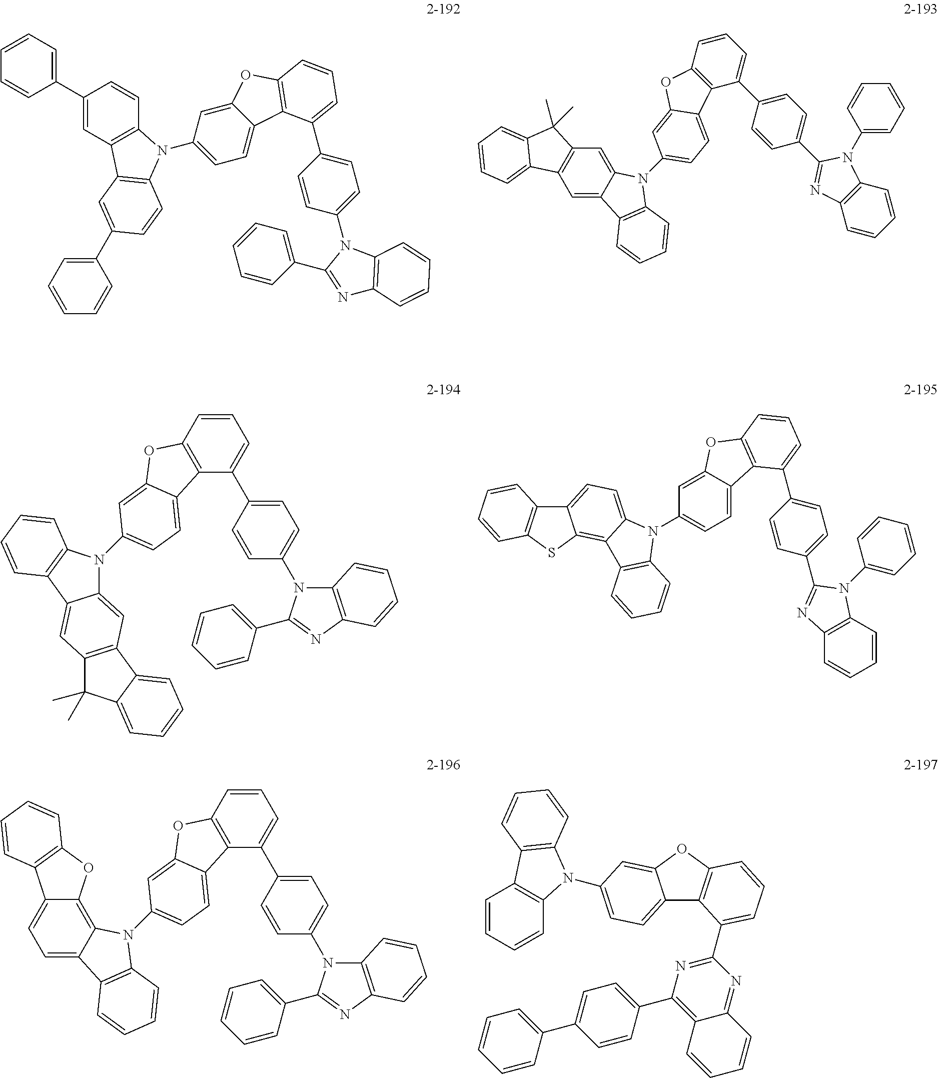

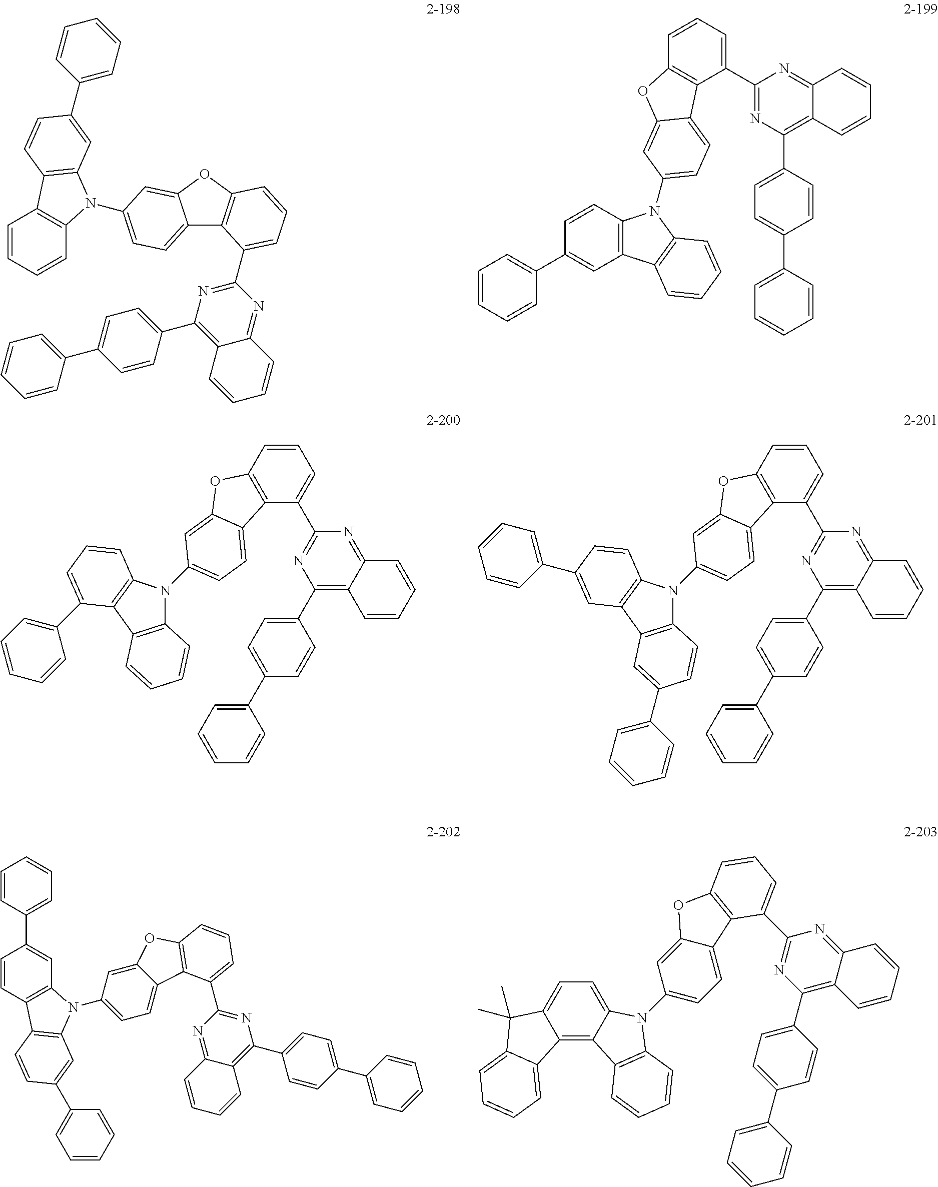

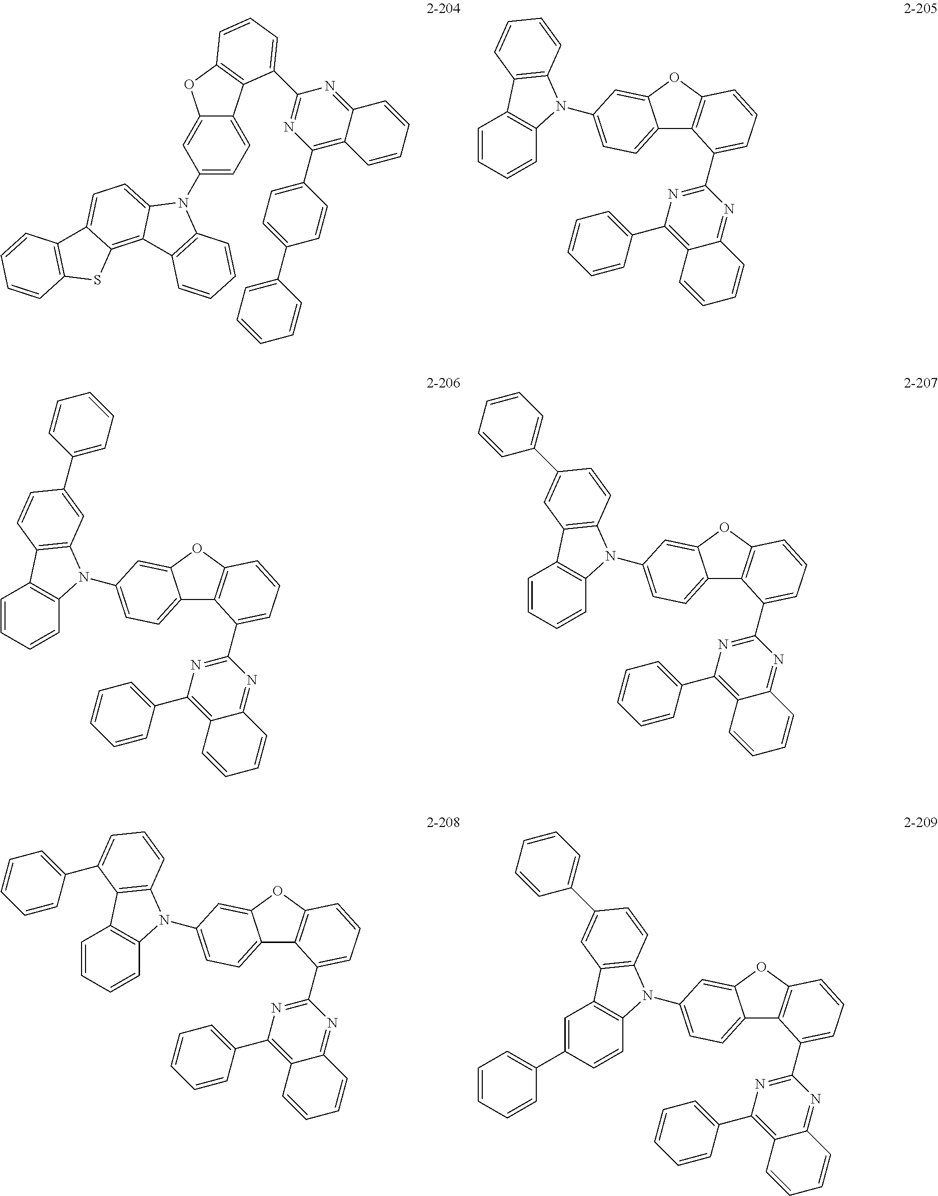

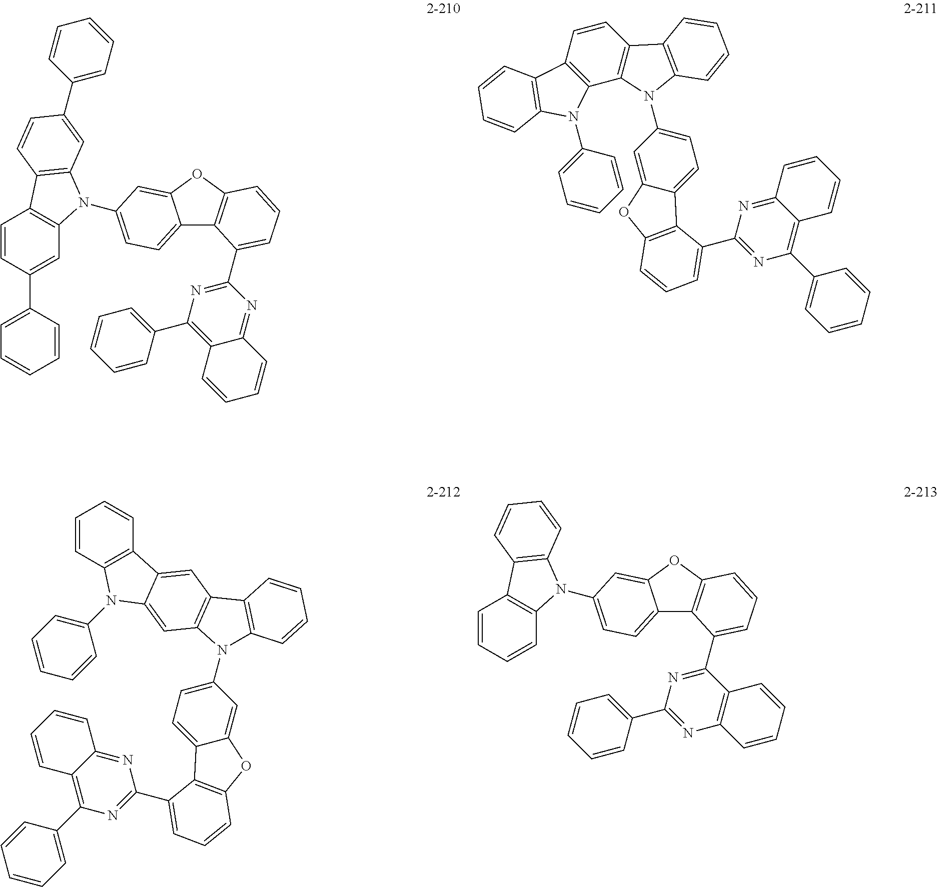 [SMILES: c1ccc(-c2ccc3c4ccc(-c5ccccc5)cc4n(-c4ccc5c(c4)oc4cccc(-c6nc(-c7ccccc7)c7ccccc7n6)c45)c3c2)cc1 ; c1ccc(-c2nc(-c3cccc4oc5cc(-n6c7ccccc7c7ccc8c9ccccc9n(-c9ccccc9)c8c76)ccc5c34)nc3ccccc23)cc1 ; c1ccc(-c2nc(-c3cccc4oc5cc(-n6c7ccccc7c7cc8c9ccccc9n(-c9ccccc9)c8cc76)ccc5c34)nc3ccccc23)cc1 ; c1ccc(-c2nc(-c3cccc4oc5cc(-n6c7ccccc7c7ccccc76)ccc5c34)c3ccccc3n2)cc1]

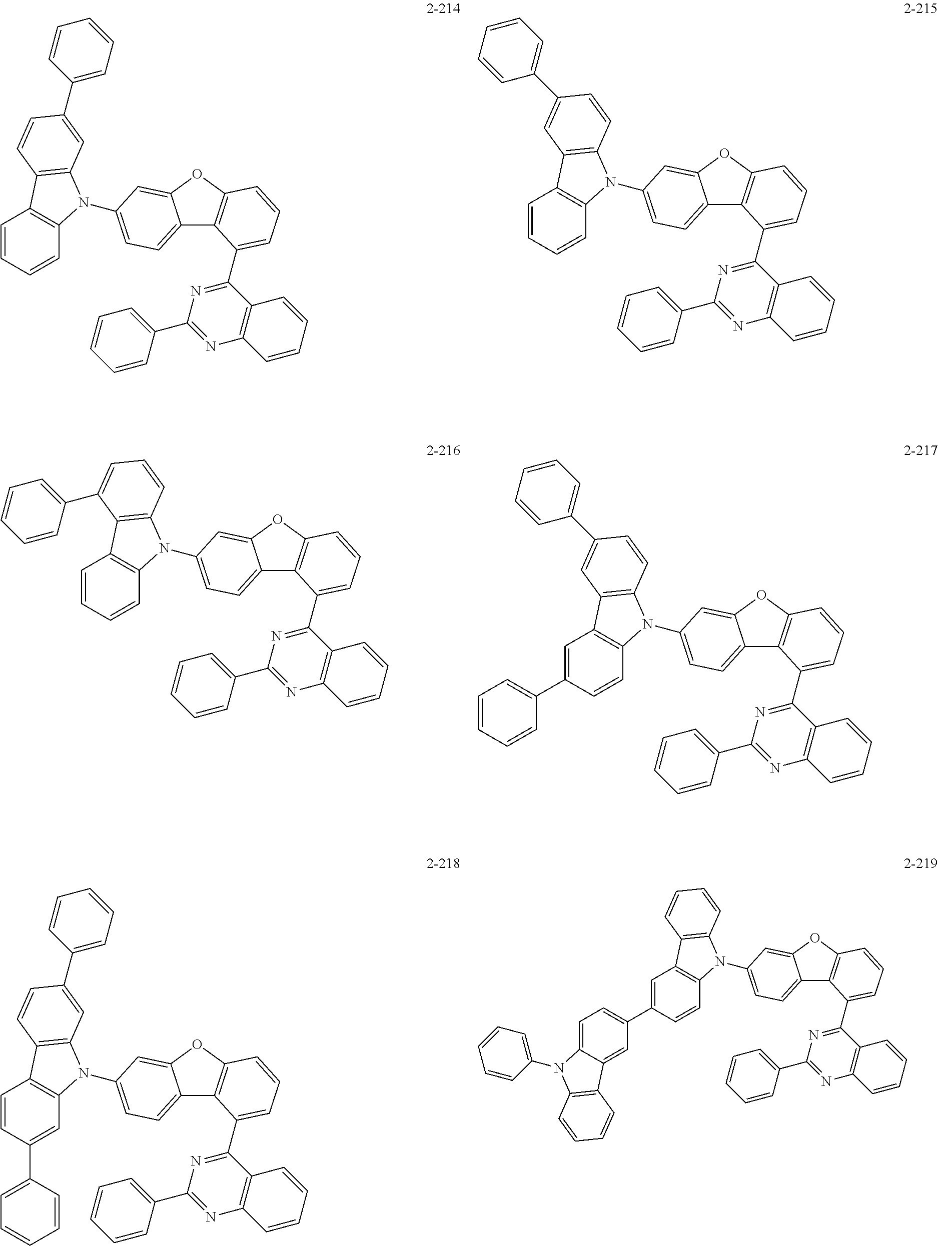

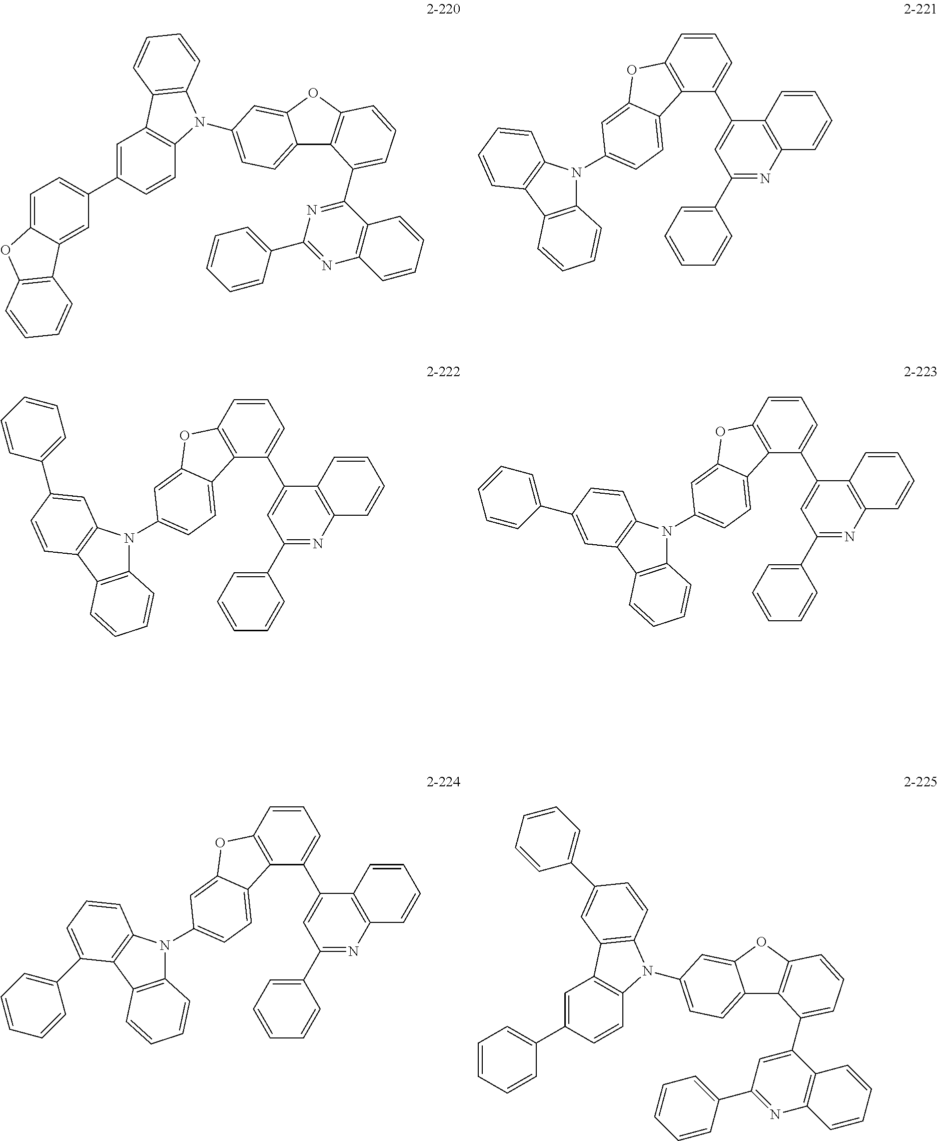

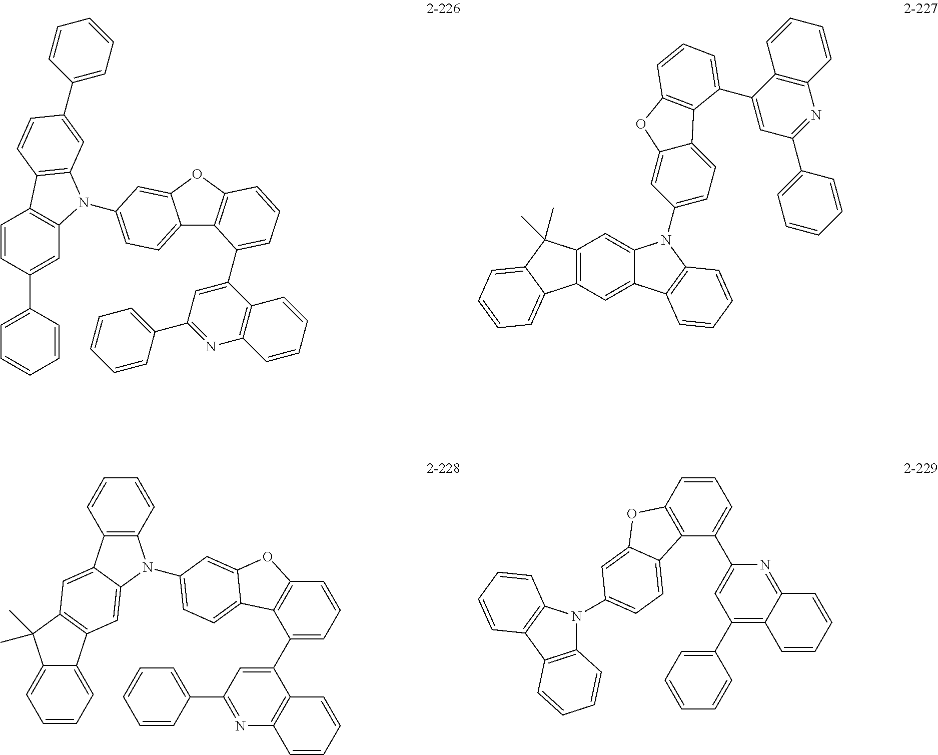 [SMILES: c1ccc(-c2ccc3c4ccc(-c5ccccc5)cc4n(-c4ccc5c(c4)oc4cccc(-c6cc(-c7ccccc7)nc7ccccc67)c45)c3c2)cc1 ; CC1(C)c2ccccc2-c2cc3c4ccccc4n(-c4ccc5c(c4)oc4cccc(-c6cc(-c7ccccc7)nc7ccccc67)c45)c3cc21 ; CC1(C)c2ccccc2-c2cc3c(cc21)c1ccccc1n3-c1ccc2c(c1)oc1cccc(-c3cc(-c4ccccc4)nc4ccccc34)c12 ; c1ccc(-c2cc(-c3cccc4oc5cc(-n6c7ccccc7c7ccccc76)ccc5c34)nc3ccccc23)cc1]

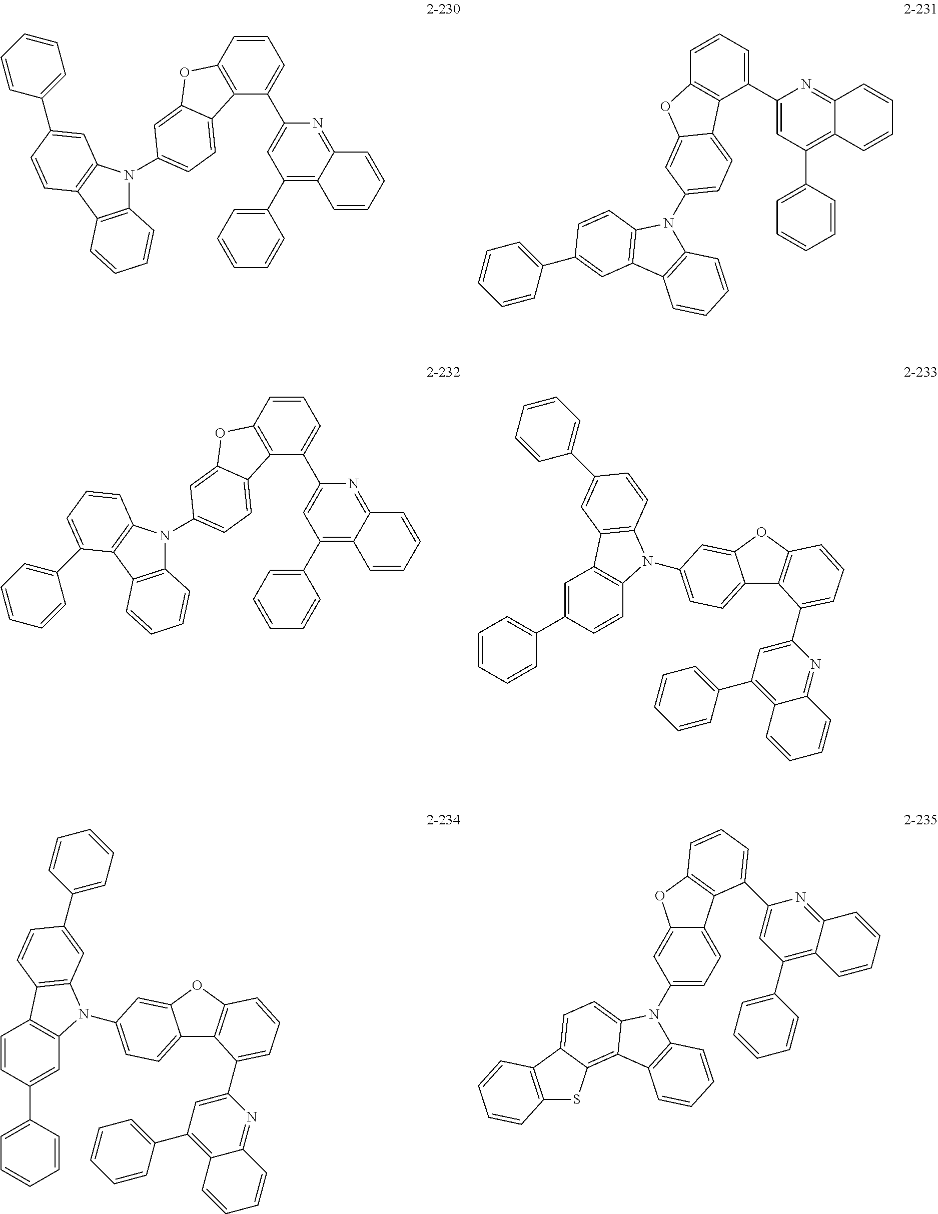

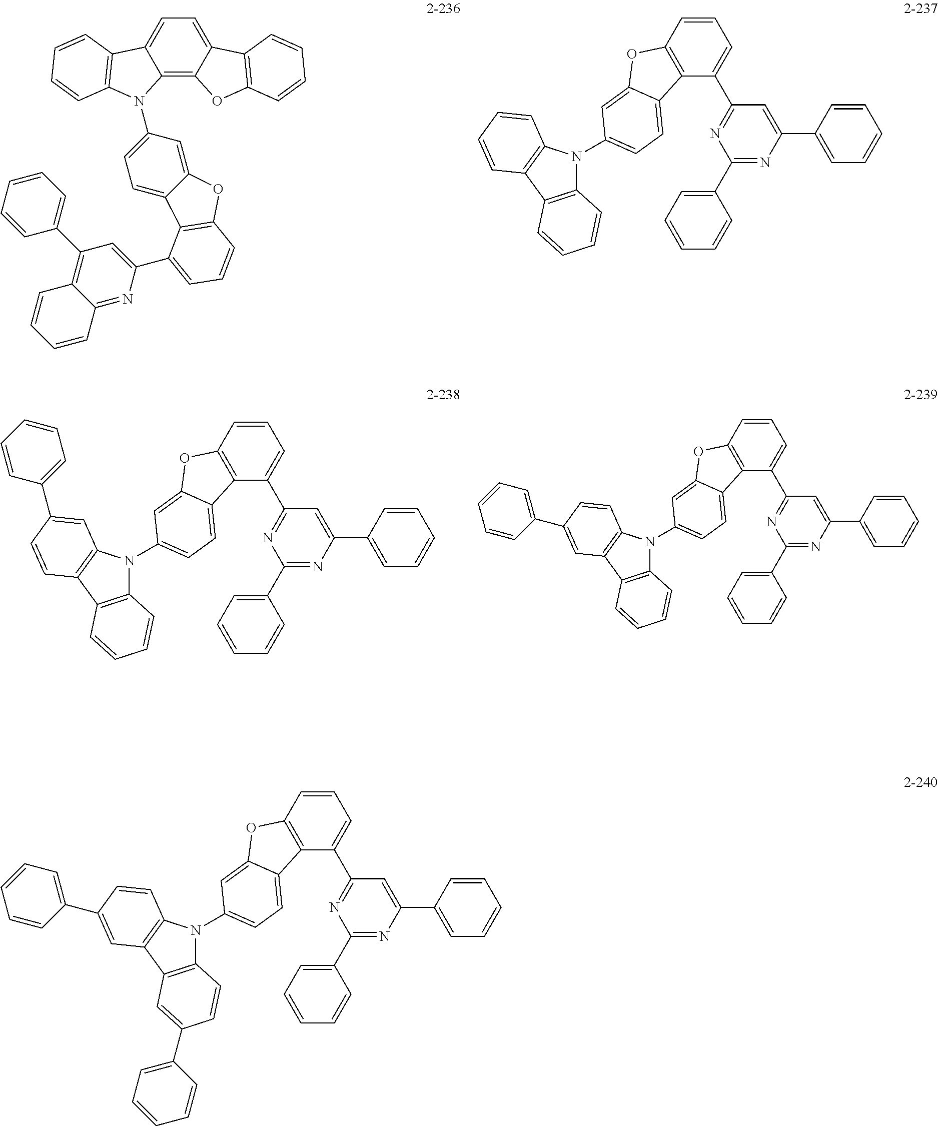 [SMILES: c1ccc(-c2cc(-c3cccc4oc5cc(-n6c7ccccc7c7ccc8c9ccccc9oc8c76)ccc5c34)nc3ccccc23)cc1 ; c1ccc(-c2cc(-c3cccc4oc5cc(-n6c7ccccc7c7ccccc76)ccc5c34)nc(-c3ccccc3)n2)cc1 ; c1ccc(-c2ccc3c4ccccc4n(-c4ccc5c(c4)oc4cccc(-c6cc(-c7ccccc7)nc(-c7ccccc7)n6)c45)c3c2)cc1 ; c1ccc(-c2ccc3c(c2)c2ccccc2n3-c2ccc3c(c2)oc2cccc(-c4cc(-c5ccccc5)nc(-c5ccccc5)n4)c23)cc1 ; c1ccc(-c2ccc3c(c2)c2cc(-c4ccccc4)ccc2n3-c2ccc3c(c2)oc2cccc(-c4cc(-c5ccccc5)nc(-c5ccccc5)n4)c23)cc1]

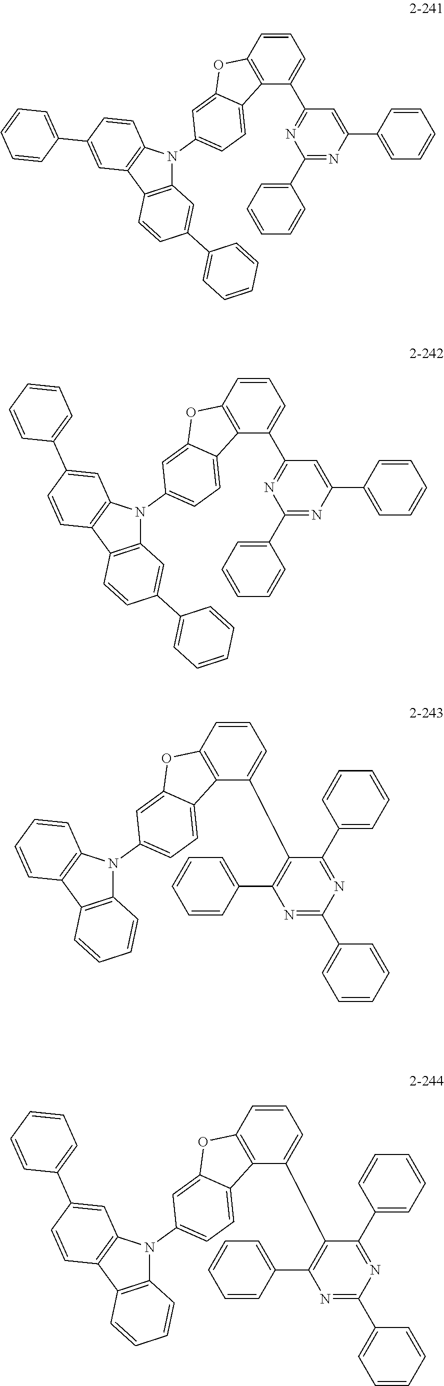 [SMILES: c1ccc(-c2ccc3c(c2)c2ccc(-c4ccccc4)cc2n3-c2ccc3c(c2)oc2cccc(-c4cc(-c5ccccc5)nc(-c5ccccc5)n4)c23)cc1 ; c1ccc(-c2ccc3c4ccc(-c5ccccc5)cc4n(-c4ccc5c(c4)oc4cccc(-c6cc(-c7ccccc7)nc(-c7ccccc7)n6)c45)c3c2)cc1 ; c1ccc(-c2nc(-c3ccccc3)c(-c3cccc4oc5cc(-n6c7ccccc7c7ccccc76)ccc5c34)c(-c3ccccc3)n2)cc1 ; c1ccc(-c2ccc3c4ccccc4n(-c4ccc5c(c4)oc4cccc(-c6c(-c7ccccc7)nc(-c7ccccc7)nc6-c6ccccc6)c45)c3c2)cc1]

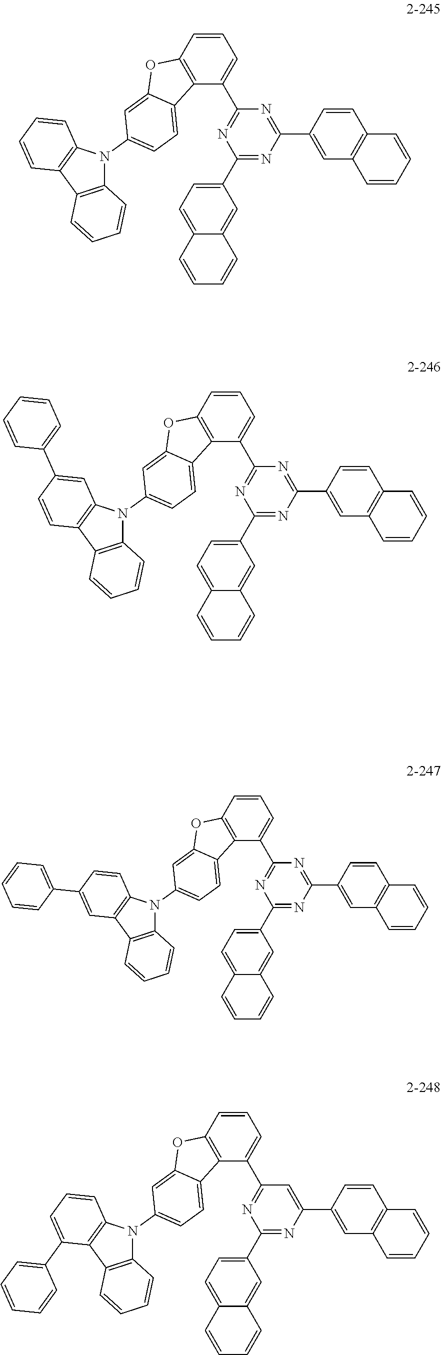 [SMILES: c1ccc2cc(-c3nc(-c4ccc5ccccc5c4)nc(-c4cccc5oc6cc(-n7c8ccccc8c8ccccc87)ccc6c45)n3)ccc2c1 ; c1ccc(-c2ccc3c4ccccc4n(-c4ccc5c(c4)oc4cccc(-c6nc(-c7ccc8ccccc8c7)nc(-c7ccc8ccccc8c7)n6)c45)c3c2)cc1 ; c1ccc(-c2ccc3c(c2)c2ccccc2n3-c2ccc3c(c2)oc2cccc(-c4nc(-c5ccc6ccccc6c5)nc(-c5ccc6ccccc6c5)n4)c23)cc1 ; c1ccc(-c2cccc3c2c2ccccc2n3-c2ccc3c(c2)oc2cccc(-c4cc(-c5ccc6ccccc6c5)nc(-c5ccc6ccccc6c5)n4)c23)cc1]

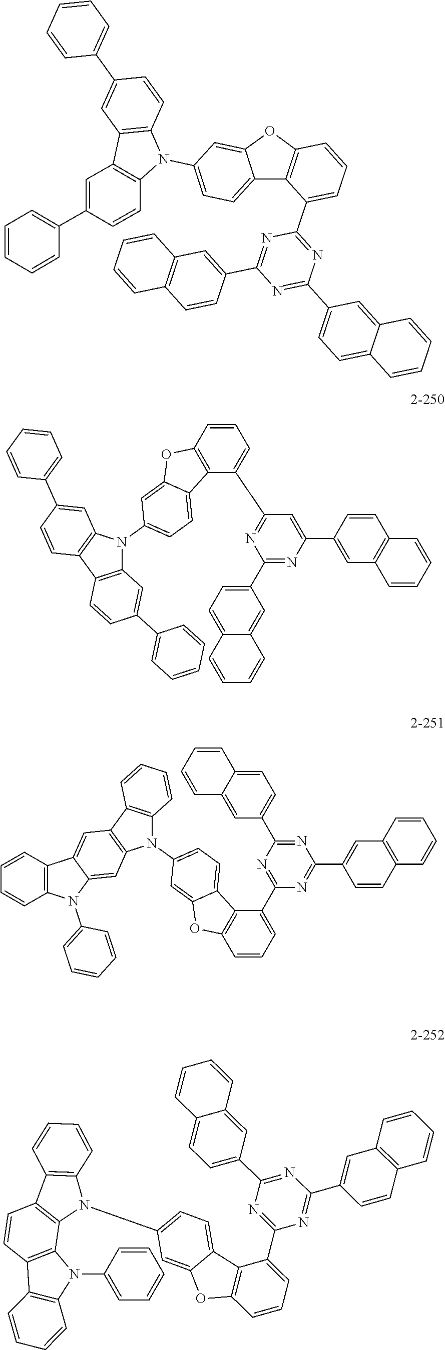 [SMILES: c1ccc(-c2ccc3c(c2)c2cc(-c4ccccc4)ccc2n3-c2ccc3c(c2)oc2cccc(-c4nc(-c5ccc6ccccc6c5)nc(-c5ccc6ccccc6c5)n4)c23)cc1 ; c1ccc(-c2ccc3c4ccc(-c5ccccc5)cc4n(-c4ccc5c(c4)oc4cccc(-c6cc(-c7ccc8ccccc8c7)nc(-c7ccc8ccccc8c7)n6)c45)c3c2)cc1 ; c1ccc(-n2c3ccccc3c3cc4c5ccccc5n(-c5ccc6c(c5)oc5cccc(-c7nc(-c8ccc9ccccc9c8)nc(-c8ccc9ccccc9c8)n7)c56)c4cc32)cc1 ; c1ccc(-n2c3ccccc3c3ccc4c5ccccc5n(-c5ccc6c(c5)oc5cccc(-c7nc(-c8ccc9ccccc9c8)nc(-c8ccc9ccccc9c8)n7)c56)c4c32)cc1]

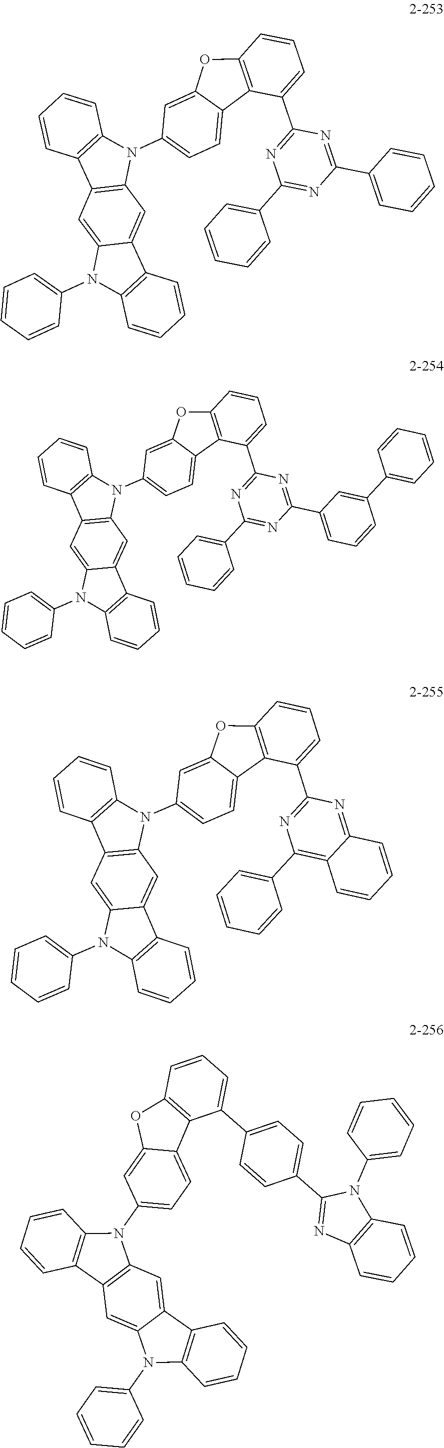 [SMILES: c1ccc(-c2nc(-c3ccccc3)nc(-c3cccc4oc5cc(-n6c7ccccc7c7cc8c(cc76)c6ccccc6n8-c6ccccc6)ccc5c34)n2)cc1 ; c1ccc(-c2cccc(-c3nc(-c4ccccc4)nc(-c4cccc5oc6cc(-n7c8ccccc8c8cc9c(cc87)c7ccccc7n9-c7ccccc7)ccc6c45)n3)c2)cc1 ; c1ccc(-c2nc(-c3cccc4oc5cc(-n6c7ccccc7c7cc8c(cc76)c6ccccc6n8-c6ccccc6)ccc5c34)nc3ccccc23)cc1 ; c1ccc(-n2c(-c3ccc(-c4cccc5oc6cc(-n7c8ccccc8c8cc9c(cc87)c7ccccc7n9-c7ccccc7)ccc6c45)cc3)nc3ccccc32)cc1]

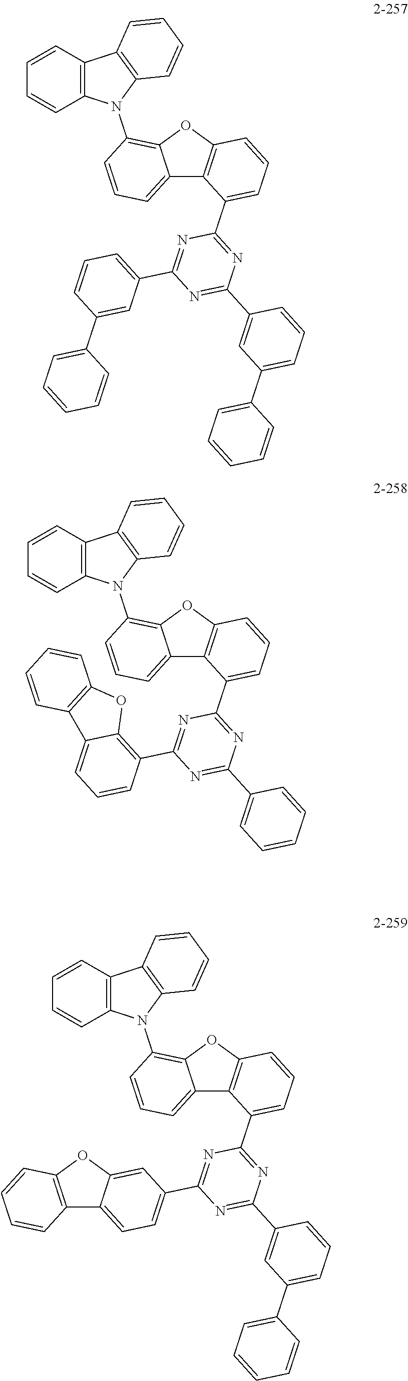 [SMILES: c1ccc(-c2cccc(-c3nc(-c4cccc(-c5ccccc5)c4)nc(-c4cccc5oc6c(-n7c8ccccc8c8ccccc87)cccc6c45)n3)c2)cc1 ; c1ccc(-c2nc(-c3cccc4c3oc3ccccc34)nc(-c3cccc4oc5c(-n6c7ccccc7c7ccccc76)cccc5c34)n2)cc1 ; c1ccc(-c2cccc(-c3nc(-c4ccc5c(c4)oc4ccccc45)nc(-c4cccc5oc6c(-n7c8ccccc8c8ccccc87)cccc6c45)n3)c2)cc1]

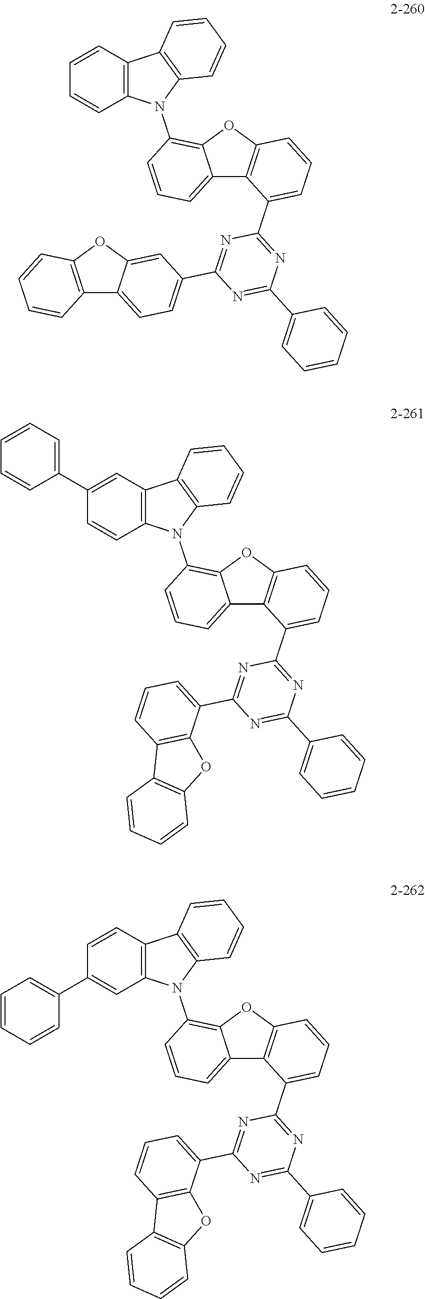 [SMILES: c1ccc(-c2nc(-c3ccc4c(c3)oc3ccccc34)nc(-c3cccc4oc5c(-n6c7ccccc7c7ccccc76)cccc5c34)n2)cc1 ; c1ccc(-c2ccc3c(c2)c2ccccc2n3-c2cccc3c2oc2cccc(-c4nc(-c5ccccc5)nc(-c5cccc6c5oc5ccccc56)n4)c23)cc1 ; c1ccc(-c2ccc3c4ccccc4n(-c4cccc5c4oc4cccc(-c6nc(-c7ccccc7)nc(-c7cccc8c7oc7ccccc78)n6)c45)c3c2)cc1]

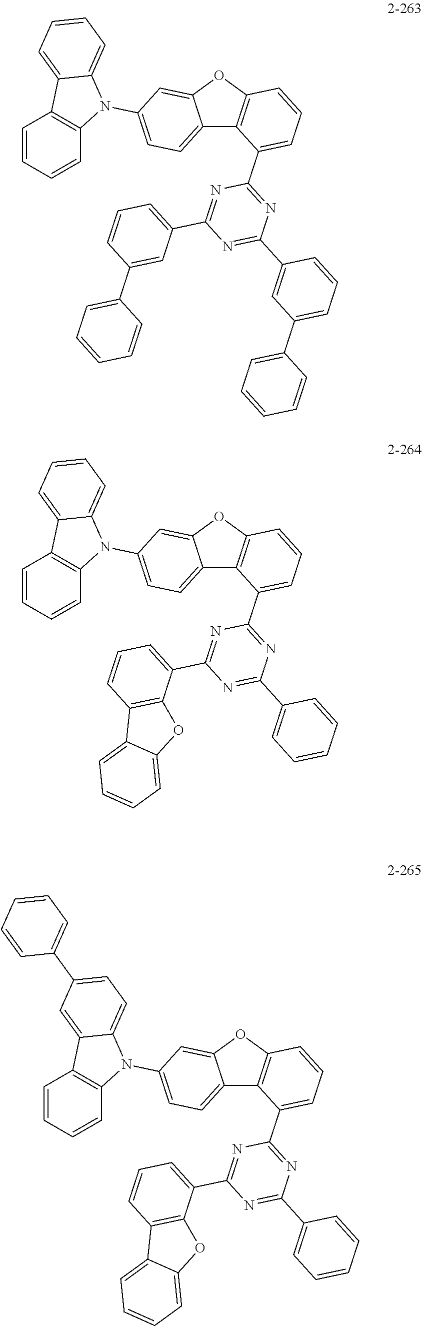 [SMILES: c1ccc(-c2cccc(-c3nc(-c4cccc(-c5ccccc5)c4)nc(-c4cccc5oc6cc(-n7c8ccccc8c8ccccc87)ccc6c45)n3)c2)cc1 ; c1ccc(-c2nc(-c3cccc4c3oc3ccccc34)nc(-c3cccc4oc5cc(-n6c7ccccc7c7ccccc76)ccc5c34)n2)cc1 ; c1ccc(-c2ccc3c(c2)c2ccccc2n3-c2ccc3c(c2)oc2cccc(-c4nc(-c5ccccc5)nc(-c5cccc6c5oc5ccccc56)n4)c23)cc1]

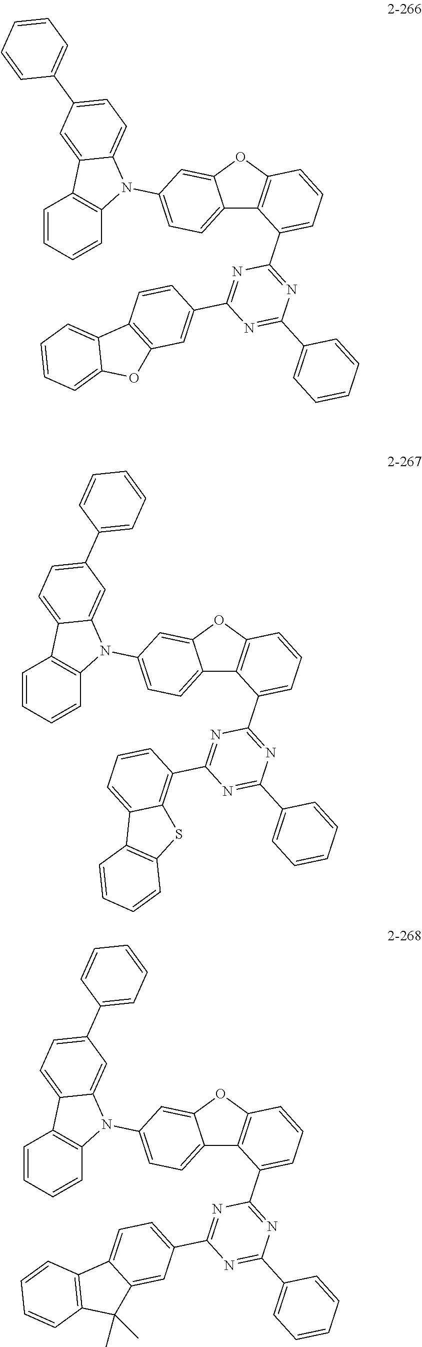

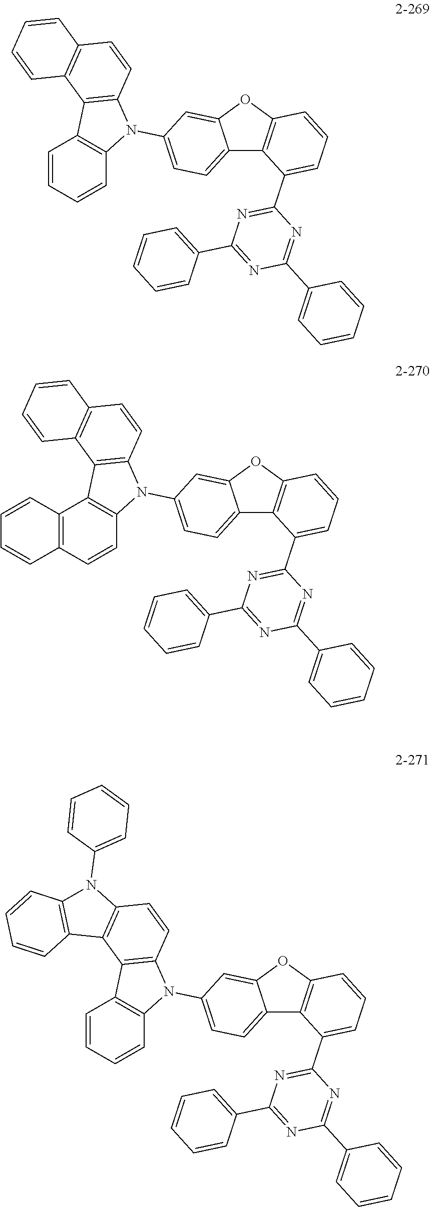 [SMILES: c1ccc(-c2nc(-c3ccccc3)nc(-c3cccc4oc5cc(-n6c7ccccc7c7c8ccccc8ccc76)ccc5c34)n2)cc1 ; c1ccc(-c2nc(-c3ccccc3)nc(-c3cccc4oc5cc(-n6c7ccc8ccccc8c7c7c8ccccc8ccc76)ccc5c34)n2)cc1 ; c1ccc(-c2nc(-c3ccccc3)nc(-c3cccc4oc5cc(-n6c7ccccc7c7c8c9ccccc9n(-c9ccccc9)c8ccc76)ccc5c34)n2)cc1]

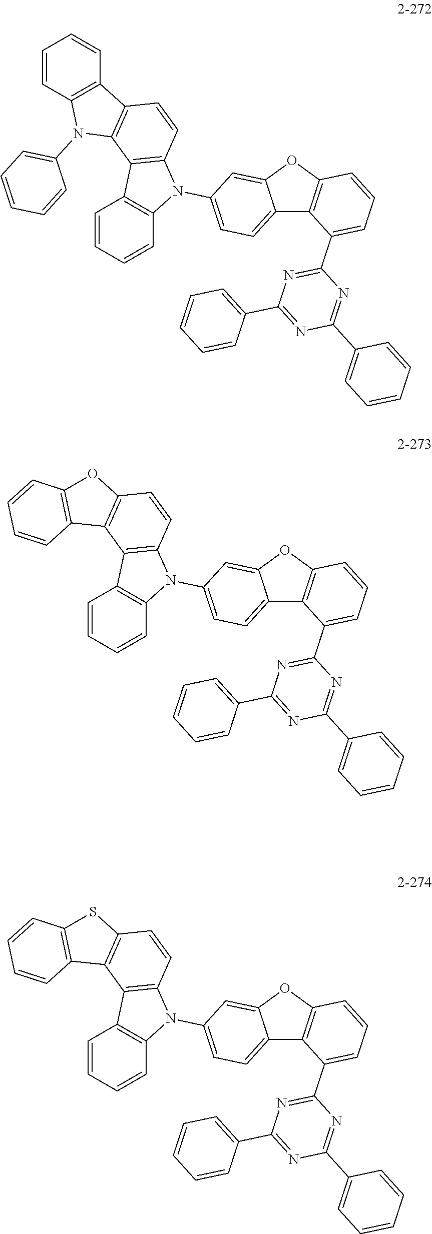 [SMILES: c1ccc(-c2nc(-c3ccccc3)nc(-c3cccc4oc5cc(-n6c7ccccc7c7c6ccc6c8ccccc8n(-c8ccccc8)c67)ccc5c34)n2)cc1 ; c1ccc(-c2nc(-c3ccccc3)nc(-c3cccc4oc5cc(-n6c7ccccc7c7c8c(ccc76)oc6ccccc68)ccc5c34)n2)cc1 ; c1ccc(-c2nc(-c3ccccc3)nc(-c3cccc4oc5cc(-n6c7ccccc7c7c8c(ccc76)sc6ccccc68)ccc5c34)n2)cc1]

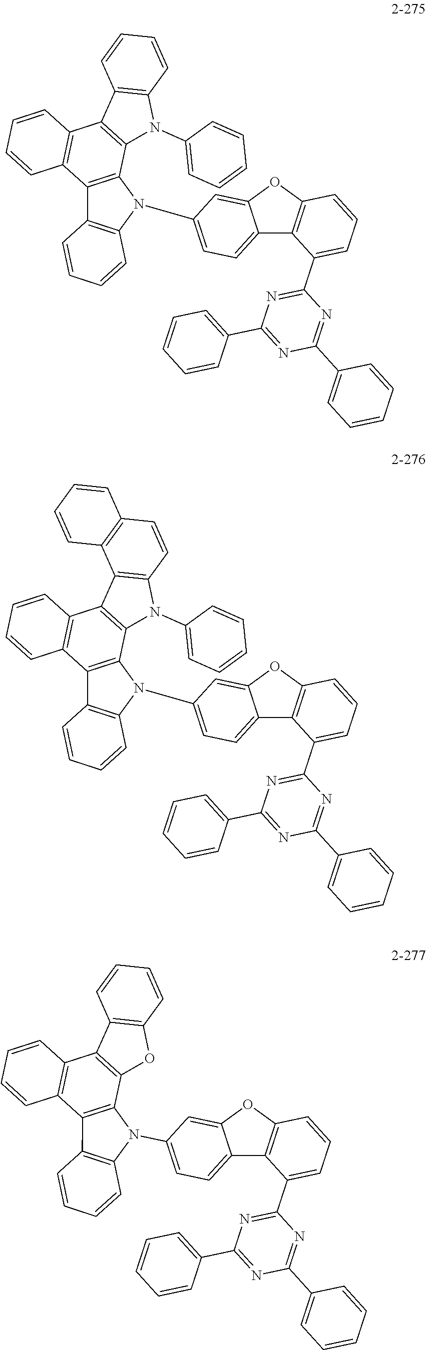 [SMILES: c1ccc(-c2nc(-c3ccccc3)nc(-c3cccc4oc5cc(-n6c7ccccc7c7c8ccccc8c8c9ccccc9n(-c9ccccc9)c8c76)ccc5c34)n2)cc1 ; c1ccc(-c2nc(-c3ccccc3)nc(-c3cccc4oc5cc(-n6c7ccccc7c7c8ccccc8c8c9c%10ccccc%10ccc9n(-c9ccccc9)c8c76)ccc5c34)n2)cc1 ; c1ccc(-c2nc(-c3ccccc3)nc(-c3cccc4oc5cc(-n6c7ccccc7c7c8ccccc8c8c9ccccc9oc8c76)ccc5c34)n2)cc1]

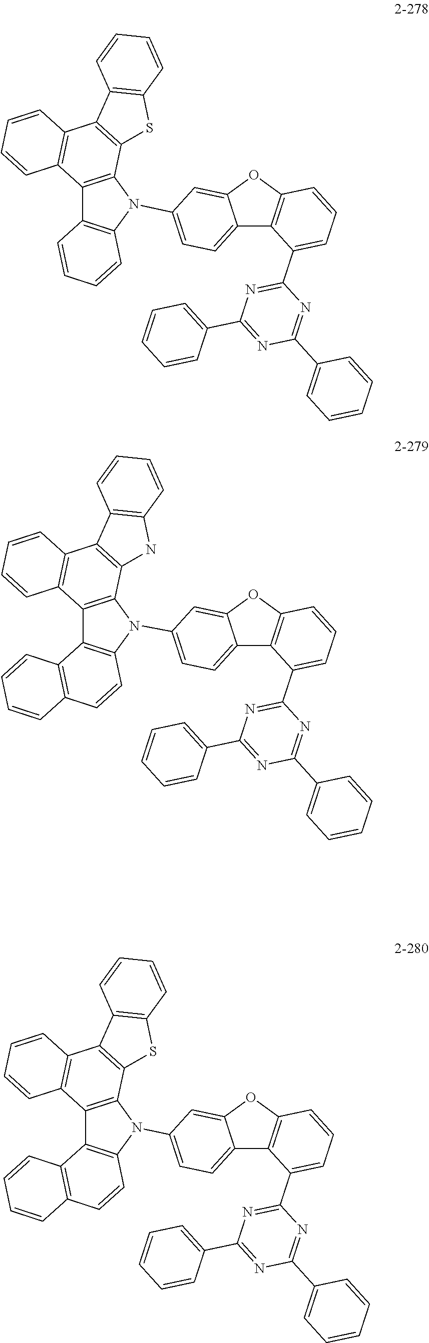 [SMILES: c1ccc(-c2nc(-c3ccccc3)nc(-c3cccc4oc5cc(-n6c7ccccc7c7c8ccccc8c8c9ccccc9sc8c76)ccc5c34)n2)cc1 ; c1ccc(-c2nc(-c3ccccc3)nc(-c3cccc4oc5cc(-n6c7ccc8ccccc8c7c7c8ccccc8c8c9ccccc9[nH]c8c76)ccc5c34)n2)cc1 ; c1ccc(-c2nc(-c3ccccc3)nc(-c3cccc4oc5cc(-n6c7ccc8ccccc8c7c7c8ccccc8c8c9ccccc9sc8c76)ccc5c34)n2)cc1]

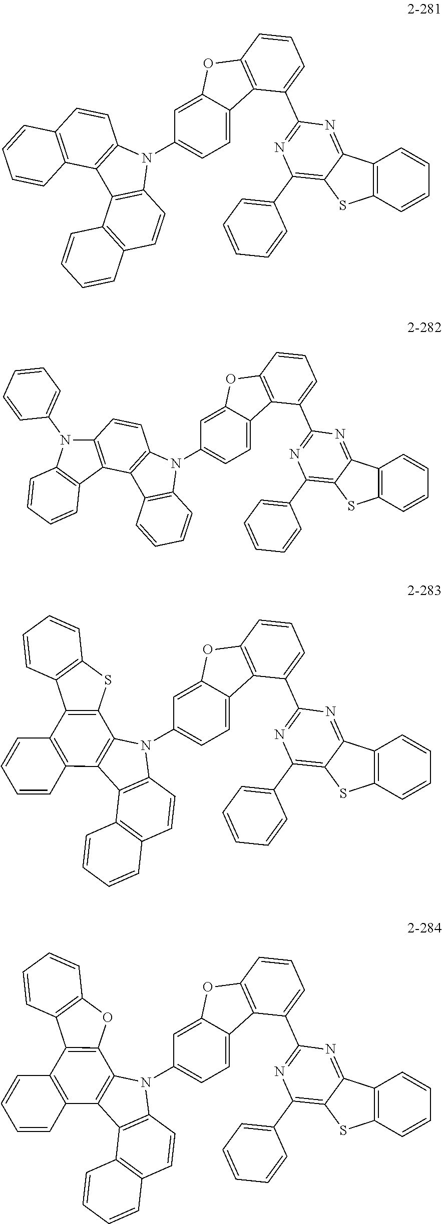 [SMILES: c1ccc(-c2nc(-c3cccc4oc5cc(-n6c7ccc8ccccc8c7c7c8ccccc8ccc76)ccc5c34)nc3c2sc2ccccc23)cc1 ; c1ccc(-c2nc(-c3cccc4oc5cc(-n6c7ccccc7c7c8c9ccccc9n(-c9ccccc9)c8ccc76)ccc5c34)nc3c2sc2ccccc23)cc1 ; c1ccc(-c2nc(-c3cccc4oc5cc(-n6c7ccc8ccccc8c7c7c8ccccc8c8c9ccccc9sc8c76)ccc5c34)nc3c2sc2ccccc23)cc1 ; c1ccc(-c2nc(-c3cccc4oc5cc(-n6c7ccc8ccccc8c7c7c8ccccc8c8c9ccccc9oc8c76)ccc5c34)nc3c2sc2ccccc23)cc1]

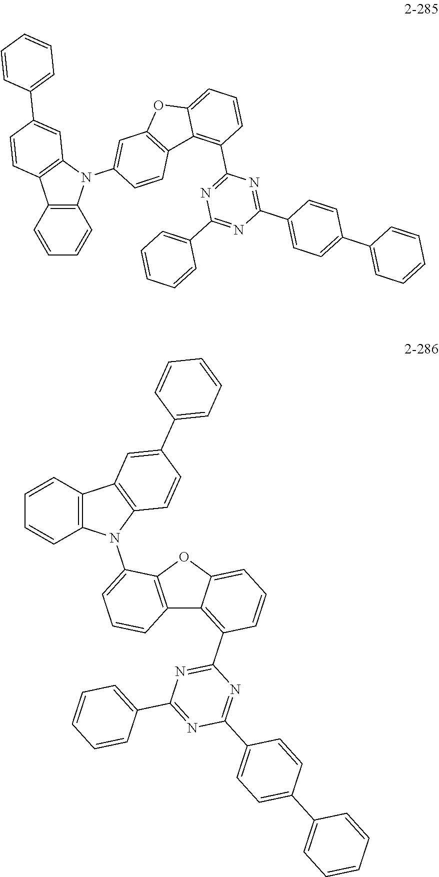 [SMILES: c1ccc(-c2ccc(-c3nc(-c4ccccc4)nc(-c4cccc5oc6cc(-n7c8ccccc8c8ccc(-c9ccccc9)cc87)ccc6c45)n3)cc2)cc1 ; c1ccc(-c2ccc(-c3nc(-c4ccccc4)nc(-c4cccc5oc6c(-n7c8ccccc8c8cc(-c9ccccc9)ccc87)cccc6c45)n3)cc2)cc1]

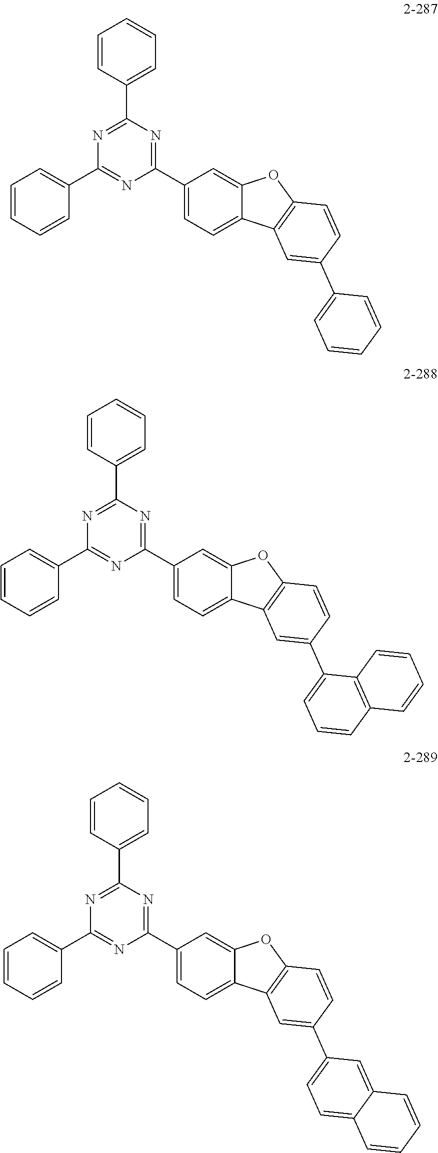 [SMILES: c1ccc(-c2ccc3oc4cc(-c5nc(-c6ccccc6)nc(-c6ccccc6)n5)ccc4c3c2)cc1 ; c1ccc(-c2nc(-c3ccccc3)nc(-c3ccc4c(c3)oc3ccc(-c5cccc6ccccc56)cc34)n2)cc1 ; c1ccc(-c2nc(-c3ccccc3)nc(-c3ccc4c(c3)oc3ccc(-c5ccc6ccccc6c5)cc34)n2)cc1]

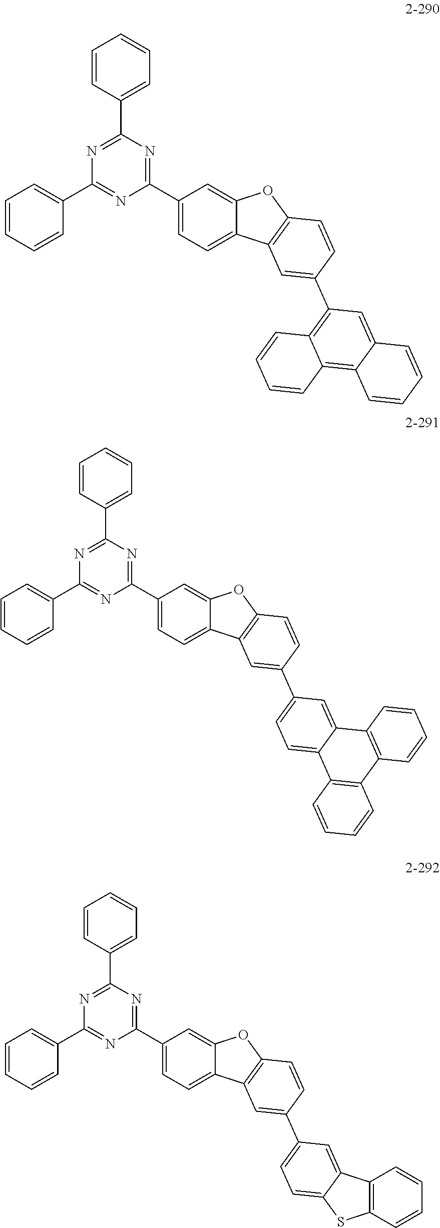 [SMILES: c1ccc(-c2nc(-c3ccccc3)nc(-c3ccc4c(c3)oc3ccc(-c5cc6ccccc6c6ccccc56)cc34)n2)cc1 ; c1ccc(-c2nc(-c3ccccc3)nc(-c3ccc4c(c3)oc3ccc(-c5ccc6c7ccccc7c7ccccc7c6c5)cc34)n2)cc1 ; c1ccc(-c2nc(-c3ccccc3)nc(-c3ccc4c(c3)oc3ccc(-c5ccc6sc7ccccc7c6c5)cc34)n2)cc1]

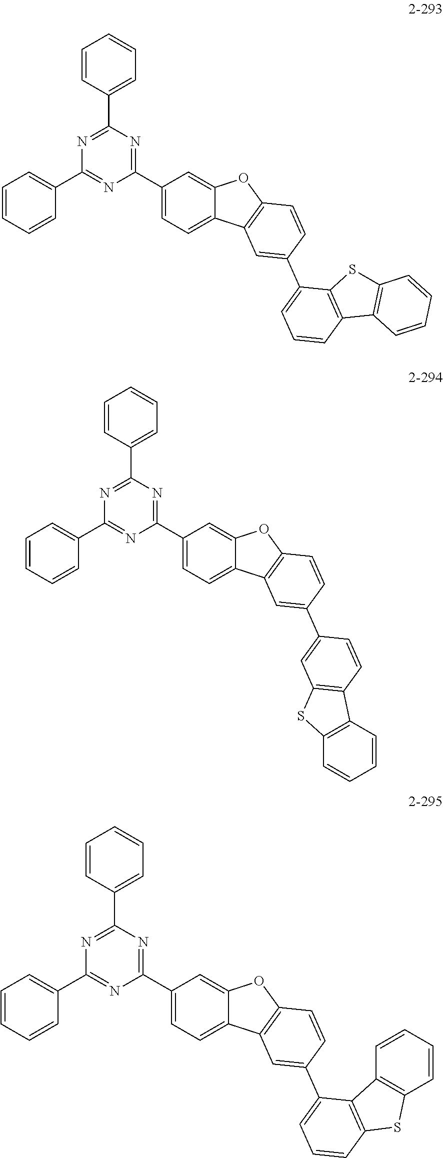 [SMILES: c1ccc(-c2nc(-c3ccccc3)nc(-c3ccc4c(c3)oc3ccc(-c5cccc6c5sc5ccccc56)cc34)n2)cc1 ; c1ccc(-c2nc(-c3ccccc3)nc(-c3ccc4c(c3)oc3ccc(-c5ccc6c(c5)sc5ccccc56)cc34)n2)cc1 ; c1ccc(-c2nc(-c3ccccc3)nc(-c3ccc4c(c3)oc3ccc(-c5cccc6sc7ccccc7c56)cc34)n2)cc1]

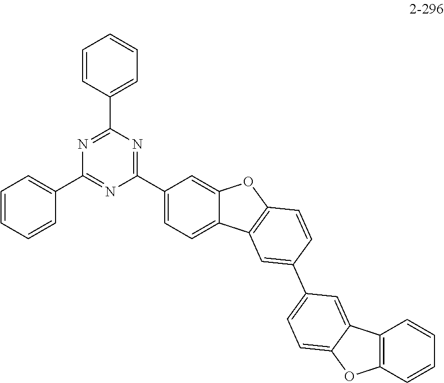 [SMILES: c1ccc(-c2nc(-c3ccccc3)nc(-c3ccc4c(c3)oc3ccc(-c5ccc6oc7ccccc7c6c5)cc34)n2)cc1]

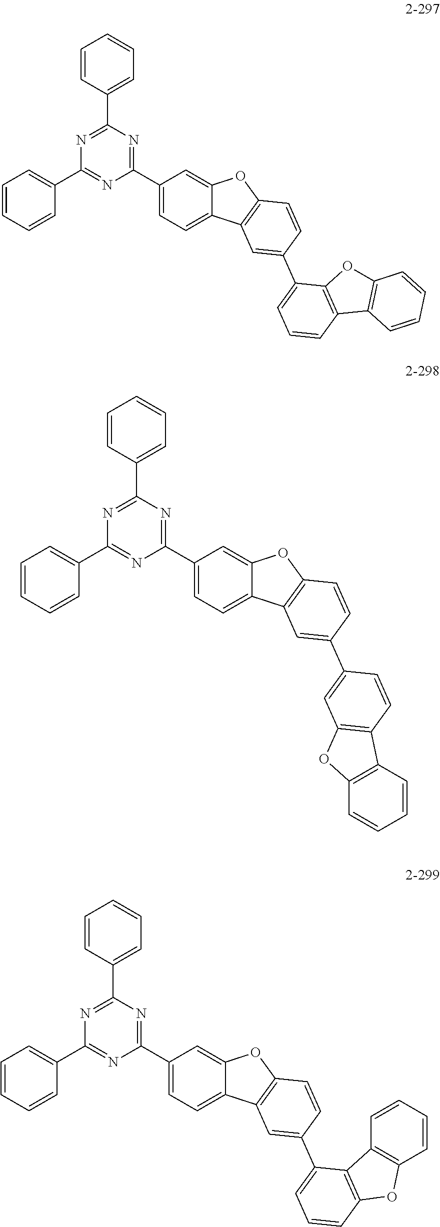 [SMILES: c1ccc(-c2nc(-c3ccccc3)nc(-c3ccc4c(c3)oc3ccc(-c5cccc6c5oc5ccccc56)cc34)n2)cc1 ; c1ccc(-c2nc(-c3ccccc3)nc(-c3ccc4c(c3)oc3ccc(-c5ccc6c(c5)oc5ccccc56)cc34)n2)cc1 ; c1ccc(-c2nc(-c3ccccc3)nc(-c3ccc4c(c3)oc3ccc(-c5cccc6oc7ccccc7c56)cc34)n2)cc1]

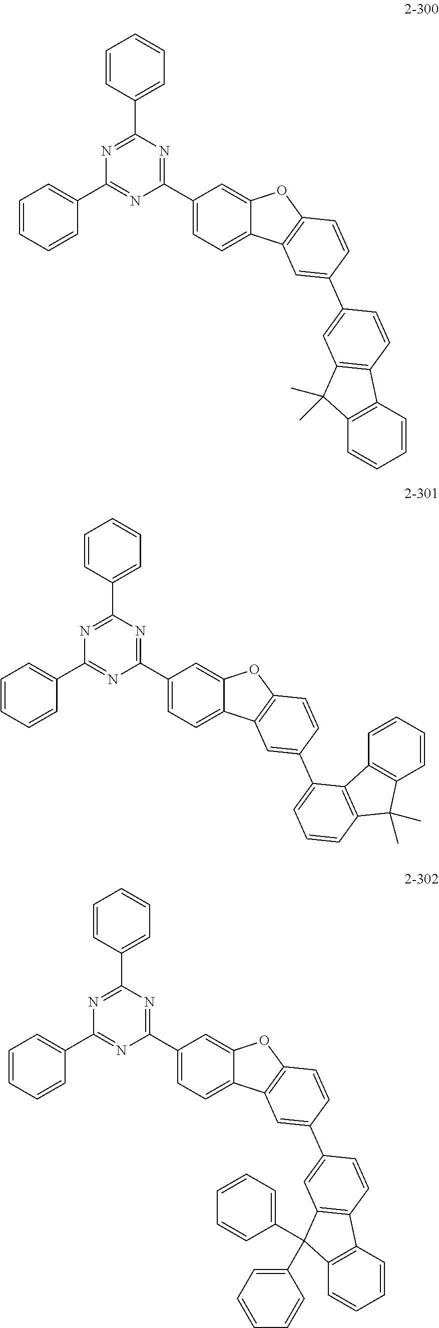 [SMILES: CC1(C)c2ccccc2-c2ccc(-c3ccc4oc5cc(-c6nc(-c7ccccc7)nc(-c7ccccc7)n6)ccc5c4c3)cc21 ; CC1(C)c2ccccc2-c2c(-c3ccc4oc5cc(-c6nc(-c7ccccc7)nc(-c7ccccc7)n6)ccc5c4c3)cccc21 ; c1ccc(-c2nc(-c3ccccc3)nc(-c3ccc4c(c3)oc3ccc(-c5ccc6c(c5)C(c5ccccc5)(c5ccccc5)c5ccccc5-6)cc34)n2)cc1]

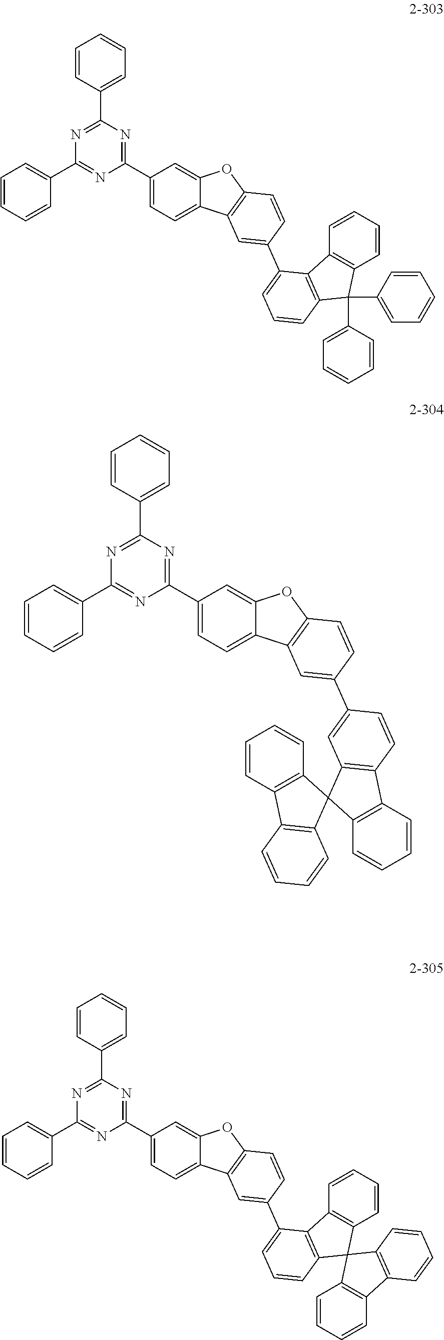 [SMILES: c1ccc(-c2nc(-c3ccccc3)nc(-c3ccc4c(c3)oc3ccc(-c5cccc6c5-c5ccccc5C6(c5ccccc5)c5ccccc5)cc34)n2)cc1 ; c1ccc(-c2nc(-c3ccccc3)nc(-c3ccc4c(c3)oc3ccc(-c5ccc6c(c5)C5(c7ccccc7-c7ccccc75)c5ccccc5-6)cc34)n2)cc1 ; c1ccc(-c2nc(-c3ccccc3)nc(-c3ccc4c(c3)oc3ccc(-c5cccc6c5-c5ccccc5C65c6ccccc6-c6ccccc65)cc34)n2)cc1]

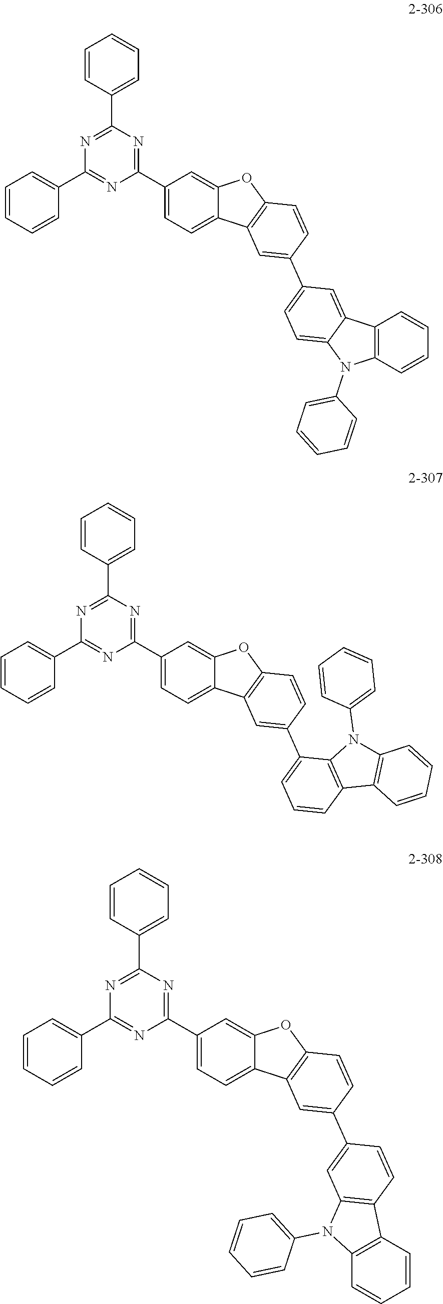 [SMILES: c1ccc(-c2nc(-c3ccccc3)nc(-c3ccc4c(c3)oc3ccc(-c5ccc6c(c5)c5ccccc5n6-c5ccccc5)cc34)n2)cc1 ; c1ccc(-c2nc(-c3ccccc3)nc(-c3ccc4c(c3)oc3ccc(-c5cccc6c7ccccc7n(-c7ccccc7)c56)cc34)n2)cc1 ; c1ccc(-c2nc(-c3ccccc3)nc(-c3ccc4c(c3)oc3ccc(-c5ccc6c7ccccc7n(-c7ccccc7)c6c5)cc34)n2)cc1]

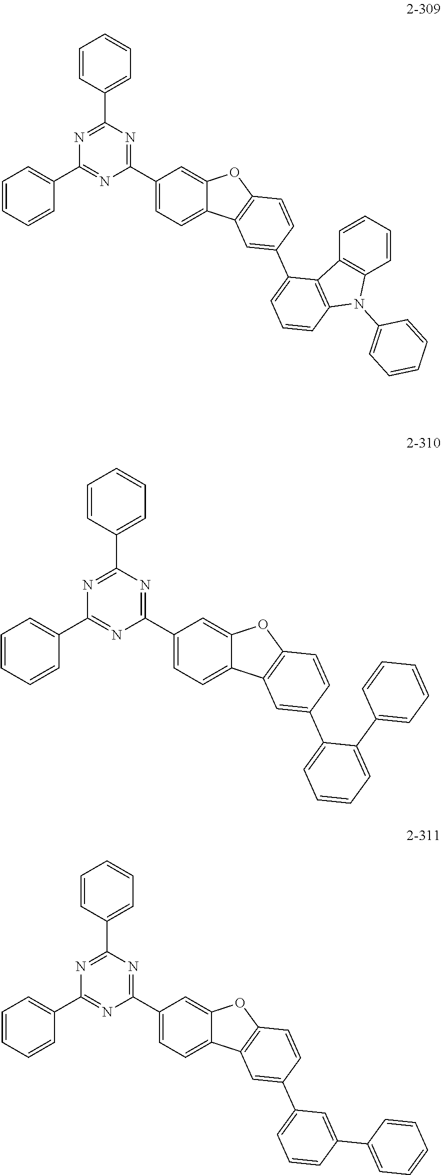 [SMILES: c1ccc(-c2nc(-c3ccccc3)nc(-c3ccc4c(c3)oc3ccc(-c5cccc6c5c5ccccc5n6-c5ccccc5)cc34)n2)cc1 ; c1ccc(-c2nc(-c3ccccc3)nc(-c3ccc4c(c3)oc3ccc(-c5ccccc5-c5ccccc5)cc34)n2)cc1 ; c1ccc(-c2cccc(-c3ccc4oc5cc(-c6nc(-c7ccccc7)nc(-c7ccccc7)n6)ccc5c4c3)c2)cc1]

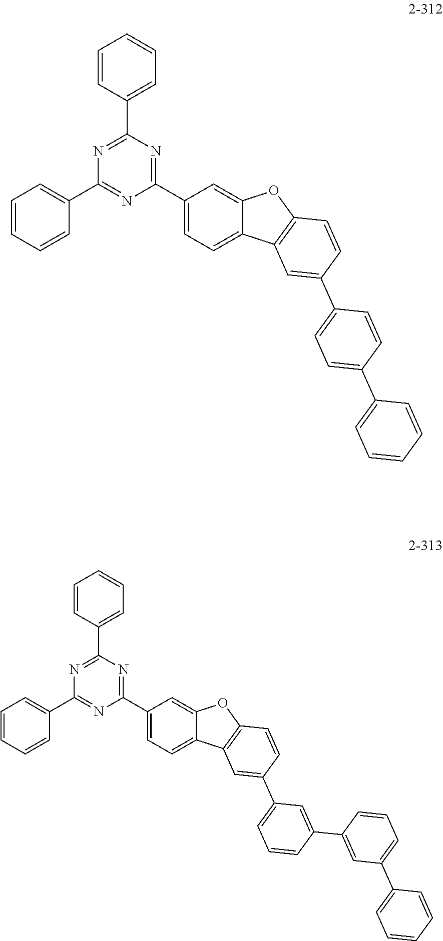 [SMILES: c1ccc(-c2ccc(-c3ccc4oc5cc(-c6nc(-c7ccccc7)nc(-c7ccccc7)n6)ccc5c4c3)cc2)cc1 ; c1ccc(-c2cccc(-c3cccc(-c4ccc5oc6cc(-c7nc(-c8ccccc8)nc(-c8ccccc8)n7)ccc6c5c4)c3)c2)cc1]

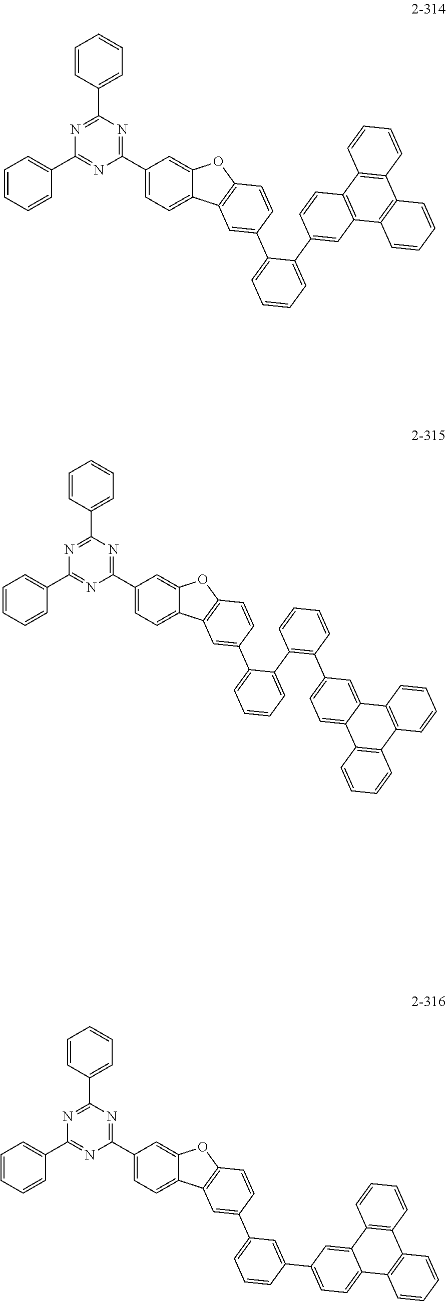 [SMILES: c1ccc(-c2nc(-c3ccccc3)nc(-c3ccc4c(c3)oc3ccc(-c5ccccc5-c5ccc6c7ccccc7c7ccccc7c6c5)cc34)n2)cc1 ; c1ccc(-c2nc(-c3ccccc3)nc(-c3ccc4c(c3)oc3ccc(-c5ccccc5-c5ccccc5-c5ccc6c7ccccc7c7ccccc7c6c5)cc34)n2)cc1 ; c1ccc(-c2nc(-c3ccccc3)nc(-c3ccc4c(c3)oc3ccc(-c5cccc(-c6ccc7c8ccccc8c8ccccc8c7c6)c5)cc34)n2)cc1]

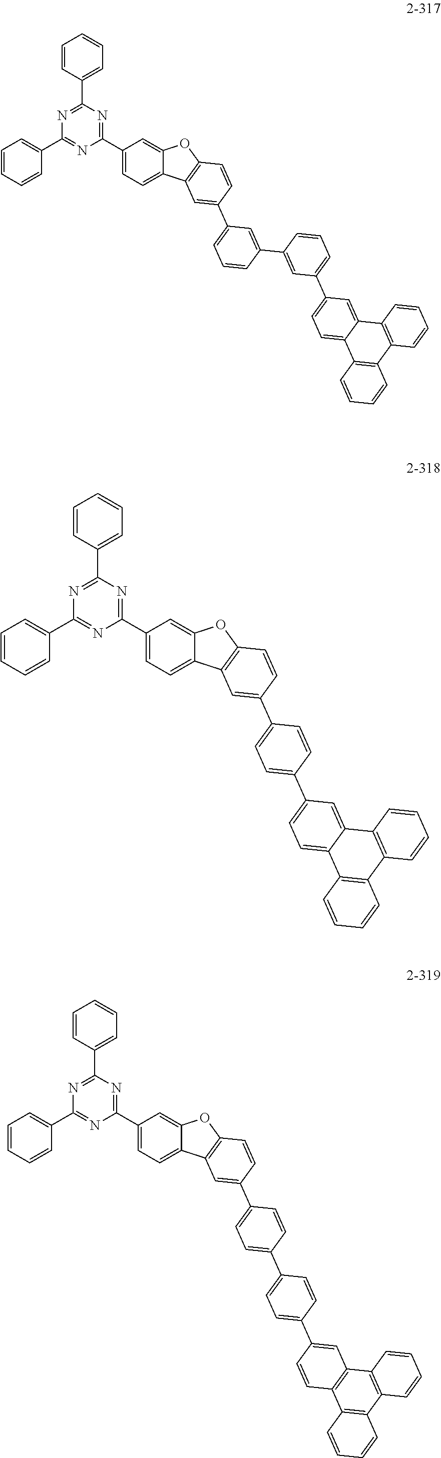 [SMILES: c1ccc(-c2nc(-c3ccccc3)nc(-c3ccc4c(c3)oc3ccc(-c5cccc(-c6cccc(-c7ccc8c9ccccc9c9ccccc9c8c7)c6)c5)cc34)n2)cc1 ; c1ccc(-c2nc(-c3ccccc3)nc(-c3ccc4c(c3)oc3ccc(-c5ccc(-c6ccc7c8ccccc8c8ccccc8c7c6)cc5)cc34)n2)cc1 ; c1ccc(-c2nc(-c3ccccc3)nc(-c3ccc4c(c3)oc3ccc(-c5ccc(-c6ccc(-c7ccc8c9ccccc9c9ccccc9c8c7)cc6)cc5)cc34)n2)cc1]

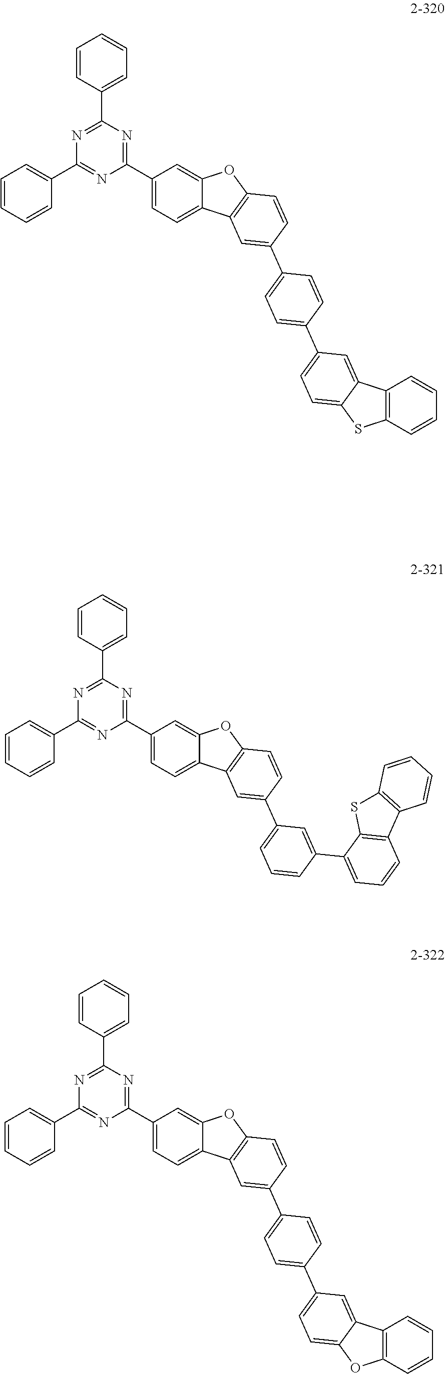 [SMILES: c1ccc(-c2nc(-c3ccccc3)nc(-c3ccc4c(c3)oc3ccc(-c5ccc(-c6ccc7sc8ccccc8c7c6)cc5)cc34)n2)cc1 ; c1ccc(-c2nc(-c3ccccc3)nc(-c3ccc4c(c3)oc3ccc(-c5cccc(-c6cccc7c6sc6ccccc67)c5)cc34)n2)cc1 ; c1ccc(-c2nc(-c3ccccc3)nc(-c3ccc4c(c3)oc3ccc(-c5ccc(-c6ccc7oc8ccccc8c7c6)cc5)cc34)n2)cc1]

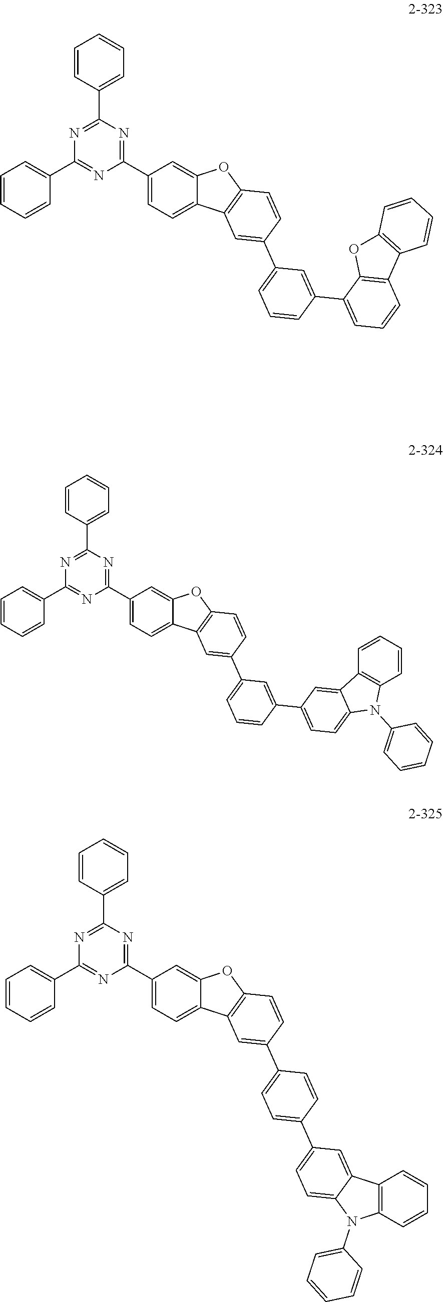 [SMILES: c1ccc(-c2nc(-c3ccccc3)nc(-c3ccc4c(c3)oc3ccc(-c5cccc(-c6cccc7c6oc6ccccc67)c5)cc34)n2)cc1 ; c1ccc(-c2nc(-c3ccccc3)nc(-c3ccc4c(c3)oc3ccc(-c5cccc(-c6ccc7c(c6)c6ccccc6n7-c6ccccc6)c5)cc34)n2)cc1 ; c1ccc(-c2nc(-c3ccccc3)nc(-c3ccc4c(c3)oc3ccc(-c5ccc(-c6ccc7c(c6)c6ccccc6n7-c6ccccc6)cc5)cc34)n2)cc1]

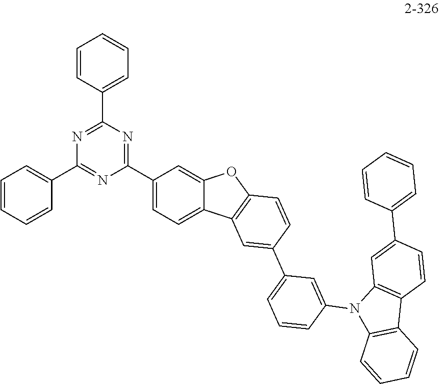 [SMILES: c1ccc(-c2ccc3c4ccccc4n(-c4cccc(-c5ccc6oc7cc(-c8nc(-c9ccccc9)nc(-c9ccccc9)n8)ccc7c6c5)c4)c3c2)cc1]

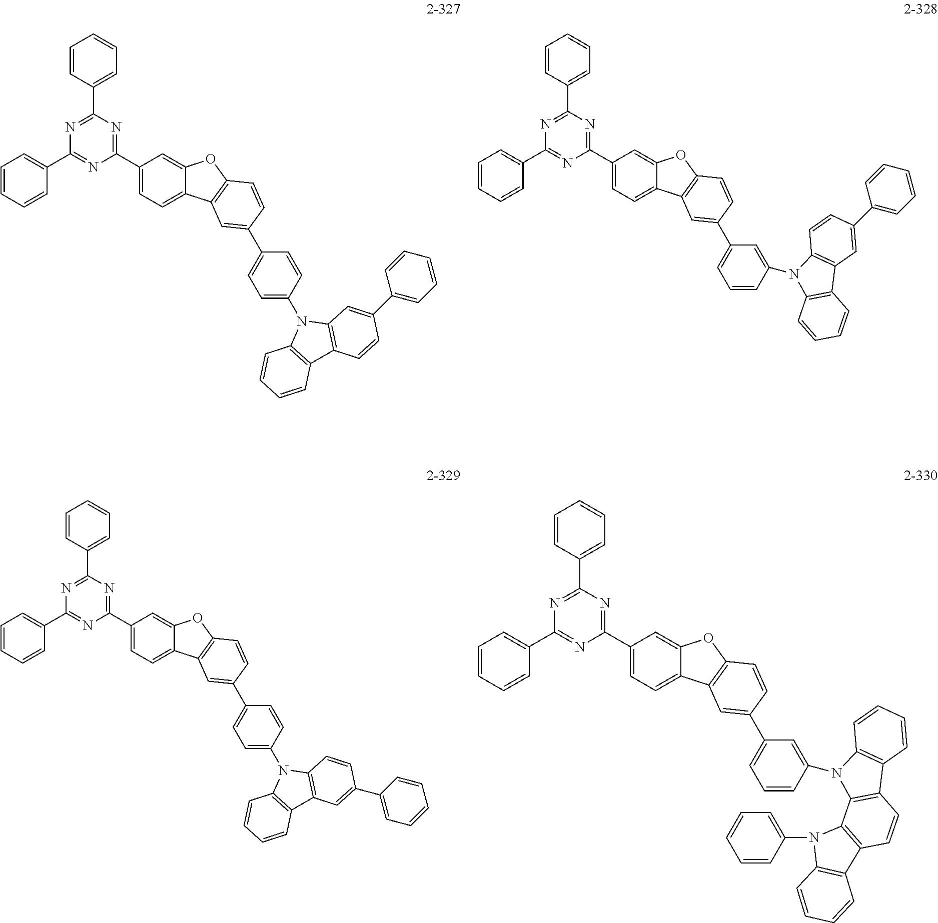 [SMILES: c1ccc(-c2ccc3c4ccccc4n(-c4ccc(-c5ccc6oc7cc(-c8nc(-c9ccccc9)nc(-c9ccccc9)n8)ccc7c6c5)cc4)c3c2)cc1 ; c1ccc(-c2ccc3c(c2)c2ccccc2n3-c2cccc(-c3ccc4oc5cc(-c6nc(-c7ccccc7)nc(-c7ccccc7)n6)ccc5c4c3)c2)cc1 ; c1ccc(-c2ccc3c(c2)c2ccccc2n3-c2ccc(-c3ccc4oc5cc(-c6nc(-c7ccccc7)nc(-c7ccccc7)n6)ccc5c4c3)cc2)cc1 ; c1ccc(-c2nc(-c3ccccc3)nc(-c3ccc4c(c3)oc3ccc(-c5cccc(-n6c7ccccc7c7ccc8c9ccccc9n(-c9ccccc9)c8c76)c5)cc34)n2)cc1]

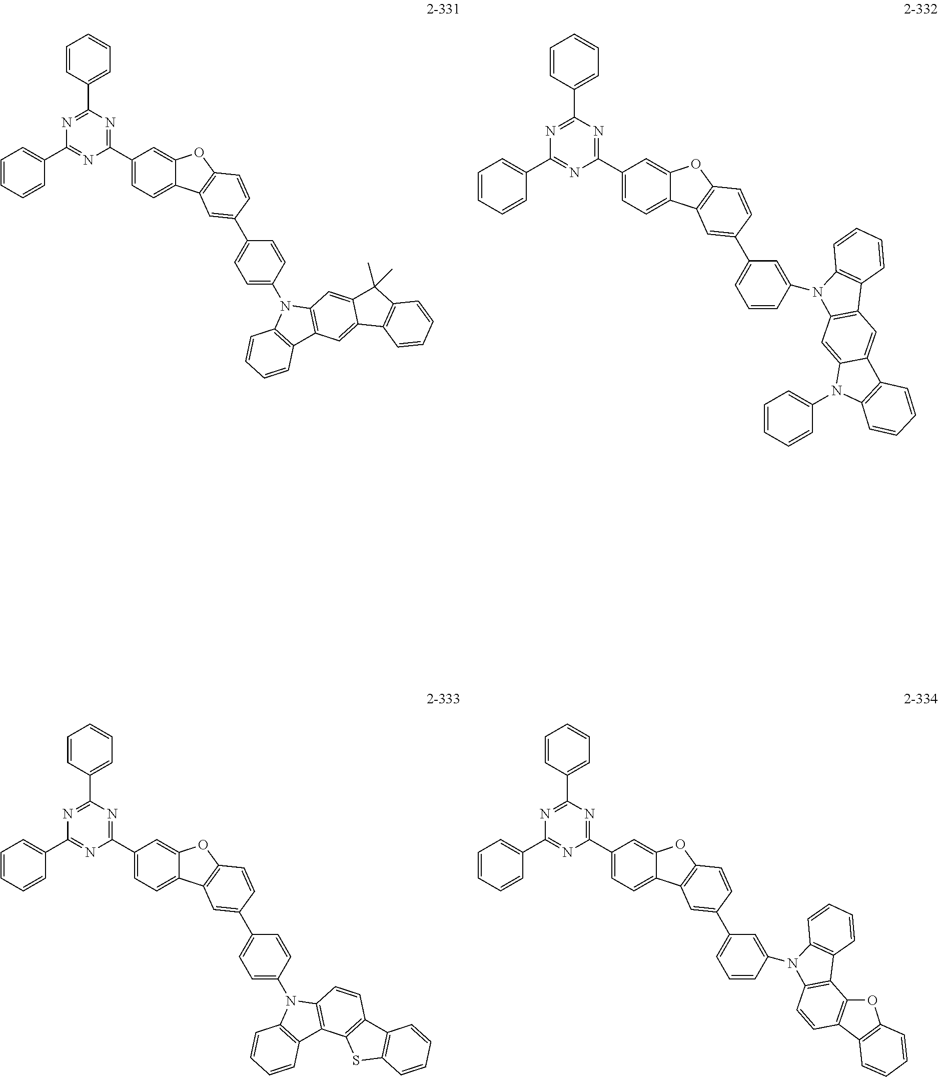 [SMILES: CC1(C)c2ccccc2-c2cc3c4ccccc4n(-c4ccc(-c5ccc6oc7cc(-c8nc(-c9ccccc9)nc(-c9ccccc9)n8)ccc7c6c5)cc4)c3cc21 ; c1ccc(-c2nc(-c3ccccc3)nc(-c3ccc4c(c3)oc3ccc(-c5cccc(-n6c7ccccc7c7cc8c9ccccc9n(-c9ccccc9)c8cc76)c5)cc34)n2)cc1 ; c1ccc(-c2nc(-c3ccccc3)nc(-c3ccc4c(c3)oc3ccc(-c5ccc(-n6c7ccccc7c7c8sc9ccccc9c8ccc76)cc5)cc34)n2)cc1 ; c1ccc(-c2nc(-c3ccccc3)nc(-c3ccc4c(c3)oc3ccc(-c5cccc(-n6c7ccccc7c7c8oc9ccccc9c8ccc76)c5)cc34)n2)cc1]

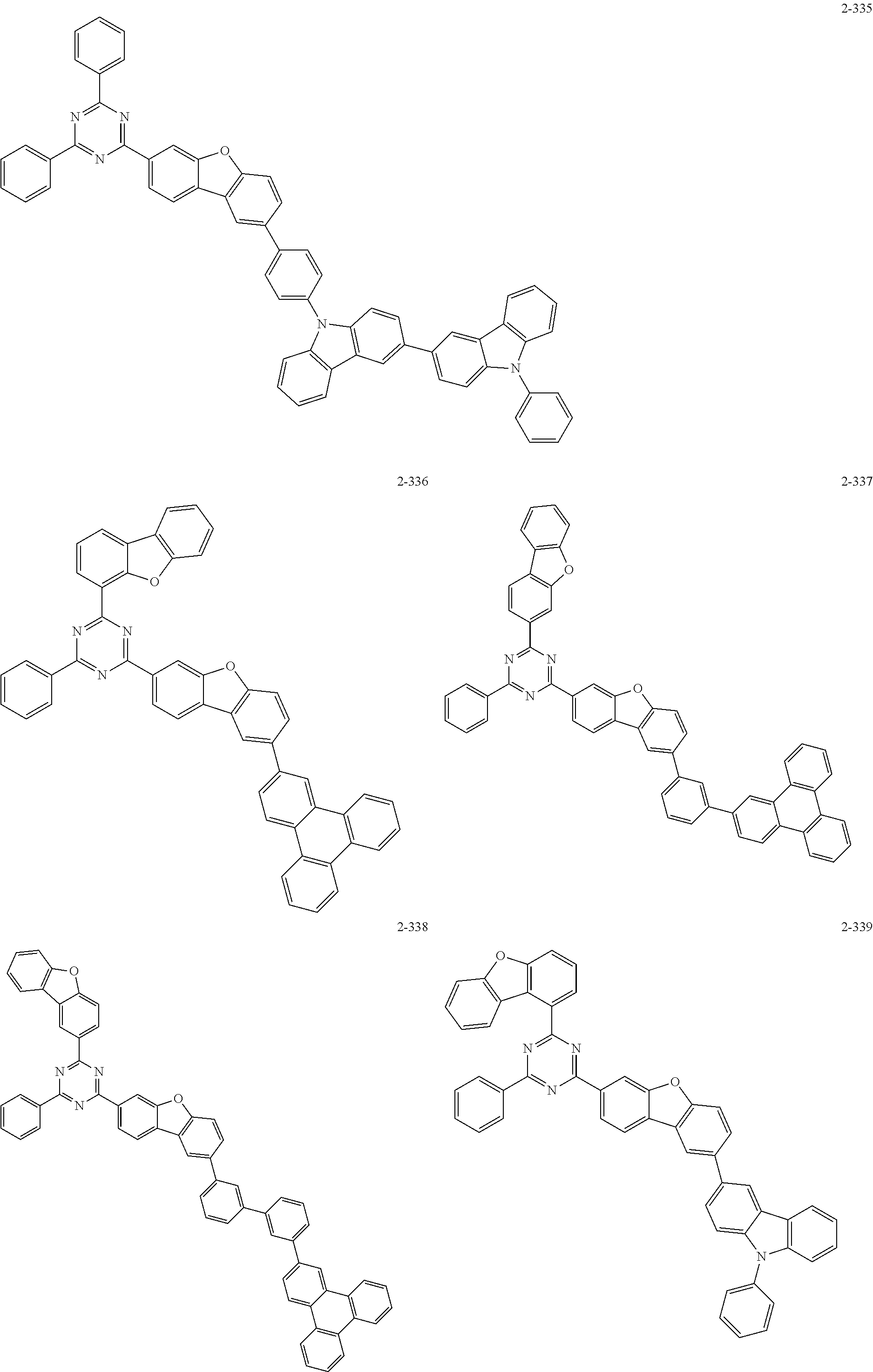

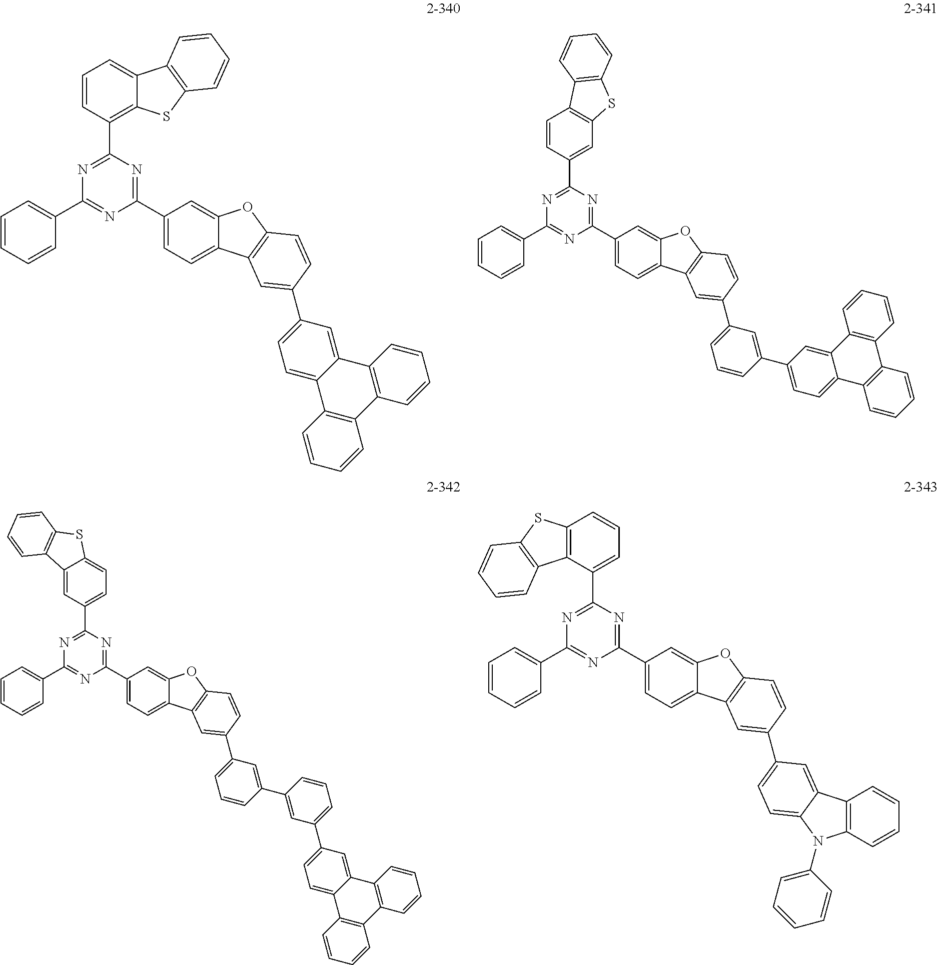 [SMILES: c1ccc(-c2nc(-c3ccc4c(c3)oc3ccc(-c5ccc6c7ccccc7c7ccccc7c6c5)cc34)nc(-c3cccc4c3sc3ccccc34)n2)cc1 ; c1ccc(-c2nc(-c3ccc4c(c3)oc3ccc(-c5cccc(-c6ccc7c8ccccc8c8ccccc8c7c6)c5)cc34)nc(-c3ccc4c(c3)sc3ccccc34)n2)cc1 ; c1ccc(-c2nc(-c3ccc4c(c3)oc3ccc(-c5cccc(-c6cccc(-c7ccc8c9ccccc9c9ccccc9c8c7)c6)c5)cc34)nc(-c3ccc4sc5ccccc5c4c3)n2)cc1 ; c1ccc(-c2nc(-c3ccc4c(c3)oc3ccc(-c5ccc6c(c5)c5ccccc5n6-c5ccccc5)cc34)nc(-c3cccc4sc5ccccc5c34)n2)cc1]

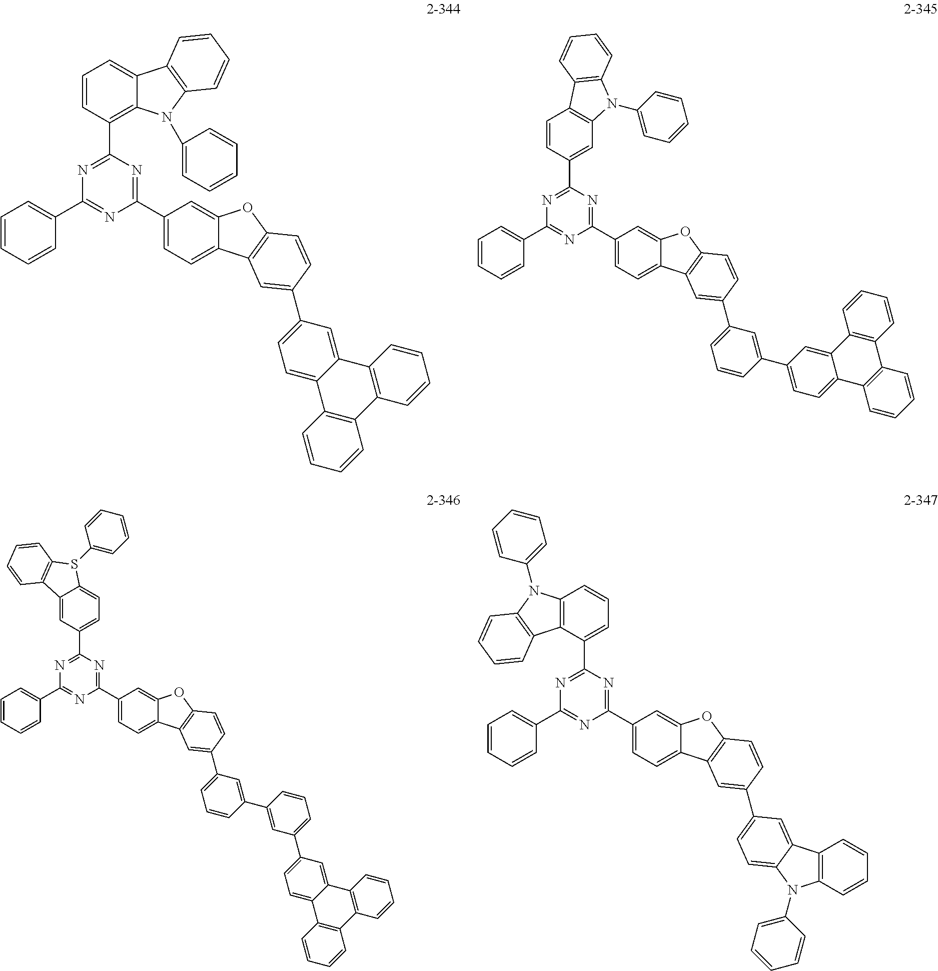

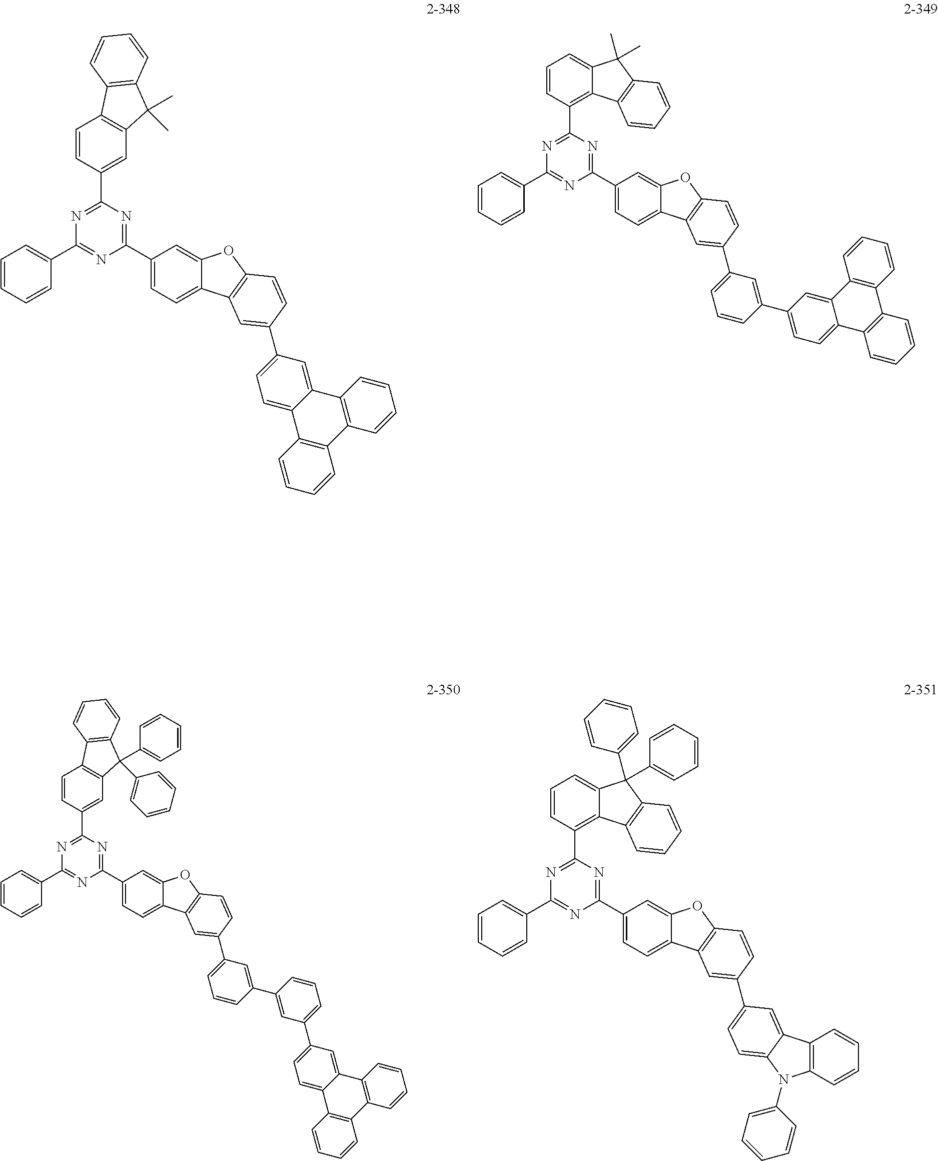 [SMILES: CC1(C)c2ccccc2-c2ccc(-c3nc(-c4ccccc4)nc(-c4ccc5c(c4)oc4ccc(-c6ccc7c8ccccc8c8ccccc8c7c6)cc45)n3)cc21 ; CC1(C)c2ccccc2-c2c(-c3nc(-c4ccccc4)nc(-c4ccc5c(c4)oc4ccc(-c6cccc(-c7ccc8c9ccccc9c9ccccc9c8c7)c6)cc45)n3)cccc21 ; c1ccc(-c2nc(-c3ccc4c(c3)C(c3ccccc3)(c3ccccc3)c3ccccc3-4)nc(-c3ccc4c(c3)oc3ccc(-c5cccc(-c6cccc(-c7ccc8c9ccccc9c9ccccc9c8c7)c6)c5)cc34)n2)cc1 ; c1ccc(-c2nc(-c3ccc4c(c3)oc3ccc(-c5ccc6c(c5)c5ccccc5n6-c5ccccc5)cc34)nc(-c3cccc4c3-c3ccccc3C4(c3ccccc3)c3ccccc3)n2)cc1]

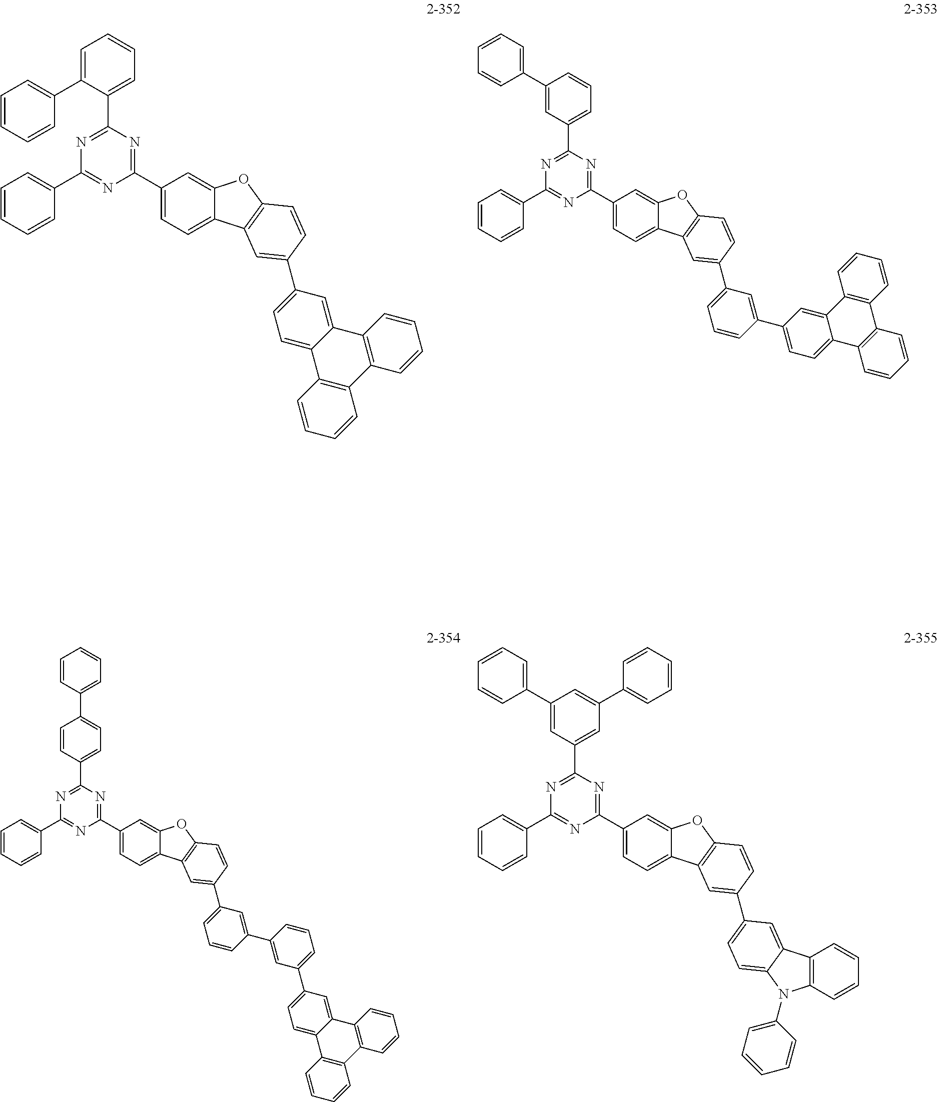 [SMILES: c1ccc(-c2nc(-c3ccc4c(c3)oc3ccc(-c5ccc6c7ccccc7c7ccccc7c6c5)cc34)nc(-c3ccccc3-c3ccccc3)n2)cc1 ; c1ccc(-c2cccc(-c3nc(-c4ccccc4)nc(-c4ccc5c(c4)oc4ccc(-c6cccc(-c7ccc8c9ccccc9c9ccccc9c8c7)c6)cc45)n3)c2)cc1 ; c1ccc(-c2ccc(-c3nc(-c4ccccc4)nc(-c4ccc5c(c4)oc4ccc(-c6cccc(-c7cccc(-c8ccc9c%10ccccc%10c%10ccccc%10c9c8)c7)c6)cc45)n3)cc2)cc1 ; c1ccc(-c2cc(-c3ccccc3)cc(-c3nc(-c4ccccc4)nc(-c4ccc5c(c4)oc4ccc(-c6ccc7c(c6)c6ccccc6n7-c6ccccc6)cc45)n3)c2)cc1]

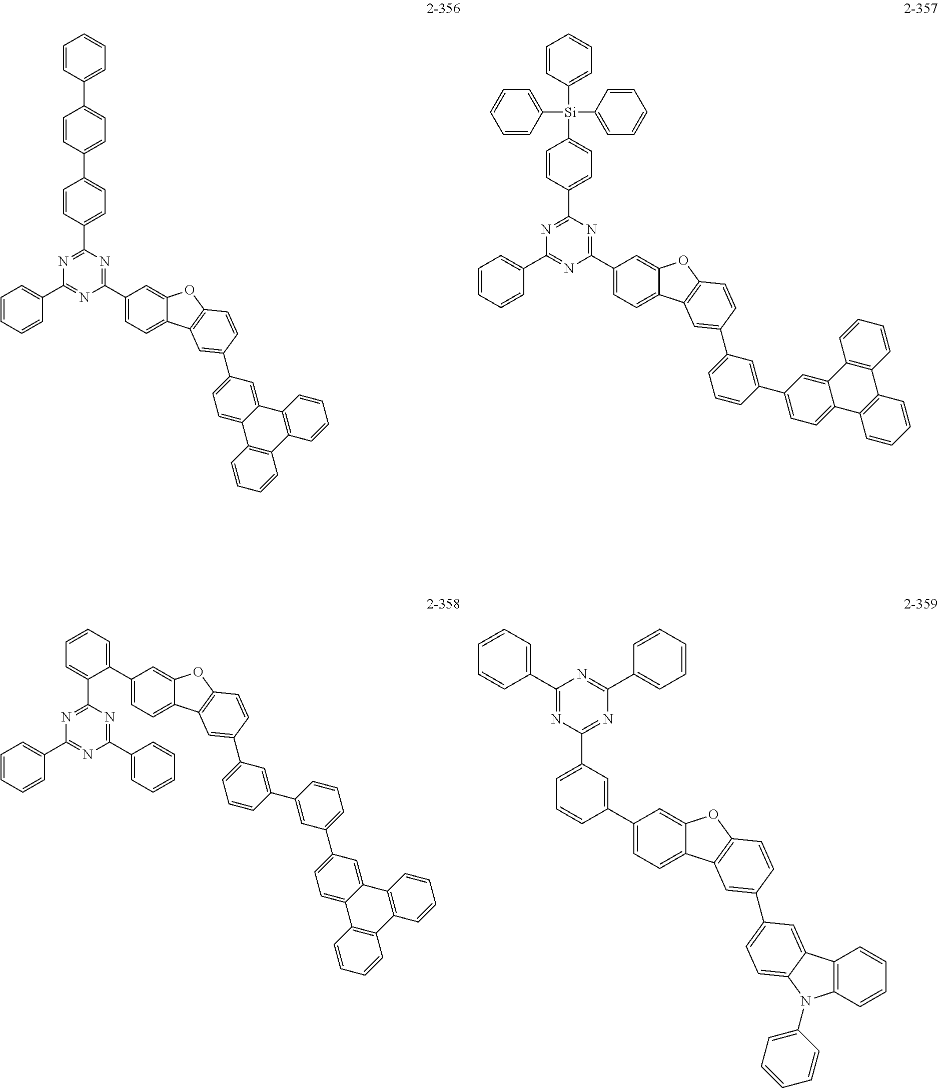 [SMILES: c1ccc(-c2ccc(-c3ccc(-c4nc(-c5ccccc5)nc(-c5ccc6c(c5)oc5ccc(-c7ccc8c9ccccc9c9ccccc9c8c7)cc56)n4)cc3)cc2)cc1 ; c1ccc(-c2nc(-c3ccc([Si](c4ccccc4)(c4ccccc4)c4ccccc4)cc3)nc(-c3ccc4c(c3)oc3ccc(-c5cccc(-c6ccc7c8ccccc8c8ccccc8c7c6)c5)cc34)n2)cc1 ; c1ccc(-c2nc(-c3ccccc3)nc(-c3ccccc3-c3ccc4c(c3)oc3ccc(-c5cccc(-c6cccc(-c7ccc8c9ccccc9c9ccccc9c8c7)c6)c5)cc34)n2)cc1 ; c1ccc(-c2nc(-c3ccccc3)nc(-c3cccc(-c4ccc5c(c4)oc4ccc(-c6ccc7c(c6)c6ccccc6n7-c6ccccc6)cc45)c3)n2)cc1]

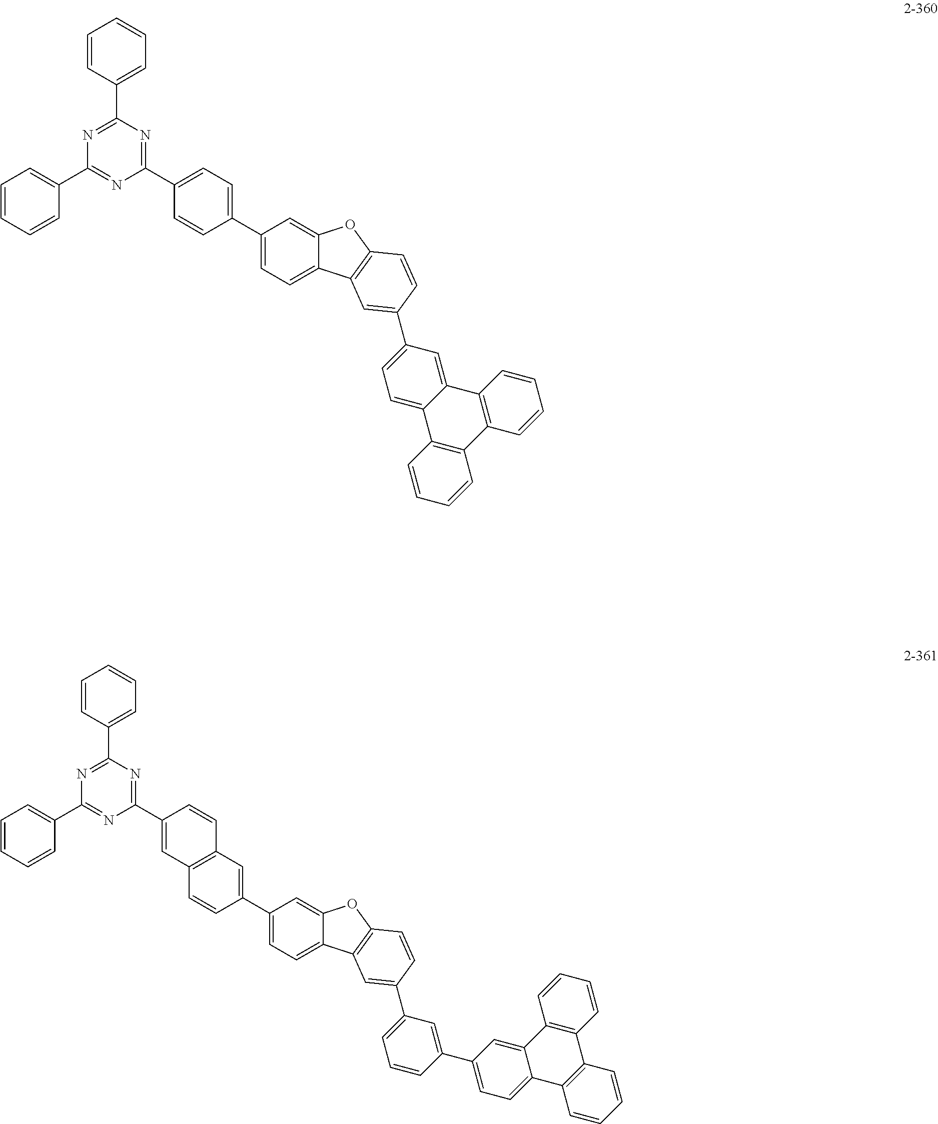 [SMILES: c1ccc(-c2nc(-c3ccccc3)nc(-c3ccc(-c4ccc5c(c4)oc4ccc(-c6ccc7c8ccccc8c8ccccc8c7c6)cc45)cc3)n2)cc1 ; c1ccc(-c2nc(-c3ccccc3)nc(-c3ccc4cc(-c5ccc6c(c5)oc5ccc(-c7cccc(-c8ccc9c%10ccccc%10c%10ccccc%10c9c8)c7)cc56)ccc4c3)n2)cc1]

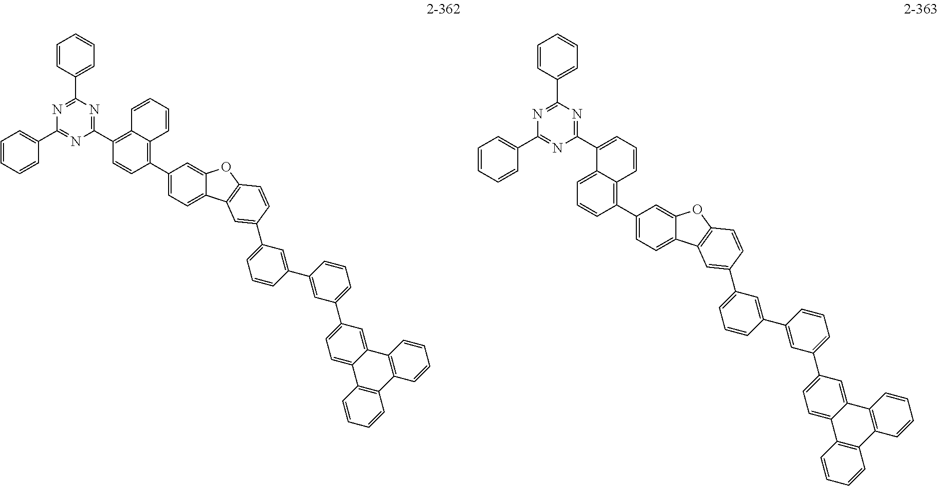 [SMILES: c1ccc(-c2nc(-c3ccccc3)nc(-c3ccc(-c4ccc5c(c4)oc4ccc(-c6cccc(-c7cccc(-c8ccc9c%10ccccc%10c%10ccccc%10c9c8)c7)c6)cc45)c4ccccc34)n2)cc1 ; c1ccc(-c2nc(-c3ccccc3)nc(-c3cccc4c(-c5ccc6c(c5)oc5ccc(-c7cccc(-c8cccc(-c9ccc%10c%11ccccc%11c%11ccccc%11c%10c9)c8)c7)cc56)cccc34)n2)cc1]

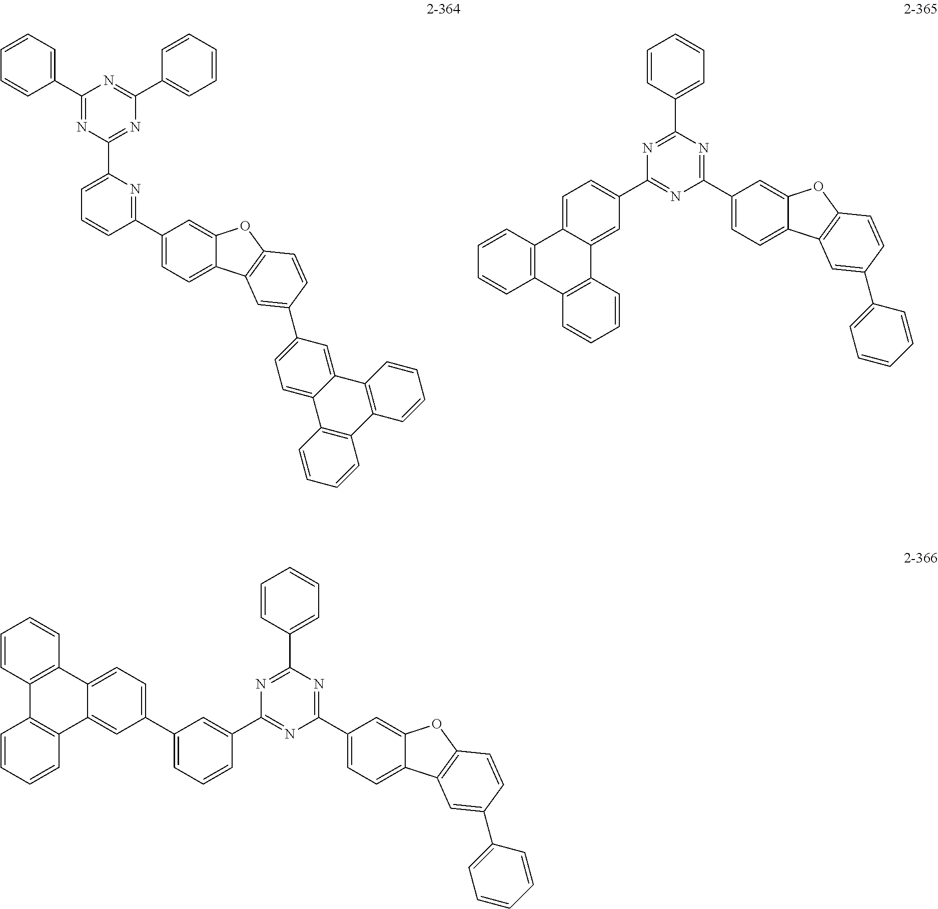 [SMILES: c1ccc(-c2nc(-c3ccccc3)nc(-c3cccc(-c4ccc5c(c4)oc4ccc(-c6ccc7c8ccccc8c8ccccc8c7c6)cc45)n3)n2)cc1 ; c1ccc(-c2ccc3oc4cc(-c5nc(-c6ccccc6)nc(-c6ccc7c8ccccc8c8ccccc8c7c6)n5)ccc4c3c2)cc1 ; c1ccc(-c2ccc3oc4cc(-c5nc(-c6ccccc6)nc(-c6cccc(-c7ccc8c9ccccc9c9ccccc9c8c7)c6)n5)ccc4c3c2)cc1]

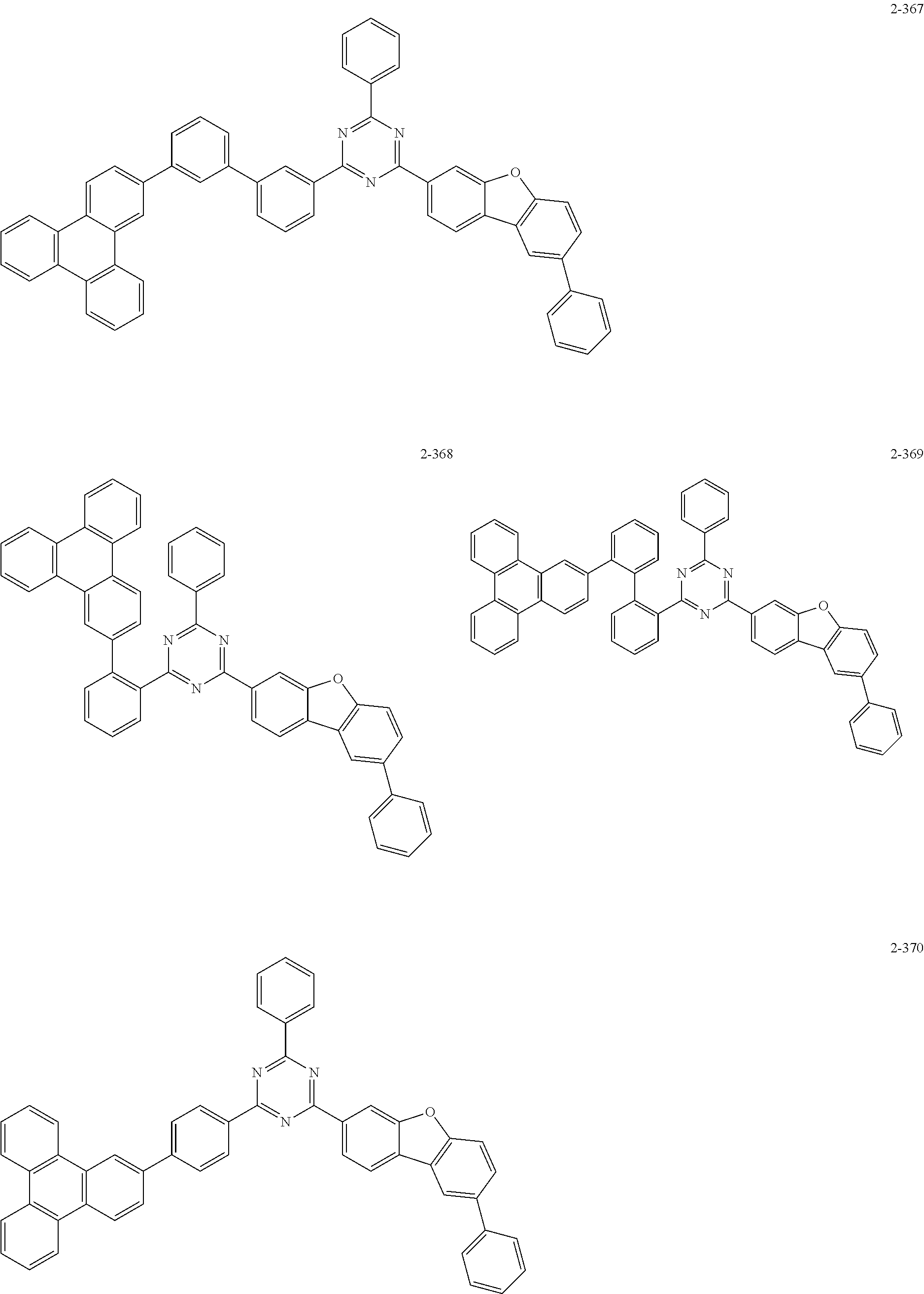 [SMILES: c1ccc(-c2ccc3oc4cc(-c5nc(-c6ccccc6)nc(-c6cccc(-c7cccc(-c8ccc9c%10ccccc%10c%10ccccc%10c9c8)c7)c6)n5)ccc4c3c2)cc1 ; c1ccc(-c2ccc3oc4cc(-c5nc(-c6ccccc6)nc(-c6ccccc6-c6ccc7c8ccccc8c8ccccc8c7c6)n5)ccc4c3c2)cc1 ; c1ccc(-c2ccc3oc4cc(-c5nc(-c6ccccc6)nc(-c6ccccc6-c6ccccc6-c6ccc7c8ccccc8c8ccccc8c7c6)n5)ccc4c3c2)cc1 ; c1ccc(-c2ccc3oc4cc(-c5nc(-c6ccccc6)nc(-c6ccc(-c7ccc8c9ccccc9c9ccccc9c8c7)cc6)n5)ccc4c3c2)cc1]

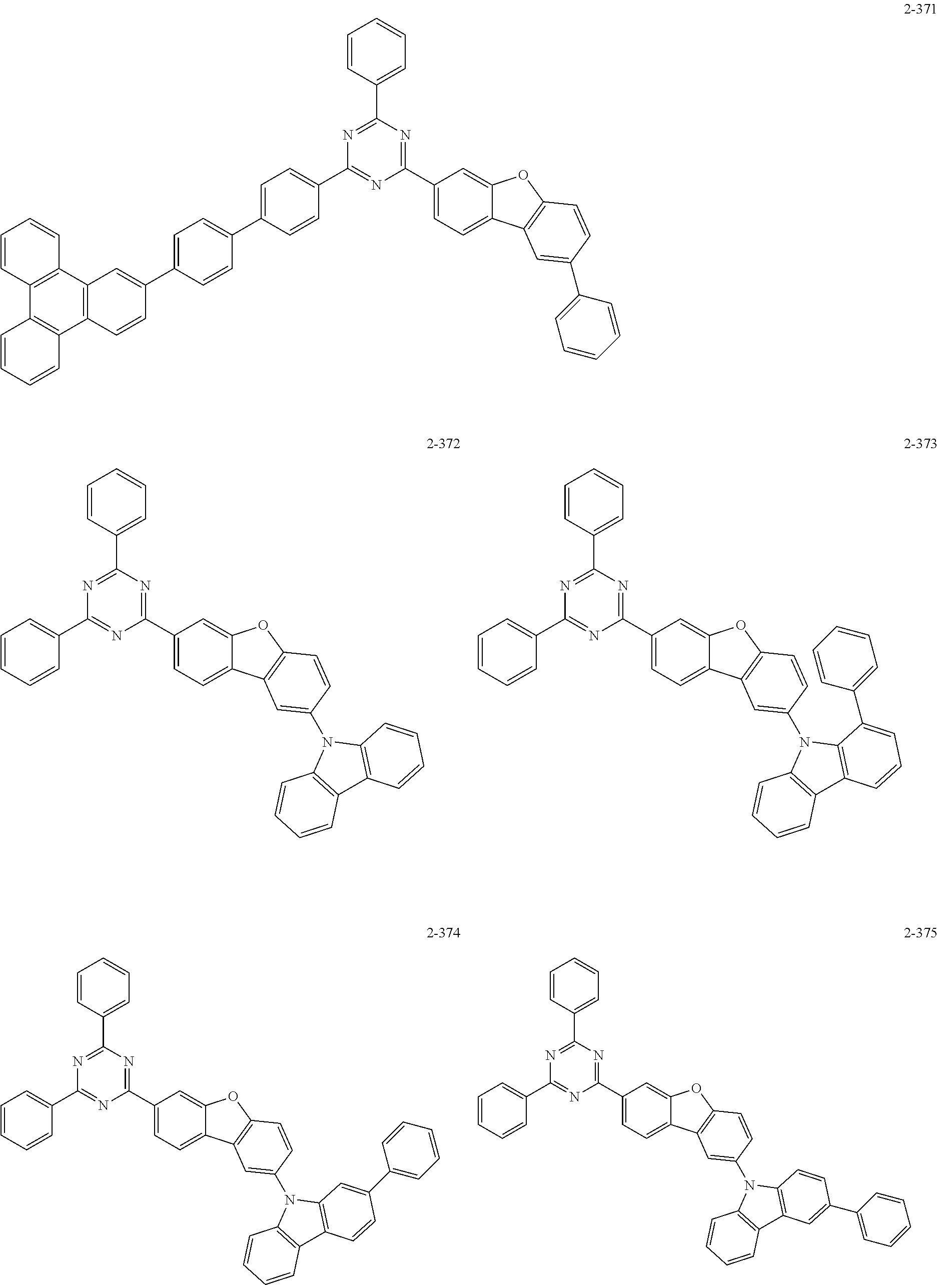 [SMILES: c1ccc(-c2ccc3oc4cc(-c5nc(-c6ccccc6)nc(-c6ccc(-c7ccc(-c8ccc9c%10ccccc%10c%10ccccc%10c9c8)cc7)cc6)n5)ccc4c3c2)cc1 ; c1ccc(-c2nc(-c3ccccc3)nc(-c3ccc4c(c3)oc3ccc(-n5c6ccccc6c6ccccc65)cc34)n2)cc1 ; c1ccc(-c2nc(-c3ccccc3)nc(-c3ccc4c(c3)oc3ccc(-n5c6ccccc6c6cccc(-c7ccccc7)c65)cc34)n2)cc1 ; c1ccc(-c2ccc3c4ccccc4n(-c4ccc5oc6cc(-c7nc(-c8ccccc8)nc(-c8ccccc8)n7)ccc6c5c4)c3c2)cc1 ; c1ccc(-c2ccc3c(c2)c2ccccc2n3-c2ccc3oc4cc(-c5nc(-c6ccccc6)nc(-c6ccccc6)n5)ccc4c3c2)cc1]

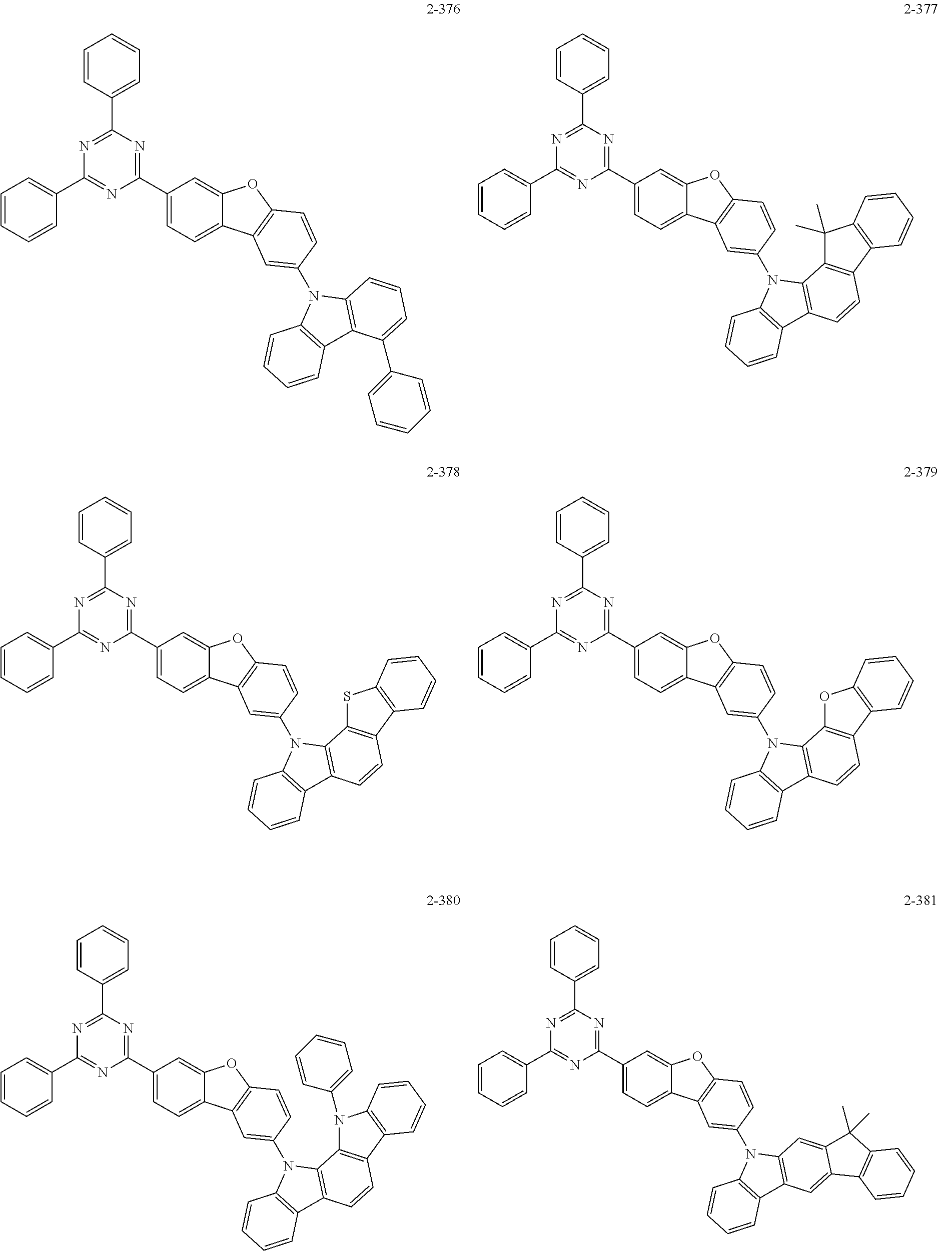

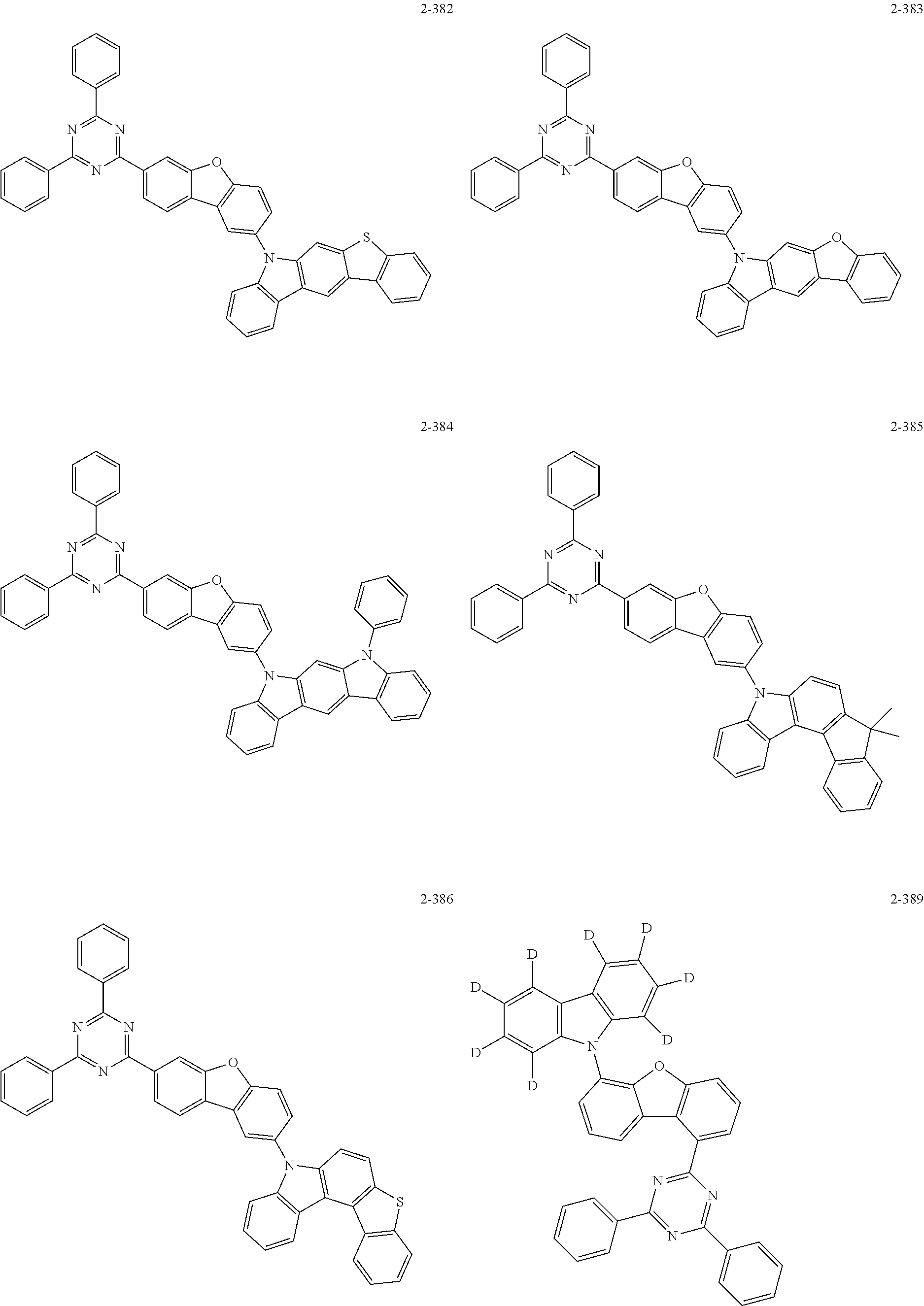

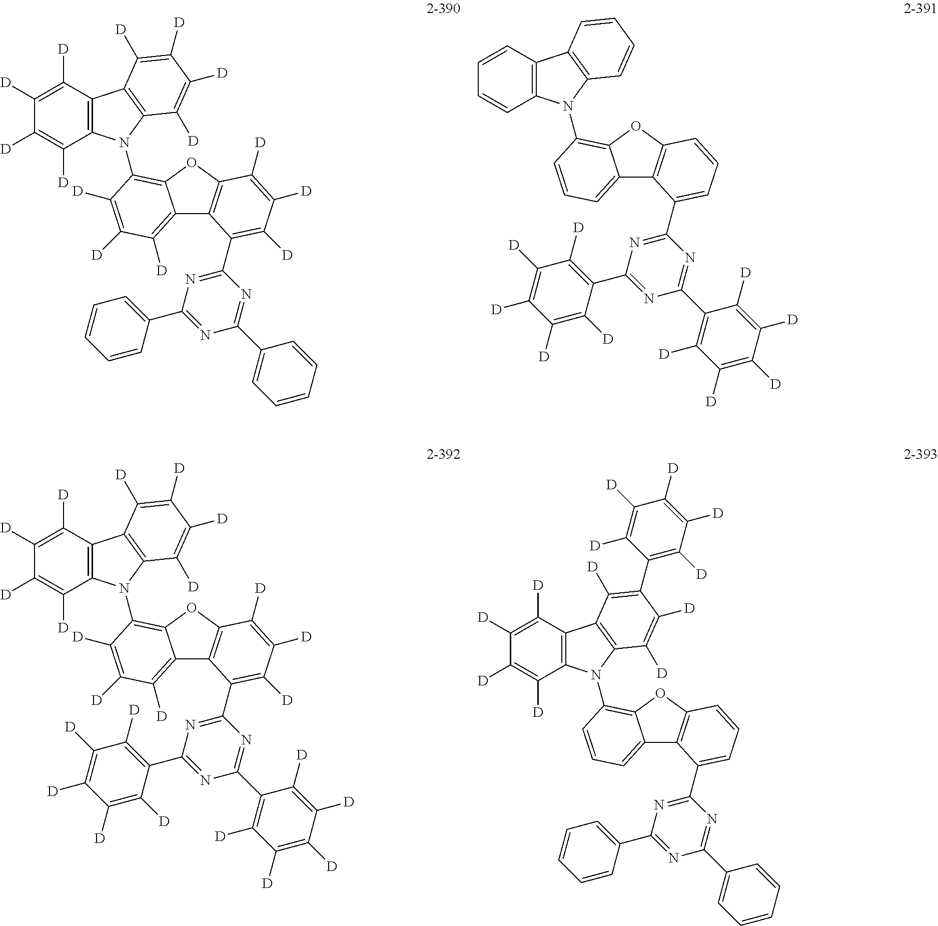

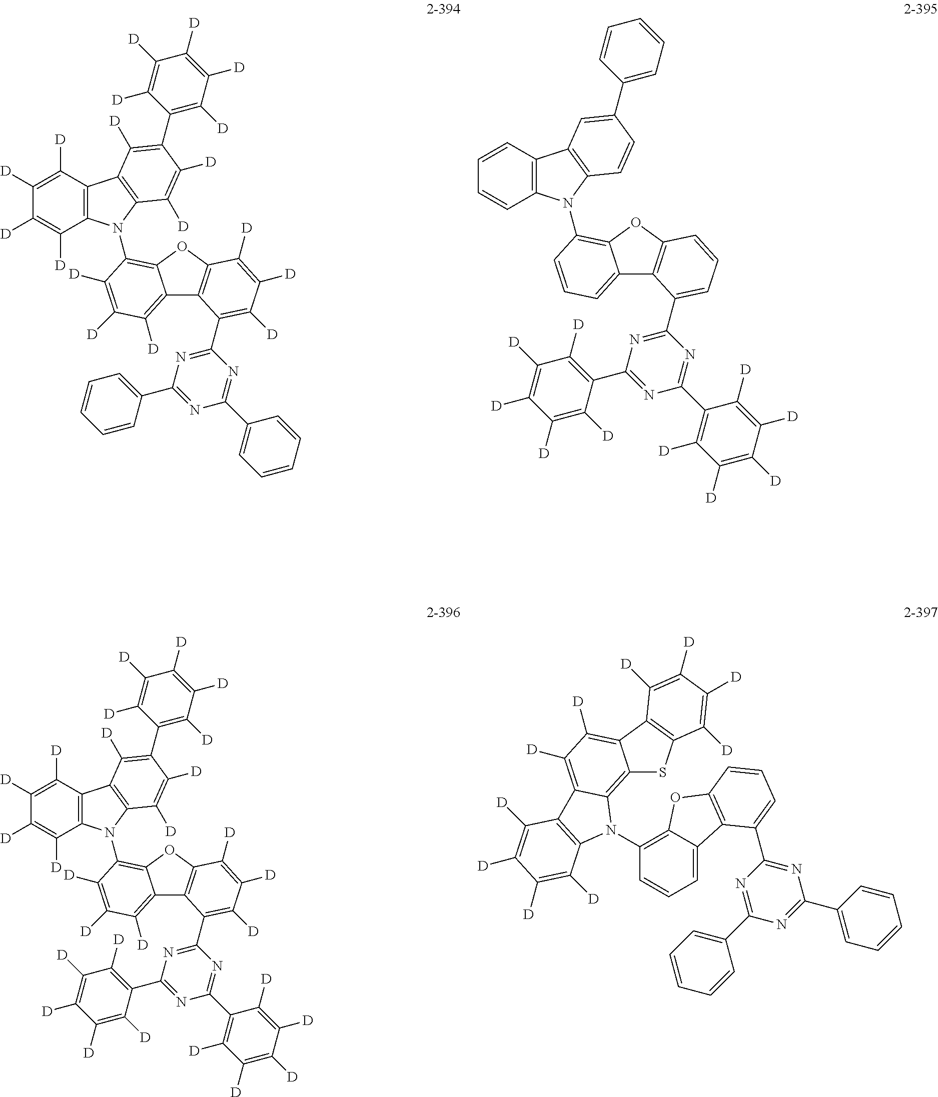

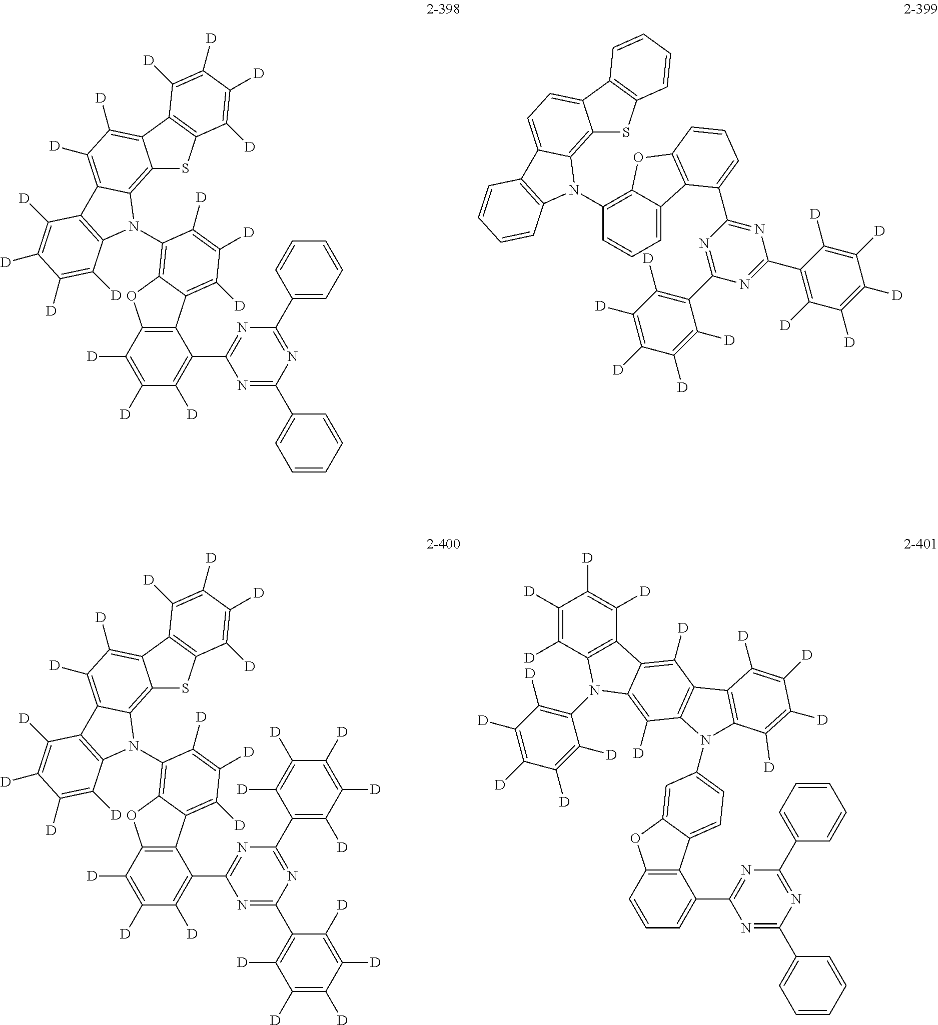

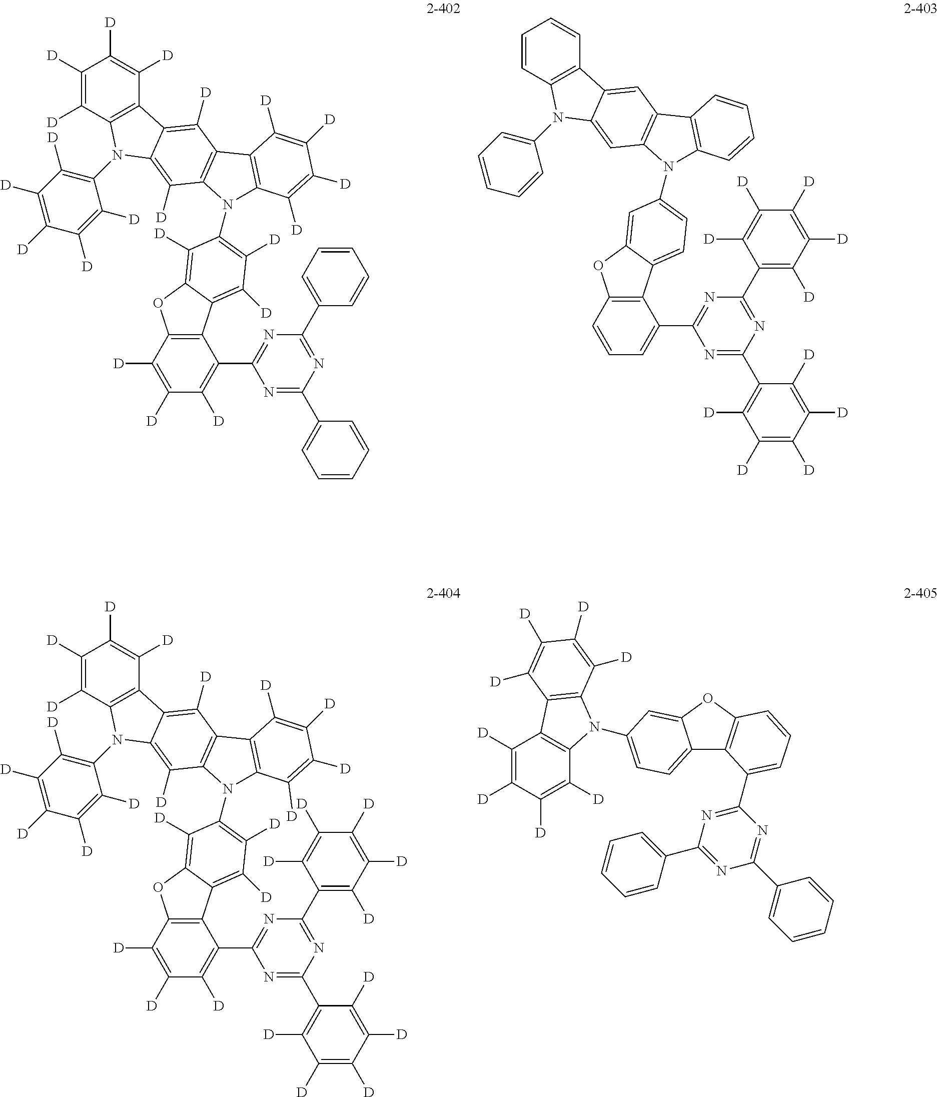

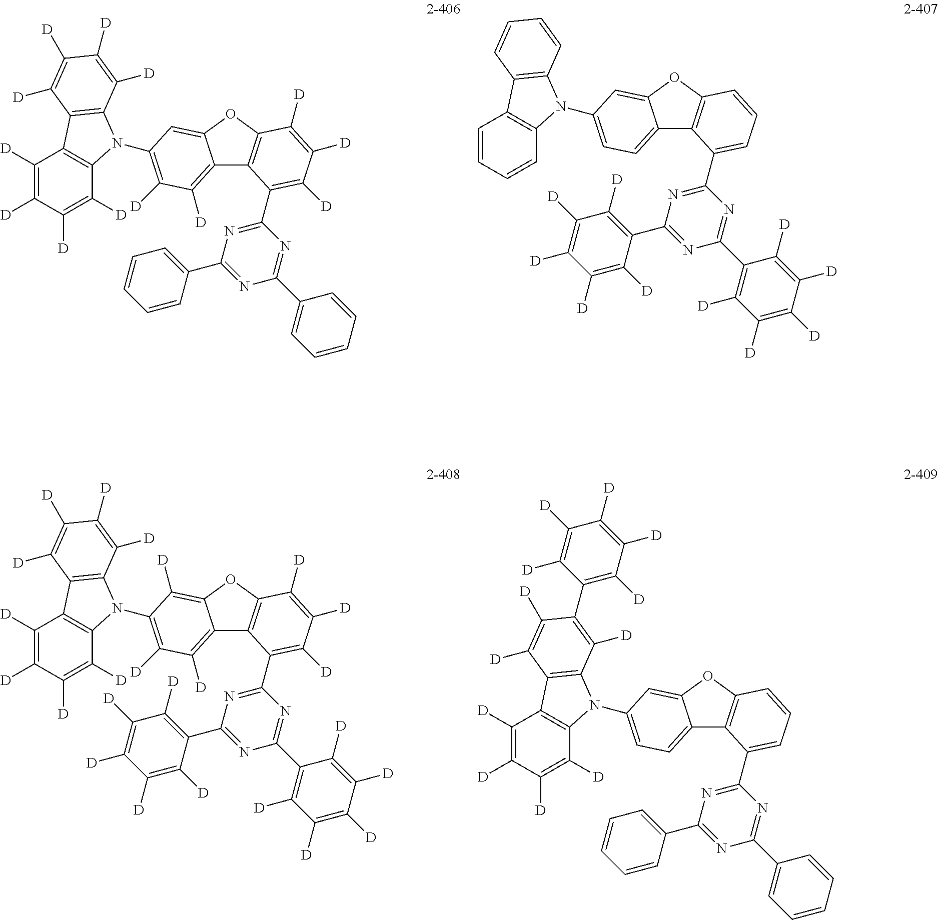

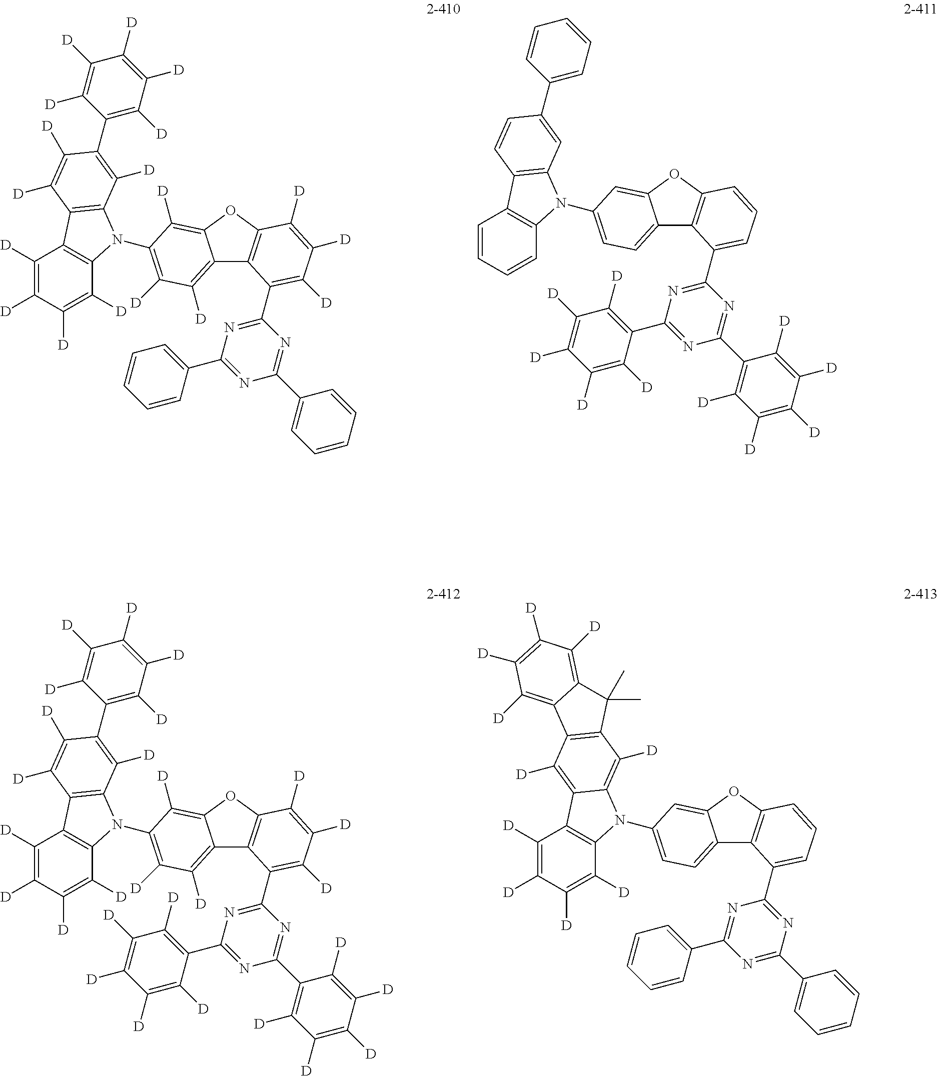

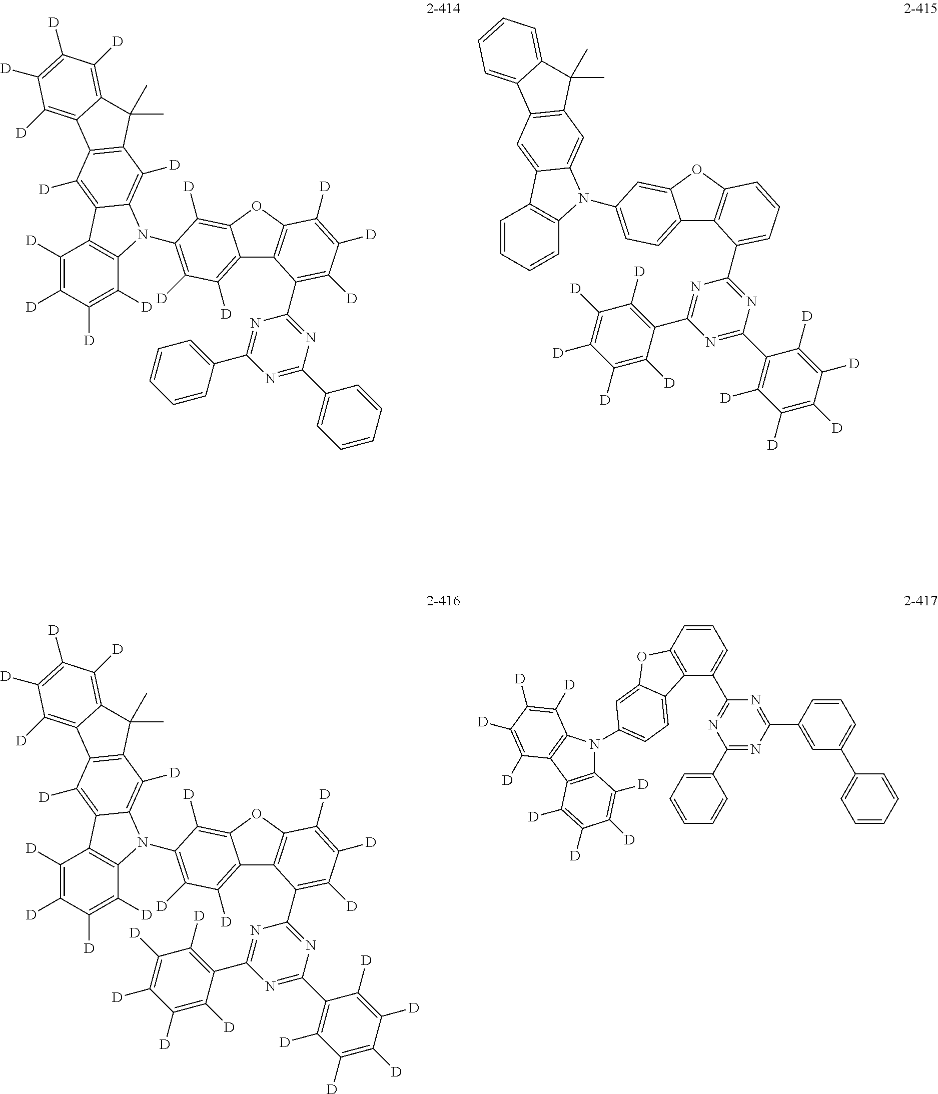

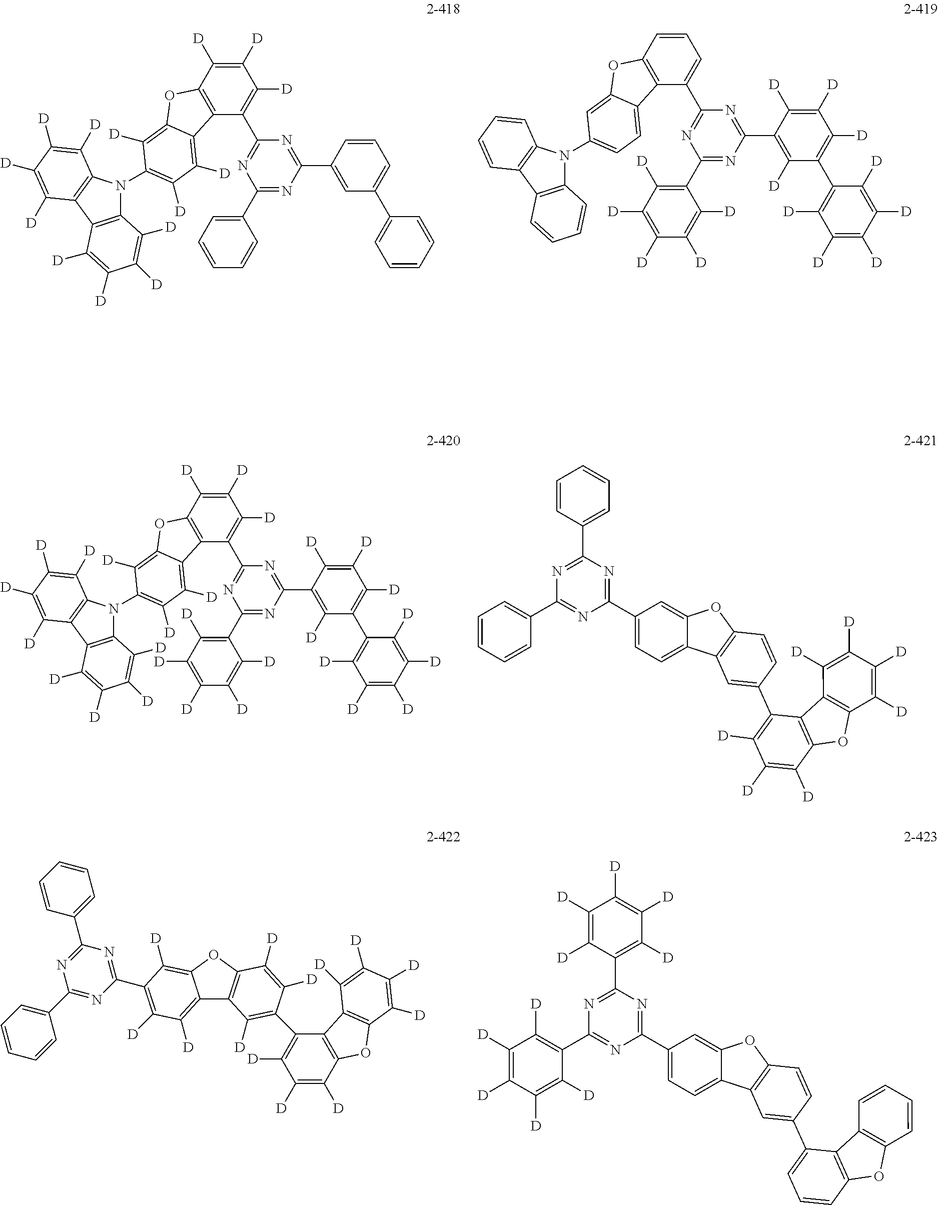

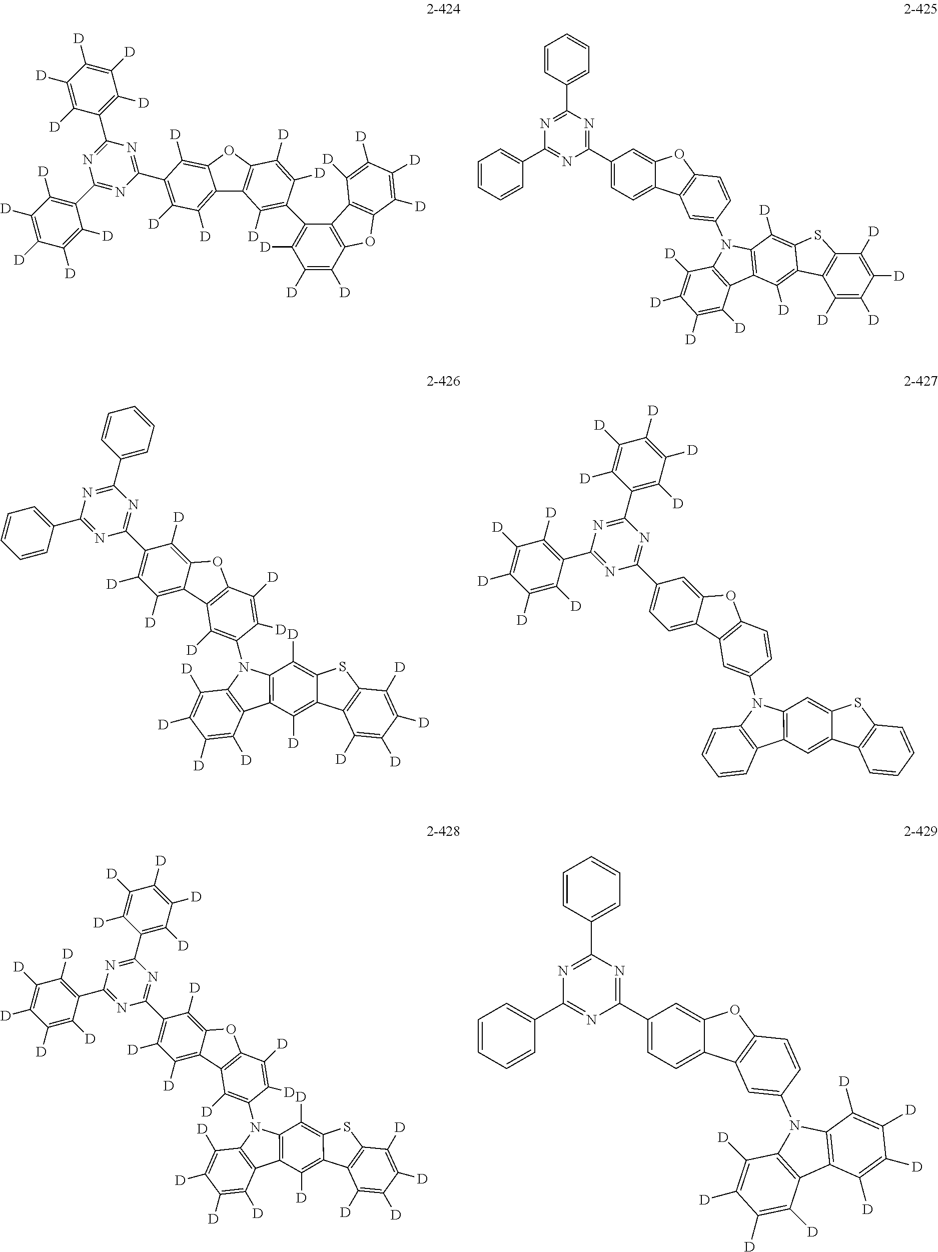

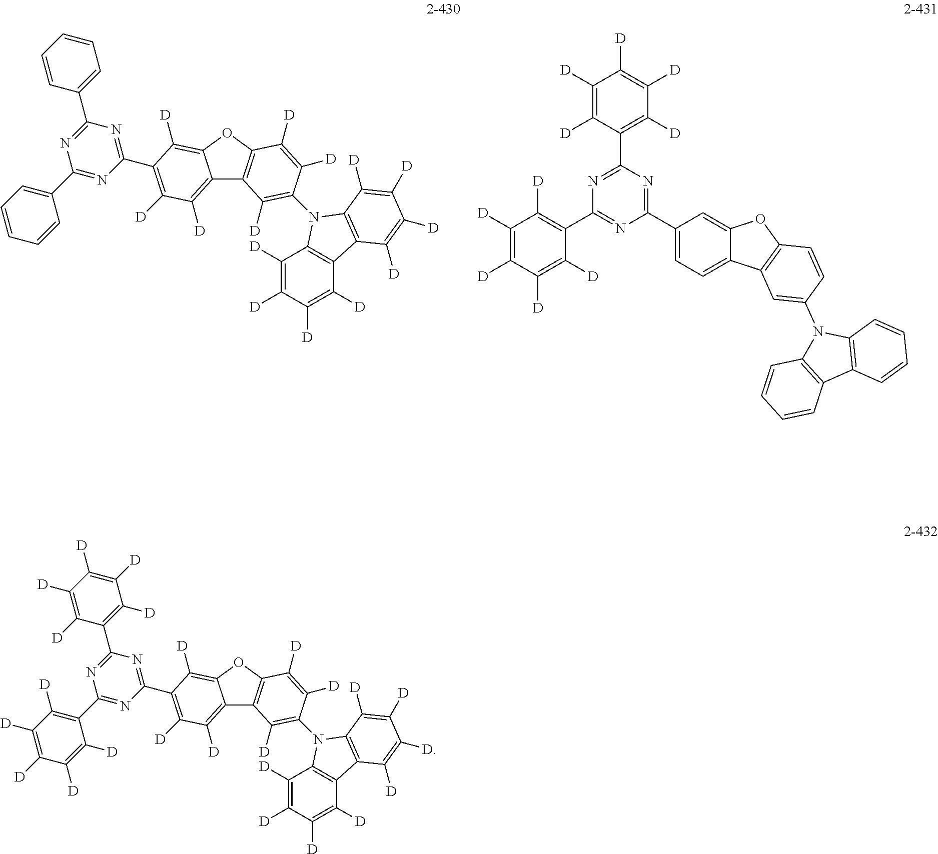

15. A composition for an organic material layer of an organic light emitting device, the composition comprising the heterocyclic compound represented by Chemical Formula 1 according to claim 1 and a heterocyclic compound represented by the following Chemical Formula 2: 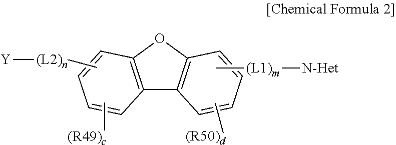
in Chemical Formula 2,
N-Het is a monocyclic or polycyclic heterocyclic group which is substituted or unsubstituted and comprises one or more N's,
L1 and L2 are the same as or different from each other, and are each independently a direct bond; a substituted or unsubstituted arylene group having 6 to 60 carbon atoms; or a substituted or unsubstituted heteroarylene group having 2 to 60 carbon atoms, m and n are each an integer from 0 to 3, when m is 2 or higher, L1's are the same as or different from each other, and when n is 2 or higher, L2's are the same as or different from each other,
R49 and R50 are the same as or different from each other, and are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or a substituted or unsubstituted hetero ring, c and d are each an integer from 0 to 3, when c is 2 or higher, R49's are the same as or different from each other, and when d is 2 or higher, R50's are the same as or different from each other, and
Y is a hole transporting group; or a substituted or unsubstituted aryl group having 6 to 60 carbon atoms.

16. The composition of claim 15, wherein a weight ratio of the heterocyclic compound represented by Chemical Formula 1: the heterocyclic compound represented by Chemical Formula 2 in the composition is 1:10 to 10:1.
